(19) United States
(12) Patent Application Publication
(10) Pub. No.: US 2026/0184761 A1
(43) Pub. Date: Jul. 2, 2026

(54) CALR AND JAK2 T-CELL RECEPTORS

(71) Applicant: Janssen Biotech, Inc., Horsham, PA (US)

(72) Inventors: Rebecca HANSON, Harleysville, PA (US); Vinod KRISHNA, Philadelphia, PA (US); Manuel Alejandro SEPULVEDA, West Windsor, NJ (US); Patrick WILKINSON, Collegeville, PA (US)

(21) Appl. No.: 19/566,278

(22) Filed: Mar. 13, 2026

Related U.S. Application Data
(63) Continuation of application No. 17/712,661, filed on Apr. 4, 2022.
(60) Provisional application No. 63/170,711, filed on Apr. 5, 2021.

(51) Int. Cl. C07K 14/725; C12N 15/86

(52) U.S. Cl. CPC C07K 14/7051; C12N 15/86

(57) ABSTRACT

Described herein are T-cell receptors (TCRs) that bind to CALR or JAK2 antigens. Also described are T-cell receptors (TCRs), polynucleotides, vectors that encode the TCRs, and cells comprising the TCRs, and methods of treatment.

### CLAIM OF PRIORITY

This application is a continuation of U.S. application Ser. No. 17/712,661, filed on Apr. 4, 2022, which claims priority to U.S. Provisional Application No. 63/170,711, filed on Apr. 5, 2021, the disclosures of each of which are hereby incorporated by reference in their entireties.

### SEQUENCE LISTING

The contents of the electronic sequence listing (103693.004414_SL.xml; Size: 1,035,730 bytes; and Date of Creation: Mar. 12, 2025) is herein incorporated by reference in its entirety.

### TECHNICAL FIELD

Provided are T-cell receptors (TCRs) that bind to CALR or JAK2 antigens. Also provided are TCRs, polynucleotides and vectors that encode the TCRs, cells comprising the TCRs, and methods of treatment.

### BACKGROUND

Janus Kinase 2 (JAK2) is a cytoplasmic protein involved in a signaling pathway known to regulate cell growth and proliferation, mainly through cytokine receptors on the cell surface. This pathway is important for generation and differentiation of blood cells from hemopoietic stem cells in the bone marrow. Lack of JAK2 is embryonic lethal due to lack of erythropoiesis (red blood cell production). JAK2 mutations have been corelated with myeloproliferative neoplasms (MPNs), such as polycythemia vera (PV), essential thromocythemia (ET), and primary myelofibrosis (PM). The most clinically relevant mutation is V617F, where the valine at amino acid position 617 is replaced by the amino acid phenylalanine. This mutation is an activating mutation making cells more sensitive to growth factors, leading to increased blood cell proliferation in these diseases. The prevalence of this mutation in MPN is high. Almost 100% of PV, 50% of PM, and 60% of ET patients harbor this mutation. The current standard of care treatment for MPN is Ruxolitinib, a JAK inhibitor, but this treatment is not curative. An unmet medical need still remains for treatment of these diseases.

### SUMMARY

Described herein are T-cell receptors (TCRs) comprising an alpha chain and a beta chain, wherein the alpha chain comprises a complementarity determining region 3 (CDR3) comprising an amino acid sequence provided in Table 4, and the beta chain comprises a CDR3 comprising a corresponding amino acid sequence provided in Table 5 (i.e. the beta chain CDR3 provided in Table 5 is in the same row number (row 1, for example) as the alpha chain CDR3 provided in Table 4 (row 1, for example)).

Also described herein are TCRs comprising an alpha chain and a beta chain, wherein the alpha chain comprises a complementarity determining region 1 (CDR1) comprising an amino acid sequence provided in Table 4, a complementarity determining region 2 (CDR2) comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5 (i.e. the beta chain CDRs provided in Table 5 are in the same row number (row 1, for example) as the alpha chain CDRs provided in Table 4 (row 1, for example)).

In certain embodiments, the alpha chain comprises a variable and joining (VJ) region comprising an amino acid sequence that is at least 90%, 95%, 96%, 97%, 98%, 99% or 100% identical to an amino acid sequence provided in Table 4, and the corresponding beta chain comprises a variable, diversity and joining (VDJ) region comprising an amino acid sequence that is at least 90%, 95%, 96%, 97%, 98%, 99% or 100% identical to an amino acid sequence provided in Table 5. An alpha chain VJ region corresponds to a beta chain VDJ region if they appear in the same row in Table 4 and Table 5 (i.e. the beta chain VDJ region provided in Table 5 is in the same row number (row 1, for example) as the alpha chain VJ region provided in Table 4 (row 1, for example)).

In certain embodiments, the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5 (i.e. the beta chain provided in Table 5 is in the same row number (row 1, for example) as the alpha chain provided in Table 4 (row 1, for example)).

Also described herein are nucleic acid molecules encoding the disclosed TCRs.

Also described herein are vectors comprising a disclosed nucleic acid molecule.

Further described provided are cells transformed to express any of the nucleic acid molecules described herein.

Still further described are cells comprising any of the disclosed vectors. In certain embodiments, the cell is a CD8+ T cell.

Also provided are pharmaceutical compositions comprising any of the disclosed TCRs, any of the disclosed nucleic acid molecules, any of the disclosed vectors, or any of the disclosed cells.

### DETAILED DESCRIPTION OF ILLUSTRATIVE EMBODIMENTS

The disclosed T-cell receptors (TCRs), polynucleotides, vectors, compositions, kits, and cells may be understood more readily by reference to the following detailed description, which forms a part of this disclosure. It is to be understood that the disclosed TCRs, polynucleotides, vectors, compositions, kits, and cells are not limited to those specifically described and/or shown herein, and that the terminology used herein is for the purpose of describing particular embodiments by way of example only and is not intended to be limiting of the claimed TCRs, polynucleotides, vectors, compositions, kits, and cells.

Unless specifically stated otherwise, any description as to a possible mechanism or mode of action or reason for improvement is meant to be illustrative only, and the disclosed TCRs, polynucleotides, vectors, compositions, kits, and cells are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value unless the context clearly dictates otherwise.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value.

If not otherwise specified, the term “about” signifies a variance of ±10% of the associated value. Thus, the term “about” is used to encompass variations of ±10% or less, variations of ±5% or less, variations of ±1% or less, variations of ±0.5% or less, or variations of ±0.10% or less from the specified value.

When a list is presented, unless stated otherwise, it is to be understood that each individual element of that list, and every combination of that list, is a separate embodiment. For example, a list of embodiments presented as “A, B, or C” is to be interpreted as including the embodiments, “A”, “B”, “C”, “A or B”, “A or C”, “B or C”, or “A, B, or C”.

As used herein, the singular forms “a”, “an”, and “the” include the plural.

As used herein, the term “at least one” means “one or more.”

The terms “kit” and “article of manufacture” are used as synonyms.

“9-mer” or “9mer” refers to a polypeptide that is nine amino acids in length.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

### T-Cell Receptors

Disclosed herein are TCRs that bind to CALR and JAK2 epitopes. In some embodiments, the TCRs bind to a JAK2 epitope comprising the amino acid sequence of VLNYGVCFC (SEQ ID NO: 681). In some embodiments, the TCRs bind to a JAK2 epitope comprising the amino acid sequence of FCGDENILV (SEQ ID NO: 682). In some embodiments, the TCRs bind to a CALR epitope contained within the amino acid sequence MKDKQDEEQRTRRMMRTKMRMRRMRRTRRKMRRKMSPARPRTSCREACLQGWT EAAYEEAEDNCRRMMRTKAAYVLNYGVCFCAAYFCGDENILV (SEQ ID NO: 683), wherein the mutant CALR type 1 sequence is underlined, the mutant CALR type II sequence is underlined and bold, and the mutant JAK2 sequences are underlined an italics. In some embodiments, the TCRs bind to a mutJAK2 and mutCALR antigen construct comprising an amino acid sequence of SEQ ID NO: 702.

The disclosed TCRs may be identified based on T-cell binding to the CALR and JAK2 epitopes, followed by sequencing of the TCR. The TCRs may be obtained from ap T cells. The TCRs may be further engineered to improve their affinity, stability, solubility, or the like. For example, TCRs may be cysteine stabilized, expressed as soluble TCRs, as single chain TCRs, as a fusion with N-terminal or C-terminal epitope tags, engineered to improve stability with mutations in hydrophobic core, such as positions 11, 13, 19, 21, 53, 76, 89, 91 or 94 of the α chain, or domain swapped with α and β chain variable and/or constant domains swapped as described in U.S. Pat. Nos. 7,329,731, 7,569,664, 9,133,264, 9,624,292, US2016/0130319 and U.S. Pat. No. 9,884,075.

Described herein are TCRs comprising an alpha chain and a beta chain, wherein the alpha chain comprises a CDR3 comprising an amino acid sequence provided in Table 4, and the beta chain comprises a CDR3 comprising a corresponding amino acid sequence provided in Table 5. (i.e. the beta chain CDR3 provided in Table 5 is in the same row number (row 1, for example) as the alpha chain CDR3 provided in Table 4 (row 1, for example)).

Also described herein are TCRs comprising an alpha chain and a beta chain, wherein the alpha chain comprises a CDR1 comprising an amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5 (i.e. the beta chain CDRs provided in Table 5 are in the same row number (row 1, for example) as the alpha chain CDRs provided in Table 4 (row 1, for example)).

In certain embodiments, the alpha chain comprises a variable and joining (VJ) region comprising an amino acid sequence that is at least 90%, 95%, 96%, 97%, 98%, 99% or 100% identical to an amino acid sequence provided in Table 4, and the corresponding beta chain comprises a variable, diversity and joining (VDJ) region comprising an amino acid sequence that is at least 90%, 95%, 96%, 97%, 98%, 99% or 100% identical to an amino acid sequence provided in Table 5 (i.e. the beta chain VDJ region provided in Table 5 is in the same row number (row 1, for example) as the alpha chain VJ region provided in Table 4 (row 1, for example)).

In certain embodiments, the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5 (i.e. the beta chain provided in Table 5 is in the same row number (row 1, for example) as the alpha chain provided in Table 4 (row 1, for example)).

Described herein is a TCR comprising an alpha chain and a beta chain, wherein the alpha chain comprises a CDR3 comprising the amino acid sequence of SEQ ID NO: 7, and the beta chain comprises a CDR3 comprising the amino acid sequence of SEQ ID NO: 245. Also described herein is a TCR comprising an alpha chain and a beta chain, wherein the alpha chain comprises a CDR1 comprising the amino acid sequence of SEQ ID NO: 3, a CDR2 comprising the amino acid sequence of SEQ ID NO: 5, and a CDR3 comprising the amino acid sequence of SEQ ID NO: 7, and the beta chain comprises a CDR1 comprising the amino acid sequence of SEQ ID NO: 241, a CDR2 comprising the amino acid sequence of SEQ ID NO: 243, and a CDR3 comprising the amino acid sequence of SEQ ID NO: 245. In certain embodiments, the alpha chain comprises a VJ region comprising an amino acid sequence that is at least 90%, 95%, 96%, 97%, 98%, 99% or 100% identical to the amino acid sequence of SEQ ID NO: 479, and the beta chain comprises a VDJ region comprising an amino acid sequence that is at least 90%, 95%, 96%, 97%, 98%, 99% or 100% identical to the amino acid sequence of SEQ ID NO: 581. In certain embodiments, the alpha chain comprises an amino acid sequence that is at least 90%, 95%, 96%, 97%, 98%, 99% or 100% identical to the amino acid sequence of SEQ ID NO: 477, and the beta chain comprises an amino acid sequence that is at least 90%, 95%, 96%, 97%, 98%, 99% or 100% identical to the amino acid sequence of SEQ ID NO: 579.

Described herein is a TCR comprising an alpha chain and a beta chain, wherein the alpha chain comprises a CDR3 comprising the amino acid sequence of SEQ ID NO: 14, and the beta chain comprises a CDR3 comprising the amino acid sequence of SEQ ID NO: 252. Also described herein is a TCR comprising an alpha chain and a beta chain, wherein the alpha chain comprises a CDR1 comprising the amino acid sequence of SEQ ID NO: 10, a CDR2 comprising the amino acid sequence of SEQ ID NO: 12, and a CDR3 comprising the amino acid sequence of SEQ ID NO: 14, and the beta chain comprises a CDR1 comprising the amino acid sequence of SEQ ID NO: 248, a CDR2 comprising the amino acid sequence of SEQ ID NO: 250, and a CDR3 comprising the amino acid sequence of SEQ ID NO: 252. In certain embodiments, the alpha chain comprises a VJ region comprising an amino acid sequence that is at least 90%, 95%, 96%, 97%, 98%, 99% or 100% identical to the amino acid sequence of SEQ ID NO: 482, and the beta chain comprises a VJD region comprising an amino acid sequence that is at least 90%, 95%, 96%, 97%, 98%, 99% or 100% identical to the amino acid sequence of SEQ ID NO: 584. In certain embodiments, the alpha chain comprises an amino acid sequence that is at least 90%, 95%, 96%, 97%, 98%, 99% or 100% identical to the amino acid sequence of SEQ ID NO: 480, and the beta chain comprises an amino acid sequence that is at least 90%, 95%, 96%, 97%, 98%, 99% or 100% identical to the amino acid sequence of SEQ ID NO: 582.

Described herein is a TCR comprising an alpha chain and a beta chain, wherein the alpha chain comprises a CDR3 comprising the amino acid sequence of SEQ ID NO: 21, and the beta chain comprises a CDR3 comprising the amino acid sequence of SEQ ID NO: 259. Also described herein is a TCR comprising an alpha chain and a beta chain, wherein the alpha chain comprises a CDR1 comprising the amino acid sequence of SEQ ID NO: 17, a CDR2 comprising the amino acid sequence of SEQ ID NO: 19, and a CDR3 comprising the amino acid sequence of SEQ ID NO: 21, and the beta chain comprises a CDR1 comprising the amino acid sequence of SEQ ID NO: 255, a CDR2 comprising the amino acid sequence of SEQ ID NO: 257, and a CDR3 comprising the amino acid sequence of SEQ ID NO: 259. In certain embodiments, the alpha chain comprises a VJ region comprising an amino acid sequence that is at least 90%, 95%, 96%, 97%, 98%, 99% or 100% identical to the amino acid sequence of SEQ ID NO: 485, and the beta chain comprises a VJD region comprising an amino acid sequence that is at least 90%, 95%, 96%, 97%, 98%, 99% or 100% identical to the amino acid sequence of SEQ ID NO: 587. In certain embodiments, the alpha chain comprises an amino acid sequence that is at least 90%, 95%, 96%, 97%, 98%, 99% or 100% identical to the amino acid sequence of SEQ ID NO: 483, and the beta chain comprises an amino acid sequence that is at least 90%, 95%, 96%, 97%, 98%, 99% or 100% identical to the amino acid sequence of SEQ ID NO: 585.

Described herein is a TCR comprising an alpha chain and a beta chain, wherein the alpha chain comprises a CDR3 comprising the amino acid sequence of SEQ ID NO: 28, and the beta chain comprises a CDR3 comprising the amino acid sequence of SEQ ID NO: 266. Also described herein is a TCR comprising an alpha chain and a beta chain, wherein the alpha chain comprises a CDR1 comprising the amino acid sequence of SEQ ID NO: 24, a CDR2 comprising the amino acid sequence of SEQ ID NO: 26, and a CDR3 comprising the amino acid sequence of SEQ ID NO: 28, and the beta chain comprises a CDR1 comprising the amino acid sequence of SEQ ID NO: 262, a CDR2 comprising the amino acid sequence of SEQ ID NO: 264, and a CDR3 comprising the amino acid sequence of SEQ ID NO: 266. In certain embodiments, the alpha chain comprises a VJ region comprising an amino acid sequence that is at least 90%, 95%, 96%, 97%, 98%, 99% or 100% identical to the amino acid sequence of SEQ ID NO: 488, and the beta chain comprises a VJD region comprising an amino acid sequence that is at least 90%, 95%, 96%, 97%, 98%, 99% or 100% identical to the amino acid sequence of SEQ ID NO: 590. In certain embodiments, the alpha chain comprises an amino acid sequence that is at least 90%, 95%, 96%, 97%, 98%, 99% or 100% identical to the amino acid sequence of SEQ ID NO: 486, and the beta chain comprises an amino acid sequence that is at least 90%, 95%, 96%, 97%, 98%, 99% or 100% identical to the amino acid sequence of SEQ ID NO: 588.

Described herein is a TCR comprising an alpha chain and a beta chain, wherein the alpha chain comprises a CDR3 comprising the amino acid sequence of SEQ ID NO: 35, and the beta chain comprises a CDR3 comprising the amino acid sequence of SEQ ID NO: 273. Also described herein is a TCR comprising an alpha chain and a beta chain, wherein the alpha chain comprises a CDR1 comprising the amino acid sequence of SEQ ID NO: 31, a CDR2 comprising the amino acid sequence of SEQ ID NO: 33, and a CDR3 comprising the amino acid sequence of SEQ ID NO: 35, and the beta chain comprises a CDR1 comprising the amino acid sequence of SEQ ID NO: 269, a CDR2 comprising the amino acid sequence of SEQ ID NO: 271, and a CDR3 comprising the amino acid sequence of SEQ ID NO: 273. In certain embodiments, the alpha chain comprises a VJ region comprising an amino acid sequence that is at least 90%, 95%, 96%, 97%, 98%, 99% or 100% identical to the amino acid sequence of SEQ ID NO: 491, and the beta chain comprises a VJD region comprising an amino acid sequence that is at least 90%, 95%, 96%, 97%, 98%, 99% or 100% identical to the amino acid sequence of SEQ ID NO: 593. In certain embodiments, the alpha chain comprises an amino acid sequence that is at least 90%, 95%, 96%, 97%, 98%, 99% or 100% identical to the amino acid sequence of SEQ ID NO: 489, and the beta chain comprises an amino acid sequence that is at least 90%, 95%, 96%, 97%, 98%, 99% or 100% identical to the amino acid sequence of SEQ ID NO: 591.

Described herein is a TCR comprising an alpha chain and a beta chain, wherein the alpha chain comprises a CDR3 comprising the amino acid sequence of SEQ ID NO: 42, and the beta chain comprises a CDR3 comprising the amino acid sequence of SEQ ID NO: 280. Also described herein is a TCR comprising an alpha chain and a beta chain, wherein the alpha chain comprises a CDR1 comprising the amino acid sequence of SEQ ID NO: 38, a CDR2 comprising the amino acid sequence of SEQ ID NO: 40, and a CDR3 comprising the amino acid sequence of SEQ ID NO: 42, and the beta chain comprises a CDR1 comprising the amino acid sequence of SEQ ID NO: 276, a CDR2 comprising the amino acid sequence of SEQ ID NO: 278, and a CDR3 comprising the amino acid sequence of SEQ ID NO: 280. In certain embodiments, the alpha chain comprises a VJ region comprising an amino acid sequence that is at least 90%, 95%, 96%, 97%, 98%, 99% or 100% identical to the amino acid sequence of SEQ ID NO: 494, and the beta chain comprises a VJD region comprising an amino acid sequence that is at least 90%, 95%, 96%, 97%, 98%, 99% or 100% identical to the amino acid sequence of SEQ ID NO: 596. In certain embodiments, the alpha chain comprises an amino acid sequence that is at least 90%, 95%, 96%, 97%, 98%, 99% or 100% identical to the amino acid sequence of SEQ ID NO: 492, and the beta chain comprises an amino acid sequence that is at least 90%, 95%, 96%, 97%, 98%, 99% or 100% identical to the amino acid sequence of SEQ ID NO: 594.

Described herein is a TCR comprising an alpha chain and a beta chain, wherein the alpha chain comprises a CDR3 comprising the amino acid sequence of SEQ ID NO: 49, and the beta chain comprises a CDR3 comprising the amino acid sequence of SEQ ID NO: 287. Also described herein is a TCR comprising an alpha chain and a beta chain, wherein the alpha chain comprises a CDR1 comprising the amino acid sequence of SEQ ID NO: 45, a CDR2 comprising the amino acid sequence of SEQ ID NO: 47, and a CDR3 comprising the amino acid sequence of SEQ ID NO: 49, and the beta chain comprises a CDR1 comprising the amino acid sequence of SEQ ID NO: 283, a CDR2 comprising the amino acid sequence of SEQ ID NO: 285, and a CDR3 comprising the amino acid sequence of SEQ ID NO: 287. In certain embodiments, the alpha chain comprises a VJ region comprising an amino acid sequence that is at least 90%, 95%, 96%, 97%, 98%, 99% or 100% identical to the amino acid sequence of SEQ ID NO: 497, and the beta chain comprises a VJD region comprising an amino acid sequence that is at least 90%, 95%, 96%, 97%, 98%, 99% or 100% identical to the amino acid sequence of SEQ ID NO: 599. In certain embodiments, the alpha chain comprises an amino acid sequence that is at least 90%, 95%, 96%, 97%, 98%, 99% or 100% identical to the amino acid sequence of SEQ ID NO: 495, and the beta chain comprises an amino acid sequence that is at least 90%, 95%, 96%, 97%, 98%, 99% or 100% identical to the amino acid sequence of SEQ ID NO: 597.

Described herein is a TCR comprising an alpha chain and a beta chain, wherein the alpha chain comprises a CDR3 comprising the amino acid sequence of SEQ ID NO: 56, and the beta chain comprises a CDR3 comprising the amino acid sequence of SEQ ID NO: 294. Also described herein is a TCR comprising an alpha chain and a beta chain, wherein the alpha chain comprises a CDR1 comprising the amino acid sequence of SEQ ID NO: 52, a CDR2 comprising the amino acid sequence of SEQ ID NO: 54, and a CDR3 comprising the amino acid sequence of SEQ ID NO: 56, and the beta chain comprises a CDR1 comprising the amino acid sequence of SEQ ID NO: 290, a CDR2 comprising the amino acid sequence of SEQ ID NO: 292, and a CDR3 comprising the amino acid sequence of SEQ ID NO: 294. In certain embodiments, the alpha chain comprises a VJ region comprising an amino acid sequence that is at least 90%, 95%, 96%, 97%, 98%, 99% or 100% identical to the amino acid sequence of SEQ ID NO: 500, and the beta chain comprises a VJD region comprising an amino acid sequence that is at least 90%, 95%, 96%, 97%, 98%, 99% or 100% identical to the amino acid sequence of SEQ ID NO: 602. In certain embodiments, the alpha chain comprises an amino acid sequence that is at least 90%, 95%, 96%, 97%, 98%, 99% or 100% identical to the amino acid sequence of SEQ ID NO: 498, and the beta chain comprises an amino acid sequence that is at least 90%, 95%, 96%, 97%, 98%, 99% or 100% identical to the amino acid sequence of SEQ ID NO: 600.

Described herein is a TCR comprising an alpha chain and a beta chain, wherein the alpha chain comprises a CDR3 comprising the amino acid sequence of SEQ ID NO: 63, and the beta chain comprises a CDR3 comprising the amino acid sequence of SEQ ID NO: 301. Also described herein is a TCR comprising an alpha chain and a beta chain, wherein the alpha chain comprises a CDR1 comprising the amino acid sequence of SEQ ID NO: 59, a CDR2 comprising the amino acid sequence of SEQ ID NO: 61, and a CDR3 comprising the amino acid sequence of SEQ ID NO: 63, and the beta chain comprises a CDR1 comprising the amino acid sequence of SEQ ID NO: 297, a CDR2 comprising the amino acid sequence of SEQ ID NO: 299, and a CDR3 comprising the amino acid sequence of SEQ ID NO: 301. In certain embodiments, the alpha chain comprises a VJ region comprising an amino acid sequence that is at least 90%, 95%, 96%, 97%, 98%, 99% or 100% identical to the amino acid sequence of SEQ ID NO: 503, and the beta chain comprises a VJD region comprising an amino acid sequence that is at least 90%, 95%, 96%, 97%, 98%, 99% or 100% identical to the amino acid sequence of SEQ ID NO: 605. In certain embodiments, the alpha chain comprises an amino acid sequence that is at least 90%, 95%, 96%, 97%, 98%, 99% or 100% identical to the amino acid sequence of SEQ ID NO: 501, and the beta chain comprises an amino acid sequence that is at least 90%, 95%, 96%, 97%, 98%, 99% or 100% identical to the amino acid sequence of SEQ ID NO: 603.

Described herein is a TCR comprising an alpha chain and a beta chain, wherein the alpha chain comprises a CDR3 comprising the amino acid sequence of SEQ ID NO: 70, and the beta chain comprises a CDR3 comprising the amino acid sequence of SEQ ID NO: 308. Also described herein is a TCR comprising an alpha chain and a beta chain, wherein the alpha chain comprises a CDR1 comprising the amino acid sequence of SEQ ID NO: 66, a CDR2 comprising the amino acid sequence of SEQ ID NO: 68, and a CDR3 comprising the amino acid sequence of SEQ ID NO: 70, and the beta chain comprises a CDR1 comprising the amino acid sequence of SEQ ID NO: 304, a CDR2 comprising the amino acid sequence of SEQ ID NO: 306, and a CDR3 comprising the amino acid sequence of SEQ ID NO: 308. In certain embodiments, the alpha chain comprises a VJ region comprising an amino acid sequence that is at least 90%, 95%, 96%, 97%, 98%, 99% or 100% identical to the amino acid sequence of SEQ ID NO: 506, and the beta chain comprises a VJD region comprising an amino acid sequence that is at least 90%, 95%, 96%, 97%, 98%, 99% or 100% identical to the amino acid sequence of SEQ ID NO: 608. In certain embodiments, the alpha chain comprises an amino acid sequence that is at least 90%, 95%, 96%, 97%, 98%, 99% or 100% identical to the amino acid sequence of SEQ ID NO: 504, and the beta chain comprises an amino acid sequence that is at least 90%, 95%, 96%, 97%, 98%, 99% or 100% identical to the amino acid sequence of SEQ ID NO: 606.

Described herein is a TCR comprising an alpha chain and a beta chain, wherein the alpha chain comprises a CDR3 comprising the amino acid sequence of SEQ ID NO: 77, and the beta chain comprises a CDR3 comprising the amino acid sequence of SEQ ID NO: 315. Also described herein is a TCR comprising an alpha chain and a beta chain, wherein the alpha chain comprises a CDR1 comprising the amino acid sequence of SEQ ID NO: 73, a CDR2 comprising the amino acid sequence of SEQ ID NO: 75, and a CDR3 comprising the amino acid sequence of SEQ ID NO: 77, and the beta chain comprises a CDR1 comprising the amino acid sequence of SEQ ID NO: 311, a CDR2 comprising the amino acid sequence of SEQ ID NO: 313, and a CDR3 comprising the amino acid sequence of SEQ ID NO: 315. In certain embodiments, the alpha chain comprises a VJ region comprising an amino acid sequence that is at least 90%, 95%, 96%, 97%, 98%, 99% or 100% identical to the amino acid sequence of SEQ ID NO: 509, and the beta chain comprises a VJD region comprising an amino acid sequence that is at least 90%, 95%, 96%, 97%, 98%, 99% or 100% identical to the amino acid sequence of SEQ ID NO: 611. In certain embodiments, the alpha chain comprises an amino acid sequence that is at least 90%, 95%, 96%, 97%, 98%, 99% or 100% identical to the amino acid sequence of SEQ ID NO: 507, and the beta chain comprises an amino acid sequence that is at least 90%, 95%, 96%, 97%, 98%, 99% or 100% identical to the amino acid sequence of SEQ ID NO: 609.

Described herein is a TCR comprising an alpha chain and a beta chain, wherein the alpha chain comprises a CDR3 comprising the amino acid sequence of SEQ ID NO: 84, and the beta chain comprises a CDR3 comprising the amino acid sequence of SEQ ID NO: 322. Also described herein is a TCR comprising an alpha chain and a beta chain, wherein the alpha chain comprises a CDR1 comprising the amino acid sequence of SEQ ID NO: 80, a CDR2 comprising the amino acid sequence of SEQ ID NO: 82, and a CDR3 comprising the amino acid sequence of SEQ ID NO: 84, and the beta chain comprises a CDR1 comprising the amino acid sequence of SEQ ID NO: 318, a CDR2 comprising the amino acid sequence of SEQ ID NO: 320, and a CDR3 comprising the amino acid sequence of SEQ ID NO: 322. In certain embodiments, the alpha chain comprises a VJ region comprising an amino acid sequence that is at least 90%, 95%, 96%, 97%, 98%, 99% or 100% identical to the amino acid sequence of SEQ ID NO: 512, and the beta chain comprises a VJD region comprising an amino acid sequence that is at least 90%, 95%, 96%, 97%, 98%, 99% or 100% identical to the amino acid sequence of SEQ ID NO: 614. In certain embodiments, the alpha chain comprises an amino acid sequence that is at least 90%, 95%, 96%, 97%, 98%, 99% or 100% identical to the amino acid sequence of SEQ ID NO: 510, and the beta chain comprises an amino acid sequence that is at least 90%, 95%, 96%, 97%, 98%, 99% or 100% identical to the amino acid sequence of SEQ ID NO: 612.

Described herein is a TCR comprising an alpha chain and a beta chain, wherein the alpha chain comprises a CDR3 comprising the amino acid sequence of SEQ ID NO: 91, and the beta chain comprises a CDR3 comprising the amino acid sequence of SEQ ID NO: 329. Also described herein is a TCR comprising an alpha chain and a beta chain, wherein the alpha chain comprises a CDR1 comprising the amino acid sequence of SEQ ID NO: 87, a CDR2 comprising the amino acid sequence of SEQ ID NO: 89, and a CDR3 comprising the amino acid sequence of SEQ ID NO: 91, and the beta chain comprises a CDR1 comprising the amino acid sequence of SEQ ID NO: 325, a CDR2 comprising the amino acid sequence of SEQ ID NO: 327, and a CDR3 comprising the amino acid sequence of SEQ ID NO: 329. In certain embodiments, the alpha chain comprises a VJ region comprising an amino acid sequence that is at least 90%, 95%, 96%, 97%, 98%, 99% or 100% identical to the amino acid sequence of SEQ ID NO: 515, and the beta chain comprises a VJD region comprising an amino acid sequence that is at least 90%, 95%, 96%, 97%, 98%, 99% or 100% identical to the amino acid sequence of SEQ ID NO: 617. In certain embodiments, the alpha chain comprises an amino acid sequence that is at least 90%, 95%, 96%, 97%, 98%, 99% or 100% identical to the amino acid sequence of SEQ ID NO: 513, and the beta chain comprises an amino acid sequence that is at least 90%, 95%, 96%, 97%, 98%, 99% or 100% identical to the amino acid sequence of SEQ ID NO: 615.

Described herein is a TCR comprising an alpha chain and a beta chain, wherein the alpha chain comprises a CDR3 comprising the amino acid sequence of SEQ ID NO: 98, and the beta chain comprises a CDR3 comprising the amino acid sequence of SEQ ID NO: 336. Also described herein is a TCR comprising an alpha chain and a beta chain, wherein the alpha chain comprises a CDR1 comprising the amino acid sequence of SEQ ID NO: 94, a CDR2 comprising the amino acid sequence of SEQ ID NO: 96, and a CDR3 comprising the amino acid sequence of SEQ ID NO: 98, and the beta chain comprises a CDR1 comprising the amino acid sequence of SEQ ID NO: 332, a CDR2 comprising the amino acid sequence of SEQ ID NO: 334, and a CDR3 comprising the amino acid sequence of SEQ ID NO: 336. In certain embodiments, the alpha chain comprises a VJ region comprising an amino acid sequence that is at least 90%, 95%, 96%, 97%, 98%, 99% or 100% identical to the amino acid sequence of SEQ ID NO: 518, and the beta chain comprises a VJD region comprising an amino acid sequence that is at least 90%, 95%, 96%, 97%, 98%, 99% or 100% identical to the amino acid sequence of SEQ ID NO: 620. In certain embodiments, the alpha chain comprises an amino acid sequence that is at least 90%, 95%, 96%, 97%, 98%, 99% or 100% identical to the amino acid sequence of SEQ ID NO: 516, and the beta chain comprises an amino acid sequence that is at least 90%, 95%, 96%, 97%, 98%, 99% or 100% identical to the amino acid sequence of SEQ ID NO: 618.

Described herein is a TCR comprising an alpha chain and a beta chain, wherein the alpha chain comprises a CDR3 comprising the amino acid sequence of SEQ ID NO: 105, and the beta chain comprises a CDR3 comprising the amino acid sequence of SEQ ID NO: 343. Also described herein is a TCR comprising an alpha chain and a beta chain, wherein the alpha chain comprises a CDR1 comprising the amino acid sequence of SEQ ID NO: 101, a CDR2 comprising the amino acid sequence of SEQ ID NO: 103, and a CDR3 comprising the amino acid sequence of SEQ ID NO: 105, and the beta chain comprises a CDR1 comprising the amino acid sequence of SEQ ID NO: 339, a CDR2 comprising the amino acid sequence of SEQ ID NO: 341, and a CDR3 comprising the amino acid sequence of SEQ ID NO: 343. In certain embodiments, the alpha chain comprises a VJ region comprising an amino acid sequence that is at least 90%, 95%, 96%, 97%, 98%, 99% or 100% identical to the amino acid sequence of SEQ ID NO: 521, and the beta chain comprises a VJD region comprising an amino acid sequence that is at least 90%, 95%, 96%, 97%, 98%, 99% or 100% identical to the amino acid sequence of SEQ ID NO: 623. In certain embodiments, the alpha chain comprises an amino acid sequence that is at least 90%, 95%, 96%, 97%, 98%, 99% or 100% identical to the amino acid sequence of SEQ ID NO: 519, and the beta chain comprises an amino acid sequence that is at least 90%, 95%, 96%, 97%, 98%, 99% or 100% identical to the amino acid sequence of SEQ ID NO: 621.

Described herein is a TCR comprising an alpha chain and a beta chain, wherein the alpha chain comprises a CDR3 comprising the amino acid sequence of SEQ ID NO: 112, and the beta chain comprises a CDR3 comprising the amino acid sequence of SEQ ID NO: 350. Also described herein is a TCR comprising an alpha chain and a beta chain, wherein the alpha chain comprises a CDR1 comprising the amino acid sequence of SEQ ID NO: 108, a CDR2 comprising the amino acid sequence of SEQ ID NO: 110, and a CDR3 comprising the amino acid sequence of SEQ ID NO: 112, and the beta chain comprises a CDR1 comprising the amino acid sequence of SEQ ID NO: 346, a CDR2 comprising the amino acid sequence of SEQ ID NO: 348, and a CDR3 comprising the amino acid sequence of SEQ ID NO: 350. In certain embodiments, the alpha chain comprises a VJ region comprising an amino acid sequence that is at least 90%, 95%, 96%, 97%, 98%, 99% or 100% identical to the amino acid sequence of SEQ ID NO: 524, and the beta chain comprises a VJD region comprising an amino acid sequence that is at least 90%, 95%, 96%, 97%, 98%, 99% or 100% identical to the amino acid sequence of SEQ ID NO: 626. In certain embodiments, the alpha chain comprises an amino acid sequence that is at least 90%, 95%, 96%, 97%, 98%, 99% or 100% identical to the amino acid sequence of SEQ ID NO: 522, and the beta chain comprises an amino acid sequence that is at least 90%, 95%, 96%, 97%, 98%, 99% or 100% identical to the amino acid sequence of SEQ ID NO: 624.

Described herein is a TCR comprising an alpha chain and a beta chain, wherein the alpha chain comprises a CDR3 comprising the amino acid sequence of SEQ ID NO: 119, and the beta chain comprises a CDR3 comprising the amino acid sequence of SEQ ID NO: 357. Also described herein is a TCR comprising an alpha chain and a beta chain, wherein the alpha chain comprises a CDR1 comprising the amino acid sequence of SEQ ID NO: 115, a CDR2 comprising the amino acid sequence of SEQ ID NO: 117, and a CDR3 comprising the amino acid sequence of SEQ ID NO: 119, and the beta chain comprises a CDR1 comprising the amino acid sequence of SEQ ID NO: 353, a CDR2 comprising the amino acid sequence of SEQ ID NO: 355, and a CDR3 comprising the amino acid sequence of SEQ ID NO: 357. In certain embodiments, the alpha chain comprises a VJ region comprising an amino acid sequence that is at least 90%, 95%, 96%, 97%, 98%, 99% or 100% identical to the amino acid sequence of SEQ ID NO: 527, and the beta chain comprises a VJD region comprising an amino acid sequence that is at least 90%, 95%, 96%, 97%, 98%, 99% or 100% identical to the amino acid sequence of SEQ ID NO: 629. In certain embodiments, the alpha chain comprises an amino acid sequence that is at least 90%, 95%, 96%, 97%, 98%, 99% or 100% identical to the amino acid sequence of SEQ ID NO: 525, and the beta chain comprises an amino acid sequence that is at least 90%, 95%, 96%, 97%, 98%, 99% or 100% identical to the amino acid sequence of SEQ ID NO: 627.

Described herein is a TCR comprising an alpha chain and a beta chain, wherein the alpha chain comprises a CDR3 comprising the amino acid sequence of SEQ ID NO: 126, and the beta chain comprises a CDR3 comprising the amino acid sequence of SEQ ID NO: 364. Also described herein is a TCR comprising an alpha chain and a beta chain, wherein the alpha chain comprises a CDR1 comprising the amino acid sequence of SEQ ID NO: 122, a CDR2 comprising the amino acid sequence of SEQ ID NO: 124, and a CDR3 comprising the amino acid sequence of SEQ ID NO: 126, and the beta chain comprises a CDR1 comprising the amino acid sequence of SEQ ID NO: 360, a CDR2 comprising the amino acid sequence of SEQ ID NO: 362, and a CDR3 comprising the amino acid sequence of SEQ ID NO: 364. In certain embodiments, the alpha chain comprises a VJ region comprising an amino acid sequence that is at least 90%, 95%, 96%, 97%, 98%, 99% or 100% identical to the amino acid sequence of SEQ ID NO: 530, and the beta chain comprises a VJD region comprising an amino acid sequence that is at least 90%, 95%, 96%, 97%, 98%, 99% or 100% identical to the amino acid sequence of SEQ ID NO: 632. In certain embodiments, the alpha chain comprises an amino acid sequence that is at least 90%, 95%, 96%, 97%, 98%, 99% or 100% identical to the amino acid sequence of SEQ ID NO: 528, and the beta chain comprises an amino acid sequence that is at least 90%, 95%, 96%, 97%, 98%, 99% or 100% identical to the amino acid sequence of SEQ ID NO: 630.

Described herein is a TCR comprising an alpha chain and a beta chain, wherein the alpha chain comprises a CDR3 comprising the amino acid sequence of SEQ ID NO: 133, and the beta chain comprises a CDR3 comprising the amino acid sequence of SEQ ID NO: 371. Also described herein is a TCR comprising an alpha chain and a beta chain, wherein the alpha chain comprises a CDR1 comprising the amino acid sequence of SEQ ID NO: 129, a CDR2 comprising the amino acid sequence of SEQ ID NO: 131, and a CDR3 comprising the amino acid sequence of SEQ ID NO: 133, and the beta chain comprises a CDR1 comprising the amino acid sequence of SEQ ID NO: 367, a CDR2 comprising the amino acid sequence of SEQ ID NO: 369, and a CDR3 comprising the amino acid sequence of SEQ ID NO: 371. In certain embodiments, the alpha chain comprises a VJ region comprising an amino acid sequence that is at least 90%, 95%, 96%, 97%, 98%, 99% or 100% identical to the amino acid sequence of SEQ ID NO: 533, and the beta chain comprises a VJD region comprising an amino acid sequence that is at least 90%, 95%, 96%, 97%, 98%, 99% or 100% identical to the amino acid sequence of SEQ ID NO: 635. In certain embodiments, the alpha chain comprises an amino acid sequence that is at least 90%, 95%, 96%, 97%, 98%, 99% or 100% identical to the amino acid sequence of SEQ ID NO: 531, and the beta chain comprises an amino acid sequence that is at least 90%, 95%, 96%, 97%, 98%, 99% or 100% identical to the amino acid sequence of SEQ ID NO: 633.

Described herein is a TCR comprising an alpha chain and a beta chain, wherein the alpha chain comprises a CDR3 comprising the amino acid sequence of SEQ ID NO: 140, and the beta chain comprises a CDR3 comprising the amino acid sequence of SEQ ID NO: 378. Also described herein is a TCR comprising an alpha chain and a beta chain, wherein the alpha chain comprises a CDR1 comprising the amino acid sequence of SEQ ID NO: 136, a CDR2 comprising the amino acid sequence of SEQ ID NO: 138, and a CDR3 comprising the amino acid sequence of SEQ ID NO: 140, and the beta chain comprises a CDR1 comprising the amino acid sequence of SEQ ID NO: 374, a CDR2 comprising the amino acid sequence of SEQ ID NO: 376, and a CDR3 comprising the amino acid sequence of SEQ ID NO: 378. In certain embodiments, the alpha chain comprises a VJ region comprising an amino acid sequence that is at least 90%, 95%, 96%, 97%, 98%, 99% or 100% identical to the amino acid sequence of SEQ ID NO: 536, and the beta chain comprises a VJD region comprising an amino acid sequence that is at least 90%, 95%, 96%, 97%, 98%, 99% or 100% identical to the amino acid sequence of SEQ ID NO: 638. In certain embodiments, the alpha chain comprises an amino acid sequence that is at least 90%, 95%, 96%, 97%, 98%, 99% or 100% identical to the amino acid sequence of SEQ ID NO: 534, and the beta chain comprises an amino acid sequence that is at least 90%, 95%, 96%, 97%, 98%, 99% or 100% identical to the amino acid sequence of SEQ ID NO: 636.

Described herein is a TCR comprising an alpha chain and a beta chain, wherein the alpha chain comprises a CDR3 comprising the amino acid sequence of SEQ ID NO: 147, and the beta chain comprises a CDR3 comprising the amino acid sequence of SEQ ID NO: 385. Also described herein is a TCR comprising an alpha chain and a beta chain, wherein the alpha chain comprises a CDR1 comprising the amino acid sequence of SEQ ID NO: 143, a CDR2 comprising the amino acid sequence of SEQ ID NO: 145, and a CDR3 comprising the amino acid sequence of SEQ ID NO: 147, and the beta chain comprises a CDR1 comprising the amino acid sequence of SEQ ID NO: 381, a CDR2 comprising the amino acid sequence of SEQ ID NO: 383, and a CDR3 comprising the amino acid sequence of SEQ ID NO: 385. In certain embodiments, the alpha chain comprises a VJ region comprising an amino acid sequence that is at least 90%, 95%, 96%, 97%, 98%, 99% or 100% identical to the amino acid sequence of SEQ ID NO: 539, and the beta chain comprises a VJD region comprising an amino acid sequence that is at least 90%, 95%, 96%, 97%, 98%, 99% or 100% identical to the amino acid sequence of SEQ ID NO: 641. In certain embodiments, the alpha chain comprises an amino acid sequence that is at least 90%, 95%, 96%, 97%, 98%, 99% or 100% identical to the amino acid sequence of SEQ ID NO: 537, and the beta chain comprises an amino acid sequence that is at least 90%, 95%, 96%, 97%, 98%, 99% or 100% identical to the amino acid sequence of SEQ ID NO: 639.

Described herein is a TCR comprising an alpha chain and a beta chain, wherein the alpha chain comprises a CDR3 comprising the amino acid sequence of SEQ ID NO: 154, and the beta chain comprises a CDR3 comprising the amino acid sequence of SEQ ID NO: 392. Also described herein is a TCR comprising an alpha chain and a beta chain, wherein the alpha chain comprises a CDR1 comprising the amino acid sequence of SEQ ID NO: 150, a CDR2 comprising the amino acid sequence of SEQ ID NO: 152, and a CDR3 comprising the amino acid sequence of SEQ ID NO: 154, and the beta chain comprises a CDR1 comprising the amino acid sequence of SEQ ID NO: 388, a CDR2 comprising the amino acid sequence of SEQ ID NO: 390, and a CDR3 comprising the amino acid sequence of SEQ ID NO: 392. In certain embodiments, the alpha chain comprises a VJ region comprising an amino acid sequence that is at least 90%, 95%, 96%, 97%, 98%, 99% or 100% identical to the amino acid sequence of SEQ ID NO: 542, and the beta chain comprises a VJD region comprising an amino acid sequence that is at least 90%, 95%, 96%, 97%, 98%, 99% or 100% identical to the amino acid sequence of SEQ ID NO: 644. In certain embodiments, the alpha chain comprises an amino acid sequence that is at least 90%, 95%, 96%, 97%, 98%, 99% or 100% identical to the amino acid sequence of SEQ ID NO: 540, and the beta chain comprises an amino acid sequence that is at least 90%, 95%, 96%, 97%, 98%, 99% or 100% identical to the amino acid sequence of SEQ ID NO: 642.

Described herein is a TCR comprising an alpha chain and a beta chain, wherein the alpha chain comprises a CDR3 comprising the amino acid sequence of SEQ ID NO: 161, and the beta chain comprises a CDR3 comprising the amino acid sequence of SEQ ID NO: 399. Also described herein is a TCR comprising an alpha chain and a beta chain, wherein the alpha chain comprises a CDR1 comprising the amino acid sequence of SEQ ID NO: 157, a CDR2 comprising the amino acid sequence of SEQ ID NO: 159, and a CDR3 comprising the amino acid sequence of SEQ ID NO: 161, and the beta chain comprises a CDR1 comprising the amino acid sequence of SEQ ID NO: 395, a CDR2 comprising the amino acid sequence of SEQ ID NO: 397, and a CDR3 comprising the amino acid sequence of SEQ ID NO: 399. In certain embodiments, the alpha chain comprises a VJ region comprising an amino acid sequence that is at least 90%, 95%, 96%, 97%, 98%, 99% or 100% identical to the amino acid sequence of SEQ ID NO: 545, and the beta chain comprises a VJD region comprising an amino acid sequence that is at least 90%, 95%, 96%, 97%, 98%, 99% or 100% identical to the amino acid sequence of SEQ ID NO: 647. In certain embodiments, the alpha chain comprises an amino acid sequence that is at least 90%, 95%, 96%, 97%, 98%, 99% or 100% identical to the amino acid sequence of SEQ ID NO: 543, and the beta chain comprises an amino acid sequence that is at least 90%, 95%, 96%, 97%, 98%, 99% or 100% identical to the amino acid sequence of SEQ ID NO: 645.

Described herein is a TCR comprising an alpha chain and a beta chain, wherein the alpha chain comprises a CDR3 comprising the amino acid sequence of SEQ ID NO: 168, and the beta chain comprises a CDR3 comprising the amino acid sequence of SEQ ID NO: 406. Also described herein is a TCR comprising an alpha chain and a beta chain, wherein the alpha chain comprises a CDR1 comprising the amino acid sequence of SEQ ID NO: 164, a CDR2 comprising the amino acid sequence of SEQ ID NO: 166, and a CDR3 comprising the amino acid sequence of SEQ ID NO: 168, and the beta chain comprises a CDR1 comprising the amino acid sequence of SEQ ID NO: 402, a CDR2 comprising the amino acid sequence of SEQ ID NO: 404, and a CDR3 comprising the amino acid sequence of SEQ ID NO: 406. In certain embodiments, the alpha chain comprises a VJ region comprising an amino acid sequence that is at least 90%, 95%, 96%, 97%, 98%, 99% or 100% identical to the amino acid sequence of SEQ ID NO: 548, and the beta chain comprises a VJD region comprising an amino acid sequence that is at least 90%, 95%, 96%, 97%, 98%, 99% or 100% identical to the amino acid sequence of SEQ ID NO: 650. In certain embodiments, the alpha chain comprises an amino acid sequence that is at least 90%, 95%, 96%, 97%, 98%, 99% or 100% identical to the amino acid sequence of SEQ ID NO: 546, and the beta chain comprises an amino acid sequence that is at least 90%, 95%, 96%, 97%, 98%, 99% or 100% identical to the amino acid sequence of SEQ ID NO: 648.

Described herein is a TCR comprising an alpha chain and a beta chain, wherein the alpha chain comprises a CDR3 comprising the amino acid sequence of SEQ ID NO: 175, and the beta chain comprises a CDR3 comprising the amino acid sequence of SEQ ID NO: 413. Also described herein is a TCR comprising an alpha chain and a beta chain, wherein the alpha chain comprises a CDR1 comprising the amino acid sequence of SEQ ID NO: 171, a CDR2 comprising the amino acid sequence of SEQ ID NO: 173, and a CDR3 comprising the amino acid sequence of SEQ ID NO: 175, and the beta chain comprises a CDR1 comprising the amino acid sequence of SEQ ID NO: 409, a CDR2 comprising the amino acid sequence of SEQ ID NO: 411, and a CDR3 comprising the amino acid sequence of SEQ ID NO: 413. In certain embodiments, the alpha chain comprises a VJ region comprising an amino acid sequence that is at least 90%, 95%, 96%, 97%, 98%, 99% or 100% identical to the amino acid sequence of SEQ ID NO: 551, and the beta chain comprises a VJD region comprising an amino acid sequence that is at least 90%, 95%, 96%, 97%, 98%, 99% or 100% identical to the amino acid sequence of SEQ ID NO: 653. In certain embodiments, the alpha chain comprises an amino acid sequence that is at least 90%, 95%, 96%, 97%, 98%, 99% or 100% identical to the amino acid sequence of SEQ ID NO: 549, and the beta chain comprises an amino acid sequence that is at least 90%, 95%, 96%, 97%, 98%, 99% or 100% identical to the amino acid sequence of SEQ ID NO: 651.

Described herein is a TCR comprising an alpha chain and a beta chain, wherein, the alpha chain comprises a CDR3 comprising the amino acid sequence of SEQ ID NO: 182, and the beta chain comprises a CDR3 comprising the amino acid sequence of SEQ ID NO: 420. Also described herein is a TCR comprising an alpha chain and a beta chain, wherein the alpha chain comprises a CDR1 comprising the amino acid sequence of SEQ ID NO: 178, a CDR2 comprising the amino acid sequence of SEQ ID NO: 180, and a CDR3 comprising the amino acid sequence of SEQ ID NO: 182, and the beta chain comprises a CDR1 comprising the amino acid sequence of SEQ ID NO: 416, a CDR2 comprising the amino acid sequence of SEQ ID NO: 418, and a CDR3 comprising the amino acid sequence of SEQ ID NO: 420. In certain embodiments, the alpha chain comprises a VJ region comprising an amino acid sequence that is at least 90%, 95%, 96%, 97%, 98%, 99% or 100% identical to the amino acid sequence of SEQ ID NO: 554, and the beta chain comprises a VJD region comprising an amino acid sequence that is at least 90%, 95%, 96%, 97%, 98%, 99% or 100% identical to the amino acid sequence of SEQ ID NO: 656. In certain embodiments, the alpha chain comprises an amino acid sequence that is at least 90%, 95%, 96%, 97%, 98%, 99% or 100% identical to the amino acid sequence of SEQ ID NO: 552, and the beta chain comprises an amino acid sequence that is at least 90%, 95%, 96%, 97%, 98%, 99% or 100% identical to the amino acid sequence of SEQ ID NO: 654.

Described herein is a TCR comprising an alpha chain and a beta chain, wherein the alpha chain comprises a CDR3 comprising the amino acid sequence of SEQ ID NO: 189, and the beta chain comprises a CDR3 comprising the amino acid sequence of SEQ ID NO: 427. Also described herein is a TCR comprising an alpha chain and a beta chain, wherein the alpha chain comprises a CDR1 comprising the amino acid sequence of SEQ ID NO: 185, a CDR2 comprising the amino acid sequence of SEQ ID NO: 187, and a CDR3 comprising the amino acid sequence of SEQ ID NO: 189, and the beta chain comprises a CDR1 comprising the amino acid sequence of SEQ ID NO: 423, a CDR2 comprising the amino acid sequence of SEQ ID NO: 425, and a CDR3 comprising the amino acid sequence of SEQ ID NO: 427. In certain embodiments, the alpha chain comprises a VJ region comprising an amino acid sequence that is at least 90%, 95%, 96%, 97%, 98%, 99% or 100% identical to the amino acid sequence of SEQ ID NO: 557, and the beta chain comprises a VJD region comprising an amino acid sequence that is at least 90%, 95%, 96%, 97%, 98%, 99% or 100% identical to the amino acid sequence of SEQ ID NO: 659. In certain embodiments, the alpha chain comprises an amino acid sequence that is at least 90%, 95%, 96%, 97%, 98%, 99% or 100% identical to the amino acid sequence of SEQ ID NO: 555, and the beta chain comprises an amino acid sequence that is at least 90%, 95%, 96%, 97%, 98%, 99% or 100% identical to the amino acid sequence of SEQ ID NO: 657.

Described herein is a TCR comprising an alpha chain and a beta chain, wherein, the alpha chain comprises a CDR3 comprising the amino acid sequence of SEQ ID NO: 196, and the beta chain comprises a CDR3 comprising the amino acid sequence of SEQ ID NO: 434. Also described herein is a TCR comprising an alpha chain and a beta chain, wherein the alpha chain comprises a CDR1 comprising the amino acid sequence of SEQ ID NO: 192, a CDR2 comprising the amino acid sequence of SEQ ID NO: 194, and a CDR3 comprising the amino acid sequence of SEQ ID NO: 196, and the beta chain comprises a CDR1 comprising the amino acid sequence of SEQ ID NO: 430, a CDR2 comprising the amino acid sequence of SEQ ID NO: 432, and a CDR3 comprising the amino acid sequence of SEQ ID NO: 434. In certain embodiments, the alpha chain comprises a VJ region comprising an amino acid sequence that is at least 90%, 95%, 96%, 97%, 98%, 99% or 100% identical to the amino acid sequence of SEQ ID NO: 560, and the beta chain comprises a VJD region comprising an amino acid sequence that is at least 90%, 95%, 96%, 97%, 98%, 99% or 100% identical to the amino acid sequence of SEQ ID NO: 662. In certain embodiments, the alpha chain comprises an amino acid sequence that is at least 90%, 95%, 96%, 97%, 98%, 99% or 100% identical to the amino acid sequence of SEQ ID NO: 558, and the beta chain comprises an amino acid sequence that is at least 90%, 95%, 96%, 97%, 98%, 99% or 100% identical to the amino acid sequence of SEQ ID NO: 660.

Described herein is a TCR comprising an alpha chain and a beta chain, wherein the alpha chain comprises a CDR3 comprising the amino acid sequence of SEQ ID NO: 203, and the beta chain comprises a CDR3 comprising the amino acid sequence of SEQ ID NO: 441. Also described herein is a TCR comprising an alpha chain and a beta chain, wherein the alpha chain comprises a CDR1 comprising the amino acid sequence of SEQ ID NO: 199, a CDR2 comprising the amino acid sequence of SEQ ID NO: 201, and a CDR3 comprising the amino acid sequence of SEQ ID NO: 203, and the beta chain comprises a CDR1 comprising the amino acid sequence of SEQ ID NO: 437, a CDR2 comprising the amino acid sequence of SEQ ID NO: 439, and a CDR3 comprising the amino acid sequence of SEQ ID NO: 441. In certain embodiments, the alpha chain comprises a VJ region comprising an amino acid sequence that is at least 90%, 95%, 96%, 97%, 98%, 99% or 100% identical to the amino acid sequence of SEQ ID NO: 563, and the beta chain comprises a VJD region comprising an amino acid sequence that is at least 90%, 95%, 96%, 97%, 98%, 99% or 100% identical to the amino acid sequence of SEQ ID NO: 665. In certain embodiments, the alpha chain comprises an amino acid sequence that is at least 90%, 95%, 96%, 97%, 98%, 99% or 100% identical to the amino acid sequence of SEQ ID NO: 561, and the beta chain of comprises an amino acid sequence that is at least 90%, 95%, 96%, 97%, 98%, 99% or 100% identical to the amino acid sequence of SEQ ID NO: 663.

Described herein is a TCR comprising an alpha chain and a beta chain, wherein the alpha chain comprises a CDR3 comprising the amino acid sequence of SEQ ID NO: 210, and the beta chain comprises a CDR3 comprising the amino acid sequence of SEQ ID NO: 448. Also described herein is a TCR comprising an alpha chain and a beta chain, wherein the alpha chain comprises a CDR1 comprising the amino acid sequence of SEQ ID NO: 206, a CDR2 comprising the amino acid sequence of SEQ ID NO: 208, and a CDR3 comprising the amino acid sequence of SEQ ID NO: 210, and the beta chain comprises a CDR1 comprising the amino acid sequence of SEQ ID NO: 444, a CDR2 comprising the amino acid sequence of SEQ ID NO: 446, and a CDR3 comprising the amino acid sequence of SEQ ID NO: 448. In certain embodiments, the alpha chain comprises a VJ region comprising an amino acid sequence that is at least 90%, 95%, 96%, 97%, 98%, 99% or 100% identical to the amino acid sequence of SEQ ID NO: 566, and the beta chain comprises a VJD region comprising an amino acid sequence that is at least 90%, 95%, 96%, 97%, 98%, 99% or 100% identical to the amino acid sequence of SEQ ID NO: 668. In certain embodiments, the alpha chain comprises an amino acid sequence that is at least 90%, 95%, 96%, 97%, 98%, 99% or 100% identical to the amino acid sequence of SEQ ID NO: 564, and the beta chain comprises an amino acid sequence that is at least 90%, 95%, 96%, 97%, 98%, 99% or 100% identical to the amino acid sequence of SEQ ID NO: 666.

Described herein is a TCR comprising an alpha chain and a beta chain, wherein the alpha chain comprises a CDR3 comprising the amino acid sequence of SEQ ID NO: 217, and the beta chain comprises a CDR3 comprising the amino acid sequence of SEQ ID NO: 455. Also described herein is a TCR comprising an alpha chain and a beta chain, wherein the alpha chain comprises a CDR1 comprising the amino acid sequence of SEQ ID NO: 213, a CDR2 comprising the amino acid sequence of SEQ ID NO: 215, and a CDR3 comprising the amino acid sequence of SEQ ID NO: 217, and the beta chain comprises a CDR1 comprising the amino acid sequence of SEQ ID NO: 451, a CDR2 comprising the amino acid sequence of SEQ ID NO: 453, and a CDR3 comprising the amino acid sequence of SEQ ID NO: 455. In certain embodiments, the alpha chain comprises a VJ region comprising an amino acid sequence that is at least 90%, 95%, 96%, 97%, 98%, 99% or 100% identical to the amino acid sequence of SEQ ID NO: 569, and the beta chain comprises a VJD region comprising an amino acid sequence that is at least 90%, 95%, 96%, 97%, 98%, 99% or 100% identical to the amino acid sequence of SEQ ID NO: 671. In certain embodiments, the alpha chain comprises an amino acid sequence that is at least 90%, 95%, 96%, 97%, 98%, 99% or 100% identical to the amino acid sequence of SEQ ID NO: 567, and the beta chain comprises an amino acid sequence that is at least 90%, 95%, 96%, 97%, 98%, 99% or 100% identical to the amino acid sequence of SEQ ID NO: 669.

Described herein is a TCR comprising an alpha chain and a beta chain, wherein the alpha chain comprises a CDR3 comprising the amino acid sequence of SEQ ID NO: 224, and the beta chain comprises a CDR3 comprising the amino acid sequence of SEQ ID NO: 462. Also described herein is a TCR comprising an alpha chain and a beta chain, wherein the alpha chain comprises a CDR1 comprising the amino acid sequence of SEQ ID NO: 220, a CDR2 comprising the amino acid sequence of SEQ ID NO: 222, and a CDR3 comprising the amino acid sequence of SEQ ID NO: 224, and the beta chain comprises a CDR1 comprising the amino acid sequence of SEQ ID NO: 458, a CDR2 comprising the amino acid sequence of SEQ ID NO: 460, and a CDR3 comprising the amino acid sequence of SEQ ID NO: 462. In certain embodiments, the alpha chain comprises a VJ region comprising an amino acid sequence that is at least 90%, 95%, 96%, 97%, 98%, 99% or 100% identical to the amino acid sequence of SEQ ID NO: 572, and the beta chain comprises a VJD region comprising an amino acid sequence that is at least 90%, 95%, 96%, 97%, 98%, 99% or 100% identical to the amino acid sequence of SEQ ID NO: 674. In certain embodiments, the alpha chain comprises an amino acid sequence that is at least 90%, 95%, 96%, 97%, 98%, 99% or 100% identical to the amino acid sequence of SEQ ID NO: 570, and the beta chain comprises an amino acid sequence that is at least 90%, 95%, 96%, 97%, 98%, 99% or 100% identical to the amino acid sequence of SEQ ID NO: 672.

Described herein is a TCR comprising an alpha chain and a beta chain, wherein the alpha chain comprises a CDR3 comprising the amino acid sequence of SEQ ID NO: 231, and the beta chain comprises a CDR3 comprising the amino acid sequence of SEQ ID NO: 469. Also described herein is a TCR comprising an alpha chain and a beta chain, wherein the alpha chain comprises a CDR1 comprising the amino acid sequence of SEQ ID NO: 227, a CDR2 comprising the amino acid sequence of SEQ ID NO: 229, and a CDR3 comprising the amino acid sequence of SEQ ID NO: 231, and the beta chain comprises a CDR1 comprising the amino acid sequence of SEQ ID NO: 465, a CDR2 comprising the amino acid sequence of SEQ ID NO: 467, and a CDR3 comprising the amino acid sequence of SEQ ID NO: 469. In certain embodiments, the alpha chain comprises a VJ region comprising an amino acid sequence that is at least 90%, 95%, 96%, 97%, 98%, 99% or 100% identical to the amino acid sequence of SEQ ID NO: 575, and the beta chain comprises a VJD region comprising an amino acid sequence that is at least 90%, 95%, 96%, 97%, 98%, 99% or 100% identical to the amino acid sequence of SEQ ID NO: 677. In certain embodiments, the alpha chain comprises an amino acid sequence that is at least 90%, 95%, 96%, 97%, 98%, 99% or 100% identical to the amino acid sequence of SEQ ID NO: 573, and the beta chain comprises an amino acid sequence that is at least 90%, 95%, 96%, 97%, 98%, 99% or 100% identical to the amino acid sequence of SEQ ID NO: 675.

Described herein is a TCR comprising an alpha chain and a beta chain, wherein, the alpha chain comprises a CDR3 comprising the amino acid sequence of SEQ ID NO: 238, and the beta chain comprises a CDR3 comprising the amino acid sequence of SEQ ID NO: 476. Also described herein is a TCR comprising an alpha chain and a beta chain, wherein the alpha chain comprises a CDR1 comprising the amino acid sequence of SEQ ID NO: 234, a CDR2 comprising the amino acid sequence of SEQ ID NO: 236, and a CDR3 comprising the amino acid sequence of SEQ ID NO: 238, and the beta chain comprises a CDR1 comprising the amino acid sequence of SEQ ID NO: 472, a CDR2 comprising the amino acid sequence of SEQ ID NO: 474, and a CDR3 comprising the amino acid sequence of SEQ ID NO: 476. In certain embodiments, the alpha chain comprises a VJ region comprising an amino acid sequence that is at least 90%, 95%, 96%, 97%, 98%, 99% or 100% identical to the amino acid sequence of SEQ ID NO: 578, and the beta chain comprises a VJD region comprising an amino acid sequence that is at least 90%, 95%, 96%, 97%, 98%, 99% or 100% identical to the amino acid sequence of SEQ ID NO: 680. In certain embodiments, the alpha chain comprises an amino acid sequence that is at least 90%, 95%, 96%, 97%, 98%, 99% or 100% identical to the amino acid sequence of SEQ ID NO: 576, and the beta chain comprises an amino acid sequence that is at least 90%, 95%, 96%, 97%, 98%, 99% or 100% identical to the amino acid sequence of SEQ ID NO: 678.

### Polynucleotides

The disclosure also provides polynucleotides that encode any of the disclosed TCRs.

Described herein are polynucleotides that encode a TCR comprising an alpha chain and a beta chain, wherein the alpha chain comprises a CDR3 comprising an amino acid sequence provided in Table 4, and the corresponding beta chain comprises a CDR3 comprising a corresponding amino acid sequence provided in Table 5. Described herein are polynucleotides that encode a TCR comprising an alpha chain and a beta chain, wherein the alpha chain comprises a CDR3 that is encoded by a nucleic acid sequence provided in Table 4, and the corresponding beta chain comprises a CDR3 that is encoded by a corresponding nucleic acid sequence provided in Table 5 (i.e. the beta chain CDR3 provided in Table 5 is in the same row number (row 1, for example) as the alpha chain CDR3 provided in Table 4 (row 1, for example)).

Also described herein are polynucleotides that encode a TCR comprising an alpha chain and a beta chain, wherein the alpha chain comprises a CDR1 comprising the amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5. Also described herein are polynucleotides that encode a TCR comprising an alpha chain and a beta chain, wherein the alpha chain comprises a CDR1 that is encoded by a nucleic acid sequence provided in Table 4, a CDR2 that is encoded by a nucleic acid sequence provided in Table 4, and a CDR3 that is encoded by a nucleic acid sequence provided in Table 4, and the corresponding beta chain comprises a CDR1 that is encoded by a nucleic acid sequence provided in Table 5, a CDR2 that is encoded by a nucleic acid sequence provided in Table 5, and a CDR3 that is encoded by a nucleic acid sequence provided in Table 5 (i.e. the beta chain CDRs provided in Table 5 are in the same row number (row 1, for example) as the alpha chain CDRs provided in Table 4 (row 1, for example)).

Also described herein are polynucleotides that encode a TCR comprising an alpha chain and a beta chain, wherein the alpha chain comprises a VJ region comprising an amino acid sequence that is at least 90%, 95%, 96%, 97%, 98%, 99% or 100% identical to an amino acid sequence provided in Table 4, and the corresponding beta chain comprises a VDJ region comprising an amino acid sequence that is at least 90%, 95%, 96%, 97%, 98%, 99% or 100% identical to an amino acid sequence provided in Table 5. Also described herein are polynucleotides that encode a TCR comprising an alpha chain and a beta chain, wherein the alpha chain comprises a VJ region that is encoded by a nucleic acid sequence that is at least 90%, 95%, 96%, 97%, 98%, 99% or 100% identical to a nucleic acid sequence provided in Table 4, and the corresponding beta chain comprises a VDJ region that is encoded by a nucleic acid sequence that is at least 90%, 95%, 96%, 97%, 98%, 99% or 100% identical to a nucleic acid sequence provided in Table 5 (i.e. the beta chain VDJ region provided in Table 5 is in the same row number (row 1, for example) as the alpha chain VJ region provided in Table 4 (row 1, for example)).

Also described herein are polynucleotides that encode a TCR comprising an alpha chain and a beta chain, wherein the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5. Also described herein are polynucleotides that encode a TCR comprising an alpha chain and a beta chain, wherein the alpha chain is encoded by a nucleic acid sequence that is at least 90%, 95%, 96%, 97%, 98%, 99% or 100% identical to a nucleic acid sequence provided in Table 4, and the corresponding beta chain is encoded by a nucleic acid sequence that is at least 90%, 95%, 96%, 97%, 98%, 99% or 100% identical to a nucleic acid sequence provided in Table 5 (i.e. the beta chain provided in Table 5 is in the same row number (row 1, for example) as the alpha chain provided in Table 4 (row 1, for example)).

Described herein are polynucleotides that encode a TCR comprising an alpha chain and a beta chain, wherein the alpha chain comprises a CDR3 that is encoded by the nucleic acid sequence of SEQ ID NO: 6, and the beta chain comprises a CDR3 that is encoded by the nucleic acid sequence of SEQ ID NO: 244. Also described herein are polynucleotides that encode a TCR comprising an alpha chain and a beta chain, wherein the alpha chain comprises a CDR1 that is encoded by the nucleic acid sequence of SEQ ID NO: 2, a CDR2 that is encoded by the nucleic acid sequence of SEQ ID NO: 4, and a CDR3 that is encoded by the nucleic acid sequence of SEQ ID NO: 6, and the beta chain comprises a CDR1 that is encoded by the nucleic acid sequence of SEQ ID NO: 240, a CDR2 that is encoded by the nucleic acid sequence of SEQ ID NO: 242, and a CDR3 that is encoded by the nucleic acid sequence of SEQ ID NO: 244. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 478, and the beta chain comprises a variable, diversity and joining (VDJ) region that is encoded by a nucleic acid sequence that is at least 90%, 95%, 96%, 97%, 98%, 99% or 100% identical to the nucleic acid sequence of SEQ ID NO: 580. In certain embodiments, the alpha chain is encoded by a nucleic acid sequence that is at least 90%, 95%, 96%, 97%, 98%, 99% or 100% identical to the nucleic acid sequence of SEQ ID NO: 1, and the beta chain is encoded by a nucleic acid sequence that is at least 90%, 95%, 96%, 97%, 98%, 99% or 100% identical to the nucleic acid sequence of SEQ ID NO: 239.

Described herein are polynucleotides that encode a TCR comprising an alpha chain and a beta chain, wherein the alpha chain comprises a CDR3 that is encoded by the nucleic acid sequence of SEQ ID NO: 13, and the beta chain comprises a CDR3 that is encoded by the nucleic acid sequence of SEQ ID NO: 251. Also described herein are polynucleotides that encode a TCR comprising an alpha chain and a beta chain, wherein the alpha chain comprises a CDR1 that is encoded by the nucleic acid sequence of SEQ ID NO: 9, a CDR2 that is encoded by the nucleic acid sequence of SEQ ID NO: 11, and a CDR3 that is encoded by the nucleic acid sequence of SEQ ID NO: 13, and the beta chain comprises a CDR1 that is encoded by the nucleic acid sequence of SEQ ID NO: 247, a CDR2 that is encoded by the nucleic acid sequence of SEQ ID NO: 249, and a CDR3 that is encoded by the nucleic acid sequence of SEQ ID NO: 251. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 481, and the beta chain comprises a VJD region that is encoded by a nucleic acid sequence that is at least 90%, 95%, 96%, 97%, 98%, 99% or 100% identical to the nucleic acid sequence of SEQ ID NO: 583. In certain embodiments, the alpha chain of is encoded by a nucleic acid sequence that is at least 90%, 95%, 96%, 97%, 98%, 99% or 100% identical to the nucleic acid sequence of SEQ ID NO: 8, and the beta chain is encoded by a nucleic acid sequence that is at least 90%, 95%, 96%, 97%, 98%, 99% or 100% identical to the nucleic acid sequence of SEQ ID NO: 246.

Described herein are polynucleotides that encode a TCR comprising an alpha chain and a beta chain, wherein the alpha chain comprises a CDR3 that is encoded by the nucleic acid sequence of SEQ ID NO: 20, and the beta chain comprises a CDR3 that is encoded by the nucleic acid sequence of SEQ ID NO: 258. Also described herein are polynucleotides that encode a TCR comprising an alpha chain and a beta chain, wherein the alpha chain comprises a CDR1 that is encoded by the nucleic acid sequence of SEQ ID NO: 16, a CDR2 that is encoded by the nucleic acid sequence of SEQ ID NO: 18, and a CDR3 that is encoded by the nucleic acid sequence of SEQ ID NO: 20, and the beta chain comprises a CDR1 that is encoded by the nucleic acid sequence of SEQ ID NO: 254, a CDR2 that is encoded by the nucleic acid sequence of SEQ ID NO: 256, and a CDR3 that is encoded by the nucleic acid sequence of SEQ ID NO: 258. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 484, and the beta chain comprises a VJD region that is encoded by a nucleic acid sequence that is at least 90%, 95%, 96%, 97%, 98%, 99% or 100% identical to the nucleic acid sequence of SEQ ID NO: 586. In certain embodiments, the alpha chain is encoded by a nucleic acid sequence that is at least 90%, 95%, 96%, 97%, 98%, 99% or 100% identical to the nucleic acid sequence of SEQ ID NO: 15, and the beta chain is encoded by a nucleic acid sequence that is at least 90%, 95%, 96%, 97%, 98%, 99% or 100% identical to the nucleic acid sequence of SEQ ID NO: 253.

Described herein are polynucleotides that encode a TCR comprising an alpha chain and a beta chain, wherein the alpha chain comprises a CDR3 that is encoded by the nucleic acid sequence of SEQ ID NO: 27, and the beta chain comprises a CDR3 that is encoded by the nucleic acid sequence of SEQ ID NO: 265. Also described herein are polynucleotides that encode a TCR comprising an alpha chain and a beta chain, wherein the alpha chain comprises a CDR1 that is encoded by the nucleic acid sequence of SEQ ID NO: 23, a CDR2 that is encoded by the nucleic acid sequence of SEQ ID NO: 25, and a CDR3 that is encoded by the nucleic acid sequence of SEQ ID NO: 27, and the beta chain comprises a CDR1 that is encoded by the nucleic acid sequence of SEQ ID NO: 261, a CDR2 that is encoded by the nucleic acid sequence of SEQ ID NO: 263, and a CDR3 that is encoded by the nucleic acid sequence of SEQ ID NO: 265. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 487, and the beta chain comprises a VJD region that is encoded by a nucleic acid sequence that is at least 90%, 95%, 96%, 97%, 98%, 99% or 100% identical to the nucleic acid sequence of SEQ ID NO: 589. In certain embodiments, the alpha chain is encoded by a nucleic acid sequence that is at least 90%, 95%, 96%, 97%, 98%, 99% or 100% identical to the nucleic acid sequence of SEQ ID NO: 22, and the beta chain is encoded by a nucleic acid sequence that is at least 90%, 95%, 96%, 97%, 98%, 99% or 100% identical to the nucleic acid sequence of SEQ ID NO: 260.

Described herein are polynucleotides that encode a TCR comprising an alpha chain and a beta chain, wherein the alpha chain comprises a CDR3 that is encoded by the nucleic acid sequence of SEQ ID NO: 34, and the beta chain comprises a CDR3 that is encoded by the nucleic acid sequence of SEQ ID NO: 272. Also described herein are polynucleotides that encode a TCR comprising an alpha chain and a beta chain, wherein the alpha chain comprises a CDR1 that is encoded by the nucleic acid sequence of SEQ ID NO: 30, a CDR2 that is encoded by the nucleic acid sequence of SEQ ID NO: 32, and a CDR3 that is encoded by the nucleic acid sequence of SEQ ID NO: 34, and the beta chain comprises a CDR1 that is encoded by the nucleic acid sequence of SEQ ID NO: 268, a CDR2 that is encoded by the nucleic acid sequence of SEQ ID NO: 270, and a CDR3 that is encoded by the nucleic acid sequence of SEQ ID NO: 272. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 490, and the beta chain comprises a VJD region that is encoded by a nucleic acid sequence that is at least 90%, 95%, 96%, 97%, 98%, 99% or 100% identical to the nucleic acid sequence of SEQ ID NO: 592. In certain embodiments, the alpha chain is encoded by a nucleic acid sequence that is at least 90%, 95%, 96%, 97%, 98%, 99% or 100% identical to the nucleic acid sequence of SEQ ID NO: 29, and the beta chain is encoded by a nucleic acid sequence that is at least 90%, 95%, 96%, 97%, 98%, 99% or 100% identical to the nucleic acid sequence of SEQ ID NO: 267.

Described herein are polynucleotides that encode a TCR comprising an alpha chain and a beta chain, wherein the alpha chain comprises a CDR3 that is encoded by the nucleic acid sequence of SEQ ID NO: 41, and the beta chain comprises a CDR3 that is encoded by the nucleic acid sequence of SEQ ID NO: 279. Also described herein are polynucleotides that encode a TCR comprising an alpha chain and a beta chain, wherein the alpha chain comprises a CDR1 that is encoded by the nucleic acid sequence of SEQ ID NO: 37, a CDR2 that is encoded by the nucleic acid sequence of SEQ ID NO: 39, and a CDR3 that is encoded by the nucleic acid sequence of SEQ ID NO: 41, and the beta chain comprises a CDR1 that is encoded by the nucleic acid sequence of SEQ ID NO: 275, a CDR2 that is encoded by the nucleic acid sequence of SEQ ID NO: 277, and a CDR3 that is encoded by the nucleic acid sequence of SEQ ID NO: 279. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 493, and the beta chain comprises a VJD region that is encoded by a nucleic acid sequence that is at least 90%, 95%, 96%, 97%, 98%, 99% or 100% identical to the nucleic acid sequence of SEQ ID NO: 595. In certain embodiments, the alpha chain is encoded by a nucleic acid sequence that is at least 90%, 95%, 96%, 97%, 98%, 99% or 100% identical to the nucleic acid sequence of SEQ ID NO: 36, and the beta chain is encoded by a nucleic acid sequence that is at least 90%, 95%, 96%, 97%, 98%, 99% or 100% identical to the nucleic acid sequence of SEQ ID NO: 274.

Described herein are polynucleotides that encode a TCR comprising an alpha chain and a beta chain, wherein the alpha chain comprises a CDR3 that is encoded by the nucleic acid sequence of SEQ ID NO: 48, and the beta chain comprises a CDR3 that is encoded by the nucleic acid sequence of SEQ ID NO: 286. Also described herein are polynucleotides that encode a TCR comprising an alpha chain and a beta chain, wherein the alpha chain comprises a CDR1 that is encoded by the nucleic acid sequence of SEQ ID NO: 44, a CDR2 that is encoded by the nucleic acid sequence of SEQ ID NO: 46, and a CDR3 that is encoded by the nucleic acid sequence of SEQ ID NO: 48, and the beta chain comprises a CDR1 that is encoded by the nucleic acid sequence of SEQ ID NO: 282, a CDR2 that is encoded by the nucleic acid sequence of SEQ ID NO: 284, and a CDR3 that is encoded by the nucleic acid sequence of SEQ ID NO: 286. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 496, and the beta chain comprises a VJD region that is encoded by a nucleic acid sequence that is at least 90%, 95%, 96%, 97%, 98%, 99% or 100% identical to the nucleic acid sequence of SEQ ID NO: 598. In certain embodiments, the alpha chain is encoded by a nucleic acid sequence that is at least 90%, 95%, 96%, 97%, 98%, 99% or 100% identical to the nucleic acid sequence of SEQ ID NO: 43, and the beta chain is encoded by a nucleic acid sequence that is at least 90%, 95%, 96%, 97%, 98%, 99% or 100% identical to the nucleic acid sequence of SEQ ID NO: 281.

Described herein are polynucleotides that encode a TCR comprising an alpha chain and a beta chain, wherein the alpha chain comprises a CDR3 that is encoded by the nucleic acid sequence of SEQ ID NO: 55, and the beta chain comprises a CDR3 that is encoded by the nucleic acid sequence of SEQ ID NO: 293. Also described herein are polynucleotides that encode a TCR comprising an alpha chain and a beta chain, wherein the alpha chain comprises a CDR1 that is encoded by the nucleic acid sequence of SEQ ID NO: 51, a CDR2 that is encoded by the nucleic acid sequence of SEQ ID NO: 53, and a CDR3 that is encoded by the nucleic acid sequence of SEQ ID NO: 55, and the beta chain comprises a CDR1 that is encoded by the nucleic acid sequence of SEQ ID NO: 289, a CDR2 that is encoded by the nucleic acid sequence of SEQ ID NO: 291, and a CDR3 that is encoded by the nucleic acid sequence of SEQ ID NO: 293. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 499, and the beta chain comprises a VJD region that is encoded by a nucleic acid sequence that is at least 90%, 95%, 96%, 97%, 98%, 99% or 100% identical to the nucleic acid sequence of SEQ ID NO: 601. In certain embodiments, the alpha chain is encoded by a nucleic acid sequence that is at least 90%, 95%, 96%, 97%, 98%, 99% or 100% identical to the nucleic acid sequence of SEQ ID NO: 50, and the beta chain is encoded by a nucleic acid sequence that is at least 90%, 95%, 96%, 97%, 98%, 99% or 100% identical to the nucleic acid sequence of SEQ ID NO: 288.

Described herein are polynucleotides that encode a TCR comprising an alpha chain and a beta chain, wherein the alpha chain comprises a CDR3 that is encoded by the nucleic acid sequence of SEQ ID NO: 62, and the beta chain comprises a CDR3 that is encoded by the nucleic acid sequence of SEQ ID NO: 300. Also described herein are polynucleotides that encode a TCR comprising an alpha chain and a beta chain, wherein the alpha chain comprises a CDR1 that is encoded by the nucleic acid sequence of SEQ ID NO: 58, a CDR2 that is encoded by the nucleic acid sequence of SEQ ID NO: 60, and a CDR3 that is encoded by the nucleic acid sequence of SEQ ID NO: 62, and the beta chain comprises a CDR1 that is encoded by the nucleic acid sequence of SEQ ID NO: 296, a CDR2 that is encoded by the nucleic acid sequence of SEQ ID NO: 298, and a CDR3 that is encoded by the nucleic acid sequence of SEQ ID NO: 300. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 502, and the beta chain comprises a VJD region that is encoded by a nucleic acid sequence that is at least 90%, 95%, 96%, 97%, 98%, 99% or 100% identical to the nucleic acid sequence of SEQ ID NO: 604. In certain embodiments, the alpha chain is encoded by a nucleic acid sequence that is at least 90%, 95%, 96%, 97%, 98%, 99% or 100% identical to the nucleic acid sequence of SEQ ID NO: 57, and the beta chain is encoded by a nucleic acid sequence that is at least 90%, 95%, 96%, 97%, 98%, 99% or 100% identical to the nucleic acid sequence of SEQ ID NO: 295.

Described herein are polynucleotides that encode a TCR comprising an alpha chain and a beta chain, wherein the alpha chain comprises a CDR3 that is encoded by the nucleic acid sequence of SEQ ID NO: 69, and the beta chain comprises a CDR3 that is encoded by the nucleic acid sequence of SEQ ID NO: 307. Also described herein are polynucleotides that encode a TCR comprising an alpha chain and a beta chain, wherein the alpha chain comprises a CDR1 that is encoded by the nucleic acid sequence of SEQ ID NO: 65, a CDR2 that is encoded by the nucleic acid sequence of SEQ ID NO: 67, and a CDR3 that is encoded by the nucleic acid sequence of SEQ ID NO: 69, and the beta chain comprises a CDR1 that is encoded by the nucleic acid sequence of SEQ ID NO: 303, a CDR2 that is encoded by the nucleic acid sequence of SEQ ID NO: 305, and a CDR3 that is encoded by the nucleic acid sequence of SEQ ID NO: 307. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 505, and the beta chain comprises a VJD region that is encoded by a nucleic acid sequence that is at least 90%, 95%, 96%, 97%, 98%, 99% or 100% identical to the nucleic acid sequence of SEQ ID NO: 607. In certain embodiments, the alpha chain is encoded by a nucleic acid sequence that is at least 90%, 95%, 96%, 97%, 98%, 99% or 100% identical to the nucleic acid sequence of SEQ ID NO: 64, and the beta chain is encoded by a nucleic acid sequence that is at least 90%, 95%, 96%, 97%, 98%, 99% or 100% identical to the nucleic acid sequence of SEQ ID NO: 302.

Described herein are polynucleotides that encode a TCR comprising an alpha chain and a beta chain, wherein the alpha chain comprises a CDR3 that is encoded by the nucleic acid sequence of SEQ ID NO: 76, and the beta chain comprises a CDR3 that is encoded by the nucleic acid sequence of SEQ ID NO: 314. Also described herein are polynucleotides that encode a TCR comprising an alpha chain and a beta chain, wherein the alpha chain comprises a CDR1 that is encoded by the nucleic acid sequence of SEQ ID NO: 72, a CDR2 that is encoded by the nucleic acid sequence of SEQ ID NO: 74, and a CDR3 that is encoded by the nucleic acid sequence of SEQ ID NO: 76, and the beta chain comprises a CDR1 that is encoded by the nucleic acid sequence of SEQ ID NO: 310, a CDR2 that is encoded by the nucleic acid sequence of SEQ ID NO: 312, and a CDR3 that is encoded by the nucleic acid sequence of SEQ ID NO: 314. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 508, and the beta chain comprises a VJD region that is encoded by a nucleic acid sequence that is at least 90%, 95%, 96%, 97%, 98%, 99% or 100% identical to the nucleic acid sequence of SEQ ID NO: 610. In certain embodiments, the alpha chain is encoded by a nucleic acid sequence that is at least 90%, 95%, 96%, 97%, 98%, 99% or 100% identical to the nucleic acid sequence of SEQ ID NO: 71, and the beta chain is encoded by a nucleic acid sequence that is at least 90%, 95%, 96%, 97%, 98%, 99% or 100% identical to the nucleic acid sequence of SEQ ID NO: 309.

Described herein are polynucleotides that encode a TCR comprising an alpha chain and a beta chain, wherein the alpha chain comprises a CDR3 that is encoded by the nucleic acid sequence of SEQ ID NO: 83, and the beta chain comprises a CDR3 that is encoded by the nucleic acid sequence of SEQ ID NO: 321. Also described herein are polynucleotides that encode a TCR comprising an alpha chain and a beta chain, wherein the alpha chain comprises a CDR1 that is encoded by the nucleic acid sequence of SEQ ID NO: 79, a CDR2 that is encoded by the nucleic acid sequence of SEQ ID NO: 81, and a CDR3 that is encoded by the nucleic acid sequence of SEQ ID NO: 83, and the beta chain comprises a CDR1 that is encoded by the nucleic acid sequence of SEQ ID NO: 317, a CDR2 that is encoded by the nucleic acid sequence of SEQ ID NO: 319, and a CDR3 that is encoded by the nucleic acid sequence of SEQ ID NO: 321. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 511, and the beta chain comprises a VJD region that is encoded by a nucleic acid sequence that is at least 90%, 95%, 96%, 97%, 98%, 99% or 100% identical to the nucleic acid sequence of SEQ ID NO: 613. In certain embodiments, the alpha chain is encoded by a nucleic acid sequence that is at least 90%, 95%, 96%, 97%, 98%, 99% or 100% identical to the nucleic acid sequence of SEQ ID NO: 78, and the beta chain is encoded by a nucleic acid sequence that is at least 90%, 95%, 96%, 97%, 98%, 99% or 100% identical to the nucleic acid sequence of SEQ ID NO: 316.

Described herein are polynucleotides that encode a TCR comprising an alpha chain and a beta chain, wherein the alpha chain comprises a CDR3 that is encoded by the nucleic acid sequence of SEQ ID NO: 90, and the beta chain comprises a CDR3 that is encoded by the nucleic acid sequence of SEQ ID NO: 328. Also described herein are polynucleotides that encode a TCR comprising an alpha chain and a beta chain, wherein the alpha chain comprises a CDR1 that is encoded by the nucleic acid sequence of SEQ ID NO: 86, a CDR2 that is encoded by the nucleic acid sequence of SEQ ID NO: 88, and a CDR3 that is encoded by the nucleic acid sequence of SEQ ID NO: 90, and the beta chain comprises a CDR1 that is encoded by the nucleic acid sequence of SEQ ID NO: 324, a CDR2 that is encoded by the nucleic acid sequence of SEQ ID NO: 326, and a CDR3 that is encoded by the nucleic acid sequence of SEQ ID NO: 328. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 514, and the beta chain comprises a VJD region that is encoded by a nucleic acid sequence that is at least 90%, 95%, 96%, 97%, 98%, 99% or 100% identical to the nucleic acid sequence of SEQ ID NO: 616. In certain embodiments, the alpha chain is encoded by a nucleic acid sequence that is at least 90%, 95%, 96%, 97%, 98%, 99% or 100% identical to the nucleic acid sequence of SEQ ID NO: 85, and the beta chain is encoded by a nucleic acid sequence that is at least 90%, 95%, 96%, 97%, 98%, 99% or 100% identical to the nucleic acid sequence of SEQ ID NO: 323.

Described herein are polynucleotides that encode a TCR comprising an alpha chain and a beta chain, wherein the alpha chain comprises a CDR3 that is encoded by the nucleic acid sequence of SEQ ID NO: 97, and the beta chain comprises a CDR3 that is encoded by the nucleic acid sequence of SEQ ID NO: 335. Also described herein are polynucleotides that encode a TCR comprising an alpha chain and a beta chain, wherein the alpha chain comprises a CDR1 that is encoded by the nucleic acid sequence of SEQ ID NO: 93, a CDR2 that is encoded by the nucleic acid sequence of SEQ ID NO: 95, and a CDR3 that is encoded by the nucleic acid sequence of SEQ ID NO: 97, and the beta chain comprises a CDR1 that is encoded by the nucleic acid sequence of SEQ ID NO: 331, a CDR2 that is encoded by the nucleic acid sequence of SEQ ID NO: 333, and a CDR3 that is encoded by the nucleic acid sequence of SEQ ID NO: 335. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 517, and the beta chain comprises a VJD region that is encoded by a nucleic acid sequence that is at least 90%, 95%, 96%, 97%, 98%, 99% or 100% identical to the nucleic acid sequence of SEQ ID NO: 619. In certain embodiments, the alpha chain is encoded by a nucleic acid sequence that is at least 90%, 95%, 96%, 97%, 98%, 99% or 100% identical to the nucleic acid sequence of SEQ ID NO: 92, and the beta chain is encoded by a nucleic acid sequence that is at least 90%, 95%, 96%, 97%, 98%, 99% or 100% identical to the nucleic acid sequence of SEQ ID NO: 330.

Described herein are polynucleotides that encode a TCR comprising an alpha chain and a beta chain, wherein the alpha chain comprises a CDR3 that is encoded by the nucleic acid sequence of SEQ ID NO: 104, and the beta chain comprises a CDR3 that is encoded by the nucleic acid sequence of SEQ ID NO: 342. Also described herein are polynucleotides that encode a TCR comprising an alpha chain and a beta chain, wherein the alpha chain comprises a CDR1 that is encoded by the nucleic acid sequence of SEQ ID NO: 100, a CDR2 that is encoded by the nucleic acid sequence of SEQ ID NO: 102, and a CDR3 that is encoded by the nucleic acid sequence of SEQ ID NO: 104, and the beta chain comprises a CDR1 that is encoded by the nucleic acid sequence of SEQ ID NO: 338, a CDR2 that is encoded by the nucleic acid sequence of SEQ ID NO: 340, and a CDR3 that is encoded by the nucleic acid sequence of SEQ ID NO: 342. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 520, and the beta chain comprises a VJD region that is encoded by a nucleic acid sequence that is at least 90%, 95%, 96%, 97%, 98%, 99% or 100% identical to the nucleic acid sequence of SEQ ID NO: 622. In certain embodiments, the alpha chain is encoded by a nucleic acid sequence that is at least 90%, 95%, 96%, 97%, 98%, 99% or 100% identical to the nucleic acid sequence of SEQ ID NO: 99, and the beta chain is encoded by a nucleic acid sequence that is at least 90%, 95%, 96%, 97%, 98%, 99% or 100% identical to the nucleic acid sequence of SEQ ID NO: 337.

Described herein are polynucleotides that encode a TCR comprising an alpha chain and a beta chain, wherein the alpha chain comprises a CDR3 that is encoded by the nucleic acid sequence of SEQ ID NO: 111, and the beta chain comprises a CDR3 that is encoded by the nucleic acid sequence of SEQ ID NO: 349. Also described herein are polynucleotides that encode a TCR comprising an alpha chain and a beta chain, wherein the alpha chain comprises a CDR1 that is encoded by the nucleic acid sequence of SEQ ID NO: 107, a CDR2 that is encoded by the nucleic acid sequence of SEQ ID NO: 109, and a CDR3 that is encoded by the nucleic acid sequence of SEQ ID NO: 111, and the beta chain comprises a CDR1 that is encoded by the nucleic acid sequence of SEQ ID NO: 345, a CDR2 that is encoded by the nucleic acid sequence of SEQ ID NO: 347, and a CDR3 that is encoded by the nucleic acid sequence of SEQ ID NO: 349. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 523, and the beta chain comprises a VJD region that is encoded by a nucleic acid sequence that is at least 90%, 95%, 96%, 97%, 98%, 99% or 100% identical to the nucleic acid sequence of SEQ ID NO: 625. In certain embodiments, the alpha chain is encoded by a nucleic acid sequence that is at least 90%, 95%, 96%, 97%, 98%, 99% or 100% identical to the nucleic acid sequence of SEQ ID NO: 106, and the beta chain is encoded by a nucleic acid sequence that is at least 90%, 95%, 96%, 97%, 98%, 99% or 100% identical to the nucleic acid sequence of SEQ ID NO: 344.

Described herein are polynucleotides that encode a TCR comprising an alpha chain and a beta chain, wherein the alpha chain comprises a CDR3 that is encoded by the nucleic acid sequence of SEQ ID NO: 118, and the beta chain comprises a CDR3 that is encoded by the nucleic acid sequence of SEQ ID NO: 356. Also described herein are polynucleotides that encode a TCR comprising an alpha chain and a beta chain, wherein the alpha chain comprises a CDR1 that is encoded by the nucleic acid sequence of SEQ ID NO: 114, a CDR2 that is encoded by the nucleic acid sequence of SEQ ID NO: 116, and a CDR3 that is encoded by the nucleic acid sequence of SEQ ID NO: 118, and the beta chain comprises a CDR1 that is encoded by the nucleic acid sequence of SEQ ID NO: 352, a CDR2 that is encoded by the nucleic acid sequence of SEQ ID NO: 354, and a CDR3 that is encoded by the nucleic acid sequence of SEQ ID NO: 356. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 526, and the beta chain comprises a VJD region that is encoded by a nucleic acid sequence that is at least 90%, 95%, 96%, 97%, 98%, 99% or 100% identical to the nucleic acid sequence of SEQ ID NO: 628. In certain embodiments, the alpha chain is encoded by a nucleic acid sequence that is at least 90%, 95%, 96%, 97%, 98%, 99% or 100% identical to the nucleic acid sequence of SEQ ID NO: 113, and the beta chain is encoded by a nucleic acid sequence that is at least 90%, 95%, 96%, 97%, 98%, 99% or 100% identical to the nucleic acid sequence of SEQ ID NO: 351.

Described herein are polynucleotides that encode a TCR comprising an alpha chain and a beta chain, wherein the alpha chain comprises a CDR3 that is encoded by the nucleic acid sequence of SEQ ID NO: 125, and the beta chain comprises a CDR3 that is encoded by the nucleic acid sequence of SEQ ID NO: 363. Also described herein are polynucleotides that encode a TCR comprising an alpha chain and a beta chain, wherein the alpha chain comprises a CDR1 that is encoded by the nucleic acid sequence of SEQ ID NO: 121, a CDR2 that is encoded by the nucleic acid sequence of SEQ ID NO: 123, and a CDR3 that is encoded by the nucleic acid sequence of SEQ ID NO: 125, and the beta chain comprises a CDR1 that is encoded by the nucleic acid sequence of SEQ ID NO: 359, a CDR2 that is encoded by the nucleic acid sequence of SEQ ID NO: 361, and a CDR3 that is encoded by the nucleic acid sequence of SEQ ID NO: 363. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 529, and the beta chain comprises a VJD region that is encoded by a nucleic acid sequence that is at least 90%, 95%, 96%, 97%, 98%, 99% or 100% identical to the nucleic acid sequence of SEQ ID NO: 631. In certain embodiments, the alpha chain is encoded by a nucleic acid sequence that is at least 90%, 95%, 96%, 97%, 98%, 99% or 100% identical to the nucleic acid sequence of SEQ ID NO: 120, and the beta chain is encoded by a nucleic acid sequence that is at least 90%, 95%, 96%, 97%, 98%, 99% or 100% identical to the nucleic acid sequence of SEQ ID NO: 358.

Described herein are polynucleotides that encode a TCR comprising an alpha chain and a beta chain, wherein the alpha chain comprises a CDR3 that is encoded by the nucleic acid sequence of SEQ ID NO: 132, and the beta chain comprises a CDR3 that is encoded by the nucleic acid sequence of SEQ ID NO: 370. Also described herein are polynucleotides that encode a TCR comprising an alpha chain and a beta chain, wherein the alpha chain comprises a CDR1 that is encoded by the nucleic acid sequence of SEQ ID NO: 128, a CDR2 that is encoded by the nucleic acid sequence of SEQ ID NO: 130, and a CDR3 that is encoded by the nucleic acid sequence of SEQ ID NO: 132, and the beta chain comprises a CDR1 that is encoded by the nucleic acid sequence of SEQ ID NO: 366, a CDR2 that is encoded by the nucleic acid sequence of SEQ ID NO: 368, and a CDR3 that is encoded by the nucleic acid sequence of SEQ ID NO: 370. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 532, and the beta chain comprises a VJD region that is encoded by a nucleic acid sequence that is at least 90%, 95%, 96%, 97%, 98%, 99% or 100% identical to the nucleic acid sequence of SEQ ID NO: 634. In certain embodiments, the alpha chain is encoded by a nucleic acid sequence that is at least 90%, 95%, 96%, 97%, 98%, 99% or 100% identical to the nucleic acid sequence of SEQ ID NO: 127, and the beta chain is encoded by a nucleic acid sequence that is at least 90%, 95%, 96%, 97%, 98%, 99% or 100% identical to the nucleic acid sequence of SEQ ID NO: 365.

Described herein are polynucleotides that encode a TCR comprising an alpha chain and a beta chain, wherein the alpha chain comprises a CDR3 that is encoded by the nucleic acid sequence of SEQ ID NO: 139, and the beta chain comprises a CDR3 that is encoded by the nucleic acid sequence of SEQ ID NO: 377. Also described herein are polynucleotides that encode a TCR comprising an alpha chain and a beta chain, wherein the alpha chain comprises a CDR1 that is encoded by the nucleic acid sequence of SEQ ID NO: 135, a CDR2 that is encoded by the nucleic acid sequence of SEQ ID NO: 137, and a CDR3 that is encoded by the nucleic acid sequence of SEQ ID NO: 139, and the beta chain comprises a CDR1 that is encoded by the nucleic acid sequence of SEQ ID NO: 373, a CDR2 that is encoded by the nucleic acid sequence of SEQ ID NO: 375, and a CDR3 that is encoded by the nucleic acid sequence of SEQ ID NO: 377. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 535, and the beta chain comprises a VJD region that is encoded by a nucleic acid sequence that is at least 90%, 95%, 96%, 97%, 98%, 99% or 100% identical to the nucleic acid sequence of SEQ ID NO: 637. In certain embodiments, the alpha chain is encoded by a nucleic acid sequence that is at least 90%, 95%, 96%, 97%, 98%, 99% or 100% identical to the nucleic acid sequence of SEQ ID NO: 134, and the beta chain is encoded by a nucleic acid sequence that is at least 90%, 95%, 96%, 97%, 98%, 99% or 100% identical to the nucleic acid sequence of SEQ ID NO: 372.

Described herein are polynucleotides that encode a TCR comprising an alpha chain and a beta chain, wherein the alpha chain comprises a CDR3 that is encoded by the nucleic acid sequence of SEQ ID NO: 146, and the beta chain comprises a CDR3 that is encoded by the nucleic acid sequence of SEQ ID NO: 384. Also described herein are polynucleotides that encode a TCR comprising an alpha chain and a beta chain, wherein the alpha chain comprises a CDR1 that is encoded by the nucleic acid sequence of SEQ ID NO: 142, a CDR2 that is encoded by the nucleic acid sequence of SEQ ID NO: 144, and a CDR3 that is encoded by the nucleic acid sequence of SEQ ID NO: 146, and the beta chain comprises a CDR1 that is encoded by the nucleic acid sequence of SEQ ID NO: 380, a CDR2 that is encoded by the nucleic acid sequence of SEQ ID NO: 382, and a CDR3 that is encoded by the nucleic acid sequence of SEQ ID NO: 384. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 538, and the beta chain comprises a VJD region that is encoded by a nucleic acid sequence that is at least 90%, 95%, 96%, 97%, 98%, 99% or 100% identical to the nucleic acid sequence of SEQ ID NO: 640. In certain embodiments, the alpha chain is encoded by a nucleic acid sequence that is at least 90%, 95%, 96%, 97%, 98%, 99% or 100% identical to the nucleic acid sequence of SEQ ID NO: 141, and the beta chain is encoded by a nucleic acid sequence that is at least 90%, 95%, 96%, 97%, 98%, 99% or 100% identical to the nucleic acid sequence of SEQ ID NO: 379.

Described herein are polynucleotides that encode a TCR comprising an alpha chain and a beta chain, wherein the alpha chain comprises a CDR3 that is encoded by the nucleic acid sequence of SEQ ID NO: 153, and the beta chain comprises a CDR3 that is encoded by the nucleic acid sequence of SEQ ID NO: 391. Also described herein are polynucleotides that encode a TCR comprising an alpha chain and a beta chain, wherein the alpha chain comprises a CDR1 that is encoded by the nucleic acid sequence of SEQ ID NO: 149, a CDR2 that is encoded by the nucleic acid sequence of SEQ ID NO: 151, and a CDR3 that is encoded by the nucleic acid sequence of SEQ ID NO: 153, and the beta chain comprises a CDR1 that is encoded by the nucleic acid sequence of SEQ ID NO: 387, a CDR2 that is encoded by the nucleic acid sequence of SEQ ID NO: 389, and a CDR3 that is encoded by the nucleic acid sequence of SEQ ID NO: 391. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 541, and the beta chain comprises a VJD region that is encoded by a nucleic acid sequence that is at least 90%, 95%, 96%, 97%, 98%, 99% or 100% identical to the nucleic acid sequence of SEQ ID NO: 643. In certain embodiments, the alpha chain is encoded by a nucleic acid sequence that is at least 90%, 95%, 96%, 97%, 98%, 99% or 100% identical to the nucleic acid sequence of SEQ ID NO: 148, and the beta chain is encoded by a nucleic acid sequence that is at least 90%, 95%, 96%, 97%, 98%, 99% or 100% identical to the nucleic acid sequence of SEQ ID NO: 386.

Described herein are polynucleotides that encode a TCR comprising an alpha chain and a beta chain, wherein the alpha chain comprises a CDR3 that is encoded by the nucleic acid sequence of SEQ ID NO: 160, and the beta chain comprises a CDR3 that is encoded by the nucleic acid sequence of SEQ ID NO: 398. Also described herein are polynucleotides that encode a TCR comprising an alpha chain and a beta chain, wherein the alpha chain comprises a CDR1 that is encoded by the nucleic acid sequence of SEQ ID NO: 156, a CDR2 that is encoded by the nucleic acid sequence of SEQ ID NO: 158, and a CDR3 that is encoded by the nucleic acid sequence of SEQ ID NO: 160, and the beta chain comprises a CDR1 that is encoded by the nucleic acid sequence of SEQ ID NO: 394, a CDR2 that is encoded by the nucleic acid sequence of SEQ ID NO: 396, and a CDR3 that is encoded by the nucleic acid sequence of SEQ ID NO: 398. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 544, and the beta chain comprises a VJD region that is encoded by a nucleic acid sequence that is at least 90%, 95%, 96%, 97%, 98%, 99% or 100% identical to the nucleic acid sequence of SEQ ID NO: 646. In certain embodiments, the alpha chain is encoded by a nucleic acid sequence that is at least 90%, 95%, 96%, 97%, 98%, 99% or 100% identical to the nucleic acid sequence of SEQ ID NO: 155, and the beta chain is encoded by a nucleic acid sequence that is at least 90%, 95%, 96%, 97%, 98%, 99% or 100% identical to the nucleic acid sequence of SEQ ID NO: 393.

Described herein are polynucleotides that encode a TCR comprising an alpha chain and a beta chain, wherein the alpha chain comprises a CDR3 that is encoded by the nucleic acid sequence of SEQ ID NO: 167, and the beta chain comprises a CDR3 that is encoded by the nucleic acid sequence of SEQ ID NO: 405. Also described herein are polynucleotides that encode a TCR comprising an alpha chain and a beta chain, wherein the alpha chain comprises a CDR1 that is encoded by the nucleic acid sequence of SEQ ID NO: 163, a CDR2 that is encoded by the nucleic acid sequence of SEQ ID NO: 165, and a CDR3 that is encoded by the nucleic acid sequence of SEQ ID NO: 167, and the beta chain comprises a CDR1 that is encoded by the nucleic acid sequence of SEQ ID NO: 401, a CDR2 that is encoded by the nucleic acid sequence of SEQ ID NO: 403, and a CDR3 that is encoded by the nucleic acid sequence of SEQ ID NO: 405. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 547, and the beta chain comprises a VJD region that is encoded by a nucleic acid sequence that is at least 90%, 95%, 96%, 97%, 98%, 99% or 100% identical to the nucleic acid sequence of SEQ ID NO: 649. In certain embodiments, the alpha chain is encoded by a nucleic acid sequence that is at least 90%, 95%, 96%, 97%, 98%, 99% or 100% identical to the nucleic acid sequence of SEQ ID NO: 162, and the beta chain is encoded by a nucleic acid sequence that is at least 90%, 95%, 96%, 97%, 98%, 99% or 100% identical to the nucleic acid sequence of SEQ ID NO: 400.

Described herein are polynucleotides that encode a TCR comprising an alpha chain and a beta chain, wherein the alpha chain comprises a CDR3 that is encoded by the nucleic acid sequence of SEQ ID NO: 174, and the beta chain comprises a CDR3 that is encoded by the nucleic acid sequence of SEQ ID NO: 412. Also described herein are polynucleotides that encode a TCR comprising an alpha chain and a beta chain, wherein the alpha chain comprises a CDR1 that is encoded by the nucleic acid sequence of SEQ ID NO: 170, a CDR2 that is encoded by the nucleic acid sequence of SEQ ID NO: 172, and a CDR3 that is encoded by the nucleic acid sequence of SEQ ID NO: 174, and the beta chain comprises a CDR1 that is encoded by the nucleic acid sequence of SEQ ID NO: 408, a CDR2 that is encoded by the nucleic acid sequence of SEQ ID NO: 410, and a CDR3 that is encoded by the nucleic acid sequence of SEQ ID NO: 412. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 550, and the beta chain comprises a VJD region that is encoded by a nucleic acid sequence that is at least 90%, 95%, 96%, 97%, 98%, 99% or 100% identical to the nucleic acid sequence of SEQ ID NO: 652. In certain embodiments, the alpha chain is encoded by a nucleic acid sequence that is at least 90%, 95%, 96%, 97%, 98%, 99% or 100% identical to the nucleic acid sequence of SEQ ID NO: 169, and the beta chain is encoded by a nucleic acid sequence that is at least 90%, 95%, 96%, 97%, 98%, 99% or 100% identical to the nucleic acid sequence of SEQ ID NO: 407.

Described herein are polynucleotides that encode a TCR comprising an alpha chain and a beta chain, wherein, the alpha chain comprises a CDR3 that is encoded by the nucleic acid sequence of SEQ ID NO: 181, and the beta chain comprises a CDR3 that is encoded by the nucleic acid sequence of SEQ ID NO: 419. Also described herein are polynucleotides that encode a TCR comprising an alpha chain and a beta chain, wherein the alpha chain comprises a CDR1 that is encoded by the nucleic acid sequence of SEQ ID NO: 177, a CDR2 that is encoded by the nucleic acid sequence of SEQ ID NO: 179, and a CDR3 that is encoded by the nucleic acid sequence of SEQ ID NO: 181, and the beta chain comprises a CDR1 that is encoded by the nucleic acid sequence of SEQ ID NO: 415, a CDR2 that is encoded by the nucleic acid sequence of SEQ ID NO: 417, and a CDR3 that is encoded by the nucleic acid sequence of SEQ ID NO: 419. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 553, and the beta chain comprises a VJD region that is encoded by a nucleic acid sequence that is at least 90%, 95%, 96%, 97%, 98%, 99% or 100% identical to the nucleic acid sequence of SEQ ID NO: 655. In certain embodiments, the alpha chain is encoded by a nucleic acid sequence that is at least 90%, 95%, 96%, 97%, 98%, 99% or 100% identical to the nucleic acid sequence of SEQ ID NO: 176, and the beta chain is encoded by a nucleic acid sequence that is at least 90%, 95%, 96%, 97%, 98%, 99% or 100% identical to the nucleic acid sequence of SEQ ID NO: 414.

Described herein are polynucleotides that encode a TCR comprising an alpha chain and a beta chain, wherein the alpha chain comprises a CDR3 that is encoded by the nucleic acid sequence of SEQ ID NO: 188, and the beta chain comprises a CDR3 that is encoded by the nucleic acid sequence of SEQ ID NO: 426. Also described herein are polynucleotides that encode a TCR comprising an alpha chain and a beta chain, wherein the alpha chain comprises a CDR1 that is encoded by the nucleic acid sequence of SEQ ID NO: 184, a CDR2 that is encoded by the nucleic acid sequence of SEQ ID NO: 186, and a CDR3 that is encoded by the nucleic acid sequence of SEQ ID NO: 188, and the beta chain comprises a CDR1 that is encoded by the nucleic acid sequence of SEQ ID NO: 422, a CDR2 that is encoded by the nucleic acid sequence of SEQ ID NO: 424, and a CDR3 that is encoded by the nucleic acid sequence of SEQ ID NO: 426. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 556, and the beta chain comprises a VJD region that is encoded by a nucleic acid sequence that is at least 90%, 95%, 96%, 97%, 98%, 99% or 100% identical to the nucleic acid sequence of SEQ ID NO: 658. In certain embodiments, the alpha chain is encoded by a nucleic acid sequence that is at least 90%, 95%, 96%, 97%, 98%, 99% or 100% identical to the nucleic acid sequence of SEQ ID NO: 183, and the beta chain is encoded by a nucleic acid sequence that is at least 90%, 95%, 96%, 97%, 98%, 99% or 100% identical to the nucleic acid sequence of SEQ ID NO: 421.

Described herein are polynucleotides that encode a TCR comprising an alpha chain and a beta chain, wherein the alpha chain comprises a CDR3 that is encoded by the nucleic acid sequence of SEQ ID NO: 195, and the beta chain comprises a CDR3 that is encoded by the nucleic acid sequence of SEQ ID NO: 433. Also described herein are polynucleotides that encode a TCR comprising an alpha chain and a beta chain, wherein the alpha chain comprises a CDR1 that is encoded by the nucleic acid sequence of SEQ ID NO: 191, a CDR2 that is encoded by the nucleic acid sequence of SEQ ID NO: 193, and a CDR3 that is encoded by the nucleic acid sequence of SEQ ID NO: 195, and the beta chain comprises a CDR1 that is encoded by the nucleic acid sequence of SEQ ID NO: 429, a CDR2 that is encoded by the nucleic acid sequence of SEQ ID NO: 431, and a CDR3 that is encoded by the nucleic acid sequence of SEQ ID NO: 433. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 559, and the beta chain comprises a VJD region that is encoded by a nucleic acid sequence that is at least 90%, 95%, 96%, 97%, 98%, 99% or 100% identical to the nucleic acid sequence of SEQ ID NO: 661. In certain embodiments, the alpha chain is encoded by a nucleic acid sequence that is at least 90%, 95%, 96%, 97%, 98%, 99% or 100% identical to the nucleic acid sequence of SEQ ID NO: 190, and the beta chain is encoded by a nucleic acid sequence that is at least 90%, 95%, 96%, 97%, 98%, 99% or 100% identical to the nucleic acid sequence of SEQ ID NO: 428.

Described herein are polynucleotides that encode a TCR comprising an alpha chain and a beta chain, wherein the alpha chain comprises a CDR3 that is encoded by the nucleic acid sequence of SEQ ID NO: 202, and the beta chain comprises a CDR3 that is encoded by the nucleic acid sequence of SEQ ID NO: 440. Also described herein are polynucleotides that encode a TCR comprising an alpha chain and a beta chain, wherein the alpha chain comprises a CDR1 that is encoded by the nucleic acid sequence of SEQ ID NO: 198, a CDR2 that is encoded by the nucleic acid sequence of SEQ ID NO: 200, and a CDR3 that is encoded by the nucleic acid sequence of SEQ ID NO: 202, and the beta chain comprises a CDR1 that is encoded by the nucleic acid sequence of SEQ ID NO: 436, a CDR2 that is encoded by the nucleic acid sequence of SEQ ID NO: 438, and a CDR3 that is encoded by the nucleic acid sequence of SEQ ID NO: 440. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 562, and the beta chain comprises a VJD region that is encoded by a nucleic acid sequence that is at least 90%, 95%, 96%, 97%, 98%, 99% or 100% identical to the nucleic acid sequence of SEQ ID NO: 664. In certain embodiments, the alpha chain is encoded by a nucleic acid sequence that is at least 90%, 95%, 96%, 97%, 98%, 99% or 100% identical to the nucleic acid sequence of SEQ ID NO: 197, and the beta chain is encoded by a nucleic acid sequence that is at least 90%, 95%, 96%, 97%, 98%, 99% or 100% identical to the nucleic acid sequence of SEQ ID NO: 435.

Described herein are polynucleotides that encode a TCR comprising an alpha chain and a beta chain, wherein the alpha chain comprises a CDR3 that is encoded by the nucleic acid sequence of SEQ ID NO: 209, and the beta chain comprises a CDR3 that is encoded by the nucleic acid sequence of SEQ ID NO: 447. Also described herein are polynucleotides that encode a TCR comprising an alpha chain and a beta chain, wherein the alpha chain comprises a CDR1 that is encoded by the nucleic acid sequence of SEQ ID NO: 205, a CDR2 that is encoded by the nucleic acid sequence of SEQ ID NO: 207, and a CDR3 that is encoded by the nucleic acid sequence of SEQ ID NO: 209, and the beta chain comprises a CDR1 that is encoded by the nucleic acid sequence of SEQ ID NO: 443, a CDR2 that is encoded by the nucleic acid sequence of SEQ ID NO: 445, and a CDR3 that is encoded by the nucleic acid sequence of SEQ ID NO: 447. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 565, and the beta chain comprises a VJD region that is encoded by a nucleic acid sequence that is at least 90%, 95%, 96%, 97%, 98%, 99% or 100% identical to the nucleic acid sequence of SEQ ID NO: 667. In certain embodiments, the alpha chain is encoded by a nucleic acid sequence that is at least 90%, 95%, 96%, 97%, 98%, 99% or 100% identical to the nucleic acid sequence of SEQ ID NO: 204, and the beta chain is encoded by a nucleic acid sequence that is at least 90%, 95%, 96%, 97%, 98%, 99% or 100% identical to the nucleic acid sequence of SEQ ID NO: 442.

Described herein are polynucleotides that encode a TCR comprising an alpha chain and a beta chain, wherein the alpha chain comprises a CDR3 that is encoded by the nucleic acid sequence of SEQ ID NO: 216, and the beta chain comprises a CDR3 that is encoded by the nucleic acid sequence of SEQ ID NO: 454. Also described herein are polynucleotides that encode a TCR comprising an alpha chain and a beta chain, wherein the alpha chain comprises a CDR1 that is encoded by the nucleic acid sequence of SEQ ID NO: 212, a CDR2 that is encoded by the nucleic acid sequence of SEQ ID NO: 214, and a CDR3 that is encoded by the nucleic acid sequence of SEQ ID NO: 216, and the beta chain comprises a CDR1 that is encoded by the nucleic acid sequence of SEQ ID NO: 450, a CDR2 that is encoded by the nucleic acid sequence of SEQ ID NO: 452, and a CDR3 that is encoded by the nucleic acid sequence of SEQ ID NO: 454. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 568, and the beta chain comprises a VJD region that is encoded by a nucleic acid sequence that is at least 90%, 95%, 96%, 97%, 98%, 99% or 100% identical to the nucleic acid sequence of SEQ ID NO: 670. In certain embodiments, the alpha chain is encoded by a nucleic acid sequence that is at least 90%, 95%, 96%, 97%, 98%, 99% or 100% identical to the nucleic acid sequence of SEQ ID NO: 211, and the beta chain is encoded by a nucleic acid sequence that is at least 90%, 95%, 96%, 97%, 98%, 99% or 100% identical to the nucleic acid sequence of SEQ ID NO: 449.

Described herein are polynucleotides that encode a TCR comprising an alpha chain and a beta chain, wherein the alpha chain comprises a CDR3 that is encoded by the nucleic acid sequence of SEQ ID NO: 223, and the beta chain comprises a CDR3 that is encoded by the nucleic acid sequence of SEQ ID NO: 461. Also described herein are polynucleotides that encode a TCR comprising an alpha chain and a beta chain, wherein the alpha chain comprises a CDR1 that is encoded by the nucleic acid sequence of SEQ ID NO: 219, a CDR2 that is encoded by the nucleic acid sequence of SEQ ID NO: 221, and a CDR3 that is encoded by the nucleic acid sequence of SEQ ID NO: 223, and the beta chain comprises a CDR1 that is encoded by the nucleic acid sequence of SEQ ID NO: 457, a CDR2 that is encoded by the nucleic acid sequence of SEQ ID NO: 459, and a CDR3 that is encoded by the nucleic acid sequence of SEQ ID NO: 461. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 571, and the beta chain comprises a VJD region that is encoded by a nucleic acid sequence that is at least 90%, 95%, 96%, 97%, 98%, 99% or 100% identical to the nucleic acid sequence of SEQ ID NO: 673. In certain embodiments, the alpha chain is encoded by a nucleic acid sequence that is at least 90%, 95%, 96%, 97%, 98%, 99% or 100% identical to the nucleic acid sequence of SEQ ID NO: 218, and the beta chain is encoded by a nucleic acid sequence that is at least 90%, 95%, 96%, 97%, 98%, 99% or 100% identical to the nucleic acid sequence of SEQ ID NO: 456.

Described herein are polynucleotides that encode a TCR comprising an alpha chain and a beta chain, wherein the alpha chain comprises a CDR3 that is encoded by the nucleic acid sequence of SEQ ID NO: 230, and the beta chain comprises a CDR3 that is encoded by the nucleic acid sequence of SEQ ID NO: 468. Also described herein are polynucleotides that encode a TCR comprising an alpha chain and a beta chain, wherein the alpha chain comprises a CDR1 that is encoded by the nucleic acid sequence of SEQ ID NO: 226, a CDR2 that is encoded by the nucleic acid sequence of SEQ ID NO: 228, and a CDR3 that is encoded by the nucleic acid sequence of SEQ ID NO: 230, and the beta chain comprises a CDR1 that is encoded by the nucleic acid sequence of SEQ ID NO: 464, a CDR2 that is encoded by the nucleic acid sequence of SEQ ID NO: 466, and a CDR3 that is encoded by the nucleic acid sequence of SEQ ID NO: 468. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 574, and the beta chain comprises a VJD region that is encoded by a nucleic acid sequence that is at least 90%, 95%, 96%, 97%, 98%, 99% or 100% identical to the nucleic acid sequence of SEQ ID NO: 676. In certain embodiments, the alpha chain is encoded by a nucleic acid sequence that is at least 90%, 95%, 96%, 97%, 98%, 99% or 100% identical to the nucleic acid sequence of SEQ ID NO: 225, and the beta chain is encoded by a nucleic acid sequence that is at least 90%, 95%, 96%, 97%, 98%, 99% or 100% identical to the nucleic acid sequence of SEQ ID NO: 463.

Described herein are polynucleotides that encode a TCR comprising an alpha chain and a beta chain, wherein the alpha chain comprises a CDR3 that is encoded by the nucleic acid sequence of SEQ ID NO: 237, and the beta chain comprises a CDR3 that is encoded by the nucleic acid sequence of SEQ ID NO: 475. Also described herein are polynucleotides that encode a TCR comprising an alpha chain and a beta chain, wherein the alpha chain comprises a CDR1 that is encoded by the nucleic acid sequence of SEQ ID NO: 233, a CDR2 that is encoded by the nucleic acid sequence of SEQ ID NO: 235, and a CDR3 that is encoded by the nucleic acid sequence of SEQ ID NO: 237, and the beta chain comprises a CDR1 that is encoded by the nucleic acid sequence of SEQ ID NO: 471, a CDR2 that is encoded by the nucleic acid sequence of SEQ ID NO: 473, and a CDR3 that is encoded by the nucleic acid sequence of SEQ ID NO: 475. In certain embodiments, the alpha chain comprises a VJ region that is encoded by a nucleic acid sequence that is at least 90%, 95%, 96%, 97%, 98%, 99% or 100% identical to the nucleic acid sequence of SEQ ID NO: 577, and the beta chain comprises a VJD region that is encoded by a nucleic acid sequence that is at least 90%, 95%, 96%, 97%, 98%, 99% or 100% identical to the nucleic acid sequence of SEQ ID NO: 679. In certain embodiments, the alpha chain is encoded by a nucleic acid sequence that is at least 90%, 95%, 96%, 97%, 98%, 99% or 100% identical to the nucleic acid sequence of SEQ ID NO: 232, and the beta chain is encoded by a nucleic acid sequence that is at least 90%, 95%, 96%, 97%, 98%, 99% or 100% identical to the nucleic acid sequence of SEQ ID NO: 470.

In some embodiments, the polynucleotide comprises DNA.

In some embodiments, the polynucleotide comprises RNA.

In some embodiments, the RNA is mRNA.

In some embodiments, the polynucleotide comprises a promoter, an enhancer, a polyadenylation site, a Kozak sequence, a stop codon, or any combination thereof.

Methods of generating polynucleotides of the disclosure are known in the art and include chemical synthesis, enzymatic synthesis (e.g. in vitro transcription), enzymatic or chemical cleavage of a longer precursor, chemical synthesis of smaller fragments of the polynucleotides followed by ligation of the fragments or known PCR methods. The polynucleotide sequence to be synthesized may be designed with the appropriate codons for the desired amino acid sequence. In general, preferred codons may be selected for the intended host in which the sequence will be used for expression.

### Vectors

The disclosure also provides vectors comprising any of the polynucleotides disclosed herein. The disclosure also provides vectors comprising a polynucleotide encoding for any of the polypeptides disclosed herein.

Described herein are vectors comprising polynucleotides that encode a TCR comprising an alpha chain and a beta chain, wherein the alpha chain comprises a CDR3 comprising an amino acid sequence provided in Table 4, and the corresponding beta chain comprises a CDR3 comprising a corresponding amino acid sequence provided in Table 5. Described herein are vectors comprising polynucleotides that encode a TCR comprising an alpha chain and a beta chain, wherein the alpha chain comprises a CDR3 that is encoded by a nucleic acid sequence provided in Table 4, and the corresponding beta chain comprises a CDR3 that is encoded by a corresponding nucleic acid sequence provided in Table 5.

Also described herein are vectors comprising polynucleotides that encode a TCR comprising an alpha chain and a beta chain, wherein the alpha chain comprises a CDR1 comprising the amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5.

Also described herein are vectors comprising polynucleotides that encode a TCR comprising an alpha chain and a beta chain, wherein the alpha chain comprises a CDR1 that is encoded by a nucleic acid sequence provided in Table 4, a CDR2 that is encoded by a nucleic acid sequence provided in Table 4, and a CDR3 that is encoded by a nucleic acid sequence provided in Table 4, and the corresponding beta chain comprises a CDR1 that is encoded by a nucleic acid sequence provided in Table 5, a CDR2 that is encoded by a nucleic acid sequence provided in Table 5, and a CDR3 that is encoded by a nucleic acid sequence provided in Table 5.

Also described herein are vectors comprising polynucleotides that encode a TCR comprising an alpha chain and a beta chain, wherein the alpha chain comprises a VJ region comprising an amino acid sequence that is at least 90%, 95%, 96%, 97%, 98%, 99% or 100% identical to an amino acid sequence provided in Table 4, and the corresponding beta chain comprises a VDJ region comprising an amino acid sequence that is at least 90%, 95%, 96%, 97%, 98%, 99% or 100% identical to an amino acid sequence provided in Table 5.

Also described herein are vectors comprising polynucleotides that encode a TCR comprising an alpha chain and a beta chain, wherein the alpha chain comprises a VJ region that is encoded by a nucleic acid sequence that is at least 90%, 95%, 96%, 97%, 98%, 99% or 100% identical to a nucleic acid sequence provided in Table 4, and the corresponding beta chain comprises a VDJ region that is encoded by a nucleic acid sequence that is at least 90%, 95%, 96%, 97%, 98%, 99% or 100% identical to a nucleic acid sequence provided in Table 5.

Also described herein are vectors comprising polynucleotides that encode a TCR comprising an alpha chain and a beta chain, wherein the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5.

Also described herein are vectors comprising polynucleotides that encode a TCR polypeptide comprising an alpha chain and a beta chain, wherein the alpha chain is encoded by a nucleic acid sequence that is at least 90%, 95%, 96%, 97%, 98%, 99% or 100% identical to a nucleic acid sequence provided in Table 4, and the corresponding beta chain is encoded by a nucleic acid sequence that is at least 90%, 95%, 96%, 97%, 98%, 99% or 100% identical to a nucleic acid sequence provided in Table 5.

The vector may be a vector intended for expression of the polynucleotide of the disclosure in any host, such as bacteria, yeast, or a mammal. Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed or transduced with the desired DNA sequences. Exemplary vectors are plasmids, cosmids, phages, viral vectors, or artificial chromosomes.

Suitable vectors that may be used include, but are not limited to—Bacterial: pBs, phagescript, PsiX174, pBluescript SK, pBs KS, pNH8a, pNH16a, pNH18a, pNH46a (Stratagene, La Jolla, Calif., USA), pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene), pSVK3, pBPV, pMSG and pSVL (Pharmacia).

The disclosure provides an expression vector comprising the polynucleotide of the disclosure. The disclosure also provides an expression vector comprising the polynucleotide encoding for the polypeptide of the disclosure.

### Other Viral Vectors and Recombinant Viruses

The disclosure also provides a viral vector comprising any of the polynucleotides of the disclosure.

The disclosure also provides a viral vector comprising a polynucleotide encoding any of the TCR polypeptides of the disclosure.

Viral vectors are derived from naturally occurring virus genomes, which typically are modified to be replication incompetent, e.g. non-replicating. Non-replicating viruses require the provision of proteins in trans for replication. Typically, those proteins are stably or transiently expressed in a viral producer cell line, thereby allowing replication of the virus. The viral vectors are, thus, typically infectious, and non-replicating. Viral vectors may be adenovirus vectors, adeno-associated virus (AAV) vectors (e.g., AAV type 5 and type 2), Great ape adenovirus vectors (GAd), alphavirus vectors (e.g., Venezuelan equine encephalitis virus (VEEV), Sindbis virus (SIN), Semliki forest virus (SFV), and VEE-SIN chimeras), herpes virus vectors (e.g. vectors derived from cytomegaloviruses, like rhesus cytomegalovirus (RhCMV)), arena virus vectors (e.g. lymphocytic choriomeningitis virus (LCMV) vectors), measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), vesicular stomatitis virus vectors, retrovirus vectors, lentivirus vectors, viral like particles, LNP encapsulated self-replicating RNA derived from alpha virus, and bacterial spores.

In some embodiments, the viral vector is derived from adenovirus, poxvirus, alphavirus, adeno-associated virus, retrovirus, or a self-replicating RNA molecule.

In some embodiments, the viral vector is derived from an adenovirus. Suitable adenovirus vectors include, for example, hAd5, hAd7, hAd11, hAd26, hAd34, hAd35, hAd48, hAd49, hAd50, GAd20, Gad19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAdI7, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, and PanAd3.

Adenovirus vectors may be derived from human adenovirus (Ad) but also from adenoviruses that infect other species, such as bovine adenovirus (e.g. bovine adenovirus 3, BAdV3), a canine adenovirus (e.g. CAdV2), a porcine adenovirus (e.g. PAdV3 or 5), or great apes, such as Chimpanzee (Pan), Gorilla (Gorilla), Orangutan (Pongo), Bonobo (Pan paniscus) and common chimpanzee (Pan troglodytes). Typically, naturally occurring great ape adenoviruses are isolated from stool samples of the respective great ape.

Human adenovirus vectors may be derived from various adenovirus serotypes, for example from human adenovirus serotypes hAd5, hAd7, hAd11, hAd26, hAd34, hAd35, hAd48, hAd49 or hAd50 (the serotypes are also referred to as Ad5, Ad7, Ad11, Ad26, Ad34, Ad35, Ad48, Ad49 or Ad50).

Great ape adenovirus (GAd) vectors may be derived from various adenovirus serotypes, for example from great ape adenovirus serotypes GAd20, GAd19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAdI7, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, or PanAd3.

Adenovirus vectors are known in the art. The sequences of most of the human and non-human adenoviruses are known, and for others can be obtained using routine procedures. An exemplary genome sequence of Ad26 is found in GenBank Accession number EF153474 and in Int. Pat. Publ. No. WO2007/104792. An exemplary genome sequence of Ad35 is found in Int. Pat. Publ. No. WO2000/70071. Vectors based on Ad26 are described for example, in Int. Pat. Publ. No. WO2007/104792. Vectors based on Ad35 are described for example in U.S. Pat. No. 7,270,811 and Int. Pat. Publ. No. WO2000/70071. Vectors based on ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63 and ChAd82 are described in WO2005/071093. Vectors based on PanAd1, PanAd2, PanAd3, ChAd55, ChAd73, ChAd83, ChAd146, and ChAd147 are described in Int. Pat. Publ. No. WO2010/086189.

In some embodiments, the viral vector is a poxvirus. In some embodiments, the poxvirus vector is selected from smallpox virus vector, vaccinia virus vector, cowpox virus vector, monkeypox virus vector, Copenhagen vaccinia virus (W) vector, New York Attenuated Vaccinia Virus (NYVAC) vector, and Modified Vaccinia Ankara (MVA) vector.

Poxvirus (Poxviridae) vectors may be derived from smallpox virus (variola), vaccinia virus, cowpox virus or monkeypox virus. Exemplary vaccinia viruses are the Copenhagen vaccinia virus (W), New York Attenuated Vaccinia Virus (NYVAC), ALVAC, TROVAC or Modified Vaccinia Ankara (MVA).

MVA originates from the dermal vaccinia strain Ankara (Chorioallantois vaccinia Ankara (CVA) virus) that was maintained in the Vaccination Institute, Ankara, Turkey for many years and used as the basis for vaccination of humans.

In some embodiments, the viral vector is an adeno-associated virus. The viral vector comprising the polynucleotides of the disclosure may be derived from human adeno-associated viruses, such as AAV-2 (adeno-associated virus type 2). An attractive feature of AAV vectors is that they do not express any viral genes. The only viral DNA sequences included in the AAV vectors are the 145 bp inverted terminal repeats (ITR). Thus, as in immunization with naked DNA, the only gene expressed is that of the antigen, or antigen chimera. Additionally, AAV vectors are known to transduce both dividing and non-dividing cells, such as human peripheral blood monocyte-derived dendritic cells, with persistent transgene expression, and with the possibility of oral and intranasal delivery for generation of mucosal immunity. Moreover, the amount of DNA required appears to be much less by several orders of magnitude, with maximum responses at doses of 10¹⁰ particles or copies of DNA in contrast to naked DNA doses of 50 μg or about 10¹⁵ copies. AAV vectors are packaged by co-transfection of a suitable cell line (e.g., human 293 cells) with the DNA contained in the AAV ITR chimeric protein encoding constructs and an AAV helper plasmid ACG2 containing the AAV coding region (AAV rep and cap genes) without the ITRs. The cells are subsequently infected with the adenovirus Ad5. Vectors can be purified from cell lysates using methods known in the art (e.g., such as cesium chloride density gradient ultracentrifugation) and are validated to ensure that they are free of detectable replication-competent AAV or adenovirus (e.g., by a cytopathic effect bioassay).

The viral vector comprising the polynucleotide of the disclosure also include Retroviral vectors. Retroviruses are a class of integrative viruses which replicate using a virus-encoded reverse transcriptase, to replicate the viral RNA genome into double stranded DNA which is integrated into chromosomal DNA of the infected cells (e.g., target cells). Such vectors include those derived from murine leukemia viruses, especially Moloney (Gilboa, et al., 1988, Adv. Exp. Med. Biol. 241: 29) or Friend's FB29 strains (Int. Pat. Publ. No. WO1995/01447). Generally, a retroviral vector contains deletions of all or part of the viral genes gag, pol and env and retains 5′ and 3′ LTRs and an encapsidation sequence. These elements may be modified to increase expression level or stability of the retroviral vector. Such modifications include the replacement of the retroviral encapsidation sequence by a retrotransposon such as VL30 (see, e.g., U.S. Pat. No. 5,747,323).

The polynucleotides encoding the polypeptide of the disclosure may be inserted downstream of the encapsidation sequence, such as in opposite direction relative to the retroviral genome. Retroviral particles are prepared in the presence of a helper virus or in an appropriate complementation (packaging) cell line which contains integrated into its genome the retroviral genes for which the retroviral vector is defective (e.g. gag/pol and env). Such cell lines are described in Miller and Rosman, 1989, BioTechniques 7: 980; Danos and Mulligan, 1988, Proc. Natl. Acad. Sci. USA 85: 6460; Markowitz, et al., 1988, Virol. 167: 400. The product of the env gene is responsible for the binding of the viral particle to the viral receptors present on the surface of the target cell and, therefore determines the host range of the retroviral particle. Packaging cell line, such as the PA317 cells (ATCC CRL 9078) or 293EI6 (WO97/35996) containing an amphotropic envelope protein may therefore be used to allow infection of human and other species' target cells. The retroviral particles are recovered from the culture supernatant and may optionally be further purified according to standard techniques (e.g. chromatography, ultracentrifugation).

The disclosure also provides a host cell comprising any of the above vectors.

Suitable host cells include prokaryotic or eukaryotic host cells. In some embodiments, the host cell is PER.C6, PER.C6 TetO, a chicken embryo fibroblast (CEF), CHO, HEK293, HT-1080, HKB-11, CAP, HuH-7, or Agel cell line.

### Self-Replicating RNA Molecules

Provided herein is a viral vector comprising any of the polynucleotides of the disclosure, wherein the vector is a self-replicating RNA molecule.

Self-replicating RNA may be derived from alphavirus. Alphaviruses may belong to the VEEV/EEEV group, or the SF group, or the SIN group. Non-limiting examples of SF group alphaviruses include Semliki Forest virus, O'Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S. A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

The self-replicating RNA molecules can be derived from alphavirus genomes, meaning that they have some of the structural characteristics of alphavirus genomes, or similar to them. The self-replicating RNA molecules can be derived from modified alphavirus genomes.

Self-replicating RNA molecules may be derived from Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. Virulent and avirulent alphavirus strains are both suitable. In some embodiments, the alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV).

In some embodiments, the alphavirus-derived self-replicating RNA molecule is a Venezuelan equine encephalitis virus (VEEV).

The self-replicating RNA molecules can contain RNA sequences from (or amino acid sequences encoded by) a wild-type New World or Old World alphavirus genome. Any of the self-replicating RNA molecules disclosed herein can contain RNA sequences “derived from” or “based on” wild type alphavirus genome sequences, meaning that they have at least 60% or at least 65% or at least 68% or at least 70% or at least 80% or at least 85% or at least 90% or at least 95% or at least 97% or at least 98% or at least 99% or 100% or 80-99% or 90-100% or 95-99% or 95-100% or 97-99% or 98-99% sequence identity with an RNA sequence (which can be a corresponding RNA sequence) from a wild type RNA alphavirus genome, which can be a New World or Old World alphavirus genome.

Self-replicating RNA molecules contain all of the genetic information required for directing their own amplification or self-replication within a permissive cell. To direct their own replication, self-replicating RNA molecules encode polymerase, replicase, or other proteins which may interact with viral or host cell-derived proteins, nucleic acids or ribonucleoproteins to catalyze the RNA amplification process; and contain cis-acting RNA sequences required for replication and transcription of the replicon-encoded RNA. Thus, RNA replication leads to the production of multiple daughter RNAs. These daughter RNAs, as well as collinear subgenomic transcripts, can be translated to provide in situ expression of a gene of interest, or can be transcribed to provide further transcripts with the same sense as the delivered RNA which are translated to provide in situ expression of the gene of interest. The overall results of this sequence of transcriptions is a huge amplification in the number of the introduced replicon RNAs and so the encoded gene of interest becomes a major polypeptide product of the cells.

There are two open reading frames (ORF's) in the genome of alphaviruses, non-structural (ns) and structural genes. The ns ORF encodes proteins (nsP1-nsP4) necessary for transcription and replication of viral RNA and are produced as a polyprotein and are the virus replication machinery. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome.

Self-replicating RNA molecules can be used as basis of introducing foreign sequences to host cells by replacing viral sequences encoding structural genes or inserting the foreign sequences 5′ or 3′ of the sequences encoding the structural genes. They can be engineered to replace the viral structural genes downstream of the replicase, which are under control of a subgenomic promoter, by genes of interest (GOI), e.g. the polynucleotide encoding for the polypeptide of the disclosure. Upon transfection, the replicase which is translated immediately, interacts with the 5′ and 3′ termini of the genomic RNA, and synthesizes complementary genomic RNA copies. Those act as templates for the synthesis of novel positive-stranded, capped, and poly-adenylated genomic copies, and subgenomic transcripts. Amplification eventually leads to very high RNA copy numbers of up to 2×10⁵ copies per cell. The result is a uniform and/or enhanced expression of a GOI (e.g. the polynucleotide encoding for the polypeptide of the disclosure) that can affect efficacy or therapeutic impact of a treatment.

The disclosure provides a self-replicating RNA molecule containing all of the genetic information required for directing its own amplification or self-replication within a permissive cell.

The disclosure also provides a self-replicating RNA molecule that can be used as the basis of introducing foreign sequences to host cells (e.g. the polynucleotides encoding the polypeptides of the disclosure) by replacing viral sequences encoding structural genes.

Provided herein is a viral vector comprising any of the polynucleotides of the disclosure, wherein the vector is a self-replicating RNA molecule.

Any of the above self-replicating RNA molecules can further comprise one or more of the following:
one or more nonstructural genes nsP1, nsP2, nsP3 and nsP4;
at least one of a DLP motif, a 5′ UTR, a 3′UTR and a Poly A; and
a subgenomic promoter.

In some embodiments, for example, the self-replicating RNA molecule can comprise one or more of the following:
one or more nonstructural genes nsP1, nsP2, nsP3 and nsP4;
at least one of a DLP motif, a 5′ UTR, a 3′UTR and a Poly A; and
a subgenomic promoter; and
an RNA encoding for amino acids of SEQ ID NOs: 8 or 10, and operably linked to the subgenomic promoter.

In some embodiments, the self-replicating RNA molecule comprises an RNA sequence encoding a protein or peptide; 5′ and 3′ alphavirus untranslated regions; RNA sequences encoding amino acid sequences derived from New World alphavirus VEEV nonstructural proteins nsP1, nsP2, nsP3 and nsP4; a sub-genomic promoter that is operably linked to and regulates translation of the RNA sequence encoding the protein; a 5′ cap and a 3′ poly-A tail; positive sense, single-stranded RNA; a DLP from Sindbis virus upstream of the non-structural protein 1 (nsP1); a 2A ribosome skipping element; and a nspl nucleotide repeat downstream of the 5′-UTR and upstream of the DLP.

In some embodiments, the self-replicating RNA molecules may be at least 1 kb or at least 2 kb or at least 3 kb or at least 4 kb or at least 5 kb or at least 6 kb or at least 7 kb or at least 8 kb or at least 10 kb or at least 12 kb or at least 15 kb or at least 17 kb or at least 19 kb or at least 20 kb in size, or can be 100 bp-8 kb or 500 bp-8 kb or 500 bp-7 kb or 1-7 kb or 1-8 kb or 2-15 kb or 2-20 kb or 5-15 kb or 5-20 kb or 7-15 kb or 7-18 kb or 7-20 kb in size.

Any of the above-disclosed self-replicating RNA molecules can further include a coding sequence for an autoprotease peptide (e.g., autocatalytic self-cleaving peptide), where the coding sequence for the autoprotease is optionally operably linked upstream to the nucleic acid sequence encoding the GOI.

Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoprotease” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteases have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro and in vivo eukaryotic systems. As such, the concept of autoproteases is available to one of skill in the art as many naturally occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoprotease peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequence for the autoprotease peptide is operably linked downstream of the DLP motif and upstream to the GOI.

In some embodiments, the autoprotease peptide comprises, or consists of, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence of porcine teschovirus-1 2A (P2A).

In some embodiments, the autoprotease peptide is selected from the group consisting of porcine teschovirus-1 2A (P2A), foot-and-mouth disease virus (FMDV) 2A (F2A), Equine Rhinitis A Virus (ERAV) 2A (E2A), Thosea asigna virus 2A (T2A), cytoplasmic polyhedrosis virus 2A (BmCPV2A), Flacherie Virus 2A (BmIFV2A), and a combination thereof.

In some embodiments, the autoprotease peptide is porcine teschovirus-1 2A (P2A).

The incorporation of the P2A peptide in the modified viral RNA replicons allows release of protein encoded by GOI (e.g. the polynucleotides encoding the TCR polypeptides of the disclosure) from the capsid-GOI fusion.

In some embodiments disclosed herein, the porcine teschovirus-1 2A (P2A) peptide sequence is engineered in-frame immediately after the DLP sequence and in-frame immediately upstream of all GOI.

Any of the above-disclosed self-replicating RNA molecules can further include a coding sequence downstream Loop (DLP) motif.

Some viruses have sequences capable of forming one or more stem-loop structures which regulate, for example increase, capsid gene expression. Viral capsid enhancer as used herein refers to a regulatory element comprising sequences capable of forming such stem-loop structures. In some examples, the stem-loop structures are formed by sequences within the coding sequence of a capsid protein and named Downstream Loop (DLP) sequence. As disclosed herein, these stem-loop structures or variants thereof can be used to regulate, for example increase, expression level of genes of interest. For example, these stem-loop structures or variants thereof can be used in a recombinant vector (e.g., in a heterologous viral genome) for enhancing transcription and/or translation of coding sequence operably linked downstream thereto.

Alphavirus replication in host cells is known to induce the double-stranded RNA-dependent protein kinase (PKR). PKR phosphorylates the eukaryotic translation initiation factor 2α (eIF2α). Phosphorylation of eIF2α blocks translation initiation of mRNA and in doing so keeps viruses from a completing a productive replication cycle. Infection of cells with Sindbis virus induces PKR that results in phosphorylation of eIF2α, yet the viral subgenomic mRNA is efficiently translated while translation of all other cellular mRNAs is restricted. The efficient translation of the viral subgenomic mRNA in Sindbis virus is made possible by the presence of a stable RNA hairpin loop (or DLP motif) located downstream of the wild type AUG initiator codon for the virus capsid protein (e.g., capsid enhancer). It has been reported that the DLP structure can stall a ribosome on the wild type AUG and this supports translation of the subgenomic mRNA without the requirement for functional eIF2α. Thus, subgenomic mRNAs of Sindbis virus (SINV) as well as of other alphaviruses are efficiently translated even in cells that have highly active PKR resulting in complete phosphorylation of eIF2α.

The DLP structure was first characterized in Sindbis virus (SINV) 26S mRNA and also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The predicted structures of these Alphavirus 26S mRNAs were constructed based on SHAPE (selective 2′-hydroxyl acylation and primer extension) data (Toribio et al., Nucleic Acids Res. May 19; 44(9):4368-80, 2016), the content of which is hereby incorporated by reference). Stable stem-loop structures were detected in all cases except for CHIKV and ONNV, whereas MAYV and EEEV showed DLPs of lower stability (Toribio et al., 2016 supra). The highest DLP activities were reported for those Alphaviruses that contained the most stable DLP structures.

As an example, members of the Alphavirus genus can resist the activation of antiviral RNA-activated protein kinase (PKR) by means of the downstream loop (DLP) present within viral 26S transcripts, which allows an eIF2-independent translation initiation of these mRNAs. The downstream loop (DLP), is located downstream from the AUG in SINV 26S mRNA and in other members of the Alphavirus genus.

In some embodiments, the nucleic acid molecules of the disclosure can include a coding sequence for a GOI operably linked to DLP motif(s) and/or the coding sequence for the DLP motifs.

In some embodiments, the self-replicating RNA molecule of the disclosure comprises a downstream loop (DLP).

In some embodiments, the downstream loop (DLP) comprises at least one RNA-stem-loop.

In some instances, DLP activity depends on the distance between the DLP motif and the initiation codon AUG (AUGi). The AUG-DLP spacing in Alphavirus 26S mRNAs is tuned to the topology of the ES6S region of the ribosomal 18S rRNA in a way that allows the placement of the AUGi in the P site of the 40S subunit stalled by the DLP, allowing the incorporation of Met-tRNA without the participation of eIF2. In the case of Sindbis virus, the DLP motif is found in the first ^{˜}150 nt of the Sindbis subgenomic RNA. The hairpin is located downstream of the Sindbis capsid AUG initiation codon (AUG at nt 50 of the Sindbis subgenomic RNA) and results in stalling a ribosome such that the correct capsid gene AUG is used to initiate translation. Previous studies of sequence comparisons and structural RNA analysis revealed the evolutionary conservation of DLP in SINV and predicted the existence of equivalent DLP structures in many members of the Alphavirus genus (see e.g., Ventoso, J. Virol. 9484-9494, Vol. 86, September 2012).

Without being bound by any particular theory, it is believed that placing the DLP motif upstream of a coding sequence for any GOI typically results in a fusion-protein of N-terminal capsid amino acids that are encoded in the hairpin region to the GOI encoded protein because initiation occurs on the capsid AUG not the GOI AUG.

In some embodiments, the self-replicating RNA molecule comprises a downstream loop placed upstream of the non-structural protein 1 (nsP1).

In some embodiments, the downstream loop is placed upstream of the non-structural protein 1 (nsP1) and is joined to the nsP1 by a porcine teschovirus-1 2A (P2A) ribosome skipping element.

The DLP-containing self-replicating RNA of the disclosure can be useful in conferring a resistance to the innate immune system in a subject. Unmodified RNA replicons are sensitive to the initial innate immune system state of cells they are introduced into. If the cells/individuals are in a highly active innate immune system state, the RNA replicon performance (e.g., replication and expression of a GOI) can be negatively impacted. By engineering a DLP to control initiation of protein translation, particularly of non-structural proteins, the impact of the pre-existing activation state of the innate immune system to influence efficient RNA replicon replication is removed or lessened. The result is more uniform and/or enhanced expression of a GOI that can impact efficacy or therapeutic impact of a treatment.

The DLP motif of the self-replicating RNA can confer efficient mRNA translation in cellular environments where cellular mRNA translation is inhibited. When a DLP is linked with translation of a replicon vector's non-structural protein genes the replicase and transcriptase proteins are capable of initiating functional replication in PKR activated cellular environments. When a DLP is linked with translation of subgenomic mRNAs robust GOI expression is possible even when cellular mRNA is restricted due to innate immune activation. Accordingly, engineering self-replicating RNA that contain DLP structures to help drive translation of both non-structural protein genes and subgenomic mRNAs provides a powerful way to overcome innate immune activation.

Examples of a self-replicating RNA vector comprising a DLP motif are described in US Patent Application Publication US2018/0171340 and the International Patent Application Publication WO2018106615, the content of which is incorporated herein by reference in its entirety.

Any of the above-disclosed self-replicating RNA molecules can further comprise nonstructural genes nsP1, nsP2, nsP3 and/or nsP4. In some embodiments, the self-replicating RNA molecule does not encode a functional viral structural protein.

Alphavirus genomes encode non-structural proteins nsP1, nsP2, nsP3, and nsP4, which are produced as a single polyprotein precursor, sometimes designated P1234 (or nsP1-4 or nsP1234), and which is cleaved into the mature proteins through proteolytic processing. nsP1 can be about 60 kDa in size and may have methyltransferase activity and be involved in the viral capping reaction. nsP2 has a size of about 90 kDa and may have helicase and protease activity while nsP3 is about 60 kDa and contains three domains: a macrodomain, a central (or alphavirus unique) domain, and a hypervariable domain (HVD). nsP4 is about 70 kDa in size and contains the core RNA-dependent RNA polymerase (RdRp) catalytic domain. After infection the alphavirus genomic RNA is translated to yield a P1234 polyprotein, which is cleaved into the individual proteins.

Alphavirus genomes also encode three structural proteins: the core nucleocapsid protein C, and the envelope proteins P62 and E1 that associate as a heterodimer. Structural proteins are under the control of a subgenomic promoter and can be replaced by the GIO.

In some embodiments, the self-replicating RNA can lack (or not contain) the sequence(s) of at least one (or all) of the structural viral proteins (e.g. nucleocapsid protein C, and envelope proteins P62, 6K, and E1). In these embodiments, the sequences encoding one or more structural genes can be substituted with one or more sequences such as, for example, a coding sequence for at least one protein or peptide (or other gene of interest (GOI)) e.g. the TCR polypeptides of the disclosure.

In some embodiments, the self-replicating RNA lack sequences encoding alphavirus structural proteins; or do not encode alphavirus (or, optionally, any other) structural proteins. In some embodiments, the self-replicating RNA molecules are further devoid of a part or the entire coding region for one or more viral structural proteins. For example, the alphavirus expression system may be devoid of a portion of or the entire coding sequence for one or more of the viral capsid protein C, E1 glycoprotein, E2 glycoprotein, E3 protein and 6K protein.

In some embodiments, the self-replicating RNA molecule does not contain coding sequences for at least one of the structural viral proteins. In these instances, the sequences encoding structural genes can be substituted with one or more sequences such as, for example, a coding sequence for a GOI (e.g., the TCR polypeptides).

The disclosure also provides a self-replicating RNA molecule comprising nonstructural genes nsP1, nsP2, nsP3 and nsP4, and wherein the self-replicating RNA molecule does not encode a functional viral structural protein.

In some embodiments, the disclosure provides a self-replicating RNA molecule comprising the coding sequence for at least one, at least two, at least three, or at least four nonstructural viral proteins (e.g. nsP1, nsP2, nsP3, nsP4). The nsP1, nsP2, nsP3, and nsP4 proteins encoded by the replicon are functional or biologically active proteins.

In some embodiments, the self-replicating RNA molecule includes the coding sequence for a portion of the at least one nonstructural viral protein. For example, the self-replicating RNA molecules can include about 10%, 20%, 30%, 40%, 50%, 60%, 70%, 80%, 90%, 95%, 100%, or a range between any two of these values, of the encoding sequence for the at least one nonstructural viral protein. In some embodiments, the self-replicating RNA molecule can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993).

In some embodiments, the self-replicating RNA molecule can include the entire coding sequence for the at least one nonstructural protein. In some embodiments, the self-replicating RNA molecule comprises substantially all the coding sequence for the native viral nonstructural proteins. In certain embodiments, the one or more nonstructural viral proteins are derived from the same virus.

In some embodiments, the downstream loop DLP of the self-replicating RNA molecule placed upstream of the non-structural protein 1 (nsP1) is derived from Sindbis virus.

In some embodiments, the self-replicating RNA molecule comprises nsP1, nsP2, nsP3, and nsP4 sequences derived from the Venezuelan equine encephalitis virus (VEEV) and a DLP motif derived from the Sindbis virus (SIN).

In some embodiments, the self-replicating RNA molecules also have an RNA sub-sequence encoding an amino acid sequence derived from an alphavirus nsP3 macro domain, and an RNA sub-sequence encoding an amino acid sequence derived from an alphavirus nsP3 central domain. The self-replicating RNA molecules can also have an RNA sub-sequence encoding an amino acid sequence derived entirely from an Old World alphavirus nsP3 hypervariable domain, or can have an amino acid sequence having a portion derived from a New World alphavirus nsP3 hypervariable domain and a portion derived from an Old World alphavirus nsP3 hypervariable domain. i.e. the hyper variable domain (HVD) can be a hybrid or chimeric New World/Old World sequence.

In some embodiments, the self-replicating RNA molecules can have an RNA sequence encoding amino acid sequences derived from wild type New World alphavirus nsP1, nsP2, nsP3, and nsP4 protein sequences. In other embodiments, the one or more nonstructural proteins are derived from different viruses.

In some embodiments, the self-replicating RNA molecule may have an RNA sequence encoding an nsP3 macro domain derived from a wild type alphavirus nsP3, and an nsP3 central domain derived from a wild type alphavirus nsP3. In various embodiments, the macro and central domain(s) can both be derived from a New World wild type alphavirus nsP3 or can both be derived from an Old World wild type alphavirus nsP3 protein. In other embodiments, the macro domain can be derived from a New World wild type alphavirus macro domain and the central domain can be derived from an Old World wild type alphavirus central domain, or vice versa. The various domains can be of any sequence described herein.

In some embodiments, the self-replicating RNA molecule contains non VEEV nonstructural proteins nsP1, nsP2, nsP3 and nsP4.

The accumulated experimental evidence has demonstrated that replication/amplification of VEEV and other alphavirus genomes and their defective interfering (DI) RNAs is determined by three promoter elements: (i) the conserved 3′-terminal sequence element (3′ CSE) and the following poly(A) tail; (ii) the 5′ UTR, which functions as a key promoter element for both negative- and positive-strand RNA synthesis; and (iii) the 51-nt conserved sequence element (51-nt CSE), which is located in the nsP1-coding sequence and functions as an enhancer of alphavirus genome replication (Kim et al., PNAS, 2014, 111: 10708-10713, and references therein).

Any of the above-disclosed self-replicating RNA molecules can further include an unmodified 5′ untranslated region (5′UTR).

Previous studies have demonstrated that during VEEV and Sindbis virus infections only a small portion of viral nonstructural proteins (nsPs) is colocalized with dsRNA replication intermediates. Thus, it appears that a large fraction of nsPs are not involved in RNA replication (Gorchakov R, et al. (2008) A new role for ns polyprotein cleavage in Sindbis virus replication. J Virol 82(13):6218-6231). This has provided an opportunity to exploit the under used ns proteins for amplification of the subgenomic RNAs encoding proteins of interest, which is normally transcribed from the subgenomic promoter and is not further amplified.

In some embodiments, a fragment of the nsP1 of the self-replicating RNA molecule of the disclosure is duplicated downstream of the 5′-UTR and upstream of the DLP.

In some embodiments, the first 193 nucleotides of nsP1 are duplicated downstream of the 5′ UTR and upstream of the DLP.

In some embodiments, the self-replicating RNA molecule comprises a modified 5′ untranslated region (5′-UTR). For example, the modified 5′-UTR can comprise one or more nucleotide substitutions at position 1, 2, 4, or a combination thereof. Preferably, the modified 5′-UTR comprises a nucleotide substitution at position 2, more preferably the modified 5′-UTR has a U->G substitution at position 2. Examples of such self-replicating RNA molecules are described in US Patent Application Publication US2018/0104359 and International Patent Application Publication WO2018075235, the content of which is incorporated herein by reference in its entirety.

In some embodiments, the UTRs can be wild type New World or Old World alphavirus UTR sequences, or a sequence derived from any of them. The 5′ UTR can be of any suitable length, such as about 60 nt or 50-70 nt or 40-80 nt. In some embodiments the 5′ UTR can also have conserved primary or secondary structures (e.g. one or more stem-loop(s)) and can participate in the replication of alphavirus or of replicon RNA. The 3′ UTR can be up to several hundred nucleotides, for example it can be 50-900 or 100-900 or 50-800 or 100-700 or 200 nt-700 nt. The '3 UTR also can have secondary structures, e.g. a step loop, and can be followed by a polyadenylate tract or poly-A tail.

The 5′ and 3′ untranslated regions can be operably linked to any of the other sequences encoded by the replicon. The UTRs can be operably linked to a promoter and/or sequence encoding a protein or peptide by providing sequences and spacing necessary for recognition and transcription of the other encoded sequences.

The GOI (e.g. the polynucleotides encoding the TCR polypeptides of the disclosure) can be expressed under the control of a subgenomic promoter. In certain embodiments, instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71, or hepatitis C viruses. Subgenomic promoters range from 24 nucleotides (Sindbis virus) to over 100 nucleotides (Beet necrotic yellow vein virus) and are usually found upstream of the transcription start.

The self-replicating RNA molecules can have a 3′ poly-A tail. It can also include a poly-A polymerase recognition sequence (e.g. AAUAAA) near its 3′ end.

In those instances where the self-replicating RNA molecule is to be packaged into a recombinant alphavirus particle, it can contain one or more sequences, so-called packaging signals, which serve to initiate interactions with alphavirus structural proteins that lead to particle formation. In some embodiments, the alphavirus particles comprise RNA derived from one or more alphaviruses, and structural proteins wherein at least one of said structural proteins is derived from two or more alphaviruses.

In some embodiments, the self-replicating RNA molecule comprises a VEEV derived vector wherein the structural viral proteins (e.g. nucleocapsid protein C, and envelope proteins P62, 6K, and E1) are removed and replaced by the coding sequence of the polypeptides of the disclosure.

### Engineered Cells Expressing the TCRs of the Disclosure

Engineered cells expressing the TCRs that specifically bind the CALR and JAK2 epitopes of the disclosure are within the scope of the disclosure. The present disclosure provides engineered cells (e.g., T cells) comprising any one of an alpha TCR chain and any one of a beta TCR chain, as disclosed in Tables 4 and 5. These engineered cells can be used to treat various disorders or disease, such as cancer, myeloproliferative disease and cardiovascular disease.

Suitable engineered cells include those obtained from, for example, animals and humans.

In some embodiments, the cell comprising the TCR of the disclosure is a T cell, a natural killer (NK) cell, a cytotoxic T lymphocyte (CTL), a regulatory T cell (Treg), a human embryonic stem cell, a lymphoid progenitor cell, a T cell-precursor cell, a pluripotent stem cell or induced pluripotent stem cell (iPSC) from which lymphoid cells may be differentiated. In some embodiments, the isolated cell comprising the TCR of the disclosure is a T cell. The T cell may be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from any source, including bone marrow, blood, lymph node, thymus, or other tissues or fluids. T cells may also be enriched for or purified. The T cell may be a human T cell. The T cell may be a T cell isolated from a human. The T cell can be any type of T cell and may be of any developmental stage, including, CD4+CD8+ double positive T cells, CD8+ T cells (e.g., cytotoxic T cells), CD4+ helper T cells, e.g., Th1 and Th2 cells, peripheral blood mononuclear cells (PBMCs), peripheral blood leukocytes (PBLs), tumor infiltrating cells, memory T cells, naïve T cells, and the like. The T cell may be a CD8+ T cell or a CD4+ T cell. The T cell may be an αβ T cell or a γδ T cell.

In some embodiments, the isolated cell comprising the TCR of the disclosure is a NK cell.

In some embodiments, the isolated cell comprising the TCR of the disclosure is a αβ T cell.

In some embodiments, the isolated cell comprising the TCR of the disclosure is a γδ T cell.

In some embodiments, the isolated cell comprising the TCR of the disclosure is a CD8+ cytotoxic T lymphocyte

In some embodiments, the isolated cell comprising the TCR of the disclosure is a human embryonic stem cell.

In some embodiments, the isolated cell comprising the TCR of the disclosure is a lymphoid progenitor cell.

In some embodiments, the isolated cell comprising the TCR of the disclosure is a pluripotent stem cell.

In some embodiments, the isolated cell comprising the TCR of the disclosure is an induced pluripotent stem cell (iPSC).

In some embodiments, the isolated cell comprising the TCR of the disclosure is a CD4+ T lymphocyte. In certain embodiments, the CD4+ T lymphocyte is a Th₁ cell. In certain embodiments, the CD4+ T lymphocyte is a Th2 cell. In certain embodiments, the CD4+ T lymphocyte is a Th17 cell. In certain embodiments, the CD4+ T lymphocyte is a T_{reg} cell. In certain embodiments, the CD4+ T lymphocyte is a T_{FH} cell

### Methods of Generating Engineered Cells Expressing the TCRs of the Disclosure

Described herein are methods for generating engineered cells comprising any one of an alpha TCR chain and any one of a beta TCR chain, as disclosed in Tables 4 and 5.

In some embodiments, to produce an engineered cell for use as a cell therapy product, cells, preferably autologous T cells, are obtained from a pheresis sample withdrawn from a subject and are modified with a vector of the disclosure that encodes the desired TCR alpha and beta sequences. When encountered by the engineered cells, targeted cells may be killed by the engineered cells by virtue of the ability of the engineered cells, such as engineered T cells, to exhibit specific target cell cytotoxicity (i.e., specific cell killing).

The pheresis sample may be collected from the subject by any of a number of suitable lymphocytapheresis, lymphapheresis, and leukaphoresis procedures now known or that become available in the art, which provide for the collection of peripheral blood leukocytes (PBLs) from collected peripheral blood, and from which leukocytes may be separated from other plasma components of the sample. Exemplary procedures are illustrated in, e.g., U.S. Patent Application Publication Numbers US 2004/0173778 and US 200/40147021, and U.S. Pat. Nos. 4,690,915, 5,126,132, 6,255,073, 5,846,827, 6,251,385, 6,225,044, 6,210,963, 6,194,207, 5,443,983, 6,040,177, 5,766,920, 6,210,662, 6,204,058, and 6,227,368.

Selected naive cells, preferably naïve T cells, may be substantially separated from non-selected pheresis sample components. In certain embodiments, naive T cells, which may be naive CD4+ T cells, naive CD8+ T cells, or naive CD4+ T cells and naive CD8+ T cells, are substantially separated from other PBLs, e.g., non-T cells. Methods for selection of PBLs include procedures employing Ficoll gradients, technique employing immunopurification (e.g., monoclonal antibodies directed against cell surface markers, such as CD molecules, and beads, such as Sepharose-, Protein A-, and Protein G-conjugated beads to which the antibodies may be adsorbed, and magnetic beads to which antibodies may be adsorbed), flow cytometry, and fluorescence-activated cell sorter (FACS) analysis.

In certain embodiments, selected naive T cells are substantially purified by magnetic bead purification systems such as those available in the art, e.g., Miltenyi beads (Myltenyi Biotec) and Dynabead systems (Dynal Biotech) combined with cell sorting procedures, such as FACS-based methods, or other appropriate cell sorting devices and methodologies.

A subject from whom a pheresis product comprising naive cells may be obtained is preferably a mammal in need of treatment, such as a dog, a cat, a horse, a rat, rabbit, mouse, a non-human primate, or a human. More preferably, the subject is a human patient in need of treatment for a cancer, a myeloproliferative disease, or a cardiovascular disease. Alternatively, in appropriate circumstances immune cells, such as naive T cells, that are not derived from a subject to be treated, but which are derived from another compatible source such as an immune cell donor, or even an immortalized or transformed immune cell line, may be employed to prepare cell therapy products in accordance with the invention.

The thus-selected naive cells, preferably naïve T cells, are then modified introducing a viral vector comprising any of the disclosed polynucleotides into the cells using known methods. The cells are able to replicate in vivo, resulting in long-term persistence that can lead to sustained tumor control.

In some embodiments, the naive cells, preferably naïve T cells, are transduced with any of the disclosed TCRs, resulting in constitutive expression of the TCR on the surface of the cell. The cells expressing the TCR may further be engineered to express one or more co-stimulatory molecules. Exemplary co-stimulatory molecules are CD28, ICOS, LIGHT, GITR, 4-1BB and OX40. The cells expressing the TCR may further be engineered to produce one or more cytokines or chemokines or proinflammatory mediators, such as TNFα, IFNγ, IL-2, IL-3, IL-6, IL-7, IL-11, IL-12, IL-15, IL-17 or IL-21. The cells may have their endogenous TCR locus and/or HLA locus inactivated using known gene editing technologies.

The engineered cells may optionally be expanded to produce therapeutic compositions and cell therapy products comprising engineered cells of a desired phenotype and number. An exemplary T cell expansion procedure includes incubating activated T cells with irradiated non-CD8+ cells in the presence of selected cytokines and an anti-CD3 antibody preparation to promote non-specific activated T cell expansion. Selection of the expansion protocols to be employed are within the purview of the artisan and may be facilitated by guidance in the art.

### Compositions

The disclosure also provides compositions comprising any of the polynucleotides, any of the polypeptides, or any of the vectors disclosed herein. In some embodiments, the compositions may comprise a vector comprising any of the nucleotides disclosed herein.

The compositions may comprise or may be formulated into a pharmaceutical composition comprising the composition and a pharmaceutically acceptable excipient. “Pharmaceutically acceptable” refers to the excipient that at the dosages and concentrations employed, will not cause unwanted or harmful effects in the subjects to which they are administered and include carrier, buffers, stabilizers, or other materials well known to those skilled in the art. The precise nature of the carrier or other material may depend on the route of administration, e.g., intramuscular, subcutaneous, oral, intravenous, cutaneous, intramucosal (e.g., gut), intranasal or intraperitoneal routes. Liquid carriers such as water, petroleum, animal or vegetable oils, mineral oil or synthetic oil may be included. Physiological saline solution, dextrose or other saccharide solution or glycols such as ethylene glycol, propylene glycol or polyethylene glycol may be included. Exemplary formulation are the Adenovirus World Standard (Hoganson et al, 2002): 20 mM Tris pH 8, 25 mM NaCl, 2.5% glycerol; or 20 mM Tris, 2 mM MgCl2, 25 mM NaCl, sucrose 10% w/v, polysorbate-80 0.02% w/v; or 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol. Many other buffers can be used, and examples of suitable formulations for the storage and for pharmaceutical administration of purified pharmaceutical preparations are known.

### Kits/Articles of Manufacture

Kits and articles of manufacture are also described. Such kits include a package or container that is compartmentalized to receive one or more dosages of the pharmaceutical compositions disclosed herein. Suitable containers include, for example, bottles. In one embodiment, the containers are formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert.

In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions provided herein are also prepared, placed in an appropriate container, and labeled for treatment of an indicated condition.

### Method of Treatment or Use

Provided herein are methods for treating a subject with any of the disclosed compositions or engineered cells. The compositions and engineered cells of the disclosure may be used to treat, prevent, or reduce the risk of a clinical condition.

In some embodiments, the clinical condition is a cancer, a myeloproliferative disease, or a cardiovascular disease.

In some embodiments, the myeloproliferative disease is selected from primary myelofibrosis (MPN), polycythemia vera (PV), essential thrombocythemia (ET), primary myelofibrosis (PFM), secondary myelofibrosis, acute myeloid leukemia (AML), secondary AML, chronic myelogenous leukemia (CML), clonal hematopoiesis of indeterminate potential (CHIP), and chronic myelomonocytic leukemia (CMML).

In some embodiments, the cancer is selected from lung cancer, lymphoid cancer, acute lymphoid leukemia, acute myeloid leukemia, chronic myelogenous leukemia, Burkitt's lymphoma, Hodgkin's lymphoma, plasma cell myeloma, biliary tract cancer, bladder cancer, liver cancer, pancreatic cancer, prostate cancer, skin cancer, thyroid cancer, stomach cancer, large intestine cancer, colon cancer, urinary tract cancer, central nervous system cancer, neuroblastoma, kidney cancer, breast cancer, cervical cancer, testicular cancer, and soft tissue cancer.

In some embodiments, the cardiovascular disease is selected from an acute coronary syndrome, an ischemic cerebrovascular disease, an ischemic heart disease, a thrombosis, a venous thromboembolism, a deep vein thrombosis, a pulmonary embolism, a catastrophic intra-abdominal thromboses, a peripheral arterial disease, a hypertension, a heart failure, an atrial fibrillation, a coronary heart disease, an atherosclerosis, and a clonal hematopoiesis.

In some embodiments, the methods comprise administering to the subject in need thereof a composition comprising any of the polynucleotides, any of the polypeptides, or any of the vectors disclosed herein. In some embodiments, the compositions may comprise a vector comprising any of the nucleotides disclosed herein.

In some embodiments, the methods comprise administering to the subject in need thereof a composition comprising any of the polynucleotides disclosed herein, wherein the polynucleotides encode a TCR comprising an alpha chain and a beta chain, wherein the alpha chain comprises a CDR3 comprising an amino acid sequence provided in Table 4, and the corresponding beta chain comprises a CDR3 comprising a corresponding amino acid sequence provided in Table 5. In some embodiments, the methods disclosed herein comprise administering to the subject in need thereof a composition comprising polynucleotides that encode a TCR comprising an alpha chain and a beta chain, wherein the alpha chain comprises a CDR3 that is encoded by a nucleic acid sequence provided in Table 4, and the corresponding beta chain comprises a CDR3 that is encoded by a corresponding nucleic acid sequence provided in Table 5 (i.e. the beta chain CDR3 provided in Table 5 is in the same row number (row 1, for example) as the alpha chain CDR3 provided in Table 4 (row 1, for example)). For the sake of brevity, each possible alternative is not parsed out, but each constitute as separate embodiments as if fully described.

In some embodiments, the methods comprise administering to the subject in need thereof a composition comprising polynucleotides that encode a TCR comprising an alpha chain and a beta chain, wherein the alpha chain comprises a CDR1 comprising the amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5. In some embodiments, the methods comprise administering to the subject in need thereof a composition comprising polynucleotides that encode a TCR comprising an alpha chain and a beta chain, wherein the alpha chain comprises a CDR1 that is encoded by a nucleic acid sequence provided in Table 4, a CDR2 that is encoded by a nucleic acid sequence provided in Table 4, and a CDR3 that is encoded by a nucleic acid sequence provided in Table 4, and the corresponding beta chain comprises a CDR1 that is encoded by a nucleic acid sequence provided in Table 5, a CDR2 that is encoded by a nucleic acid sequence provided in Table 5, and a CDR3 that is encoded by a nucleic acid sequence provided in Table 5 (i.e. the beta chain CDRs provided in Table 5 are in the same row number (row 1, for example) as the alpha chain CDRs provided in Table 4 (row 1, for example)). For the sake of brevity, each possible alternative is not parsed out, but each constitute separate embodiments as if fully described.

In some embodiments, the methods comprise administering to the subject in need thereof a composition comprising polynucleotides that encode a TCR comprising an alpha chain and a beta chain, wherein the alpha chain comprises a VJ region comprising an amino acid sequence that is at least 90%, 95%, 96%, 97%, 98%, 99% or 100% identical to an amino acid sequence provided in Table 4, and the corresponding beta chain comprises a VDJ region comprising an amino acid sequence that is at least 90%, 95%, 96%, 97%, 98%, 99% or 100% identical to an amino acid sequence provided in Table 5. In some embodiments, the methods comprise administering to the subject in need thereof a composition comprising polynucleotides that encode a TCR comprising an alpha chain and a beta chain, wherein the alpha chain comprises a VJ region that is encoded by a nucleic acid sequence that is at least 90%, 95%, 96%, 97%, 98%, 99% or 100% identical to a nucleic acid sequence provided in Table 4, and the corresponding beta chain comprises a VDJ region that is encoded by a nucleic acid sequence that is at least 90%, 95%, 96%, 97%, 98%, 99% or 100% identical to a nucleic acid sequence provided in Table 5 (i.e. the beta chain VDJ region provided in Table 5 is in the same row number (row 1, for example) as the alpha chain VJ region provided in Table 4 (row 1, for example)). For the sake of brevity, each possible alternative is not parsed out, but each constitute separate embodiments as if fully described.

In some embodiments, the methods comprise administering to the subject in need thereof a composition comprising polynucleotides that encode a TCR comprising an alpha chain and a beta chain, wherein the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5. Also described herein are polynucleotides that encode a TCR comprising an alpha chain and a beta chain, wherein the alpha chain is encoded by a nucleic acid sequence that is at least 90%, 95%, 96%, 97%, 98%, 99% or 100% identical to a nucleic acid sequence provided in Table 4, and the corresponding beta chain is encoded by a nucleic acid sequence that is at least 90%, 95%, 96%, 97%, 98%, 99% or 100% identical to a nucleic acid sequence provided in Table 5 (i.e. the beta chain provided in Table 5 is in the same row number (row 1, for example) as the alpha chain provided in Table 4 (row 1, for example)). For the sake of brevity, each possible alternative is not parsed out, but each constitute separate embodiments as if fully described.

In some embodiments, the methods comprise administering to the subject in need thereof a composition comprising a viral vector comprising a polynucleotide encoding any of the TCR polypeptides of the disclosure. In any of the methods disclosed herein, the composition that is administered to a subject may comprise a vector selected from an adenovirus vector, an alphaviral vector, a poxvirus vector, an adeno-associated virus vector, a retrovirus vector, a self-replicating RNA molecule, and a combination thereof. In some embodiments, the vector is selected from Ad26 vector, MVA vector, GAd20 vector, a self-replicating RNA molecule, and combinations thereof.

In some embodiments, the methods comprise administering to the subject in need thereof a composition comprising any of the polypeptides disclosed herein, wherein the polypeptides are TCRs. In some embodiments, the methods comprise administering to the subject in need thereof a composition comprising a TCR comprising an alpha chain and a beta chain, wherein the alpha chain comprises a CDR3 comprising an amino acid sequence provided in Table 4, and the beta chain comprises a CDR3 comprising a corresponding amino acid sequence provided in Table 5. (i.e. the beta chain CDR3 provided in Table 5 is in the same row number (row 1, for example) as the alpha chain CDR3 provided in Table 4 (row 1, for example)). For the sake of brevity, each possible alternative is not parsed out, but each constitute separate embodiments as if fully described.

In some embodiments, the methods comprise administering to the subject in need thereof a composition comprising a TCR comprising an alpha chain and a beta chain, wherein the alpha chain comprises a CDR1 comprising an amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5 (i.e. the beta chain CDRs provided in Table 5 are in the same row number (row 1, for example) as the alpha chain CDRs provided in Table 4 (row 1, for example)). For the sake of brevity, each possible alternative is not parsed out, but each constitute separate embodiments as if fully described.

In some embodiments, the methods comprise administering to the subject in need thereof a composition comprising a TCR comprising an alpha chain and a beta chain, wherein the alpha chain comprises a variable and joining (VJ) region comprising an amino acid sequence that is at least 90%, 95%, 96%, 97%, 98%, 99% or 100% identical to an amino acid sequence provided in Table 4, and the corresponding beta chain comprises a variable, diversity and joining (VDJ) region comprising an amino acid sequence that is at least 90%, 95%, 96%, 97%, 98%, 99% or 100% identical to an amino acid sequence provided in Table 5 (i.e. the beta chain VDJ region provided in Table 5 is in the same row number (row 1, for example) as the alpha chain VJ region provided in Table 4 (row 1, for example)). For the sake of brevity, each possible alternative is not parsed out, but each constitute separate embodiments as if fully described.

In some embodiments, the methods comprise administering to the subject in need thereof a composition comprising a TCR comprising an alpha chain and a beta chain, wherein the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5 (i.e. the beta chain provided in Table 5 is in the same row number (row 1, for example) as the alpha chain provided in Table 4 (row 1, for example)). For the sake of brevity, each possible alternative is not parsed out, but each constitute separate embodiments as if fully described.

In some embodiments, the methods comprise administering to the subject in need thereof any of the engineered cells disclosed herein. In certain embodiments, the engineered cells comprise any one of an alpha TCR chain and any one of a beta TCR chain, as disclosed in Tables 4 and 5. For the sake of brevity, each possible alternative is not parsed out, but each constitute separate embodiments as if fully described.

In some embodiments, the methods comprise administering to the subject in need thereof an engineered cell comprising any of the polypeptides disclosed herein, wherein the polypeptides are TCRs. In some embodiments, the methods comprise administering to the subject in need thereof an engineered cell comprising a TCR comprising an alpha chain and a beta chain, wherein the alpha chain comprises a CDR3 comprising an amino acid sequence provided in Table 4, and the beta chain comprises a CDR3 comprising a corresponding amino acid sequence provided in Table 5. (i.e. the beta chain CDR3 provided in Table 5 is in the same row number (row 1, for example) as the alpha chain CDR3 provided in Table 4 (row 1, for example)). For the sake of brevity, each possible alternative is not parsed out, but each constitute separate embodiments as if fully described.

In some embodiments, the methods comprise administering to the subject in need thereof an engineered cell comprising a TCR comprising an alpha chain and a beta chain, wherein the alpha chain comprises a CDR1 comprising an amino acid sequence provided in Table 4, a CDR2 comprising an amino acid sequence provided in Table 4, and a CDR3 comprising an amino acid sequence provided in Table 4, and the corresponding beta chain comprises a CDR1 comprising an amino acid sequence provided in Table 5, a CDR2 comprising an amino acid sequence provided in Table 5, and a CDR3 comprising an amino acid sequence provided in Table 5 (i.e. the beta chain CDRs provided in Table 5 are in the same row number (row 1, for example) as the alpha chain CDRs provided in Table 4 (row 1, for example)). For the sake of brevity, each possible alternative is not parsed out, but each constitute separate embodiments as if fully described.

In some embodiments, the methods comprise administering to the subject in need thereof an engineered cell comprising a TCR comprising an alpha chain and a beta chain, wherein the alpha chain comprises a variable and joining (VJ) region comprising an amino acid sequence that is at least 90%, 95%, 96%, 97%, 98%, 99% or 100% identical to an amino acid sequence provided in Table 4, and the corresponding beta chain comprises a variable, diversity and joining (VDJ) region comprising an amino acid sequence that is at least 90%, 95%, 96%, 97%, 98%, 99% or 100% identical to an amino acid sequence provided in Table 5 (i.e. the beta chain VDJ region provided in Table 5 is in the same row number (row 1, for example) as the alpha chain VJ region provided in Table 4 (row 1, for example)). For the sake of brevity, each possible alternative is not parsed out, but each constitute separate embodiments as if fully described.

In some embodiments, the methods comprise administering to the subject in need thereof an engineered cell comprising a TCR comprising an alpha chain and a beta chain, wherein the alpha chain comprises an amino acid sequence that is at least 90%, 95%, 96%, 97%, 98%, 99% or 100% identical to an amino acid sequence provided in Table 4, and the corresponding beta chain comprises an amino acid sequence that is at least 90%, 95%, 96%, 97%, 98%, 99% or 100% identical to an amino acid sequence provided in Table 5 (i.e. the beta chain provided in Table 5 is in the same row number (row 1, for example) as the alpha chain provided in Table 4 (row 1, for example)). For the sake of brevity, each possible alternative is not parsed out, but each constitute separate embodiments as if fully described.

In some embodiments, the engineered cell comprising the TCR is a T cell, a natural killer (NK) cell, a cytotoxic T lymphocyte (CTL), a regulatory T cell (Treg), a human embryonic stem cell, a lymphoid progenitor cell, a T cell-precursor cell, a pluripotent stem cell or induced pluripotent stem cell (iPSC) from which lymphoid cells may be differentiated.

In certain embodiments, the engineered cell is derived from naïve cells that are obtained from a subject in need of treatment. In certain embodiments, the engineered cell is not derived from a subject in need of treatment, but is instead derived from another compatible source such as an immune cell donor, or an immortalized or transformed immune cell line.

In some embodiments, desired amounts of engineered cells are employed to prepare a cell therapy product for therapeutic administration. In certain embodiments, the cell therapy product is reinfused or transfused back into the subject from whom the pheresis sample used to derive the cell therapy product was obtained. Reinfusion procedures that may be employed include those procedures disclosed in, for example, U.S. Pat. Nos. 4,844,893 and 4,690,915. In certain embodiments, the cell therapy product is reinfused or transfused back into a different subject from whom the pheresis sample used to derive the cell therapy product was obtained.

In some embodiments, prior to administration one or more quality assurance tests are performed on the activated T lymphocytes or cell therapy product. In some embodiments, the quality assurance testing comprises performing one or more tests to confirm: HLA match between patient and T lymphocytes; flow cytometry analysis (CD8+, TCR+); sterility (no bacterial or fungal growth); gram-stain negative for bacteria; mycoplasma negative for PCR/ELISA; no residual Drosophila DNA; absence of insect virus cDNA; viability (>72% viable); and cytolytic activity by CTL assay.

### Dose and Route of Administration

In some embodiments, desired amounts of viral vectors are employed to prepare a composition for therapeutic administration.

In some embodiments, the compositions comprising viral vectors is administered at a dose from about 1×10⁴ IFU (Infectious Unit) to about 1×10¹¹ IFU per dose. In certain embodiments, the viral vector is an adenovirus vector, an Ad26 vector, a GAd20 vector, a poxvirus vector or an MVA vector.

In some embodiments, the compositions comprising self-replicating RNA molecule is administered at a dose from about 1 microgram to about 100 micrograms.

In some embodiments, the compositions disclosed herein may be administered to a subject by a variety of routes such as subcutaneous, topical, oral and intramuscular. Administration of the compositions may be accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present disclosure also has the objective of providing suitable topical, oral, systemic and parenteral formulations for use in the methods of prophylaxis and treatment.

In some embodiments, intramuscular administration of the composition can be achieved by using a needle. An alternative is the use of a needleless injection device to administer the composition (using, e.g., Biojector™) or a freeze-dried powder containing the composition. For intravenous, cutaneous or subcutaneous injection, or injection at the site of affliction, the composition may be the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation may also be employed.

The actual amount administered, and rate and time-course of administration, will depend on the nature and severity of what is being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

In some embodiments, desired amounts of engineered cells are employed to prepare a cell therapy product for therapeutic administration.

To treat a subject, an effective amount of a cell therapy product is administered to a subject suffering from or diagnosed as having a disease, disorder, or condition. The subject can be a human patient in need of treatment for a cancer, a myeloproliferative disease, or a cardiovascular disease.

Effective amounts or doses of the cell therapy products may be ascertained by routine methods such as modeling, dose escalation studies or clinical trials, and by taking into consideration routine factors, e.g., the mode or route of administration or product delivery, the pharmacokinetics of the cell therapy product, the severity and course of the disease, disorder, or condition, the subject's previous or ongoing therapy, the subject's health status and response to drugs, and the judgment of a treating physician. As exemplary dosage amounts, cell populations may comprise from about 1×10⁶ to about 1×10¹² activated T cells, such as 1×10⁸ to 1×10¹¹ or 1×10⁹ to 1×10¹⁰ activated T cells for an adult human.

The cell therapy product can be prepared as a therapeutic composition comprising engineered cells, preferably engineered T cells, and a vehicle suitable for the maintenance of the engineered cells until they are infused into the subject, such as a pharmaceutically acceptable diluent or solvent.

Any suitable technique for administering compositions comprising cellular components into a subject may be employed. For example, administration of the engineered T cell via intravenous infusion may be employed. Multiple infusions may be required or indicated, and these infusions may occur over a period of several weeks or longer. Exemplary techniques are described in, for example, U.S. Pat. Nos. 4,844,893 and 4,690,915.

### EXAMPLES

These examples are provided for illustrative purposes only and not to limit the scope of the claims provided herein.

### Example 1. TCR Sequence Analysis

### 12 Day Assay—Transduction, Endogenous, Exogenous—10× Endpoint

Single cell TCR α/β and RNA sequencing was performed on CD8+ T cell samples isolated from a leukopak from four healthy human donors. Dendritic cells from each donor were treated with the following: a) DMSO treatment or exogenously loaded single 9-mer peptide corresponding to one of two mutJAK2 epitopes (“exogenous treatment”), b) transfection with a tandem minigene construct composed of the combined mutJAK2 and mutCALR epitopes (mutCALR type I and mutCALR type II) (“endogenous treatment”), or c) transduction with an Ad5 virus containing the mutJAK2 and mutCALR antigen construct (“Ad5 treatment”).

The 9-mer peptides corresponding to two mutJAK2 epitopes have the following amino acid sequences: VLNYGVCFC (SEQ ID NO: 681) and FCGDENILV (SEQ ID NO: 682). The tandem minigene construct composed of the combined mutJAK2 and mutCALR epitopes has the amino acid sequence:

(SEQ ID NO: 683)
MKDKQDEEQRTRRMMRTKMRMRRMRRTRRKMRRKMSPARPRTSCREACLQ
GWTEAAYEEAEDNCRRMMRTKAAYVLNYGVCFCAAYFCGDENILV.

In SEQ ID NO: 683, the mutCALR type 1 sequence is underlined, the mutCALR type II sequence is underlined and bold, and the two mutJAK2 sequences are underlined an italics.

The mutJAK2 and mutCALR antigen construct (also known as LS_CALR_JAK2-2×9mer (HEME002)) that is contained within the Ad5 virus has the following amino acid sequence:

(SEQ ID NO: 702)
MACPGFLWALVISTCLEFSMAMKDKQDEEQRTRRMMRTKMRMR
RMRRTRRKMRRKMSPARPRTSCREACLQGWTEAAYEEAEDNCR
RMMRTKAAYVLNYGVCFCAAYFCGDENILV.

Subsequently, autologous pan-T cells were added to the co-culture to initiate TCR stimulation.

In all cases, the TCRs were stimulated for 11 days, followed by overnight re-stimulation treatment with individual peptides. Cells were harvested and CD8+ T cells were isolated using negative selection magnetic bead capture techniques. These isolated CD8+ T cells were analyzed using the 10× genomics single cell sequencing platform.

### Procedure

Day −2 (for Ad5 treatment) or Day −1 (for exogenous and endogenous treatment): Donor dendritic cells (DC) from the four healthy human donors described, above, were thawed in IMDM5, (IMDM5 Media: Gibco™, 12440 supplemented with 5% human antibody serum Sigma, H3667) with 1× anti-aggregate (C.T.L., CTL-AA-001). The cells were washed by centrifuging at 1300 rpm for five minutes and resuspended in IMDM5 containing 50 ng/mL GM-CSF and IL-4. The cells were plated into 6 well plates at ˜1-1.5e6 cells/well in 2 mL IMDM5 and incubated overnight at 37° C./5% CO2.

Day −1 (for Ad5 treatment): DCs were removed from plates, and wells of same donors were combined. Cells were washed in IMDM5 and counted and resuspended at a density of 6e5 cells/mL. 50 μL of cells were seeded (for a density of 3e4 cells/well) to appropriate wells of clear, round bottom 96 well plates according to plate layout. At least one row/column was left empty around outside edges of plates and filled with 100 μL DPBS. In a BSL2+ environment, Ad5 constructs were diluted in IMDM5 media with 2× (final concentration: 5 μg/mL) Polybrene (Millipore™, Cat #: TR-1003-G) to concentrations of 5,000 Multiplicity of Infection (MOI) per 50 μL. 50 μL of each construct was added to the appropriate wells of DCs according to plate layout. Plates were returned to incubator at 37° C./5% CO2 overnight.

Day 0 (for endogenous treatment): DCs were removed from the plates, and wells of same donors were combined. Cells were washed in IMDM5 and counted and resuspended at a density of 5e5 cells/mL. 500 μL of cells were seeded (for a density of 2.5e5 cells/well) to appropriate wells of 24 well plates according to plate layout. Following NanoJuice™ Kit Protocol, (Millipore Sigma™, 71902), Opti-MEM™ was combined with core transfection reagent and transfection booster and incubated at room temperature (RT) for five minutes. DNA was added to the appropriate tubes and mixed by gentle pipetting. Tubes were incubated at RT for 15 minutes. 20 μL of each preparation was added dropwise to the appropriate wells as indicated in plate layout. Plates were rocked gently to mix and incubated at 37° C./5% CO2 for six hours. Wells were transferred from endogenous transfection pates to microcentrifuge tubes. Tubes were centrifuged at 1500×g for five minutes. Cells were resuspended in 500 μL DPBS. Washes were repeated for a total of two washes. After the final wash, cells were resuspended in 500 μL IMDM5. Cells were pipetted to mix and 65 μL was transferred to appropriate wells indicated in the plate layout. The corresponding donor PanT cells (i.e. the PanT cells from the same donor as the dendritic cells) were thawed in IMDM5 with 1× anti-aggregate and washed by centrifuging at 1300 rpm for five minutes. Cells were resuspended in IMDM5 at 3e6 cells/mL. 100 μL of prepared PanT donor cells was added to corresponding donor DC wells according to plate layout (for a density of 3e5 PanT cells/well. Final ratio 1:10 DC:PanT). 200 μL of DPBS was added to outer wells of the plate(s). Plates were returned to incubator at 37° C./5% CO2.

Day 0 (for exogenous treatment): DCs were removed from plates, and wells of same donors were combined. Cells were washed in IMDM5 and counted and resuspended at a density of 3e5 cells/mL. 100 μL of cells were seeded (for a density of 3e4 cells/well) to appropriate wells in clear 96 well round bottom plate(s) as indicated in the plate layout. Peptides were diluted to 20× final concentrations in IMDM5 and 5 μL of the diluted peptides were added to appropriate exogenous wells according to the plate layout. 200 μL DPBS was added to outside wells and incubated at 37° C./5% CO₂ for one and a half to two hours. After incubation, plates were placed in a room temperature irradiator for a calculated time to achieve the desired dose of 50 grays of radiation. Corresponding donor PanT cells were thawed in IMDM5 with 1× anti-aggregate and washed by centrifuging at 1300 rpm for five minutes. Cells were resuspended in IMDM5 at 3e6 cells/mL. 100 μL of prepared PanT donor cells were added to corresponding donor DC wells according to plate layout (for a density of 3e5 PanT cells/well. Final ratio 1:10 DC:PanT). The plates were returned to the incubator at 37° C./5% CO₂.

Day 0 (for Ad5 treatment): After the plates were incubated with Ad5 for ˜20 hours they were removed from the incubator. The plates were washed by centrifuging at 300×g for five minutes and pellets were resuspended in DPBS (Gibco™, 14140). Washes were repeated for a total of three washes. After the final wash, cells were resuspended in 100 μL IMDM5. Corresponding donor PanT cells were thawed in IMDM5 with 1× anti-aggregate. Cells were washed by centrifuging at 1300 rpm for five minutes and then were resuspended in IMDM5 at 3e6 cells/mL. 100 μL of prepared PanT donor cells were added to corresponding donor DC wells according to the plate layout (for a density of 3e5 PanT cells/well. Final ratio 1:10 DC:PanT). 200 μL of DPBS was added to outer wells of plates. Plates were returned to the incubator at 37° C./5% CO2.

Day 3 and following Day 3 (for all treatments): IMDM5 media was prepared with IL-2, IL-7, and IL-15 at 2× final concentrations. (Final concentrations: IL-2: 50 IU/mL, IL-7: 25 ng/mL, and IL-15: 25 ng/mL). 100 μL media was removed from each well and replaced with prepared IMDM5 cytokine media. Plates were gently rocked to mix and returned to the incubator at 37° C./5% CO₂. This protocol was repeated every 2-3 days after, up to day 12, with the exception that IL-2 concentration was increased to 100 IU/mL.

Day 11: (If restim fell on a day when media needs to be refreshed, refresh was performed first). Peptides were prepared corresponding to exogenous, endogenous, or Ad5 constructs in IMDM5 for the restim of the PanT cells. For single peptide restim a final concentration of 5 μg/mL was used. For the CALR peptide pool, each peptide was used at a final concentration of 1 μg/mL (peptides were pooled at 500× final concentration prior to adding to plates). DMSO control was prepared in IMDM5 to match the highest concentration of peptide used (5 μg/mL). 5 μL of each preparation were added to appropriate wells. The plates were gently rocked to mix. The plates were returned to the incubator and incubated overnight (˜18 hours) at 37° C./5% CO₂.

The sequences for the CALR peptide pool are provided in Table 1:

TABLE 1

| peptide | peptide | Sequence |
|---|---|---|
| name | sequences | Identifier |
| 1.2 | RPRTSCREA | SEQ ID NO: 684 |
| 1.3 | SPARPRTSC | SEQ ID NO: 685 |
| 1.4 | RKMSPARPR | SEQ ID NO: 686 |
| 1.5 | MRRKMSPAR | SEQ ID NO: 687 |
| 1.6 | KMRRKMSPA | SEQ ID NO: 688 |
| 1.7 | TRRKMRRKM | SEQ ID NO: 689 |
| 1.8 | RTRRKMRRK | SEQ ID NO: 690 |
| 1.9 | RRTRRKMRR | SEQ ID NO: 691 |
| 1.1 | RMRRTRRKM | SEQ ID NO: 692 |
| 1.11 | RRMRRTRRK | SEQ ID NO: 693 |
| 1.12 | MRRMRRTRR | SEQ ID NO: 694 |
| 1.14 | MRMRRMRRT | SEQ ID NO: 695 |
| 1.15 | KMRMRRMRR | SEQ ID NO: 696 |
| 1.17 | MRTKMRMRR | SEQ ID NO: 697 |
| 1.18 | MMRTKMRMR | SEQ ID NO: 698 |
| 1.19 | RMMRTKMRM | SEQ ID NO: 699 |
| 1.23 | RRMMRTKMR | SEQ ID NO: 700 |
| 1.25 | RTRRMMRTK | SEQ ID NO: 701 |

Day 12 (for all treatments): A vial of PanT cells were thawed from each donor (to be used as baselines) in IMDM5 with 1× anti-aggregate and washed by centrifuging at 1300 rpm for five minutes and resuspend in IMDM5. Replicates of each sample were combined into tubes on ice and centrifuged at 1500 rpm for five minutes at 4° C. Supernatant was removed and 300 μL running buffer was added to wash. The tubes were centrifuged again, and supernatant was removed. The antibody cocktail provided in the CD8 T cells negative selection bead kit was prepared, scaled accordingly (40 μL buffer and 10 μL antibody per sample). The antibody cocktail included: biotin-conjugated monoclonal anti-human antibodies against CD4, CD15, CD16, CD19, CD34, CD36, CD56, CD123, TCR γ/δ, and CD235a (Glycophorin A), MicroBeads conjugated to monoclonal anti-CD14 antibody (isotype: mouse IgG2a), anti-CD61 antibody (isotype: mouse IgG1), and anti-biotin antibody (isotype: mouse IgG1).

50 μL prepared antibody cocktail was added to each sample and incubated for five minutes on ice. Bead cocktail was prepared and scaled accordingly (30 μL buffer and 20 μL beads per sample). 50 μL was added to each sample and incubate for ten minutes on ice. 400 μL buffer was added to bring sample volumes to 500 μL. 400 μL buffer was added to baseline samples to bring volumes to 500 μL. The Manual CD8+ Separation protocol (MACS Miltenyi Biotec™, 130-096-495) was followed. Columns and tubes (15 mL) were set up and rinsed with 3 mL buffer to pre-wet. 500 μL of cells was added and flow through was collected. Cells were washed with 3 mL buffer and collected. Cells were counted and washed by centrifuging at 1500 rpm for five minutes. The cells were resuspended in PBS at 1e4 cells/mL.

### Single Cell V(D)J 10× Genomics Protocols

The Single Cell V(D)J (variable, diverse, and joining) 10× genomics protocols (Chromium™ Single Cell V(D)J Reagent Kits User Guide—CG000086) was used for measuring immune repertoire information and gene expression from the same cell. Cells obtained from the 12 Day Assay were washed and resuspended in 1× phosphate-buffered saline (PBS) containing 0.04% bovine serum albumin (BSA) to achieve target cell concentration (˜20,000 cells/sample). Gel Bead-in-Emulsions (GEMs) were generated by combining barcoded single cell VDJ 5′ Gel beads, a Master Mix with cells, and partitioning oil on a 10× Chromium™ Controller Single Cell A Chip. Following GEM generation, the Gel Bead dissolved, and any co-partitioned cell was lysed. Oligonucleotides containing sequencing primers, 10× cell barcode, 10× unique molecular identifier (UMI) barcode, and template switch oligo (TSO) were released and mixed with the cell lysate and Master Mix containing reverse transcription (RT) reagents. Incubation of GEM-RT produced barcoded full-length cDNA from mRNA. 10× barcoded full-length cDNA from GEM-RT was purified and amplified via polymerase chain reaction (PCR) (14 cycles) with primers against the 5′ and 3′ ends added during GEM-RT. Full Length VDJ segments were enriched from amplified cDNA with primers specific to TCR region. Enzymatic fragmentation and size selection were used to optimize the cDNA amplicon size prior to 5′ gene expression (GEX) library construction. P5 (AATGATACGGCGACCACCGA) (SEQ ID NO: 703), P7 (CAAGCAGAAGACGGCATACGAGAT) (SEQ ID NO: 704), a sample index (NNNNNNNN) (SEQ ID NO: 705), and Illumina™ R2 (AGATCGGAAGAGCACACGTCTGAACTCCAGTCAC) (SEQ ID NO: 706) sequence were added via End Repair, A tailing, Adaptor Ligation and Sample Index PCR. The final libraries contained the P5 and P7 priming sites used in Illumina™ Sequencing. Pooled/normalized libraries were sequenced at the recommended depth/run parameters. Libraries sequenced on NovaSeq™ produced high quality data with expected Q30 (sequencing Analysis Viewer) scores. Multiplexed sequences were de-multiplexed and analyzed using The Chromium™ Single Cell Immune Profiling Software Suite.

### Example 2. Identification of TCRs Enriched Upon Stimulation

### Identifying Expanded TCRs

To identify TCRs enriched due to antigen stimulation, T-cell receptor alpha/beta (TCR α/β) sequences from T-cell populations treated with either endogenous minigene or by Ad5 transduction were compared against corresponding sequences from T-cell populations obtained by stimulation against an empty minigene (i.e. no antigen) or an Ad5 null construct (i.e. Ad5 transfection without an antigen cassette). For TCRα/β sequences obtained from stimulation by exogenously loaded dendritic cells (DC's), the corresponding DMSO treated controls (i.e. DMSO+no antigen stimulation) were used. TCR α/β sequences present in cell populations enriched at a false discovery rate (FDR) of <0.0003 and a minimum of 2 fold increase in TCR proportions compared to controls were selected for further analysis. TCR α/β sequences that were highly abundant (˜1.0% or higher) both in the controls and treatment conditions and that were significantly enriched were also chosen for further examination.

Identifying Expanded TCR Sequences from Activated T Cells

To further triage the number of TCRs for experimental validation, single cell RNA (scRNA) sequencing data was obtained from the same cells. The T-cell activation markers considered for mRNA based gating of T cells were TNF, GZMB, CD69 and PRF1 (Perform). These markers and their cutoffs are listed in Table 2, below. TCRs which were both clonally expanded and present in cells with higher than the cutoff mRNA expression in all 4 genes were given the highest priority. In addition, TCRs that shared sequence identity with known self-antigens or viral antigens were eliminated from further consideration. The IEDB (www.iedb.org) public repository database was used to identify the self/viral antigen responsive TCR sequences.

TABLE 2
T cell marker genes and cutoffs

| | mRNA normalized |
|---|---|
| T cell Activation | expression cutoff |
| Marker Genes | (log units) |
| TNF, CD69, PRF1 | 0.5 |
| GZMB | 1.0 |

scRNA Analysis

The Seurat R package was used to perform the scRNA sequencing analysis. The samples were first quality controlled using high mitochondrial content (>5%), excessively low (<300) counts, and library size criteria. Cells that failed to pass quality control (QC) thresholds were removed. After QC filtering, the SCTransform algorithm was used to normalize and transform raw RNA read counts. Samples pre- and post-12 day expansion were combined using the sample Integration method implemented in Seurat. A principal component analysis (PCA), followed by uniform manifold approximation and projection (UMAP) clustering on the first 30 principal components was performed. The normalized mRNA data was then utilized, and the expression profiles of various T cell activation markers were examined to establish the cutoff criteria. The activation profiles showed a generally bimodal distribution, and the cutoff was chosen at the point of the trough between the two neighboring peaks of the bimodal distribution. See table 1 for cut-off values.

Table 3 provides donor number, treatment condition, the V(D)J genes, and the constant genes for thirty-four sequenced T-cell receptors.

TABLE 3

| | | | | Alpha Chain | | | | Beta Chain | | | |
|---|---|---|---|---|---|---|---|---|---|---|---|
| | | Treatment | | V | D | J | Constant | V | D | J | Constant |
| ID | Donor | Condition | Antigen | Genes | Genes | Genes | Genes | Genes | Genes | Genes | Genes |
| 1 | 274 | Ad5 | CALR | TRAV39 | — | TRAJ34 | TRAC | TRBV12-5 | TRBD1 | TRBJ2-7 | TRBC2 |
| 2 | 274 | Ad5 | CALR | TRAV3 | — | TRAJ22 | TRAC | TRBV2 | TRBD1 | TRBJ2-1 | TRBC2 |
| 3 | 118 | Ad5 | CALR | TRAV4 | — | TRAJ22 | TRAC | TRBV6-1 | TRBD1 | TRBJ1-1 | TRBC1 |
| 4 | 118 | Ad5 | JAK2ep2 | TRAV41 | — | TRAJ45 | TRAC | TRBV6-5 | TRBD2 | TRBJ2-7 | TRBC2 |
| 5 | 118 | Ad5 | JAK2ep2 | TRAV12-2 | — | TRAJ41 | TRAC | TRBV5-1 | TRBD1 | TRBJ1-1 | TRBC1 |
| 6 | 118 | Ad5 | JAK2ep2 | TRAV38- | — | TRAJ49 | TRAC | TRBV2 | TRBD1 | TRBJ2-7 | TRBC2 |
| | | | | 2DV8 | | | | | | | |
| 7 | 118 | Ad5 | JAK2ep2 | TRAV39 | — | TRAJ33 | TRAC | TRBV10-2 | TRBD1 | TRBJ2-7 | TRBC2 |
| 8 | 118 | Ad5 | CALR | TRAV12-2 | — | TRAJ13 | TRAC | TRBV6-5 | TRBD2 | TRBJ2-7 | TRBC2 |
| 9 | 274 | Ad5 | JAK2ep2 | TRAV1-1 | — | TRAJ4 | TRAC | TRBV7-6 | — | TRBJ2-7 | TRBC2 |
| 10 | 118 | Ad5 | JAK2ep1 | TRAV26-2 | — | TRAJ45 | TRAC | TRBV19 | — | TRBJ1-5 | TRBC1 |
| 11 | 644 | Ad5 | JAK2ep2 | TRAV1-2 | — | TRAJ29 | TRAC | TRBV7-9 | — | TRBJ2-2 | TRBC2 |
| 12 | 118 | Ad5 | JAK2ep1 | TRAV38- | — | TRAJ34 | TRAC | TRBV27 | TRBD2 | TRBJ2-7 | TRBC2 |
| | | | | 2DV8 | | | | | | | |
| 13 | 118 | Ad5 | CALR | TRAV12-3 | — | TRAJ45 | TRAC | TRBV7-3 | TRBD2 | TRBJ2-1 | TRBC2 |
| 14 | 118 | Ad5 | CALR | TRAV35 | — | TRAJ47 | TRAC | TRBV5-8 | — | TRBJ2-7 | TRBC2 |
| 15 | 118 | Exogenous | JAK2ep2 | TRAV2 | — | TRAJ43 | TRAC | TRBV27 | TRBD1 | TRBJ2-3 | TRBC2 |
| 16 | LP263 | Exogenous | JAK2ep1 | TRAV1-1 | — | TRAJ31 | TRAC | TRBV7-9 | TRBD2 | TRBJ2-1 | TRBC2 |
| 17 | LP263 | Exogenous | JAK2ep2 | TRAV1-2 | — | TRAJ43 | TRAC | TRBV7-9 | TRBD2 | TRBJ1-1 | TRBC1 |
| 18 | 644 | Ad5 | JAK2ep1 | TRAV38-1 | — | TRAJ58 | TRAC | TRBV7-9 | — | TRBJ2-7 | TRBC2 |
| 19 | 118 | Ad5 | JAK2ep2 | TRAV4 | — | TRAJ33 | TRAC | TRBV14 | — | TRBJ2-1 | TRBC2 |
| 20 | 644 | Ad5 | JAK2ep2 | TRAV13-1 | — | TRAJ43 | TRAC | TRBV11-2 | TRBD2 | TRBJ2-6 | TRBC2 |
| 21 | 118 | Ad5 | JAK2ep2 | TRAV19 | — | TRAJ17 | TRAC | TRBV5-6 | TRBD1 | TRBJ2-6 | TRBC2 |
| 22 | 118 | Endogenous | CALR | TRAV4 | — | TRAJ42 | TRAC | TRBV27 | TRBD1 | TRBJ2-2 | TRBC2 |
| 23 | LP263 | Exogenous | JAK2ep2 | TRAV14DV4 | — | TRAJ12 | TRAC | TRBV7-9 | TRBD1 | TRBJ1-6 | TRBC1 |
| 24 | 118 | Ad5 | JAK2ep2 | TRAV29DV5 | — | TRAJ42 | TRAC | TRBV2 | TRBD1 | TRBJ1-1 | TRBC1 |
| 25 | 274 | Ad5 | CALR | TRAV14DV4 | — | TRAJ22 | TRAC | TRBV2 | TRBD1 | TRBJ1-2 | TRBC1 |
| 26 | 118 | Ad5 | JAK2ep1 | TRAV29DV5 | — | TRAJ52 | TRAC | TRBV7-8 | — | TRBJ2-1 | TRBC2 |
| 27 | 118 | Ad5 | CALR | TRAV8-4 | — | TRAJ4 | TRAC | TRBV27 | TRBD1 | TRBJ1-5 | TRBC1 |
| 28 | 274 | Ad5 | CALR | TRAV19 | — | TRAJ30 | TRAC | TRBV20-1 | TRBD2 | TRBJ2-1 | TRBC2 |
| 29 | 118 | Ad5 | JAK2ep2 | TRAV29DV5 | — | TRAJ23 | TRAC | TRBV20-1 | TRBD1 | TRBJ2-1 | TRBC2 |
| 30 | 118 | Ad5 | CALR | TRAV9-2 | — | TRAJ20 | TRAC | TRBV20-1 | TRBD2 | TRBJ1-1 | TRBC1 |
| 31 | 274 | Ad5 | CALR | TRAV12-2 | — | TRAJ4 | TRAC | TRBV3-1 | TRBD2 | TRBJ2-7 | TRBC2 |
| 32 | 644 | Ad5 | JAK2ep2 | TRAV4 | — | TRAJ37 | TRAC | TRBV3-1 | TRBD2 | TRBJ2-7 | TRBC2 |
| 33 | 644 | Ad5 | JAK2ep2 | TRAV38- | — | TRAJ43 | TRAC | TRBV6-5 | TRBD1 | TRBJ2-7 | TRBC2 |
| | | | | 2DV8 | | | | | | | |
| 34 | 644 | Ad5 | JAK2ep2 | TRAV12-1 | — | TRAJ6 | TRAC | TRBV20-1 | — | TRBJ1-3 | TRBC1 |

Table 4 provides the full length alpha chain sequences as well as the nucleotide (NT) and amino acid (AA) sequences of the alpha chain CDR1, CDR2 and CDR3, defined by IMGT, for each of the thirty-four T-cell receptors described in Table 3. Table 5 provides the full length beta chain sequences as well as the nucleotide and amino acid sequences of the beta chain CDR1, CDR2 and CDR3, defined by IMGT, for each of the thirty-four T-cell receptors described in Table 3. For both Table 4 and Table 5, the TCR alpha or beta (full length) amino acid sequence that was derived from the scRNA analysis had an incomplete constant gene sequence. In order to complete this, alignments of the incomplete constant region sequence were performed against the canonical TCRB1 and TCRB2 sequences, and the additional missing portion appended. In two instances, the alignments had incomplete query coverage, in these cases the overhanging portion in the incomplete gene was deleted and the correct TCRB sequence appended. For the TCR alpha gene, the missing portion of the TCR alpha gene sequence was appended. The full constant gene is identified by name in Table 3. The VJ/VDJ portion of the TCR alpha or beta (full length) amino acid sequence is underlined, the constant region is in underlined and bold, and the unmarked sequence is the leader sequence. For the corresponding nucleotide sequences in Tables 4 and 5, the leader sequence is underlined, the constant region is underlined and bolded, and the VJ/VDJ portion of the full length TCR nucleotide sequence is unmarked.

TABLE 4

| | TCR ALPHA | TCR ALPHA | TCR ALPHA | TCR ALPHA | | | | | | |
|---|---|---|---|---|---|---|---|---|---|---|
| | (FULL LENGTH) | (FULL | (V-J | (V-J | CDR1 | CDR1 | CDR2 | CDR2 | CDR3 | CDR3 |
| ID | (NT) | LENGTH) (AA) | REGION) (NT) | REGION) (AA) | (NT) | (AA) | (NT) | (AA) | (NT) | (AA) |
| 1 | GGGGCAGGATGTGATTCTAA | MKKLLAMILW | GAGCTGAAAG | ELKVEQNPLFL | ACCA | TTSDR | ACCA | LLSNG | GCCG | CAAR |
| | TTGGTTGGAACATCTTTTGA | LQLDRLSGELK | TGGAACAAAA | SMQEGKNYTIY | CTTC | (SEQ | CTTC | AV | CCCG | SYNT |
| | AATCGTGTTTCTGTAGAGAA | VEQNPLFLSMQ | CCCTCTGTTCC | CNYSTTSDRLY | AGAC | ID NO: | AGAC | (SEQ | ATCTT | DKLIF |
| | AGAAAAACTACCATATTTGG | EGKNYTIYCNY | TGAGCATGCA | WYRQDPGKSLE | AGA | 3) | AGA | ID NO: | ATAA | (SEQ |
| | ATAGCCCTGGCCAACTTTCA | STTSDRLYWYR | GGAGGGAAAA | SLFVLLSNGAV | (SEQ | | (SEQ | 5) | CACC | ID NO: |
| | AGGCTCCTAAATCTGAGTTT | QDPGKSLESLF | AACTATACCAT | KQEGRLMASLD | ID NO: | | ID NO: | | GACA | 7) |
| | TCAGTGAACTGGACAGAAAA | VLLSNGAVKQE | CTACTGCAATT | TKARLSTLHITA | 2) | | 4) | | AGCT | |
| | AAAAAATGAAGAAGCTACTA | GRLMASLDTKA | ATTCAACCACT | AVHDLSATYFC | | | | | CATC | |
| | GCAATGATTCTGTGGCTTCA | RLSTLHITAAV | TCAGACAGACT | AARSYNTDKLI | | | | | (SEQ | |
| | ACTAGACCGGTTAAGTGGAG | HDLSATYFCAA | GTATTGGTACA | FGTGTRLQVFP | | | | | ID NO: | |
| | AGCTGAAAGTGGAACAAAA | RSYNTDKLIFGT | GGCAGGATCCT | (SEQ ID NO: | | | | | 6) | |
| | CCCTCTGTTCCTGAGCATGC | GTRLQVFPNIQ | GGGAAAAGTC | 479) | | | | | | |
| | AGGAGGGAAAAAACTATAC | NPDPAVYQLR | TGGAATCTCTG | | | | | | | |
| | CATCTACTGCAATTATTCAA | DSKSSDKSVCL | TTTGTGTTGCT | | | | | | | |
| | CCACTTCAGACAGACTGTAT | FTDFDSQTNVS | ATCAAATGGA | | | | | | | |
| | TGGTACAGGCAGGATCCTGG | QSKDSDVYITD | GCAGTGAAGC | | | | | | | |
| | GAAAAGTCTGGAATCTCTGT | KTVLDMRSMD | AGGAGGGACG | | | | | | | |
| | TTGTGTTGCTATCAAATGGA | FKSNSAVAWS | ATTAATGGCCT | | | | | | | |
| | GCAGTGAAGCAGGAGGGAC | NKSDFACANA | CACTTGATACC | | | | | | | |
| | GATTAATGGCCTCACTTGAT | FNNSIIPEDTFF | AAAGCCCGTCT | | | | | | | |
| | ACCAAAGCCCGTCTCAGCAC | PSPESSCDVKL | CAGCACCCTCC | | | | | | | |
| | CCTCCACATCACAGCTGCCG | VEKSFETDTNL | ACATCACAGCT | | | | | | | |
| | TGCATGACCTCTCTGCCACCT | NFQNLSVIGFR | GCCGTGCATGA | | | | | | | |
| | ACTTCTGTGCCGCCCGATCTT | ILLLKVAGFNL | CCTCTCTGCCA | | | | | | | |
| | ATAACACCGACAAGCTCATC | LMTLRLWSS | CCTACTTCTGT | | | | | | | |
| | TTTGGGACTGGGACCAGATT | (SEQ ID NO: | GCCGCCCGATC | | | | | | | |
| | ACAAGTCTTTCCAAATATCC | 477) | TTATAACACCG | | | | | | | |
| | AGAACCCTGACCCTGCCGT | | ACAAGCTCATC | | | | | | | |
| | GTACCAGCTGAGAGACTCT | | TTTGGGACTGG | | | | | | | |
| | AAATCCAGTGACAAGTCTG | | GACCAGATTAC | | | | | | | |
| | TCTGCCTATTCACCGATTT | | AAGTCTTTCCA | | | | | | | |
| | TGATTCTCAAACAAATGTG | | A | | | | | | | |
| | TCACAAAGTAAGGATTCTG | | (SEQ ID NO: | | | | | | | |
| | ATGTGTATATCACAGACAA | | 478) | | | | | | | |
| | AACTGTGCTAGACATGAGG | | | | | | | | | |
| | TCTATGGACTTCAAGAGCA | | | | | | | | | |
| | ACAGTGCTGTGGCCTGGAG | | | | | | | | | |
| | CAACAAATCTGACTTTGCA | | | | | | | | | |
| | TGTGCAAACGCCTTCAACA | | | | | | | | | |
| | ACAGCATTATTCCAGAAGA | | | | | | | | | |
| | CACCTTCTTCCCCAGCCCA | | | | | | | | | |
| | GAAAGTTCCTGTGATGTCA | | | | | | | | | |
| | AGCTGGTCGAGAAAAGCTT | | | | | | | | | |
| | TGAAACAGATACGAACCTA | | | | | | | | | |
| | AACTTTCAAAACCTGTCAG | | | | | | | | | |
| | TGATTGGGTTCCGAATCCT | | | | | | | | | |
| | CCTCCTGAAAGTGGCCGGG | | | | | | | | | |
| | TTTAATCTGCTCATGACGC | | | | | | | | | |
| | TGCGGCTGTGGTCCAGC | | | | | | | | | |
| | (SEQ ID NO: 1) | | | | | | | | | |
| 2 | TGGGGAGTCTTGCTCCTCAC | MASAPISMLAM | GCTCAGTCAGT | AQSVAQPEDQV | GTCT | VSGN | TACA | YITGD | GCTG | CAVR |
| | AGAGCTTTGAGGAGCTGGAT | LFTLSGLRAQS | GGCTCAGCCG | NVAEGNPLTVK | CTGG | PY | TCAC | NLV | TGAG | DMGG |
| | CAAAATTGTGCTCCACAGAG | VAQPEDQVNV | GAAGATCAGG | CTYSVSGNPYL | AAAC | (SEQ | AGGG | (SEQ | AGAC | SARQ |
| | AGAAGATACCGGTGTCGGGA | AEGNPLTVKCT | TCAACGTTGCT | FWYVQYPNRG | CCTT | ID NO: | GATA | ID NO: | ATGG | LTF |
| | AGCACCAGTGCCCTGAGGAA | YSVSGNPYLFW | GAAGGGAATC | LQFLLKYITGD | AT | 10) | ACCT | 12) | GGGG | (SEQ |
| | GGGCCATTTCCAAAAGCCCT | YVQYPNRGLQF | CTCTGACTGTG | NLVKGSYGFEA | (SEQ | | GGTT | | TTCTG | ID NO: |
| | GTGCTGACACAGGGTTGCTG | LLKYITGDNLV | AAATGCACCTA | EFNKSQTSFHL | ID NO: | | (SEQ | | CAAG | 14) |
| | GTTCCTCTTCAAGAGCCCAC | KGSYGFEAEFN | TTCAGTCTCTG | KKPSALVSDSA | 9) | | ID NO: | | GCAA | |
| | TCTCTGGGGTGGGGCCATAT | KSQTSFHLKKP | GAAACCCTTAT | LYFCAVRDMG | | | 11) | | CTGA | |
| | CTCCAGCAGAGGTGGGCTGG | SALVSDSALYF | CTTTTTTGGTA | GSARQLTFGSG | | | | | CC | |
| | AAAGGACCCCCCCAATCCCG | CAVRDMGGSA | TGTTCAATACC | TQLTVLP | | | | | (SEQ | |
| | CCCGCCGTGAGCTTAGCTGG | RQLTFGSGTQL | CCAACCGAGG | (SEQ ID NO: | | | | | ID NO: | |
| | AGCCATGGCCTCTGCACCCA | TVLPDIQNPDP | CCTCCAGTTCC | 482) | | | | | 13) | |
| | TCTCGATGCTTGCGATGCTCT | AVYQLRDSKSS | TTCTGAAATAC | | | | | | | |
| | TCACATTGAGTGGGCTGAGA | DKSVCLFTDFD | ATCACAGGGG | | | | | | | |
| | GCTCAGTCAGTGGCTCAGCC | SQTNVSQSKDS | ATAACCTGGTT | | | | | | | |
| | GGAAGATCAGGTCAACGTTG | DVYITDKTVLD | AAAGGCAGCT | | | | | | | |
| | CTGAAGGGAATCCTCTGACT | MRSMDFKSNS | ATGGCTTTGAA | | | | | | | |
| | GTGAAATGCACCTATTCAGT | AVAWSNKSDF | GCTGAATTTAA | | | | | | | |
| | CTCTGGAAACCCTTATCTTTT | ACANAFNNSII | CAAGAGCCAA | | | | | | | |
| | TTGGTATGTTCAATACCCCA | PEDTFFPSPESS | ACCTCCTTCCA | | | | | | | |
| | ACCGAGGCCTCCAGTTCCTT | CDVKLVEKSF | CCTGAAGAAA | | | | | | | |
| | CTGAAATACATCACAGGGGA | ETDTNLNFQN | CCATCTGCCCT | | | | | | | |
| | TAACCTGGTTAAAGGCAGCT | LSVIGFRILLL | TGTGAGCGACT | | | | | | | |
| | ATGGCTTTGAAGCTGAATTT | KVAGFNLLMT | CCGCTTTGTAC | | | | | | | |
| | AACAAGAGCCAAACCTCCTT | LRLWSS | TTCTGTGCTGT | | | | | | | |
| | CCACCTGAAGAAACCATCTG | (SEQ ID NO: | GAGAGACATG | | | | | | | |
| | CCCTTGTGAGCGACTCCGCT | 480) | GGGGGTTCTGC | | | | | | | |
| | TTGTACTTCTGTGCTGTGAGA | | AAGGCAACTG | | | | | | | |
| | GACATGGGGGGTTCTGCAAG | | ACCTTTGGATC | | | | | | | |
| | GCAACTGACCTTTGGATCTG | | TGGGACACAA | | | | | | | |
| | GGACACAATTGACTGTTTTA | | TTGACTGTTTT | | | | | | | |
| | CCTGATATCCAGAACCCTG | | ACCTG | | | | | | | |
| | ACCCTGCCGTGTACCAGCT | | (SEQ ID NO: | | | | | | | |
| | GAGAGACTCTAAATCCAGT | | 481) | | | | | | | |
| | GACAAGTCTGTCTGCCTAT | | | | | | | | | |
| | TCACCGATTTTGATTCTCA | | | | | | | | | |
| | AACAAATGTGTCACAAAGT | | | | | | | | | |
| | AAGGATTCTGATGTGTATA | | | | | | | | | |
| | TCACAGACAAAACTGTGCT | | | | | | | | | |
| | AGACATGAGGTCTATGGAC | | | | | | | | | |
| | TTCAAGAGCAACAGTGCTG | | | | | | | | | |
| | TGGCCTGGAGCAACAAATC | | | | | | | | | |
| | TGACTTTGCATGTGCAAAC | | | | | | | | | |
| | GCCTTCAACAACAGCATTA | | | | | | | | | |
| | TTCCAGAAGACACCTTCTT | | | | | | | | | |
| | CCCCAGCCCAGAAAGTTCC | | | | | | | | | |
| | TGTGATGTCAAGCTGGTCG | | | | | | | | | |
| | AGAAAAGCTTTGAAACAGA | | | | | | | | | |
| | TACGAACCTAAACTTTCAA | | | | | | | | | |
| | AACCTGTCAGTGATTGGGT | | | | | | | | | |
| | TCCGAATCCTCCTCCTGAA | | | | | | | | | |
| | AGTGGCCGGGTTTAATCTG | | | | | | | | | |
| | CTCATGACGCTGCGGCTGT | | | | | | | | | |
| | GGTCCAGC | | | | | | | | | |
| | (SEQ ID NO: 8) | | | | | | | | | |
| 3 | GAGCCTGAGTGACAGCTGCT | NMRQVARVIVF | CTTGCTAAGAC | LAKTTQPISMD | AACA | NIATN | GGAT | GYKT | CTCG | CLVG |
| | GGTGTGGGCCCTGGCAGTTG | LTLSTLSLAKTT | CACCCAGCCCA | SYEGQEVNITCS | TTGCT | DY | ACAA | K | TGGG | TFSGS |
| | CTGCTGGGCTCATTGCAGCT | QPISMDSYEGQ | TCTCCATGGAC | HNNIATNDYIT | ACAA | (SEQ | GACA | (SEQ | GACG | ARQL |
| | CAGACACAGCAAAAGAGCCT | EVNITCSHNNIA | TCATATGAAGG | WYQQFPSQGPR | ATGA | ID NO: | AAA | ID NO: | TTTTC | TF |
| | AGAACCTGGGTCCTAGTTTG | TNDYITWYQQF | ACAAGAAGTG | FIIQGYKTKVTN | TTAT | 17) | (SEQ | 19) | TGGT | (SEQ |
| | CACCTAGAATATGAGGCAAG | PSQGPRFIIQGY | AACATAACCTG | EVASLFIPADRK | (SEQ | | ID NO: | | TCTG | ID NO: |
| | TGGCGAGAGTGATCGTGTTC | KTKVTNEVASL | TAGCCACAAC | SSTLSLPRVSLS | ID NO: | | 18) | | CAAG | 21) |
| | CTGACCCTGAGTACTTTGAG | FIPADRKSSTLS | AACATTGCTAC | DTAVYYCLVGT | 16) | | | | GCAA | |
| | CCTTGCTAAGACCACCCAGC | LPRVSLSDTAV | AAATGATTATA | FSGSARQLTFGS | | | | | CTGA | |
| | CCATCTCCATGGACTCATAT | YYCLVGTFSGS | TCACGTGGTAC | GTQLTVLP | | | | | CC | |
| | GAAGGACAAGAAGTGAACA | ARQLTFGSGTQ | CAACAGTTTCC | (SEQ ID NO: | | | | | (SEQ | |
| | TAACCTGTAGCCACAACAAC | LTVLPDIQNPD | CAGCCAAGGA | 485) | | | | | ID NO: | |
| | ATTGCTACAAATGATTATAT | PAVYQLRDSKS | CCACGATTTAT | | | | | | 20) | |
| | CACGTGGTACCAACAGTTTC | SDKSVCLFTDF | TATTCAAGGAT | | | | | | | |
| | CCAGCCAAGGACCACGATTT | DSQTNVSQSKD | ACAAGACAAA | | | | | | | |
| | ATTATTCAAGGATACAAGAC | SDVYITDKTVL | AGTTACAAAC | | | | | | | |
| | AAAAGTTACAAACGAAGTGG | DMRSMDFKSN | GAAGTGGCCTC | | | | | | | |
| | CCTCCCTGTTTATCCCTGCCG | SAVAWSNKSD | CCTGTTTATCC | | | | | | | |
| | ACAGAAAGTCCAGCACTCTG | FACANAFNNSI | CTGCCGACAG | | | | | | | |
| | AGCCTGCCCCGGGTTTCCCT | IPEDTFFPSPES | AAAGTCCAGC | | | | | | | |
| | GAGCGACACTGCTGTGTACT | SCDVKLVEKSF | ACTCTGAGCCT | | | | | | | |
| | ACTGCCTCGTGGGGACGTTT | ETDTNLNFQN | GCCCCGGGTTT | | | | | | | |
| | TCTGGTTCTGCAAGGCAACT | LSVIGFRILLL | CCCTGAGCGAC | | | | | | | |
| | GACCTTTGGATCTGGGACAC | KVAGFNLLMT | ACTGCTGTGTA | | | | | | | |
| | AATTGACTGTTTTACCTGATA | LRLWSS | CTACTGCCTCG | | | | | | | |
| | TCCAGAACCCTGACCCTGC | (SEQ ID NO: | TGGGGACGTTT | | | | | | | |
| | CGTGTACCAGCTGAGAGAC | 483) | TCTGGTTCTGC | | | | | | | |
| | TCTAAATCCAGTGACAAGT | | AAGGCAACTG | | | | | | | |
| | CTGTCTGCCTATTCACCGA | | ACCTTTGGATC | | | | | | | |
| | TTTTGATTCTCAAACAAAT | | TGGGACACAA | | | | | | | |
| | GTGTCACAAAGTAAGGATT | | TTGACTGTTTT | | | | | | | |
| | CTGATGTGTATATCACAGA | | ACCTG (SEQ ID | | | | | | | |
| | CAAAACTGTGCTAGACATG | | NO: 484) | | | | | | | |
| | AGGTCTATGGACTTCAAGA | | | | | | | | | |
| | GCAACAGTGCTGTGGCCTG | | | | | | | | | |
| | GAGCAACAAATCTGACTTT | | | | | | | | | |
| | GCATGTGCAAACGCCTTCA | | | | | | | | | |
| | ACAACAGCATTATTCCAGA | | | | | | | | | |
| | AGACACCTTCTTCCCCAGC | | | | | | | | | |
| | CCAGAAAGTTCCTGTGATG | | | | | | | | | |
| | TCAAGCTGGTCGAGAAAAG | | | | | | | | | |
| | CTTTGAAACAGATACGAAC | | | | | | | | | |
| | CTAAACTTTCAAAACCTGT | | | | | | | | | |
| | CAGTGATTGGGTTCCGAAT | | | | | | | | | |
| | CCTCCTCCTGAAAGTGGCC | | | | | | | | | |
| | GGGTTTAATCTGCTCATGA | | | | | | | | | |
| | CGCTGCGGCTGTGGTCCAG | | | | | | | | | |
| | C (SEQ ID NO: 15) | | | | | | | | | |
| 4 | GGCATAATAAATGTCTACGC | MVKIRQFLLAIL | AAAAATGAAG | KNEVEQSPQNL | GTAG | VGISA | CTGA | LSSGK | GCTG | CAVS |
| | CTCATGCCACTAGGTGGCAA | WLQLSCVSAAK | TGGAGCAGAG | TAQEGEFITINC | GAAT | (SEQ | GCTC | (SEQ | TCAG | TSMY |
| | TGTGGGTGTTATACTGAAAA | NEVEQSPQNLT | TCCTCAGAACC | SYSVGISALHW | AAGT | ID NO: | AGGG | ID NO: | CACC | SGGG |
| | GATCACAGATGGTTCACTTT | AQEGEFITINCS | TGACTGCCCAG | LQQHPGGGIVS | GCC | 24) | AAG | 26) | TCTAT | ADGL |
| | GCAAGTAAAACTGTAAATGT | YSVGISALHWL | GAAGGAGAAT | LFMLSSGKKKH | (SEQ | | (SEQ | | GTAT | TF |
| | TCTTAAGTGTGCATTTCTGCT | QQHPGGGIVSL | TTATCACAATC | GRLIATINIQEK | ID NO: | | ID NO: | | TCAG | (SEQ |
| | GCTTCTGATGGGCTGAAAAT | FMLSSGKKKHG | AACTGCAGTTA | HSSLHITASHPR | 23) | | 25) | | GAGG | ID NO: |
| | CCCCTTTGATTTCTAAAGTAA | RLIATINIQEKH | CTCGGTAGGA | DSAVYICAVST | | | | | AGGT | 28) |
| | ATGTAGAGACGTTTTAAAAA | SSLHITASHPRD | ATAAGTGCCTT | SMYSGGGADG | | | | | GCTG | |
| | TAAAGGACTCCTTTGTCCAA | SAVYICAVSTS | ACACTGGCTGC | LTFGKGTHLIIQ | | | | | ACGG | |
| | GATATATTCCGAAATCCTCC | MYSGGGADGL | AACAGCATCC | P | | | | | ACTC | |
| | AACAGAGACCTGTGTGAGCT | TFGKGTHLIIQP | AGGAGGAGGC | (SEQ ID NO: | | | | | ACC | |
| | TCTGCTGCAGTAATAATGGT | YIQNPDPAVYQ | ATTGTTTCCTT | 488) | | | | | (SEQ | |
| | GAAGATCCGGCAATTTTTGT | LRDSKSSDKSV | GTTTATGCTGA | | | | | | ID NO: | |
| | TGGCTATTTTGTGGCTTCAGC | CLFTDFDSQTN | GCTCAGGGAA | | | | | | 27) | |
| | TAAGCTGTGTAAGTGCCGCC | VSQSKDSDVYI | GAAGAAGCAT | | | | | | | |
| | AAAAATGAAGTGGAGCAGA | TDKTVLDMRS | GGAAGATTAA | | | | | | | |
| | GTCCTCAGAACCTGACTGCC | MDFKSNSAVA | TTGCCACAATA | | | | | | | |
| | CAGGAAGGAGAATTTATCAC | WSNKSDFACA | AACATACAGG | | | | | | | |
| | AATCAACTGCAGTTACTCGG | NAFNNSIIPEDT | AAAAGCACAG | | | | | | | |
| | TAGGAATAAGTGCCTTACAC | FFPSPESSCDV | CTCCCTGCACA | | | | | | | |
| | TGGCTGCAACAGCATCCAGG | KLVEKSFETDT | TCACAGCCTCC | | | | | | | |
| | AGGAGGCATTGTTTCCTTGTT | NLNFQNLSVIG | CATCCCAGAG | | | | | | | |
| | TATGCTGAGCTCAGGGAAGA | FRILLLKVAGF | ACTCTGCCGTC | | | | | | | |
| | AGAAGCATGGAAGATTAATT | NLLMTLRLWS | TACATCTGTGC | | | | | | | |
| | GCCACAATAAACATACAGGA | S | TGTCAGCACCT | | | | | | | |
| | AAAGCACAGCTCCCTGCACA | (SEQ ID NO: | CTATGTATTCA | | | | | | | |
| | TCACAGCCTCCCATCCCAGA | 486) | GGAGGAGGTG | | | | | | | |
| | GACTCTGCCGTCTACATCTGT | | CTGACGGACTC | | | | | | | |
| | GCTGTCAGCACCTCTATGTA | | ACCTTTGGCAA | | | | | | | |
| | TTCAGGAGGAGGTGCTGACG | | AGGGACTCATC | | | | | | | |
| | GACTCACCTTTGGCAAAGGG | | TAATCATCCAG | | | | | | | |
| | ACTCATCTAATCATCCAGCC | | CCCT | | | | | | | |
| | CTATATCCAGAACCCTGAC | | (SEQ ID NO: | | | | | | | |
| | CCTGCCGTGTACCAGCTGA | | 487) | | | | | | | |
| | GAGACTCTAAATCCAGTGA | | | | | | | | | |
| | CAAGTCTGTCTGCCTATTC | | | | | | | | | |
| | ACCGATTTTGATTCTCAAA | | | | | | | | | |
| | CAAATGTGTCACAAAGTAA | | | | | | | | | |
| | GGATTCTGATGTGTATATC | | | | | | | | | |
| | ACAGACAAAACTGTGCTAG | | | | | | | | | |
| | ACATGAGGTCTATGGACTT | | | | | | | | | |
| | CAAGAGCAACAGTGCTGTG | | | | | | | | | |
| | GCCTGGAGCAACAAATCTG | | | | | | | | | |
| | ACTTTGCATGTGCAAACGC | | | | | | | | | |
| | CTTCAACAACAGCATTATT | | | | | | | | | |
| | CCAGAAGACACCTTCTTCC | | | | | | | | | |
| | CCAGCCCAGAAAGTTCCTG | | | | | | | | | |
| | TGATGTCAAGCTGGTCGAG | | | | | | | | | |
| | AAAAGCTTTGAAACAGATA | | | | | | | | | |
| | CGAACCTAAACTTTCAAAA | | | | | | | | | |
| | CCTGTCAGTGATTGGGTTC | | | | | | | | | |
| | CGAATCCTCCTCCTGAAAG | | | | | | | | | |
| | TGGCCGGGTTTAATCTGCT | | | | | | | | | |
| | CATGACGCTGCGGCTGTGG | | | | | | | | | |
| | TCCAGC | | | | | | | | | |
| | (SEQ ID NO: 22) | | | | | | | | | |
| 5 | GGCTTAGTGGTGAATTAGGG | MMKSLRVLLVI | CAGAAGGAGG | QKEVEQNSGPL | GACC | DRGS | ATAT | IYSNG | GCCG | CAVK |
| | GTGTTAAAAAGAGCATCATT | LWLQLSWVWS | TGGAGCAGAA | SVPEGAIASLNC | GAGG | QS | ACTC | D | TGAA | GGYA |
| | TTTTTGAACTGGTAAAGCAG | QQKEVEQNSGP | TTCTGGACCCC | TYSDRGSQSFF | TTCCC | (SEQ | CAAT | (SEQ | GGGA | LNF |
| | ATTCTTTTTATGATTTTTAAA | LSVPEGAIASLN | TCAGTGTTCCA | WYRQYSGKSPE | AGTC | ID NO: | GGTG | ID NO: | GGGT | (SEQ |
| | GTAGAAATATCCATTCCAGG | CTYSDRGSQSF | GAGGGAGCCA | LIMSIYSNGDKE | C | 31) | AC | 33) | ATGC | ID NO: |
| | TGCATTTTTTAAGGGTTTAAA | FWYRQYSGKSP | TTGCCTCTCTC | DGRFTAQLNKA | (SEQ | | (SEQ | | ACTC | 35) |
| | ATTTGAATCCTCAGTGAACC | ELIMSIYSNGDK | AACTGCACTTA | SQYVSLLIRDSQ | ID NO: | | ID NO: | | AAC | |
| | AGGGCAGAGAAGAATGATG | EDGRFTAQLNK | CAGTGACCGA | PSDSATYLCAV | 30) | | 32) | | (SEQ | |
| | AAATCCTTGAGAGTTTTACT | ASQYVSLLIRDS | GGTTCCCAGTC | KGGYALNFGK | | | | | ID NO: | |
| | AGTGATCCTGTGGCTTCAGT | QPSDSATYLCA | CTTCTTCTGGT | GTSLLVTP | | | | | 34) | |
| | TGAGCTGGGTTTGGAGCCAA | VKGGYALNFG | ACAGACAATA | (SEQ ID NO: | | | | | | |
| | CAGAAGGAGGTGGAGCAGA | KGTSLLVTPHI | TTCTGGGAAAA | 491) | | | | | | |
| | ATTCTGGACCCCTCAGTGTTC | QNPDPAVYQL | GCCCTGAGTTG | | | | | | | |
| | CAGAGGGAGCCATTGCCTCT | RDSKSSDKSVC | ATAATGTCCAT | | | | | | | |
| | CTCAACTGCACTTACAGTGA | LFTDFDSQTNV | ATACTCCAATG | | | | | | | |
| | CCGAGGTTCCCAGTCCTTCTT | SQSKDSDVYIT | GTGACAAAGA | | | | | | | |
| | CTGGTACAGACAATATTCTG | DKTVLDMRSM | AGATGGAAGG | | | | | | | |
| | GGAAAAGCCCTGAGTTGATA | DFKSNSAVAW | TTTACAGCACA | | | | | | | |
| | ATGTCCATATACTCCAATGG | SNKSDFACANA | GCTCAATAAA | | | | | | | |
| | TGACAAAGAAGATGGAAGG | FNNSIIPEDTFF | GCCAGCCAGT | | | | | | | |
| | TTTACAGCACAGCTCAATAA | PSPESSCDVKL | ATGTTTCTCTG | | | | | | | |
| | AGCCAGCCAGTATGTTTCTC | VEKSFETDTNL | CTCATCAGAGA | | | | | | | |
| | TGCTCATCAGAGACTCCCAG | NFQNLSVIGFR | CTCCCAGCCCA | | | | | | | |
| | CCCAGTGATTCAGCCACCTA | ILLLKVAGFNL | GTGATTCAGCC | | | | | | | |
| | CCTCTGTGCCGTGAAGGGAG | LMTLRLWSS | ACCTACCTCTG | | | | | | | |
| | GGTATGCACTCAACTTCGGC | (SEQ ID NO: | TGCCGTGAAG | | | | | | | |
| | AAAGGCACCTCGCTGTTGGT | 489) | GGAGGGTATG | | | | | | | |
| | CACACCCCATATCCAGAACC | | CACTCAACTTC | | | | | | | |
| | CTGACCCTGCCGTGTACCA | | GGCAAAGGCA | | | | | | | |
| | GCTGAGAGACTCTAAATCC | | CCTCGCTGTTG | | | | | | | |
| | AGTGACAAGTCTGTCTGCC | | GTCACACCCC | | | | | | | |
| | TATTCACCGATTTTGATTCT | | (SEQ ID NO: | | | | | | | |
| | CAAACAAATGTGTCACAAA | | 490) | | | | | | | |
| | GTAAGGATTCTGATGTGTA | | | | | | | | | |
| | TATCACAGACAAAACTGTG | | | | | | | | | |
| | CTAGACATGAGGTCTATGG | | | | | | | | | |
| | ACTTCAAGAGCAACAGTGC | | | | | | | | | |
| | TGTGGCCTGGAGCAACAAA | | | | | | | | | |
| | TCTGACTTTGCATGTGCAA | | | | | | | | | |
| | ACGCCTTCAACAACAGCAT | | | | | | | | | |
| | TATTCCAGAAGACACCTTC | | | | | | | | | |
| | TTCCCCAGCCCAGAAAGTT | | | | | | | | | |
| | CCTGTGATGTCAAGCTGGT | | | | | | | | | |
| | CGAGAAAAGCTTTGAAACA | | | | | | | | | |
| | GATACGAACCTAAACTTTC | | | | | | | | | |
| | AAAACCTGTCAGTGATTGG | | | | | | | | | |
| | GTTCCGAATCCTCCTCCTG | | | | | | | | | |
| | AAAGTGGCCGGGTTTAATC | | | | | | | | | |
| | TGCTCATGACGCTGCGGCT | | | | | | | | | |
| | GTGGTCCAGC | | | | | | | | | |
| | (SEQ ID NO: 29) | | | | | | | | | |
| 6 | GGCCCATTTCCTGTTCTGAA | MACPGFLWAL | GCTCAGACAGT | AQTVTQSQPEM | ACCA | TSESD | CAAG | QEAY | GCTT | CAYSP |
| | GCAGCTACGGCACCAGTGCA | VISTCLEFSMAQ | CACTCAGTCTC | SVQEAETVTLS | GTGA | YY | AAGC | KQQN | ATAG | PLPGN |
| | GCTGATACTCAAGGTTCAGA | TVTQSQPEMSV | AACCAGAGAT | CTYDTSESDYY | GAGT | (SEQ | TTAT | (SEQ | TCCC | QFYF |
| | TCAGAAGAGGAGGCTTCTCA | QEAETVTLSCT | GTCTGTGCAGG | LFWYKQPPSRQ | GATT | ID NO: | AAGC | ID NQ | CCCC | (SEQ |
| | CCCTGCAGCAGGGACCTGTG | YDTSESDYYLF | AGGCAGAGAC | MILVIRQEAYK | ATTA | 38) | AACA | 40) | TCCC | ID NO: |
| | AGCATGGCATGCCCTGGCTT | WYKQPPSRQMI | CGTGACCCTGA | QQNATENRFSV | T | | GAAT | | CGGT | 42) |
| | CCTGTGGGCACTTGTGATCT | LVIRQEAYKQQ | GCTGCACATAT | NFQKAAKSFSL | (SEQ | | (SEQ | | AACC | |
| | CCACCTGTCTTGAATTTAGC | NATENRFSVNF | GACACCAGTG | KISDSQLGDAA | ID NO: | | ID NO: | | AGTT | |
| | ATGGCTCAGACAGTCACTCA | QKAAKSFSLKIS | AGAGTGATTAT | MYFCAYSPPLP | 37) | | 39) | | CTAT | |
| | GTCTCAACCAGAGATGTCTG | DSQLGDAAMY | TATTTATTCTG | GNQFYFGTGTS | | | | | (SEQ | |
| | TGCAGGAGGCAGAGACCGTG | FCAYSPPLPGN | GTACAAGCAG | LTVIP | | | | | ID NO: | |
| | ACCCTGAGCTGCACATATGA | QFYFGTGTSLT | CCTCCCAGCAG | (SEQ ID NO: | | | | | 41) | |
| | CACCAGTGAGAGTGATTATT | VIPNIQNPDPA | GCAGATGATTC | 494) | | | | | | |
| | ATTTATTCTGGTACAAGCAG | VYQLRDSKSSD | TCGTTATTCGC | | | | | | | |
| | CCTCCCAGCAGGCAGATGAT | KSVCLFTDFDS | CAAGAAGCTT | | | | | | | |
| | TCTCGTTATTCGCCAAGAAG | QTNVSQSKDSD | ATAAGCAACA | | | | | | | |
| | CTTATAAGCAACAGAATGCA | VYITDKTVLD | GAATGCAACA | | | | | | | |
| | ACAGAGAATCGTTTCTCTGT | MRSMDFKSNS | GAGAATCGTTT | | | | | | | |
| | GAACTTCCAGAAAGCAGCCA | AVAWSNKSDF | CTCTGTGAACT | | | | | | | |
| | AATCCTTCAGTCTCAAGATC | ACANAFNNSII | TCCAGAAAGC | | | | | | | |
| | TCAGACTCACAGCTGGGGGA | PEDTFFPSPESS | AGCCAAATCCT | | | | | | | |
| | TGCCGCGATGTATTTCTGTGC | CDVKLVEKSF | TCAGTCTCAAG | | | | | | | |
| | TTATAGTCCCCCCCTCCCCGG | ETDTNLNFQN | ATCTCAGACTC | | | | | | | |
| | TAACCAGTTCTATTTTGGGA | LSVIGFRILLL | ACAGCTGGGG | | | | | | | |
| | CAGGGACAAGTTTGACGGTC | KVAGFNLLMT | GATGCCGCGAT | | | | | | | |
| | ATTCCAAATATCCAGAACCC | LRLWSS | GTATTTCTGTG | | | | | | | |
| | TGACCCTGCCGTGTACCAG | (SEQ ID NO: | CTTATAGTCCC | | | | | | | |
| | CTGAGAGACTCTAAATCCA | 492) | CCCCTCCCCGG | | | | | | | |
| | GTGACAAGTCTGTCTGCCT | | TAACCAGTTCT | | | | | | | |
| | ATTCACCGATTTTGATTCT | | ATTTTGGGACA | | | | | | | |
| | CAAACAAATGTGTCACAAA | | GGGACAAGTTT | | | | | | | |
| | GTAAGGATTCTGATGTGTA | | GACGGTCATTC | | | | | | | |
| | TATCACAGACAAAACTGTG | | CAA | | | | | | | |
| | CTAGACATGAGGTCTATGG | | (SEQ ID NO: | | | | | | | |
| | ACTTCAAGAGCAACAGTGC | | 493) | | | | | | | |
| | TGTGGCCTGGAGCAACAAA | | | | | | | | | |
| | TCTGACTTTGCATGTGCAA | | | | | | | | | |
| | ACGCCTTCAACAACAGCAT | | | | | | | | | |
| | TATTCCAGAAGACACCTTC | | | | | | | | | |
| | TTCCCCAGCCCAGAAAGTT | | | | | | | | | |
| | CCTGTGATGTCAAGCTGGT | | | | | | | | | |
| | CGAGAAAAGCTTTGAAACA | | | | | | | | | |
| | GATACGAACCTAAACTTTC | | | | | | | | | |
| | AAAACCTGTCAGTGATTGG | | | | | | | | | |
| | GTTCCGAATCCTCCTCCTG | | | | | | | | | |
| | AAAGTGGCCGGGTTTAATC | | | | | | | | | |
| | TGCTCATGACGCTGCGGCT | | | | | | | | | |
| | GTGGTCCAGC | | | | | | | | | |
| | (SEQ ID NO: 36) | | | | | | | | | |
| 7 | GCTTTTTTGAAATCGTGTTTC | MKKLLAMILW | GAGCTGAAAG | ELKVEQNPLFL | ACCA | TTSDR | TTGCT | LLSNG | GCCG | CAGE |
| | TGTAGAGAAAGAAAAACTAC | LQLDRLSGELK | TGGAACAAAA | SMQEGKNYTIY | CTTC | (SEQ | ATCA | AV | GTGA | PDSN |
| | CATATTTGGATAGCCCTGGC | VEQNPLFLSMQ | CCCTCTGTTCC | CNYSTTSDRLY | AGAC | ID NO: | AATG | (SEQ | ACCT | YQLI |
| | CAACTTTCAAGGCTCCTAAA | EGKNYTIYCNY | TGAGCATGCA | WYRQDPGKSLE | AGA | 45) | GAGC | ID NO: | GATA | W |
| | TCTGAGTTTTCAGTGAACTG | STTSDRLYWYR | GGAGGGAAAA | SLFVLLSNGAV | (SEQ | | AGTG | 47) | GCAA | (SEQ |
| | GACAGAAAAAAAAAATGAA | QDPGKSLESLF | AACTATACCAT | KQEGRLMASLD | ID NO: | | (SEQ | | CTAT | ID NO: |
| | GAAGCTACTAGCAATGATTC | VLLSNGAVKQE | CTACTGCAATT | TKARLSTLHITA | 44) | | ID NO: | | CAGT | 49) |
| | TGTGGCTTCAACTAGACCGG | GRLMASLDTKA | ATTCAACCACT | AVHDLSATYFC | | | 46) | | TAAT | |
| | TTAAGTGGAGAGCTGAAAGT | RLSTLHITAAV | TCAGACAGACT | AGEPDSNYQLI | | | | | C | |
| | GGAACAAAACCCTCTGTTCC | HDLSATYFCAG | GTATTGGTACA | WGAGTKLIIKP | | | | | (SEQ | |
| | TGAGCATGCAGGAGGGAAA | EPDSNYQLIWG | GGCAGGATCCT | (SEQ ID NO: | | | | | ID NO: | |
| | AAACTATACCATCTACTGCA | AGTKLIIKPDIQ | GGGAAAAGTC | 497) | | | | | 48) | |
| | ATTATTCAACCACTTCAGAC | NPDPAVYQLR | TGGAATCTCTG | | | | | | | |
| | AGACTGTATTGGTACAGGCA | DSKSSDKSVCL | TTTGTGTTGCT | | | | | | | |
| | GGATCCTGGGAAAAGTCTGG | FTDFDSQTNVS | ATCAAATGGA | | | | | | | |
| | AATCTCTGTTTGTGTTGCTAT | QSKDSDVYITD | GCAGTGAAGC | | | | | | | |
| | CAAATGGAGCAGTGAAGCA | KTVLDMRSMD | AGGAGGGACG | | | | | | | |
| | GGAGGGACGATTAATGGCCT | FKSNSAVAWS | ATTAATGGCCT | | | | | | | |
| | CACTTGATACCAAAGCCCGT | NKSDFACANA | CACTTGATACC | | | | | | | |
| | CTCAGCACCCTCCACATCAC | FNNSIIPEDTFF | AAAGCCCGTCT | | | | | | | |
| | AGCTGCCGTGCATGACCTCT | PSPESSCDVKL | CAGCACCCTCC | | | | | | | |
| | CTGCCACCTACTTCTGTGCCG | VEKSFETDTNL | ACATCACAGCT | | | | | | | |
| | GTGAACCTGATAGCAACTAT | NFQNLSVIGFR | GCCGTGCATGA | | | | | | | |
| | CAGTTAATCTGGGGCGCTGG | ILLLKVAGFNL | CCTCTCTGCCA | | | | | | | |
| | GACCAAGCTAATTATAAAGC | LMTLRLWSS | CCTACTTCTGT | | | | | | | |
| | CAGATATCCAGAACCCTGA | (SEQ ID NO: | GCCGGTGAAC | | | | | | | |
| | CCCTGCCGTGTACCAGCTG | 495) | CTGATAGCAAC | | | | | | | |
| | AGAGACTCTAAATCCAGTG | | TATCAGTTAAT | | | | | | | |
| | ACAAGTCTGTCTGCCTATT | | CTGGGGCGCTG | | | | | | | |
| | CACCGATTTTGATTCTCAA | | GGACCAAGCT | | | | | | | |
| | ACAAATGTGTCACAAAGTA | | AATTATAAAGC | | | | | | | |
| | AGGATTCTGATGTGTATAT | | CAG | | | | | | | |
| | CACAGACAAAACTGTGCTA | | (SEQ ID NO: | | | | | | | |
| | GACATGAGGTCTATGGACT | | 496) | | | | | | | |
| | TCAAGAGCAACAGTGCTGT | | | | | | | | | |
| | GGCCTGGAGCAACAAATCT | | | | | | | | | |
| | GACTTTGCATGTGCAAACG | | | | | | | | | |
| | CCTTCAACAACAGCATTAT | | | | | | | | | |
| | TCCAGAAGACACCTTCTTC | | | | | | | | | |
| | CCCAGCCCAGAAAGTTCCT | | | | | | | | | |
| | GTGATGTCAAGCTGGTCGA | | | | | | | | | |
| | GAAAAGCTTTGAAACAGAT | | | | | | | | | |
| | ACGAACCTAAACTTTCAAA | | | | | | | | | |
| | ACCTGTCAGTGATTGGGTT | | | | | | | | | |
| | CCGAATCCTCCTCCTGAAA | | | | | | | | | |
| | GTGGCCGGGTTTAATCTGC | | | | | | | | | |
| | TCATGACGCTGCGGCTGTG | | | | | | | | | |
| | GTCCAGC (SEQ ID NO: 43) | | | | | | | | | |
| 8 | AGAGAGAGAGAGAGAGAGA | MMKSLRVLLVI | CAGAAGGAGG | QKEVEQNSGPL | GACC | DRGS | ATAT | IYSNG | GCCG | CAVG |
| | GGAGGAGGAGGAGGAGGAG | LWLQLSWVWS | TGGAGCAGAA | SVPEGAIASLNC | GAGG | QS | ACTC | D | TGGG | GAGG |
| | GAAGGATGAGGGGAGGGGC | QQKEVEQNSGP | TTCTGGACCCC | TYSDRGSQSFF | TTCCC | (SEQ | CAAT | (SEQ | GGGG | YQKV |
| | TTAGTGGTGAATTAGGGGTG | LSVPEGAIASLN | TCAGTGTTCCA | WYRQYSGKSPE | AGTC | ID NO: | GGTG | ID NO: | GCCG | TF |
| | TTAAAAAGAGCATCATTTTT | CTYSDRGSQSF | GAGGGAGCCA | LIMSIYSNGDKE | C | 52) | AC | 54) | GGGG | (SEQ |
| | TTGAACTGGTAAAGCAGATT | FWYRQYSGKSP | TTGCCTCTCTC | DGRFTAQLNKA | (SEQ | | (SEQ | | TTAC | ID NO: |
| | CTTTTTATGATTTTTAAAGTA | ELIMSIYSNGDK | AACTGCACTTA | SQYVSLLIRDSQ | ID NO: | | ID NO: | | CAGA | 56) |
| | GAAATATCCATTCCAGGTGC | EDGRFTAQLNK | CAGTGACCGA | PSDSATYLCAV | 51) | | 53) | | AAGT | |
| | ATTTTTTAAGGGTTTAAAATT | ASQYVSLLIRDS | GGTTCCCAGTC | GGAGGYQKVT | | | | | TACC | |
| | TGAATCCTCAGTGAACCAGG | QPSDSATYLCA | CTTCTTCTGGT | FGTGTKLQVIP | | | | | (SEQ | |
| | GCAGAGAAGAATGATGAAA | VGGAGGYQKV | ACAGACAATA | (SEQ ID NO: | | | | | ID NO: | |
| | TCCTTGAGAGTTTTACTAGTG | TFGTGTKLQVIP | TTCTGGGAAAA | 500) | | | | | 55) | |
| | ATCCTGTGGCTTCAGTTGAG | NIQNPDPAVYQ | GCCCTGAGTTG | | | | | | | |
| | CTGGGTTTGGAGCCAACAGA | LRDSKSSDKSV | ATAATGTCCAT | | | | | | | |
| | AGGAGGTGGAGCAGAATTCT | CLFTDFDSQTN | ATACTCCAATG | | | | | | | |
| | GGACCCCTCAGTGTTCCAGA | VSQSKDSDVYI | GTGACAAAGA | | | | | | | |
| | GGGAGCCATTGCCTCTCTCA | TDKTVLDMRS | AGATGGAAGG | | | | | | | |
| | ACTGCACTTACAGTGACCGA | MDFKSNSAVA | TTTACAGCACA | | | | | | | |
| | GGTTCCCAGTCCTTCTTCTGG | WSNKSDFACA | GCTCAATAAA | | | | | | | |
| | TACAGACAATATTCTGGGAA | NAFNNSIIPEDT | GCCAGCCAGT | | | | | | | |
| | AAGCCCTGAGTTGATAATGT | FFPSPESSCDV | ATGTTTCTCTG | | | | | | | |
| | CCATATACTCCAATGGTGAC | KLVEKSFETDT | CTCATCAGAGA | | | | | | | |
| | AAAGAAGATGGAAGGTTTAC | NLNFQNLSVIG | CTCCCAGCCCA | | | | | | | |
| | AGCACAGCTCAATAAAGCCA | FRILLLKVAGF | GTGATTCAGCC | | | | | | | |
| | GCCAGTATGTTTCTCTGCTCA | NLLMTLRLWS | ACCTACCTCTG | | | | | | | |
| | TCAGAGACTCCCAGCCCAGT | S | TGCCGTGGGG | | | | | | | |
| | GATTCAGCCACCTACCTCTG | (SEQ ID NO: | GGGGCCGGGG | | | | | | | |
| | TGCCGTGGGGGGGGCCGGGG | 498) | GTTACCAGAA | | | | | | | |
| | GTTACCAGAAAGTTACCTTT | | AGTTACCTTTG | | | | | | | |
| | GGAACTGGAACAAAGCTCCA | | GAACTGGAAC | | | | | | | |
| | AGTCATCCCAAATATCCAGA | | AAAGCTCCAA | | | | | | | |
| | ACCCTGACCCTGCCGTGTA | | GTCATCCCAA | | | | | | | |
| | CCAGCTGAGAGACTCTAAA | | (SEQ ID NO: | | | | | | | |
| | TCCAGTGACAAGTCTGTCT | | 499) | | | | | | | |
| | GCCTATTCACCGATTTTGA | | | | | | | | | |
| | TTCTCAAACAAATGTGTCA | | | | | | | | | |
| | CAAAGTAAGGATTCTGATG | | | | | | | | | |
| | TGTATATCACAGACAAAAC | | | | | | | | | |
| | TGTGCTAGACATGAGGTCT | | | | | | | | | |
| | ATGGACTTCAAGAGCAACA | | | | | | | | | |
| | GTGCTGTGGCCTGGAGCAA | | | | | | | | | |
| | CAAATCTGACTTTGCATGT | | | | | | | | | |
| | GCAAACGCCTTCAACAACA | | | | | | | | | |
| | GCATTATTCCAGAAGACAC | | | | | | | | | |
| | CTTCTTCCCCAGCCCAGAA | | | | | | | | | |
| | AGTTCCTGTGATGTCAAGC | | | | | | | | | |
| | TGGTCGAGAAAAGCTTTGA | | | | | | | | | |
| | AACAGATACGAACCTAAAC | | | | | | | | | |
| | TTTCAAAACCTGTCAGTGA | | | | | | | | | |
| | TTGGGTTCCGAATCCTCCT | | | | | | | | | |
| | CCTGAAAGTGGCCGGGTTT | | | | | | | | | |
| | AATCTGCTCATGACGCTGC | | | | | | | | | |
| | GGCTGTGGTCCAGC (SEQ | | | | | | | | | |
| | ID NO: 50) | | | | | | | | | |
| 9 | GTCCTCCCTTTGTATTTCTAC | MQCQAHGILQQ | GGACAAAGCC | GQSLEQPSEVT | ACAT | TSGFY | AATG | NALD | GCTG | CAVR |
| | TGGGTTTTGCATCCGGACTG | MWGAFLLYVS | TTGAGCAGCCC | AVEGAIVQINC | CTGG | G | CTCT | GL | TGAG | DPL |
| | ATCTTCCTTCCCTCACCCACA | MKMGGTAGQS | TCTGAAGTGAC | TYQTSGFYGLS | GTTTT | (SEQ | GGAT | (SEQ | AGAT | SG |
| | TGAAGTGTCTACCTTCTGCA | LEQPSEVTAVE | AGCTGTGGAA | WYQQHDGGAP | ATGG | ID NO: | GGTT | ID NO: | CCTCT | GYNK |
| | GACTACAGTGGCTCAGGAAC | GAIVQINCTYQ | GGAGCCATTGT | TFLSYNALDGL | G | 59) | TG | 61) | TTCTG | LIF |
| | CGGGGATGCAGTGCCAGGCT | TSGFYGLSWYQ | CCAGATAAACT | EETGRFSSFLSR | (SEQ | | (SEQ | | GTGG | (SEQ |
| | CATGGTATCCTGCAGCAGAT | QHDGGAPTFLS | GCACGTACCA | SDSYGYLLLQE | ID NO: | | ID NO: | | CTAC | ID NO: |
| | GTGGGGAGCTTTCCTTCTCTA | YNALDGLEETG | GACATCTGGGT | LQMKDSASYFC | 58) | | 60) | | AATA | 63) |
| | TGTTTCCATGAAGATGGGAG | RFSSFLSRSDSY | TTTATGGGCTG | AVRDPLSGGYN | | | | | AGCT | |
| | GCACTGCAGGACAAAGCCTT | GYLLLQELQMK | TCCTGGTACCA | KLIFGAGTRLA | | | | | GATT | |
| | GAGCAGCCCTCTGAAGTGAC | DSASYFCAVRD | GCAACATGAT | VHP | | | | | (SEQ | |
| | AGCTGTGGAAGGAGCCATTG | PLSGGYNKLIFG | GGCGGAGCAC | (SEQ ID NO: | | | | | ID NO: | |
| | TCCAGATAAACTGCACGTAC | AGTRLAVHPYI | CCACATTTCTT | 503) | | | | | 62) | |
| | CAGACATCTGGGTTTTATGG | QNPDPAVYQL | TCTTACAATGC | | | | | | | |
| | GCTGTCCTGGTACCAGCAAC | RDSKSSDKSVC | TCTGGATGGTT | | | | | | | |
| | ATGATGGCGGAGCACCCACA | LFTDFDSQTNV | TGGAGGAGAC | | | | | | | |
| | TTTCTTTCTTACAATGCTCTG | SQSKDSDVYIT | AGGTCGTTTTT | | | | | | | |
| | GATGGTTTGGAGGAGACAGG | DKTVLDMRSM | CTTCATTCCTT | | | | | | | |
| | TCGTTTTTCTTCATTCCTTAG | DFKSNSAVAW | AGTCGCTCTGA | | | | | | | |
| | TCGCTCTGATAGTTATGGTTA | SNKSDFACANA | TAGTTATGGTT | | | | | | | |
| | CCTCCTTCTACAGGAGCTCC | FNNSIIPEDTFF | ACCTCCTTCTA | | | | | | | |
| | AGATGAAAGACTCTGCCTCT | PSPESSCDVKL | CAGGAGCTCC | | | | | | | |
| | TACTTCTGCGCTGTGAGAGA | VEKSFETDTNL | AGATGAAAGA | | | | | | | |
| | TCCTCTTTCTGGTGGCTACAA | NFQNLSVIGFR | CTCTGCCTCTT | | | | | | | |
| | TAAGCTGATTTTTGGAGCAG | ILLLKVAGFNL | ACTTCTGCGCT | | | | | | | |
| | GGACCAGGCTGGCTGTACAC | LMTLRLWSS | GTGAGAGATC | | | | | | | |
| | CCATATATCCAGAACCCTG | (SEQ ID NO: | CTCTTTCTGGT | | | | | | | |
| | ACCCTGCCGTGTACCAGCT | 501) | GGCTACAATA | | | | | | | |
| | GAGAGACTCTAAATCCAGT | | AGCTGATTTTT | | | | | | | |
| | GACAAGTCTGTCTGCCTAT | | GGAGCAGGGA | | | | | | | |
| | TCACCGATTTTGATTCTCA | | CCAGGCTGGCT | | | | | | | |
| | AACAAATGTGTCACAAAGT | | GTACACCCAT | | | | | | | |
| | AAGGATTCTGATGTGTATA | | (SEQ ID NO: | | | | | | | |
| | TCACAGACAAAACTGTGCT | | 502) | | | | | | | |
| | AGACATGAGGTCTATGGAC | | | | | | | | | |
| | TTCAAGAGCAACAGTGCTG | | | | | | | | | |
| | TGGCCTGGAGCAACAAATC | | | | | | | | | |
| | TGACTTTGCATGTGCAAAC | | | | | | | | | |
| | GCCTTCAACAACAGCATTA | | | | | | | | | |
| | TTCCAGAAGACACCTTCTT | | | | | | | | | |
| | CCCCAGCCCAGAAAGTTCC | | | | | | | | | |
| | TGTGATGTCAAGCTGGTCG | | | | | | | | | |
| | AGAAAAGCTTTGAAACAGA | | | | | | | | | |
| | TACGAACCTAAACTTTCAA | | | | | | | | | |
| | AACCTGTCAGTGATTGGGT | | | | | | | | | |
| | TCCGAATCCTCCTCCTGAA | | | | | | | | | |
| | AGTGGCCGGGTTTAATCTG | | | | | | | | | |
| | CTCATGACGCTGCGGCTGT | | | | | | | | | |
| | GGTCCAGC (SEQ ID NO: 57) | | | | | | | | | |
| 10 | GAGCACTCAAATTGAAACCT | MKLVTSITVLLS | GATGCTAAGA | DAKTTQPNSME | ACAA | TISGT | GGTC | GLTSN | ATCC | CILYS |
| | GCCTGATGTGGGATGTGCTG | LGIMGDAKTTQ | CCACACAGCC | SNEEEPVHLPC | TCAG | DY | TTAC | (SEQ | TGTA | GGGA |
| | TGGCTGCTGCTTTGTTGCTTG | PNSMESNEEEP | AAATTCAATGG | NHSTISGTDYIH | TGGA | (SEQ | AAGC | ID NO: | TTCA | DGLTF |
| | GGACCTCCTCTGACCTAGGA | VHLPCNHSTISG | AGAGTAACGA | WYRQLPSQGPE | ACTG | ID NO: | AAT | 68) | GGAG | (SEQ |
| | TCAGACACAGAGTCTGAGTT | TDYIHWYRQLP | AGAAGAGCCT | YVIHGLTSNVN | ATTA | 66) | (SEQ | | GAGG | ID NO: |
| | CTGGGGCCTGGAACCTCAAT | SQGPEYVIHGL | GTTCACTTGCC | NRMASLAIAED | C | | ID NO: | | TGCT | 70) |
| | GTGCACTTGAACAATGAAGT | TSNVNNRMASL | TTGTAACCACT | RKSSTLILHRAT | (SEQ | | 67) | | GACG | |
| | TGGTGACAAGCATTACTGTA | AIAEDRKSSTLI | CCACAATCAGT | LRDAAVYYCIL | ID NO: | | | | GACT | |
| | CTCCTATCTTTGGGTATTATG | LHRATLRDAAV | GGAACTGATTA | YSGGGADGLTF | 65) | | | | CACC | |
| | GGTGATGCTAAGACCACACA | YYCILYSGGGA | CATACATTGGT | GKGTHLIIQP | | | | | (SEQ | |
| | GCCAAATTCAATGGAGAGTA | DGLTFGKGTHL | ATCGACAGCTT | (SEQ ID NO: | | | | | ID NO: | |
| | ACGAAGAAGAGCCTGTTCAC | IIQPYIQNPDPA | CCCTCCCAGGG | 506) | | | | | 69) | |
| | TTGCCTTGTAACCACTCCAC | VYQLRDSKSSD | TCCAGAGTACG | | | | | | | |
| | AATCAGTGGAACTGATTACA | KSVCLFTDFDS | TGATTCATGGT | | | | | | | |
| | TACATTGGTATCGACAGCTT | QTNVSQSKDSD | CTTACAAGCAA | | | | | | | |
| | CCCTCCCAGGGTCCAGAGTA | VYITDKTVLD | TGTGAACAAC | | | | | | | |
| | CGTGATTCATGGTCTTACAA | MRSMDFKSNS | AGAATGGCCTC | | | | | | | |
| | GCAATGTGAACAACAGAATG | AVAWSNKSDF | TCTGGCAATCG | | | | | | | |
| | GCCTCTCTGGCAATCGCTGA | ACANAFNNSII | CTGAAGACAG | | | | | | | |
| | AGACAGAAAGTCCAGTACCT | PEDTFFPSPESS | AAAGTCCAGT | | | | | | | |
| | TGATCCTGCACCGTGCTACC | CDVKLVEKSF | ACCTTGATCCT | | | | | | | |
| | TTGAGAGATGCTGCTGTGTA | ETDTNLNFQN | GCACCGTGCTA | | | | | | | |
| | CTACTGCATCCTGTATTCAG | LSVIGFRILLL | CCTTGAGAGAT | | | | | | | |
| | GAGGAGGTGCTGACGGACTC | KVAGFNLLMT | GCTGCTGTGTA | | | | | | | |
| | ACCTTTGGCAAAGGGACTCA | LRLWSS | CTACTGCATCC | | | | | | | |
| | TCTAATCATCCAGCCCTATA | (SEQ ID NO: | TGTATTCAGGA | | | | | | | |
| | TCCAGAACCCTGACCCTGC | 504) | GGAGGTGCTG | | | | | | | |
| | CGTGTACCAGCTGAGAGAC | | ACGGACTCACC | | | | | | | |
| | TCTAAATCCAGTGACAAGT | | TTTGGCAAAGG | | | | | | | |
| | CTGTCTGCCTATTCACCGA | | GACTCATCTAA | | | | | | | |
| | TTTTGATTCTCAAACAAAT | | TCATCCAGCCC | | | | | | | |
| | GTGTCACAAAGTAAGGATT | | T | | | | | | | |
| | CTGATGTGTATATCACAGA | | (SEQ ID NO: | | | | | | | |
| | CAAAACTGTGCTAGACATG | | 505) | | | | | | | |
| | AGGTCTATGGACTTCAAGA | | | | | | | | | |
| | GCAACAGTGCTGTGGCCTG | | | | | | | | | |
| | GAGCAACAAATCTGACTTT | | | | | | | | | |
| | GCATGTGCAAACGCCTTCA | | | | | | | | | |
| | ACAACAGCATTATTCCAGA | | | | | | | | | |
| | AGACACCTTCTTCCCCAGC | | | | | | | | | |
| | CCAGAAAGTTCCTGTGATG | | | | | | | | | |
| | TCAAGCTGGTCGAGAAAAG | | | | | | | | | |
| | CTTTGAAACAGATACGAAC | | | | | | | | | |
| | CTAAACTTTCAAAACCTGT | | | | | | | | | |
| | CAGTGATTGGGTTCCGAAT | | | | | | | | | |
| | CCTCCTCCTGAAAGTGGCC | | | | | | | | | |
| | GGGTTTAATCTGCTCATGA | | | | | | | | | |
| | CGCTGCGGCTGTGGTCCAG | | | | | | | | | |
| | C (SEQ ID NO: 64) | | | | | | | | | |
| 11 | TTCCTGTCTTGAGAAGAGAA | MAQELGMQCQ | GGACAAAACA | GQNIDQPTEMT | ACAT | TSGFN | AATG | NVLD | GCTG | CAGL |
| | ACACTTATGGGGAAATTGAA | ARGILQQMWG | TTGACCAGCCC | ATEGAIVQINCT | CTGG | G | TTCTG | GL | GCCT | FGNTP |
| | TACTTTGTCTTTTTTTTTTTTT | VFLLYVSMKM | ACTGAGATGA | YQTSGFNGLFW | GTTC | (SEQ | GATG | (SEQ | CTTC | LVF |
| | TTTTTGAGACGGAGTCTCAC | GGTTGQNIDQP | CAGCTACGGA | YQQHAGEAPTF | AACG | ID NO: | GTTT | ID NO: | GGAA | (SEQ |
| | TTTGTCGCCCAGGCTGGAGT | TEMTATEGAIV | AGGTGCCATTG | LSYNVLDGLEE | GG | 73) | G | 75) | ACAC | ID NO: |
| | GCAGTGGCGCGATCTCTGCT | QINCTYQTSGF | TCCAGATCAAC | KGRFSSFLSRSK | (SEQ | | (SEQ | | ACCT | 77) |
| | CACTGCAAACTCCGCCTCCC | NGLFWYQQHA | TGCACGTACCA | GYSYLLLKELQ | ID NO: | | ID NO: | | CTTGT | |
| | GGGTTCCCGCCATTCTCCTGC | GEAPTFLSYNV | GACATCTGGGT | MKDSASYLCA | 72) | | 74) | | C | |
| | CTCAGCCTCTCGAGTAGCTG | LDGLEEKGRFS | TCAACGGGCTG | GLFGNTPLVFG | | | | | (SEQ | |
| | GGACTACAGGCGCCCGCCAC | SFLSRSKGYSYL | TTCTGGTACCA | KGTRLSVIA | | | | | ID NO: | |
| | AGCGCCCAGCTAATTTTTTTG | LLKELQMKDSA | GCAACATGCTG | (SEQ ID NO: | | | | | 76) | |
| | TATTTTTTGGTAGAGACGGG | SYLCAGLFGNT | GCGAAGCACC | 509) | | | | | | |
| | GTTTCACCGTGTTAGCCAGG | PLVFGKGTRLS | CACATTTCTGT | | | | | | | |
| | ATGGTCTCGATCTCCTGACCT | VIANIQNPDPA | CTTACAATGTT | | | | | | | |
| | CGTGATCCGCCCACCTCGGC | VYQLRDSKSSD | CTGGATGGTTT | | | | | | | |
| | CTCCCAAAGCGCTGGGATTA | KSVCLFTDFDS | GGAGGAGAAA | | | | | | | |
| | AAGGCGTGAGCCACCGCGCC | QTNVSQSKDSD | GGTCGTTTTTC | | | | | | | |
| | CGGCCTACTTTGTCTTATGTT | VYITDKTVLD | TTCATTCCTTA | | | | | | | |
| | ATTCCCATTTGCCGTCTCTGT | MRSMDFKSNS | GTCGGTCTAAA | | | | | | | |
| | TCCTTATACATTATGCTTTTT | AVAWSNKSDF | GGGTACAGTTA | | | | | | | |
| | CAACTTTACCAGAATCACTT | ACANAFNNSII | CCTCCTTTTGA | | | | | | | |
| | GGATTAAAACCCGTGGATTT | PEDTFFPSPESS | AGGAGCTCCA | | | | | | | |
| | CTCAGTAGGAAATGTTCATG | CDVKLVEKSF | GATGAAAGAC | | | | | | | |
| | TGAAGACACTTCTGTAGTAA | ETDTNLNFQN | TCTGCCTCTTA | | | | | | | |
| | CAGAACCTACAGCTGCTCCT | LSVIGFRILLL | CCTCTGTGCTG | | | | | | | |
| | GTAGAAGGAAGTTGAAAGTC | KVAGFNLLMT | GCCTCTTCGGA | | | | | | | |
| | ATCCCTTCAAGAAAGGGGCT | LRLWSS | AACACACCTCT | | | | | | | |
| | CCTCCCCTTGTAATTCTACTG | (SEQ ID NO: | TGTCTTTGGAA | | | | | | | |
| | GGTTTTGCATCCAGACTGAG | 507) | AGGGCACAAG | | | | | | | |
| | TTTCCTTCCCTCACCCACATG | | ACTTTCTGTGA | | | | | | | |
| | AAGTGTCTACCTTCTGCAGA | | TTGCAA | | | | | | | |
| | CTCCAATGGCTCAGGAACTG | | (SEQ ID NO: | | | | | | | |
| | GGAATGCAGTGCCAGGCTCG | | 508) | | | | | | | |
| | TGGTATCCTGCAGCAGATGT | | | | | | | | | |
| | GGGGAGTTTTCCTTCTTTATG | | | | | | | | | |
| | TTTCCATGAAGATGGGAGGC | | | | | | | | | |
| | ACTACAGGACAAAACATTGA | | | | | | | | | |
| | CCAGCCCACTGAGATGACAG | | | | | | | | | |
| | CTACGGAAGGTGCCATTGTC | | | | | | | | | |
| | CAGATCAACTGCACGTACCA | | | | | | | | | |
| | GACATCTGGGTTCAACGGGC | | | | | | | | | |
| | TGTTCTGGTACCAGCAACAT | | | | | | | | | |
| | GCTGGCGAAGCACCCACATT | | | | | | | | | |
| | TCTGTCTTACAATGTTCTGGA | | | | | | | | | |
| | TGGTTTGGAGGAGAAAGGTC | | | | | | | | | |
| | GTTTTTCTTCATTCCTTAGTC | | | | | | | | | |
| | GGTCTAAAGGGTACAGTTAC | | | | | | | | | |
| | CTCCTTTTGAAGGAGCTCCA | | | | | | | | | |
| | GATGAAAGACTCTGCCTCTT | | | | | | | | | |
| | ACCTCTGTGCTGGCCTCTTCG | | | | | | | | | |
| | GAAACACACCTCTTGTCTTT | | | | | | | | | |
| | GGAAAGGGCACAAGACTTTC | | | | | | | | | |
| | TGTGATTGCAAATATCCAGA | | | | | | | | | |
| | ACCCTGACCCTGCCGTGTA | | | | | | | | | |
| | CCAGCTGAGAGACTCTAAA | | | | | | | | | |
| | TCCAGTGACAAGTCTGTCT | | | | | | | | | |
| | GCCTATTCACCGATTTTGA | | | | | | | | | |
| | TTCTCAAACAAATGTGTCA | | | | | | | | | |
| | CAAAGTAAGGATTCTGATG | | | | | | | | | |
| | TGTATATCACAGACAAAAC | | | | | | | | | |
| | TGTGCTAGACATGAGGTCT | | | | | | | | | |
| | ATGGACTTCAAGAGCAACA | | | | | | | | | |
| | GTGCTGTGGCCTGGAGCAA | | | | | | | | | |
| | CAAATCTGACTTTGCATGT | | | | | | | | | |
| | GCAAACGCCTTCAACAACA | | | | | | | | | |
| | GCATTATTCCAGAAGACAC | | | | | | | | | |
| | CTTCTTCCCCAGCCCAGAA | | | | | | | | | |
| | AGTTCCTGTGATGTCAAGC | | | | | | | | | |
| | TGGTCGAGAAAAGCTTTGA | | | | | | | | | |
| | AACAGATACGAACCTAAAC | | | | | | | | | |
| | TTTCAAAACCTGTCAGTGA | | | | | | | | | |
| | TTGGGTTCCGAATCCTCCT | | | | | | | | | |
| | CCTGAAAGTGGCCGGGTTT | | | | | | | | | |
| | AATCTGCTCATGACGCTGC | | | | | | | | | |
| | GGCTGTGGTCCAGC (SEQ | | | | | | | | | |
| | ID NO: 71) | | | | | | | | | |
| 12 | TGGGGCTGTTCTGAAGCAGC | MACPGFLWAL | GCTCAGACAGT | AQTVTQSQPEM | ACCA | TSESD | CAAG | QEAY | GCTT | CAYF |
| | TACGGCACCAGTGCAGCTGA | VISTCLEFSMAQ | CACTCAGTCTC | SVQEAETVTLS | GTGA | YY | AAGC | KQQN | ATTTT | NTDK |
| | TACTCAAGGTTCAGATCAGA | TVTQSQPEMSV | AACCAGAGAT | CTYDTSESDYY | GAGT | (SEQ | TTAT | (SEQ | AACA | LIF |
| | AGAGGAGGCTTCTCACCCTG | QEAETVTLSCT | GTCTGTGCAGG | LFWYKQPPSRQ | GATT | ID NO: | AAGC | ID NO: | CCGA | (SEQ |
| | CAGCAGGGACCTGTGAGCAT | YDTSESDYYLF | AGGCAGAGAC | MILVIRQEAYK | ATTA | 80) | AACA | 82) | CAAG | ID NO: |
| | GGCATGCCCTGGCTTCCTGT | WYKQPPSRQMI | CGTGACCCTGA | QQNATENRFSV | T | | GAAT | | CTCA | 84) |
| | GGGCACTTGTGATCTCCACC | LVIRQEAYKQQ | GCTGCACATAT | NFQKAAKSFSL | (SEQ | | (SEQ | | TC | |
| | TGTCTTGAATTTAGCATGGCT | NATENRFSVNF | GACACCAGTG | KISDSQLGDAA | ID NO: | | ID NO: | | (SEQ | |
| | CAGACAGTCACTCAGTCTCA | QKAAKSFSLKIS | AGAGTGATTAT | MYFCAYFNTD | 79) | | 81) | | ID NO: | |
| | ACCAGAGATGTCTGTGCAGG | DSQLGDAAMY | TATTTATTCTG | KLIFGTGTRLQ | | | | | 83) | |
| | AGGCAGAGACCGTGACCCTG | FCAYFNTDKLIF | GTACAAGCAG | VFP | | | | | | |
| | AGCTGCACATATGACACCAG | GTGTRLQVFPN | CCTCCCAGCAG | (SEQ ID NO: | | | | | | |
| | TGAGAGTGATTATTATTTATT | IQNPDPAVYQL | GCAGATGATTC | 512) | | | | | | |
| | CTGGTACAAGCAGCCTCCCA | RDSKSSDKSVC | TCGTTATTCGC | | | | | | | |
| | GCAGGCAGATGATTCTCGTT | LFTDFDSQTNV | CAAGAAGCTT | | | | | | | |
| | ATTCGCCAAGAAGCTTATAA | SQSKDSDVYIT | ATAAGCAACA | | | | | | | |
| | GCAACAGAATGCAACAGAG | DKTVLDMRSM | GAATGCAACA | | | | | | | |
| | AATCGTTTCTCTGTGAACTTC | DFKSNSAVAW | GAGAATCGTTT | | | | | | | |
| | CAGAAAGCAGCCAAATCCTT | SNKSDFACANA | CTCTGTGAACT | | | | | | | |
| | CAGTCTCAAGATCTCAGACT | FNNSIIPEDTFF | TCCAGAAAGC | | | | | | | |
| | CACAGCTGGGGGATGCCGCG | PSPESSCDVKL | AGCCAAATCCT | | | | | | | |
| | ATGTATTTCTGTGCTTATTTT | VEKSFETDTNL | TCAGTCTCAAG | | | | | | | |
| | AACACCGACAAGCTCATCTT | NFQNLSVIGFR | ATCTCAGACTC | | | | | | | |
| | TGGGACTGGGACCAGATTAC | ILLLKVAGFNL | ACAGCTGGGG | | | | | | | |
| | AAGTCTTTCCAAATATCCAG | LMTLRLWSS | GATGCCGCGAT | | | | | | | |
| | AACCCTGACCCTGCCGTGT | (SEQ ID NO: | GTATTTCTGTG | | | | | | | |
| | ACCAGCTGAGAGACTCTAA | 510) | CTTATTTTAAC | | | | | | | |
| | ATCCAGTGACAAGTCTGTC | | ACCGACAAGC | | | | | | | |
| | TGCCTATTCACCGATTTTG | | TCATCTTTGGG | | | | | | | |
| | ATTCTCAAACAAATGTGTC | | ACTGGGACCA | | | | | | | |
| | ACAAAGTAAGGATTCTGAT | | GATTACAAGTC | | | | | | | |
| | GTGTATATCACAGACAAAA | | TTTCCAA | | | | | | | |
| | CTGTGCTAGACATGAGGTC | | (SEQ ID NO: | | | | | | | |
| | TATGGACTTCAAGAGCAAC | | 511) | | | | | | | |
| | AGTGCTGTGGCCTGGAGCA | | | | | | | | | |
| | ACAAATCTGACTTTGCATG | | | | | | | | | |
| | TGCAAACGCCTTCAACAAC | | | | | | | | | |
| | AGCATTATTCCAGAAGACA | | | | | | | | | |
| | CCTTCTTCCCCAGCCCAGA | | | | | | | | | |
| | AAGTTCCTGTGATGTCAAG | | | | | | | | | |
| | CTGGTCGAGAAAAGCTTTG | | | | | | | | | |
| | AAACAGATACGAACCTAAA | | | | | | | | | |
| | CTTTCAAAACCTGTCAGTG | | | | | | | | | |
| | ATTGGGTTCCGAATCCTCC | | | | | | | | | |
| | TCCTGAAAGTGGCCGGGTT | | | | | | | | | |
| | TAATCTGCTCATGACGCTG | | | | | | | | | |
| | CGGCTGTGGTCCAGC (SEQ | | | | | | | | | |
| | ID NO: 78) | | | | | | | | | |
| 13 | GGCACACACACACATGAATA | MMKSLRVLLVI | CAGAAGGAGG | QKEVEQDPGPL | AACA | NSAF | ACAT | TYSSG | GCAA | CAMG |
| | AGAGCAAGACAGAGGGAGA | LWLQLSWVWS | TGGAGCAGGA | SVPEGAIVSLNC | GTGC | QY | ACTC | N | TGGG | GQGG |
| | GATGAGGGAGGAGCTTAATG | QQKEVEQDPGP | TCCTGGACCAC | TYSNSAFQYFM | TTTTC | (SEQ | CAGT | (SEQ | GGGC | ADGL |
| | ATGGAGCAGAGGTGTTAAAA | LSVPEGAIVSLN | TCAGTGTTCCA | WYRQYSRKGP | AATA | ID NO: | GGTA | ID NO: | CAAG | TF |
| | AGAACATCCTTTTTCTAATTG | CTYSNSAFQYF | GAGGGAGCCA | ELLMYTYSSGN | C | 87) | AC | 89) | GAGG | (SEQ |
| | GTAGGACAGATTTCTTTTAT | MWYRQYSRKG | TTGTTTCTCTC | KEDGRFTAQVD | (SEQ | | (SEQ | | TGCT | ID NO: |
| | GATTCCTACAGCAGAAAAAT | PELLMYTYSSG | AACTGCACTTA | KSSKYISLFIRD | ID NO: | | ID NO: | | GACG | 91) |
| | GAGAAACGTTTGTTATTATTT | NKEDGRFTAQV | CAGCAACAGT | SQPSDSATYLC | 86) | | 88) | | GACT | |
| | TTTTTTCGTGTTTAAGGTTTG | DKSSKYISLFIR | GCTTTTCAATA | AMGGQGGADG | | | | | CACC | |
| | AATCCTCAGTGAACCAGGGC | DSQPSDSATYL | CTTCATGTGGT | LTFGKGTHLIIQ | | | | | (SEQ | |
| | AGAAAAGAATGATGAAATCC | CAMGGQGGAD | ACAGACAGTA | P | | | | | ID NO: | |
| | TTGAGAGTTTTACTGGTGAT | GLTFGKGTHLII | TTCCAGAAAA | (SEQ ID NO: | | | | | 90) | |
| | CCTGTGGCTTCAGTTAAGCT | QPYIQNPDPAV | GGCCCTGAGTT | 515) | | | | | | |
| | GGGTTTGGAGCCAACAGAAG | YQLRDSKSSDK | GCTGATGTACA | | | | | | | |
| | GAGGTGGAGCAGGATCCTGG | SVCLFTDFDSQ | CATACTCCAGT | | | | | | | |
| | ACCACTCAGTGTTCCAGAGG | TNVSQSKDSDV | GGTAACAAAG | | | | | | | |
| | GAGCCATTGTTTCTCTCAACT | YITDKTVLDM | AAGATGGAAG | | | | | | | |
| | GCACTTACAGCAACAGTGCT | RSMDFKSNSA | GTTTACAGCAC | | | | | | | |
| | TTTCAATACTTCATGTGGTAC | VAWSNKSDFA | AGGTCGATAA | | | | | | | |
| | AGACAGTATTCCAGAAAAGG | CANAFNNSIIPE | ATCCAGCAAGT | | | | | | | |
| | CCCTGAGTTGCTGATGTACA | DTFFPSPESSC | ATATCTCCTTG | | | | | | | |
| | CATACTCCAGTGGTAACAAA | DVKLVEKSFET | TTCATCAGAGA | | | | | | | |
| | GAAGATGGAAGGTTTACAGC | DTNLNFQNLSV | CTCACAGCCCA | | | | | | | |
| | ACAGGTCGATAAATCCAGCA | IGFRILLLKVA | GTGATTCAGCC | | | | | | | |
| | AGTATATCTCCTTGTTCATCA | GFNLLMTLRL | ACCTACCTCTG | | | | | | | |
| | GAGACTCACAGCCCAGTGAT | WSS | TGCAATGGGG | | | | | | | |
| | TCAGCCACCTACCTCTGTGC | (SEQ ID NO: | GGCCAAGGAG | | | | | | | |
| | AATGGGGGGCCAAGGAGGT | 513) | GTGCTGACGG | | | | | | | |
| | GCTGACGGACTCACCTTTGG | | ACTCACCTTTG | | | | | | | |
| | CAAAGGGACTCATCTAATCA | | GCAAAGGGAC | | | | | | | |
| | TCCAGCCCTATATCCAGAAC | | TCATCTAATCA | | | | | | | |
| | CCTGACCCTGCCGTGTACC | | TCCAGCCCT | | | | | | | |
| | AGCTGAGAGACTCTAAATC | | (SEQ ID NO: | | | | | | | |
| | CAGTGACAAGTCTGTCTGC | | 514) | | | | | | | |
| | CTATTCACCGATTTTGATT | | | | | | | | | |
| | CTCAAACAAATGTGTCACA | | | | | | | | | |
| | AAGTAAGGATTCTGATGTG | | | | | | | | | |
| | TATATCACAGACAAAACTG | | | | | | | | | |
| | TGCTAGACATGAGGTCTAT | | | | | | | | | |
| | GGACTTCAAGAGCAACAGT | | | | | | | | | |
| | GCTGTGGCCTGGAGCAACA | | | | | | | | | |
| | AATCTGACTTTGCATGTGC | | | | | | | | | |
| | AAACGCCTTCAACAACAGC | | | | | | | | | |
| | ATTATTCCAGAAGACACCT | | | | | | | | | |
| | TCTTCCCCAGCCCAGAAAG | | | | | | | | | |
| | TTCCTGTGATGTCAAGCTG | | | | | | | | | |
| | GTCGAGAAAAGCTTTGAAA | | | | | | | | | |
| | CAGATACGAACCTAAACTT | | | | | | | | | |
| | TCAAAACCTGTCAGTGATT | | | | | | | | | |
| | GGGTTCCGAATCCTCCTCC | | | | | | | | | |
| | TGAAAGTGGCCGGGTTTAA | | | | | | | | | |
| | TCTGCTCATGACGCTGCGG | | | | | | | | | |
| | CTGTGGTCCAGC (SEQ ID | | | | | | | | | |
| | NO: 85) | | | | | | | | | |
| 14 | GGGAGTGTCACTCTAAGCCC | MLLEHLLIILW | GGTCAACAGCT | GQQLNQSPQSM | AGCA | SIFNT | TTAT | LYKA | GCTG | CAGP |
| | AAGAGAGTTTCTTGAAGCAA | MQLTWVSGQQ | GAATCAGAGT | FIQEGEDVSMN | TATTT | (SEQ | ATAA | GEL | GCCC | KREY |
| | AAAAAAAAAAAAACCCATTC | LNQSPQSMFIQE | CCTCAATCTAT | CTSSSIFNTWL | AACA | ID NO: | GGCT | (SEQ | CAAG | GNKL |
| | AGGAAATAATTCTTTGCTGA | GEDVSMNCTSS | GTTTATCCAGG | WYKQDPGEGP | CC | 94) | GGTG | ID NO: | AGGG | VF |
| | TAAGGATGCTCCTTGAACAT | SIFNTWLWYKQ | AAGGAGAAGA | VLLIALYKAGE | (SEQ | | AATT | 96) | AATA | (SEQ |
| | TTATTAATAATCTTGTGGATG | DPGEGPVLLIAL | TGTCTCCATGA | LTSNGRLTAQF | ID NO: | | G | | TGGA | ID NO: |
| | CAGCTGACATGGGTCAGTGG | YKAGELTSNGR | ACTGCACTTCT | GITRKDSFLNIS | 93) | | (SEQ | | AACA | 98) |
| | TCAACAGCTGAATCAGAGTC | LTAQFGITRKDS | TCAAGCATATT | ASIPSDVGIYFC | | | ID NO: | | AGCT | |
| | CTCAATCTATGTTTATCCAGG | FLNISASIPSDV | TAACACCTGGC | AGPKREYGNKL | | | 95) | | GGTC | |
| | AAGGAGAAGATGTCTCCATG | GIYFCAGPKRE | TATGGTACAAG | VFGAGTILRVK | | | | | (SEQ | |
| | AACTGCACTTCTTCAAGCAT | YGNKLVFGAGT | CAGGACCCTG | S | | | | | ID NO: | |
| | ATTTAACACCTGGCTATGGT | ILRVKSYIQNPD | GGGAAGGTCC | (SEQ ID NO: | | | | | 97) | |
| | ACAAGCAGGACCCTGGGGA | PAVYQLRDSKS | TGTCCTCTTGA | 518) | | | | | | |
| | AGGTCCTGTCCTCTTGATAG | SDKSVCLFTDF | TAGCCTTATAT | | | | | | | |
| | CCTTATATAAGGCTGGTGAA | DSQTNVSQSKD | AAGGCTGGTG | | | | | | | |
| | TTGACCTCAAATGGAAGACT | SDVYITDKTVL | AATTGACCTCA | | | | | | | |
| | GACTGCTCAGTTTGGTATAA | DMRSMDFKSN | AATGGAAGAC | | | | | | | |
| | CCAGAAAGGACAGCTTCCTG | SAVAWSNKSD | TGACTGCTCAG | | | | | | | |
| | AATATCTCAGCATCCATACC | FACANAFNNSI | TTTGGTATAAC | | | | | | | |
| | TAGTGATGTAGGCATCTACT | IPEDTFFPSPES | CAGAAAGGAC | | | | | | | |
| | TCTGTGCTGGCCCCAAGAGG | SCDVKLVEKSF | AGCTTCCTGAA | | | | | | | |
| | GAATATGGAAACAAGCTGGT | ETDTNLNFQN | TATCTCAGCAT | | | | | | | |
| | CTTTGGCGCAGGAACCATTC | LSVIGFRILLL | CCATACCTAGT | | | | | | | |
| | TGAGAGTCAAGTCCTATATC | KVAGFNLLMT | GATGTAGGCAT | | | | | | | |
| | CAGAACCCTGACCCTGCCG | LRLWSS | CTACTTCTGTG | | | | | | | |
| | TGTACCAGCTGAGAGACTC | (SEQ ID NO: | CTGGCCCCAAG | | | | | | | |
| | TAAATCCAGTGACAAGTCT | 516) | AGGGAATATG | | | | | | | |
| | GTCTGCCTATTCACCGATT | | GAAACAAGCT | | | | | | | |
| | TTGATTCTCAAACAAATGT | | GGTCTTTGGCG | | | | | | | |
| | GTCACAAAGTAAGGATTCT | | CAGGAACCATT | | | | | | | |
| | GATGTGTATATCACAGACA | | CTGAGAGTCA | | | | | | | |
| | AAACTGTGCTAGACATGAG | | AGTCCT | | | | | | | |
| | GTCTATGGACTTCAAGAGC | | (SEQ ID NO: | | | | | | | |
| | AACAGTGCTGTGGCCTGGA | | 517) | | | | | | | |
| | GCAACAAATCTGACTTTGC | | | | | | | | | |
| | ATGTGCAAACGCCTTCAAC | | | | | | | | | |
| | AACAGCATTATTCCAGAAG | | | | | | | | | |
| | ACACCTTCTTCCCCAGCCC | | | | | | | | | |
| | AGAAAGTTCCTGTGATGTC | | | | | | | | | |
| | AAGCTGGTCGAGAAAAGCT | | | | | | | | | |
| | TTGAAACAGATACGAACCT | | | | | | | | | |
| | AAACTTTCAAAACCTGTCA | | | | | | | | | |
| | GTGATTGGGTTCCGAATCC | | | | | | | | | |
| | TCCTCCTGAAAGTGGCCGG | | | | | | | | | |
| | GTTTAATCTGCTCATGACG | | | | | | | | | |
| | CTGCGGCTGTGGTCCAGC | | | | | | | | | |
| | (SEQ ID NO: 92) | | | | | | | | | |
| 15 | GGGGACAACTGTAGCAACCC | MALQSTLGAV | AAGGACCAAG | KDQVFQPSTVA | GTGT | VSNA | GGCT | GSKP | GCTG | CAVE |
| | TTCTAAAGGTTGTAGATTCT | WLGLLLNSLW | TGTTTCAGCCT | SSEGAVVEIFCN | CCAA | YN | CAAA | (SEQ | TGGA | DNNN |
| | GGCTGATGATGTCACTGACA | KVAESKDQVFQ | TCCACAGTGGC | HSVSNAYNFFW | TGCTT | (SEQ | GCCT | ID NO: | GGAT | NDMR |
| | CAAAGGAAAAAATGCAAAA | PSTVASSEGAV | ATCTTCAGAGG | YLHFPGCAPRL | ACAA | ID NO: | (SEQ | 103) | AACA | F |
| | CAGGTAGTCTTAAATAAGCA | VEIFCNHSVSN | GAGCTGTGGTG | LVKGSKPSQQG | C | 101) | ID NQ | | ATAA | (SEQ |
| | TTCTGGTGAGACCAACTGCA | AYNFFWYLHFP | GAAATCTTCTG | RYNMTYERFSS | (SEQ | | 102) | | CAAT | ID NO: |
| | TTTTGGCCATGGCTTTGCAG | GCAPRLLVKGS | TAATCACTCTG | SLLILQVREAD | ID NO: | | | | GACA | 105) |
| | AGCACTCTGGGGGCGGTGTG | KPSQQGRYNM | TGTCCAATGCT | AAVYYCAVED | 100) | | | | TGCG | |
| | GCTAGGGCTTCTCCTCAACT | TYERFSSSLLIL | TACAACTTCTT | NNNNDMRFGA | | | | | C | |
| | CTCTCTGGAAGGTTGCAGAA | QVREADAAVY | CTGGTACCTTC | GTRLTVKP | | | | | (SEQ | |
| | AGCAAGGACCAAGTGTTTCA | YCAVEDNNNN | ACTTCCCGGGA | (SEQ ID NO: | | | | | ID NO: | |
| | GCCTTCCACAGTGGCATCTT | DMRFGAGTRLT | TGTGCACCAAG | 521) | | | | | 104) | |
| | CAGAGGGAGCTGTGGTGGAA | VKPNIQNPDPA | ACTCCTTGTTA | | | | | | | |
| | ATCTTCTGTAATCACTCTGTG | VYQLRDSKSSD | AAGGCTCAAA | | | | | | | |
| | TCCAATGCTTACAACTTCTTC | KSVCLFTDFDS | GCCTTCTCAGC | | | | | | | |
| | TGGTACCTTCACTTCCCGGG | QTNVSQSKDSD | AGGGACGATA | | | | | | | |
| | ATGTGCACCAAGACTCCTTG | VYITDKTVLD | CAACATGACCT | | | | | | | |
| | TTAAAGGCTCAAAGCCTTCT | MRSMDFKSNS | ATGAACGGTTC | | | | | | | |
| | CAGCAGGGACGATACAACAT | AVAWSNKSDF | TCTTCATCGCT | | | | | | | |
| | GACCTATGAACGGTTCTCTT | ACANAFNNSII | GCTCATCCTCC | | | | | | | |
| | CATCGCTGCTCATCCTCCAG | PEDTFFPSPESS | AGGTGCGGGA | | | | | | | |
| | GTGCGGGAGGCAGATGCTGC | CDVKLVEKSF | GGCAGATGCT | | | | | | | |
| | TGTTTACTACTGTGCTGTGGA | ETDTNLNFQN | GCTGTTTACTA | | | | | | | |
| | GGATAACAATAACAATGACA | LSVIGFRILLL | CTGTGCTGTGG | | | | | | | |
| | TGCGCTTTGGAGCAGGGACC | KVAGFNLLMT | AGGATAACAA | | | | | | | |
| | AGACTGACAGTAAAACCAAA | LRLWSS | TAACAATGAC | | | | | | | |
| | TATCCAGAACCCTGACCCT | (SEQ ID NO: | ATGCGCTTTGG | | | | | | | |
| | GCCGTGTACCAGCTGAGAG | 519) | AGCAGGGACC | | | | | | | |
| | ACTCTAAATCCAGTGACAA | | AGACTGACAG | | | | | | | |
| | GTCTGTCTGCCTATTCACC | | TAAAACCAA | | | | | | | |
| | GATTTTGATTCTCAAACAA | | (SEQ ID NO: | | | | | | | |
| | ATGTGTCACAAAGTAAGGA | | 520) | | | | | | | |
| | TTCTGATGTGTATATCACA | | | | | | | | | |
| | GACAAAACTGTGCTAGACA | | | | | | | | | |
| | TGAGGTCTATGGACTTCAA | | | | | | | | | |
| | GAGCAACAGTGCTGTGGCC | | | | | | | | | |
| | TGGAGCAACAAATCTGACT | | | | | | | | | |
| | TTGCATGTGCAAACGCCTT | | | | | | | | | |
| | CAACAACAGCATTATTCCA | | | | | | | | | |
| | GAAGACACCTTCTTCCCCA | | | | | | | | | |
| | GCCCAGAAAGTTCCTGTGA | | | | | | | | | |
| | TGTCAAGCTGGTCGAGAAA | | | | | | | | | |
| | AGCTTTGAAACAGATACGA | | | | | | | | | |
| | ACCTAAACTTTCAAAACCT | | | | | | | | | |
| | GTCAGTGATTGGGTTCCGA | | | | | | | | | |
| | ATCCTCCTCCTGAAAGTGG | | | | | | | | | |
| | CCGGGTTTAATCTGCTCAT | | | | | | | | | |
| | GACGCTGCGGCTGTGGTCC | | | | | | | | | |
| | AGC (SEQ ID NO: 99) | | | | | | | | | |
| 16 | TCAAATTGACTAGTTTTGACT | MQCQAHGILQQ | GGACAAAGCC | GQSLEQPSEVT | ACAT | TSGFY | AATG | NALD | GCTG | CAVN |
| | TTGCCTTATGTTCCCATTTGT | MWGAFLLYVS | TTGAGCAGCCC | AVEGAIVQINC | CTGG | G | CTCT | GL | TGAA | NAR |
| | TTTCTCTGTTCTTTACATGTT | MKMGGTAGQS | TCTGAAGTGAC | TYQTSGFYGLS | GTTTT | (SEQ | GGAT | (SEQ | TAAC | LMF |
| | CGATGTTCACCATAATCACT | LEQPSEVTAVE | AGCTGTGGAA | WYQQHDGGAP | ATGG | ID NO: | GGTT | ID NO: | AATG | (SEQ |
| | TGGATTAAAATGTGTGGATT | GAIVQINCTYQ | GGAGCCATTGT | TFLSYNALDGL | G | 108) | TG | 110) | CCAG | ID NO: |
| | AGTTTTTGGAGATAGGGACC | TSGFYGLSWYQ | CCAGATAAACT | EETGRFSSFLSR | (SEQ | | (SEQ | | ACTC | 112) |
| | TCACCATGTTGCTTAGGCTG | QHDGGAPTFLS | GCACGTACCA | SDSYGYLLLQE | ID NO: | | ID NO: | | ATG | |
| | GTCTCCAGTTCCTGGCCTCA | YNALDGLEETG | GACATCTGGGT | LQMKDSASYFC | 107) | | 109) | | (SEQ | |
| | AGGGATTCTTCTACCTCAGC | RFSSFLSRSDSY | TTTATGGGCTG | AVNNNARLMF | | | | | ID NO: | |
| | GTCTTGAGTAGCTGGGATTA | GYLLLQELQMK | TCCTGGTACCA | GDGTQLVVKP | | | | | 111) | |
| | CAGGCATAAGCCACTGTGCC | DSASYFCAVNN | GCAACATGAT | (SEQ ID NO: | | | | | | |
| | CAGCTTAAAACCTGTGGATT | NARLMFGDGT | GGCGGAGCAC | 524) | | | | | | |
| | TATCAGTAGAAAATGTTCAT | QLVVKPNIQNP | CCACATTTCTT | | | | | | | |
| | GTAAAGATACTCCTGTAAGA | DPAVYQLRDS | TCTTACAATGC | | | | | | | |
| | GAAACCATAGCTGCTCCAGT | KSSDKSVCLFT | TCTGGATGGTT | | | | | | | |
| | GGAAGGAAGCTTAAACTCAT | DFDSQTNVSQS | TGGAGGAGAC | | | | | | | |
| | CCCTTCAAGAAAGAAGCTCC | KDSDVYITDKT | AGGTCGTTTTT | | | | | | | |
| | TCCCTTTGTATTTCTACTGGG | VLDMRSMDFK | CTTCATTCCTT | | | | | | | |
| | TTTTGCATCCGGACTGATCTT | SNSAVAWSNK | AGTCGCTCTGA | | | | | | | |
| | CCTTCCCTCACCCACATGAA | SDFACANAFNN | TAGTTATGGTT | | | | | | | |
| | GTGTCTACCTTCTGCAGACT | SIIPEDTFFPSP | ACCTCCTTCTA | | | | | | | |
| | ACAGTGGCTCAGGAACCGGG | ESSCDVKLVEK | CAGGAGCTCC | | | | | | | |
| | GATGCAGTGCCAGGCTCATG | SFETDTNLNFQ | AGATGAAAGA | | | | | | | |
| | GTATCCTGCAGCAGATGTGG | NLSVIGFRILL | CTCTGCCTCTT | | | | | | | |
| | GGAGCTTTCCTTCTCTATGTT | KVAGFNLLMT | ACTTCTGCGCT | | | | | | | |
| | TCCATGAAGATGGGAGGCAC | LRLWSS | GTGAATAACA | | | | | | | |
| | TGCAGGACAAAGCCTTGAGC | (SEQ ID NO: | ATGCCAGACTC | | | | | | | |
| | AGCCCTCTGAAGTGACAGCT | 522) | ATGTTTGGAGA | | | | | | | |
| | GTGGAAGGAGCCATTGTCCA | | TGGAACTCAGC | | | | | | | |
| | GATAAACTGCACGTACCAGA | | TGGTGGTGAA | | | | | | | |
| | CATCTGGGTTTTATGGGCTGT | | GCCCA | | | | | | | |
| | CCTGGTACCAGCAACATGAT | | (SEQ ID NO: | | | | | | | |
| | GGCGGAGCACCCACATTTCT | | 523) | | | | | | | |
| | TTCTTACAATGCTCTGGATG | | | | | | | | | |
| | GTTTGGAGGAGACAGGTCGT | | | | | | | | | |
| | TTTTCTTCATTCCTTAGTCGC | | | | | | | | | |
| | TCTGATAGTTATGGTTACCTC | | | | | | | | | |
| | CTTCTACAGGAGCTCCAGAT | | | | | | | | | |
| | GAAAGACTCTGCCTCTTACT | | | | | | | | | |
| | TCTGCGCTGTGAATAACAAT | | | | | | | | | |
| | GCCAGACTCATGTTTGGAGA | | | | | | | | | |
| | TGGAACTCAGCTGGTGGTGA | | | | | | | | | |
| | AGCCCAATATCCAGAACCC | | | | | | | | | |
| | TGACCCTGCCGTGTACCAG | | | | | | | | | |
| | CTGAGAGACTCTAAATCCA | | | | | | | | | |
| | GTGACAAGTCTGTCTGCCT | | | | | | | | | |
| | ATTCACCGATTTTGATTCT | | | | | | | | | |
| | CAAACAAATGTGTCACAAA | | | | | | | | | |
| | GTAAGGATTCTGATGTGTA | | | | | | | | | |
| | TATCACAGACAAAACTGTG | | | | | | | | | |
| | CTAGACATGAGGTCTATGG | | | | | | | | | |
| | ACTTCAAGAGCAACAGTGC | | | | | | | | | |
| | TGTGGCCTGGAGCAACAAA | | | | | | | | | |
| | TCTGACTTTGCATGTGCAA | | | | | | | | | |
| | ACGCCTTCAACAACAGCAT | | | | | | | | | |
| | TATTCCAGAAGACACCTTC | | | | | | | | | |
| | TTCCCCAGCCCAGAAAGTT | | | | | | | | | |
| | CCTGTGATGTCAAGCTGGT | | | | | | | | | |
| | CGAGAAAAGCTTTGAAACA | | | | | | | | | |
| | GATACGAACCTAAACTTTC | | | | | | | | | |
| | AAAACCTGTCAGTGATTGG | | | | | | | | | |
| | GTTCCGAATCCTCCTCCTG | | | | | | | | | |
| | AAAGTGGCCGGGTTTAATC | | | | | | | | | |
| | TGCTCATGACGCTGCGGCT | | | | | | | | | |
| | GTGGTCCAGC (SEQ ID NO: | | | | | | | | | |
| | 106) | | | | | | | | | |
| 17 | TGTCTTGAGAAGAGAAACAC | MAQELGMQCQ | GGACAAAACA | GQNIDQPTEMT | ACAT | TSGFN | AATG | NVLD | GCTG | CAVR |
| | TTATGGGGAAATTGAATACT | ARGILQQMWG | TTGACCAGCCC | ATEGAIVQINCT | CTGG | G | TTCTG | GL | TGAG | DPGN |
| | TTTTTTTTTTTTTTTTTTTGAG | VFLLYVSMKM | ACTGAGATGA | YQTSGFNGLFW | GTTC | (SEQ | GATG | (SEQ | AGAT | DMRF |
| | ACGGAGTCTCACTTTGTCGC | GGTTGQNIDQP | CAGCTACGGA | YQQHAGEAPTF | AACG | ID NO: | GTTT | ID NO: | CCGG | (SEQ |
| | CCAGGCTGGAGTGCAGTGGC | TEMTATEGAIV | AGGTGCCATTG | LSYNVLDGLEE | GG | 115) | G | 117) | GCAA | ID NO: |
| | GCGATCTCTGCTCACTGCAA | QINCTYQTSGF | TCCAGATCAAC | KGRFSSFLSRSK | (SEQ | | (SEQ | | TGAC | 119) |
| | ACTCCGCCTCCCGGGTTCCC | NGLFWYQQHA | TGCACGTACCA | GYSYLLLKELQ | ID NO: | | ID NO: | | ATGC | |
| | GCCATTCTCCTGCCTCAGCCT | GEAPTFLSYNV | GACATCTGGGT | MKDSASYLCA | 114) | | 116) | | GC | |
| | CTCGAGTAGCTGGGACTACA | LDGLEEKGRFS | TCAACGGGCTG | VRDPGNDMRF | | | | | (SEQ | |
| | GGCGCCCGCCACAGCGCCCA | SFLSRSKGYSYL | TTCTGGTACCA | GAGTRLTVKP | | | | | ID NO: | |
| | GCTAATTTTTTTGTATTTTTT | LLKELQMKDSA | GCAACATGCTG | (SEQ ID NO: | | | | | 118) | |
| | GGTAGAGACGGGGTTTCACC | SYLCAVRDPGN | GCGAAGCACC | 527) | | | | | | |
| | GTGTTAGCCAGGATGGTCTC | DMRFGAGTRLT | CACATTTCTGT | | | | | | | |
| | GATCTCCTGACCTCGTGATC | VKPNIQNPDPA | CTTACAATGTT | | | | | | | |
| | CGCCCACCTCGGCCTCCCAA | VYQLRDSKSSD | CTGGATGGTTT | | | | | | | |
| | AGCGCTGGGATTAAAGGCGT | KSVCLFTDFDS | GGAGGAGAAA | | | | | | | |
| | GAGCCACCGCGCCCGGCCTA | QTNVSQSKDSD | GGTCGTTTTTC | | | | | | | |
| | CTTTGTCTTATGTTATTCCCA | VYITDKTVLD | TTCATTCCTTA | | | | | | | |
| | TTTGCCGTCTCTGTTCCTTAT | MRSMDFKSNS | GTCGGTCTAAA | | | | | | | |
| | ACATTATGCTTTTTCAACTTT | AVAWSNKSDF | GGGTACAGTTA | | | | | | | |
| | ACCAGAATCACTTGGATTAA | ACANAFNNSII | CCTCCTTTTGA | | | | | | | |
| | AACCCGTGGATTTCTCAGTA | PEDTFFPSPESS | AGGAGCTCCA | | | | | | | |
| | GGAAATGTTCATGTGAAGAC | CDVKLVEKSF | GATGAAAGAC | | | | | | | |
| | ACTTCTGTAGTAACAGAACC | ETDTNLNFQN | TCTGCCTCTTA | | | | | | | |
| | TACAGCTGCTCCTGTAGAAG | LSVIGFRILLL | CCTCTGTGCTG | | | | | | | |
| | GAAGTTGAAAGTCATCCCTT | KVAGFNLLMT | TGAGAGATCC | | | | | | | |
| | CAAGAAAGGGGCTCCTCCCC | LRLWSS | GGGCAATGAC | | | | | | | |
| | TTGTAATTCTACTGGGTTTTG | (SEQ ID NO: | ATGCGCTTTGG | | | | | | | |
| | CATCCAGACTGAGTTTCCTTC | 525) | AGCAGGGACC | | | | | | | |
| | CCTCACCCACATGAAGTGTC | | AGACTGACAG | | | | | | | |
| | TACCTTCTGCAGACTCCAAT | | TAAAACCAA | | | | | | | |
| | GGCTCAGGAACTGGGAATGC | | (SEQ ID NO: | | | | | | | |
| | AGTGCCAGGCTCGTGGTATC | | 526) | | | | | | | |
| | CTGCAGCAGATGTGGGGAGT | | | | | | | | | |
| | TTTCCTTCTTTATGTTTCCAT | | | | | | | | | |
| | GAAGATGGGAGGCACTACA | | | | | | | | | |
| | GGACAAAACATTGACCAGCC | | | | | | | | | |
| | CACTGAGATGACAGCTACGG | | | | | | | | | |
| | AAGGTGCCATTGTCCAGATC | | | | | | | | | |
| | AACTGCACGTACCAGACATC | | | | | | | | | |
| | TGGGTTCAACGGGCTGTTCT | | | | | | | | | |
| | GGTACCAGCAACATGCTGGC | | | | | | | | | |
| | GAAGCACCCACATTTCTGTC | | | | | | | | | |
| | TTACAATGTTCTGGATGGTTT | | | | | | | | | |
| | GGAGGAGAAAGGTCGTTTTT | | | | | | | | | |
| | CTTCATTCCTTAGTCGGTCTA | | | | | | | | | |
| | AAGGGTACAGTTACCTCCTT | | | | | | | | | |
| | TTGAAGGAGCTCCAGATGAA | | | | | | | | | |
| | AGACTCTGCCTCTTACCTCTG | | | | | | | | | |
| | TGCTGTGAGAGATCCGGGCA | | | | | | | | | |
| | ATGACATGCGCTTTGGAGCA | | | | | | | | | |
| | GGGACCAGACTGACAGTAAA | | | | | | | | | |
| | ACCAAATATCCAGAACCCT | | | | | | | | | |
| | GACCCTGCCGTGTACCAGC | | | | | | | | | |
| | TGAGAGACTCTAAATCCAG | | | | | | | | | |
| | TGACAAGTCTGTCTGCCTA | | | | | | | | | |
| | TTCACCGATTTTGATTCTC | | | | | | | | | |
| | AAACAAATGTGTCACAAAG | | | | | | | | | |
| | TAAGGATTCTGATGTGTAT | | | | | | | | | |
| | ATCACAGACAAAACTGTGC | | | | | | | | | |
| | TAGACATGAGGTCTATGGA | | | | | | | | | |
| | CTTCAAGAGCAACAGTGCT | | | | | | | | | |
| | GTGGCCTGGAGCAACAAAT | | | | | | | | | |
| | CTGACTTTGCATGTGCAAA | | | | | | | | | |
| | CGCCTTCAACAACAGCATT | | | | | | | | | |
| | ATTCCAGAAGACACCTTCT | | | | | | | | | |
| | TCCCCAGCCCAGAAAGTTC | | | | | | | | | |
| | CTGTGATGTCAAGCTGGTC | | | | | | | | | |
| | GAGAAAAGCTTTGAAACAG | | | | | | | | | |
| | ATACGAACCTAAACTTTCA | | | | | | | | | |
| | AAACCTGTCAGTGATTGGG | | | | | | | | | |
| | TTCCGAATCCTCCTCCTGA | | | | | | | | | |
| | AAGTGGCCGGGTTTAATCT | | | | | | | | | |
| | GCTCATGACGCTGCGGCTG | | | | | | | | | |
| | TGGTCCAGC (SEQ ID NO: | | | | | | | | | |
| | 113) | | | | | | | | | |
| 18 | GGGACCCACCTATCATAGCA | MTRVSLLWAV | GCCCAGACAG | AQTVTQSQPEM | ACCA | TSENN | CAAG | QEAY | GCTTT | CAFG |
| | TTTCCTGCCCTGAAGGAGAA | VVSTCLESGMA | TCACTCAGTCT | SVQEAETVTLS | GTGA | YY | AAGC | KQQN | CGGG | GETSG |
| | TTCTCACCAAGCACAGAGGA | QTVTQSQPEMS | CAACCAGAGA | CTYDTSENNYY | GAAT | (SEQ | TTAT | (SEQ | GGAG | SRLTF |
| | GAACCCATCAGAGCAGGAG | VQEAETVTLSC | TGTCTGTGCAG | LFWYKQPPSRQ | AATT | ID NO: | AAGC | ID NO: | AAAC | (SEQ |
| | ACTTTTCACTCTGCAGGGGA | TYDTSENNYYL | GAGGCAGAGA | MILVIRQEAYK | ATTA | 122) | AACA | 124) | CAGT | ID NO: |
| | GCGCTGTCAGCATGACACGA | FWYKQPPSRQ | CTGTGACCCTG | QQNATENRFSV | T | | GAAT | | GGCT | 126) |
| | GTTAGCTTGCTGTGGGCAGT | MILVIRQEAYK | AGTTGCACATA | NFQKAAKSFSL | (SEQ | | (SEQ | | CTAG | |
| | CGTGGTCTCCACCTGTCTTGA | QQNATENRFSV | TGACACCAGTG | KISDSQLGDTA | ID NO: | | ID NO: | | GTTG | |
| | ATCCGGCATGGCCCAGACAG | NFQKAAKSFSL | AGAATAATTAT | MYFCAFGGETS | 121) | | 123) | | ACC | |
| | TCACTCAGTCTCAACCAGAG | KISDSQLGDTA | TATTTGTTCTG | GSRLTFGEGTQ | | | | | (SEQ | |
| | ATGTCTGTGCAGGAGGCAGA | MYFCAFGGETS | GTACAAGCAG | LTVNP | | | | | ID NO: | |
| | GACTGTGACCCTGAGTTGCA | GSRLTFGEGTQ | CCTCCCAGCAG | (SEQ ID NO: | | | | | 125) | |
| | CATATGACACCAGTGAGAAT | LTVNPDIQNPD | GCAGATGATTC | 530) | | | | | | |
| | AATTATTATTTGTTCTGGTAC | PAVYQLRDSKS | TCGTTATTCGC | | | | | | | |
| | AAGCAGCCTCCCAGCAGGCA | SDKSVCLFTDF | CAAGAAGCTT | | | | | | | |
| | GATGATTCTCGTTATTCGCCA | DSQTNVSQSKD | ATAAGCAACA | | | | | | | |
| | AGAAGCTTATAAGCAACAGA | SDVYITDKTVL | GAATGCAACG | | | | | | | |
| | ATGCAACGGAGAATCGTTTC | DMRSMDFKSN | GAGAATCGTTT | | | | | | | |
| | TCTGTGAACTTCCAGAAAGC | SAVAWSNKSD | CTCTGTGAACT | | | | | | | |
| | AGCCAAATCCTTCAGTCTCA | FACANAFNNSI | TCCAGAAAGC | | | | | | | |
| | AGATCTCAGACTCACAGCTG | IPEDTFFPSPES | AGCCAAATCCT | | | | | | | |
| | GGGGACACTGCGATGTATTT | SCDVKLVEKSF | TCAGTCTCAAG | | | | | | | |
| | CTGTGCTTTCGGGGGAGAAA | ETDTNLNFQN | ATCTCAGACTC | | | | | | | |
| | CCAGTGGCTCTAGGTTGACC | LSVIGFRILLL | ACAGCTGGGG | | | | | | | |
| | TTTGGGGAAGGAACACAGCT | KVAGFNLLMT | GACACTGCGAT | | | | | | | |
| | CACAGTGAATCCTGATATCC | LRLWSS | GTATTTCTGTG | | | | | | | |
| | AGAACCCTGACCCTGCCGT | (SEQ ID NO: | CTTTCGGGGGA | | | | | | | |
| | GTACCAGCTGAGAGACTCT | 528) | GAAACCAGTG | | | | | | | |
| | AAATCCAGTGACAAGTCTG | | GCTCTAGGTTG | | | | | | | |
| | TCTGCCTATTCACCGATTT | | ACCTTTGGGGA | | | | | | | |
| | TGATTCTCAAACAAATGTG | | AGGAACACAG | | | | | | | |
| | TCACAAAGTAAGGATTCTG | | CTCACAGTGAA | | | | | | | |
| | ATGTGTATATCACAGACAA | | TCCTG | | | | | | | |
| | AACTGTGCTAGACATGAGG | | (SEQ ID NO: | | | | | | | |
| | TCTATGGACTTCAAGAGCA | | 529) | | | | | | | |
| | ACAGTGCTGTGGCCTGGAG | | | | | | | | | |
| | CAACAAATCTGACTTTGCA | | | | | | | | | |
| | TGTGCAAACGCCTTCAACA | | | | | | | | | |
| | ACAGCATTATTCCAGAAGA | | | | | | | | | |
| | CACCTTCTTCCCCAGCCCA | | | | | | | | | |
| | GAAAGTTCCTGTGATGTCA | | | | | | | | | |
| | AGCTGGTCGAGAAAAGCTT | | | | | | | | | |
| | TGAAACAGATACGAACCTA | | | | | | | | | |
| | AACTTTCAAAACCTGTCAG | | | | | | | | | |
| | TGATTGGGTTCCGAATCCT | | | | | | | | | |
| | CCTCCTGAAAGTGGCCGGG | | | | | | | | | |
| | TTTAATCTGCTCATGACGC | | | | | | | | | |
| | TGCGGCTGTGGTCCAGC | | | | | | | | | |
| | (SEQ ID NO: 120) | | | | | | | | | |
| 19 | AGGGATGTTTTTCTTATATGG | MRQVARVIVFL | CTTGCTAAGAC | LAKTTQPISMD | AACA | NIATN | GGAT | GYKT | CTCG | CLVG |
| | GGAGTTGCTGCTGGGCTCAT | TLSTLSLAKTTQ | CACCCAGCCCA | SYEGQEVNITCS | TTGCT | DY | ACAA | K | TGGG | DKGS |
| | TGCAGCTCAGACACAGCAAA | PISMDSYEGQE | TCTCCATGGAC | HNNIATNDYIT | ACAA | (SEQ | GACA | (SEQ | TGAC | NYQLI |
| | AGAGCCTAGAACCTGGGTCC | VNITCSHNNIAT | TCATATGAAGG | WYQQFPSQGPR | ATGA | ID NO: | AAA | ID NO: | AAAG | W |
| | TAGTTTGCACCTAGAATATG | NDYITWYQQFP | ACAAGAAGTG | FIIQGYKTKVTN | TTAT | 129) | (SEQ | 131) | GAAG | (SEQ |
| | AGGCAAGTGGCGAGAGTGAT | SQGPRFIIQGYK | AACATAACCTG | EVASLFIPADRK | (SEQ | | ID NO: | | CAAC | ID NO: |
| | CGTGTTCCTGACCCTGAGTA | TKVTNEVASLFI | TAGCCACAAC | SSTLSLPRVSLS | ID NO: | | 130) | | TATC | 133) |
| | CTTTGAGCCTTGCTAAGACC | PADRKSSTLSLP | AACATTGCTAC | DTAVYYCLVG | 128) | | | | AGTT | |
| | ACCCAGCCCATCTCCATGGA | RVSLSDTAVYY | AAATGATTATA | DKGSNYQLIWG | | | | | AATC | |
| | CTCATATGAAGGACAAGAAG | CLVGDKGSNY | TCACGTGGTAC | AGTKLIIKP | | | | | (SEQ | |
| | TGAACATAACCTGTAGCCAC | QLIWGAGTKLII | CAACAGTTTCC | (SEQ ID NO: | | | | | ID NO: | |
| | AACAACATTGCTACAAATGA | KPDIQNPDPAV | CAGCCAAGGA | 533) | | | | | 132) | |
| | TTATATCACGTGGTACCAAC | YQLRDSKSSDK | CCACGATTTAT | | | | | | | |
| | AGTTTCCCAGCCAAGGACCA | SVCLFTDFDSQ | TATTCAAGGAT | | | | | | | |
| | CGATTTATTATTCAAGGATA | TNVSQSKDSDV | ACAAGACAAA | | | | | | | |
| | CAAGACAAAAGTTACAAACG | YITDKTVLDM | AGTTACAAAC | | | | | | | |
| | AAGTGGCCTCCCTGTTTATCC | RSMDFKSNSA | GAAGTGGCCTC | | | | | | | |
| | CTGCCGACAGAAAGTCCAGC | VAWSNKSDFA | CCTGTTTATCC | | | | | | | |
| | ACTCTGAGCCTGCCCCGGGT | CANAFNNSIIPE | CTGCCGACAG | | | | | | | |
| | TTCCCTGAGCGACACTGCTG | DTFFPSPESSC | AAAGTCCAGC | | | | | | | |
| | TGTACTACTGCCTCGTGGGT | DVKLVEKSFET | ACTCTGAGCCT | | | | | | | |
| | GACAAAGGAAGCAACTATCA | DTNLNFQNLSV | GCCCCGGGTTT | | | | | | | |
| | GTTAATCTGGGGCGCTGGGA | IGFRILLLKVA | CCCTGAGCGAC | | | | | | | |
| | CCAAGCTAATTATAAAGCCA | GFNLLMTLRL | ACTGCTGTGTA | | | | | | | |
| | GATATCCAGAACCCTGACC | WSS | CTACTGCCTCG | | | | | | | |
| | CTGCCGTGTACCAGCTGAG | (SEQ ID NO: | TGGGTGACAA | | | | | | | |
| | AGACTCTAAATCCAGTGAC | 531) | AGGAAGCAAC | | | | | | | |
| | AAGTCTGTCTGCCTATTCA | | TATCAGTTAAT | | | | | | | |
| | CCGATTTTGATTCTCAAAC | | CTGGGGCGCTG | | | | | | | |
| | AAATGTGTCACAAAGTAAG | | GGACCAAGCT | | | | | | | |
| | GATTCTGATGTGTATATCA | | AATTATAAAGC | | | | | | | |
| | CAGACAAAACTGTGCTAGA | | CAG | | | | | | | |
| | CATGAGGTCTATGGACTTC | | (SEQ ID NO: | | | | | | | |
| | AAGAGCAACAGTGCTGTGG | | 532) | | | | | | | |
| | CCTGGAGCAACAAATCTGA | | | | | | | | | |
| | CTTTGCATGTGCAAACGCC | | | | | | | | | |
| | TTCAACAACAGCATTATTC | | | | | | | | | |
| | CAGAAGACACCTTCTTCCC | | | | | | | | | |
| | CAGCCCAGAAAGTTCCTGT | | | | | | | | | |
| | GATGTCAAGCTGGTCGAGA | | | | | | | | | |
| | AAAGCTTTGAAACAGATAC | | | | | | | | | |
| | GAACCTAAACTTTCAAAAC | | | | | | | | | |
| | CTGTCAGTGATTGGGTTCC | | | | | | | | | |
| | GAATCCTCCTCCTGAAAGT | | | | | | | | | |
| | GGCCGGGTTTAATCTGCTC | | | | | | | | | |
| | ATGACGCTGCGGCTGTGGT | | | | | | | | | |
| | CCAGC | | | | | | | | | |
| | (SEQ ID NO: 127) | | | | | | | | | |
| 20 | GAAGTTCAGTAGTTAGGGAT | MTSIRAVFIFLW | GGAGAGAATG | GENVEQHPSTL | GACA | DSAS | ATTC | IRSNV | GCAG | CAAS |
| | GTGGCCACAAGATGGCAGTG | LQLDLVNGENV | TGGAGCAGCA | SVQEGDSAVIK | GTGC | NY | GTTC | GE | CAAG | YLPD |
| | CTCCTCTGCTGAGGCAGAAT | EQHPSTLSVQE | TCCTTCAACCC | CTYSDSASNYF | CTCA | SEQ | AAAT | (SEQ | TTATC | MRF |
| | ACAGGGTTCACTTTAGTGTG | GDSAVIKCTYS | TGAGTGTCCAG | PWYKQELGKRP | AACT | ID NO: | GTGG | ID NO: | TCCCT | (SEQ |
| | TCCTGAATGAATAGGTTTAT | DSASNYFPWYK | GAGGGAGACA | QLIIDIRSNVGE | AC | 136) | GCGA | 138) | GACA | ID NO: |
| | GGTAGCAGCAGAGCCTTTTT | QELGKRPQLIID | GCGCTGTTATC | KKDQRIAVTLN | (SEQ | | A | | TGCG | 140) |
| | CTTATTGGTTGGCTACACAG | IRSNVGEKKDQ | AAGTGTACTTA | KTAKHFSLHITE | ID NO: | | (SEQ | | C | |
| | TGTGAGAAACCCCTATGGCT | RIAVTLNKTAK | TTCAGACAGTG | TQPEDSAVYFC | 135) | | ID NO: | | (SEQ | |
| | GCCAGAGGAGAGAAGAGAC | HFSLHITETQPE | CCTCAAACTAC | AASYLPDMRFG | | | 137) | | ID NO: | |
| | AACCTGATGATAGAAGTAAC | DSAVYFCAASY | TTCCCTTGGTA | AGTRLTVKP | | | | | 139) | |
| | TCTTATAACTGGAGGTTGCA | LPDMRFGAGTR | TAAGCAAGAA | (SEQ ID NO: | | | | | | |
| | GGTCAATGACTGATCTTAAT | LTVKPNIQNPD | CTTGGAAAAA | 536) | | | | | | |
| | TGGGAAGAACAAGGATGAC | PAVYQLRDSKS | GACCTCAGCTT | | | | | | | |
| | ATCCATTCGAGCTGTATTTAT | SDKSVCLFTDF | ATTATAGACAT | | | | | | | |
| | ATTCCTGTGGCTGCAGCTGG | DSQTNVSQSKD | TCGTTCAAATG | | | | | | | |
| | ACTTGGTGAATGGAGAGAAT | SDVYITDKTVL | TGGGCGAAAA | | | | | | | |
| | GTGGAGCAGCATCCTTCAAC | DMRSMDFKSN | GAAAGACCAA | | | | | | | |
| | CCTGAGTGTCCAGGAGGGAG | SAVAWSNKSD | CGAATTGCTGT | | | | | | | |
| | ACAGCGCTGTTATCAAGTGT | FACANAFNNSI | TACATTGAACA | | | | | | | |
| | ACTTATTCAGACAGTGCCTC | IPEDTFFPSPES | AGACAGCCAA | | | | | | | |
| | AAACTACTTCCCTTGGTATA | SCDVKLVEKSF | ACATTTCTCCC | | | | | | | |
| | AGCAAGAACTTGGAAAAAG | ETDTNLNFQN | TGCACATCACA | | | | | | | |
| | ACCTCAGCTTATTATAGACA | LSVIGFRILLL | GAGACCCAAC | | | | | | | |
| | TTCGTTCAAATGTGGGCGAA | KVAGFNLLMT | CTGAAGACTCG | | | | | | | |
| | AAGAAAGACCAACGAATTGC | LRLWSS | GCTGTCTACTT | | | | | | | |
| | TGTTACATTGAACAAGACAG | (SEQ ID NO: | CTGTGCAGCAA | | | | | | | |
| | CCAAACATTTCTCCCTGCAC | 534) | GTTATCTCCCT | | | | | | | |
| | ATCACAGAGACCCAACCTGA | | GACATGCGCTT | | | | | | | |
| | AGACTCGGCTGTCTACTTCT | | TGGAGCAGGG | | | | | | | |
| | GTGCAGCAAGTTATCTCCCT | | ACCAGACTGA | | | | | | | |
| | GACATGCGCTTTGGAGCAGG | | CAGTAAAACC | | | | | | | |
| | GACCAGACTGACAGTAAAAC | | AA | | | | | | | |
| | CAAATATCCAGAACCCTGA | | (SEQ ID NO: | | | | | | | |
| | CCCTGCCGTGTACCAGCTG | | 535) | | | | | | | |
| | AGAGACTCTAAATCCAGTG | | | | | | | | | |
| | ACAAGTCTGTCTGCCTATT | | | | | | | | | |
| | CACCGATTTTGATTCTCAA | | | | | | | | | |
| | ACAAATGTGTCACAAAGTA | | | | | | | | | |
| | AGGATTCTGATGTGTATAT | | | | | | | | | |
| | CACAGACAAAACTGTGCTA | | | | | | | | | |
| | GACATGAGGTCTATGGACT | | | | | | | | | |
| | TCAAGAGCAACAGTGCTGT | | | | | | | | | |
| | GGCCTGGAGCAACAAATCT | | | | | | | | | |
| | GACTTTGCATGTGCAAACG | | | | | | | | | |
| | CCTTCAACAACAGCATTAT | | | | | | | | | |
| | TCCAGAAGACACCTTCTTC | | | | | | | | | |
| | CCCAGCCCAGAAAGTTCCT | | | | | | | | | |
| | GTGATGTCAAGCTGGTCGA | | | | | | | | | |
| | GAAAAGCTTTGAAACAGAT | | | | | | | | | |
| | ACGAACCTAAACTTTCAAA | | | | | | | | | |
| | ACCTGTCAGTGATTGGGTT | | | | | | | | | |
| | CCGAATCCTCCTCCTGAAA | | | | | | | | | |
| | GTGGCCGGGTTTAATCTGC | | | | | | | | | |
| | TCATGACGCTGCGGCTGTG | | | | | | | | | |
| | GTCCAGC (SEQ ID NO: 134) | | | | | | | | | |
| 21 | GTCTAGCATCTTCCATCTCTA | MNMLTASLLR | GCTCAGAAGG | AQKVTQAQTEI | ACCC | TRDTT | CGGA | RNSFD | GCTC | CALSE |
| | CAGGAAGTATGTGACATGAC | AVIASICVVSSM | TAACTCAAGCG | SVVEKEDVTLD | GTGA | YY | ACTC | EQN | TGAG | LKAA |
| | ACAGTCAAACTCATCATATA | AQKVTQAQTEI | CAGACTGAAA | CVYETRDTTYY | TACT | (SEQ | TTTTG | (SEQ | TGAG | GNKL |
| | TCTGTTAACTATACTTCCTGT | SVVEKEDVTLD | TTTCTGTGGTG | LFWYKQPPSGE | ACTT | ID NO: | ATGA | ID NO: | CTCA | TF |
| | AAAAGGCAGAAGCCTCACAC | CVYETRDTTYY | GAGAAGGAGG | LVFLIRRNSFDE | ATTA | 143) | GCAA | 145) | AAGC | (SEQ |
| | AGCCCAGTAACTTTGCTAGT | LFWYKQPPSGE | ATGTGACCTTG | QNEISGRYSWN | C | | AAT | | TGCA | ID NO: |
| | ACCTCTTGAGTGCAAGGTGG | LVFLIRRNSFDE | GACTGTGTGTA | FQKSTSSFNFTI | (SEQ | | (SEQ | | GGCA | 147) |
| | AGAATTAAGATCTGGATTTG | QNEISGRYSWN | TGAAACCCGTG | TASQVVDSAVY | ID NO: | | ID NO: | | ACAA | |
| | AGACGGAGCACGGAACATTT | FQKSTSSFNFTI | ATACTACTTAT | FCALSELKAAG | 142) | | 144) | | GCTA | |
| | CACTCAGGGGAAGAGCTATG | TASQVVDSAVY | TACTTATTCTG | NKLTFGGGTRV | | | | | ACT | |
| | AACATGCTGACTGCCAGCCT | FCALSELKAAG | GTACAAGCAA | LVKP | | | | | (SEQ | |
| | GTTGAGGGCAGTCATAGCCT | NKLTFGGGTRV | CCACCAAGTG | (SEQ ID NO: | | | | | ID NO: | |
| | CCATCTGTGTTGTATCCAGC | LVKPNIQNPDP | GAGAATTGGTT | 539) | | | | | 146) | |
| | ATGGCTCAGAAGGTAACTCA | AVYQLRDSKSS | TTCCTTATTCG | | | | | | | |
| | AGCGCAGACTGAAATTTCTG | DKSVCLFTDFD | TCGGAACTCTT | | | | | | | |
| | TGGTGGAGAAGGAGGATGTG | SQTNVSQSKDS | TTGATGAGCAA | | | | | | | |
| | ACCTTGGACTGTGTGTATGA | DVYITDKTVLD | AATGAAATAA | | | | | | | |
| | AACCCGTGATACTACTTATT | MRSMDFKSNS | GTGGTCGGTAT | | | | | | | |
| | ACTTATTCTGGTACAAGCAA | AVAWSNKSDF | TCTTGGAACTT | | | | | | | |
| | CCACCAAGTGGAGAATTGGT | ACANAFNNSII | CCAGAAATCC | | | | | | | |
| | TTTCCTTATTCGTCGGAACTC | PEDTFFPSPESS | ACCAGTTCCTT | | | | | | | |
| | TTTTGATGAGCAAAATGAAA | CDVKLVEKSF | CAACTTCACCA | | | | | | | |
| | TAAGTGGTCGGTATTCTTGG | ETDTNLNFQN | TCACAGCCTCA | | | | | | | |
| | AACTTCCAGAAATCCACCAG | LSVIGFRILLL | CAAGTCGTGG | | | | | | | |
| | TTCCTTCAACTTCACCATCAC | KVAGFNLLMT | ACTCAGCAGTA | | | | | | | |
| | AGCCTCACAAGTCGTGGACT | LRLWSS | TACTTCTGTGC | | | | | | | |
| | CAGCAGTATACTTCTGTGCT | (SEQ ID NO: | TCTGAGTGAGC | | | | | | | |
| | CTGAGTGAGCTCAAAGCTGC | 537) | TCAAAGCTGCA | | | | | | | |
| | AGGCAACAAGCTAACTTTTG | | GGCAACAAGC | | | | | | | |
| | GAGGAGGAACCAGGGTGCT | | TAACTTTTGGA | | | | | | | |
| | AGTTAAACCAAATATCCAGA | | GGAGGAACCA | | | | | | | |
| | ACCCTGACCCTGCCGTGTA | | GGGTGCTAGTT | | | | | | | |
| | CCAGCTGAGAGACTCTAAA | | AAACCAA | | | | | | | |
| | TCCAGTGACAAGTCTGTCT | | (SEQ ID NO: | | | | | | | |
| | GCCTATTCACCGATTTTGA | | 538) | | | | | | | |
| | TTCTCAAACAAATGTGTCA | | | | | | | | | |
| | CAAAGTAAGGATTCTGATG | | | | | | | | | |
| | TGTATATCACAGACAAAAC | | | | | | | | | |
| | TGTGCTAGACATGAGGTCT | | | | | | | | | |
| | ATGGACTTCAAGAGCAACA | | | | | | | | | |
| | GTGCTGTGGCCTGGAGCAA | | | | | | | | | |
| | CAAATCTGACTTTGCATGT | | | | | | | | | |
| | GCAAACGCCTTCAACAACA | | | | | | | | | |
| | GCATTATTCCAGAAGACAC | | | | | | | | | |
| | CTTCTTCCCCAGCCCAGAA | | | | | | | | | |
| | AGTTCCTGTGATGTCAAGC | | | | | | | | | |
| | TGGTCGAGAAAAGCTTTGA | | | | | | | | | |
| | AACAGATACGAACCTAAAC | | | | | | | | | |
| | TTTCAAAACCTGTCAGTGA | | | | | | | | | |
| | TTGGGTTCCGAATCCTCCT | | | | | | | | | |
| | CCTGAAAGTGGCCGGGTTT | | | | | | | | | |
| | AATCTGCTCATGACGCTGC | | | | | | | | | |
| | GGCTGTGGTCCAGC (SEQ | | | | | | | | | |
| | ID NO: 141) | | | | | | | | | |
| 22 | GGCTGTACTGGGGCAGCCTG | MRQVARVIVFL | CTTGCTAAGAC | LAKTTQPISMD | AACA | NIATN | GGAT | GYKT | CTCG | CLVG |
| | AGTGACAGCTGCTGGTGTGG | TLSTLSLAKTTQ | CACCCAGCCCA | SYEGQEVNITCS | TTGCT | DY | ACAA | K | TGGG | DKVY |
| | GCCCTGGCAGTTGCTGCTGG | PISMDSYEGQE | TCTCCATGGAC | HNNIATNDYIT | ACAA | (SEQ | GACA | (SEQ | TGAC | GGSQ |
| | GCTCATTGCAGCTCAGACAC | VNITCSHNNIAT | TCATATGAAGG | WYQQFPSQGPR | ATGA | ID NO: | AAA | ID NO: | AAGG | GNLIF |
| | AGCAAAAGAGCCTAGAACCT | NDYITWYQQFP | ACAAGAAGTG | FIIQGYKTKVTN | TTAT | 150) | (SEQ | 152) | TTTAT | (SEQ |
| | GGGTCCTAGTTTGCACCTAG | SQGPRFIIQGYK | AACATAACCTG | EVASLFIPADRK | (SEQ | | ID NO: | | GGAG | ID NO: |
| | AATATGAGGCAAGTGGCGAG | TKVTNEVASLFI | TAGCCACAAC | SSTLSLPRVSLS | ID NO: | | 151) | | GAAG | 154) |
| | AGTGATCGTGTTCCTGACCC | PADRKSSTLSLP | AACATTGCTAC | DTAVYYCLVG | 149) | | | | CCAA | |
| | TGAGTACTTTGAGCCTTGCT | RVSLSDTAVYY | AAATGATTATA | DKVYGGSQGN | | | | | GGAA | |
| | AAGACCACCCAGCCCATCTC | CLVGDKVYGG | TCACGTGGTAC | LIFGKGTKLSV | | | | | ATCT | |
| | CATGGACTCATATGAAGGAC | SQGNLIFGKGT | CAACAGTTTCC | KP | | | | | CATC | |
| | AAGAAGTGAACATAACCTGT | KLSVKPNIQNP | CAGCCAAGGA | (SEQ ID NO: | | | | | (SEQ | |
| | AGCCACAACAACATTGCTAC | DPAVYQLRDS | CCACGATTTAT | 542) | | | | | ID NO: | |
| | AAATGATTATATCACGTGGT | KSSDKSVCLFT | TATTCAAGGAT | | | | | | 153) | |
| | ACCAACAGTTTCCCAGCCAA | DFDSQTNVSQS | ACAAGACAAA | | | | | | | |
| | GGACCACGATTTATTATTCA | KDSDVYITDKT | AGTTACAAAC | | | | | | | |
| | AGGATACAAGACAAAAGTTA | VLDMRSMDFK | GAAGTGGCCTC | | | | | | | |
| | CAAACGAAGTGGCCTCCCTG | SNSAVAWSNK | CCTGTTTATCC | | | | | | | |
| | TTTATCCCTGCCGACAGAAA | SDFACANAFNN | CTGCCGACAG | | | | | | | |
| | GTCCAGCACTCTGAGCCTGC | SIIPEDTFFPSP | AAAGTCCAGC | | | | | | | |
| | CCCGGGTTTCCCTGAGCGAC | ESSCDVKLVEK | ACTCTGAGCCT | | | | | | | |
| | ACTGCTGTGTACTACTGCCTC | SFETDTNLNFQ | GCCCCGGGTTT | | | | | | | |
| | GTGGGTGACAAGGTTTATGG | NLSVIGFRILLL | CCCTGAGCGAC | | | | | | | |
| | AGGAAGCCAAGGAAATCTCA | KVAGFNLLMT | ACTGCTGTGTA | | | | | | | |
| | TCTTTGGAAAAGGCACTAAA | LRLWSS | CTACTGCCTCG | | | | | | | |
| | CTCTCTGTTAAACCAAATAT | (SEQ ID NO: | TGGGTGACAA | | | | | | | |
| | CCAGAACCCTGACCCTGCC | 540) | GGTTTATGGAG | | | | | | | |
| | GTGTACCAGCTGAGAGACT | | GAAGCCAAGG | | | | | | | |
| | CTAAATCCAGTGACAAGTC | | AAATCTCATCT | | | | | | | |
| | TGTCTGCCTATTCACCGAT | | TTGGAAAAGG | | | | | | | |
| | TTTGATTCTCAAACAAATG | | CACTAAACTCT | | | | | | | |
| | TGTCACAAAGTAAGGATTC | | CTGTTAAACCA | | | | | | | |
| | TGATGTGTATATCACAGAC | | A | | | | | | | |
| | AAAACTGTGCTAGACATGA | | (SEQ ID NO: | | | | | | | |
| | GGTCTATGGACTTCAAGAG | | 541) | | | | | | | |
| | CAACAGTGCTGTGGCCTGG | | | | | | | | | |
| | AGCAACAAATCTGACTTTG | | | | | | | | | |
| | CATGTGCAAACGCCTTCAA | | | | | | | | | |
| | CAACAGCATTATTCCAGAA | | | | | | | | | |
| | GACACCTTCTTCCCCAGCC | | | | | | | | | |
| | CAGAAAGTTCCTGTGATGT | | | | | | | | | |
| | CAAGCTGGTCGAGAAAAGC | | | | | | | | | |
| | TTTGAAACAGATACGAACC | | | | | | | | | |
| | TAAACTTTCAAAACCTGTC | | | | | | | | | |
| | AGTGATTGGGTTCCGAATC | | | | | | | | | |
| | CTCCTCCTGAAAGTGGCCG | | | | | | | | | |
| | GGTTTAATCTGCTCATGAC | | | | | | | | | |
| | GCTGCGGCTGTGGTCCAGC | | | | | | | | | |
| | (SEQ ID NO: 148) | | | | | | | | | |
| 23 | GGGGAGTTGGTTGATCCAAA | MSLSSLLKVVT | GCCCAGAAGA | AQKITQTQPGM | ACCA | TSDPS | CAGG | QGSY | GCAA | CAMR |
| | AGAAAGTCTGCATTGGGTGA | ASLWLGPGIAQ | TAACTCAAACC | FVQEKEAVTLD | GTGA | YG | GGTC | DQQN | TGAG | EGQD |
| | GCTTCTCAGCCAGTACATAG | KITQTQPGMFV | CAACCAGGAA | CTYDTSDPSYG | TCCA | (SEQ | TTAT | (SEQ | AGAG | SSYKL |
| | AGAAGTGTGAGATGACACAC | QEKEAVTLDCT | TGTTCGTGCAG | LFWYKQPSSGE | AGTT | ID NO: | GACC | ID NO: | GGCC | IF |
| | ACTAGTTTGCTATACCAGGA | YDTSDPSYGLF | GAAAAGGAGG | MIFLIYQGSYD | ATGG | 157) | AGCA | 159) | AGGA | (SEQ |
| | AACCTCTGTTTCCTGTTCTAG | WYKQPSSGEMI | CTGTGACTCTG | QQNATEGRYSL | T | | AAAT | | TAGC | ID NO: |
| | GGCAGCAGTAGCAGCAGCAC | FLIYQGSYDQQ | GACTGCACATA | NFQKARKSANL | (SEQ | | (SEQ | | AGCT | 161) |
| | ATGGCCCAGTAATTCTTCTCT | NATEGRYSLNF | TGACACCAGTG | VISASQLGDSA | ID NO: | | ID NO: | | ATAA | |
| | CACCATGCCAGGTTCACTTC | QKARKSANLVI | ATCCAAGTTAT | MYFCAMREGQ | 156) | | 158) | | ATTG | |
| | ACAGTACAGAGTCCTGAAAA | SASQLGDSAMY | GGTCTATTCTG | DSSYKLIFGSGT | | | | | ATC | |
| | TAAAGAAGAAAATTTTTTTT | FCAMREGQDSS | GTACAAGCAG | RLLVRP | | | | | (SEQ | |
| | TATCTAGAAAAAGAACCAAA | YKLIFGSGTRLL | CCCAGCAGTG | (SEQ ID NO: | | | | | ID NO: | |
| | CATGTCACTTTCTAGCCTGCT | VRPDIQNPDPA | GGGAAATGAT | 545) | | | | | 160) | |
| | GAAGGTGGTCACAGCTTCAC | VYQLRDSKSSD | TTTTCTTATTT | | | | | | | |
| | TGTGGCTAGGACCTGGCATT | KSVCLFTDFDS | ATCAGGGGTCT | | | | | | | |
| | GCCCAGAAGATAACTCAAAC | QTNVSQSKDSD | TATGACCAGCA | | | | | | | |
| | CCAACCAGGAATGTTCGTGC | VYITDKTVLD | AAATGCAACA | | | | | | | |
| | AGGAAAAGGAGGCTGTGACT | MRSMDFKSNS | GAAGGTCGCT | | | | | | | |
| | CTGGACTGCACATATGACAC | AVAWSNKSDF | ACTCATTGAAT | | | | | | | |
| | CAGTGATCCAAGTTATGGTC | ACANAFNNSII | TTCCAGAAGGC | | | | | | | |
| | TATTCTGGTACAAGCAGCCC | PEDTFFPSPESS | AAGAAAATCC | | | | | | | |
| | AGCAGTGGGGAAATGATTTT | CDVKLVEKSF | GCCAACCTTGT | | | | | | | |
| | TCTTATTTATCAGGGGTCTTA | ETDTNLNFQN | CATCTCCGCTT | | | | | | | |
| | TGACCAGCAAAATGCAACAG | LSVIGFRILLL | CACAACTGGG | | | | | | | |
| | AAGGTCGCTACTCATTGAAT | KVAGFNLLMT | GGACTCAGCA | | | | | | | |
| | TTCCAGAAGGCAAGAAAATC | LRLWSS | ATGTATTTCTG | | | | | | | |
| | CGCCAACCTTGTCATCTCCG | (SEQ ID NO: | TGCAATGAGA | | | | | | | |
| | CTTCACAACTGGGGGACTCA | 543) | GAGGGCCAGG | | | | | | | |
| | GCAATGTATTTCTGTGCAAT | | ATAGCAGCTAT | | | | | | | |
| | GAGAGAGGGCCAGGATAGC | | AAATTGATCTT | | | | | | | |
| | AGCTATAAATTGATCTTCGG | | CGGGAGTGGG | | | | | | | |
| | GAGTGGGACCAGACTGCTGG | | ACCAGACTGCT | | | | | | | |
| | TCAGGCCTGATATCCAGAAC | | GGTCAGGCCTG | | | | | | | |
| | CCTGACCCTGCCGTGTACC | | (SEQ ID NO: | | | | | | | |
| | AGCTGAGAGACTCTAAATC | | 544) | | | | | | | |
| | CAGTGACAAGTCTGTCTGC | | | | | | | | | |
| | CTATTCACCGATTTTGATT | | | | | | | | | |
| | CTCAAACAAATGTGTCACA | | | | | | | | | |
| | AAGTAAGGATTCTGATGTG | | | | | | | | | |
| | TATATCACAGACAAAACTG | | | | | | | | | |
| | TGCTAGACATGAGGTCTAT | | | | | | | | | |
| | GGACTTCAAGAGCAACAGT | | | | | | | | | |
| | GCTGTGGCCTGGAGCAACA | | | | | | | | | |
| | AATCTGACTTTGCATGTGC | | | | | | | | | |
| | AAACGCCTTCAACAACAGC | | | | | | | | | |
| | ATTATTCCAGAAGACACCT | | | | | | | | | |
| | TCTTCCCCAGCCCAGAAAG | | | | | | | | | |
| | TTCCTGTGATGTCAAGCTG | | | | | | | | | |
| | GTCGAGAAAAGCTTTGAAA | | | | | | | | | |
| | CAGATACGAACCTAAACTT | | | | | | | | | |
| | TCAAAACCTGTCAGTGATT | | | | | | | | | |
| | GGGTTCCGAATCCTCCTCC | | | | | | | | | |
| | TGAAAGTGGCCGGGTTTAA | | | | | | | | | |
| | TCTGCTCATGACGCTGCGG | | | | | | | | | |
| | CTGTGGTCCAGC (SEQ ID | | | | | | | | | |
| | NO: 155) | | | | | | | | | |
| 24 | TCTGGGGTTCATATGTAAAA | MAMLLGASVLI | GACCAGCAAG | DQQVKQNSPSL | AACA | NSMF | ATAA | ISSIKD | GCAG | CAARI |
| | TGAAGGGTCTGTGGAAGGAC | LWLQPDWVNS | TTAAGCAAAAT | SVQEGRISILNC | GCAT | DY | GTTC | K | CAAG | YGGS |
| | ATGAATAAAGCACAGGAGGT | QQKNDDQQVK | TCACCATCCCT | DYTNSMFDYFL | GTTT | (SEQ | CATT | (SEQ | GATT | QGNLI |
| | TGAAGTCAGATTTGCAGCTT | QNSPSLSVQEG | GAGCGTCCAG | WYKKYPAEGP | GATT | ID NO: | AAGG | ID NO: | TATG | F |
| | TCTAGGCAGGAGACAAGACA | RISILNCDYTNS | GAAGGAAGAA | TFLISISSIKDKN | AT | 164) | ATAA | 166) | GAGG | (SEQ |
| | ATCTGCATCTTCACAGGAGG | MFDYFLWYKK | TTTCTATTCTG | EDGRFTVFLNK | (SEQ | | A | | AAGC | ID NO: |
| | GATGGCCATGCTCCTGGGGG | YPAEGPTFLISIS | AACTGTGACTA | SAKHLSLHIVPS | ID NO: | | (SEQ | | CAAG | 168) |
| | CATCAGTGCTGATTCTGTGG | SIKDKNEDGRF | TACTAACAGCA | QPGDSAVYFCA | 163) | | ID NO: | | GAAA | |
| | CTTCAGCCAGACTGGGTAAA | TVFLNKSAKHL | TGTTTGATTAT | ARIYGGSQGNLI | | | 165) | | TCTC | |
| | CAGTCAACAGAAGAATGATG | SLHIVPSQPGDS | TTCCTATGGTA | FGKGTKLSVKP | | | | | ATC | |
| | ACCAGCAAGTTAAGCAAAAT | AVYFCAARIYG | CAAAAAATAC | (SEQ ID NO: | | | | | (SEQ | |
| | TCACCATCCCTGAGCGTCCA | GSQGNLIFGKG | CCTGCTGAAGG | 548) | | | | | ID NO: | |
| | GGAAGGAAGAATTTCTATTC | TKLSVKPNIQN | TCCTACATTCC | | | | | | 167) | |
| | TGAACTGTGACTATACTAAC | PDPAVYQLRDS | TGATATCTATA | | | | | | | |
| | AGCATGTTTGATTATTTCCTA | KSSDKSVCLFT | AGTTCCATTAA | | | | | | | |
| | TGGTACAAAAAATACCCTGC | DFDSQTNVSQS | GGATAAAAAT | | | | | | | |
| | TGAAGGTCCTACATTCCTGA | KDSDVYITDKT | GAAGATGGAA | | | | | | | |
| | TATCTATAAGTTCCATTAAG | VLDMRSMDFK | GATTCACTGTC | | | | | | | |
| | GATAAAAATGAAGATGGAA | SNSAVAWSNK | TTCTTAAACAA | | | | | | | |
| | GATTCACTGTCTTCTTAAACA | SDFACANAFNN | AAGTGCCAAG | | | | | | | |
| | AAAGTGCCAAGCACCTCTCT | SIIPEDTFFPSP | CACCTCTCTCT | | | | | | | |
| | CTGCACATTGTGCCCTCCCA | ESSCDVKLVEK | GCACATTGTGC | | | | | | | |
| | GCCTGGAGACTCTGCAGTGT | SFETDTNLNFQ | CCTCCCAGCCT | | | | | | | |
| | ACTTCTGTGCAGCAAGGATT | NLSVIGFRILLL | GGAGACTCTGC | | | | | | | |
| | TATGGAGGAAGCCAAGGAA | KVAGFNLLMT | AGTGTACTTCT | | | | | | | |
| | ATCTCATCTTTGGAAAAGGC | LRLWSS | GTGCAGCAAG | | | | | | | |
| | ACTAAACTCTCTGTTAAACC | (SEQ ID NO: | GATTTATGGAG | | | | | | | |
| | AAATATCCAGAACCCTGAC | 546) | GAAGCCAAGG | | | | | | | |
| | CCTGCCGTGTACCAGCTGA | | AAATCTCATCT | | | | | | | |
| | GAGACTCTAAATCCAGTGA | | TTGGAAAAGG | | | | | | | |
| | CAAGTCTGTCTGCCTATTC | | CACTAAACTCT | | | | | | | |
| | ACCGATTTTGATTCTCAAA | | CTGTTAAACCA | | | | | | | |
| | CAAATGTGTCACAAAGTAA | | A | | | | | | | |
| | GGATTCTGATGTGTATATC | | (SEQ ID NO: | | | | | | | |
| | ACAGACAAAACTGTGCTAG | | 547) | | | | | | | |
| | ACATGAGGTCTATGGACTT | | | | | | | | | |
| | CAAGAGCAACAGTGCTGTG | | | | | | | | | |
| | GCCTGGAGCAACAAATCTG | | | | | | | | | |
| | ACTTTGCATGTGCAAACGC | | | | | | | | | |
| | CTTCAACAACAGCATTATT | | | | | | | | | |
| | CCAGAAGACACCTTCTTCC | | | | | | | | | |
| | CCAGCCCAGAAAGTTCCTG | | | | | | | | | |
| | TGATGTCAAGCTGGTCGAG | | | | | | | | | |
| | AAAAGCTTTGAAACAGATA | | | | | | | | | |
| | CGAACCTAAACTTTCAAAA | | | | | | | | | |
| | CCTGTCAGTGATTGGGTTC | | | | | | | | | |
| | CGAATCCTCCTCCTGAAAG | | | | | | | | | |
| | TGGCCGGGTTTAATCTGCT | | | | | | | | | |
| | CATGACGCTGCGGCTGTGG | | | | | | | | | |
| | TCCAGC | | | | | | | | | |
| | (SEQ ID NO: 162) | | | | | | | | | |
| 25 | GAAAGTCTGCATTGGGTGAG | MSLSSLLKVVT | GCCCAGAAGA | AQKITQTQPGM | ACCA | TSDPS | CAGG | QGSY | GCAA | CAMR |
| | CTTCTCAGCCAGTACATAGA | ASLWLGPGIAQ | TAACTCAAACC | FVQEKEAVTLD | GTGA | YG | GGTC | DQQN | TGAG | EGEPS |
| | GAAGTGTGAGATGACACACA | KITQTQPGMFV | CAACCAGGAA | CTYDTSDPSYG | TCCA | (SEQ | TTAT | (SEQ | AGAG | GSAR |
| | CTAGTTTGCTATACCAGGAA | QEKEAVTLDCT | TGTTCGTGCAG | LFWYKQPSSGE | AGTT | ID NO: | GACC | ID NO: | GGCG | QLTF |
| | ACCTCTGTTTCCTGTTCTAGG | YDTSDPSYGLF | GAAAAGGAGG | MIFLIYQGSYD | ATGG | 171) | AGCA | 173) | AACC | (SEQ |
| | GCAGCAGTAGCAGCAGCACA | WYKQPSSGEMI | CTGTGACTCTG | QQNATEGRYSL | T | | AAAT | | TTCTG | ID NO: |
| | TGGCCCAGTAATTCTTCTCTC | FLIYQGSYDQQ | GACTGCACATA | NFQKARKSANL | (SEQ | | (SEQ | | GTTCT | 175) |
| | ACCATGCCAGGTTCACTTCA | NATEGRYSLNF | TGACACCAGTG | VISASQLGDSA | ID NO: | | ID NO: | | GCAA | |
| | CAGTACAGAGTCCTGAAAAT | QKARKSANLVI | ATCCAAGTTAT | MYFCAMREGE | 170) | | 172) | | GGCA | |
| | AAAGAAGAAAATTTTTTTTT | SASQLGDSAMY | GGTCTATTCTG | PSGSARQLTFGS | | | | | ACTG | |
| | ATCTAGAAAAAGAACCAAAC | FCAMREGEPSG | GTACAAGCAG | GTQLTVLP | | | | | ACC | |
| | ATGTCACTTTCTAGCCTGCTG | SARQLTFGSGT | CCCAGCAGTG | (SEQ ID NO: | | | | | (SEQ | |
| | AAGGTGGTCACAGCTTCACT | QLTVLPDIQNP | GGGAAATGAT | 551) | | | | | ID NO: | |
| | GTGGCTAGGACCTGGCATTG | DPAVYQLRDS | TTTTCTTATTT | | | | | | 174) | |
| | CCCAGAAGATAACTCAAACC | KSSDKSVCLFT | ATCAGGGGTCT | | | | | | | |
| | CAACCAGGAATGTTCGTGCA | DFDSQTNVSQS | TATGACCAGCA | | | | | | | |
| | GGAAAAGGAGGCTGTGACTC | KDSDVYITDKT | AAATGCAACA | | | | | | | |
| | TGGACTGCACATATGACACC | VLDMRSMDFK | GAAGGTCGCT | | | | | | | |
| | AGTGATCCAAGTTATGGTCT | SNSAVAWSNK | ACTCATTGAAT | | | | | | | |
| | ATTCTGGTACAAGCAGCCCA | SDFACANAFNN | TTCCAGAAGGC | | | | | | | |
| | GCAGTGGGGAAATGATTTTT | SIIPEDTFFPSP | AAGAAAATCC | | | | | | | |
| | CTTATTTATCAGGGGTCTTAT | ESSCDVKLVEK | GCCAACCTTGT | | | | | | | |
| | GACCAGCAAAATGCAACAG | SFETDTNLNFQ | CATCTCCGCTT | | | | | | | |
| | AAGGTCGCTACTCATTGAAT | NLSVIGFRILLL | CACAACTGGG | | | | | | | |
| | TTCCAGAAGGCAAGAAAATC | KVAGFNLLMT | GGACTCAGCA | | | | | | | |
| | CGCCAACCTTGTCATCTCCG | LRLWSS | ATGTACTTCTG | | | | | | | |
| | CTTCACAACTGGGGGACTCA | (SEQ ID NO: | TGCAATGAGA | | | | | | | |
| | GCAATGTACTTCTGTGCAAT | 549) | GAGGGCGAAC | | | | | | | |
| | GAGAGAGGGCGAACCTTCTG | | CTTCTGGTTCT | | | | | | | |
| | GTTCTGCAAGGCAACTGACC | | GCAAGGCAAC | | | | | | | |
| | TTTGGATCTGGGACACAATT | | TGACCTTTGGA | | | | | | | |
| | GACTGTTTTACCTGATATCC | | TCTGGGACACA | | | | | | | |
| | AGAACCCTGACCCTGCCGT | | ATTGACTGTTT | | | | | | | |
| | GTACCAGCTGAGAGACTCT | | TACCTG | | | | | | | |
| | AAATCCAGTGACAAGTCTG | | (SEQ ID NO: | | | | | | | |
| | TCTGCCTATTCACCGATTT | | 550) | | | | | | | |
| | TGATTCTCAAACAAATGTG | | | | | | | | | |
| | TCACAAAGTAAGGATTCTG | | | | | | | | | |
| | ATGTGTATATCACAGACAA | | | | | | | | | |
| | AACTGTGCTAGACATGAGG | | | | | | | | | |
| | TCTATGGACTTCAAGAGCA | | | | | | | | | |
| | ACAGTGCTGTGGCCTGGAG | | | | | | | | | |
| | CAACAAATCTGACTTTGCA | | | | | | | | | |
| | TGTGCAAACGCCTTCAACA | | | | | | | | | |
| | ACAGCATTATTCCAGAAGA | | | | | | | | | |
| | CACCTTCTTCCCCAGCCCA | | | | | | | | | |
| | GAAAGTTCCTGTGATGTCA | | | | | | | | | |
| | AGCTGGTCGAGAAAAGCTT | | | | | | | | | |
| | TGAAACAGATACGAACCTA | | | | | | | | | |
| | AACTTTCAAAACCTGTCAG | | | | | | | | | |
| | TGATTGGGTTCCGAATCCT | | | | | | | | | |
| | CCTCCTGAAAGTGGCCGGG | | | | | | | | | |
| | TTTAATCTGCTCATGACGC | | | | | | | | | |
| | TGCGGCTGTGGTCCAGC | | | | | | | | | |
| | (SEQ ID NO: 169) | | | | | | | | | |
| 26 | TGGGGTTCATATGTAAAATG | MAMLLGASVLI | GACCAGCAAG | DQQVKQNSPSL | AACA | NSMF | ATAA | ISSIKD | GCAG | CAVN |
| | AAGGGTCTGTGGAAGGACAT | LWLQPDWVNS | TTAAGCAAAAT | SVQEGRISILNC | GCAT | DY | GTTC | K | TTAA | AGGT |
| | GAATAAAGCACAGGAGGTTG | QQKNDDQQVK | TCACCATCCCT | DYTNSMFDYFL | GTTT | (SEQ | CATT | (SEQ | TGCT | SYGK |
| | AAGTCAGATTTGCAGCTTTC | QNSPSLSVQEG | GAGCGTCCAG | WYKKYPAEGP | GATT | ID NO: | AAGG | ID NO: | GGTG | LTF |
| | TAGGCAGGAGACAAGACAA | RISILNCDYTNS | GAAGGAAGAA | TFLISISSIKDKN | AT | 178) | ATAA | 180) | GTAC | (SEQ |
| | TCTGCATCTTCACAGGAGGG | MFDYFLWYKK | TTTCTATTCTG | EDGRFTVFLNK | SEQ | | A | | TAGC | ID NO: |
| | ATGGCCATGCTCCTGGGGGC | YPAEGPTFLISIS | AACTGTGACTA | SAKHLSLHIVPS | ID NO: | | (SEQ | | TATG | 182) |
| | ATCAGTGCTGATTCTGTGGC | SIKDKNEDGRF | TACTAACAGCA | QPGDSAVYFCA | 177) | | ID NO: | | GAAA | |
| | TTCAGCCAGACTGGGTAAAC | TVFLNKSAKHL | TGTTTGATTAT | VNAGGTSYGKL | | | 179) | | GCTG | |
| | AGTCAACAGAAGAATGATGA | SLHIVPSQPGDS | TTCCTATGGTA | TFGQGTILTVHP | | | | | ACA | |
| | CCAGCAAGTTAAGCAAAATT | AVYFCAVNAG | CAAAAAATAC | (SEQ ID NO: | | | | | (SEQ | |
| | CACCATCCCTGAGCGTCCAG | GTSYGKLTFGQ | CCTGCTGAAGG | 554) | | | | | ID NO: | |
| | GAAGGAAGAATTTCTATTCT | GTILTVHPNIQN | TCCTACATTCC | | | | | | 181) | |
| | GAACTGTGACTATACTAACA | PDPAVYQLRDS | TGATATCTATA | | | | | | | |
| | GCATGTTTGATTATTTCCTAT | KSSDKSVCLFT | AGTTCCATTAA | | | | | | | |
| | GGTACAAAAAATACCCTGCT | DFDSQTNVSQS | GGATAAAAAT | | | | | | | |
| | GAAGGTCCTACATTCCTGAT | KDSDVYITDKT | GAAGATGGAA | | | | | | | |
| | ATCTATAAGTTCCATTAAGG | VLDMRSMDFK | GATTCACTGTC | | | | | | | |
| | ATAAAAATGAAGATGGAAG | SNSAVAWSNK | TTCTTAAACAA | | | | | | | |
| | ATTCACTGTCTTCTTAAACAA | SDFACANAFNN | AAGTGCCAAG | | | | | | | |
| | AAGTGCCAAGCACCTCTCTC | SIIPEDTFFPSP | CACCTCTCTCT | | | | | | | |
| | TGCACATTGTGCCCTCCCAG | ESSCDVKLVEK | GCACATTGTGC | | | | | | | |
| | CCTGGAGACTCTGCAGTGTA | SFETDTNLNFQ | CCTCCCAGCCT | | | | | | | |
| | CTTCTGTGCAGTTAATGCTG | NLSVIGFRILLL | GGAGACTCTGC | | | | | | | |
| | GTGGTACTAGCTATGGAAAG | KVAGFNLLMT | AGTGTACTTCT | | | | | | | |
| | CTGACATTTGGACAAGGGAC | LRLWSS | GTGCAGTTAAT | | | | | | | |
| | CATCTTGACTGTCCATCCAA | (SEQ ID NO: | GCTGGTGGTAC | | | | | | | |
| | ATATCCAGAACCCTGACCC | 552) | TAGCTATGGAA | | | | | | | |
| | TGCCGTGTACCAGCTGAGA | | AGCTGACATTT | | | | | | | |
| | GACTCTAAATCCAGTGACA | | GGACAAGGGA | | | | | | | |
| | AGTCTGTCTGCCTATTCAC | | CCATCTTGACT | | | | | | | |
| | CGATTTTGATTCTCAAACA | | GTCCATCCAA | | | | | | | |
| | AATGTGTCACAAAGTAAGG | | (SEQ ID NO: | | | | | | | |
| | ATTCTGATGTGTATATCAC | | 553) | | | | | | | |
| | AGACAAAACTGTGCTAGAC | | | | | | | | | |
| | ATGAGGTCTATGGACTTCA | | | | | | | | | |
| | AGAGCAACAGTGCTGTGGC | | | | | | | | | |
| | CTGGAGCAACAAATCTGAC | | | | | | | | | |
| | TTTGCATGTGCAAACGCCT | | | | | | | | | |
| | TCAACAACAGCATTATTCC | | | | | | | | | |
| | AGAAGACACCTTCTTCCCC | | | | | | | | | |
| | AGCCCAGAAAGTTCCTGTG | | | | | | | | | |
| | ATGTCAAGCTGGTCGAGAA | | | | | | | | | |
| | AAGCTTTGAAACAGATACG | | | | | | | | | |
| | AACCTAAACTTTCAAAACC | | | | | | | | | |
| | TGTCAGTGATTGGGTTCCG | | | | | | | | | |
| | AATCCTCCTCCTGAAAGTG | | | | | | | | | |
| | GCCGGGTTTAATCTGCTCA | | | | | | | | | |
| | TGACGCTGCGGCTGTGGTC | | | | | | | | | |
| | CAGC | | | | | | | | | |
| | (SEQ ID NO: 176) | | | | | | | | | |
| 27 | GACATTTCTCAAATGAGAAG | MLLLLVPVLEV | GCCCAGTCGGT | AQSVTQLGSHV | TCGT | SSVPP | TACA | YTTG | GCTG | CAVS |
| | CAAACAGTTCACTTCCTTGG | IFTLGGTRAQSV | GACCCAGCTTG | SVSEGALVLLR | CTGTT | Y | CAAC | ATLV | TGAG | EKFSG |
| | ATCCGTGGTTTCGCCTGTGG | TQLGSHVSVSE | GCAGCCACGTC | CNYSSSVPPYLF | CCAC | (SEQ | AGGG | (SEQ | TGAG | GYNK |
| | CCTTCAGGGGGCGACGTTGC | GALVLLRCNYS | TCTGTCTCTGA | WYVQYPNQGL | CATA | ID NO: | GCCA | ID NO: | AAGT | LIF |
| | ACTAAGGAGGCATCTGTGTT | SSVPPYLFWYV | GGGAGCCCTG | QLLLKYTTGAT | T | 185) | CCCT | 187) | TTTCT | SEQ |
| | CATTGCCGACCATCCTCATC | QYPNQGLQLLL | GTTCTGCTGAG | LVKGINGFEAE | (SEQ | | GGTT | | GGTG | ID NO: |
| | CACTGAGCCTCCTCCCTGCA | KYTTGATLVKG | GTGCAACTACT | FKKSETSFHLTK | ID NO: | | (SEQ | | GCTA | 189) |
| | GCTGGCTGATGTAGCTCACT | INGFEAEFKKSE | CATCGTCTGTT | PSAHMSDAAEY | 184) | | ID NO: | | CAAT | |
| | GGTGTCTGTGTAGATAGGGA | TSFHLTKPSAH | CCACCATATCT | FCAVSEKFSGG | | | 186) | | AAGC | |
| | GCTGTGATGAGAACAAGAGG | MSDAAEYFCA | CTTCTGGTATG | YNKLIFGAGTR | | | | | TGAT | |
| | TCAGAACACATCCAGGCTCC | VSEKFSGGYNK | TGCAATACCCC | LAVHP | | | | | T | |
| | TTAAGAGAAAGCCTTTCTTT | LIFGAGTRLAV | AACCAAGGAC | (SEQ ID NO: | | | | | (SEQ | |
| | AACCATTTTTGAAACCCTTC | HPYIQNPDPAV | TCCAGCTTCTC | 557) | | | | | ID NO: | |
| | AAAGGCAGAGACTTGTCCAG | YQLRDSKSSDK | CTGAAGTACAC | | | | | | 188) | |
| | CCTAACCTGCCTGCTGCTCCT | SVCLFTDFDSQ | AACAGGGGCC | | | | | | | |
| | AGCTCCTGAGGCTCAGGGCC | TNVSQSKDSDV | ACCCTGGTTAA | | | | | | | |
| | CTTGGCTTCTGTCCGCTCTGC | YITDKTVLDM | AGGCATCAAC | | | | | | | |
| | TCAGGGCCCTCCAGCGTGGC | RSMDFKSNSA | GGTTTTGAGGC | | | | | | | |
| | CACTGCTCAGCCATGCTCCT | VAWSNKSDFA | TGAATTTAAGA | | | | | | | |
| | GCTGCTCGTCCCAGTGCTCG | CANAFNNSIIPE | AGAGTGAAAC | | | | | | | |
| | AGGTGATTTTTACCCTGGGA | DTFFPSPESSC | CTCCTTCCACC | | | | | | | |
| | GGAACCAGAGCCCAGTCGGT | DVKLVEKSFET | TGACGAAACC | | | | | | | |
| | GACCCAGCTTGGCAGCCACG | DTNLNFQNLSV | CTCAGCCCATA | | | | | | | |
| | TCTCTGTCTCTGAGGGAGCC | IGFRILLLKVA | TGAGCGACGC | | | | | | | |
| | CTGGTTCTGCTGAGGTGCAA | GFNLLMTLRL | GGCTGAGTACT | | | | | | | |
| | CTACTCATCGTCTGTTCCACC | WSS | TCTGTGCTGTG | | | | | | | |
| | ATATCTCTTCTGGTATGTGCA | (SEQ ID NO: | AGTGAGAAGT | | | | | | | |
| | ATACCCCAACCAAGGACTCC | 555) | TTTCTGGTGGC | | | | | | | |
| | AGCTTCTCCTGAAGTACACA | | TACAATAAGCT | | | | | | | |
| | ACAGGGGCCACCCTGGTTAA | | GATTTTTGGAG | | | | | | | |
| | AGGCATCAACGGTTTTGAGG | | CAGGGACCAG | | | | | | | |
| | CTGAATTTAAGAAGAGTGAA | | GCTGGCTGTAC | | | | | | | |
| | ACCTCCTTCCACCTGACGAA | | ACCCAT | | | | | | | |
| | ACCCTCAGCCCATATGAGCG | | (SEQ ID NO: | | | | | | | |
| | ACGCGGCTGAGTACTTCTGT | | 556) | | | | | | | |
| | GCTGTGAGTGAGAAGTTTTC | | | | | | | | | |
| | TGGTGGCTACAATAAGCTGA | | | | | | | | | |
| | TTTTTGGAGCAGGGACCAGG | | | | | | | | | |
| | CTGGCTGTACACCCATATAT | | | | | | | | | |
| | CCAGAACCCTGACCCTGCC | | | | | | | | | |
| | GTGTACCAGCTGAGAGACT | | | | | | | | | |
| | CTAAATCCAGTGACAAGTC | | | | | | | | | |
| | TGTCTGCCTATTCACCGAT | | | | | | | | | |
| | TTTGATTCTCAAACAAATG | | | | | | | | | |
| | TGTCACAAAGTAAGGATTC | | | | | | | | | |
| | TGATGTGTATATCACAGAC | | | | | | | | | |
| | AAAACTGTGCTAGACATGA | | | | | | | | | |
| | GGTCTATGGACTTCAAGAG | | | | | | | | | |
| | CAACAGTGCTGTGGCCTGG | | | | | | | | | |
| | AGCAACAAATCTGACTTTG | | | | | | | | | |
| | CATGTGCAAACGCCTTCAA | | | | | | | | | |
| | CAACAGCATTATTCCAGAA | | | | | | | | | |
| | GACACCTTCTTCCCCAGCC | | | | | | | | | |
| | CAGAAAGTTCCTGTGATGT | | | | | | | | | |
| | CAAGCTGGTCGAGAAAAGC | | | | | | | | | |
| | TTTGAAACAGATACGAACC | | | | | | | | | |
| | TAAACTTTCAAAACCTGTC | | | | | | | | | |
| | AGTGATTGGGTTCCGAATC | | | | | | | | | |
| | CTCCTCCTGAAAGTGGCCG | | | | | | | | | |
| | GGTTTAATCTGCTCATGAC | | | | | | | | | |
| | GCTGCGGCTGTGGTCCAGC | | | | | | | | | |
| | (SEQ ID NO: 183) | | | | | | | | | |
| 28 | TGGGGGTTAACTATACTTCC | MNMLTASLLR | GCTCAGAAGG | AQKVTQAQTEI | ACCC | TRDTT | CGGA | RNSFD | GCTC | CALSE |
| | TGTAAAAGGCAGAAGCCTCA | AVIASICVVSSM | TAACTCAAGCG | SVVEKEDVTLD | GTGA | YY | ACTC | EQN | TGAG | AKDD |
| | CACAGCCCAGTAACTTTGCT | AQKVTQAQTEI | CAGACTGAAA | CVYETRDTTYY | TACT | (SEQ | TTTTG | (SEQ | TGAG | KIIF |
| | AGTACCTCTTGAGTGCAAGG | SVVEKEDVTLD | TTTCTGTGGTG | LFWYKQPPSGE | ACTT | ID NO: | ATGA | ID NO: | GCAA | (SEQ |
| | TGGAGAATTAAGATCTGGAT | CVYETRDTTYY | GAGAAGGAGG | LVFLIRRNSFDE | ATTA | 192) | GCAA | 194) | AAGA | ID NO: |
| | TTGAGACGGAGCACGGAACA | LFWYKQPPSGE | ATGTGACCTTG | QNEISGRYSWN | C | | AAT | | TGAC | 196) |
| | TTTCACTCAGGGGAAGAGCT | LVFLIRRNSFDE | GACTGTGTGTA | FQKSTSSFNFTI | (SEQ | | (SEQ | | AAGA | |
| | ATGAACATGCTGACTGCCAG | QNEISGRYSWN | TGAAACCCGTG | TASQVVDSAVY | ID NO: | | ID NQ | | TCAT | |
| | CCTGTTGAGGGCAGTCATAG | FQKSTSSFNFTI | ATACTACTTAT | FCALSEAKDDK | 191) | | 193) | | C | |
| | CCTCCATCTGTGTTGTATCCA | TASQVVDSAVY | TACTTATTCTG | IIFGKGTRLHILP | | | | | (SEQ | |
| | GCATGGCTCAGAAGGTAACT | FCALSEAKDDK | GTACAAGCAA | (SEQ ID NO: | | | | | ID NO: | |
| | CAAGCGCAGACTGAAATTTC | IIFGKGTRLHILP | CCACCAAGTG | 560) | | | | | 195) | |
| | TGTGGTGGAGAAGGAGGATG | NIQNPDPAVYQ | GAGAATTGGTT | | | | | | | |
| | TGACCTTGGACTGTGTGTAT | LRDSKSSDKSV | TTCCTTATTCG | | | | | | | |
| | GAAACCCGTGATACTACTTA | CLFTDFDSQTN | TCGGAACTCTT | | | | | | | |
| | TTACTTATTCTGGTACAAGC | VSQSKDSDVYI | TTGATGAGCAA | | | | | | | |
| | AACCACCAAGTGGAGAATTG | TDKTVLDMRS | AATGAAATAA | | | | | | | |
| | GTTTTCCTTATTCGTCGGAAC | MDFKSNSAVA | GTGGTCGGTAT | | | | | | | |
| | TCTTTTGATGAGCAAAATGA | WSNKSDFACA | TCTTGGAACTT | | | | | | | |
| | AATAAGTGGTCGGTATTCTT | NAFNNSIIPEDT | CCAGAAATCC | | | | | | | |
| | GGAACTTCCAGAAATCCACC | FFPSPESSCDV | ACCAGTTCCTT | | | | | | | |
| | AGTTCCTTCAACTTCACCATC | KLVEKSFETDT | CAACTTCACCA | | | | | | | |
| | ACAGCCTCACAAGTCGTGGA | NLNFQNLSVIG | TCACAGCCTCA | | | | | | | |
| | CTCAGCAGTATACTTCTGTG | FRILLLKVAGF | CAAGTCGTGG | | | | | | | |
| | CTCTGAGTGAGGCAAAAGAT | NLLMTLRLWS | ACTCAGCAGTA | | | | | | | |
| | GACAAGATCATCTTTGGAAA | S | TACTTCTGTGC | | | | | | | |
| | AGGGACACGACTTCATATTC | (SEQ ID NO: | TCTGAGTGAGG | | | | | | | |
| | TCCCCAATATCCAGAACCCT | 558) | CAAAAGATGA | | | | | | | |
| | GACCCTGCCGTGTACCAGC | | CAAGATCATCT | | | | | | | |
| | TGAGAGACTCTAAATCCAG | | TTGGAAAAGG | | | | | | | |
| | TGACAAGTCTGTCTGCCTA | | GACACGACTTC | | | | | | | |
| | TTCACCGATTTTGATTCTC | | ATATTCTCCCC | | | | | | | |
| | AAACAAATGTGTCACAAAG | | A | | | | | | | |
| | TAAGGATTCTGATGTGTAT | | (SEQ ID NO: | | | | | | | |
| | ATCACAGACAAAACTGTGC | | 559) | | | | | | | |
| | TAGACATGAGGTCTATGGA | | | | | | | | | |
| | CTTCAAGAGCAACAGTGCT | | | | | | | | | |
| | GTGGCCTGGAGCAACAAAT | | | | | | | | | |
| | CTGACTTTGCATGTGCAAA | | | | | | | | | |
| | CGCCTTCAACAACAGCATT | | | | | | | | | |
| | ATTCCAGAAGACACCTTCT | | | | | | | | | |
| | TCCCCAGCCCAGAAAGTTC | | | | | | | | | |
| | CTGTGATGTCAAGCTGGTC | | | | | | | | | |
| | GAGAAAAGCTTTGAAACAG | | | | | | | | | |
| | ATACGAACCTAAACTTTCA | | | | | | | | | |
| | AAACCTGTCAGTGATTGGG | | | | | | | | | |
| | TTCCGAATCCTCCTCCTGA | | | | | | | | | |
| | AAGTGGCCGGGTTTAATCT | | | | | | | | | |
| | GCTCATGACGCTGCGGCTG | | | | | | | | | |
| | TGGTCCAGC | | | | | | | | | |
| | (SEQ ID NO: 190) | | | | | | | | | |
| 29 | CTCCTATATCGGTTTTTCTTA | MAMLLGASVLI | GACCAGCAAG | DQQVKQNSPSL | AACA | NSMF | ATAA | ISSIKD | GCCA | CAIYN |
| | TATGGGAGAATCTCCATGGC | LWLQPDWVNS | TTAAGCAAAAT | SVQEGRISILNC | GCAT | DY | GTTC | K | TTTAT | QGGK |
| | AGCGTTTTCTTATATGGGGTT | QQKNDDQQVK | TCACCATCCCT | DYTNSMFDYFL | GTTT | (SEQ | CATT | (SEQ | AACC | LIF |
| | CATATGTAAAATGAAGGGTC | QNSPSLSVQEG | GAGCGTCCAG | WYKKYPAEGP | GATT | ID NO: | AAGG | ID NO: | AGGG | (SEQ |
| | TGTGGAAGGACATGAATAAA | RISILNCDYTNS | GAAGGAAGAA | TFLISISSIKDKN | AT | 199) | ATAA | 201) | AGGA | ID NO: |
| | GCACAGGAGGTTGAAGTCAG | MFDYFLWYKK | TTTCTATTCTG | EDGRFTVFLNK | (SEQ | | A | | AAGC | 203) |
| | ATTTGCAGCTTTCTAGGCAG | YPAEGPTFLISIS | AACTGTGACTA | SAKHLSLHIVPS | ID NO: | | (SEQ | | TTATC | |
| | GAGACAAGACAATCTGCATC | SIKDKNEDGRF | TACTAACAGCA | QPGDSAVYFCA | 198) | | ID NO: | | (SEQ | |
| | TTCACAGGAGGGATGGCCAT | TVFLNKSAKHL | TGTTTGATTAT | IYNQGGKLIFG | | | 200) | | ID NO: | |
| | GCTCCTGGGGGCATCAGTGC | SLHIVPSQPGDS | TTCCTATGGTA | QGTELSVKP | | | | | 202) | |
| | TGATTCTGTGGCTTCAGCCA | AVYFCAIYNQG | CAAAAAATAC | (SEQ ID NO: | | | | | | |
| | GACTGGGTAAACAGTCAACA | GKLIFGQGTELS | CCTGCTGAAGG | 563) | | | | | | |
| | GAAGAATGATGACCAGCAA | VKPNIQNPDPA | TCCTACATTCC | | | | | | | |
| | GTTAAGCAAAATTCACCATC | VYQLRDSKSSD | TGATATCTATA | | | | | | | |
| | CCTGAGCGTCCAGGAAGGAA | KSVCLFTDFDS | AGTTCCATTAA | | | | | | | |
| | GAATTTCTATTCTGAACTGTG | QTNVSQSKDSD | GGATAAAAAT | | | | | | | |
| | ACTATACTAACAGCATGTTT | VYITDKTVLD | GAAGATGGAA | | | | | | | |
| | GATTATTTCCTATGGTACAA | MRSMDFKSNS | GATTCACTGTC | | | | | | | |
| | AAAATACCCTGCTGAAGGTC | AVAWSNKSDF | TTCTTAAACAA | | | | | | | |
| | CTACATTCCTGATATCTATAA | ACANAFNNSII | AAGTGCCAAG | | | | | | | |
| | GTTCCATTAAGGATAAAAAT | PEDTFFPSPESS | CACCTCTCTCT | | | | | | | |
| | GAAGATGGAAGATTCACTGT | CDVKLVEKSF | GCACATTGTGC | | | | | | | |
| | CTTCTTAAACAAAAGTGCCA | ETDTNLNFQN | CCTCCCAGCCT | | | | | | | |
| | AGCACCTCTCTCTGCACATT | LSVIGFRILLL | GGAGACTCTGC | | | | | | | |
| | GTGCCCTCCCAGCCTGGAGA | KVAGFNLLMT | AGTGTACTTCT | | | | | | | |
| | CTCTGCAGTGTACTTCTGTGC | LRLWSS | GTGCCATTTAT | | | | | | | |
| | CATTTATAACCAGGGAGGAA | (SEQ ID NO: | AACCAGGGAG | | | | | | | |
| | AGCTTATCTTCGGACAGGGA | 561) | GAAAGCTTATC | | | | | | | |
| | ACGGAGTTATCTGTGAAACC | | TTCGGACAGG | | | | | | | |
| | CAATATCCAGAACCCTGAC | | GAACGGAGTT | | | | | | | |
| | CCTGCCGTGTACCAGCTGA | | ATCTGTGAAAC | | | | | | | |
| | GAGACTCTAAATCCAGTGA | | CCA | | | | | | | |
| | CAAGTCTGTCTGCCTATTC | | (SEQ ID NO: | | | | | | | |
| | ACCGATTTTGATTCTCAAA | | 562) | | | | | | | |
| | CAAATGTGTCACAAAGTAA | | | | | | | | | |
| | GGATTCTGATGTGTATATC | | | | | | | | | |
| | ACAGACAAAACTGTGCTAG | | | | | | | | | |
| | ACATGAGGTCTATGGACTT | | | | | | | | | |
| | CAAGAGCAACAGTGCTGTG | | | | | | | | | |
| | GCCTGGAGCAACAAATCTG | | | | | | | | | |
| | ACTTTGCATGTGCAAACGC | | | | | | | | | |
| | CTTCAACAACAGCATTATT | | | | | | | | | |
| | CCAGAAGACACCTTCTTCC | | | | | | | | | |
| | CCAGCCCAGAAAGTTCCTG | | | | | | | | | |
| | TGATGTCAAGCTGGTCGAG | | | | | | | | | |
| | AAAAGCTTTGAAACAGATA | | | | | | | | | |
| | CGAACCTAAACTTTCAAAA | | | | | | | | | |
| | CCTGTCAGTGATTGGGTTC | | | | | | | | | |
| | CGAATCCTCCTCCTGAAAG | | | | | | | | | |
| | TGGCCGGGTTTAATCTGCT | | | | | | | | | |
| | CATGACGCTGCGGCTGTGG | | | | | | | | | |
| | TCCAGC | | | | | | | | | |
| | (SEQ ID NO: 197) | | | | | | | | | |
| 30 | TACATTCTCAATTTCTTATAT | MNYSPGLVSLI | GGAAATTCAGT | GNSVTQMEGPV | GCCA | ATGY | GCCA | ATKA | GCTC | CALM |
| | GGGGACTGTGATTTCTTCAT | LLLLGRTRGNS | GACCCAGATG | TLSEEAFLTINC | CAGG | PS | CGAA | DDK | TGAT | TDYK |
| | GTTAAGGATCAAGACCATTA | VTQMEGPVTLS | GAAGGGCCAG | TYTATGYPSLF | ATAC | (SEQ | GGCT | (SEQ | GACC | LSF |
| | TTTGGGTAACACACTAAAGA | EEAFLTINCTYT | TGACTCTCTCA | WYVQYPGEGL | CCTTC | ID NO: | GATG | ID NO: | GACT | (SEQ |
| | TGAACTATTCTCCAGGCTTA | ATGYPSLFWYV | GAAGAGGCCT | QLLLKATKADD | C | 206) | ACAA | 208) | ACAA | ID NO: |
| | GTATCTCTGATACTCTTACTG | QYPGEGLQLLL | TCCTGACTATA | KGSNKGFEATY | (SEQ | | G | | GCTC | 210) |
| | CTTGGAAGAACCCGTGGAAA | KATKADDKGS | AACTGCACGTA | RKETTSFHLEK | ID NO: | | (SEQ | | AGC | |
| | TTCAGTGACCCAGATGGAAG | NKGFEATYRKE | CACAGCCACA | GSVQVSDSAVY | 205) | | ID NO: | | (SEQ | |
| | GGCCAGTGACTCTCTCAGAA | TTSFHLEKGSV | GGATACCCTTC | FCALMTDYKLS | | | 207) | | ID NO: | |
| | GAGGCCTTCCTGACTATAAA | QVSDSAVYFCA | CCTTTTCTGGT | FGAGTTVTVRA | | | | | 209) | |
| | CTGCACGTACACAGCCACAG | LMTDYKLSFGA | ATGTCCAATAT | (SEQ ID NO: | | | | | | |
| | GATACCCTTCCCTTTTCTGGT | GTTVTVRANIQ | CCTGGAGAAG | 566) | | | | | | |
| | ATGTCCAATATCCTGGAGAA | NPDPAVYQLR | GTCTACAGCTC | | | | | | | |
| | GGTCTACAGCTCCTCCTGAA | DSKSSDKSVCL | CTCCTGAAAGC | | | | | | | |
| | AGCCACGAAGGCTGATGACA | FTDFDSQTNVS | CACGAAGGCT | | | | | | | |
| | AGGGAAGCAACAAAGGTTTT | QSKDSDVYITD | GATGACAAGG | | | | | | | |
| | GAAGCCACATACCGTAAAGA | KTVLDMRSMD | GAAGCAACAA | | | | | | | |
| | AACCACTTCTTTCCACTTGGA | FKSNSAVAWS | AGGTTTTGAAG | | | | | | | |
| | GAAAGGCTCAGTTCAAGTGT | NKSDFACANA | CCACATACCGT | | | | | | | |
| | CAGACTCAGCGGTGTACTTC | FNNSIIPEDTFF | AAAGAAACCA | | | | | | | |
| | TGTGCTCTGATGACCGACTA | PSPESSCDVKL | CTTCTTTCCAC | | | | | | | |
| | CAAGCTCAGCTTTGGAGCCG | VEKSFETDTNL | TTGGAGAAAG | | | | | | | |
| | GAACCACAGTAACTGTAAGA | NFQNLSVIGFR | GCTCAGTTCAA | | | | | | | |
| | GCAAATATCCAGAACCCTG | ILLLKVAGFNL | GTGTCAGACTC | | | | | | | |
| | ACCCTGCCGTGTACCAGCT | LMTLRLWSS | AGCGGTGTACT | | | | | | | |
| | GAGAGACTCTAAATCCAGT | (SEQ ID NO: | TCTGTGCTCTG | | | | | | | |
| | GACAAGTCTGTCTGCCTAT | 564) | ATGACCGACTA | | | | | | | |
| | TCACCGATTTTGATTCTCA | | CAAGCTCAGCT | | | | | | | |
| | AACAAATGTGTCACAAAGT | | TTGGAGCCGG | | | | | | | |
| | AAGGATTCTGATGTGTATA | | AACCACAGTA | | | | | | | |
| | TCACAGACAAAACTGTGCT | | ACTGTAAGAG | | | | | | | |
| | AGACATGAGGTCTATGGAC | | CAA | | | | | | | |
| | TTCAAGAGCAACAGTGCTG | | (SEQ ID NO: | | | | | | | |
| | TGGCCTGGAGCAACAAATC | | 565) | | | | | | | |
| | TGACTTTGCATGTGCAAAC | | | | | | | | | |
| | GCCTTCAACAACAGCATTA | | | | | | | | | |
| | TTCCAGAAGACACCTTCTT | | | | | | | | | |
| | CCCCAGCCCAGAAAGTTCC | | | | | | | | | |
| | TGTGATGTCAAGCTGGTCG | | | | | | | | | |
| | AGAAAAGCTTTGAAACAGA | | | | | | | | | |
| | TACGAACCTAAACTTTCAA | | | | | | | | | |
| | AACCTGTCAGTGATTGGGT | | | | | | | | | |
| | TCCGAATCCTCCTCCTGAA | | | | | | | | | |
| | AGTGGCCGGGTTTAATCTG | | | | | | | | | |
| | CTCATGACGCTGCGGCTGT | | | | | | | | | |
| | GGTCCAGC | | | | | | | | | |
| | (SEQ ID NO: 204) | | | | | | | | | |
| 31 | GGGAGTGGTGAATTAGGGGT | MMKSLRVLLVI | CAGAAGGAGG | QKEVEQNSGPL | GACC | DRGS | ATAT | IYSNG | GCCG | CAVR |
| | GTTAAAAAGAGCATCATTTT | LWLQLSWVWS | TGGAGCAGAA | SVPEGAIASLNC | GAGG | QS | ACTC | D | TGAG | SVGV |
| | TTTGAACTGGTAAAGCAGAT | QQKEVEQNSGP | TTCTGGACCCC | TYSDRGSQSFF | TTCCC | (SEQ | CAAT | (SEQ | GAGT | SGGY |
| | TCTTTTTATGATTTTTAAAGT | LSVPEGAIASLN | TCAGTGTTCCA | WYRQYSGKSPE | AGTC | ID NO: | GGTG | ID NO: | GTAG | NKLIF |
| | AGAAATATCCATTCCAGGTG | CTYSDRGSQSF | GAGGGAGCCA | LIMSIYSNGDKE | C | 213) | AC | 215) | GGGT | (SEQ |
| | CATTTTTTAAGGGTTTAAAAT | FWYRQYSGKSP | TTGCCTCTCTC | DGRFTAQLNKA | (SEQ | | (SEQ | | TTCTG | ID NO: |
| | TTGAATCCTCAGTGAACCAG | ELIMSIYSNGDK | AACTGCACTTA | SQYVSLLIRDSQ | ID NO: | | ID NO: | | GTGG | 217) |
| | GGCAGAGAAGAATGATGAA | EDGRFTAQLNK | CAGTGACCGA | PSDSATYLCAV | 212) | | 214) | | CTAC | |
| | ATCCTTGAGAGTTTTACTAGT | ASQYVSLLIRDS | GGTTCCCAGTC | RSVGVSGGYNK | | | | | AATA | |
| | GATCCTGTGGCTTCAGTTGA | QPSDSATYLCA | CTTCTTCTGGT | LIFGAGTRLAV | | | | | AGCT | |
| | GCTGGGTTTGGAGCCAACAG | VRSVGVSGGYN | ACAGACAATA | HP | | | | | GATT | |
| | AAGGAGGTGGAGCAGAATTC | KLIFGAGTRLA | TTCTGGGAAAA | (SEQ ID NO: | | | | | (SEQ | |
| | TGGACCCCTCAGTGTTCCAG | VHPYIQNPDPA | GCCCTGAGTTG | 569) | | | | | ID NO: | |
| | AGGGAGCCATTGCCTCTCTC | VYQLRDSKSSD | ATAATGTCCAT | | | | | | 216) | |
| | AACTGCACTTACAGTGACCG | KSVCLFTDFDS | ATACTCCAATG | | | | | | | |
| | AGGTTCCCAGTCCTTCTTCTG | QTNVSQSKDSD | GTGACAAAGA | | | | | | | |
| | GTACAGACAATATTCTGGGA | VYITDKTVLD | AGATGGAAGG | | | | | | | |
| | AAAGCCCTGAGTTGATAATG | MRSMDFKSNS | TTTACAGCACA | | | | | | | |
| | TCCATATACTCCAATGGTGA | AVAWSNKSDF | GCTCAATAAA | | | | | | | |
| | CAAAGAAGATGGAAGGTTTA | ACANAFNNSII | GCCAGCCAGT | | | | | | | |
| | CAGCACAGCTCAATAAAGCC | PEDTFFPSPESS | ATGTTTCTCTG | | | | | | | |
| | AGCCAGTATGTTTCTCTGCTC | CDVKLVEKSF | CTCATCAGAGA | | | | | | | |
| | ATCAGAGACTCCCAGCCCAG | ETDTNLNFQN | CTCCCAGCCCA | | | | | | | |
| | TGATTCAGCCACCTACCTCT | LSVIGFRILLL | GTGATTCAGCC | | | | | | | |
| | GTGCCGTGAGGAGTGTAGGG | KVAGFNLLMT | ACCTACCTCTG | | | | | | | |
| | GTTTCTGGTGGCTACAATAA | LRLWSS | TGCCGTGAGG | | | | | | | |
| | GCTGATTTTTGGAGCAGGGA | (SEQ ID NO: | AGTGTAGGGG | | | | | | | |
| | CCAGGCTGGCTGTACACCCA | 567) | TTTCTGGTGGC | | | | | | | |
| | TATATCCAGAACCCTGACC | | TACAATAAGCT | | | | | | | |
| | CTGCCGTGTACCAGCTGAG | | GATTTTTGGAG | | | | | | | |
| | AGACTCTAAATCCAGTGAC | | CAGGGACCAG | | | | | | | |
| | AAGTCTGTCTGCCTATTCA | | GCTGGCTGTAC | | | | | | | |
| | CCGATTTTGATTCTCAAAC | | ACCCAT | | | | | | | |
| | AAATGTGTCACAAAGTAAG | | (SEQ ID NO: | | | | | | | |
| | GATTCTGATGTGTATATCA | | 568) | | | | | | | |
| | CAGACAAAACTGTGCTAGA | | | | | | | | | |
| | CATGAGGTCTATGGACTTC | | | | | | | | | |
| | AAGAGCAACAGTGCTGTGG | | | | | | | | | |
| | CCTGGAGCAACAAATCTGA | | | | | | | | | |
| | CTTTGCATGTGCAAACGCC | | | | | | | | | |
| | TTCAACAACAGCATTATTC | | | | | | | | | |
| | CAGAAGACACCTTCTTCCC | | | | | | | | | |
| | CAGCCCAGAAAGTTCCTGT | | | | | | | | | |
| | GATGTCAAGCTGGTCGAGA | | | | | | | | | |
| | AAAGCTTTGAAACAGATAC | | | | | | | | | |
| | GAACCTAAACTTTCAAAAC | | | | | | | | | |
| | CTGTCAGTGATTGGGTTCC | | | | | | | | | |
| | GAATCCTCCTCCTGAAAGT | | | | | | | | | |
| | GGCCGGGTTTAATCTGCTC | | | | | | | | | |
| | ATGACGCTGCGGCTGTGGT | | | | | | | | | |
| | CCAGC | | | | | | | | | |
| | (SEQ ID NO: 211) | | | | | | | | | |
| 32 | GGGTGACAGCTGCTGGTGTG | MRQVARVIVFL | CTTGCTAAGAC | LAKTTQPISMD | AACA | NIATN | GGAT | GYKT | CTCG | CLVLL |
| | GGCCCTGGCAGTTGCTGCTG | TLSTLSLAKTTQ | CACCCAGCCCA | SYEGQEVNITCS | TTGCT | DY | ACAA | K | TTCTA | SSNTG |
| | GGCTCATTGCAGCTCAGACA | PISMDSYEGQE | TCTCCATGGAC | HNNIATNDYIT | ACAA | (SEQ | GACA | (SEQ | CTCTC | KLIF |
| | CAGCAAAAGAGCCTAGAACC | VNITCSHNNIAT | TCATATGAAGG | WYQQFPSQGPR | ATGA | ID NO: | AAA | ID NO: | TAGC | (SEQ |
| | TGGGTCCTAGTTTGCACCTA | NDYITWYQQFP | ACAAGAAGTG | FIIQGYKTKVTN | TTAT | 220) | (SEQ | 222) | AACA | ID NO: |
| | GAATATGAGGCAAGTGGCGA | SQGPRFIIQGYK | AACATAACCTG | EVASLFIPADRK | (SEQ | | ID NO: | | CAGG | 224) |
| | GAGTGATCGTGTTCCTGACC | TKVTNEVASLFI | TAGCCACAAC | SSTLSLPRVSLS | ID NO: | | 221) | | CAAA | |
| | CTGAGTACTTTGAGCCTTGCT | PADRKSSTLSLP | AACATTGCTAC | DTAVYYCLVLL | 219) | | | | CTAA | |
| | AAGACCACCCAGCCCATCTC | RVSLSDTAVYY | AAATGATTATA | SSNTGKLIFGQG | | | | | TC | |
| | CATGGACTCATATGAAGGAC | CLVLLSSNTGK | TCACGTGGTAC | TTLQVKP | | | | | (SEQ | |
| | AAGAAGTGAACATAACCTGT | LIFGQGTTLQV | CAACAGTTTCC | (SEQ ID NO: | | | | | ID NO: | |
| | AGCCACAACAACATTGCTAC | KPDIQNPDPAV | CAGCCAAGGA | 572) | | | | | 223) | |
| | AAATGATTATATCACGTGGT | YQLRDSKSSDK | CCACGATTTAT | | | | | | | |
| | ACCAACAGTTTCCCAGCCAA | SVCLFTDFDSQ | TATTCAAGGAT | | | | | | | |
| | GGACCACGATTTATTATTCA | TNVSQSKDSDV | ACAAGACAAA | | | | | | | |
| | AGGATACAAGACAAAAGTTA | YITDKTVLDM | AGTTACAAAC | | | | | | | |
| | CAAACGAAGTGGCCTCCCTG | RSMDFKSNSA | GAAGTGGCCTC | | | | | | | |
| | TTTATCCCTGCCGACAGAAA | VAWSNKSDFA | CCTGTTTATCC | | | | | | | |
| | GTCCAGCACTCTGAGCCTGC | CANAFNNSIIPE | CTGCCGACAG | | | | | | | |
| | CCCGGGTTTCCCTGAGCGAC | DTFFPSPESSC | AAAGTCCAGC | | | | | | | |
| | ACTGCTGTGTACTACTGCCTC | DVKLVEKSFET | ACTCTGAGCCT | | | | | | | |
| | GTTCTACTCTCTAGCAACAC | DTNLNFQNLSV | GCCCCGGGTTT | | | | | | | |
| | AGGCAAACTAATCTTTGGGC | IGFRILLLKVA | CCCTGAGCGAC | | | | | | | |
| | AAGGGACAACTTTACAAGTA | GFNLLMTLRL | ACTGCTGTGTA | | | | | | | |
| | AAACCAGATATCCAGAACC | WSS | CTACTGCCTCG | | | | | | | |
| | CTGACCCTGCCGTGTACCA | (SEQ ID NO: | TTCTACTCTCT | | | | | | | |
| | GCTGAGAGACTCTAAATCC | 570) | AGCAACACAG | | | | | | | |
| | AGTGACAAGTCTGTCTGCC | | GCAAACTAATC | | | | | | | |
| | TATTCACCGATTTTGATTCT | | TTTGGGCAAGG | | | | | | | |
| | CAAACAAATGTGTCACAAA | | GACAACTTTAC | | | | | | | |
| | GTAAGGATTCTGATGTGTA | | AAGTAAAACC | | | | | | | |
| | TATCACAGACAAAACTGTG | | AG | | | | | | | |
| | CTAGACATGAGGTCTATGG | | (SEQ ID NO: | | | | | | | |
| | ACTTCAAGAGCAACAGTGC | | 571) | | | | | | | |
| | TGTGGCCTGGAGCAACAAA | | | | | | | | | |
| | TCTGACTTTGCATGTGCAA | | | | | | | | | |
| | ACGCCTTCAACAACAGCAT | | | | | | | | | |
| | TATTCCAGAAGACACCTTC | | | | | | | | | |
| | TTCCCCAGCCCAGAAAGTT | | | | | | | | | |
| | CCTGTGATGTCAAGCTGGT | | | | | | | | | |
| | CGAGAAAAGCTTTGAAACA | | | | | | | | | |
| | GATACGAACCTAAACTTTC | | | | | | | | | |
| | AAAACCTGTCAGTGATTGG | | | | | | | | | |
| | GTTCCGAATCCTCCTCCTG | | | | | | | | | |
| | AAAGTGGCCGGGTTTAATC | | | | | | | | | |
| | TGCTCATGACGCTGCGGCT | | | | | | | | | |
| | GTGGTCCAGC | | | | | | | | | |
| | (SEQ ID NO: 218) | | | | | | | | | |
| 33 | TGGGGCTGTTCTGAAGCAGC | MACPGFLWAL | GCTCAGACAGT | AQTVTQSQPEM | ACCA | TSESD | CAAG | QEAY | GCTT | CAYR |
| | TACGGCACCAGTGCAGCTGA | VISTCLEFSMAQ | CACTCAGTCTC | SVQEAETVTLS | GTGA | YY | AAGC | KQQN | ATAG | SANN |
| | TACTCAAGGTTCAGATCAGA | TVTQSQPEMSV | AACCAGAGAT | CTYDTSESDYY | GAGT | (SEQ | TTAT | (SEQ | GAGC | NDMR |
| | AGAGGAGGCTTCTCACCCTG | QEAETVTLSCT | GTCTGTGCAGG | LFWYKQPPSRQ | GATT | ID NO: | AAGC | ID NQ | GCGA | F |
| | CAGCAGGGACCTGTGAGCAT | YDTSESDYYLF | AGGCAGAGAC | MILVIRQEAYK | ATTA | 227) | AACA | 229) | ATAA | (SEQ |
| | GGCATGCCCTGGCTTCCTGT | WYKQPPSRQMI | CGTGACCCTGA | QQNATENRFSV | T | | GAAT | | CAAT | ID NO: |
| | GGGCACTTGTGATCTCCACC | LVIRQEAYKQQ | GCTGCACATAT | NFQKAAKSFSL | (SEQ | | (SEQ | | GACA | 231) |
| | TGTCTTGAATTTAGCATGGCT | NATENRFSVNF | GACACCAGTG | KISDSQLGDAA | ID NO: | | ID NO: | | TGCG | |
| | CAGACAGTCACTCAGTCTCA | QKAAKSFSLKIS | AGAGTGATTAT | MYFCAYRSAN | 226) | | 228) | | C | |
| | ACCAGAGATGTCTGTGCAGG | DSQLGDAAMY | TATTTATTCTG | NNDMRFGAGT | | | | | (SEQ | |
| | AGGCAGAGACCGTGACCCTG | FCAYRSANNND | GTACAAGCAG | RLTVKP | | | | | ID NO: | |
| | AGCTGCACATATGACACCAG | MRFGAGTRLTV | CCTCCCAGCAG | (SEQ ID NO: | | | | | 230) | |
| | TGAGAGTGATTATTATTTATT | KPNIQNPDPAV | GCAGATGATTC | 575) | | | | | | |
| | CTGGTACAAGCAGCCTCCCA | YQLRDSKSSDK | TCGTTATTCGC | | | | | | | |
| | GCAGGCAGATGATTCTCGTT | SVCLFTDFDSQ | CAAGAAGCTT | | | | | | | |
| | ATTCGCCAAGAAGCTTATAA | TNVSQSKDSDV | ATAAGCAACA | | | | | | | |
| | GCAACAGAATGCAACAGAG | YITDKTVLDM | GAATGCAACA | | | | | | | |
| | AATCGTTTCTCTGTGAACTTC | RSMDFKSNSA | GAGAATCGTTT | | | | | | | |
| | CAGAAAGCAGCCAAATCCTT | VAWSNKSDFA | CTCTGTGAACT | | | | | | | |
| | CAGTCTCAAGATCTCAGACT | CANAFNNSIIPE | TCCAGAAAGC | | | | | | | |
| | CACAGCTGGGGGATGCCGCG | DTFFPSPESSC | AGCCAAATCCT | | | | | | | |
| | ATGTATTTCTGTGCTTATAGG | DVKLVEKSFET | TCAGTCTCAAG | | | | | | | |
| | AGCGCGAATAACAATGACAT | DTNLNFQNLSV | ATCTCAGACTC | | | | | | | |
| | GCGCTTTGGAGCAGGGACCA | IGFRILLLKVA | ACAGCTGGGG | | | | | | | |
| | GACTGACAGTAAAACCAAAT | GFNLLMTLRL | GATGCCGCGAT | | | | | | | |
| | ATCCAGAACCCTGACCCTG | WSS | GTATTTCTGTG | | | | | | | |
| | CCGTGTACCAGCTGAGAGA | (SEQ ID NO: | CTTATAGGAGC | | | | | | | |
| | CTCTAAATCCAGTGACAAG | 573) | GCGAATAACA | | | | | | | |
| | TCTGTCTGCCTATTCACCG | | ATGACATGCGC | | | | | | | |
| | ATTTTGATTCTCAAACAAA | | TTTGGAGCAGG | | | | | | | |
| | TGTGTCACAAAGTAAGGAT | | GACCAGACTG | | | | | | | |
| | TCTGATGTGTATATCACAG | | ACAGTAAAAC | | | | | | | |
| | ACAAAACTGTGCTAGACAT | | CAA | | | | | | | |
| | GAGGTCTATGGACTTCAAG | | (SEQ ID NO: | | | | | | | |
| | AGCAACAGTGCTGTGGCCT | | 574) | | | | | | | |
| | GGAGCAACAAATCTGACTT | | | | | | | | | |
| | TGCATGTGCAAACGCCTTC | | | | | | | | | |
| | AACAACAGCATTATTCCAG | | | | | | | | | |
| | AAGACACCTTCTTCCCCAG | | | | | | | | | |
| | CCCAGAAAGTTCCTGTGAT | | | | | | | | | |
| | GTCAAGCTGGTCGAGAAAA | | | | | | | | | |
| | GCTTTGAAACAGATACGAA | | | | | | | | | |
| | CCTAAACTTTCAAAACCTG | | | | | | | | | |
| | TCAGTGATTGGGTTCCGAA | | | | | | | | | |
| | TCCTCCTCCTGAAAGTGGC | | | | | | | | | |
| | CGGGTTTAATCTGCTCATG | | | | | | | | | |
| | ACGCTGCGGCTGTGGTCCA | | | | | | | | | |
| | GC | | | | | | | | | |
| | (SEQ ID NO: 225) | | | | | | | | | |
| 34 | GGGGGTGTGTGTGTGTGCTT | MMISLRVLLVIL | CGGAAGGAGG | RKEVEQDPGPF | AACA | NSAS | GTAT | VYSS | GTGG | CVVFP |
| | GAGAGAGAGAGAAGGAGAG | WLQLSWVWSQ | TGGAGCAGGA | NVPEGATVAFN | GTGC | QS | ACTC | GN | TTTTC | GGSYI |
| | AAAGAGAGAGAATGGGAAG | RKEVEQDPGPF | TCCTGGACCCT | CTYSNSASQSFF | TTCTC | (SEQ | CAGT | (SEQ | CCAG | PTF |
| | GGCGATAAGAGGAGGGGTT | NVPEGATVAFN | TCAATGTTCCA | WYRQDCRKEP | AGTC | ID NO: | GGTA | ID NO: | GAGG | (SEQ |
| | AGTGATATACCAGGGGTTGT | CTYSNSASQSFF | GAGGGAGCCA | KLLMSVYSSGN | T | 234) | AT | 236) | AAGC | ID NO: |
| | GAAAATAACCTCTTTTTTCTA | WYRQDCRKEP | CTGTCGCTTTC | EDGRFTAQLNR | (SEQ | | (SEQ | | TACA | 238) |
| | ATTGGTAGGACAGATTCTTT | KLLMSVYSSGN | AACTGTACTTA | ASQYISLLIRDS | ID NO: | | ID NO: | | TACC | |
| | TTATGATTCCTAAAGTGGAA | EDGRFTAQLNR | CAGCAACAGT | KLSDSATYLCV | 233) | | 235) | | TACA | |
| | GAAATAAAGTATCTCTGCTA | ASQYISLLIRDS | GCTTCTCAGTC | VFPGGSYIPTFG | | | | | (SEQ | |
| | TGTTCATTTCTTTTTGGATTG | KLSDSATYLCV | TTTCTTCTGGT | RGTSLIVHP | | | | | ID NO: | |
| | AAAATTTTAATCCTCAGTGA | VFPGGSYIPTFG | ACAGACAGGA | (SEQ ID NO: | | | | | 237) | |
| | ACCAGGGCAGAAAAGAATG | RGTSLIVHPYIQ | TTGCAGGAAA | 578) | | | | | | |
| | ATGATATCCTTGAGAGTTTT | KPDPAVYQLR | GAACCTAAGTT | | | | | | | |
| | ACTGGTGATCCTGTGGCTTC | DSKSSDKSVCL | GCTGATGTCCG | | | | | | | |
| | AGTTAAGCTGGGTTTGGAGC | FTDFDSQTNVS | TATACTCCAGT | | | | | | | |
| | CAACGGAAGGAGGTGGAGC | QSKDSDVYITD | GGTAATGAAG | | | | | | | |
| | AGGATCCTGGACCCTTCAAT | KTVLDMRSMD | ATGGAAGGTTT | | | | | | | |
| | GTTCCAGAGGGAGCCACTGT | FKSNSAVAWS | ACAGCACAGC | | | | | | | |
| | CGCTTTCAACTGTACTTACA | NKSDFACANA | TCAATAGAGCC | | | | | | | |
| | GCAACAGTGCTTCTCAGTCT | FNNSIIPEDTFF | AGCCAGTATAT | | | | | | | |
| | TTCTTCTGGTACAGACAGGA | PSPESSCDVKL | TTCCCTGCTCA | | | | | | | |
| | TTGCAGGAAAGAACCTAAGT | VEKSFETDTNL | TCAGAGACTCC | | | | | | | |
| | TGCTGATGTCCGTATACTCC | NFQNLSVIGFR | AAGCTCAGTG | | | | | | | |
| | AGTGGTAATGAAGATGGAAG | ILLLKVAGFNL | ATTCAGCCACC | | | | | | | |
| | GTTTACAGCACAGCTCAATA | LMTLRLWSS | TACCTCTGTGT | | | | | | | |
| | GAGCCAGCCAGTATATTTCC | (SEQ ID NO: | GGTTTTCCCAG | | | | | | | |
| | CTGCTCATCAGAGACTCCAA | 576) | GAGGAAGCTA | | | | | | | |
| | GCTCAGTGATTCAGCCACCT | | CATACCTACAT | | | | | | | |
| | ACCTCTGTGTGGTTTTCCCAG | | TTGGAAGAGG | | | | | | | |
| | GAGGAAGCTACATACCTACA | | AACCAGCCTTA | | | | | | | |
| | TTTGGAAGAGGAACCAGCCT | | TTGTTCATCCG | | | | | | | |
| | TATTGTTCATCCGTATATCC | | T | | | | | | | |
| | AGAAGCCTGACCCTGCCGT | | (SEQ ID NO: | | | | | | | |
| | GTACCAGCTGAGAGACTCT | | 577) | | | | | | | |
| | AAATCCAGTGACAAGTCTG | | | | | | | | | |
| | TCTGCCTATTCACCGATTT | | | | | | | | | |
| | TGATTCTCAAACAAATGTG | | | | | | | | | |
| | TCACAAAGTAAGGATTCTG | | | | | | | | | |
| | ATGTGTATATCACAGACAA | | | | | | | | | |
| | AACTGTGCTAGACATGAGG | | | | | | | | | |
| | TCTATGGACTTCAAGAGCA | | | | | | | | | |
| | ACAGTGCTGTGGCCTGGAG | | | | | | | | | |
| | CAACAAATCTGACTTTGCA | | | | | | | | | |
| | TGTGCAAACGCCTTCAACA | | | | | | | | | |
| | ACAGCATTATTCCAGAAGA | | | | | | | | | |
| | CACCTTCTTCCCCAGCCCA | | | | | | | | | |
| | GAAAGTTCCTGTGATGTCA | | | | | | | | | |
| | AGCTGGTCGAGAAAAGCTT | | | | | | | | | |
| | TGAAACAGATACGAACCTA | | | | | | | | | |
| | AACTTTCAAAACCTGTCAG | | | | | | | | | |
| | TGATTGGGTTCCGAATCCT | | | | | | | | | |
| | CCTCCTGAAAGTGGCCGGG | | | | | | | | | |
| | TTTAATCTGCTCATGACGC | | | | | | | | | |
| | TGCGGCTGTGGTCCAGC | | | | | | | | | |
| | (SEQ ID NO: 232) | | | | | | | | | |

TABLE 5

| | | TCR BETA | | | |
|---|---|---|---|---|---|
| | TCR BETA (FULL LENGTH) | (FULL LENGTH) | TCR BETA (VDJ | TCR BETA (VDJ | CDR1 |
| ID | (NT) | (AA) | REGION) (NT) | REGION) (AA) | (NT) |
| 1 | AGAAAGCAGAATGGGTGAT | MATRLLCCVVL | GATGCTAGAGT | DARVTQTPRHK | TTAG |
| | GTGATGTGCAATGCCACAGA | CLLGEELIDAR | CACCCAGACA | VTEMGQEVTM | GCCA |
| | AGCACTGCAGCCAGGAGAG | VTQTPRHKVTE | CCAAGGCACA | RCQPILGHNTV | CAAT |
| | GTGACAGCTAATGGGGATGT | MGQEVTMRCQ | AGGTGACAGA | FWYRQTMMQG | ACT |
| | TTGGAGTCTTTGAGTGAACC | PILGHNTVFWY | GATGGGACAA | LELLAYFRNRA | (SEQ |
| | AAACACATCCCAGAGTAATT | RQTMMQGLEL | GAAGTAACAA | PLDDSGMPKDR | ID NO: |
| | GTAATTTATTTCAGTCAATCT | LAYFRNRAPLD | TGAGATGTCAG | FSAEMPDATLA | 240) |
| | TCTGTACAGACTTAGCATTC | DSGMPKDRFSA | CCAATTTTAGG | TLKIQPSEPRDS | |
| | ACCTTTGGAGGAAGGTCCTT | EMPDATLATLK | CCACAATACTG | AVYFCASGLAL | |
| | TGAGCAGGGACAGAGATGGT | IQPSEPRDSAVY | TTTTCTGGTAC | TEGGWYEQYF | |
| | GATGTCACTGACAGTCCCCC | FCASGLALTEG | AGACAGACCA | GPGTRLTVT | |
| | TTTTACTCTGGGTGAGAGGT | GWYEQYFGPG | TGATGCAAGG | (SEQ ID NO: | |
| | CTAGAATCCTCAGCTCCTGT | TRLTVTEDLKN | ACTGGAGTTGC | 581) | |
| | ATTCGTGCCCACAAGGGCCT | VFPPEVAVFEP | TGGCTTACTTC | | |
| | CATCTAGGTGAAGGCTCCAC | SEAEISHTQKA | CGCAACCGGG | | |
| | CTGCCCCACCCTGCCATGGC | TLVCLATGFYP | CTCCTCTAGAT | | |
| | CACCAGGCTCCTCTGCTGTG | DHVELSWWVN | GATTCGGGGAT | | |
| | TGGTTCTTTGTCTCCTGGGAG | GKEVHSGVST | GCCGAAGGAT | | |
| | AAGAGCTTATAGATGCTAGA | DPQPLKEQPA | CGATTCTCAGC | | |
| | GTCACCCAGACACCAAGGCA | LNDSRYCLSSR | AGAGATGCCT | | |
| | CAAGGTGACAGAGATGGGA | LRVSATFWQN | GATGCAACTTT | | |
| | CAAGAAGTAACAATGAGATG | PRNHERCQVQ | AGCCACTCTGA | | |
| | TCAGCCAATTTTAGGCCACA | FYGLSENDEW | AGATCCAGCCC | | |
| | ATACTGTTTTCTGGTACAGA | TQDRAKPVTQI | TCAGAACCCA | | |
| | CAGACCATGATGCAAGGACT | VSAEAWGRAD | GGGACTCAGCT | | |
| | GGAGTTGCTGGCTTACTTCC | CGFTSESYQQ | GTGTATTTTTG | | |
| | GCAACCGGGCTCCTCTAGAT | GVLSATILYEI | TGCTAGTGGTT | | |
| | GATTCGGGGATGCCGAAGGA | LLGKATLYAV | TGGCCCTCACT | | |
| | TCGATTCTCAGCAGAGATGC | LVSALVLMAM | GAGGGGGGCT | | |
| | CTGATGCAACTTTAGCCACT | VKRKDSRG | GGTACGAGCA | | |
| | CTGAAGATCCAGCCCTCAGA | (SEQ ID NO: | GTACTTCGGGC | | |
| | ACCCAGGGACTCAGCTGTGT | 579) | CGGGCACCAG | | |
| | ATTTTTGTGCTAGTGGTTTGG | | GCTCACGGTCA | | |
| | CCCTCACTGAGGGGGGCTGG | | CAG | | |
| | TACGAGCAGTACTTCGGGCC | | (SEQ ID NO: | | |
| | GGGCACCAGGCTCACGGTCA | | 580) | | |
| | CAGAGGACCTGAAAAACGT | | | | |
| | GTTCCCACCCGAGGTCGCT | | | | |
| | GTGTTTGAGCCATCAGAAG | | | | |
| | CAGAGATCTCCCACACCCA | | | | |
| | AAAGGCCACACTGGTATGC | | | | |
| | CTGGCCACAGGCTTCTACC | | | | |
| | CCGACCACGTGGAGCTGAG | | | | |
| | CTGGTGGGTGAATGGGAA | | | | |
| | GGAGGTGCACAGTGGGGT | | | | |
| | CAGCACAGACCCGCAGCCC | | | | |
| | CTCAAGGAGCAGCCCGCCC | | | | |
| | TCAATGACTCCAGATACTG | | | | |
| | CCTGAGCAGCCGCCTGAGG | | | | |
| | GTCTCGGCCACCTTCTGGC | | | | |
| | AGAACCCCCGCAACCACTT | | | | |
| | CCGCTGTCAAGTCCAGTTC | | | | |
| | TACGGGCTCTCGGAGAATG | | | | |
| | ACGAGTGGACCCAGGATAG | | | | |
| | GGCCAAACCTGTCACCCAG | | | | |
| | ATCGTCAGCGCCGAGGCCT | | | | |
| | GGGGTAGAGCAGACTGTG | | | | |
| | GCTTCACCTCCGAGTCTTA | | | | |
| | CCAGCAAGGGGTCCTGTCT | | | | |
| | GCCACCATCCTCTATGAGA | | | | |
| | TCTTGCTAGGGAAGGCCAC | | | | |
| | CTTGTATGCCGTGCTGGTC | | | | |
| | AGTGCCCTCGTGCTGATGG | | | | |
| | CCATGGTCAAGAGAAAGGA | | | | |
| | TTCCAGAGGCTAG (SEQ ID | | | | |
| | NO: 239) | | | | |
| 2 | GGGCCAGAGACACCAGTAAT | MDTWLVCWAI | GAACCTGAAG | EPEVTQTPSHQ | TCTA |
| | TCTGCCAGACCTTGCCTGTG | FSLLKAGLTEPE | TCACCCAGACT | VTQMGQEVILR | ATCA |
| | GGGCCATGGGAGCTCAAAAT | VTQTPSHQVTQ | CCCAGCCATCA | CVPISNHLYFY | CTTAT |
| | GCCCCTCCTTTCCTCCACAGG | MGQEVILRCVPI | GGTCACACAG | WYRQILGQKVE | AC |
| | ACCAGATGCCTGAGCTAGGA | SNHLYFYWYR | ATGGGACAGG | FLVSFYNNEISE | (SEQ |
| | AAGGCCTCATTCCTGCTGTG | QILGQKVEFLV | AAGTGATCTTG | KSEIFDDQFSVE | ID NO: |
| | ATCCTGCCATGGATACCTGG | SFYNNEISEKSE | CGCTGTGTCCC | RPDGSNFTLKIR | 247) |
| | CTCGTATGCTGGGCAATTTTT | IFDDQFSVERPD | CATCTCTAATC | STKLEDSAMYF | |
| | AGTCTCTTGAAAGCAGGACT | GSNFTLKIRSTK | ACTTATACTTC | CASLRWDGDN | |
| | CACAGAACCTGAAGTCACCC | LEDSAMYFCAS | TATTGGTACAG | EQFFGPGTRLT | |
| | AGACTCCCAGCCATCAGGTC | LRWDGDNEQFF | ACAAATCTTGG | VL | |
| | ACACAGATGGGACAGGAAG | GPGTRLTVLED | GGCAGAAAGT | (SEQ ID NO: | |
| | TGATCTTGCGCTGTGTCCCCA | LKNVFPPEVAV | CGAGTTTCTGG | 584) | |
| | TCTCTAATCACTTATACTTCT | FEPSEAEISHT | TTTCCTTTTAT | | |
| | ATTGGTACAGACAAATCTTG | QKATLVCLAT | AATAATGAAA | | |
| | GGGCAGAAAGTCGAGTTTCT | GFYPDHVELS | TCTCAGAGAA | | |
| | GGTTTCCTTTTATAATAATGA | WWVNGKEVH | GTCTGAAATAT | | |
| | AATCTCAGAGAAGTCTGAAA | SGVSTDPQPLK | TCGATGATCAA | | |
| | TATTCGATGATCAATTCTCA | EQPALNDSRY | TTCTCAGTTGA | | |
| | GTTGAAAGGCCTGATGGATC | CLSSRLRVSAT | AAGGCCTGAT | | |
| | AAATTTCACTCTGAAGATCC | FWQNPRNHFR | GGATCAAATTT | | |
| | GGTCCACAAAGCTGGAGGAC | CQVQFYGLSE | CACTCTGAAGA | | |
| | TCAGCCATGTACTTCTGTGCC | NDEWTQDRAK | TCCGGTCCACA | | |
| | AGCCTCCGATGGGACGGGGA | PVTQIVSAEAW | AAGCTGGAGG | | |
| | CAATGAGCAGTTCTTCGGGC | GRADCGFTSES | ACTCAGCCATG | | |
| | CAGGGACACGGCTCACCGTG | YQQGVLSATIL | TACTTCTGTGC | | |
| | CTAGAGGACCTGAAAAACG | YEILLGKATLY | CAGCCTCCGAT | | |
| | TGTTCCCACCCGAGGTCGC | AVLVSALVLM | GGGACGGGGA | | |
| | TGTGTTTGAGCCATCAGAA | AMVKRKDSRG | CAATGAGCAG | | |
| | GCAGAGATCTCCCACACCC | (SEQ ID NO: | TTCTTCGGGCC | | |
| | AAAAGGCCACACTGGTATG | 582) | AGGGACACGG | | |
| | CCTGCCACAGGCTTCTAC | | CTCACCGTGCT | | |
| | CCCGCCACGTGGAGCTGA | | AG | | |
| | GCTGGTGGGTGAATGGGA | | (SEQ ID NO: | | |
| | AGGAGGTGCACAGTGGGG | | 583) | | |
| | TCAGCACAGACCCGCAGCC | | | | |
| | CCTCAAGGAGCAGCCCGCC | | | | |
| | CTCAATGACTCCAGATACT | | | | |
| | GCCTGAGCAGCCGCCTGAG | | | | |
| | GGTCTCGGCCACCTTCTGG | | | | |
| | CAGAACCCCCGCAACCACT | | | | |
| | TCCGCTGTCAAGTCCAGTT | | | | |
| | CTACGGGCTCTCGGAGAAT | | | | |
| | GACGAGTGGACCCAGGATA | | | | |
| | GGGCCAAACCTGTCACCCA | | | | |
| | GATCGTCAGCGCCGAGGCC | | | | |
| | TGGGGTAGAGCAGACTGTG | | | | |
| | GCTTCACCTCCGAGTCTTA | | | | |
| | CCAGCAAGGGGTCCTGTCT | | | | |
| | GCCACCATCCTCTATGAGA | | | | |
| | TCTTGCTAGGGAAGGCCAC | | | | |
| | CTTGTATGCCGTGCTGGTC | | | | |
| | AGTGCCCTCGTGCTGATGG | | | | |
| | CCATGGTCAAGAGAAAGGA | | | | |
| | TTCCAGAGGCTAG (SEQ ID | | | | |
| | NO: 246) | | | | |
| 3 | GAATTTGACCATCTGGGGAA | MKSQNDPLEST | AATGCTGGTGT | NAGVTQTPKFQ | ATGA |
| | GGGGCGTGGCCTCTCCTGAC | VPLSPMHRPRR | CACTCAGACCC | VLKTGQSMTLQ | ACCA |
| | AGGAAGGCTCTGGGGCCCAG | PLHPVAPAMSI | CAAAATTCCAG | CAQDMNHNSM | TAAC |
| | GCAGGGAGAATGAAGTCTCA | GLLCCVAFSLL | GTCCTGAAGAC | YWYRQDPGMG | TCC |
| | GAATGACCCCCTTGAGAGTA | WASPVNAGVT | AGGACAGAGC | LRLIYYSASEGT | (SEQ |
| | CTGTTCCCCTATCACCGATGC | QTPKFQVLKTG | ATGACACTGCA | TDKGEVPNGYN | ID NO: |
| | ACAGACCCAGAAGACCCCTC | QSMTLQCAQD | GTGTGCCCAGG | VSRLNKREFSL | 254) |
| | CATCCTGTAGCACCTGCCAT | MNHNSMYWYR | ATATGAACCAT | RLESAAPSQTS | |
| | GAGCATCGGGCTCCTGTGCT | QDPGMGLRLIY | AACTCCATGTA | VYFCASRDRAL | |
| | GTGTGGCCTTTTCTCTCCTGT | YSASEGTTDKG | CTGGTATCGAC | NTEAFFGQGTR | |
| | GGGCAAGTCCAGTGAATGCT | EVPNGYNVSRL | AAGACCCAGG | LTVV | |
| | GGTGTCACTCAGACCCCAAA | NKREFSLRLES | CATGGGACTG | (SEQ ID NO: | |
| | ATTCCAGGTCCTGAAGACAG | AAPSQTSVYFC | AGGCTGATTTA | 587) | |
| | GACAGAGCATGACACTGCAG | ASRDRALNTEA | TTACTCAGCTT | | |
| | TGTGCCCAGGATATGAACCA | FFGQGTRLTVV | CTGAGGGTACC | | |
| | TAACTCCATGTACTGGTATC | EDLNKVFPPEV | ACTGACAAAG | | |
| | GACAAGACCCAGGCATGGG | AVFEPSEAEIS | GAGAAGTCCC | | |
| | ACTGAGGCTGATTTATTACT | HTQKATLVCL | CAATGGCTACA | | |
| | CAGCTTCTGAGGGTACCACT | ATGFYPDHVE | ATGTCTCCAGA | | |
| | GACAAAGGAGAAGTCCCCA | LSWWVNGKE | TTAAACAAAC | | |
| | ATGGCTACAATGTCTCCAGA | VHSGVSTDPQP | GGGAGTTCTCG | | |
| | TTAAACAAACGGGAGTTCTC | LKEQPALNDS | CTCAGGCTGGA | | |
| | GCTCAGGCTGGAGTCGGCTG | RYCLSSRLRVS | GTCGGCTGCTC | | |
| | CTCCCTCCCAGACATCTGTGT | ATFWQNPRNH | CCTCCCAGACA | | |
| | ACTTCTGTGCCAGCAGGGAC | FRCQVQFYGL | TCTGTGTACTT | | |
| | AGGGCCCTGAACACTGAAGC | SENDEWTQDR | CTGTGCCAGCA | | |
| | TTTCTTTGGACAAGGCACCA | AKPVTQIV | GGGACAGGGC | | |
| | GACTCACAGTTGTAGAGGAC | SAEAWGRADC | CCTGAACACTG | | |
| | CTGAACAAGGTGTTCCCAC | GFTSVSYQQG | AAGCTTTCTTT | | |
| | CCGAGGTCGCTGTGTTTGA | VLSATILYEILL | GGACAAGGCA | | |
| | GCCATCAGAAGCAGAGATC | GKATLYAVLV | CCAGACTCACA | | |
| | TCCCACACCCAAAAGGCCA | SALVLMAMVK | GTTGTAG | | |
| | CACTGGTGTGCCTGGCCAC | RKDF | (SEQ ID NO: | | |
| | AGGCTTCTACCCCGACCAC | (SEQ ID NO: | 586) | | |
| | GTGGAGCTGAGCTGGTGG | 585) | | | |
| | GTGAATGGGAAGGAGGTG | | | | |
| | CACAGTGGGGTCAGCACAG | | | | |
| | ACCCGCAGCCCCTCAAGGA | | | | |
| | GCAGCCCGCCCTCAATGAC | | | | |
| | TCCAGATACTGCCTGAGCA | | | | |
| | GCCGCCTGAGGGTCTCGGC | | | | |
| | CACCTTCTGGCAGAACCCC | | | | |
| | CGCAACCACTTCCGCTGTC | | | | |
| | AAGTCCAGTTCTACGGGCT | | | | |
| | CTCGGAGAATGACGAGTGG | | | | |
| | ACCCAGGATAGGGCCAAAC | | | | |
| | CCGTCCCCAGATCGTCAG | | | | |
| | CGCCGAGGCCTGGGGTAG | | | | |
| | AGCAGACTGTGGCTTTACC | | | | |
| | TCGGTGTCCTACCAGCAAG | | | | |
| | GGGTCCTGTCTGCCACCAT | | | | |
| | CCTCTATGAGATCCTGCTA | | | | |
| | GGGAAGGCCACCCTGTATG | | | | |
| | CTGTGCTGGTCAGCGCCCT | | | | |
| | TGTGTTGATGGCCATGGTC | | | | |
| | AAGAGAAAGGATTTCTGA | | | | |
| | (SEQ ID NO: 253) | | | | |
| 4 | GAAGAAGTCTCATCTGTCAG | MGVASPGLSLP | AATGCTGGTGT | NAGVTQTPKFQ | ATGA |
| | TGAGTTGACAAGAAACAGAG | GAQAGRMSQN | CACTCAGACCC | VLKTGQSMTLQ | ACCA |
| | CAAAACGACTCCTCCAATGT | DFLESPAPLSSM | CAAAATTCCAG | CAQDMNHEYM | TGAA |
| | TGACGAGCCTGCCCCTGGGA | HRYRRPLRHAA | GTCCTGAAGAC | SWYRQDPGMG | TAC |
| | TTTGGAAACTTCATAACAGA | SAMSIGLLCCA | AGGACAGAGC | LRLIHYSVGAGI | (SEQ |
| | AAAAACCAATATAGACAAA | ALSLLWAGPVN | ATGACACTGCA | TDQGEVPNGYN | ID NO: |
| | GGATTTTAAACAGGATTAAG | AGVTQTPKFQV | GTGTGCCCAGG | VSRSTTEDFPLR | 261) |
| | CTGTTCACTGGTGCATTTATT | LKTGQSMTLQC | ATATGAACCAT | LLSAAPSQTSV | |
| | TTGGATTTGACCATCTGGGG | AQDMNHEYMS | GAATACATGTC | YFCASRNGGTL | |
| | AATGGGTGTGGCCTCTCCTG | WYRQDPGMGL | CTGGTATCGAC | IYEQYFGPGTRL | |
| | GCCTCTCCCTCCCTGGGGCC | RLIHYSVGAGIT | AAGACCCAGG | TVT | |
| | CAGGCAGGGAGAATGTCTCA | DQGEVPNGYN | CATGGGGCTG | (SEQ ID NO: | |
| | GAATGACTTCCTTGAGAGTC | VSRSTTEDFPLR | AGGCTGATTCA | 590) | |
| | CTGCTCCCCTTTCATCAATGC | LLSAAPSQTSV | TTACTCAGTTG | | |
| | ACAGATACAGAAGACCCCTC | YFCASRNGGTL | GTGCTGGTATC | | |
| | CGTCATGCAGCATCTGCCAT | IYEQYFGPGTRL | ACTGACCAAG | | |
| | GAGCATCGGCCTCCTGTGCT | TVTEDLKNVFP | GAGAAGTCCC | | |
| | GTGCAGCCTTGTCTCTCCTGT | PEVAVFEPSEA | CAATGGCTACA | | |
| | GGGCAGGTCCAGTGAATGCT | EISHTQKATLV | ATGTCTCCAGA | | |
| | GGTGTCACTCAGACCCCAAA | CLATGFYPDH | TCAACCACAG | | |
| | ATTCCAGGTCCTGAAGACAG | VELSWWVNG | AGGATTTCCCG | | |
| | GACAGAGCATGACACTGCAG | KEVHSGVSTDP | CTCAGGCTGCT | | |
| | TGTGCCCAGGATATGAACCA | QPLKEQPALN | GTCGGCTGCTC | | |
| | TGAATACATGTCCTGGTATC | DSRYCLSSRLR | CCTCCCAGACA | | |
| | GACAAGACCCAGGCATGGG | VSATFWQNPR | TCTGTGTACTT | | |
| | GCTGAGGCTGATTCATTACT | NHFRCQVQFY | CTGTGCCAGCA | | |
| | CAGTTGGTGCTGGTATCACT | GLSENDEWTQ | GAAACGGCGG | | |
| | GACCAAGGAGAAGTCCCCAA | DRAKPVTQIVS | GACACTAATCT | | |
| | TGGCTACAATGTCTCCAGAT | AEAWGRADCG | ACGAGCAGTA | | |
| | CAACCACAGAGGATTTCCCG | FTSESYQQGVL | CTTCGGGCCGG | | |
| | CTCAGGCTGCTGTCGGCTGC | SATILYEILLG | GCACCAGGCTC | | |
| | TCCCTCCCAGACATCTGTGT | KATLYAVLVS | ACGGTCACAG | | |
| | ACTTCTGTGCCAGCAGAAAC | ALVLMAMVK | (SEQ ID NO: | | |
| | GGCGGGACACTAATCTACGA | RKDSRG | 589) | | |
| | GCAGTACTTCGGGCCGGGCA | (SEQ ID NO: | | | |
| | CCAGGCTCACGGTCACAGAG | 588) | | | |
| | GACCTGAAAAACGTGTTCC | | | | |
| | CACCCGAGGTCGCTGTGTT | | | | |
| | TGAGCCATCAGAAGCAGAG | | | | |
| | ATCTCCCACACCCAAAAGG | | | | |
| | CCACACTGGTATGCCTGGC | | | | |
| | CACAGGCTTCTACCCCGAC | | | | |
| | CACGTGGAGCTGAGCTGGT | | | | |
| | GGGTGAATGGGAAGGAGG | | | | |
| | TGCACAGTGGGGTCAGCAC | | | | |
| | AGACCGCAGCCCCTCAAG | | | | |
| | GAGCAGCCCGCCCTCAATG | | | | |
| | ACTCCAGATACTGCCTGAG | | | | |
| | CAGCCGCCTGAGGGTCTCG | | | | |
| | GCCACCTTCTGGCAGAACC | | | | |
| | CCCGCAACCACTTCCGCTG | | | | |
| | TCAAGTCCAGTTCTACGGG | | | | |
| | CTCTCGGAGAATGACGAGT | | | | |
| | GGACCCAGGATAGGGCCA | | | | |
| | AACCTGTCACCCAGATCGT | | | | |
| | CAGCGCCGAGGCCTGGGG | | | | |
| | TAGAGCAGACTGTGGCTTC | | | | |
| | ACCTCCGAGTCTTACCAGC | | | | |
| | AAGGGGTCCTGTCTGCCAC | | | | |
| | CATCCTCTATGAGATCTTG | | | | |
| | CTAGGGAAGGCCACCTTGT | | | | |
| | ATGCCGTGCTGGTCAGTGC | | | | |
| | CCTCGTGCTGATGGCCATG | | | | |
| | GTCAAGAGAAAGGATTCCA | | | | |
| | GAGGCTAG | | | | |
| | (SEQ ID NO: 260) | | | | |
| 5 | TGGGGGCAATGCCCAAAACC | MGSRLLCWVL | AAGGCTGGAG | KAGVTQTPRYL | TCTG |
| | CCAGCTCTCAGAGGACCAGT | LCLLGAGPVKA | TCACTCAAACT | IKTRGQQVTLS | GGCA |
| | ATCCCTCACAGGGTGACACC | GVTQTPRYLIK | CCAAGATATCT | CSPISGHRSVSW | TAGG |
| | TGACCAGCTCTGTCCCACCT | TRGQQVTLSCS | GATCAAAACG | YQQTPGQGLQF | AGT |
| | GGCCATGGGCTCCAGGTACC | PISGHRSVSWY | AGAGGACAGC | LFEYFSETQRN | (SEQ |
| | TCTGATGGGAAGACCTTTGT | QQTPGQGLQFL | AAGTGACACT | KGNFPGRFSGR | ID NO: |
| | CTCTTGGGAACAAGTGAATC | FEYFSETQRNK | GAGCTGCTCCC | QFSNSRSEMNV | 268) |
| | CTTGGCACAGGCCCAGTGGA | GNFPGRFSGRQ | CTATCTCTGGG | STLELGDSALY | |
| | TTCTGCTGTGCAGAACAGAG | FSNSRSEMNVS | CATAGGAGTGT | LCASRQRTELE | |
| | AGCAGTGGACCTCAGGAGGC | TLELGDSALYL | ATCCTGGTACC | AFFGQGTRLTV | |
| | CTGCAAGGGGAGGACATAG | CASRQRTELEA | AACAGACCCC | V | |
| | GACAGTGACATCACAGTATG | FFGQGTRLTVV | AGGACAGGGC | (SEQ ID NO: | |
| | CCCCTCCCACCAGGAAAAGC | EDLNKVFPPEV | CTTCAGTTCCT | 593) | |
| | AAGGCTGAGAATTTAGCTCT | AVFEPSEAEIS | CTTTGAATACT | | |
| | TTCCCAGGAGGACCAAGCCC | HTQKATLVCL | TCAGTGAGAC | | |
| | TGAGCACAGACACAGTGCTG | ATGFYPDHVE | ACAGAGAAAC | | |
| | CCTGCCCCTTTGTGCCATGG | LSWWVNGKE | AAAGGAAACT | | |
| | GCTCCAGGCTGCTCTGTTGG | VHSGVSTDPQP | TCCCTGGTCGA | | |
| | GTGCTGCTTTGTCTCCTGGGA | LKEQPALNDS | TTCTCAGGGCG | | |
| | GCAGGCCCAGTAAAGGCTGG | RYCLSSRLRVS | CCAGTTCTCTA | | |
| | AGTCACTCAAACTCCAAGAT | ATFWQNPRNH | ACTCTCGCTCT | | |
| | ATCTGATCAAAACGAGAGGA | FRCQVQFYGL | GAGATGAATG | | |
| | CAGCAAGTGACACTGAGCTG | SENDEWTQDR | TGAGCACCTTG | | |
| | CTCCCCTATCTCTGGGCATA | AKPVTQIVSAE | GAGCTGGGGG | | |
| | GGAGTGTATCCTGGTACCAA | AWGRADCGFT | ACTCGGCCCTT | | |
| | CAGACCCCAGGACAGGGCCT | SVSYQQGVLSA | TATCTTTGCGC | | |
| | TCAGTTCCTCTTTGAATACTT | TILYEILLGKA | CAGCAGGCAG | | |
| | CAGTGAGACACAGAGAAAC | TLYAVLVSALV | CGGACAGAAC | | |
| | AAAGGAAACTTCCCTGGTCG | LMAMVKRKD | TTGAAGCTTTC | | |
| | ATTCTCAGGGCGCCAGTTCT | F | TTTGGACAAGG | | |
| | CTAACTCTCGCTCTGAGATG | (SEQ ID NO: | CACCAGACTCA | | |
| | AATGTGAGCACCTTGGAGCT | 591) | CAGTTGTAG | | |
| | GGGGGACTCGGCCCTTTATC | | (SEQ ID NO: | | |
| | TTTGCGCCAGCAGGCAGCGG | | 592) | | |
| | ACAGAACTTGAAGCTTTCTT | | | | |
| | TGGACAAGGCACCAGACTCA | | | | |
| | CAGTTGTAGAGGACCTGAA | | | | |
| | CAAGGTGTTCCCACCCGAG | | | | |
| | GTCGCTGTGTTTGAGCCAT | | | | |
| | CAGAAGCAGAGATCTCCCA | | | | |
| | CACCCAAAAGGCCACACTG | | | | |
| | GTGTGCCTGGCCACAGGCT | | | | |
| | TCTACCCCGACCACGTGGA | | | | |
| | GCTGAGCTGGTGGGTGAAT | | | | |
| | GGGAAGGAGGTGCACAGT | | | | |
| | GGGGTCAGCACAGACCCG | | | | |
| | CAGCCCCTCAAGGAGCAGC | | | | |
| | CCGCCCTCAATGACTCCAG | | | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCCGTC | | | | |
| | ACCCAGATCGTCAGCGCCG | | | | |
| | AGGCCTGGGGTAGAGCAG | | | | |
| | ACTGTGGCTTTACCTCGGT | | | | |
| | GTCCTACCAGCAAGGGGTC | | | | |
| | CTGTCTGCCACCATCCTCT | | | | |
| | ATGAGATCCTGCTAGGGAA | | | | |
| | GGCCACCCTGTATGCTGTG | | | | |
| | CTGGTCAGCGCCCTTGTGT | | | | |
| | TGATGGCCATGGTCAAGAG | | | | |
| | AAAGGATTTCTGA | | | | |
| | (SEQ ID NO: 267) | | | | |
| 6 | GGCCAGAGACACCAGTAATT | MDTWLVCWAI | GAACCTGAAG | EPEVTQTPSHQ | TCTA |
| | CTGCCAGACCTTGCCTGTGG | FSLLKAGLTEPE | TCACCCAGACT | VTQMGQEVILR | ATCA |
| | GGCCATGGGAGCTCAAAATG | VTQTPSHQVTQ | CCCAGCCATCA | CVPISNHLYFY | CTTAT |
| | CCCCTCCTTTCCTCCACAGGA | MGQEVILRCVPI | GGTCACACAG | WYRQILGQKVE | AC |
| | CCAGATGCCTGAGCTAGGAA | SNHLYFYWYR | ATGGGACAGG | FLVSFYNNEISE | (SEQ |
| | AGGCCTCATTCCTGCTGTGA | QILGQKVEFLV | AAGTGATCTTG | KSEIFDDQFSVE | ID NO: |
| | TCCTGCCATGGATACCTGGC | SFYNNEISEKSE | CGCTGTGTCCC | RPDGSNFTLKIR | 275) |
| | TCGTATGCTGGGCAATTTTTA | IFDDQFSVERPD | CATCTCTAATC | STKLEDSAMYF | |
| | GTCTCTTGAAAGCAGGACTC | GSNFTLKIRSTK | ACTTATACTTC | CASSEAPDRGT | |
| | ACAGAACCTGAAGTCACCCA | LEDSAMYFCAS | TATTGGTACAG | YEQYFGPGTRL | |
| | GACTCCCAGCCATCAGGTCA | SEAPDRGTYEQ | ACAAATCTTGG | TVT | |
| | CACAGATGGGACAGGAAGT | YFGPGTRLTVT | GGCAGAAAGT | (SEQ ID NO: | |
| | GATCTTGCGCTGTGTCCCCAT | EDLKNVFPPEV | CGAGTTTCTGG | 596) | |
| | CTCTAATCACTTATACTTCTA | AVFEPSEAEIS | TTTCCTTTTAT | | |
| | TTGGTACAGACAAATCTTGG | HTQKATLVCL | AATAATGAAA | | |
| | GGCAGAAAGTCGAGTTTCTG | ATGFYPDHVE | TCTCAGAGAA | | |
| | GTTTCCTTTTATAATAATGAA | LSWWVNGKE | GTCTGAAATAT | | |
| | ATCTCAGAGAAGTCTGAAAT | VHSGVSTDPQP | TCGATGATCAA | | |
| | ATTCGATGATCAATTCTCAG | LKEQPALNDS | TTCTCAGTTGA | | |
| | TTGAAAGGCCTGATGGATCA | RYCLSSRLRVS | AAGGCCTGAT | | |
| | AATTTCACTCTGAAGATCCG | ATFWQNPRNH | GGATCAAATTT | | |
| | GTCCACAAAGCTGGAGGACT | FRCQVQFYGL | CACTCTGAAGA | | |
| | CAGCCATGTACTTCTGTGCC | SENDEWTQDR | TCCGGTCCACA | | |
| | AGCAGTGAAGCCCCTGACAG | AKPVTQIVSAE | AAGCTGGAGG | | |
| | GGGTACCTACGAGCAGTACT | AWGRADCGFT | ACTCAGCCATG | | |
| | TCGGGCCGGGCACCAGGCTC | SESYQQGVLSA | TACTTCTGTGC | | |
| | ACGGTCACAGAGGACCTGA | TILYEILLGKA | CAGCAGTGAA | | |
| | AAAACGTGTTCCCACCCGA | TLYAVLVSALV | GCCCCTGACAG | | |
| | GGTCGCTGTGTTTGAGCCA | LMAMVKRKDS | GGGTACCTACG | | |
| | TCAGAAGCAGAGATCTCCC | RG | AGCAGTACTTC | | |
| | ACACCCAAAAGGCCACACT | (SEQ ID NO: | GGGCCGGGCA | | |
| | GGTGTGCCTGGCCACAGGC | 594) | CCAGGCTCACG | | |
| | TTCTACCCCGACCACGTGG | | GTCACAG | | |
| | AGCTGAGCTGGTGGGTGAA | | (SEQ ID NO: | | |
| | TGGGAAGGAGGTGCACAG | | 595) | | |
| | TGGGGTCAGCACAGACCCG | | | | |
| | CAGCCCCTCAAGGAGCAGC | | | | |
| | CCGCCCTCAATGACTCCAG | | | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCTGTC | | | | |
| | ACCCAGATCGTCAGCGCCG | | | | |
| | AGGCCTGGGGTAGAGCAG | | | | |
| | ACTGTGGCTTCACCTCCGA | | | | |
| | GTCTTACCAGCAAGGGGTC | | | | |
| | CTGTCTGCCACCATCCTCT | | | | |
| | ATGAGATCTTGCTAGGGAA | | | | |
| | GGCCACCTTGTATGCCGTG | | | | |
| | CTGGTCAGTGCCCTCGTGC | | | | |
| | TGATGGCCATGGTCAAGAG | | | | |
| | AAAGGATTCCAGAGGCTAG | | | | |
| | (SEQ ID NO: 274) | | | | |
| 7 | GGGGACTGAGAGCTCAACTT | MGTRLFFYVAL | GATGCTGGAAT | DAGITQSPRYKI | TGGA |
| | CAATTTGCCCACAGCAGGGC | CLLWAGHRDA | CACCCAGAGC | TETGRQVTLMC | GCCA |
| | TGGGAGACACAAGATCCTGC | GITQSPRYKITE | CCAAGATACA | HQTWSHSYMF | CAGC |
| | CCTGGAGCTGAAATGGGCAC | TGRQVTLMCH | AGATCACAGA | WYRQDLGHGL | TAT |
| | CAGGCTCTTCTTCTATGTGGC | QTWSHSYMFW | GACAGGAAGG | RLIYYSAAADIT | (SEQ |
| | CCTTTGTCTGCTGTGGGCAG | YRQDLGHGLRL | CAGGTGACCTT | DKGEVPDGYV | ID NO: |
| | GACACAGGGATGCTGGAATC | IYYSAAADITD | GATGTGTCACC | VSRSKTENFPLT | 282) |
| | ACCCAGAGCCCAAGATACAA | KGEVPDGYVVS | AGACTTGGAG | LESATRSQTSV | |
| | GATCACAGAGACAGGAAGG | RSKTENFPLTLE | CCACAGCTATA | YFCASSELGRG | |
| | CAGGTGACCTTGATGTGTCA | SATRSQTSVYF | TGTTCTGGTAT | FYEQYFGPGTR | |
| | CCAGACTTGGAGCCACAGCT | CASSELGRGFY | CGACAAGACC | LTVT | |
| | ATATGTTCTGGTATCGACAA | EQYFGPGTRLT | TGGGACATGG | (SEQ ID NO: | |
| | GACCTGGGACATGGGCTGAG | VTEDLKNVFPP | GCTGAGGCTG | 599) | |
| | GCTGATCTATTACTCAGCAG | EVAVFEPSEAE | ATCTATTACTC | | |
| | CTGCTGATATTACAGATAAA | ISHTQKATLVC | AGCAGCTGCTG | | |
| | GGAGAAGTCCCCGATGGCTA | LATGFYPDHV | ATATTACAGAT | | |
| | CGTTGTCTCCAGATCCAAGA | ELSWWVNGKE | AAAGGAGAAG | | |
| | CAGAGAATTTCCCCCTCACT | VHSGVSTDPQP | TCCCCGATGGC | | |
| | CTGGAGTCAGCTACCCGCTC | LKEQPALNDS | TACGTTGTCTC | | |
| | CCAGACATCTGTGTATTTCTG | RYCLSSRLRVS | CAGATCCAAG | | |
| | CGCCAGCAGTGAGTTGGGCA | ATFWQNPRNH | ACAGAGAATTT | | |
| | GGGGTTTCTACGAGCAGTAC | FRCQVQFYGL | CCCCCTCACTC | | |
| | TTCGGGCCGGGCACCAGGCT | SENDEWTQDR | TGGAGTCAGCT | | |
| | CACGGTCACAGAGGACCTG | AKPVTQIVSAE | ACCCGCTCCCA | | |
| | AAAAACGTGTTCCCACCCG | AWGRADCGFT | GACATCTGTGT | | |
| | AGGTCGCTGTGTTTGAGCC | SESYQQGVLSA | ATTTCTGCGCC | | |
| | ATCAGAAGCAGAGATCTCC | TILYEILLGKA | AGCAGTGAGTT | | |
| | CACACCCAAAAGGCCACAC | TLYAVLVSALV | GGGCAGGGGT | | |
| | TGGTGTGCCTGGCCACAGG | LMAMVKRKDS | TTCTACGAGCA | | |
| | CTTCTACCCCGACCACGTG | RG | GTACTTCGGGC | | |
| | GAGCTGAGCTGGTGGGTG | (SEQ ID NO: | CGGGCACCAG | | |
| | AATGGGAAGGAGGTGCAC | 597) | GCTCACGGTCA | | |
| | AGTGGGGTCAGCACAGACC | | CAG | | |
| | CGCAGCCCCTCAAGGAGCA | | (SEQ ID NO: | | |
| | GCCCGCCCTCAATGACTCC | | 598) | | |
| | AGATACTGCCTGAGCAGCC | | | | |
| | GCCTGAGGGTCTCGGCCAC | | | | |
| | CTTCTGGCAGAACCCCCGC | | | | |
| | AACCACTTCCGCTGTCAAG | | | | |
| | TCCAGTTCTACGGGCTCTC | | | | |
| | GGAGAATGACGAGTGGAC | | | | |
| | CCAGGATAGGGCCAAACCT | | | | |
| | GTCACCCAGATCGTCAGCG | | | | |
| | CCGAGGCCTGGGGTAGAG | | | | |
| | CAGACTGTGGCTTCACCTC | | | | |
| | CGAGTCTTACCAGCAAGGG | | | | |
| | GTCCTGTCTGCCACCATCC | | | | |
| | TCTATGAGATCTTGCTAGG | | | | |
| | GAAGGCCACCTTGTATGCC | | | | |
| | GTGCTGGTCAGTGCCCTCG | | | | |
| | TGCTGATGGCCATGGTCAA | | | | |
| | GAGAAAGGATTCCAGAGGC | | | | |
| | TAG | | | | |
| | (SEQ ID NO: 281) | | | | |
| 8 | GATGGAAAGCAAGAGCCCTG | MGVASPGLSLP | AATGCTGGTGT | NAGVTQTPKFQ | ATGA |
| | GGTGGAGCTGAAGGTGCTCA | GAQAGRMSQN | CACTCAGACCC | VLKTGQSMTLQ | ACCA |
| | GCTGGGTTTGTCAGAAGTCT | DFLESPAPLSSM | CAAAATTCCAG | CAQDMNHEYM | TGAA |
| | CATCTGTCACTGTTCACTGGT | HRYRRPLRHAA | GTCCTGAAGAC | SWYRQDPGMG | TAC |
| | GCATTTATTTTGGATTTGACC | SAMSIGLLCCA | AGGACAGAGC | LRLIHYSVGAGI | (SEQ |
| | ATCTGGGGAATGGGTGTGGC | ALSLLWAGPVN | ATGACACTGCA | TDQGEVPNGYN | ID NO: |
| | CTCTCCTGGCCTCTCCCTCCC | AGVTQTPKFQV | GTGTGCCCAGG | VSRSTTEDFPLR | 289) |
| | TGGGGCCCAGGCAGGGAGA | LKTGQSMTLQC | ATATGAACCAT | LLSAAPSQTSV | |
| | ATGTCTCAGAATGACTTCCTT | AQDMNHEYMS | GAATACATGTC | YFCASSHLGAG | |
| | GAGAGTCCTGCTCCCCTTTC | WYRQDPGMGL | CTGGTATCGAC | GPHEQYFGPGT | |
| | ATCAATGCACAGATACAGAA | RLIHYSVGAGIT | AAGACCCAGG | RLTVT | |
| | GACCCCTCCGTCATGCAGCA | DQGEVPNGYN | CATGGGGCTG | (SEQ ID NO: | |
| | TCTGCCATGAGCATCGGCCT | VSRSTTEDFPLR | AGGCTGATTCA | 602) | |
| | CCTGTGCTGTGCAGCCTTGTC | LLSAAPSQTSV | TTACTCAGTTG | | |
| | TCTCCTGTGGGCAGGTCCAG | YFCASSHLGAG | GTGCTGGTATC | | |
| | TGAATGCTGGTGTCACTCAG | GPHEQYFGPGT | ACTGACCAAG | | |
| | ACCCCAAAATTCCAGGTCCT | RLTVTEDLKNV | GAGAAGTCCC | | |
| | GAAGACAGGACAGAGCATG | FPPEVAVFEPS | CAATGGCTACA | | |
| | ACACTGCAGTGTGCCCAGGA | EAEISHTQKAT | ATGTCTCCAGA | | |
| | TATGAACCATGAATACATGT | LVCLATGFYP | TCAACCACAG | | |
| | CCTGGTATCGACAAGACCCA | DHVELSWWVN | AGGATTTCCCG | | |
| | GGCATGGGGCTGAGGCTGAT | GKEVHSGVST | CTCAGGCTGCT | | |
| | TCATTACTCAGTTGGTGCTG | DPQPLKEQPA | GTCGGCTGCTC | | |
| | GTATCACTGACCAAGGAGAA | LNDSRYCLSSR | CCTCCCAGACA | | |
| | GTCCCCAATGGCTACAATGT | LRVSATFWQN | TCTGTGTACTT | | |
| | CTCCAGATCAACCACAGAGG | PRNHFRCQVQ | CTGTGCCAGCA | | |
| | ATTTCCCGCTCAGGCTGCTGT | FYGLSENDEW | GCCATCTGGGG | | |
| | CGGCTGCTCCCTCCCAGACA | TQDRAKPVTQI | GCGGGAGGGC | | |
| | TCTGTGTACTTCTGTGCCAGC | VSAEAWGRAD | CGCACGAGCA | | |
| | AGCCATCTGGGGGCGGGAGG | CGFTSESYQQ | GTACTTCGGGC | | |
| | GCCGCACGAGCAGTACTTCG | GVLSATILYEI | CGGGCACCAG | | |
| | GGCCGGGCACCAGGCTCACG | LLGKATLYAV | GCTCACGGTCA | | |
| | GTCACAGAGGACCTGAAAA | LVSALVLMAM | CAG | | |
| | ACGTGTTCCCACCCGAGGT | VKRKDSRG | (SEQ ID NO: | | |
| | CGCTGTGTTTGAGCCATCA | (SEQ ID NO: | 601) | | |
| | GAAGCAGAGATCTCCCACA | 600) | | | |
| | CCCAAAAGGCCACACTGGT | | | | |
| | GTGCCTGGCCACAGGCTTC | | | | |
| | TACCCCGACCACGTGGAGC | | | | |
| | TGAGCTGGTGGGTGAATGG | | | | |
| | GAAGGAGGTGCACAGTGG | | | | |
| | GGTCAGCACAGACCCGCAG | | | | |
| | CCCCTCAAGGAGCAGCCCG | | | | |
| | CCCTCAATGACTCCAGATA | | | | |
| | CTGCCTGAGCAGCCGCCTG | | | | |
| | AGGGTCTCGGCCACCTTCT | | | | |
| | GGCAGAACCCCCGCAACCA | | | | |
| | CTTCCGCTGTCAAGTCCAG | | | | |
| | TTCTACGGGCTCTCGGAGA | | | | |
| | ATGACGAGTGGACCCAGGA | | | | |
| | TAGGGCCAAACCTGTCACC | | | | |
| | CAGATCGTCAGCGCCGAGG | | | | |
| | CCTGGGGTAGAGCAGACTG | | | | |
| | TGGCTTCACCTCCGAGTCT | | | | |
| | TACCAGCAAGGGGTCCTGT | | | | |
| | CTGCCACCATCCTCTATGA | | | | |
| | GATCTTGCTAGGGAAGGCC | | | | |
| | ACCTTGTATGCCGTGCTGG | | | | |
| | TCAGTGCCCTCGTGCTGAT | | | | |
| | GGCCATGGTCAAGAGAAAG | | | | |
| | GATTCCAGAGGCTAG | | | | |
| | (SEQ ID NO: 288) | | | | |
| 9 | GGTGACTGGGGGAATGGAG | MGTSLLCWVV | GGTGCTGGAGT | GAGVSQSPRYK | TCGG |
| | GAGGCTGGAGCATGAATGGG | LGFLGTDHTGA | CTCCCAGTCTC | VTKRGQDVAL | GTCA |
| | GATGGCACTGGGGACCCTGA | GVSQSPRYKVT | CCAGGTACAA | RCDPISGHVSLY | TGTA |
| | CTTGCAGGAAATGCAATGAG | KRGQDVALRC | AGTCACAAAG | WYRQALGQGP | TCC |
| | CTCACCACTTTGTGCCCTATG | DPISGHVSLYW | AGGGGACAGG | EFLTYFNYEAQ | (SEQ |
| | TTAGGGGCGGTGTTGGTGCA | YRQALGQGPEF | ATGTAGCTCTC | QDKSGLPNDRF | ID NO: |
| | TCCTAGAGTAAATGTCCAGC | LTYFNYEAQQD | AGGTGTGATCC | SAERPEGSISTL | 296) |
| | AGACAAAGGAGCAAGAAGC | KSGLPNDRFSA | AATTTCGGGTC | TIQRTEQRDSA | |
| | GGCTGTGGGATGACAAGATA | ERPEGSISTLTIQ | ATGTATCCCTT | MYRCASSHTDG | |
| | AACTCAGAGATACAGCATGA | RTEQRDSAMYR | TATTGGTACCG | SYEQYFGPGTR | |
| | GACCTCCGGGTCCAGACAGC | CASSHTDGSYE | ACAGGCCCTG | LTVT | |
| | TCTGGAGCCCAAGGCGATGA | QYFGPGTRLTV | GGGCAGGGCC | (SEQ ID NO: | |
| | GCCATGCATTGATGTTGTTA | TEDLKNVFPPE | CAGAGTTTCTG | 605) | |
| | AAAAGGAGCTGATAAATATT | VAVFEPSEAEI | ACTTACTTCAA | | |
| | TAAAGCAGCACCCAACTGTG | SHTQKATLVC | TTATGAAGCCC | | |
| | TTCTAATAGAAATGCTGTGA | LATGFYPDHV | AACAAGACAA | | |
| | TCCTGAGGTCCTGGGGATTG | ELSWWVNGKE | ATCAGGGCTGC | | |
| | AGAGAGGAAGTGATGTCACT | VHSGVSTDPQP | CCAATGATCGG | | |
| | GTGGGAACTGCCCTGTGGAG | LKEQPALNDS | TTCTCTGCAGA | | |
| | ACAAGGACATCCCTCATCCT | RYCLSSRLRVS | GAGGCCTGAG | | |
| | CTGCTGCTGCTCACAGTGAC | ATFWQNPRNH | GGATCCATCTC | | |
| | ACTGATCTGGTAAAGCCCTC | FRCQVQFYGL | CACTCTGACGA | | |
| | ATCCTGTCCTGACCCTGCCAT | SENDEWTQDR | TCCAGCGCACA | | |
| | GGGCACCAGTCTCCTATGCT | AKPVTQIVSAE | GAGCAGCGGG | | |
| | GGGTGGTCCTGGGTTTCCTA | AWGRADCGFT | ACTCGGCCATG | | |
| | GGGACAGATCACACAGGTGC | SESYQQGVLSA | TATCGCTGTGC | | |
| | TGGAGTCTCCCAGTCTCCCA | TILYEILLGKA | CAGCAGCCAT | | |
| | GGTACAAAGTCACAAAGAG | TLYAVLVSALV | ACTGACGGCTC | | |
| | GGGACAGGATGTAGCTCTCA | LMAMVKRKDS | CTACGAGCAGT | | |
| | GGTGTGATCCAATTTCGGGT | RG | ACTTCGGGCCG | | |
| | CATGTATCCCTTTATTGGTAC | (SEQ ID NO: | GGCACCAGGC | | |
| | CGACAGGCCCTGGGGCAGGG | 603) | TCACGGTCACA | | |
| | CCCAGAGTTTCTGACTTACTT | | G | | |
| | CAATTATGAAGCCCAACAAG | | (SEQ ID NO: | | |
| | ACAAATCAGGGCTGCCCAAT | | 604) | | |
| | GATCGGTTCTCTGCAGAGAG | | | | |
| | GCCTGAGGGATCCATCTCCA | | | | |
| | CTCTGACGATCCAGCGCACA | | | | |
| | GAGCAGCGGGACTCGGCCAT | | | | |
| | GTATCGCTGTGCCAGCAGCC | | | | |
| | ATACTGACGGCTCCTACGAG | | | | |
| | CAGTACTTCGGGCCGGGCAC | | | | |
| | CAGGCTCACGGTCACAGAGG | | | | |
| | ACCTGAAAAACGTGTTCCC | | | | |
| | ACCCGAGGTCGCTGTGTTT | | | | |
| | GAGCCATCAGAAGCAGAGA | | | | |
| | TCTCCCACACCCAAAAGGC | | | | |
| | CACACTGGTATGCCTGGCC | | | | |
| | ACAGGCTTCTACCCCGACC | | | | |
| | ACGTGGAGCTGAGCTGGTG | | | | |
| | GGTGAATGGGAAGGAGGT | | | | |
| | GCACAGTGGGGTCAGCACA | | | | |
| | GACCCGCAGCCCCTCAAGG | | | | |
| | AGCAGCCCGCCCTCAATGA | | | | |
| | CTCCAGATACTGCCTGAGC | | | | |
| | AGCCGCCTGAGGGTCTCGG | | | | |
| | CCACCTTCTGGCAGAACCC | | | | |
| | CCGCAACCACTTCCGCTGT | | | | |
| | CAAGTCCAGTTCTACGGGC | | | | |
| | TCTCGGAGAATGACGAGTG | | | | |
| | GACCCAGGATAGGGCCAAA | | | | |
| | CCTGTCACCCAGATCGTCA | | | | |
| | GCGCCGAGGCCTGGGGTA | | | | |
| | GAGCAGACTGTGGCTTCAC | | | | |
| | CTCCGAGTCTTACCAGCAA | | | | |
| | GGGGTCCTGTCTGCCACCA | | | | |
| | TCCTCTATGAGATCTTGCT | | | | |
| | AGGGAAGGCCACCTTGTAT | | | | |
| | GCCGTGCTGGTCAGTGCCC | | | | |
| | TCGTGCTGATGGCCATGGT | | | | |
| | CAAGAGAAAGGATTCCAGA | | | | |
| | GGCTAG (SEQ ID NO: 295) | | | | |
| 10 | TGGGGACAGATGCAATGCAG | MSNQVLCCVV | GATGGTGGAA | DGGITQSPKYLF | TTGA |
| | AGTTATGGGAGGTGCGAATG | LCLLGANTVDG | TCACTCAGTCC | RKEGQNVTLSC | ACCA |
| | ACTCTGCTCTCTGTCCTGTCT | GITQSPKYLFRK | CCAAAGTACCT | EQNLNHDAMY | |
| | CCTCATCTGCAAAATTAGGA | EGQNVTLSCEQ | GTTCAGAAAG | WYRQDPGQGL | CGAT |
| | AGCCTGTCTTGATTATCTCCA | NLNHDAMYWY | GAAGGACAGA | RLIYYSQIVNDF | GCC |
| | GGAACCTCCCACCTCTTCATT | RQDPGQGLRLI | ATGTGACCCTG | QKGDIAEGYSV | (SEQ |
| | CCAGCCTCTGACAAACTCTG | YYSQIVNDFQK | AGTTGTGAACA | SREKKESFPLTV | ID NO: |
| | CACATTAGGCCAGGAGAAGC | GDIAEGYSVSR | GAATTTGAACC | TSAQKNPTAFY | 303) |
| | CCCCGAGCCAAGTCTCTTTTC | EKKESFPLTVTS | ACGATGCCATG | LCASSIGAFAG | |
| | TCATTCTCTTCCAACAAGTGC | AQKNPTAFYLC | TACTGGTACCG | QPQHFGDGTRL | |
| | TTGGAGCTCCAAGAAGGCCC | ASSIGAFAGQP | ACAGGACCCA | SIL | |
| | CCTTTGCACTATGAGCAACC | QHFGDGTRLSIL | GGGCAAGGGC | (SEQ ID NO: | |
| | AGGTGCTCTGCTGTGTGGTC | EDLNKVFPPEV | TGAGATTGATC | 608) | |
| | CTTTGTCTCCTGGGAGCAAA | AVFEPSEAEIS | TACTACTCACA | | |
| | CACCGTGGATGGTGGAATCA | HTQKATLVCL | GATAGTAAAT | | |
| | CTCAGTCCCCAAAGTACCTG | ATGFYPDHVE | GACTTTCAGAA | | |
| | TTCAGAAAGGAAGGACAGA | LSWWVNGKE | AGGAGATATA | | |
| | ATGTGACCCTGAGTTGTGAA | VHSGVSTDPQP | GCTGAAGGGT | | |
| | CAGAATTTGAACCACGATGC | LKEQPALNDS | ACAGCGTCTCT | | |
| | CATGTACTGGTACCGACAGG | RYCL | CGGGAGAAGA | | |
| | ACCCAGGGCAAGGGCTGAG | (SEQ ID NO: | AGGAATCCTTT | | |
| | ATTGATCTACTACTCACAGA | 506) | CCTCTCACTGT | | |
| | TAGTAAATGACTTTCAGAAA | | GACATCGGCCC | | |
| | GGAGATATAGCTGAAGGGTA | | AAAAGAACCC | | |
| | CAGCGTCTCTCGGGAGAAGA | | GACAGCTTTCT | | |
| | AGGAATCCTTTCCTCTCACTG | | ATCTCTGTGCC | | |
| | TGACATCGGCCCAAAAGAAC | | AGTAGCATAG | | |
| | CCGACAGCTTTCTATCTCTGT | | GGGCATTTGCT | | |
| | GCCAGTAGCATAGGGGCATT | | GGTCAGCCCCA | | |
| | TGCTGGTCAGCCCCAGCATT | | GCATTTTGGTG | | |
| | TTGGTGATGGGACTCGACTC | | ATGGGACTCG | | |
| | TCCATCCTAGAGGACCTGAA | | ACTCTCCATCC | | |
| | CAAGGTGTTCCCACCCGAG | | TAG | | |
| | GTCGCTGTGTTTGAGCCAT | | (SEQ ID NO: | | |
| | CAGAAGCAGAGATCTCCCA | | 607) | | |
| | CACCCAAAAGGCCACACTG | | | | |
| | GTGTGCCTGGCCACAGGCT | | | | |
| | TCTACCCCGACCACGTGGA | | | | |
| | GCTGAGCTGGTGGGTGAAT | | | | |
| | GGGAAGGAGGTGCACAGT | | | | |
| | GGGGTCAGCACAGACCCG | | | | |
| | CAGCCCCTCAAGGAGCAGC | | | | |
| | CCGCCCTCAATGACTCCAG | | | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCCGTC | | | | |
| | ACCCAGATCGTCAGCGCCG | | | | |
| | AGGCCTGGGGTAGAGCAG | | | | |
| | ACTGTGGCTTTACCTCGGT | | | | |
| | GTCCTACCAGCAAGGGGTC | | | | |
| | CTGTCTGCCACCATCCTCT | | | | |
| | ATGAGATCCTGCTAGGGAA | | | | |
| | GGCCACCCTGTATGCTGTG | | | | |
| | CTGGTCAGCGCCCTTGTGT | | | | |
| | TGATGGCCATGGTCAAGAG | | | | |
| | AAAGGATTTCTGA | | | | |
| | (SEQ ID NO: 302) | | | | |
| 11 | TGGGGGTCACTGTGGGAACT | MGTSLLCWMA | GATACTGGAGT | DTGVSQNPRHK | TCTG |
| | GCTCTGTGGCGACAAGGACG | LCLLGADHADT | CTCCCAGAACC | ITKRGQNVTFR | AACA |
| | TCCCTCATCCTCTGCTCCTGC | GVSQNPRHKIT | CCAGACACAA | CDPISEHNRLY | CAAC |
| | TCACAGTGACCCTGATCTGG | KRGQNVTFRCD | GATCACAAAG | WYRQTLGQGP | CGC |
| | TAAAGCTCCCATCCTGCCCT | PISEHNRLYWY | AGGGGACAGA | EFLTYFQNEAQ | (SEQ |
| | GACCCTGCCATGGGCACCAG | RQTLGQGPEFL | ATGTAACTTTC | LEKSRLLSDRFS | ID NO: |
| | CCTCCTCTGCTGGATGGCCCT | TYFQNEAQLEK | AGGTGTGATCC | AERPKGSFSTLE | 310) |
| | GTGTCTCCTGGGGGCAGATC | SRLLSDRFSAER | AATTTCTGAAC | IQRTEQGDSAM | |
| | ACGCAGATACTGGAGTCTCC | PKGSFSTLEIQR | ACAACCGCCTT | YLCASSLAPTP | |
| | CAGAACCCCAGACACAAGAT | TEQGDSAMYLC | TATTGGTACCG | GPDTGELFFGE | |
| | CACAAAGAGGGGACAGAAT | ASSLAPTPGPDT | ACAGACCCTG | GSRLTVL | |
| | GTAACTTTCAGGTGTGATCC | GELFFGEGSRLT | GGGCAGGGCC | (SEQ ID NO: | |
| | AATTTCTGAACACAACCGCC | VLEDLKNVFPP | CAGAGTTTCTG | 611) | |
| | TTTATTGGTACCGACAGACC | EVAVFEPSEAE | ACTTACTTCCA | | |
| | CTGGGGCAGGGCCCAGAGTT | ISHTQKATLVC | GAATGAAGCT | | |
| | TCTGACTTACTTCCAGAATG | LATGFYPDHV | CAACTAGAAA | | |
| | AAGCTCAACTAGAAAAATCA | ELSWWVNGKE | AATCAAGGCT | | |
| | AGGCTGCTCAGTGATCGGTT | VHSGVSTDPQP | GCTCAGTGATC | | |
| | CTCTGCAGAGAGGCCTAAGG | LKEQPALNDS | GGTTCTCTGCA | | |
| | GATCTTTCTCCACCTTGGAG | RYCLSSRLRVS | GAGAGGCCTA | | |
| | ATCCAGCGCACAGAGCAGGG | ATFWQNPRNH | AGGGATCTTTC | | |
| | GGACTCGGCCATGTATCTCT | FRCQVQFYGL | TCCACCTTGGA | | |
| | GTGCCAGCAGCTTAGCGCCT | SENDEWTQDR | GATCCAGCGC | | |
| | ACCCCCGGGCCGGACACCGG | AKPVTQIVSAE | ACAGAGCAGG | | |
| | GGAGCTGTTTTTTGGAGAAG | AWGRADCGFT | GGGACTCGGC | | |
| | GCTCTAGGCTGACCGTACTG | SESYQQGVLSA | CATGTATCTCT | | |
| | GAGGACCTGAAAAACGTGT | TILYEILLGKA | GTGCCAGCAG | | |
| | TCCCACCCGAGGTCGCTGT | TLYAVLVSALV | CTTAGCGCCTA | | |
| | GTTTGAGCCATCAGAAGCA | LMAMVKRKDS | CCCCCGGGCCG | | |
| | GAGATCTCCCACACCCAAA | RG | GACACCGGGG | | |
| | AGGCCACACTGGTGTGCCT | (SEQ ID NO: | AGCTGTTTTTT | | |
| | GGCCACAGGCTTCTACCCC | 609) | GGAGAAGGCT | | |
| | GACCACGTGGAGCTGAGCT | | CTAGGCTGACC | | |
| | GGTGGGTGAATGGGAAGG | | GTACTGG | | |
| | AGGTGCACAGTGGGGTCA | | (SEQ ID NO: | | |
| | GCACAGACCCGCAGCCCCT | | 610) | | |
| | CAAGGAGCAGCCCGCCCTC | | | | |
| | AATGACTCCAGATACTGCC | | | | |
| | TGAGCAGCCGCCTGAGGGT | | | | |
| | CTCGGCCACCTTCTGGCAG | | | | |
| | AACCCCCGCAACCACTTCC | | | | |
| | GCTGTCAAGTCCAGTTCTA | | | | |
| | CGGGCTCTCGGAGAATGAC | | | | |
| | GAGTGGACCCAGGATAGG | | | | |
| | GCCAAACCTGTCACCCAGA | | | | |
| | TCGTCAGCGCCGAGGCCTG | | | | |
| | GGGTAGAGCAGACTGTGG | | | | |
| | CTTCACCTCCGAGTCTTAC | | | | |
| | CAGCAAGGGGTCCTGTCTG | | | | |
| | CCACCATCCTCTATGAGAT | | | | |
| | CTTGCTAGGGAAGGCCACC | | | | |
| | TTGTATGCCGTGCTGGTCA | | | | |
| | GTGCCCTCGTGCTGATGGC | | | | |
| | CATGGTCAAGAGAAAGGAT | | | | |
| | TCCAGAGGCTAG (SEQ ID | | | | |
| | NO: 309) | | | | |
| 12 | GGCGTTTGTACACTTCAGCC | MGPQLLGYVV | GAAGCCCAAG | EAQVTQNPRYL | ATGA |
| | CTCTGAGCTGAAGTGGGAGT | LCLLGAGPLEA | TGACCCAGAA | ITVTGKKLTVT | ACCA |
| | GGTTTCTCTCCTGAAAATGTT | QVTQNPRYLIT | CCCAAGATACC | CSQNMNHEYM | TGAG |
| | TCTGAGGCCCAAATAGCTGA | VTGKKLTVTCS | TCATCACAGTG | SWYRQDPGLGL | TAT |
| | AGAGGTGGAGACGTTACAGA | QNMNHEYMSW | ACTGGAAAGA | RQIYYSMNVEV | (SEQ |
| | AACCACCTGGAGCCCCCAGA | YRQDPGLGLRQ | AGTTAACAGTG | TDKGDVPEGYK | ID NO: |
| | ACTGGCAGACACCTGCCTGA | IYYSMNVEVTD | ACTTGTTCTCA | VSRKEKRNFPLI | 317) |
| | TGCTGCCATGGGCCCCCAGC | KGDVPEGYKVS | GAATATGAAC | LESPSPNQTSLY | |
| | TCCTTGGCTATGTGGTCCTTT | RKEKRNFPLILE | CATGAGTATAT | FCASSLGNIYEQ | |
| | GCCTTCTAGGAGCAGGCCCC | SPSPNQTSLYFC | GTCCTGGTATC | YFGPGTRLTVT | |
| | CTGGAAGCCCAAGTGACCCA | ASSLGNIYEQYF | GACAAGACCC | (SEQ ID NO: | |
| | GAACCCAAGATACCTCATCA | GPGTRLTVTED | AGGGCTGGGC | 614) | |
| | CAGTGACTGGAAAGAAGTTA | LKNVFPPEVAV | TTAAGGCAGAT | | |
| | ACAGTGACTTGTTCTCAGAA | FEPSEAEISHT | CTACTATTCAA | | |
| | TATGAACCATGAGTATATGT | QKATLVCLAT | TGAATGTTGAG | | |
| | CCTGGTATCGACAAGACCCA | GFYPDHVELS | GTGACTGATAA | | |
| | GGGCTGGGCTTAAGGCAGAT | WWVNGKEVH | GGGAGATGTTC | | |
| | CTACTATTCAATGAATGTTG | SGVSTDPQPLK | CTGAAGGGTA | | |
| | AGGTGACTGATAAGGGAGAT | EQPALNDSRY | CAAAGTCTCTC | | |
| | GTTCCTGAAGGGTACAAAGT | CLSSRLRVSAT | GAAAAGAGAA | | |
| | CTCTCGAAAAGAGAAGAGG | FWQNPRNHFR | GAGGAATTTCC | | |
| | AATTTCCCCCTGATCCTGGA | CQVQFYGLSE | CCCTGATCCTG | | |
| | GTCGCCCAGCCCCAACCAGA | NDEWTQDRAK | GAGTCGCCCA | | |
| | CCTCTCTGTACTTCTGTGCCA | PVTQIVSAEAW | GCCCCAACCA | | |
| | GCAGTTTAGGGAATATCTAC | GRADCGFTSES | GACCTCTCTGT | | |
| | GAGCAGTACTTCGGGCCGGG | YQQGVLSATIL | ACTTCTGTGCC | | |
| | CACCAGGCTCACGGTCACAG | YEILLGKATLY | AGCAGTTTAGG | | |
| | AGGACCTGAAAAACGTGTT | AVLVSALVLM | GAATATCTACG | | |
| | CCCACCCGAGGTCGCTGTG | AMVKRKDSRG | AGCAGTACTTC | | |
| | TTTGAGCCATCAGAAGCAG | (SEQ ID NO: | GGGCCGGGCA | | |
| | AGATCTCCCACACCCAAAA | 612) | CCAGGCTCACG | | |
| | GGCCACACTGGTGTGCCTG | | GTCACAG | | |
| | GCCACAGGCTTCTACCCCG | | (SEQ ID NO: | | |
| | ACCACGTGGAGCTGAGCTG | | 613) | | |
| | GTGGGTGAATGGGAAGGA | | | | |
| | GGTGCACAGTGGGGTCAG | | | | |
| | CACAGACCCGCAGCCCCTC | | | | |
| | AAGGAGCAGCCCGCCCTCA | | | | |
| | ATGACTCCAGATACTGCCT | | | | |
| | GAGCAGCCGCCTGAGGGT | | | | |
| | CTCGGCCACCTTCTGGCAG | | | | |
| | AACCCCCGCAACCACTTCC | | | | |
| | GCTGTCAAGTCCAGTTCTA | | | | |
| | CGGGCTCTCGGAGAATGAC | | | | |
| | GAGTGGACCCAGGATAGG | | | | |
| | GCCAAACCTGTCACCCAGA | | | | |
| | TCGTCAGCGCCGAGGCCTG | | | | |
| | GGGTAGAGCAGACTGTGG | | | | |
| | CTTCACCTCCGAGTCTTAC | | | | |
| | CAGCAAGGGGTCCTGTCTG | | | | |
| | CCACCATCCTCTATGAGAT | | | | |
| | CTTGCTAGGGAAGGCCACC | | | | |
| | TTGTATGCCGTGCTGGTCA | | | | |
| | GTGCCCTCGTGCTGATGGC | | | | |
| | CATGGTCAAGAGAAAGGAT | | | | |
| | TCCAGAGGCTAG (SEQ ID | | | | |
| | NO: 316) | | | | |
| 13 | GAGAGAGGAAGTGATGTCAC | MGTRLLCWAA | GGTGCTGGAGT | GAGVSQTPSNK | TCAG |
| | TGTGGGTACTGTTCTGTGTCA | LCLLGADHTGA | CTCCCAGACCC | VTEKGKYVELR | GTCA |
| | GGACAAGGACGTCCCTCCTC | GVSQTPSNKVT | CCAGTAACAA | CDPISGHTALY | TACT |
| | CTCTGCTCCTGCTCACAGTG | EKGKYVELRCD | GGTCACAGAG | WYRQSLGQGPE | GCC |
| | ACCCTGATCTGGTAAAGCTC | PISGHTALYWY | AAGGGAAAAT | FLIYFQGTGAA | (SEQ |
| | CCATCCTGCCCTGACTCTGTC | RQSLGQGPEFLI | ATGTAGAGCTC | DDSGLPNDRFF | ID NO: |
| | ATGGGCACCAGGCTCCTCTG | YFQGTGAADDS | AGGTGTGATCC | AVRPEGSVSTL | 324) |
| | CTGGGCAGCCCTGTGCCTCC | GLPNDRFFAVR | AATTTCAGGTC | KIQRTERGDSA | |
| | TGGGGGCAGATCACACAGGT | PEGSVSTLKIQR | ATACTGCCCTT | VYLCASSLGSG | |
| | GCTGGAGTCTCCCAGACCCC | TERGDSAVYLC | TACTGGTACCG | EQFFGPGTRLT | |
| | CAGTAACAAGGTCACAGAGA | ASSLGSGEQFF | ACAAAGCCTG | VL | |
| | AGGGAAAATATGTAGAGCTC | GPGTRLTVLED | GGGCAGGGCC | (SEQ ID NO: | |
| | AGGTGTGATCCAATTTCAGG | LKNVFPPEVAV | CAGAGTTTCTA | 617) | |
| | TCATACTGCCCTTTACTGGTA | FEPSEAEISHT | ATTTACTTCCA | | |
| | CCGACAAAGCCTGGGGCAGG | QKATLVCLAT | AGGCACGGGT | | |
| | GCCCAGAGTTTCTAATTTACT | GFYPDHVELS | GCGGCAGATG | | |
| | TCCAAGGCACGGGTGCGGCA | WWVNGKEVH | ACTCAGGGCTG | | |
| | GATGACTCAGGGCTGCCCAA | SGVSTDPQPLK | CCCAACGATCG | | |
| | CGATCGGTTCTTTGCAGTCA | EQPALNDSRY | GTTCTTTGCAG | | |
| | GGCCTGAGGGATCCGTCTCT | CLSSRLRVSAT | TCAGGCCTGAG | | |
| | ACTCTGAAGATCCAGCGCAC | FWQNPRNHFR | GGATCCGTCTC | | |
| | AGAGCGGGGGGACTCAGCC | CQVQFYGLSE | TACTCTGAAGA | | |
| | GTGTATCTCTGTGCCAGCAG | NDEWTQDRAK | TCCAGCGCACA | | |
| | CTTAGGGAGCGGTGAGCAGT | PVTQIVSAEAW | GAGCGGGGGG | | |
| | TCTTCGGGCCAGGGACACGG | GRADCGFTSES | ACTCAGCCGTG | | |
| | CTCACCGTGCTAGAGGACCT | YQQGVLSATIL | TATCTCTGTGC | | |
| | GAAAAACGTGTTCCCACCC | YEILLGKATLY | CAGCAGCTTAG | | |
| | GAGGTCGCTGTGTTTGAGC | AVLVSALVLM | GGAGCGGTGA | | |
| | CATCAGAAGCAGAGATCTC | AMVKRKDSRG | GCAGTTCTTCG | | |
| | CCACACCCAAAAGGCCACA | (SEQ ID NO: | GGCCAGGGAC | | |
| | CTGGTGTGCCTGGCCACAG | 615) | ACGGCTCACCG | | |
| | GCTTCTACCCCGACCACGT | | TGCTAG | | |
| | GGAGCTGAGCTGGTGGGT | | (SEQ ID NO: | | |
| | GAATGGGAAGGAGGTGCA | | 616) | | |
| | CAGTGGGGTCAGCACAGAC | | | | |
| | CCGCAGCCCCTCAAGGAGC | | | | |
| | AGCCCGCCCTCAATGACTC | | | | |
| | CAGATACTGCCTGAGCAGC | | | | |
| | CGCCTGAGGGTCTCGGCCA | | | | |
| | CCTTCTGGCAGAACCCCCG | | | | |
| | CAACCACTTCCGCTGTCAA | | | | |
| | GTCCAGTTCTACGGGCTCT | | | | |
| | CGGAGAATGACGAGTGGA | | | | |
| | CCCAGGATAGGGCCAAACC | | | | |
| | TGTCACCCAGATCGTCAGC | | | | |
| | GCCGAGGCCTGGGGTAGA | | | | |
| | GCAGACTGTGGCTTCACCT | | | | |
| | CCGAGTCTTACCAGCAAGG | | | | |
| | GGTCCTGTCTGCCACCATC | | | | |
| | CTCTATGAGATCTTGCTAG | | | | |
| | GGAAGGCCACCTTGTATGC | | | | |
| | CGTGCTGGTCAGTGCCCTC | | | | |
| | GTGCTGATGGCCATGGTCA | | | | |
| | AGAGAAAGGATTCCAGAGG | | | | |
| | CTAG | | | | |
| | (SEQ ID NO: 323) | | | | |
| 14 | TGGGGGTCTTGGGTGCTTGC | MGPRLLFWALL | GAGGCTGGAG | EAGVTQSPTHLI | TCTG |
| | TTCCCCTTCCTTCTGCCTTGG | CLLGTGPVEAG | TCACACAAAGT | KTRGQQATLRC | GGCA |
| | ATAACAGCAGGAGGCCCTCA | VTQSPTHLIKTR | CCCACACACCT | SPISGHTSVYW | CACC |
| | TCAGTTATGGACCCTTGATCT | GQQATLRCSPIS | GATCAAAACG | YQQALGLGLQF | AGT |
| | TGGACTTCCCAGTCTCCAGA | GHTSVYWYQQ | AGAGGACAGC | LLWYDEGEERN | (SEQ |
| | TCTACAACCCAGCTCTGCTTT | ALGLGLQFLLW | AAGCGACTCTG | RGNFPPRFSGR | ID NO: |
| | GGATCTGATCAGATGGACTA | YDEGEERNRGN | AGATGCTCTCC | QFPNYSSELNV | 331) |
| | AATCTTGGGGACTCTGCACC | FPPRFSGRQFPN | TATCTCTGGGC | NALELEDSALY | |
| | ACTGGCCACTGAGGAAAGGG | YSSELNVNALE | ACACCAGTGTG | LCASSLQTSYE | |
| | AAGAGAATGTTGCCTGGGAC | LEDSALYLCAS | TACTGGTACCA | QYFGPGTRLTV | |
| | AGGAAAATATAGAAAATGA | SLQTSYEQYFG | ACAGGCCCTG | T | |
| | AGGCCCAGAACTCACTCGGC | PGTRLTVTEDL | GGTCTGGGCCT | (SEQ ID NO: | |
| | TCTTCCCCAGGAAGACCAAG | KNVFPPEVAVF | CCAGTTCCTCC | 620) | |
| | CCCTGAATCAGGTGCAGTGC | EPSEAEISHTQ | TTTGGTATGAC | | |
| | CGCCTGGCCCACTGTGCCAT | KATLVCLATG | GAGGGTGAAG | | |
| | GGGACCCAGGCTCCTCTTCT | FYPDHVELSW | AGAGAAACAG | | |
| | GGGCACTGCTTTGTCTCCTCG | WVNGKEVHSG | AGGAAACTTCC | | |
| | GAACAGGCCCAGTGGAGGCT | VSTDPQPLKEQ | CTCCTAGATTT | | |
| | GGAGTCACACAAAGTCCCAC | PALNDSRYCLS | TCAGGTCGCCA | | |
| | ACACCTGATCAAAACGAGAG | SRLRVSATFW | GTTCCCTAATT | | |
| | GACAGCAAGCGACTCTGAGA | QNPRNHFRCQ | ATAGCTCTGAG | | |
| | TGCTCTCCTATCTCTGGGCAC | VQFYGLSEND | CTGAATGTGAA | | |
| | ACCAGTGTGTACTGGTACCA | EWTQDRAKPV | CGCCTTGGAGC | | |
| | ACAGGCCCTGGGTCTGGGCC | TQIVSAEAWG | TGGAGGACTC | | |
| | TCCAGTTCCTCCTTTGGTATG | RADCGFTSESY | GGCCCTGTATC | | |
| | ACGAGGGTGAAGAGAGAAA | QQGVLSATILY | TCTGTGCCAGC | | |
| | CAGAGGAAACTTCCCTCCTA | EILLGKATLYA | AGCTTACAGAC | | |
| | GATTTTCAGGTCGCCAGTTC | VLVSALVLMA | ATCCTACGAGC | | |
| | CCTAATTATAGCTCTGAGCT | MVKRKDSRG | AGTACTTCGGG | | |
| | GAATGTGAACGCCTTGGAGC | (SEQ ID NO: | CCGGGCACCA | | |
| | TGGAGGACTCGGCCCTGTAT | 618) | GGCTCACGGTC | | |
| | CTCTGTGCCAGCAGCTTACA | | ACAG | | |
| | GACATCCTACGAGCAGTACT | | (SEQ ID NO: | | |
| | TCGGGCCGGGCACCAGGCTC | | 619) | | |
| | ACGGTCACAGAGGACCTGA | | | | |
| | AAAACGTGTTCCCACCCGA | | | | |
| | GGTCGCTGTGTTTGAGCCA | | | | |
| | TCAGAAGCAGAGATCTCCC | | | | |
| | ACACCCAAAAGGCCACACT | | | | |
| | GGTATGCCTGGCCACAGGC | | | | |
| | TTCTACCCCGACCACGTGG | | | | |
| | AGCTGAGCTGGTGGGTGAA | | | | |
| | TGGGAAGGAGGTGCACAG | | | | |
| | TGGGGTCAGCACAGACCCG | | | | |
| | CAGCCCCTCAAGGAGCAGC | | | | |
| | CCGCCCTCAATGACTCCAG | | | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCTGTC | | | | |
| | ACCCAGATCGTCAGCGCCG | | | | |
| | AGGCCTGGGGTAGAGCAG | | | | |
| | ACTGTGGCTTCACCTCCGA | | | | |
| | GTCTTACCAGCAAGGGGTC | | | | |
| | CTGTCTGCCACCATCCTCT | | | | |
| | ATGAGATCTTGCTAGGGAA | | | | |
| | GGCCACCTTGTATGCCGTG | | | | |
| | CTGGTCAGTGCCCTCGTGC | | | | |
| | TGATGGCCATGGTCAAGAG | | | | |
| | AAAGGATTCCAGAGGCTAG | | | | |
| | (SEQ ID NO: 330) | | | | |
| 15 | GGGCTCAGCCTGAGTTGGCT | MGPQLLGYVV | GAAGCCCAAG | EAQVTQNPRYL | ATGA |
| | GTGTTGCGTTTGTACACTTCA | LCLLGAGPLEA | TGACCCAGAA | ITVTGKKLTVT | ACCA |
| | GCCCTCTGAGCTGAAGTGGG | QVTQNPRYLIT | CCCAAGATACC | CSQNMNHEYM | TGAG |
| | AGTGGTTTCTCTCCTGAAAA | VTGKKLTVTCS | TCATCACAGTG | SWYRQDPGLGL | TAT |
| | TGTTTCTGAGGCCCAAATAG | QNMNHEYMSW | ACTGGAAAGA | RQIYYSMNVEV | SEQ |
| | CTGAAGAGGTGGAGACGTTA | YRQDPGLGLRQ | AGTTAACAGTG | TDKGDVPEGYK | ID NO: |
| | CAGAAACCACCTGGAGCCCC | IYYSMNVEVTD | ACTTGTTCTCA | VSRKEKRNFPLI | 338) |
| | CAGAACTGGCAGACACCTGC | KGDVPEGYKVS | GAATATGAAC | LESPSPNQTSLY | |
| | CTGATGCTGCCATGGGCCCC | RKEKRNFPLILE | CATGAGTATAT | FCASSLSWGTG | |
| | CAGCTCCTTGGCTATGTGGT | SPSPNQTSLYFC | GTCCTGGTATC | KRADTQYFGPG | |
| | CCTTTGCCTTCTAGGAGCAG | ASSLSWGTGKR | GACAAGACCC | TRLTVL | |
| | GCCCCCTGGAAGCCCAAGTG | ADTQYFGPGTR | AGGGCTGGGC | (SEQ ID NO: | |
| | ACCCAGAACCCAAGATACCT | LTVLEDLKNVF | TTAAGGCAGAT | 623) | |
| | CATCACAGTGACTGGAAAGA | PPEVAVFEPSE | CTACTATTCAA | | |
| | AGTTAACAGTGACTTGTTCT | AEISHTQKATL | TGAATGTTGAG | | |
| | CAGAATATGAACCATGAGTA | VCLATGFYPD | GTGACTGATAA | | |
| | TATGTCCTGGTATCGACAAG | HVELSWWVN | GGGAGATGTTC | | |
| | ACCCAGGGCTGGGCTTAAGG | GKEVHSGVST | CTGAAGGGTA | | |
| | CAGATCTACTATTCAATGAA | DPQPLKEQPA | CAAAGTCTCTC | | |
| | TGTTGAGGTGACTGATAAGG | LNDSRYCLSSR | GAAAAGAGAA | | |
| | GAGATGTTCCTGAAGGGTAC | LRVSATFWQN | GAGGAATTTCC | | |
| | AAAGTCTCTCGAAAAGAGAA | PRNHERCQVQ | CCCTGATCCTG | | |
| | GAGGAATTTCCCCCTGATCC | FYGLSENDEW | GAGTCGCCCA | | |
| | TGGAGTCGCCCAGCCCCAAC | TQDRAKPVTQI | GCCCCAACCA | | |
| | CAGACCTCTCTGTACTTCTGT | VSAEAWGRAD | GACCTCTCTGT | | |
| | GCCAGCAGTTTATCGTGGGG | CGFTSESYQQ | ACTTCTGTGCC | | |
| | GACCGGCAAAAGAGCAGAT | GVLSATILYEI | AGCAGTTTATC | | |
| | ACGCAGTATTTTGGCCCAGG | LLGKATLYAV | GTGGGGGACC | | |
| | CACCCGGCTGACAGTGCTCG | LVSALVLMAM | GGCAAAAGAG | | |
| | AGGACCTGAAAAACGTGTT | VKRKDSRG | CAGATACGCA | | |
| | CCCACCCGAGGTCGCTGTG | (SEQ ID NO: | GTATTTTGGCC | | |
| | TTTGAGCCATCAGAAGCAG | 621) | CAGGCACCCG | | |
| | AGATCTCCCACACCCAAAA | | GCTGACAGTGC | | |
| | GGCCACACTGGTGTGCCTG | | TCG | | |
| | GCCACAGGCTTCTACCCCG | | (SEQ ID NO: | | |
| | ACCACGTGGAGCTGAGCTG | | 622) | | |
| | GTGGGTGAATGGGAAGGA | | | | |
| | GGTGCACAGTGGGGTCAG | | | | |
| | CACAGACCCGCAGCCCCTC | | | | |
| | AAGGAGCAGCCCGCCCTCA | | | | |
| | ATGACTCCAGATACTGCCT | | | | |
| | GAGCAGCCGCCTGAGGGT | | | | |
| | CTCGGCCACCTTCTGGCAG | | | | |
| | AACCCCCGCAACCACTTCC | | | | |
| | GCTGTCAAGTCCAGTTCTA | | | | |
| | CGGGCTCTCGGAGAATGAC | | | | |
| | GAGTGGACCCAGGATAGG | | | | |
| | GCCAAACCTGTCACCCAGA | | | | |
| | TCGTCAGCGCCGAGGCCTG | | | | |
| | GGGTAGAGCAGACTGTGG | | | | |
| | CTTCACCTCCGAGTCTTAC | | | | |
| | CAGCAAGGGGTCCTGTCTG | | | | |
| | CCACCATCCTCTATGAGAT | | | | |
| | CTTGCTAGGGAAGGCCACC | | | | |
| | TTGTATGCCGTGCTGGTCA | | | | |
| | GTGCCCTCGTGCTGATGGC | | | | |
| | CATGGTCAAGAGAAAGGAT | | | | |
| | TCCAGAGGCTAG (SEQ ID | | | | |
| | NO: 337) | | | | |
| 16 | CGAAGGCGGAGGGTGGAAT | MGTSLLCWMA | GATACTGGAGT | DTGVSQDPRHK | TCTG |
| | GCGGGCAGCAGCCCCCTGGA | LCLLGADHADT | CTCCCAGGACC | ITKRGQNVTFR | AACA |
| | GGGCTGAGTGGGGAAAACA | GVSQDPRHKIT | CCAGACACAA | CDPISEHNRLY | CAAC |
| | AAATGGACCTCACAGAAGCT | KRGQNVTFRCD | GATCACAAAG | WYRQTLGQGP | CGC |
| | GTGTGTGTGGAAACCCACTT | PISEHNRLYWY | AGGGGACAGA | EFLTYFQNEAQ | (SEQ |
| | CTGACTTATCACTTGTCATGA | RQTLGQGPEFL | ATGTAACTTTC | LEKSRLLSDRFS | ID NO: |
| | ATTCTATGCTTCATGGTGTTA | TYFQNEAQLEK | AGGTGTGATCC | AERPKGSFSTLE | 345) |
| | CACCGTTTATTGTTTCTGATG | SRLLSDRFSAER | AATTTCTGAAC | IQRTEQGDSAM | |
| | AGTGACAGTAATTATTTTCTT | PKGSFSTLEIQR | ACAACCGCCTT | YLCASSLVGGG | |
| | TCTTGCTGGTACATAATAAA | TEQGDSAMYLC | TATTGGTACCG | SNEQFFGPGTR | |
| | GTGGTGCACATCAGAGTTGC | ASSLVGGGSNE | ACAGACCCTG | LTVL | |
| | TGCCATCTTAGACTTAACTC | QFFGPGTRLTV | GGGCAGGGCC | (SEQ ID NO: | |
| | ATCAGTATCAGGTGATCCTG | LEDLKNVFPPE | CAGAGTTTCTG | 626) | |
| | AGGCTCAGTGATGTCACTGT | VAVFEPSEAEI | ACTTACTTCCA | | |
| | GGGAACTGCTCTGTGGCGAC | SHTQKATLVC | GAATGAAGCT | | |
| | AAGGACGTCCCTCATCCTCT | LATGFYPDHV | CAACTAGAAA | | |
| | GCTCCTGCTCACAGTGACCC | ELSWWVNGKE | AATCAAGGCT | | |
| | TGATCTGGTAAAGCTCCCAT | VHSGVSTDPQP | GCTCAGTGATC | | |
| | CCTGCCCTGACCCTGCCATG | LKEQPALNDS | GGTTCTCTGCA | | |
| | GGCACCAGCCTCCTCTGCTG | RYCLSSRLRVS | GAGAGGCCTA | | |
| | GATGGCCCTGTGTCTCCTGG | ATFWQNPRNH | AGGGATCTTTC | | |
| | GGGCAGATCACGCAGATACT | FRCQVQFYGL | TCCACCTTGGA | | |
| | GGAGTCTCCCAGGACCCCAG | SENDEWTQDR | GATCCAGCGC | | |
| | ACACAAGATCACAAAGAGG | AKPVTQIVSAE | ACAGAGCAGG | | |
| | GGACAGAATGTAACTTTCAG | AWGRADCGFT | GGGACTCGGC | | |
| | GTGTGATCCAATTTCTGAAC | SESYQQGVLSA | CATGTATCTCT | | |
| | ACAACCGCCTTTATTGGTAC | TILYEILLGKA | GTGCCAGCAG | | |
| | CGACAGACCCTGGGGCAGGG | TLYAVLVSALV | CTTAGTTGGCG | | |
| | CCCAGAGTTTCTGACTTACTT | LMAMVKRKDS | GCGGGAGCAA | | |
| | CCAGAATGAAGCTCAACTAG | RG | TGAGCAGTTCT | | |
| | AAAAATCAAGGCTGCTCAGT | (SEQ ID NO: | TCGGGCCAGG | | |
| | GATCGGTTCTCTGCAGAGAG | 624) | GACACGGCTC | | |
| | GCCTAAGGGATCTTTCTCCA | | ACCGTGCTAG | | |
| | CCTTGGAGATCCAGCGCACA | | (SEQ ID NO: | | |
| | GAGCAGGGGGACTCGGCCAT | | 625) | | |
| | GTATCTCTGTGCCAGCAGCT | | | | |
| | TAGTTGGCGGCGGGAGCAAT | | | | |
| | GAGCAGTTCTTCGGGCCAGG | | | | |
| | GACACGGCTCACCGTGCTAG | | | | |
| | AGGACCTGAAAAACGTGTT | | | | |
| | CCCACCCGAGGTCGCTGTG | | | | |
| | TTTGAGCCATCAGAAGCAG | | | | |
| | AGATCTCCCACACCCAAAA | | | | |
| | GGCCACACTGGTGTGCCTG | | | | |
| | GCCACAGGCTTCTACCCCG | | | | |
| | ACCACGTGGAGCTGAGCTG | | | | |
| | GTGGGTGAATGGGAAGGA | | | | |
| | GGTGCACAGTGGGGTCAG | | | | |
| | CACAGACCCGCAGCCCCTC | | | | |
| | AAGGAGCAGCCCGCCCTCA | | | | |
| | ATGACTCCAGATACTGCCT | | | | |
| | GAGCAGCCGCCTGAGGGT | | | | |
| | CTCGGCCACCTTCTGGCAG | | | | |
| | AACCCCCGCAACCACTTCC | | | | |
| | GCTGTCAAGTCCAGTTCTA | | | | |
| | CGGGCTCTCGGAGAATGAC | | | | |
| | GAGTGGACCCAGGATAGG | | | | |
| | GCCAAACCTGTCACCCAGA | | | | |
| | TCGTCAGCGCCGAGGCCTG | | | | |
| | GGGTAGAGCAGACTGTGG | | | | |
| | CTTCACCTCCGAGTCTTAC | | | | |
| | CAGCAAGGGGTCCTGTCTG | | | | |
| | CCACCATCCTCTATGAGAT | | | | |
| | CTTGCTAGGGAAGGCCACC | | | | |
| | TTGTATGCCGTGCTGGTCA | | | | |
| | GTGCCCTCGTGCTGATGGC | | | | |
| | CATGGTCAAGAGAAAGGAT | | | | |
| | TCCAGAGGCTAG | | | | |
| | (SEQ ID NO: 344) | | | | |
| 17 | GACCTCCCACTCCTACCCAG | MGTSLLCWMA | GATACTGGAGT | DTGVSQDPRHK | TCTG |
| | ACCGTGGATGGGCAGGAAAT | LCLLGADHADT | CTCCCAGGACC | ITKRGQNVTFR | AACA |
| | GCAGGAACAGAGCCAGAAA | GVSQDPRHKIT | CCAGACACAA | CDPISEHNRLY | CAAC |
| | CAGGAGATCTCCAAGGAAGG | KRGQNVTFRCD | GATCACAAAG | WYRQTLGQGP | CGC |
| | TTGACAGTCAGCACTGGGAT | PISEHNRLYWY | AGGGGACAGA | EFLTYFQNEAQ | (SEQ |
| | CGTCTGTGTAAAGTGCTGCT | RQTLGQGPEFL | ATGTAACTTTC | LEKSRLLSDRFS | ID NO: |
| | GAAGCAGCCAGGTGGCATGT | TYFQNEAQLEK | AGGTGTGATCC | AERPKGSFSTLE | 352) |
| | CCAGCCGACAATGCGAAAGG | SRLLSDRFSAER | AATTTCTGAAC | IQRTEQGDSAM | |
| | AAAAAGTGAGAAGACTTCCC | PKGSFSTLEIQR | ACAACCGCCTT | YLCASSLVGGT | |
| | GAAGGCGGAGGGTGGAATG | TEQGDSAMYLC | TATTGGTACCG | EAFFGQGTRLT | |
| | CGGGCAGCAGCCCCCTGGAG | ASSLVGGTEAF | ACAGACCCTG | VV | |
| | GGCTGAGTGGGGAAAACAA | FGQGTRLTVVE | GGGCAGGGCC | (SEQ ID NO: | |
| | AATGGACCTCACAGAAGCTG | DLNKVFPPEVA | CAGAGTTTCTG | 629) | |
| | TGTGTGTGGAAACCCACTTC | VFEPSEAEISH | ACTTACTTCCA | | |
| | TGACTTATCACTTGTCATGA | TQKATLVCLA | GAATGAAGCT | | |
| | ATTCTATGCTTCATGGTGTTA | TGFYPDHVELS | CAACTAGAAA | | |
| | CACCGTTTATTGTTTCTGATG | WWVNGKEVH | AATCAAGGCT | | |
| | AGTGACAGTAATTATTTTCTT | SGVSTDPQPLK | GCTCAGTGATC | | |
| | TCTTGCTGGTACATAATAAA | EQPALNDSRY | GGTTCTCTGCA | | |
| | GTGGTGCACATCAGAGTTGC | CLSSRLRVSAT | GAGAGGCCTA | | |
| | TGCCATCTTAGACTTAACTC | FWQNPRNHFR | AGGGATCTTTC | | |
| | ATCAGTATCAGGTGATCCTG | CQVQFYGLSE | TCCACCTTGGA | | |
| | AGGCTCAGTGATGTCACTGT | NDEWTQDRAK | GATCCAGCGC | | |
| | GGGAACTGCTCTGTGGCGAC | PVTQIVSAEAW | ACAGAGCAGG | | |
| | AAGGACGTCCCTCATCCTCT | GRADCGFTSVS | GGGACTCGGC | | |
| | GCTCCTGCTCACAGTGACCC | YQQGVLSATIL | CATGTATCTCT | | |
| | TGATCTGGTAAAGCTCCCAT | YEILLGKATLY | GTGCCAGCAG | | |
| | CCTGCCCTGACCCTGCCATG | AVLVSALVLM | CTTAGTCGGAG | | |
| | GGCACCAGCCTCCTCTGCTG | AMVKRKDF | GCACTGAAGCT | | |
| | GATGGCCCTGTGTCTCCTGG | (SEQ ID NO: | TTCTTTGGACA | | |
| | GGGCAGATCACGCAGATACT | 627) | AGGCACCAGA | | |
| | GGAGTCTCCCAGGACCCCAG | | CTCACAGTTGT | | |
| | ACACAAGATCACAAAGAGG | | AG | | |
| | GGACAGAATGTAACTTTCAG | | (SEQ ID NO: | | |
| | GTGTGATCCAATTTCTGAAC | | 628) | | |
| | ACAACCGCCTTTATTGGTAC | | | | |
| | CGACAGACCCTGGGGCAGGG | | | | |
| | CCCAGAGTTTCTGACTTACTT | | | | |
| | CCAGAATGAAGCTCAACTAG | | | | |
| | AAAAATCAAGGCTGCTCAGT | | | | |
| | GATCGGTTCTCTGCAGAGAG | | | | |
| | GCCTAAGGGATCTTTCTCCA | | | | |
| | CCTTGGAGATCCAGCGCACA | | | | |
| | GAGCAGGGGGACTCGGCCAT | | | | |
| | GTATCTCTGTGCCAGCAGCT | | | | |
| | TAGTCGGAGGCACTGAAGCT | | | | |
| | TTCTTTGGACAAGGCACCAG | | | | |
| | ACTCACAGTTGTAGAGGACC | | | | |
| | TGAACAAGGTGTTCCCACC | | | | |
| | CGAGGTCGCTGTGTTTGAG | | | | |
| | CCATCAGAAGCAGAGATCT | | | | |
| | CCCACACCCAAAAGGCCAC | | | | |
| | ACTGGTGTGCCTGGCCACA | | | | |
| | GGCTTCTACCCCGACCACG | | | | |
| | TGGAGCTGAGCTGGTGGGT | | | | |
| | GAATGGGAAGGAGGTGCA | | | | |
| | CAGTGGGGTCAGCACAGAC | | | | |
| | CCGCAGCCCCTCAAGGAGC | | | | |
| | AGCCCGCCCTCAATGACTC | | | | |
| | CAGATACTGCCTGAGCAGC | | | | |
| | CGCCTGAGGGTCTCGGCCA | | | | |
| | CCTTCTGGCAGAACCCCCG | | | | |
| | CAACCACTTCCGCTGTCAA | | | | |
| | GTCCAGTTCTACGGGCTCT | | | | |
| | CGGAGAATGACGAGTGGA | | | | |
| | CCCAGGATAGGGCCAAACC | | | | |
| | CGTCACCCAGATCGTCAGC | | | | |
| | GCCGAGGCCTGGGGTAGA | | | | |
| | GCAGACTGTGGCTTTACCT | | | | |
| | CGGTGTCCTACCAGCAAGG | | | | |
| | GGTCCTGTCTGCCACCATC | | | | |
| | CTCTATGAGATCCTGCTAG | | | | |
| | GGAAGGCCACCCTGTATGC | | | | |
| | TGTGCTGGTCAGCGCCCTT | | | | |
| | GTGTTGATGGCCATGGTCA | | | | |
| | AGAGAAAGGATTTCTGA | | | | |
| | (SEQ ID NO: 351) | | | | |
| 18 | GGGCCATCTTAGACTTAACT | MGTSLLCWMA | GATACTGGAGT | DTGVSQDPRHK | TCTG |
| | CATCAGTATCAGGTGATCCT | LCLLGADHADT | CTCCCAGGACC | ITKRGQNVTFR | AACA |
| | GAGGCTCAGTGATGTCACTG | GVSQDPRHKIT | CCAGACACAA | CDPISEHNRLY | CAAC |
| | TGGGAACTGCTCTGTGGCGA | KRGQNVTFRCD | GATCACAAAG | WYRQTLGQGP | CGC |
| | CAAGGACGTCCCTCATCCTC | PISEHNRLYWY | AGGGGACAGA | EFLTYFQNEAQ | (SEQ |
| | TGCTCCTGCTCACAGTGACC | RQTLGQGPEFL | ATGTAACTTTC | LEKSRLLSDRFS | ID NO: |
| | CTGATCTGGTAAAGCTCCCA | TYFQNEAQLEK | AGGTGTGATCC | AERPKGSFSTLE | 359) |
| | TCCTGCCCTGACCCTGCCAT | SRLLSDRESAER | AATTTCTGAAC | IQRTEQGDSAM | |
| | GGGCACCAGCCTCCTCTGCT | PKGSFSTLEIQR | ACAACCGCCTT | YLCASSPDRNL | |
| | GGATGGCCCTGTGTCTCCTG | TEQGDSAMYLC | TATTGGTACCG | GQYFGPGTRLT | |
| | GGGGCAGATCACGCAGATAC | ASSPDRNLGQY | ACAGACCCTG | VT | |
| | TGGAGTCTCCCAGGACCCCA | FGPGTRLTVTE | GGGCAGGGCC | (SEQ ID NO: | |
| | GACACAAGATCACAAAGAG | DLKNVFPPEVA | CAGAGTTTCTG | 632) | |
| | GGGACAGAATGTAACTTTCA | VFEPSEAEISH | ACTTACTTCCA | | |
| | GGTGTGATCCAATTTCTGAA | TQKATLVCLA | GAATGAAGCT | | |
| | CACAACCGCCTTTATTGGTA | TGFYPDHVELS | CAACTAGAAA | | |
| | CCGACAGACCCTGGGGCAGG | WWVNGKEVH | AATCAAGGCT | | |
| | GCCCAGAGTTTCTGACTTAC | SGVSTDPQPLK | GCTCAGTGATC | | |
| | TTCCAGAATGAAGCTCAACT | EQPALNDSRY | GGTTCTCTGCA | | |
| | AGAAAAATCAAGGCTGCTCA | CLSSRLRVSAT | GAGAGGCCTA | | |
| | GTGATCGGTTCTCTGCAGAG | FWQNPRNHFR | AGGGATCTTTC | | |
| | AGGCCTAAGGGATCTTTCTC | CQVQFYGLSE | TCCACCTTGGA | | |
| | CACCTTGGAGATCCAGCGCA | NDEWTQDRAK | GATCCAGCGC | | |
| | CAGAGCAGGGGGACTCGGCC | PVTQIVSAEAW | ACAGAGCAGG | | |
| | ATGTATCTCTGTGCCAGCAG | GRADCGFTSES | GGGACTCGGC | | |
| | CCCCGATCGGAATCTCGGGC | YQQGVLSATIL | CATGTATCTCT | | |
| | AGTACTTCGGGCCGGGCACC | YEILLGKATLY | GTGCCAGCAG | | |
| | AGGCTCACGGTCACAGAGGA | AVLVSALVLM | CCCCGATCGGA | | |
| | CCTGAAAAACGTGTTCCCA | AMVKRKDSRG | ATCTCGGGCAG | | |
| | CCCGAGGTCGCTGTGTTTG | (SEQ ID NO: | TACTTCGGGCC | | |
| | AGCCATCAGAAGCAGAGAT | 630) | GGGCACCAGG | | |
| | CTCCCACACCCAAAAGGCC | | CTCACGGTCAC | | |
| | ACACTGGTGTGCCTGGCCA | | AG | | |
| | CAGGCTTCTACCCCGACCA | | (SEQ ID NO: | | |
| | CGTGGAGCTGAGCTGGTG | | 631) | | |
| | GGTGAATGGGAAGGAGGT | | | | |
| | GCCGTGGGGTCAGCACA | | | | |
| | GACCCAGCCCCTCAAGG | | | | |
| | AGCCCGCCCTCAATGA | | | | |
| | CTCCAGATACTGCCTGAGC | | | | |
| | AGCCCTGAGGGTCTCGG | | | | |
| | CCACCTTCTGGCAGAACCC | | | | |
| | CCGCACCACTTCCGCTGT | | | | |
| | CAAGTCCAGTTCTACGGGC | | | | |
| | TCTCGGAGAATGACGAGTG | | | | |
| | GACCCGGATAGGGCCAAA | | | | |
| | CCTGTCACCCAGATCGTCA | | | | |
| | GCGCCGAGGCCTGGGGTA | | | | |
| | GAGCAGACTGTGGCTTCAC | | | | |
| | CTCCGAGTCTTACCAGCAA | | | | |
| | GGGGTCCTGTCTGCCACCA | | | | |
| | TCCTCTATGAGATCTTGCT | | | | |
| | AGGGAAGGCCACCTTGTAT | | | | |
| | GCCGTGCTGGTCAGTGCCC | | | | |
| | TCGTGCTGATGGCCATGGT | | | | |
| | CAAGAGAAAGGATTCCAGA | | | | |
| | GGCTAG (SEQ ID NO: 358) | | | | |
| 19 | GGGACATTGGCTAATATGCT | MVSRLLSLVSL | GAAGCTGGAG | EAGVTQFPSHS | TCTG |
| | GATGTCACTGGAGGCCACAT | CLLGAKHIEAG | TTACTCAGTTC | VIEKGQTVTLR | GACA |
| | CTTACAGGGCCAAGAGACAG | VTQFPSHSVIEK | CCCAGCCACA | CDPISGHDNLY | TGAT |
| | ATTTGCTTTCCTTTTTCTCAT | GQTVTLRCDPIS | GCGTAATAGA | WYRRVMGKEI | AAT |
| | GCTTGTAAGCTCCTTCATCTG | GHDNLYWYRR | GAAGGGCCAG | KFLLHFVKESK | |
| | GAAATGTGATTTACCTGGGT | VMGKEIKFLLH | ACTGTGACTCT | QDESGMPNNRF | |
| | CCTGCCATGGTTTCCAGGCTT | FVKESKQDESG | GAGATGTGAC | LAERTGGTYST | (SEQ |
| | CTCAGTTTAGTGTCCCTTTGT | MPNNRFLAERT | CCAATTTCTGG | LKVQPAELEDS | ID NO: |
| | CTCCTGGGAGCAAAGCACAT | GGTYSTLKVQP | ACATGATAATC | GVYFCASSQVQ | 366) |
| | AGAAGCTGGAGTTACTCAGT | AELEDSGVYFC | TTTATTGGTAT | AFNEQFFGPGT | |
| | TCCCCAGCCACAGCGTAATA | ASSQVQAFNEQ | CGACGTGTTAT | RLTVL | |
| | GAGAAGGGCCAGACTGTGAC | FFGPGTRLTVL | GGGAAAAGAA | (SEQ ID NO: | |
| | TCTGAGATGTGACCCAATTT | EDLKNVFPPEV | ATAAAATTTCT | 635) | |
| | CTGGACATGATAATCTTTATT | AVFEPSEAEIS | GTTACATTTTG | | |
| | GGTATCGACGTGTTATGGGA | HTQKATLVCL | TGAAAGAGTCT | | |
| | AAAGAAATAAAATTTCTGTT | ATGFYPDHVE | AAACAGGATG | | |
| | ACATTTTGTGAAAGAGTCTA | LSWWVNGKE | AATCCGGTATG | | |
| | AACAGGATGAATCCGGTATG | VHSGVSTDPQP | CCCAACAATCG | | |
| | CCCAACAATCGATTCTTAGC | LKEQPALNDS | ATTCTTAGCTG | | |
| | TGAAAGGACTGGAGGGACGT | RYCLSSRLRVS | AAAGGACTGG | | |
| | ATTCTACTCTGAAGGTGCAG | ATFWQNPRNH | AGGGACGTATT | | |
| | CCTGCAGAACTGGAGGATTC | FRCQVQFYGL | CTACTCTGAAG | | |
| | TGGAGTTTATTTCTGTGCCAG | SENDEWTQDR | GTGCAGCCTGC | | |
| | CAGCCAAGTCCAGGCTTTTA | AKPVTQIVSAE | AGAACTGGAG | | |
| | ATGAGCAGTTCTTCGGGCCA | AWGRADCGFT | GATTCTGGAGT | | |
| | GGGACACGGCTCACCGTGCT | SESYQQGVLSA | TTATTTCTGTG | | |
| | AGAGGACCTGAAAAACGTG | TILYEILLGKA | CCAGCAGCCA | | |
| | TTCCCACCCGAGGTCGCTG | TLYAVLVSALV | AGTCCAGGCTT | | |
| | TGTTTGAGCCATCAGAAGC | LMAMVKRKDS | TTAATGAGCAG | | |
| | AGAGATCTCCCACACCCAA | RG | TTCTTCGGGCC | | |
| | AAGGCCACACTGGTGTGCC | (SEQ ID NO: | AGGGACACGG | | |
| | TGGCCACAGGCTTCTACCC | 633) | CTCACCGTGCT | | |
| | CGACCACGTGGAGCTGAGC | | AG | | |
| | TGGTGGGTGAATGGGAAG | | (SEQ ID NO: | | |
| | GAGGTGCACAGTGGGGTC | | 634) | | |
| | AGCACAGACCCGCAGCCCC | | | | |
| | TCAAGGAGCAGCCCGCCCT | | | | |
| | CAATGACTCCAGATACTGC | | | | |
| | CTGAGCAGCCGCCTGAGG | | | | |
| | GTCTCGGCCACCTTCTGGC | | | | |
| | AGAACCCCCGCAACCACTT | | | | |
| | CCGCTGTCAAGTCCAGTTC | | | | |
| | TACGGGCTCTCGGAGAATG | | | | |
| | ACGAGTGGACCCAGGATAG | | | | |
| | GGCCAAACCTGTCACCCAG | | | | |
| | ATCGTCAGCGCCGAGGCCT | | | | |
| | GGGGTAGAGCAGACTGTG | | | | |
| | GCTTCACCTCCGAGTCTTA | | | | |
| | CCAGCAAGGGGTCCTGTCT | | | | |
| | GCCACCATCCTCTATGAGA | | | | |
| | TCTTGCTAGGGAAGGCCAC | | | | |
| | CTTGTATGCCGTGCTGGTC | | | | |
| | AGTGCCCTCGTGCTGATGG | | | | |
| | CCATGGTCAAGAGAAAGGA | | | | |
| | TTCCAGAGGCTAG (SEQ ID | | | | |
| | NO: 365) | | | | |
| 20 | TCTCTTTTATGTTGCCATCCA | MRGQGRPSSSA | GAAGCTGGAG | EAGVAQSPRYK | TCTG |
| | AGTAAGACATGACTTGCCCC | FAHSDPDWAKL | TTGCCCAGTCT | IIEKRQSVAFWC | GCCA |
| | TCCTTGCCTTCTGCCATGATT | PSFPDPAMGTR | CCCAGATATAA | NPISGHATLYW | TGCT |
| | GTGAGGCCTCCCCAGCCATG | LLCWAALCLLG | GATTATAGAG | YQQILGQGPKL | ACC |
| | TGGAACTAGGAAAAGTGAAT | AELTEAGVAQS | AAAAGGCAGA | LIQFQNNGVVD | (SEQ |
| | CAAAACCAAGGGACATGCTG | PRYKIIEKRQSV | GTGTGGCTTTT | DSQLPKDRFSA | ID NO: |
| | AGACAACTGGAGAAATTTGA | AFWCNPISGHA | TGGTGCAATCC | ERLKGVDSTLK | 373) |
| | ATGTGAAGCATTTGTGGAGG | TLYWYQQILGQ | TATATCTGGCC | IQPAKLEDSAV | |
| | CAATGATGTCACTGTGGGAA | GPKLLIQFQNN | ATGCTACCCTT | YLCASSRGEPG | |
| | CTGCCATGAGAGGACAGGGA | GVVDDSQLPKD | TACTGGTACCA | SGANVLTFGAG | |
| | CGTCCCTCCTCCTCTGCTTTT | RFSAERLKGVD | GCAGATCCTGG | SRLTVL | |
| | GCTCACAGTGACCCTGATTG | STLKIQPAKLED | GACAGGGCCC | (SEQ ID NO: | |
| | GGCAAAGCTCCCATCCTTCC | SAVYLCASSRG | AAAGCTTCTGA | 638) | |
| | CTGACCCTGCCATGGGCACC | EPGSGANVLTF | TTCAGTTTCAG | | |
| | AGGCTCCTCTGCTGGGCGGC | GAGSRLTVLED | AATAACGGTGT | | |
| | CCTCTGTCTCCTGGGAGCAG | LKNVFPPEVAV | AGTGGATGATT | | |
| | AACTCACAGAAGCTGGAGTT | FEPSEAEISHT | CACAGTTGCCT | | |
| | GCCCAGTCTCCCAGATATAA | QKATLVCLAT | AAGGATCGATT | | |
| | GATTATAGAGAAAAGGCAG | GFYPDHVELS | TTCTGCAGAGA | | |
| | AGTGTGGCTTTTTGGTGCAA | WWVNGKEVH | GGCTCAAAGG | | |
| | TCCTATATCTGGCCATGCTAC | SGVSTDPQPLK | AGTAGACTCCA | | |
| | CCTTTACTGGTACCAGCAGA | EQPALNDSRY | CTCTCAAGATC | | |
| | TCCTGGGACAGGGCCCAAAG | CLSSRLRVSAT | CAGCCTGCAA | | |
| | CTTCTGATTCAGTTTCAGAAT | FWQNPRNHFR | AGCTTGAGGA | | |
| | AACGGTGTAGTGGATGATTC | CQVQFYGLSE | CTCGGCCGTGT | | |
| | ACAGTTGCCTAAGGATCGAT | NDEWTQDRAK | ATCTCTGTGCC | | |
| | TTTCTGCAGAGAGGCTCAAA | PVTQIVSAEAW | AGCAGTCGAG | | |
| | GGAGTAGACTCCACTCTCAA | GRADCGFTSES | GGGAGCCAGG | | |
| | GATCCAGCCTGCAAAGCTTG | YQQGVLSATIL | CTCTGGGGCCA | | |
| | AGGACTCGGCCGTGTATCTC | YEILLGKATLY | ACGTCCTGACT | | |
| | TGTGCCAGCAGTCGAGGGGA | AVLVSALVLM | TTCGGGGCCGG | | |
| | GCCAGGCTCTGGGGCCAACG | AMVKRKDSRG | CAGCAGGCTG | | |
| | TCCTGACTTTCGGGGCCGGC | (SEQ ID NO: | ACCGTGCTGG | | |
| | AGCAGGCTGACCGTGCTGGA | 636) | (SEQ ID NO: | | |
| | GGACCTGAAAAACGTGTTC | | 637) | | |
| | CCACCCGAGGTCGCTGTGT | | | | |
| | TTGAGCCATCAGAAGCAGA | | | | |
| | GATCTCCCACACCCAAAAG | | | | |
| | GCCACACTGGTGTGCCTGG | | | | |
| | CCACAGGCTTCTACCCCGA | | | | |
| | CCACGTGGAGCTGAGCTGG | | | | |
| | TGGGTGAATGGGAAGGAG | | | | |
| | GTGCACAGTGGGGTCAGCA | | | | |
| | CAGACCCGCAGCCCCTCAA | | | | |
| | GGAGCAGCCCGCCCTCAAT | | | | |
| | GACTCCAGATACTGCCTGA | | | | |
| | GCAGCCGCCTGAGGGTCTC | | | | |
| | GGCCACCTTCTGGCAGAAC | | | | |
| | CCCCGCAACCACTTCCGCT | | | | |
| | GTCAAGTCCAGTTCTACGG | | | | |
| | GCTCTCGGAGAATGACGAG | | | | |
| | TGGACCCAGGATAGGGCCA | | | | |
| | AACCTGTCACCCAGATCGT | | | | |
| | CAGCGCCGAGGCCTGGGG | | | | |
| | TAGAGCAGACTGTGGCTTC | | | | |
| | ACCTCCGAGTCTTACCAGC | | | | |
| | AAGGGGTCCTGTCTGCCAC | | | | |
| | CATCCTCTATGAGATCTTG | | | | |
| | CTAGGGAAGGCCACCTTGT | | | | |
| | ATGCCGTGCTGGTCAGTGC | | | | |
| | CCTCGTGCTGATGGCCATG | | | | |
| | GTCAAGAGAAAGGATTCCA | | | | |
| | GAGGCTAG (SEQ ID NO: 372) | | | | |
| 21 | GGGGCAGTGTAGGCAGAGG | MQCFLSLCAM | GACGCTGGAG | DAGVTQSPTHL | TCTG |
| | AGGAACTGTATCACCACAGA | GPGLLCWALLC | TCACCCAAAGT | IKTRGQQVTLR | GGCA |
| | AACTTCTGCCTTCACACATCC | LLGAGLVDAG | CCCACACACCT | CSPKSGHDTVS | TGAC |
| | CTCCAGCTAGGCAGGACAGG | VTQSPTHLIKTR | GATCAAAACG | WYQQALGQGP | ACT |
| | TAGAGAGTCCAGTGTCCTGG | GQQVTLRCSPK | AGAGGACAGC | QFIFQYYEEEER | (SEQ |
| | AGCACTAGACCTAAGGAAGG | SGHDTVSWYQ | AAGTGACTCTG | QRGNFPDRFSG | ID NO: |
| | CTGCATGGGGAGGACAAAG | QALGQGPQFIF | AGATGCTCTCC | HQFPNYSSELN | 380) |
| | GACAGTGACATCACAGGATA | QYYEEEERQRG | TAAGTCTGGGC | VNALLLGDSAL | |
| | CCCCTCCCATCAGGAAAATC | NFPDRFSGHQF | ATGACACTGTG | YLCASSSGTDPS | |
| | AAGGCCCAGAACTCACTCGG | PNYSSELNVNA | TCCTGGTACCA | GANVLTFGAGS | |
| | CTCTTCCCCAGGAGAACCAA | LLLGDSALYLC | ACAGGCCCTG | RLTVL | |
| | GCCCTGAATCAGATGCAGTG | ASSSGTDPSGA | GGTCAGGGGC | (SEQ ID NO: | |
| | CTTCCTGTCCCTCTGTGCCAT | NVLTFGAGSRL | CCCAGTTTATC | 641) | |
| | GGGCCCCGGGCTCCTCTGCT | TVLEDLKNVFP | TTTCAGTATTA | | |
| | GGGCACTGCTTTGTCTCCTG | PEVAVFEPSEA | TGAGGAGGAA | | |
| | GGAGCAGGCTTAGTGGACGC | EISHTQKATLV | GAGAGACAGA | | |
| | TGGAGTCACCCAAAGTCCCA | CLATGFFPDH | GAGGCAACTTC | | |
| | CACACCTGATCAAAACGAGA | VELSWWVNG | CCTGATCGATT | | |
| | GGACAGCAAGTGACTCTGAG | KEVHSGVSTDP | CTCAGGTCACC | | |
| | ATGCTCTCCTAAGTCTGGGC | QPLKEQPALN | AGTTCCCTAAC | | |
| | ATGACACTGTGTCCTGGTAC | DSRYCLSSRLR | TATAGCTCTGA | | |
| | CAACAGGCCCTGGGTCAGGG | VSATFWQNPR | GCTGAATGTGA | | |
| | GCCCCAGTTTATCTTTCAGTA | NHFRCQVQFY | ACGCCTTGTTG | | |
| | TTATGAGGAGGAAGAGAGA | GLSENDEWTQ | CTGGGGGACTC | | |
| | CAGAGAGGCAACTTCCCTGA | DRAKPVTQIVS | GGCCCTCTATC | | |
| | TCGATTCTCAGGTCACCAGT | AEAWGRADCG | TCTGTGCCAGC | | |
| | TCCCTAACTATAGCTCTGAG | FTSESYQQGVL | AGCTCCGGGA | | |
| | CTGAATGTGAACGCCTTGTT | SATILYEILLG | CAGACCCCTCT | | |
| | GCTGGGGGACTCGGCCCTCT | KATLYAVLVS | GGGGCCAACG | | |
| | ATCTCTGTGCCAGCAGCTCC | ALVLMAMVK | TCCTGACTTTC | | |
| | GGGACAGACCCCTCTGGGGC | RKDSRG | GGGGCCGGCA | | |
| | CAACGTCCTGACTTTCGGGG | (SEQ ID NO: | GCAGGCTGAC | | |
| | CCGGCAGCAGGCTGACCGTG | 639) | CGTGCTGG | | |
| | CTGGAGGACCTGAAAAACG | | (SEQ ID NO: | | |
| | TGTTCCCACCCGAGGTCGC | | 640) | | |
| | TGTGTTTGAGCCATCAGAA | | | | |
| | GCAGAGATCTCCCACACCC | | | | |
| | AAAAGGCCACACTGGTGTG | | | | |
| | CCTGGCCACAGGCTTCTTC | | | | |
| | CCTGACCACGTGGAGCTGA | | | | |
| | GCTGGTGGGTGAATGGGA | | | | |
| | AGGAGGTGCACAGTGGGG | | | | |
| | TCAGCACAGACCCGCAGCC | | | | |
| | CCTCAAGGAGCAGCCCGCC | | | | |
| | CTCAATGACTCCAGATACT | | | | |
| | GCCTGAGCAGCCGCCTGAG | | | | |
| | GGTCTCGGCCACCTTCTGG | | | | |
| | CAGAACCCCCGCAACCACT | | | | |
| | TCCGCTGTCAAGTCCAGTT | | | | |
| | CTACGGGCTCTCGGAGAAT | | | | |
| | GACGAGTGGACCCAGGATA | | | | |
| | GGGCCAAACCTGTCACCCA | | | | |
| | GATCGTCAGCGCCGAGGCC | | | | |
| | TGGGGTAGAGCAGACTGTG | | | | |
| | GCTTCACCTCCGAGTCTTA | | | | |
| | CCAGCAAGGGGTCCTGTCT | | | | |
| | GCCACCATCCTCTATGAGA | | | | |
| | TCTTGCTAGGGAAGGCCAC | | | | |
| | CTTGTATGCCGTGCTGGTC | | | | |
| | AGTGCCCTCGTGCTGATGG | | | | |
| | CCATGGTCAAGAGAAAGGA | | | | |
| | TTCCAGAGGCTAG (SEQ ID | | | | |
| | NO: 379) | | | | |
| 22 | GGGGTTTGTACACTTCAGCC | MGPQLLGYVV | GAAGCCCAAG | EAQVTQNPRYL | ATGA |
| | CTCTGAGCTGAAGTGGGAGT | LCLLGAGPLEA | TGACCCAGAA | ITVTGKKLTVT | ACCA |
| | GGTTTCTCTCCTGAAAATGTT | QVTQNPRYLIT | CCCAAGATACC | CSQNMNHEYM | TGAG |
| | TCTGAGGCCCAAATAGCTGA | VTGKKLTVTCS | TCATCACAGTG | SWYRQDPGLGL | TAT |
| | AGAGGTGGAGACGTTACAGA | QNMNHEYMSW | ACTGGAAAGA | RQIYYSMNVEV | (SEQ |
| | AACCACCTGGAGCCCCCAGA | YRQDPGLGLRQ | AGTTAACAGTG | TDKGDVPEGYK | ID NO: |
| | ACTGGCAGACACCTGCCTGA | IYYSMNVEVTD | ACTTGTTCTCA | VSRKEKRNFPLI | 387) |
| | TGCTGCCATGGGCCCCCAGC | KGDVPEGYKVS | GAATATGAAC | LESPSPNQTSLY | |
| | TCCTTGGCTATGTGGTCCTTT | RKEKRNFPLILE | CATGAGTATAT | FCASSTGQNIG | |
| | GCCTTCTAGGAGCAGGCCCC | SPSPNQTSLYFC | GTCCTGGTATC | GELFFGEGSRLT | |
| | CTGGAAGCCCAAGTGACCCA | ASSTGQNIGGE | GACAAGACCC | VL | |
| | GAACCCAAGATACCTCATCA | LFFGEGSRLTVL | AGGGCTGGGC | (SEQ ID NO: | |
| | CAGTGACTGGAAAGAAGTTA | EDLKNVFPPEV | TTAAGGCAGAT | 644) | |
| | ACAGTGACTTGTTCTCAGAA | AVFEPSEAEIS | CTACTATTCAA | | |
| | TATGAACCATGAGTATATGT | HTQKATLVCL | TGAATGTTGAG | | |
| | CCTGGTATCGACAAGACCCA | ATGFYPDHVE | GTGACTGATAA | | |
| | GGGCTGGGCTTAAGGCAGAT | LSWWVNGKE | GGGAGATGTTC | | |
| | CTACTATTCAATGAATGTTG | VHSGVSTDPQP | CTGAAGGGTA | | |
| | AGGTGACTGATAAGGGAGAT | LKEQPALNDS | CAAAGTCTCTC | | |
| | GTTCCTGAAGGGTACAAAGT | RYCLSSRLRVS | GAAAAGAGAA | | |
| | CTCTCGAAAAGAGAAGAGG | ATFWQNPRNH | GAGGAATTTCC | | |
| | AATTTCCCCCTGATCCTGGA | FRCQVQFYGL | CCCTGATCCTG | | |
| | GTCGCCCAGCCCCAACCAGA | SENDEWTQDR | GAGTCGCCCA | | |
| | CCTCTCTGTACTTCTGTGCCA | AKPVTQIVSAE | GCCCCAACCA | | |
| | GCAGTACCGGACAGAATATA | AWGRADCGFT | GACCTCTCTGT | | |
| | GGCGGGGAGCTGTTTTTTGG | SESYQQGVLSA | ACTTCTGTGCC | | |
| | AGAAGGCTCTAGGCTGACCG | TILYEILLGKA | AGCAGTACCG | | |
| | TACTGGAGGACCTGAAAAA | TLYAVLVSALV | GACAGAATAT | | |
| | CGTGTTCCCACCCGAGGTC | LMAMVKRKDS | AGGCGGGGAG | | |
| | GCTGTGTTTGAGCCATCAG | RG | CTGTTTTTTGG | | |
| | AAGCAGAGATCTCCCACAC | (SEQ ID NO: | AGAAGGCTCT | | |
| | CCAAAAGGCCACACTGGTG | 642) | AGGCTGACCGT | | |
| | TGCCTGGCCACAGGCTTCT | | ACTGG | | |
| | ACCCCGACCACGTGGAGCT | | (SEQ ID NO: | | |
| | GAGCTGGTGGGTGAATGG | | 643) | | |
| | GAAGGAGGTGCACAGTGG | | | | |
| | GGTCAGCACAGACCCGCAG | | | | |
| | CCCCTCAAGGAGCAGCCCG | | | | |
| | CCCTCAATGACTCCAGATA | | | | |
| | CTGCCTGAGCAGCCGCCTG | | | | |
| | AGGGTCTCGGCCACCTTCT | | | | |
| | GGCAGAACCCCCGCAACCA | | | | |
| | CTTCCGCTGTCAAGTCCAG | | | | |
| | TTCTACGGGCTCTCGGAGA | | | | |
| | ATGACGAGTGGACCCAGGA | | | | |
| | TAGGGCCAAACCTGTCACC | | | | |
| | CAGATCGTCAGCGCCGAGG | | | | |
| | CCTGGGGTAGAGCAGACTG | | | | |
| | TGGCTTCACCTCCGAGTCT | | | | |
| | TACCAGCAAGGGGTCCTGT | | | | |
| | CTGCCACCATCCTCTATGA | | | | |
| | GATCTTGCTAGGGAAGGCC | | | | |
| | ACCTTGTATGCCGTGCTGG | | | | |
| | TCAGTGCCCTCGTGCTGAT | | | | |
| | GGCCATGGTCAAGAGAAAG | | | | |
| | GATTCCAGAGGCTAG (SEQ | | | | |
| | ID NO: 386) | | | | |
| 23 | TGGGAGAAACAGGAGATCTC | MVHIRVAAILD | GATACTGGAGT | DTGVSQDPRHK | TCTG |
| | CAAGGAAGGTTGACAGTCAG | LTHQYQVILRL | CTCCCAGGACC | ITKRGQNVTFR | AACA |
| | CACTGGGATCGTCTGTGTAA | SDVTVGTALW | CCAGACACAA | CDPISEHNRLY | CAAC |
| | AGTGCTGCTGAAGCAGCCAG | RQGRPSSSAPA | GATCACAAAG | WYRQTLGQGP | CGC |
| | GTGGCATGTCCAGCCGACAA | HSDPDLVKLPS | AGGGGACAGA | EFLTYFQNEAQ | (SEQ |
| | TGCGAAAGGAAAAATGGTGC | CPDPAMGTSLL | ATGTAACTTTC | LEKSRLLSDRFS | ID NO: |
| | ACATCAGAGTTGCTGCCATC | CWMALCLLGA | AGGTGTGATCC | AERPKGSFSTLE | 394) |
| | TTAGACTTAACTCATCAGTA | DHADTGVSQDP | AATTTCTGAAC | IQRTEQGDSAM | |
| | TCAGGTGATCCTGAGGCTCA | RHKITKRGQNV | ACAACCGCCTT | YLCASSTHEKT | |
| | GTGATGTCACTGTGGGAACT | TFRCDPISEHNR | TATTGGTACCG | GWKSPLHFGNG | |
| | GCTCTGTGGCGACAAGGACG | LYWYRQTLGQ | ACAGACCCTG | TRLTVT | |
| | TCCCTCATCCTCTGCTCCTGC | GPEFLTYFQNE | GGGCAGGGCC | (SEQ ID NO: | |
| | TCACAGTGACCCTGATCTGG | AQLEKSRLLSD | CAGAGTTTCTG | 647) | |
| | TAAAGCTCCCATCCTGCCCT | RFSAERPKGSFS | ACTTACTTCCA | | |
| | GACCCTGCCATGGGCACCAG | TLEIQRTEQGDS | GAATGAAGCT | | |
| | CCTCCTCTGCTGGATGGCCCT | AMYLCASSTHE | CAACTAGAAA | | |
| | GTGTCTCCTGGGGGCAGATC | KTGWKSPLHFG | AATCAAGGCT | | |
| | ACGCAGATACTGGAGTCTCC | NGTRLTVTEDL | GCTCAGTGATC | | |
| | CAGGACCCCAGACACAAGAT | NKVFPPEVAVF | GGTTCTCTGCA | | |
| | CACAAAGAGGGGACAGAAT | EPSEAEISHTQ | GAGAGGCCTA | | |
| | GTAACTTTCAGGTGTGATCC | KATLVCLATG | AGGGATCTTTC | | |
| | AATTTCTGAACACAACCGCC | FYPDHVELSW | TCCACCTTGGA | | |
| | TTTATTGGTACCGACAGACC | WVNGKEVHSG | GATCCAGCGC | | |
| | CTGGGGCAGGGCCCAGAGTT | VSTDPQPLKEQ | ACAGAGCAGG | | |
| | TCTGACTTACTTCCAGAATG | PALNDSRYCLS | GGGACTCGGC | | |
| | AAGCTCAACTAGAAAAATCA | SRLRVSATFW | CATGTATCTCT | | |
| | AGGCTGCTCAGTGATCGGTT | QNPRNHFRCQ | GTGCCAGCAG | | |
| | CTCTGCAGAGAGGCCTAAGG | VQFYGLSEND | CACACATGAA | | |
| | GATCTTTCTCCACCTTGGAG | EWTQDRAKPV | AAGACAGGGT | | |
| | ATCCAGCGCACAGAGCAGGG | TQIVSAEAWG | GGAAATCACC | | |
| | GGACTCGGCCATGTATCTCT | RADCGFTSVSY | CCTCCACTTTG | | |
| | GTGCCAGCAGCACACATGAA | QQGVLSATILY | GGAATGGGAC | | |
| | AAGACAGGGTGGAAATCACC | EILLGKATLYA | CAGGCTCACTG | | |
| | CCTCCACTTTGGGAATGGGA | VLVSALVLMA | TGACAG | | |
| | CCAGGCTCACTGTGACAGAG | MVKRKDF | (SEQ ID NO: | | |
| | GACCTGAACAAGGTGTTCC | (SEQ ID NO: | 646) | | |
| | CACCCGAGGTCGCTGTGTT | 645) | | | |
| | TGAGCCATCAGAAGCAGAG | | | | |
| | ATCTCCCACACCCAAAAGG | | | | |
| | CCACACTGGTGTGCCTGGC | | | | |
| | CACAGGCTTCTACCCCGAC | | | | |
| | CACGTGGAGCTGAGCTGGT | | | | |
| | GGGTGAATGGGAAGGAGG | | | | |
| | TGCACAGTGGGGTCAGCAC | | | | |
| | AGACCCGCAGCCCCTCAAG | | | | |
| | GAGCAGCCCGCCCTCAATG | | | | |
| | ACTCCAGATACTGCCTGAG | | | | |
| | CAGCCGCCTGAGGGTCTCG | | | | |
| | GCCACCTTCTGGCAGAACC | | | | |
| | CCCGCAACCACTTCCGCTG | | | | |
| | TCAAGTCCAGTTCTACGGG | | | | |
| | CTCTCGGAGAATGACGAGT | | | | |
| | GGACCCAGGATAGGGCCA | | | | |
| | AACCCGTCACCCAGATCGT | | | | |
| | CAGCGCCGAGGCCTGGGG | | | | |
| | TAGAGCAGACTGTGGCTTT | | | | |
| | ACCTCGGTGTCCTACCAGC | | | | |
| | AAGGGGTCCTGTCTGCCAC | | | | |
| | CATCCTCTATGAGATCCTG | | | | |
| | CTAGGGAAGGCCACCCTGT | | | | |
| | ATGCTGTGCTGGTCAGCGC | | | | |
| | CCTTGTGTTGATGGCCATG | | | | |
| | GTCAAGAGAAAGGATTTCT | | | | |
| | GA (SEQ ID NO: 393) | | | | |
| 24 | GGGGCCAGACCTTGCCTGTG | MDTWLVCWAI | GAACCTGAAG | EPEVTQTPSHQ | TCTA |
| | GGGCCATGGGAGCTCAAAAT | FSLLKAGLTEPE | TCACCCAGACT | VTQMGQEVILR | ATCA |
| | GCCCCTCCTTTCCTCCACAGG | VTQTPSHQVTQ | CCCAGCCATCA | CVPISNHLYFY | CTTAT |
| | ACCAGATGCCTGAGCTAGGA | MGQEVILRCVPI | GGTCACACAG | WYRQILGQKVE | AC |
| | AAGGCCTCATTCCTGCTGTG | SNHLYFYWYR | ATGGGACAGG | FLVSFYNNEISE | (SEQ |
| | ATCCTGCCATGGATACCTGG | QILGQKVEFLV | AAGTGATCTTG | KSEIFDDQFSVE | ID NO: |
| | CTCGTATGCTGGGCAATTTTT | SFYNNEISEKSE | CGCTGTGTCCC | RPDGSNFTLKIR | 401) |
| | AGTCTCTTGAAAGCAGGACT | IFDDQFSVERPD | CATCTCTAATC | STKLEDSAMYF | |
| | CACAGAACCTGAAGTCACCC | GSNFTLKIRSTK | ACTTATACTTC | CASSTHSDRNL | |
| | AGACTCCCAGCCATCAGGTC | LEDSAMYFCAS | TATTGGTACAG | NTEAFFGQGTR | |
| | ACACAGATGGGACAGGAAG | STHSDRNLNTE | ACAAATCTTGG | LTVV | |
| | TGATCTTGCGCTGTGTCCCCA | AFFGQGTRLTV | GGCAGAAAGT | (SEQ ID NO: | |
| | TCTCTAATCACTTATACTTCT | VEDLNKVFPPE | CGAGTTTCTGG | 650) | |
| | ATTGGTACAGACAAATCTTG | VAVFEPSEAEI | TTTCCTTTTAT | | |
| | GGGCAGAAAGTCGAGTTTCT | SHTQKATLVC | AATAATGAAA | | |
| | GGTTTCCTTTTATAATAATGA | LATGFFPDHVE | TCTCAGAGAA | | |
| | AATCTCAGAGAAGTCTGAAA | LSWWVNGKE | GTCTGAAATAT | | |
| | TATTCGATGATCAATTCTCA | VHSGVSTDPQP | TCGATGATCAA | | |
| | GTTGAAAGGCCTGATGGATC | LKEQPALNDS | TTCTCAGTTGA | | |
| | AAATTTCACTCTGAAGATCC | RYCLSSRLRVS | AAGGCCTGAT | | |
| | GGTCCACAAAGCTGGAGGAC | ATFWQNPRNH | GGATCAAATTT | | |
| | TCAGCCATGTACTTCTGTGCC | FRCQVQFYGL | CACTCTGAAGA | | |
| | AGCAGTACGCACTCTGACAG | SENDEWTQDR | TCCGGTCCACA | | |
| | GAACTTGAACACTGAAGCTT | AKPVTQIVSAE | AAGCTGGAGG | | |
| | TCTTTGGACAAGGCACCAGA | AWGRADCGFT | ACTCAGCCATG | | |
| | CTCACAGTTGTAGAGGACCT | SVSYQQGVLSA | TACTTCTGTGC | | |
| | GAACAAGGTGTTCCCACCC | TILYEILLGKA | CAGCAGTACG | | |
| | GAGGTCGCTGTGTTTGAGC | TLYAVLVSALV | CACTCTGACAG | | |
| | CATCAGAAGCAGAGATCTC | LMAMVKRKD | GAACTTGAAC | | |
| | CCACACCCAAAAGGCCACA | F | ACTGAAGCTTT | | |
| | CTGGTGTGCCTGGCCACAG | (SEQ ID NO: | CTTTGGACAAG | | |
| | GCTTCTTCCCTGACCACGT | 648) | GCACCAGACTC | | |
| | GGAGCTGAGCTGGTGGGT | | ACAGTTGTAG | | |
| | GAATGGGAAGGAGGTGCA | | (SEQ ID NO: | | |
| | CAGTGGGGTCAGCACAGAC | | 649) | | |
| | CCGCAGCCCCTCAAGGAGC | | | | |
| | AGCCCGCCCTCAATGACTC | | | | |
| | CAGATACTGCCTGAGCAGC | | | | |
| | CGCCTGAGGGTCTCGGCCA | | | | |
| | CCTTCTGGCAGAACCCCCG | | | | |
| | CAACCACTTCCGCTGTCAA | | | | |
| | GTCCAGTTCTACGGGCTCT | | | | |
| | CGGAGAATGACGAGTGGA | | | | |
| | CCCAGGATAGGGCCAAACC | | | | |
| | CGTCACCCAGATCGTCAGC | | | | |
| | GCCGAGGCCTGGGGTAGA | | | | |
| | GCAGACTGTGGCTTTACCT | | | | |
| | CGGTGTCCTACCAGCAAGG | | | | |
| | GGTCCTGTCTGCCACCATC | | | | |
| | CTCTATGAGATCCTGCTAG | | | | |
| | GGAAGGCCACCCTGTATGC | | | | |
| | TGTGCTGGTCAGCGCCCTT | | | | |
| | GTGTTGATGGCCATGGTCA | | | | |
| | AGAGAAAGGATTTCTGA | | | | |
| | (SEQ ID NO: 400) | | | | |
| 25 | TGGGAGACCTTGCCTGTGGG | MDTWLVCWAI | GAACCTGAAG | EPEVTQTPSHQ | TCTA |
| | GCCATGGGAGCTCAAAATGC | FSLLKAGLTEPE | TCACCCAGACT | VTQMGQEVILR | ATCA |
| | CCCTCCTTTCCTCCACAGGAC | VTQTPSHQVTQ | CCCAGCCATCA | CVPISNHLYFY | CTTAT |
| | CAGATGCCTGAGCTAGGAAA | MGQEVILRCVPI | GGTCACACAG | WYRQILGQKVE | AC |
| | GGCCTCATTCCTGCTGTGATC | SNHLYFYWYR | ATGGGACAGG | FLVSFYNNEISE | (SEQ |
| | CTGCCATGGATACCTGGCTC | QILGQKVEFLV | AAGTGATCTTG | KSEIFDDQFSVE | ID NO: |
| | GTATGCTGGGCAATTTTTAG | SFYNNEISEKSE | CGCTGTGTCCC | RPDGSNFTLKIR | 408) |
| | TCTCTTGAAAGCAGGACTCA | IFDDQFSVERPD | CATCTCTAATC | STKLEDSAMYF | |
| | CAGAACCTGAAGTCACCCAG | GSNFTLKIRSTK | ACTTATACTTC | CASSVQATGHG | |
| | ACTCCCAGCCATCAGGTCAC | LEDSAMYFCAS | TATTGGTACAG | YTFGSGTRLTV | |
| | ACAGATGGGACAGGAAGTG | SVQATGHGYTF | ACAAATCTTGG | V | |
| | ATCTTGCGCTGTGTCCCCATC | GSGTRLTVVED | GGCAGAAAGT | (SEQ ID NO: | |
| | TCTAATCACTTATACTTCTAT | LNKVFPPEVAV | CGAGTTTCTGG | 653) | |
| | TGGTACAGACAAATCTTGGG | FEPSEAEISHT | TTTCCTTTTAT | | |
| | GCAGAAAGTCGAGTTTCTGG | QKATLVCLAT | AATAATGAAA | | |
| | TTTCCTTTTATAATAATGAAA | GFYPDHVELS | TCTCAGAGAA | | |
| | TCTCAGAGAAGTCTGAAATA | WWVNGKEVH | GTCTGAAATAT | | |
| | TTCGATGATCAATTCTCAGTT | SGVSTDPQPLK | TCGATGATCAA | | |
| | GAAAGGCCTGATGGATCAAA | EQPALNDSRY | TTCTCAGTTGA | | |
| | TTTCACTCTGAAGATCCGGT | CLSSRLRVSAT | AAGGCCTGAT | | |
| | CCACAAAGCTGGAGGACTCA | FWQNPRNHFR | GGATCAAATTT | | |
| | GCCATGTACTTCTGTGCCAG | CQVQFYGLSE | CACTCTGAAGA | | |
| | CAGCGTACAGGCTACGGGCC | NDEWTQDRAK | TCCGGTCCACA | | |
| | ATGGCTACACCTTCGGTTCG | PVTQIVSAEAW | AAGCTGGAGG | | |
| | GGGACCAGGTTAACCGTTGT | GRADCGFTSVS | ACTCAGCCATG | | |
| | AGAGGACCTGAACAAGGTG | YQQGVLSATIL | TACTTCTGTGC | | |
| | TTCCCACCCGAGGTCGCTG | YEILLGKATLY | CAGCAGCGTA | | |
| | TGTTTGAGCCATCAGAAGC | AVLVSALVLM | CAGGCTACGG | | |
| | AGAGATCTCCCACACCCAA | AMVKRKDF | GCCATGGCTAC | | |
| | AAGGCCACACTGGTATGCC | (SEQ ID NO: | ACCTTCGGTTC | | |
| | TGGCCACAGGCTTCTACCC | 651) | GGGGACCAGG | | |
| | CGACCACGTGGAGCTGAGC | | TTAACCGTTGT | | |
| | TGGTGGGTGAATGGGAAG | | AG | | |
| | GAGGTGCACAGTGGGGTC | | (SEQ ID NO: | | |
| | AGCACAGACCCGCAGCCCC | | 652) | | |
| | TCAAGGAGCAGCCCGCCCT | | | | |
| | CAATGACTCCAGATACTGC | | | | |
| | CTGAGCAGCCGCCTGAGG | | | | |
| | GTCTGGCCACCTTCTGGC | | | | |
| | AGAACCCCCGCAACCACTT | | | | |
| | CCGCTGTCAAGTCCAGTTC | | | | |
| | TACGGGCTCTCGGAGAATG | | | | |
| | ACGAGTGGACCCAGGATAG | | | | |
| | GGCCAAACCCGTCACCCAG | | | | |
| | ATCGTCAGCGCCGAGGCCT | | | | |
| | GGGGTAGAGCAGACTGTG | | | | |
| | GCTTTACCTCGGTGTCCTA | | | | |
| | CCAGCAAGGGGTCCTGTCT | | | | |
| | GCCACCATCCTCTATGAGA | | | | |
| | TCCTGCTAGGGAAGGCCAC | | | | |
| | CCTGTATGCTGTGCTGGTC | | | | |
| | AGCGCCCTTGTGTTGATGG | | | | |
| | CCATGGTCAAGAGAAAGGA | | | | |
| | TTTCTGA (SEQ ID NO: 407) | | | | |
| 26 | TGGGGTCACTGTGGGAACTG | MGTRLLCWVV | GGTGCTGGAGT | GAGVSQSPRYK | TCGG |
| | CCCTGTGGAGACAAGGACGG | LGFLGTDHTGA | CTCCCAGTCCC | VAKRGQDVAL | GTCA |
| | CCCTTATCCTCTGCTTCTGTT | GVSQSPRYKVA | CTAGGTACAA | RCDPISGHVSLF | TGTA |
| | CACAGTGACACTGATCTGGT | KRGQDVALRC | AGTCGCAAAG | WYQQALGQGP | TCC |
| | AAAGCCCCCATCCTGGCCTG | DPISGHVSLFW | AGAGGACAGG | EFLTYFQNEAQ | (SEQ |
| | ACCCTGCCATGGGCACCAGG | YQQALGQGPEF | ATGTAGCTCTC | LDKSGLPSDRFF | ID NO: |
| | CTCCTCTGCTGGGTGGTCCTG | LTYFQNEAQLD | AGGTGTGATCC | AERPEGSVSTL | 415) |
| | GGTTTCCTAGGGACAGATCA | KSGLPSDRFFAE | AATTTCGGGTC | KIQRTQQEDSA | |
| | CACAGGTGCTGGAGTCTCCC | RPEGSVSTLKIQ | ATGTATCCCTT | VYLCASTPSGY | |
| | AGTCCCCTAGGTACAAAGTC | RTQQEDSAVYL | TTTTGGTACCA | NSWEQFFGPGT | |
| | GCAAAGAGAGGACAGGATG | CASTPSGYNSW | ACAGGCCCTG | RLTVL | |
| | TAGCTCTCAGGTGTGATCCA | EQFFGPGTRLT | GGGCAGGGGC | (SEQ ID NO: | |
| | ATTTCGGGTCATGTATCCCTT | VLEDLKNVFPP | CAGAGTTTCTG | 656) | |
| | TTTTGGTACCAACAGGCCCT | EVAVFEPSEAE | ACTTATTTCCA | | |
| | GGGGCAGGGGCCAGAGTTTC | ISHTQKATLVC | GAATGAAGCT | | |
| | TGACTTATTTCCAGAATGAA | LATGFYPDHV | CAACTAGACA | | |
| | GCTCAACTAGACAAATCGGG | ELSWWVNGKE | AATCGGGGCT | | |
| | GCTGCCCAGTGATCGCTTCTT | VHSGVSTDPQP | GCCCAGTGATC | | |
| | TGCAGAAAGGCCTGAGGGAT | LKEQPALNDS | GCTTCTTTGCA | | |
| | CCGTCTCCACTCTGAAGATC | RYCLSSRLRVS | GAAAGGCCTG | | |
| | CAGCGCACACAGCAGGAGG | ATFWQNPRNH | AGGGATCCGTC | | |
| | ACTCCGCCGTGTATCTCTGTG | FRCQVQFYGL | TCCACTCTGAA | | |
| | CCAGCACCCCCTCTGGCTAT | SENDEWTQDR | GATCCAGCGC | | |
| | AACTCTTGGGAGCAGTTCTT | AKPVTQIV | ACACAGCAGG | | |
| | CGGGCCAGGGACACGGCTCA | SAEAWGRADC | AGGACTCCGCC | | |
| | CCGTGCTAGAGGACCTGAA | GFTSVSYQQG | GTGTATCTCTG | | |
| | AAACGTGTTCCCACCCGAG | VLSATILYEILL | TGCCAGCACCC | | |
| | GTCGCTGTGTTTGAGCCAT | GKATLYAVLV | CCTCTGGCTAT | | |
| | CAGAAGCAGAGATCTCCCA | SALVLMAMVK | AACTCTTGGGA | | |
| | CACCCAAAAGGCCACACTG | RKDF | GCAGTTCTTCG | | |
| | GTGTGCCTGGCCACAGGCT | (SEQ ID NO: | GGCCAGGGAC | | |
| | TCTACCCCGACCACGTGGA | 654) | ACGGCTCACCG | | |
| | GCTGAGCTGGTGGGTGAAT | | TGCTAG | | |
| | GGGAAGGAGGTGCACAGT | | (SEQ ID NO: | | |
| | GGGGTCAGCACAGACCCG | | 655) | | |
| | CAGCCCCTCAAGGAGCAGC | | | | |
| | CCGCCCTCAATGACTCCAG | | | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCCGTC | | | | |
| | ACCCAGATCGTCAGCGCCG | | | | |
| | AGGCCTGGGGTAGAGCAG | | | | |
| | ACTGTGGCTTTACCTCGGT | | | | |
| | GTCCTACCAGCAAGGGGTC | | | | |
| | CTGTCTGCCACCATCCTCT | | | | |
| | ATGAGATCCTGCTAGGGAA | | | | |
| | GGCCACCCTGTATGCTGTG | | | | |
| | CTGGTCAGCGCCCTTGTGT | | | | |
| | TGATGGCCATGGTCAAGAG | | | | |
| | AAAGGATTTCTGA (SEQ ID | | | | |
| | NO: 414) | | | | |
| 27 | GTGTTGCGTTTGTACACTTCA | MGPQLLGYVV | GAAGCCCAAG | EAQVTQNPRYL | ATGA |
| | GCCCTCTGAGCTGAAGTGGG | LCLLGAGPLEA | TGACCCAGAA | ITVTGKKLTVT | ACCA |
| | AGTGGTTTCTCTCCTGAAAA | QVTQNPRYLIT | CCCAAGATACC | CSQNMNHEYM | TGAG |
| | TGTTTCTGAGGCCCAAATAG | VTGKKLTVTCS | TCATCACAGTG | SWYRQDPGLGL | TAT |
| | CTGAAGAGGTGGAGACGTTA | QNMNHEYMSW | ACTGGAAAGA | RQIYYSMNVEV | (SEQ |
| | CAGAAACCACCTGGAGCCCC | YRQDPGLGLRQ | AGTTAACAGTG | TDKGDVPEGYK | ID NO: |
| | CAGAACTGGCAGACACCTGC | IYYSMNVEVTD | ACTTGTTCTCA | VSRKEKRNFPLI | 422) |
| | CTGATGCTGCCATGGGCCCC | KGDVPEGYKVS | GAATATGAAC | LESPSPNQTSLY | |
| | CAGCTCCTTGGCTATGTGGT | RKEKRNFPLILE | CATGAGTATAT | FCATAPRGSNQ | |
| | CCTTTGCCTTCTAGGAGCAG | SPSPNQTSLYFC | GTCCTGGTATC | PQHFGDGTRLSI | |
| | GCCCCCTGGAAGCCCAAGTG | ATAPRGSNQPQ | GACAAGACCC | L | |
| | ACCCAGAACCCAAGATACCT | HFGDGTRLSILE | AGGGCTGGGC | (SEQ ID NO: | |
| | CATCACAGTGACTGGAAAGA | DLNKVFPPEVA | TTAAGGCAGAT | 659) | |
| | AGTTAACAGTGACTTGTTCT | VFEPSEAEISH | CTACTATTCAA | | |
| | CAGAATATGAACCATGAGTA | TQKATLVCLA | TGAATGTTGAG | | |
| | TATGTCCTGGTATCGACAAG | TGFYPDHVELS | GTGACTGATAA | | |
| | ACCCAGGGCTGGGCTTAAGG | WWVNGKEVH | GGGAGATGTTC | | |
| | CAGATCTACTATTCAATGAA | SGVSTDPQPLK | CTGAAGGGTA | | |
| | TGTTGAGGTGACTGATAAGG | EQPALNDSRY | CAAAGTCTCTC | | |
| | GAGATGTTCCTGAAGGGTAC | CLSSRLRVSAT | GAAAAGAGAA | | |
| | AAAGTCTCTCGAAAAGAGAA | FWQNPRNHFR | GAGGAATTTCC | | |
| | GAGGAATTTCCCCCTGATCC | CQVQFYGLSE | CCCTGATCCTG | | |
| | TGGAGTCGCCCAGCCCCAAC | NDEWTQDRAK | GAGTCGCCCA | | |
| | CAGACCTCTCTGTACTTCTGT | PVTQIVSAEAW | GCCCCAACCA | | |
| | GCCACCGCCCCCAGGGGTAG | GRADCGFTSVS | GACCTCTCTGT | | |
| | CAATCAGCCCCAGCATTTTG | YQQGVLSATIL | ACTTCTGTGCC | | |
| | GTGATGGGACTCGACTCTCC | YEILLGKATLY | ACCGCCCCCAG | | |
| | ATCCTAGAGGACCTGAACA | AVLVSALVLM | GGGTAGCAAT | | |
| | AGGTGTTCCCACCCGAGGT | AMVKRKDF | CAGCCCCAGC | | |
| | CGCTGTGTTTGAGCCATCA | (SEQ ID NO: | ATTTTGGTGAT | | |
| | GAAGCAGAGATCTCCCACA | 657) | GGGACTCGACT | | |
| | CCCAAAAGGCCACACTGGT | | CTCCATCCTAG | | |
| | GTGCCTGGCCACAGGCTTC | | (SEQ ID NO: | | |
| | TACCCCGACCACGTGGAGC | | 658) | | |
| | TGAGCTGGTGGGTGAATGG | | | | |
| | GAAGGAGGTGCACAGTGG | | | | |
| | GGTCAGCACAGACCCGCAG | | | | |
| | CCCCTCAAGGAGCAGCCCG | | | | |
| | CCCTCAATGACTCCAGATA | | | | |
| | CTGCCTGAGCAGCCGCCTG | | | | |
| | AGGGTCTCGGCCACCTTCT | | | | |
| | GGCAGAACCCCCGCAACCA | | | | |
| | CTTCCGCTGTCAAGTCCAG | | | | |
| | TTCTACGGGCTCTCGGAGA | | | | |
| | ATGACGAGTGGACCCAGGA | | | | |
| | TAGGGCCAAACCCGTCACC | | | | |
| | CAGATCGTCAGCGCCGAGG | | | | |
| | CCTGGGGTAGAGCAGACTG | | | | |
| | TGGCTTTACCTCGGTGTCC | | | | |
| | TACCAGCAAGGGGTCCTGT | | | | |
| | CTGCCACCATCCTCTATGA | | | | |
| | GATCCTGCTAGGGAAGGCC | | | | |
| | ACCCTGTATGCTGTGCTGG | | | | |
| | TCAGCGCCCTTGTGTTGAT | | | | |
| | GGCCATGGTCAAGAGAAAG | | | | |
| | GATTTCTGA (SEQ ID NO: | | | | |
| | 421) | | | | |
| 28 | GGGGGGGATTCTGTGATCAG | MEAVVTTLPRE | GGTGCTGTCGT | GAVVSQHPSW | GACT |
| | TCATCCCTCCTCGCTGGTGA | GGVRPSRKMLL | CTCTCAACATC | VICKSGTSVKIE | TTCA |
| | ATGGAGGCAGTGGTCACAAC | LLLLLGPGSGL | CGAGCTGGGTT | CRSLDFQATTM | GGCC |
| | TCTCCCCAGAGAAGGTGGTG | GAVVSQHPSW | ATCTGTAAGAG | FWYRQFPKQSL | ACAA |
| | TGAGGCCATCACGGAAGATG | VICKSGTSVKIE | TGGAACCTCTG | MLMATSNEGS | CT |
| | CTGCTGCTTCTGCTGCTTCTG | CRSLDFQATTM | TGAAGATCGA | KATYEQGVEK | (SEQ |
| | GGGCCAGGCTCCGGGCTTGG | FWYRQFPKQSL | GTGCCGTTCCC | DKFLINHASLTL | ID NO: |
| | TGCTGTCGTCTCTCAACATCC | MLMATSNEGS | TGGACTTTCAG | STLTVTSAHPE | 429) |
| | GAGCTGGGTTATCTGTAAGA | KATYEQGVEK | GCCACAACTAT | DSSFYICRAWG | |
| | GTGGAACCTCTGTGAAGATC | DKFLINHASLTL | GTTTTGGTATC | GSSYNEQFFGP | |
| | GAGTGCCGTTCCCTGGACTT | STLTVTSAHPE | GTCAGTTCCCG | GTRLTVL | |
| | TCAGGCCACAACTATGTTTT | DSSFYICRAWG | AAACAGAGTC | (SEQ ID NO: | |
| | GGTATCGTCAGTTCCCGAAA | GSSYNEQFFGP | TCATGCTGATG | 662) | |
| | CAGAGTCTCATGCTGATGGC | GTRLTVLEDLK | GCAACTTCCAA | | |
| | AACTTCCAATGAGGGCTCCA | NVFPPEVAVFE | TGAGGGCTCCA | | |
| | AGGCCACATACGAGCAAGGC | PSEAEISHTQK | AGGCCACATA | | |
| | GTCGAGAAGGACAAGTTTCT | ATLVCLATGF | CGAGCAAGGC | | |
| | CATCAACCATGCAAGCCTGA | YPDHVELSWW | GTCGAGAAGG | | |
| | CCTTGTCCACTCTGACAGTG | VNGKEVHSGV | ACAAGTTTCTC | | |
| | ACCAGTGCCCATCCTGAAGA | STDPQPLKEQP | ATCAACCATGC | | |
| | CAGCAGCTTCTACATCTGCA | ALNDSRYCLSS | AAGCCTGACCT | | |
| | GGGCCTGGGGCGGGAGCTCC | RLRVSATFWQ | TGTCCACTCTG | | |
| | TACAATGAGCAGTTCTTCGG | NPRNHFRCQV | ACAGTGACCA | | |
| | GCCAGGGACACGGCTCACCG | QFYGLSENDE | GTGCCCATCCT | | |
| | TGCTAGAGGACCTGAAAAA | WTQDRAKPVT | GAAGACAGCA | | |
| | CGTGTTCCCACCCGAGGTC | QIVSAEAWGR | GCTTCTACATC | | |
| | GCTGTGTTTGAGCCATCAG | ADCGFTSVSYQ | TGCAGGGCCTG | | |
| | AAGCAGAGATCTCCCACAC | QGVLSATILYE | GGGCGGGAGC | | |
| | CCAAAAGGCCACACTGGTA | ILLGKATLYAV | TCCTACAATGA | | |
| | TGCCTGGCCACAGGCTTCT | LVSALVLMAM | GCAGTTCTTCG | | |
| | ACCCCGACCACGTGGAGCT | VKRKDF | GGCCAGGGAC | | |
| | GAGCTGGTGGGTGAATGG | (SEQ ID NO: | ACGGCTCACCG | | |
| | GAAGGAGGTGCACAGTGG | 660) | TGCTAG | | |
| | GGTCAGCACAGACCCGCAG | | (SEQ ID NO: | | |
| | CCCCTCAAGGAGCAGCCCG | | 661) | | |
| | CCCTCAATGACTCCAGATA | | | | |
| | CTGCCTGAGCAGCCGCCTG | | | | |
| | AGGGTCTCGGCCACCTTCT | | | | |
| | GGCAGAACCCCCGCAACCA | | | | |
| | CTTCCGCTGTCAAGTCCAG | | | | |
| | TTCTACGGGCTCTCGGAGA | | | | |
| | ATGACGAGTGGACCCAGGA | | | | |
| | TAGGGCCAAACCCGTCACC | | | | |
| | CAGATCGTCAGCGCCGAGG | | | | |
| | CCTGGGGTAGAGCAGACTG | | | | |
| | TGGCTTTACCTCGGTGTCC | | | | |
| | TACCAGCAAGGGGTCCTGT | | | | |
| | CTGCCACCATCCTCTATGA | | | | |
| | GATCCTGCTAGGGAAGGCC | | | | |
| | ACCCTGTATGCTGTGCTGG | | | | |
| | TCAGCGCCCTTGTGTTGAT | | | | |
| | GGCCATGGTCAAGAGAAAG | | | | |
| | GATTTCTGA (SEQ ID NO: | | | | |
| | 428) | | | | |
| 29 | TGGGGAGTCATCCCTCCTCG | MEAVVTTLPRE | GGTGCTGTCGT | GAVVSQHPSRV | GACT |
| | CTGGTGAATGGAGGCAGTGG | GGVRPSRKMLL | CTCTCAACATC | ICKSGTSVKIEC | TTCA |
| | TCACAACTCTCCCCAGAGAA | LLLLLGPGSGL | CGAGCAGGGT | RSLDFQATTMF | GGCC |
| | GGTGGTGTGAGGCCATCACG | GAVVSQHPSRV | TATCTGTAAGA | WYRQFPKQSL | ACAA |
| | GAAGATGCTGCTGCTTCTGC | ICKSGTSVKIEC | GTGGAACCTCT | MLMATSNEGS | CT |
| | TGCTTCTGGGGCCAGGCTCC | RSLDFQATTMF | GTGAAGATCG | KATYEQGVEK | (SEQ |
| | GGGCTTGGTGCTGTCGTCTCT | WYRQFPKQSL | AGTGCCGTTCC | DKFLINHASLTL | ID NO: |
| | CAACATCCGAGCAGGGTTAT | MLMATSNEGS | CTGGACTTTCA | STLTVTSAHPE | 436) |
| | CTGTAAGAGTGGAACCTCTG | KATYEQGVEK | GGCCACAACT | DSSFYICSARIG | |
| | TGAAGATCGAGTGCCGTTCC | DKFLINHASLTL | ATGTTTTGGTA | QGFMNEQFFGP | |
| | CTGGACTTTCAGGCCACAAC | STLTVTSAHPE | TCGTCAGTTCC | GTRLTVL | |
| | TATGTTTTGGTATCGTCAGTT | DSSFYICSARIG | CGAAACAGAG | (SEQ ID NO: | |
| | CCCGAAACAGAGTCTCATGC | QGFMNEQFFGP | TCTCATGCTGA | 665) | |
| | TGATGGCAACTTCCAATGAG | GTRLTVLEDLK | TGGCAACTTCC | | |
| | GGCTCCAAGGCCACATACGA | NVFPPEVAVFE | AATGAGGGCT | | |
| | GCAAGGCGTCGAGAAGGAC | PSEAEISHTQK | CCAAGGCCAC | | |
| | AAGTTTCTCATCAACCATGC | ATLVCLATGF | ATACGAGCAA | | |
| | AAGCCTGACCTTGTCCACTC | YPDHVELSWW | GGCGTCGAGA | | |
| | TGACAGTGACCAGTGCCCAT | VNGKEVHSGV | AGGACAAGTTT | | |
| | CCTGAAGACAGCAGCTTCTA | STDPQPLKEQP | CTCATCAACCA | | |
| | CATCTGCAGTGCTAGAATTG | ALNDSRYCLSS | TGCAAGCCTGA | | |
| | GACAGGGTTTTATGAATGAG | RLRVSATFWQ | CCTTGTCCACT | | |
| | CAGTTCTTCGGGCCAGGGAC | NPRNHFRCQV | CTGACAGTGAC | | |
| | ACGGCTCACCGTGCTAGAGG | QFYGLSENDE | CAGTGCCCATC | | |
| | ACCTGAAAAACGTGTTCCC | WTQDRAKPVT | CTGAAGACAG | | |
| | ACCCGAGGTCGCTGTGTTT | QIVSAEAWGR | CAGCTTCTACA | | |
| | GAGCCATCAGAAGCAGAGA | ADCGFTSESYQ | TCTGCAGTGCT | | |
| | TCTCCCACACCCAAAAGGC | QGVLSATILYE | AGAATTGGAC | | |
| | CACACTGGTGTGCCTGGCC | ILLGKATLYAV | AGGGTTTTATG | | |
| | ACAGGCTTCTACCCCGACC | LVSALVLMAM | AATGAGCAGTT | | |
| | ACGTGGAGCTGAGCTGGTG | VKRKDSRG | CTTCGGGCCAG | | |
| | GGTGAATGGGAAGGAGGT | (SEQ ID NO: | GGACACGGCT | | |
| | GCACAGTGGGGTCAGCACA | 663) | CACCGTGCTAG | | |
| | GACCCGCAGCCCCTCAAGG | | (SEQ ID NO: | | |
| | AGCAGCCCGCCCTCAATGA | | 664) | | |
| | CTCCAGATACTGCCTGAGC | | | | |
| | AGCCGCCTGAGGGTCTCGG | | | | |
| | CCACCTTCTGGCAGAACCC | | | | |
| | CCGCAACCACTTCCGCTGT | | | | |
| | CAAGTCCAGTTCTACGGGC | | | | |
| | TCTCGGAGAATGACGAGTG | | | | |
| | GACCCAGGATAGGGCCAAA | | | | |
| | CCTGTCACCCAGATCGTCA | | | | |
| | GCGCCGAGGCCTGGGGTA | | | | |
| | GAGCAGACTGTGGCTTCAC | | | | |
| | CTCCGAGTCTTACCAGCAA | | | | |
| | GGGGTCCTGTCTGCCACCA | | | | |
| | TCCTCTATGAGATCTTGCT | | | | |
| | AGGGAAGGCCACCTTGTAT | | | | |
| | GCCGTGCTGGTCAGTGCCC | | | | |
| | TCGTGCTGATGGCCATGGT | | | | |
| | CAAGAGAAAGGATTCCAGA | | | | |
| | GGCTAG (SEQ ID NO: 435) | | | | |
| 30 | AGTCATGGGCAAAGATTACC | MEAVVTTLPRE | GGTGCTGTCGT | GAVVSQHPSRV | GACT |
| | ACCAGGGGGCAGACTAGGG | GGVRPSRKMLL | CTCTCAACATC | ICKSGTSVKIEC | TTCA |
| | CATCCTTGGGATTCTGTGATC | LLLLLGPGSGL | CGAGCAGGGT | RSLDFQATTMF | GGCC |
| | AGTCATCCCTCCTCGCTGGT | GAVVSQHPSRV | TATCTGTAAGA | WYRQFPKKSL | ACAA |
| | GAATGGAGGCAGTGGTCACA | ICKSGTSVKIEC | GTGGAACCTCT | MLMATSNEGS | CT |
| | ACTCTCCCCAGAGAAGGTGG | RSLDFQATTMF | GTGAAGATCG | KATYEQGVEK | (SEQ |
| | TGTGAGGCCATCACGGAAGA | WYRQFPKKSL | AGTGCCGTTCC | DKFLINHASLTL | ID NO: |
| | TGCTGCTGCTTCTGCTGCTTC | MLMATSNEGS | CTGGACTTTCA | STLTVTSAHPE | 443) |
| | TGGGGCCAGGCTCCGGGCTT | KATYEQGVEK | GGCCACAACT | DSSFYICSAPG | |
| | GGTGCTGTCGTCTCTCAACA | DKFLINHASLTL | ATGTTTTGGTA | WRGTEAFFGQG | |
| | TCCGAGCAGGGTTATCTGTA | STLTVTSAHPE | TCGTCAGTTCC | TRLTVV | |
| | AGAGTGGAACCTCTGTGAAG | DSSFYICSAPG | CGAAAAAGAG | (SEQ ID NO: | |
| | ATCGAGTGCCGTTCCCTGGA | WRGTEAFFGQG | TCTCATGCTGA | 668) | |
| | CTTTCAGGCCACAACTATGT | TRLTVVEDLNK | TGGCAACTTCC | | |
| | TTTGGTATCGTCAGTTCCCGA | VFPPEVAVFEP | AATGAGGGCT | | |
| | AAAAGAGTCTCATGCTGATG | SEAEISHTQKA | CCAAGGCCAC | | |
| | GCAACTTCCAATGAGGGCTC | TLVCLATGFFP | ATACGAGCAA | | |
| | CAAGGCCACATACGAGCAAG | DHVELSWWVN | GGCGTCGAGA | | |
| | GCGTCGAGAAGGACAAGTTT | GKEVHSGVST | AGGACAAGTTT | | |
| | CTCATCAACCATGCAAGCCT | DPQPLKEQPA | CTCATCAACCA | | |
| | GACCTTGTCCACTCTGACAG | LNDSRYCLSSR | TGCAAGCCTGA | | |
| | TGACCAGTGCCCATCCTGAA | LRVSATFWQN | CCTTGTCCACT | | |
| | GACAGCAGCTTCTACATCTG | PRNHFRCQVQ | CTGACAGTGAC | | |
| | CAGTGCTCCTGGGTGGCGTG | FYGLSENDEW | CAGTGCCCATC | | |
| | GCACTGAAGCTTTCTTTGGA | TQDRAKPVTQI | CTGAAGACAG | | |
| | CAAGGCACCAGACTCACAGT | VSAEAWGRAD | CAGCTTCTACA | | |
| | TGTAGAGGACCTGAACAAG | CGFTSVSYQQ | TCTGCAGTGCT | | |
| | GTGTTCCCACCCGAGGTCG | GVLSATILYEI | CCTGGGTGGCG | | |
| | CTGTGTTTGAGCCATCAGA | LLGKATLYAV | TGGCACTGAA | | |
| | AGCAGAGATCTCCCACACC | LVSALVLMAM | GCTTTCTTTGG | | |
| | CAAAAGGCCACACTGGTGT | VKRKDF | ACAAGGCACC | | |
| | GCCTGGCCACAGGCTTCTT | (SEQ ID NO: | AGACTCACAGT | | |
| | CCCTGACCACGTGGAGCTG | 666) | TGTAG | | |
| | AGCTGGTGGGTGAATGGG | | (SEQ ID NO: | | |
| | AAGGAGGTGCACAGTGGG | | 667) | | |
| | GTCAGCACAGACCCGCAGC | | | | |
| | CCCTCAAGGAGCAGCCCGC | | | | |
| | CCTCAATGACTCCAGATAC | | | | |
| | TGCCTGAGCAGCCGCCTGA | | | | |
| | GGGTCTCGGCCACCTTCTG | | | | |
| | GCAGAACCCCCGCAACCAC | | | | |
| | TTCCGCTGTCAAGTCCAGT | | | | |
| | TCTACGGGCTCTCGGAGAA | | | | |
| | TGACGAGTGGACCCAGGAT | | | | |
| | AGGGCCAAACCCGTCACCC | | | | |
| | AGATCGTCAGCGCCGAGGC | | | | |
| | CTGGGGTAGAGCAGACTGT | | | | |
| | GGCTTTACCTCGGTGTCCT | | | | |
| | ACCAGCAAGGGGTCCTGTC | | | | |
| | TGCCACCATCCTCTATGAG | | | | |
| | ATCCTGCTAGGGAAGGCCA | | | | |
| | CCCTGTATGCTGTGCTGGT | | | | |
| | CAGCGCCCTTGTGTTGATG | | | | |
| | GCCATGGTCAAGAGAAAGG | | | | |
| | ATTTCTGA (SEQ ID NO: 442) | | | | |
| 31 | GGGACTGGCGCAGCACCTCT | MGCRLLCCVVF | GACACAGCTGT | DTAVSQTPKYL | CTGG |
| | CAGCGGCAGTGGAAACCACA | CLLQAGPLDTA | TTCCCAGACTC | VTQMGNDKSIK | GCCA |
| | GCCTAGTCCTCTCACCACTG | VSQTPKYLVTQ | CAAAATACCTG | CEQNLGHDTM | TGAT |
| | CAGACCAGAATCCTGCCCTG | MGNDKSIKCEQ | GTCACACAGAT | YWYKQDSKKF | ACT |
| | GGCCTTGCCTGGTCTGCCTC | NLGHDTMYWY | GGGAAACGAC | LKIMFSYNNKE | (SEQ |
| | ACTCTGCCATGGGCTGCAGG | KQDSKKFLKIM | AAGTCCATTAA | LIINETVPNRFSP | ID NO: |
| | CTCCTCTGCTGTGTGGTCTTC | FSYNNKELIINE | ATGTGAACAA | KSPDKAHLNLH | 450) |
| | TGCCTCCTCCAAGCAGGTCC | TVPNRFSPKSPD | AATCTGGGCCA | INSLELGDSAV | |
| | CTTGGACACAGCTGTTTCCC | KAHLNLHINSL | TGATACTATGT | YFCASSQDGLE | |
| | AGACTCCAAAATACCTGGTC | ELGDSAVYFCA | ATTGGTATAAA | QYFGPGTRLTV | |
| | ACACAGATGGGAAACGACA | SSQDGLEQYFG | CAGGACTCTAA | T | |
| | AGTCCATTAAATGTGAACAA | PGTRLTVTEDL | GAAATTTCTGA | (SEQ ID NO: | |
| | AATCTGGGCCATGATACTAT | KNVFPPEVAVF | AGATAATGTTT | 671) | |
| | GTATTGGTATAAACAGGACT | EPSEAEISHTQ | AGCTACAATA | | |
| | CTAAGAAATTTCTGAAGATA | KATLVCLATG | ATAAGGAGCT | | |
| | ATGTTTAGCTACAATAATAA | FYPDHVELSW | CATTATAAATG | | |
| | GGAGCTCATTATAAATGAAA | WVNGKEVHSG | AAACAGTTCCA | | |
| | CAGTTCCAAATCGCTTCTCA | VSTDPQPLKEQ | AATCGCTTCTC | | |
| | CCTAAATCTCCAGACAAAGC | PALNDSRYCLS | ACCTAAATCTC | | |
| | TCACTTAAATCTTCACATCA | SRLRVSATFW | CAGACAAAGC | | |
| | ATTCCCTGGAGCTTGGTGAC | QNPRNHFRCQ | TCACTTAAATC | | |
| | TCTGCTGTGTATTTCTGTGCC | VQFYGLSEND | TTCACATCAAT | | |
| | AGCAGCCAAGATGGACTTGA | EWTQDRAKPV | TCCCTGGAGCT | | |
| | GCAGTACTTCGGGCCGGGCA | TQIVSAEAWG | TGGTGACTCTG | | |
| | CCAGGCTCACGGTCACAGAG | RADCGFTSESY | CTGTGTATTTC | | |
| | GACCTGAAAAACGTGTTCC | QQGVLSATILY | TGTGCCAGCAG | | |
| | CACCCGAGGTCGCTGTGTT | EILLGKATLYA | CCAAGATGGA | | |
| | TGAGCCATCAGAAGCAGAG | VLVSALVLMA | CTTGAGCAGTA | | |
| | ATCTCCCACACCCAAAAGG | MVKRKDSRG | CTTCGGGCCGG | | |
| | CCACACTGGTATGCCTGGC | (SEQ ID NO: | GCACCAGGCTC | | |
| | CACAGGCTTCTACCCCGAC | 669) | ACGGTCACAG | | |
| | CACGTGGAGCTGAGCTGGT | | (SEQ ID NO: | | |
| | GGGTGAATGGGAAGGAGG | | 670) | | |
| | TGCACAGTGGGGTCAGCAC | | | | |
| | AGACCCGCAGCCCCTCAAG | | | | |
| | GAGCAGCCCGCCCTCAATG | | | | |
| | ACTCCAGATACTGCCTGAG | | | | |
| | CAGCCGCCTGAGGGTCTCG | | | | |
| | GCCACCTTCTGGCAGAACC | | | | |
| | CCCGCAACCACTTCCGCTG | | | | |
| | TCAAGTCCAGTTCTACGGG | | | | |
| | CTCTCGGAGAATGACGAGT | | | | |
| | GGACCCAGGATAGGGCCA | | | | |
| | AACCTGTCACCCAGATCGT | | | | |
| | CAGCGCCGAGGCCTGGGG | | | | |
| | TAGAGCAGACTGTGGCTTC | | | | |
| | ACCTCCGAGTCTTACCAGC | | | | |
| | AAGGGGTCCTGTCTGCCAC | | | | |
| | CATCCTCTATGAGATCTTG | | | | |
| | CTAGGGAAGGCCACCTTGT | | | | |
| | ATGCCGTGCTGGTCAGTGC | | | | |
| | CCTCGTGCTGATGGCCATG | | | | |
| | GTCAAGAGAAAGGATTCCA | | | | |
| | GAGGCTAG (SEQ ID NO: 449) | | | | |
| 32 | GACCTCACTGGCGCAGCACC | MGCRLLCCVVF | GACACAGCTGT | DTAVSQTPKYL | CTGG |
| | TCTCAGCGGCAGTGGAAACC | CLLQAGPLDTA | TTCCCAGACTC | VTQMGNDKSIK | GCCA |
| | ACAGCCTAGTCCTCTCACCA | VSQTPKYLVTQ | CAAAATACCTG | CEQNLGHDTM | TGAT |
| | CTGCAGACCAGAATCCTGCC | MGNDKSIKCEQ | GTCACACAGAT | YWYKQDSKKF | ACT |
| | CTGGGCCTTGCCTGGTCTGC | NLGHDTMYWY | GGGAAACGAC | LKIMFSYNNKE | (SEQ |
| | CTCACTCTGCCATGGGCTGC | KQDSKKFLKIM | AAGTCCATTAA | LIINETVPNRFSP | ID NO: |
| | AGGCTCCTCTGCTGTGTGGT | FSYNNKELIINE | ATGTGAACAA | KSPDKAHLNLH | 457) |
| | CTTCTGCCTCCTCCAAGCAG | TVPNRFSPKSPD | AATCTGGGCCA | INSLELGDSAV | |
| | GTCCCTTGGACACAGCTGTT | KAHLNLHINSL | TGATACTATGT | YFCASSQEGSQ | |
| | TCCCAGACTCCAAAATACCT | ELGDSAVYFCA | ATTGGTATAAA | DHTSGRATEQY | |
| | GGTCACACAGATGGGAAACG | SSQEGSQDHTS | CAGGACTCTAA | FGPGTRLTVT | |
| | ACAAGTCCATTAAATGTGAA | GRATEQYFGPG | GAAATTTCTGA | (SEQ ID NO: | |
| | CAAAATCTGGGCCATGATAC | TRLTVTEDLKN | AGATAATGTTT | 674) | |
| | TATGTATTGGTATAAACAGG | VFPPEVAVFEP | AGCTACAATA | | |
| | ACTCTAAGAAATTTCTGAAG | SEAEISHTQKA | ATAAGGAGCT | | |
| | ATAATGTTTAGCTACAATAA | TLVCLATGFYP | CATTATAAATG | | |
| | TAAGGAGCTCATTATAAATG | DHVELSWWVN | AAACAGTTCCA | | |
| | AAACAGTTCCAAATCGCTTC | GKEVHSGVST | AATCGCTTCTC | | |
| | TCACCTAAATCTCCAGACAA | DPQPLKEQPA | ACCTAAATCTC | | |
| | AGCTCACTTAAATCTTCACA | LNDSRYCLSSR | CAGACAAAGC | | |
| | TCAATTCCCTGGAGCTTGGT | LRVSATFWQN | TCACTTAAATC | | |
| | GACTCTGCTGTGTATTTCTGT | PRNHFRCQVQ | TTCACATCAAT | | |
| | GCCAGCAGCCAAGAAGGATC | FYGLSENDEW | TCCCTGGAGCT | | |
| | TCAGGACCACACTAGCGGGA | TQDRAKPVTQI | TGGTGACTCTG | | |
| | GGGCCACCGAGCAGTACTTC | VSAEAWGRAD | CTGTGTATTTC | | |
| | GGGCCGGGCACCAGGCTCAC | CGFTSESYQQ | TGTGCCAGCAG | | |
| | GGTCACAGAGGACCTGAAA | GVLSATILYEI | CCAAGAAGGA | | |
| | AACGTGTTCCCACCCGAGG | LLGKATLYAV | TCTCAGGACCA | | |
| | TCGCTGTGTTTGAGCCATC | LVSALVLMAM | CACTAGCGGG | | |
| | AGAAGCAGAGATCTCCCAC | VKRKDSRG | AGGGCCACCG | | |
| | ACCCAAAAGGCCACACTGG | (SEQ ID NO: | AGCAGTACTTC | | |
| | TGTGCCTGGCCACAGGCTT | 672) | GGGCCGGGCA | | |
| | CTACCCCGACCACGTGGAG | | CCAGGCTCACG | | |
| | CTGAGCTGGTGGGTGAATG | | GTCACAG | | |
| | GGAAGGAGGTGCACAGTG | | (SEQ ID NO: | | |
| | GGGTCAGCACAGACCCGCA | | 673) | | |
| | GCCCCTCAAGGAGCAGCCC | | | | |
| | GCCCTCAATGACTCCAGAT | | | | |
| | ACTGCCTGAGCAGCCGCCT | | | | |
| | GAGGGTCTCGGCCACCTTC | | | | |
| | TGGCAGAACCCCCGCAACC | | | | |
| | ACTTCCGCTGTCAAGTCCA | | | | |
| | GTTCTACGGGCTCTCGGAG | | | | |
| | AATGACGAGTGGACCCAGG | | | | |
| | ATAGGGCCAAACCTGTCAC | | | | |
| | CCAGATCGTCAGCGCCGAG | | | | |
| | GCCTGGGGTAGAGCAGACT | | | | |
| | GTGGCTTCACCTCCGAGTC | | | | |
| | TTACCAGCAAGGGGTCCTG | | | | |
| | TCTGCCACCATCCTCTATG | | | | |
| | AGATCTTGCTAGGGAAGGC | | | | |
| | CACCTTGTATGCCGTGCTG | | | | |
| | GTCAGTGCCCTCGTGCTGA | | | | |
| | TGGCCATGGTCAAGAGAAA | | | | |
| | GGATTCCAGAGGCTAG | | | | |
| | (SEQ ID NO: 456) | | | | |
| 33 | GGGGCTCTCCCTCCCTGGGG | MSQNDFLESPA | AATGCTGGTGT | NAGVTQTPKFQ | ATGA |
| | CCCAGGCAGGGAGAATGTCT | PLSSMHRYRRP | CACTCAGACCC | VLKTGQSMTLQ | ACCA |
| | CAGAATGACTTCCTTGAGAG | LRHAASAMSIG | CAAAATTCCAG | CAQDMNHEYM | TGAA |
| | TCCTGCTCCCCTTTCATCAAT | LLCCAALSLLW | GTCCTGAAGAC | SWYRQDPGMG | TAC |
| | GCACAGATACAGAAGACCCC | AGPVNAGVTQT | AGGACAGAGC | LRLIHYSVGAGI | (SEQ |
| | TCCGTCATGCAGCATCTGCC | PKFQVLKTGQS | ATGACACTGCA | TDQGEVPNGYN | ID NO: |
| | ATGAGCATCGGCCTCCTGTG | MTLQCAQDMN | GTGTGCCCAGG | VSRSTTEDFPLR | 464) |
| | CTGTGCAGCCTTGTCTCTCCT | HEYMSWYRQD | ATATGAACCAT | LLSAAPSQTSV | |
| | GTGGGCAGGTCCAGTGAATG | PGMGLRLIHYS | GAATACATGTC | YFCASSLTEGRS | |
| | CTGGTGTCACTCAGACCCCA | VGAGITDQGEV | CTGGTATCGAC | EQYFGPGTRLT | |
| | AAATTCCAGGTCCTGAAGAC | PNGYNVSRSTT | AAGACCCAGG | VT | |
| | AGGACAGAGCATGACACTGC | EDFPLRLLSAAP | CATGGGGCTG | (SEQ ID NO: | |
| | AGTGTGCCCAGGATATGAAC | SQTSVYFCASSL | AGGCTGATTCA | 677) | |
| | CATGAATACATGTCCTGGTA | TEGRSEQYFGP | TTACTCAGTTG | | |
| | TCGACAAGACCCAGGCATGG | GTRLTVTEDLK | GTGCTGGTATC | | |
| | GGCTGAGGCTGATTCATTAC | NVFPPEVAVFE | ACTGACCAAG | | |
| | TCAGTTGGTGCTGGTATCAC | PSEAEISHTQK | GAGAAGTCCC | | |
| | TGACCAAGGAGAAGTCCCCA | ATLVCLATGF | CAATGGCTACA | | |
| | ATGGCTACAATGTCTCCAGA | YPDHVELSWW | ATGTCTCCAGA | | |
| | TCAACCACAGAGGATTTCCC | VNGKEVHSGV | TCAACCACAG | | |
| | GCTCAGGCTGCTGTCGGCTG | STDPQPLKEQP | AGGATTTCCCG | | |
| | CTCCCTCCCAGACATCTGTGT | ALNDSRYCLSS | CTCAGGCTGCT | | |
| | ACTTCTGTGCCAGCAGCCTG | RLRVSATFWQ | GTCGGCTGCTC | | |
| | ACAGAAGGAAGAAGCGAGC | NPRNHFRCQV | CCTCCCAGACA | | |
| | AGTACTTCGGGCCGGGCACC | QFYGLSENDE | TCTGTGTACTT | | |
| | AGGCTCACGGTCACAGAGGA | WTQDRAKPVT | CTGTGCCAGCA | | |
| | CCTGAAAAACGTGTTCCCA | QIVSAEAWGR | GCCTGACAGA | | |
| | CCCGAGGTCGCTGTGTTTG | ADCGFTSESYQ | AGGAAGAAGC | | |
| | AGCCATCAGAAGCAGAGAT | QGVLSATILYE | GAGCAGTACTT | | |
| | CTCCCACACCCAAAAGGCC | ILLGKATLYAV | CGGGCCGGGC | | |
| | ACACTGGTGTGCCTGGCCA | LVSALVLMAM | ACCAGGCTCAC | | |
| | CAGGCTTCTACCCCGACCA | VKRKDSRG | GGTCACAG | | |
| | CGTGGAGCTGAGCTGGTG | (SEQ ID NO: | (SEQ ID NO: | | |
| | GGTGAATGGGAAGGAGGT | 675) | 676) | | |
| | GCACAGTGGGGTCAGCAC | | | | |
| | GGACCCGCAGCCCCTCAAG | | | | |
| | GAGCAGCCCGCCCTCAATG | | | | |
| | ACTCCAGATACTGCCTGAG | | | | |
| | CAGCCGCCTGAGGGTCTCG | | | | |
| | GCCACCTTCTGGCAGAACC | | | | |
| | CCCGCAACCACTTCCGCTG | | | | |
| | TCAAGTCCAGTTCTACGGG | | | | |
| | CTCTCGGAGAATGACGAGT | | | | |
| | GGACCCAGGATAGGGCCA | | | | |
| | AACCTGTCACCCAGATCGT | | | | |
| | CAGCGCCGAGGCCTGGGG | | | | |
| | TAGAGCAGACTGTGGCTTC | | | | |
| | ACCTCCGAGTCTTACCAGC | | | | |
| | AAGGGGTCCTGTCTGCCAC | | | | |
| | CATCCTCTATGAGATCTTG | | | | |
| | CTAGGGAAGGCCACCTTGT | | | | |
| | ATGCCGTGCTGGTCAGTGC | | | | |
| | CCTCGTGCTGATGGCCATG | | | | |
| | GTCAAGAGAAAGGATTCCA | | | | |
| | GAGGCTAG (SEQ ID NO: 463) | | | | |
| 34 | GGGGAGTCATCCCTCCTCGC | MEAVVTTLPRE | GGTGCTGTCGT | GAVVSQHPSRV | GACT |
| | TGGTGAATGGAGGCAGTGGT | GGVRPSRKMLL | CTCTCAACATC | ICKSGTSVKIEC | TTCA |
| | CACAACTCTCCCCAGAGAAG | LLLLLGPGSGL | CGAGCAGGGT | RSLDFQATTMF | GGCC |
| | GTGGTGTGAGGCCATCACGG | GAVVSQHPSRV | TATCTGTAAGA | WYRQFPKKSL | ACAA |
| | AAGATGCTGCTGCTTCTGCT | ICKSGTSVKIEC | GTGGAACCTCT | MLMATSNEGS | CT |
| | GCTTCTGGGGCCAGGCTCCG | RSLDFQATTMF | GTGAAGATCG | KATYEQGVEK | (SEQ |
| | GGCTTGGTGCTGTCGTCTCTC | WYRQFPKKSL | AGTGCCGTTCC | DKFLINHASLTL | ID NO: |
| | AACATCCGAGCAGGGTTATC | MLMATSNEGS | CTGGACTTTCA | STLTVTSAHPE | 471) |
| | TGTAAGAGTGGAACCTCTGT | KATYEQGVEK | GGCCACAACT | DSSFYICSAGGF | |
| | GAAGATCGAGTGCCGTTCCC | DKFLINHASLTL | ATGTTTTGGTA | SGNTIYFGEGS | |
| | TGGACTTTCAGGCCACAACT | STLTVTSAHPE | TCGTCAGTTCC | WLTVV | |
| | ATGTTTTGGTATCGTCAGTTC | DSSFYICSAGGF | CGAAAAAGAG | (SEQ ID NO: | |
| | CCGAAAAAGAGTCTCATGCT | SGNTIYFGEGS | TCTCATGCTGA | 680) | |
| | GATGGCAACTTCCAATGAGG | WLTVVEDLNK | TGGCAACTTCC | | |
| | GCTCCAAGGCCACATACGAG | VFPPEVAVFEP | AATGAGGGCT | | |
| | CAAGGCGTCGAGAAGGACA | SEAEISHTQKA | CCAAGGCCAC | | |
| | AGTTTCTCATCAACCATGCA | TLVCLATGFYP | ATACGAGCAA | | |
| | AGCCTGACCTTGTCCACTCT | DHVELSWWVN | GGCGTCGAGA | | |
| | GACAGTGACCAGTGCCCATC | GKEVHSGVST | AGGACAAGTTT | | |
| | CTGAAGACAGCAGCTTCTAC | DPQPLKEQPA | CTCATCAACCA | | |
| | ATCTGCAGTGCTGGGGGTTT | LNDSRYCLSSR | TGCAAGCCTGA | | |
| | CTCTGGAAACACCATATATT | LRVSATFWQN | CCTTGTCCACT | | |
| | TTGGAGAGGGAAGTTGGCTC | PRNHERCQVQ | CTGACAGTGAC | | |
| | ACTGTTGTAGAGGACCTGAA | FYGLSENDEW | CAGTGCCCATC | | |
| | CAAGGTGTTCCCACCCGAG | TQDRAKPVTQI | CTGAAGACAG | | |
| | GTCGCTGTGTTTGAGCCAT | VSAEAWGRAD | CAGCTTCTACA | | |
| | CAGAAGCAGAGATCTCCCA | CGFTSVSYQQ | TCTGCAGTGCT | | |
| | CACCCAAAAGGCCACACTG | GVLSATILYEI | GGGGGTTTCTC | | |
| | GTGTGCCTGGCCACAGGCT | LLGKATLYAV | TGGAAACACC | | |
| | TCTACCCCGACCACGTGGA | LVSALVLMAM | ATATATTTTGG | | |
| | GCTGAGCTGGTGGGTGAAT | VKRKDF | AGAGGGAAGT | | |
| | GGGAAGGAGGTGCACAGT | (SEQ ID NO: | TGGCTCACTGT | | |
| | GGGGTCAGCACAGACCCG | 678) | TGTAG | | |
| | CAGCCCCTCAAGGAGCAGC | | (SEQ ID NO: | | |
| | CCGCCCTCAATGACTCCAG | | 679) | | |
| | ATACTGCCTGAGCAGCCGC | | | | |
| | CTGAGGGTCTCGGCCACCT | | | | |
| | TCTGGCAGAACCCCCGCAA | | | | |
| | CCACTTCCGCTGTCAAGTC | | | | |
| | CAGTTCTACGGGCTCTCGG | | | | |
| | AGAATGACGAGTGGACCCA | | | | |
| | GGATAGGGCCAAACCCGTC | | | | |
| | ACCCAGATCGTCAGCGCCG | | | | |
| | AGGCCTGGGGTAGAGCAG | | | | |
| | ACTGTGGCTTTACCTCGGT | | | | |
| | GTCCTACCAGCAAGGGGTC | | | | |
| | CTGTCTGCCACCATCCTCT | | | | |
| | ATGAGATCCTGCTAGGGAA | | | | |
| | GGCCACCCTGTATGCTGTG | | | | |
| | CTGGTCAGCGCCCTTGTGT | | | | |
| | TGATGGCCATGGTCAAGAG | | | | |
| | AAAGGATTTCTGA (SEQ ID | | | | |
| | NO: 470) | | | | |

| | CDR1 | CDR2 | CDR2 | CDR3 | CDR3 |
|---|---|---|---|---|---|
| ID | (AA) | (NT) | (AA) | (NT) | (AA) |
| 1 | LGHN | TTCC | FRNR | GCTA | ASGL |
| | T | GCAA | AP | GTGG | ALTE |
| | (SEQ | CCGG | (SEQ | TTTG | GGWY |
| | ID NO: | GCTC | ID NO: | GCCC | EQY |
| | 241) | CT | 243) | TCAC | (SEQ |
| | | (SEQ | | TGAG | ID NO: |
| | | ID NO: | | GGGG | 245) |
| | | 242) | | GCTG | |
| | | | | GTAC | |
| | | | | GAGC | |
| | | | | AGTA | |
| | | | | C | |
| | | | | (SEQ | |
| | | | | ID NO: | |
| | | | | 244) | |
| 2 | SNHL | TTTTA | FYNN | GCCA | ASLR |
| | Y | TAAT | EI | GCCT | WDGD |
| | (SEQ | AATG | (SEQ | CCGA | NEQF |
| | ID NO: | AAAT | ID NO: | TGGG | (SEQ |
| | 248) | C | 250) | ACGG | ID NO: |
| | | (SEQ | | GGAC | 252) |
| | | ID NO: | | AATG | |
| | | 249) | | AGCA | |
| | | | | GTTC | |
| | | | | (SEQ | |
| | | | | ID NO: | |
| | | | | 251) | |
| 3 | MNHN | TCAG | SASEG | GCCA | ASRD |
| | S | CTTCT | T | GCAG | RALN |
| | (SEQ | GAGG | (SEQ | GGAC | TEAF |
| | ID NO: | GTAC | ID NO: | AGGG | (SEQ |
| | 255) | C | 257) | CCCT | ID NO: |
| | | (SEQ | | GAAC | 259) |
| | | ID NO: | | ACTG | |
| | | 256) | | AAGC | |
| | | | | TTTC | |
| | | | | SEQ | |
| | | | | ID NO: | |
| | | | | 258) | |
| 4 | MNHE | TCAG | SVGA | GCCA | ASRN |
| | Y | TTGG | GI | GCAG | GGTLI |
| | (SEQ | TGCT | (SEQ | AAAC | YEQY |
| | ID NO: | GGTA | ID NO: | GGCG | (SEQ |
| | 262) | TC | 264) | GGAC | ID NO: |
| | | (SEQ | | ACTA | 266) |
| | | ID NO: | | ATCT | |
| | | 263) | | ACGA | |
| | | | | GCAG | |
| | | | | TAC | |
| | | | | (SEQ | |
| | | | | ID NO: | |
| | | | | 265) | |
| 5 | SGHRS | TACTT | YFSET | GCCA | ASRQ |
| | (SEQ | CAGT | Q | GCAG | RTELE |
| | ID NO: | GAGA | (SEQ | GCAG | AF |
| | 269) | CACA | ID NO: | CGGA | SEQ |
| | | G | 271) | CAGA | ID NO: |
| | | (SEQ | | ACTT | 273) |
| | | ID NO: | | GAAG | |
| | | 270) | | CTTTC | |
| | | | | (SEQ | |
| | | | | ID NO: | |
| | | | | 272) | |
| 6 | SNHL | TTTTA | FYNN | GCCA | ASSEA |
| | Y | TAAT | EI | GCAG | PDRG |
| | (SEQ | AATG | (SEQ | TGAA | TYEQ |
| | ID NO: | AAAT | ID NO: | GCCC | Y |
| | 276) | C | 278) | CTGA | (SEQ |
| | | (SEQ | | CAGG | ID NO: |
| | | ID NO: | | GGTA | 280) |
| | | 277) | | CCTA | |
| | | | | CGAG | |
| | | | | CAGT | |
| | | | | AC | |
| | | | | (SEQ | |
| | | | | ID NO: | |
| | | | | 279) | |
| 7 | WSHS | TCAG | SAAA | GCCA | ASSEL |
| | Y | CAGC | DI | GCAG | GRGF |
| | (SEQ | TGCT | (SEQ | TGAG | YEQY |
| | ID NO: | GATA | ID NO: | TTGG | (SEQ |
| | 283) | TT | 285) | GCAG | ID NO: |
| | | (SEQ | | GGGT | 287) |
| | | ID NO: | | TTCTA | |
| | | 284) | | CGAG | |
| | | | | CAGT | |
| | | | | AC | |
| | | | | (SEQ | |
| | | | | ID NO: | |
| | | | | 286) | |
| 8 | MNHE | TCAG | SVGA | GCCA | ASSHL |
| | Y | TTGG | GI | GCAG | GAGG |
| | (SEQ | TGCT | (SEQ | CCAT | PHEQ |
| | ID NO: | GGTA | ID NO: | CTGG | Y |
| | 290) | TC | 292) | GGGC | (SEQ |
| | | (SEQ | | GGGA | ID NO: |
| | | ID NO: | | GGGC | 294) |
| | | 291) | | CGCA | |
| | | | | CGAG | |
| | | | | CAGT | |
| | | | | AC | |
| | | | | (SEQ | |
| | | | | ID NO: | |
| | | | | 293) | |
| 9 | SGHV | TTCA | FNYE | GCCA | ASSHT |
| | S | ATTA | AQ | GCAG | DGSY |
| | (SEQ | TGAA | (SEQ | CCAT | EQY |
| | ID NO: | GCCC | ID NO: | ACTG | (SEQ |
| | 297) | AA | 299) | ACGG | ID NO: |
| | | (SEQ | | CTCCT | 301) |
| | | ID NO: | | ACGA | |
| | | 298) | | GCAG | |
| | | | | TAC | |
| | | | | (SEQ | |
| | | | | ID NO: | |
| | | | | 300) | |
| 10 | LNHD | TCAC | SQIVN | GCCA | ASSIG |
| | A | AGAT | D | GTAG | AFAG |
| | (SEQ | AGTA | (SEQ | CATA | QPQH |
| | ID NO: | AATG | ID NO: | GGGG | (SEQ |
| | 304) | AC | 306) | CATTT | ID NO: |
| | | (SEQ | | GCTG | 308) |
| | | ID NO: | | GTCA | |
| | | 305) | | GCCC | |
| | | | | CAGC | |
| | | | | AT | |
| | | | | (SEQ | |
| | | | | ID NO: | |
| | | | | 307) | |
| 11 | SEHN | TTCC | FQNE | GCCA | ASSLA |
| | R | AGAA | AQ | GCAG | PTPGP |
| | (SEQ | TGAA | (SEQ | CTTA | DTGE |
| | ID NO: | GCTC | ID NO: | GCGC | LF |
| | 311) | AA | 313) | CTAC | (SEQ |
| | | (SEQ | | CCCC | ID NO: |
| | | ID NO: | | GGGC | 315) |
| | | 312) | | CGGA | |
| | | | | CACC | |
| | | | | GGGG | |
| | | | | AGCT | |
| | | | | GTTT | |
| | | | | (SEQ | |
| | | | | ID NO: | |
| | | | | 314) | |
| 12 | MNHE | TCAA | SMNV | GCCA | ASSLG |
| | Y | TGAA | EV | GCAG | NIYEQ |
| | (SEQ | TGTT | (SEQ | TTTA | Y |
| | ID NO: | GAGG | ID NO: | GGGA | SEQ |
| | 318) | TG | 320) | ATAT | ID NO: |
| | | (SEQ | | CTAC | 322) |
| | | ID NO: | | GAGC | |
| | | 319) | | AGTA | |
| | | | | C | |
| | | | | (SEQ | |
| | | | | ID NO: | |
| | | | | 321) | |
| 13 | SGHT | TTCC | FQGT | GCCA | ASSLG |
| | A | AAGG | GA | GCAG | SGEQF |
| | (SEQ | CACG | (SEQ | CTTA | (SEQ |
| | ID NO: | GGTG | ID NO: | GGGA | ID NO: |
| | 325) | CG | 327) | GCGG | 329) |
| | | (SEQ | | TGAG | |
| | | ID NO: | | CAGT | |
| | | 326) | | TC | |
| | | | | (SEQ | |
| | | | | ID NO: | |
| | | | | 328) | |
| 14 | SGHTS | TATG | YDEG | GCCA | ASSLQ |
| | (SEQ | ACGA | EE | GCAG | TSYEQ |
| | ID NO: | GGGT | (SEQ | CTTA | Y |
| | 332) | GAAG | ID NO: | CAGA | (SEQ |
| | | AG | 334) | CATC | ID NO: |
| | | (SEQ | | CTAC | 336) |
| | | ID NO: | | GAGC | |
| | | 333) | | AGTA | |
| | | | | C | |
| | | | | (SEQ | |
| | | | | ID NO: | |
| | | | | 335) | |
| 15 | MNHE | TCAA | SMNV | GCCA | ASSLS |
| | Y | TGAA | EV | GCAG | WGTG |
| | (SEQ | TGTT | (SEQ | TTTAT | KRAD |
| | ID NO: | GAGG | ID NO: | CGTG | TQY |
| | 339) | TG | 341) | GGGG | SEQ |
| | | (SEQ | | ACCG | ID NO: |
| | | ID NO: | | GCAA | 343) |
| | | 340) | | AAGA | |
| | | | | GCAG | |
| | | | | ATAC | |
| | | | | GCAG | |
| | | | | TAT | |
| | | | | (SEQ | |
| | | | | ID NO: | |
| | | | | 342) | |
| 16 | SEHN | TTCC | FQNE | GCCA | ASSLV |
| | R | AGAA | AQ | GCAG | GGGS |
| | (SEQ | TGAA | (SEQ | CTTA | NEQF |
| | ID NO: | GCTC | ID NO: | GTTG | (SEQ |
| | 346) | AA | 348) | GCGG | ID NO: |
| | | (SEQ | | CGGG | 350) |
| | | ID NO: | | AGCA | |
| | | 347) | | ATGA | |
| | | | | GCAG | |
| | | | | TTC | |
| | | | | (SEQ | |
| | | | | ID NO: | |
| | | | | 349) | |
| 17 | SEHN | TTCC | FQNE | GCCA | ASSLV |
| | R | AGAA | AQ | GCAG | GGTE |
| | (SEQ | TGAA | (SEQ | CTTA | AF |
| | ID NO: | GCTC | ID NO: | GTCG | (SEQ |
| | 353) | AA | 355) | GAGG | ID NO: |
| | | (SEQ | | CACT | 357) |
| | | ID NO: | | GAAG | |
| | | 354) | | CTTTC | |
| | | | | (SEQ | |
| | | | | ID NO: | |
| | | | | 356) | |
| 18 | SEHN | TTCC | FQNE | GCCA | ASSPD |
| | R | AGAA | AQ | GCAG | RNLG |
| | (SEQ | TGAA | (SEQ | CCCC | QY |
| | ID NO: | GCTC | ID NO: | GATC | (SEQ |
| | 360) | AA | 362) | GGAA | ID NO: |
| | | (SEQ | | TCTC | 364) |
| | | ID NO: | | GGGC | |
| | | 361) | | AGTA | |
| | | | | C | |
| | | | | (SEQ | |
| | | | | ID NO: | |
| | | | | 363) | |
| 19 | SGHD | TTTGT | FVKES | GCCA | ASSQ |
| | N | GAAA | K | GCAG | VQAF |
| | (SEQ | GAGT | (SEQ | CCAA | NEQF |
| | ID NO: | CTAA | ID NO: | GTCC | (SEQ |
| | 367) | A | 369) | AGGC | ID NO: |
| | | (SEQ | | TTTTA | 371) |
| | | ID NO: | | ATGA | |
| | | 368) | | GCAG | |
| | | | | TTC | |
| | | | | (SEQ | |
| | | | | ID NO: | |
| | | | | 370) | |
| 20 | SGHA | TTTCA | FQNN | GCCA | ASSRG |
| | T | GAAT | GV | GCAG | EPGSG |
| | (SEQ | AACG | (SEQ | TCGA | ANVL |
| | ID NO: | GTGT | ID NO: | GGGG | T |
| | 374) | A | 376) | AGCC | (SEQ |
| | | (SEQ | | AGGC | ID NO: |
| | | ID NO: | | TCTG | 378) |
| | | 375) | | GGGC | |
| | | | | CAAC | |
| | | | | GTCC | |
| | | | | TGAC | |
| | | | | T | |
| | | | | (SEQ | |
| | | | | ID NO: | |
| | | | | 377) | |
| 21 | SGHD | TATT | YYEE | GCCA | ASSSG |
| | T | ATGA | EE | GCAG | TDPSG |
| | (SEQ | GGAG | (SEQ | CTCC | ANVL |
| | ID NO: | GAAG | ID NO: | GGGA | T |
| | 381) | AG | 383) | CAGA | (SEQ |
| | | (SEQ | | CCCC | ID NO: |
| | | ID NO: | | TCTG | 385) |
| | | 382) | | GGGC | |
| | | | | CAAC | |
| | | | | GTCC | |
| | | | | TGAC | |
| | | | | T | |
| | | | | (SEQ | |
| | | | | ID NO: | |
| | | | | 384) | |
| 22 | MNHE | TCAA | SMNV | GCCA | ASSTG |
| | Y | TGAA | EV | GCAG | QNIGG |
| | (SEQ | TGTT | (SEQ | TACC | ELF |
| | ID NO: | GAGG | ID NO: | GGAC | (SEQ |
| | 388) | TG | 390) | AGAA | ID NO: |
| | | (SEQ | | TATA | 392) |
| | | ID NO: | | GGCG | |
| | | 389) | | GGGA | |
| | | | | GCTG | |
| | | | | TTT | |
| | | | | (SEQ | |
| | | | | ID NO: | |
| | | | | 391) | |
| 23 | SEHN | TTCC | FQNE | GCCA | ASSTH |
| | R | AGAA | AQ | GCAG | EKTG |
| | (SEQ | TGAA | (SEQ | CACA | WKSP |
| | ID NO: | GCTC | ID NO: | CATG | LH |
| | 395) | AA | 397) | AAAA | (SEQ |
| | | (SEQ | | GACA | ID NO: |
| | | ID NO: | | GGGT | 399) |
| | | 396) | | GGAA | |
| | | | | ATCA | |
| | | | | CCCC | |
| | | | | TCCA | |
| | | | | C | |
| | | | | (SEQ | |
| | | | | ID NO: | |
| | | | | 398) | |
| 24 | SNHL | TTTTA | FYNN | GCCA | ASSTH |
| | Y | TAAT | EI | GCAG | SDRN |
| | (SEQ | AATG | (SEQ | TACG | LNTE |
| | ID NO: | AAAT | ID NO: | CACT | AF |
| | 402) | C | 404) | CTGA | (SEQ |
| | | (SEQ | | CAGG | ID NO: |
| | | ID NO: | | AACT | 406) |
| | | 403) | | TGAA | |
| | | | | CACT | |
| | | | | GAAG | |
| | | | | CTTTC | |
| | | | | (SEQ | |
| | | | | ID NO: | |
| | | | | 405) | |
| 25 | SNHL | TTTTA | FYNN | GCCA | ASSV |
| | Y | TAAT | EI | GCAG | QATG |
| | (SEQ | AATG | (SEQ | CGTA | HGYT |
| | ID NO: | AAAT | ID NO: | CAGG | (SEQ |
| | 409) | C | 411) | CTAC | ID NO: |
| | | (SEQ | | GGGC | 413) |
| | | ID NO: | | CATG | |
| | | 410) | | GCTA | |
| | | | | CACC | |
| | | | | (SEQ | |
| | | | | ID NO: | |
| | | | | 412) | |
| 26 | SGHV | TTCC | FQNE | GCCA | ASTPS |
| | S | AGAA | AQ | GCAC | GYNS |
| | (SEQ | TGAA | (SEQ | CCCC | WEQF |
| | ID NO: | GCTC | ID NO: | TCTG | (SEQ |
| | 416) | AA | 418) | GCTA | ID NO: |
| | | (SEQ | | TAAC | 420) |
| | | ID NO: | | TCTTG | |
| | | 417) | | GGAG | |
| | | | | CAGT | |
| | | | | TC | |
| | | | | (SEQ | |
| | | | | ID NO: | |
| | | | | 419) | |
| 27 | MNHE | TCAA | SMNV | GCCA | ATAP |
| | Y | TGAA | EV | CCGC | RGSN |
| | (SEQ | TGTT | (SEQ | CCCC | QPQH |
| | ID NO: | GAGG | ID NO: | AGGG | (SEQ |
| | 423) | TG | 425) | GTAG | ID NO: |
| | | (SEQ | | CAAT | 427) |
| | | ID NO: | | CAGC | |
| | | 424) | | CCCA | |
| | | | | GCAT | |
| | | | | (SEQ | |
| | | | | ID NO: | |
| | | | | 426) | |
| 28 | DFQA | TCCA | SNEGS | AGGG | RAWG |
| | TT | ATGA | KA | CCTG | GSSY |
| | (SEQ | GGGC | (SEQ | GGGC | NEQF |
| | ID NO: | TCCA | ID NO: | GGGA | (SEQ |
| | 430) | AGGC | 432) | GCTC | ID NO: |
| | | C | | CTAC | 434) |
| | | (SEQ | | AATG | |
| | | ID NO: | | AGCA | |
| | | 431) | | GTTC | |
| | | | | (SEQ | |
| | | | | ID NO: | |
| | | | | 433) | |
| 29 | DFQA | TCCA | SNEGS | AGTG | SARIG |
| | TT | ATGA | KA | CTAG | QGFM |
| | (SEQ | GGGC | (SEQ | AATT | NEQF |
| | ID NO: | TCCA | ID NO: | GGAC | (SEQ |
| | 437) | AGGC | 439) | AGGG | ID NO: |
| | | C | | TTTTA | 441) |
| | | (SEQ | | TGAA | |
| | | ID NO: | | TGAG | |
| | | 438) | | CAGT | |
| | | | | TC | |
| | | | | (SEQ | |
| | | | | ID NO: | |
| | | | | 440) | |
| 30 | DFQA | TCCA | SNEGS | AGTG | SAPG |
| | TT | ATGA | KA | CTCCT | WRGT |
| | (SEQ | GGGC | (SEQ | GGGT | EAF |
| | ID NO: | TCCA | ID NO: | GGCG | (SEQ |
| | 444) | AGGC | 446) | TGGC | ID NO: |
| | | C | | ACTG | 448) |
| | | (SEQ | | AAGC | |
| | | ID NO: | | TTTC | |
| | | 445) | | (SEQ | |
| | | | | ID NO: | |
| | | | | 447) | |
| 31 | LGHD | TACA | YNNK | GCCA | ASSQ |
| | T | ATAA | EL | GCAG | DGLE |
| | (SEQ | TAAG | (SEQ | CCAA | QY |
| | ID NO: | GAGC | ID NO: | GATG | (SEQ |
| | 451) | TC | 453) | GACT | ID NO: |
| | | (SEQ | | TGAG | 455) |
| | | ID NO: | | CAGT | |
| | | 452) | | AC | |
| | | | | (SEQ | |
| | | | | ID NO: | |
| | | | | 454) | |
| 32 | LGHD | TACA | YNNK | GCCA | ASSQE |
| | T | ATAA | EL | GCAG | GSQD |
| | (SEQ | TAAG | (SEQ | CCAA | HTSG |
| | ID NO: | GAGC | ID NO: | GAAG | RATE |
| | 458) | TC | 460) | GATC | QY |
| | | (SEQ | | TCAG | (SEQ |
| | | ID NO | | GACC | ID NO: |
| | | 459) | | ACAC | 462) |
| | | | | TAGC | |
| | | | | GGGA | |
| | | | | GGGC | |
| | | | | CACC | |
| | | | | GAGC | |
| | | | | AGTA | |
| | | | | C | |
| | | | | (SEQ | |
| | | | | ID NO: | |
| | | | | 461) | |
| 33 | MNHE | TCAG | SVGA | GCCA | ASSLT |
| | Y | TTGG | GI | GCAG | EGRSE |
| | (SEQ | TGCT | (SEQ | CCTG | QY |
| | ID NO: | GGTA | ID NO: | ACAG | (SEQ |
| | 465) | TC | 467) | AAGG | ID NO: |
| | | (SEQ | | AAGA | 469) |
| | | ID NO: | | AGCG | |
| | | 466) | | AGCA | |
| | | | | GTAC | |
| | | | | (SEQ | |
| | | | | ID NO: | |
| | | | | 468) | |
| 34 | DFQA | TCCA | SNEGS | AGTG | SAGG |
| | TT | ATGA | KA | CTGG | FSGNT |
| | (SEQ | GGGC | (SEQ | GGGT | IY |
| | ID NO: | TCCA | ID NO: | TTCTC | (SEQ |
| | 472) | AGGC | 474) | TGGA | ID NO: |
| | | C | | AACA | 476) |
| | | (SEQ | | CCAT | |
| | | ID NO: | | ATAT | |
| | | 473) | | (SEQ | |
| | | | | ID NO: | |
| | | | | 475) | |

### Example 3. Lentivirus Design and Transduction

### Vector Design

Unique complementary determining regions (CDR) from selected TCR α/β pairs will be combined with the associated constant regions to form a full length TCR DNA sequence. Sequences will have TCRα with the stop codon removed followed by a furin F2A cleavage site and then a TCRβ containing a stop codon. Native leader sequences will be used for each TCR sequence. The DNA will be synthesized and cloned into an EF1a promotor vector with puromycin resistance to produce lenti-virus constructs (VectorBuilder™)

### Lentiviral Transduction of TCR KO Jurkat Cell Lines

Day 1: TCR KO Jurkat cells will be removed from flasks and counted. The cells will be washed by centrifuging at 1500 rpm for five minutes and resuspended in R10 (R10 Media: Gibco™, 61870 supplemented with 10% FBS Gibco™, 16140 and 1× Sodium Pyruvate Gibco™, 11360) at a density of 1.5e6 cells/mL. 500 μL of cells will be seeded (for a density of 7.5e5 cells/well) to appropriate wells of a 12 well plate(s) according to plate layout. In a BSL2+ environment, lentiviral constructs will be diluted in R10 media with 2× (final concentration: 4 μg/mL) Polybrene (Millipore™, Cat #: TR-1003-G) to concentrations of 20 Multiplicity of Infection (MOI) per 500 μL. 500 μL of each construct will be added to appropriate wells according to plate layout. Plate(s) will be centrifuged at 2000 rpm for two hours at 30° C. After centrifuging 1 mL R10 will be added to each well. Plates will be incubated at 37° C./5% CO2 overnight.

Day 2: Cells will be transferred to individual 15 mL tubes and washed by centrifuging at 300×g for five minutes. Pellets will be resuspended in DPBS (Gibco™ 14140). Washes will be repeated for a total of three washes. After the final wash, cells will be resuspended in 1 mL R10. Cells will be transferred to a 6 well plate and add 1 mL of prepared R10 media containing 2× (final concentration 1.25 μg/mL) puromycin (Gibco™ A11138-03). Cells will be returned to the incubator at 37° C./5% CO₂.

Day 5: Cells will be counted, and density will be scaled to be 2e5 cells/mL in an appropriately sized plate/flask for the volume. For healthy cells, puromycin concentration may be increased from 1.25 μg/mL to 2.5 μg/mL. Cells will be returned to the incubator at 37° C./5% CO₂.

Day 8 (and every 3-4 days after): Cells will be counted and scaled to maintain a density of 2e5 cells/mL. Puromycin will be gradually increased up to a final concentration of 5 μg/mL.

### Example 4. TCR Engagement Assay

A TCR engagement assay will be performed to determine if the TCRα/β pair is able to bind to a defined peptide-MHC (pMHC) complex and induce a T-cell response. Antigen presenting cells (APC) will be harvested and plated into white clear bottom 96 well plates at 3e5 cells/well in 40 μL in RPMI 1640+10% FBS). Peptides will be prepared by adding Dimethylsulfoxide (DMSO) or water to lyophilized powder to achieve a 10 mg/mL concentration. Peptides will be exogenously loaded onto APCs by diluting to a 10× final concentration in media and adding 10 μL to the wells. DMSO or water and/or wild type (wt) peptide at the same concentrations will be used as controls. The cells will be placed in a 37° C., 5% CO2 incubator for 2 hours. Meanwhile, reporter T-cells expressing a single TCR a/b pair from Table 3 will be harvested and diluted to 3e6 cells/mL. After the 2 hour incubation, 50 μL of T-cells will be added to each well, resulting in a 2:1 APC:T-cell ratio. Cells will be returned to the incubator for 5-6 hours. After the incubation, 100 μL of BioGlo™ luciferase reagent (Promega™, cat #G7940) will be added to each well. Plates will be placed on a rotary plate shaker at medium speed for 2 minutes. Plates will be read on an Envision™ (Perkin Elmer™) plate reader using a 0.5 sec luminescence detection. Cross-talk corrected values will be analyzed using GraphPad™ Prism™ graphing software.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

The following list of embodiments is intended to complement, rather than displace or supersede, the previous descriptions:

Embodiment 1. A T-cell receptor (TCR) comprising an alpha chain and a beta chain, wherein:
(a) the alpha chain comprises a complementarity determining region 3 (CDR3) comprising the amino acid sequence of SEQ ID NO: 7, and the beta chain comprises a CDR3 comprising the amino acid sequence of SEQ ID NO: 245;
(b) the alpha chain comprises a CDR3 comprising the amino acid sequence of SEQ ID NO: 14, and the beta chain comprises a CDR3 comprising the amino acid sequence of SEQ ID NO: 252;
(c) the alpha chain comprises a CDR3 comprising the amino acid sequence of SEQ ID NO: 21, and the beta chain comprises a CDR3 comprising the amino acid sequence of SEQ ID NO: 259;
(d) the alpha chain comprises a CDR3 comprising the amino acid sequence of SEQ ID NO: 28, and the beta chain comprises a CDR3 comprising the amino acid sequence of SEQ ID NO: 266;
(e) the alpha chain comprises a CDR3 comprising the amino acid sequence of SEQ ID NO: 35, and the beta chain comprises a CDR3 comprising the amino acid sequence of SEQ ID NO: 273;
(f) the alpha chain comprises a CDR3 comprising the amino acid sequence of SEQ ID NO: 42, and the beta chain comprises a CDR3 comprising the amino acid sequence of SEQ ID NO: 280;
(g) the alpha chain comprises a CDR3 comprising the amino acid sequence of SEQ ID NO: 49, and the beta chain comprises a CDR3 comprising the amino acid sequence of SEQ ID NO: 287;
(h) the alpha chain comprises a CDR3 comprising the amino acid sequence of SEQ ID NO: 56, and the beta chain comprises a CDR3 comprising the amino acid sequence of SEQ ID NO: 294;
(i) the alpha chain comprises a CDR3 comprising the amino acid sequence of SEQ ID NO: 63, and the beta chain comprises a CDR3 comprising the amino acid sequence of SEQ ID NO: 301;
(j) the alpha chain comprises a CDR3 comprising the amino acid sequence of SEQ ID NO: 70, and the beta chain comprises a CDR3 comprising the amino acid sequence of SEQ ID NO: 308;
(k) the alpha chain comprises a CDR3 comprising the amino acid sequence of SEQ ID NO: 77, and the beta chain comprises a CDR3 comprising the amino acid sequence of SEQ ID NO: 315;
(l) the alpha chain comprises a CDR3 comprising the amino acid sequence of SEQ ID NO: 84, and the beta chain comprises a CDR3 comprising the amino acid sequence of SEQ ID NO: 322;
(m) the alpha chain comprises a CDR3 comprising the amino acid sequence of SEQ ID NO: 91, and the beta chain comprises a CDR3 comprising the amino acid sequence of SEQ ID NO: 329;
(n) the alpha chain comprises a CDR3 comprising the amino acid sequence of SEQ ID NO: 98, and the beta chain comprises a CDR3 comprising the amino acid sequence of SEQ ID NO: 336;
(o) the alpha chain comprises a CDR3 comprising the amino acid sequence of SEQ ID NO: 105, and the beta chain comprises a CDR3 comprising the amino acid sequence of SEQ ID NO: 343;
(p) the alpha chain comprises a CDR3 comprising the amino acid sequence of SEQ ID NO: 112, and the beta chain comprises a CDR3 comprising the amino acid sequence of SEQ ID NO: 350;
(q) the alpha chain comprises a CDR3 comprising the amino acid sequence of SEQ ID NO: 119, and the beta chain comprises a CDR3 comprising the amino acid sequence of SEQ ID NO: 357;
(r) the alpha chain comprises a CDR3 comprising the amino acid sequence of SEQ ID NO: 126, and the beta chain comprises a CDR3 comprising the amino acid sequence of SEQ ID NO: 364;
(s) the alpha chain comprises a CDR3 comprising the amino acid sequence of SEQ ID NO: 133, and the beta chain comprises a CDR3 comprising the amino acid sequence of SEQ ID NO: 371;
(t) the alpha chain comprises a CDR3 comprising the amino acid sequence of SEQ ID NO: 140, and the beta chain comprises a CDR3 comprising the amino acid sequence of SEQ ID NO: 378;
(u) the alpha chain comprises a CDR3 comprising the amino acid sequence of SEQ ID NO: 147, and the beta chain comprises a CDR3 comprising the amino acid sequence of SEQ ID NO: 385;
(v) the alpha chain comprises a CDR3 comprising the amino acid sequence of SEQ ID NO: 154, and the beta chain comprises a CDR3 comprising the amino acid sequence of SEQ ID NO: 392;
(w) the alpha chain comprises a CDR3 comprising the amino acid sequence of SEQ ID NO: 161, and the beta chain comprises a CDR3 comprising the amino acid sequence of SEQ ID NO: 399;
(x) the alpha chain comprises a CDR3 comprising the amino acid sequence of SEQ ID NO: 168, and the beta chain comprises a CDR3 comprising the amino acid sequence of SEQ ID NO: 406;
(y) the alpha chain comprises a CDR3 comprising the amino acid sequence of SEQ ID NO: 175, and the beta chain comprises a CDR3 comprising the amino acid sequence of SEQ ID NO: 413;
(z) the alpha chain comprises a CDR3 comprising the amino acid sequence of SEQ ID NO: 182, and the beta chain comprises a CDR3 comprising the amino acid sequence of SEQ ID NO: 420;
(aa) the alpha chain comprises a CDR3 comprising the amino acid sequence of SEQ ID NO: 189, and the beta chain comprises a CDR3 comprising the amino acid sequence of SEQ ID NO: 427;
(bb) the alpha chain comprises a CDR3 comprising the amino acid sequence of SEQ ID NO: 196, and the beta chain comprises a CDR3 comprising the amino acid sequence of SEQ ID NO: 434;
(cc) the alpha chain comprises a CDR3 comprising the amino acid sequence of SEQ ID NO: 203, and the beta chain comprises a CDR3 comprising the amino acid sequence of SEQ ID NO: 441;
(dd) the alpha chain comprises a CDR3 comprising the amino acid sequence of SEQ ID NO: 210, and the beta chain comprises a CDR3 comprising the amino acid sequence of SEQ ID NO: 448;
(ee) the alpha chain comprises a CDR3 comprising the amino acid sequence of SEQ ID NO: 217, and the beta chain comprises a CDR3 comprising the amino acid sequence of SEQ ID NO: 455;
(ff) the alpha chain comprises a CDR3 comprising the amino acid sequence of SEQ ID NO: 224, and the beta chain comprises a CDR3 comprising the amino acid sequence of SEQ ID NO: 462;
(gg) the alpha chain comprises a CDR3 comprising the amino acid sequence of SEQ ID NO: 231, and the beta chain comprises a CDR3 comprising the amino acid sequence of SEQ ID NO: 469; or
(hh) the alpha chain comprises a CDR3 comprising the amino acid sequence of SEQ ID NO: 238, and the beta chain comprises a CDR3 comprising the amino acid sequence of SEQ ID NO: 476.

Embodiment 2. A T-cell receptor (TCR) comprising an alpha chain and a beta chain, wherein:
(a) the alpha chain comprises a complementarity determining region 1 (CDR1) comprising the amino acid sequence of SEQ ID NO: 3, a complementarity determining region 2 (CDR2) comprising the amino acid sequence of SEQ ID NO: 5, and a complementarity determining region 3 (CDR3) comprising the amino acid sequence of SEQ ID NO: 7, and the beta chain comprises a CDR1 comprising the amino acid sequence of SEQ ID NO: 241, a CDR2 comprising the amino acid sequence of SEQ ID NO: 243, and a CDR3 comprising the amino acid sequence of SEQ ID NO: 245;
(b) the alpha chain comprises a CDR1 comprising the amino acid sequence of SEQ ID NO: 10, a CDR2 comprising the amino acid sequence of SEQ ID NO: 12, and a CDR3 comprising the amino acid sequence of SEQ ID NO: 14, and the beta chain comprises a CDR1 comprising the amino acid sequence of SEQ ID NO: 248, a CDR2 comprising the amino acid sequence of SEQ ID NO: 250, and a CDR3 comprising the amino acid sequence of SEQ ID NO: 252;
(c) the alpha chain comprises a CDR1 comprising the amino acid sequence of SEQ ID NO: 17, a CDR2 comprising the amino acid sequence of SEQ ID NO: 19, and a CDR3 comprising the amino acid sequence of SEQ ID NO: 21, and the beta chain comprises a CDR1 comprising the amino acid sequence of SEQ ID NO: 255, a CDR2 comprising the amino acid sequence of SEQ ID NO: 257, and a CDR3 comprising the amino acid sequence of SEQ ID NO: 259;
(d) the alpha chain comprises a CDR1 comprising the amino acid sequence of SEQ ID NO: 24, a CDR2 comprising the amino acid sequence of SEQ ID NO: 26, and a CDR3 comprising the amino acid sequence of SEQ ID NO: 28, and the beta chain comprises a CDR1 comprising the amino acid sequence of SEQ ID NO: 262, a CDR2 comprising the amino acid sequence of SEQ ID NO: 264, and a CDR3 comprising the amino acid sequence of SEQ ID NO: 266;
(e) the alpha chain comprises a CDR1 comprising the amino acid sequence of SEQ ID NO: 31, a CDR2 comprising the amino acid sequence of SEQ ID NO: 33, and a CDR3 comprising the amino acid sequence of SEQ ID NO: 35, and the beta chain comprises a CDR1 comprising the amino acid sequence of SEQ ID NO: 269, a CDR2 comprising the amino acid sequence of SEQ ID NO: 271, and a CDR3 comprising the amino acid sequence of SEQ ID NO: 273;
(f) the alpha chain comprises a CDR1 comprising the amino acid sequence of SEQ ID NO: 38, a CDR2 comprising the amino acid sequence of SEQ ID NO: 40, and a CDR3 comprising the amino acid sequence of SEQ ID NO: 42, and the beta chain comprises a CDR1 comprising the amino acid sequence of SEQ ID NO: 276, a CDR2 comprising the amino acid sequence of SEQ ID NO: 278, and a CDR3 comprising the amino acid sequence of SEQ ID NO: 280;
(g) the alpha chain comprises a CDR1 comprising the amino acid sequence of SEQ ID NO: 45, a CDR2 comprising the amino acid sequence of SEQ ID NO: 47, and a CDR3 comprising the amino acid sequence of SEQ ID NO: 49, and the beta chain comprises a CDR1 comprising the amino acid sequence of SEQ ID NO: 283, a CDR2 comprising the amino acid sequence of SEQ ID NO: 285, and a CDR3 comprising the amino acid sequence of SEQ ID NO: 287;
(h) the alpha chain comprises a CDR1 comprising the amino acid sequence of SEQ ID NO: 52, a CDR2 comprising the amino acid sequence of SEQ ID NO: 54, and a CDR3 comprising the amino acid sequence of SEQ ID NO: 56, and the beta chain comprises a CDR1 comprising the amino acid sequence of SEQ ID NO: 290, a CDR2 comprising the amino acid sequence of SEQ ID NO: 292, and a CDR3 comprising the amino acid sequence of SEQ ID NO: 294;
(i) the alpha chain comprises a CDR1 comprising the amino acid sequence of SEQ ID NO: 59, a CDR2 comprising the amino acid sequence of SEQ ID NO: 61, and a CDR3 comprising the amino acid sequence of SEQ ID NO: 63, and the beta chain comprises a CDR1 comprising the amino acid sequence of SEQ ID NO: 297, a CDR2 comprising the amino acid sequence of SEQ ID NO: 299, and a CDR3 comprising the amino acid sequence of SEQ ID NO: 301;
(j) the alpha chain comprises a CDR1 comprising the amino acid sequence of SEQ ID NO: 66, a CDR2 comprising the amino acid sequence of SEQ ID NO: 68, and a CDR3 comprising the amino acid sequence of SEQ ID NO: 70, and the beta chain comprises a CDR1 comprising the amino acid sequence of SEQ ID NO: 304, a CDR2 comprising the amino acid sequence of SEQ ID NO: 306, and a CDR3 comprising the amino acid sequence of SEQ ID NO: 308;
(k) the alpha chain comprises a CDR1 comprising the amino acid sequence of SEQ ID NO: 73, a CDR2 comprising the amino acid sequence of SEQ ID NO: 75, and a CDR3 comprising the amino acid sequence of SEQ ID NO: 77, and the beta chain comprises a CDR1 comprising the amino acid sequence of SEQ ID NO: 311, a CDR2 comprising the amino acid sequence of SEQ ID NO: 313, and a CDR3 comprising the amino acid sequence of SEQ ID NO: 315;
(l) the alpha chain comprises a CDR1 comprising the amino acid sequence of SEQ ID NO: 80, a CDR2 comprising the amino acid sequence of SEQ ID NO: 82, and a CDR3 comprising the amino acid sequence of SEQ ID NO: 84, and the beta chain comprises a CDR1 comprising the amino acid sequence of SEQ ID NO: 318, a CDR2 comprising the amino acid sequence of SEQ ID NO: 320, and a CDR3 comprising the amino acid sequence of SEQ ID NO: 322;
(m) the alpha chain comprises a CDR1 comprising the amino acid sequence of SEQ ID NO: 87, a CDR2 comprising the amino acid sequence of SEQ ID NO: 89, and a CDR3 comprising the amino acid sequence of SEQ ID NO: 91, and the beta chain comprises a CDR1 comprising the amino acid sequence of SEQ ID NO: 325, a CDR2 comprising the amino acid sequence of SEQ ID NO: 327, and a CDR3 comprising the amino acid sequence of SEQ ID NO: 329;
(n) the alpha chain comprises a CDR1 comprising the amino acid sequence of SEQ ID NO: 94, a CDR2 comprising the amino acid sequence of SEQ ID NO: 96, and a CDR3 comprising the amino acid sequence of SEQ ID NO: 98, and the beta chain comprises a CDR1 comprising the amino acid sequence of SEQ ID NO: 332, a CDR2 comprising the amino acid sequence of SEQ ID NO: 334, and a CDR3 comprising the amino acid sequence of SEQ ID NO: 336;
(o) the alpha chain comprises a CDR1 comprising the amino acid sequence of SEQ ID NO: 101, a CDR2 comprising the amino acid sequence of SEQ ID NO: 103, and a CDR3 comprising the amino acid sequence of SEQ ID NO: 105, and the beta chain comprises a CDR1 comprising the amino acid sequence of SEQ ID NO: 339, a CDR2 comprising the amino acid sequence of SEQ ID NO: 341, and a CDR3 comprising the amino acid sequence of SEQ ID NO: 343;
(p) the alpha chain comprises a CDR1 comprising the amino acid sequence of SEQ ID NO: 108, a CDR2 comprising the amino acid sequence of SEQ ID NO: 110, and a CDR3 comprising the amino acid sequence of SEQ ID NO: 112, and the beta chain comprises a CDR1 comprising the amino acid sequence of SEQ ID NO: 346, a CDR2 comprising the amino acid sequence of SEQ ID NO: 348, and a CDR3 comprising the amino acid sequence of SEQ ID NO: 350;
(q) the alpha chain comprises a CDR1 comprising the amino acid sequence of SEQ ID NO: 115, a CDR2 comprising the amino acid sequence of SEQ ID NO: 117, and a CDR3 comprising the amino acid sequence of SEQ ID NO: 119, and the beta chain comprises a CDR1 comprising the amino acid sequence of SEQ ID NO: 353, a CDR2 comprising the amino acid sequence of SEQ ID NO: 355, and a CDR3 comprising the amino acid sequence of SEQ ID NO: 357;
(r) the alpha chain comprises a CDR1 comprising the amino acid sequence of SEQ ID NO: 122, a CDR2 comprising the amino acid sequence of SEQ ID NO: 124, and a CDR3 comprising the amino acid sequence of SEQ ID NO: 126, and the beta chain comprises a CDR1 comprising the amino acid sequence of SEQ ID NO: 360, a CDR2 comprising the amino acid sequence of SEQ ID NO: 362, and a CDR3 comprising the amino acid sequence of SEQ ID NO: 364;
(s) the alpha chain comprises a CDR1 comprising the amino acid sequence of SEQ ID NO: 129, a CDR2 comprising the amino acid sequence of SEQ ID NO: 131, and a CDR3 comprising the amino acid sequence of SEQ ID NO: 133, and the beta chain comprises a CDR1 comprising the amino acid sequence of SEQ ID NO: 367, a CDR2 comprising the amino acid sequence of SEQ ID NO: 369, and a CDR3 comprising the amino acid sequence of SEQ ID NO: 371;
(t) the alpha chain comprises a CDR1 comprising the amino acid sequence of SEQ ID NO: 136, a CDR2 comprising the amino acid sequence of SEQ ID NO: 138, and a CDR3 comprising the amino acid sequence of SEQ ID NO: 140, and the beta chain comprises a CDR1 comprising the amino acid sequence of SEQ ID NO: 374, a CDR2 comprising the amino acid sequence of SEQ ID NO: 376, and a CDR3 comprising the amino acid sequence of SEQ ID NO: 378;
(u) the alpha chain comprises a CDR1 comprising the amino acid sequence of SEQ ID NO: 143, a CDR2 comprising the amino acid sequence of SEQ ID NO: 145, and a CDR3 comprising the amino acid sequence of SEQ ID NO: 147, and the beta chain comprises a CDR1 comprising the amino acid sequence of SEQ ID NO: 381, a CDR2 comprising the amino acid sequence of SEQ ID NO: 383, and a CDR3 comprising the amino acid sequence of SEQ ID NO: 385;
(v) the alpha chain comprises a CDR1 comprising the amino acid sequence of SEQ ID NO: 150, a CDR2 comprising the amino acid sequence of SEQ ID NO: 152, and a CDR3 comprising the amino acid sequence of SEQ ID NO: 154, and the beta chain comprises a CDR1 comprising the amino acid sequence of SEQ ID NO: 388, a CDR2 comprising the amino acid sequence of SEQ ID NO: 390, and a CDR3 comprising the amino acid sequence of SEQ ID NO: 392;
(w) the alpha chain comprises a CDR1 comprising the amino acid sequence of SEQ ID NO: 157, a CDR2 comprising the amino acid sequence of SEQ ID NO: 159, and a CDR3 comprising the amino acid sequence of SEQ ID NO: 161, and the beta chain comprises a CDR1 comprising the amino acid sequence of SEQ ID NO: 395, a CDR2 comprising the amino acid sequence of SEQ ID NO: 397, and a CDR3 comprising the amino acid sequence of SEQ ID NO: 399;
(x) the alpha chain comprises a CDR1 comprising the amino acid sequence of SEQ ID NO: 164, a CDR2 comprising the amino acid sequence of SEQ ID NO: 166, and a CDR3 comprising the amino acid sequence of SEQ ID NO: 168, and the beta chain comprises a CDR1 comprising the amino acid sequence of SEQ ID NO: 402, a CDR2 comprising the amino acid sequence of SEQ ID NO: 404, and a CDR3 comprising the amino acid sequence of SEQ ID NO: 406;
(y) the alpha chain comprises a CDR1 comprising the amino acid sequence of SEQ ID NO: 171, a CDR2 comprising the amino acid sequence of SEQ ID NO: 173, and a CDR3 comprising the amino acid sequence of SEQ ID NO: 175, and the beta chain comprises a CDR1 comprising the amino acid sequence of SEQ ID NO: 409, a CDR2 comprising the amino acid sequence of SEQ ID NO: 411, and a CDR3 comprising the amino acid sequence of SEQ ID NO: 413;
(z) the alpha chain comprises a CDR1 comprising the amino acid sequence of SEQ ID NO: 178, a CDR2 comprising the amino acid sequence of SEQ ID NO: 180, and a CDR3 comprising the amino acid sequence of SEQ ID NO: 182, and the beta chain comprises a CDR1 comprising the amino acid sequence of SEQ ID NO: 416, a CDR2 comprising the amino acid sequence of SEQ ID NO: 418, and a CDR3 comprising the amino acid sequence of SEQ ID NO: 420;
(aa) the alpha chain comprises a CDR1 comprising the amino acid sequence of SEQ ID NO: 185, a CDR2 comprising the amino acid sequence of SEQ ID NO: 187, and a CDR3 comprising the amino acid sequence of SEQ ID NO: 189, and the beta chain comprises a CDR1 comprising the amino acid sequence of SEQ ID NO: 423, a CDR2 comprising the amino acid sequence of SEQ ID NO: 425, and a CDR3 comprising the amino acid sequence of SEQ ID NO: 427;
(bb) the alpha chain comprises a CDR1 comprising the amino acid sequence of SEQ ID NO: 192, a CDR2 comprising the amino acid sequence of SEQ ID NO: 194, and a CDR3 comprising the amino acid sequence of SEQ ID NO: 196, and the beta chain comprises a CDR1 comprising the amino acid sequence of SEQ ID NO: 430, a CDR2 comprising the amino acid sequence of SEQ ID NO: 432, and a CDR3 comprising the amino acid sequence of SEQ ID NO: 434;
(cc) the alpha chain comprises a CDR1 comprising the amino acid sequence of SEQ ID NO: 199, a CDR2 comprising the amino acid sequence of SEQ ID NO: 201, and a CDR3 comprising the amino acid sequence of SEQ ID NO: 203, and the beta chain comprises a CDR1 comprising the amino acid sequence of SEQ ID NO: 437, a CDR2 comprising the amino acid sequence of SEQ ID NO: 439, and a CDR3 comprising the amino acid sequence of SEQ ID NO: 441;
(dd) the alpha chain comprises a CDR1 comprising the amino acid sequence of SEQ ID NO: 206, a CDR2 comprising the amino acid sequence of SEQ ID NO: 208, and a CDR3 comprising the amino acid sequence of SEQ ID NO: 210, and the beta chain comprises a CDR1 comprising the amino acid sequence of SEQ ID NO: 444, a CDR2 comprising the amino acid sequence of SEQ ID NO: 446, and a CDR3 comprising the amino acid sequence of SEQ ID NO: 448;
(ee) the alpha chain comprises a CDR1 comprising the amino acid sequence of SEQ ID NO: 213, a CDR2 comprising the amino acid sequence of SEQ ID NO: 215, and a CDR3 comprising the amino acid sequence of SEQ ID NO: 217, and the beta chain comprises a CDR1 comprising the amino acid sequence of SEQ ID NO: 451, a CDR2 comprising the amino acid sequence of SEQ ID NO: 453, and a CDR3 comprising the amino acid sequence of SEQ ID NO: 455;
(ff) the alpha chain comprises a CDR1 comprising the amino acid sequence of SEQ ID NO: 220, a CDR2 comprising the amino acid sequence of SEQ ID NO: 222, and a CDR3 comprising the amino acid sequence of SEQ ID NO: 224, and the beta chain comprises a CDR1 comprising the amino acid sequence of SEQ ID NO: 458, a CDR2 comprising the amino acid sequence of SEQ ID NO: 460, and a CDR3 comprising the amino acid sequence of SEQ ID NO: 462;
(gg) the alpha chain comprises a CDR1 comprising the amino acid sequence of SEQ ID NO: 227, a CDR2 comprising the amino acid sequence of SEQ ID NO: 229, and a CDR3 comprising the amino acid sequence of SEQ ID NO: 231, and the beta chain comprises a CDR1 comprising the amino acid sequence of SEQ ID NO: 465, a CDR2 comprising the amino acid sequence of SEQ ID NO: 467, and a CDR3 comprising the amino acid sequence of SEQ ID NO: 469; or
(hh) the alpha chain comprises a CDR1 comprising the amino acid sequence of SEQ ID NO: 234, a CDR2 comprising the amino acid sequence of SEQ ID NO: 236, and a CDR3 comprising the amino acid sequence of SEQ ID NO: 238, and the beta chain comprises a CDR1 comprising the amino acid sequence of SEQ ID NO: 472, a CDR2 comprising the amino acid sequence of SEQ ID NO: 474, and a CDR3 comprising the amino acid sequence of SEQ ID NO: 476.

Embodiment 3. The TCR of embodiment 1 or 2, wherein:
(a) the alpha chain of embodiment 2(a) comprises a variable and joining (VJ) region comprising an amino acid sequence that is at least 90%, 95%, 96%, 97%, 98%, 99% or 100% identical to the amino acid sequence of SEQ ID NO: 479, and the beta chain of embodiment 2(a) comprises a variable, diversity and joining (VDJ) region comprising an amino acid sequence that is at least 90%, 95%, 96%, 97%, 98%, 99% or 100% identical to amino acid sequence of SEQ ID NO: 581;
(b) the alpha chain of embodiment 2(b) comprises a VJ region comprising an amino acid sequence that is at least 90%, 95%, 96%, 97%, 98%, 99% or 100% identical to amino acid sequence of SEQ ID NO: 482, and the beta chain of embodiment 2(b) comprises a VJD region comprising an amino acid sequence that is at least 90%, 95%, 96%, 97%, 98%, 99% or 100% identical to amino acid sequence of SEQ ID NO: 584;
(c) the alpha chain of embodiment 2(c) comprises a VJ region comprising an amino acid sequence that is at least 90%, 95%, 96%, 97%, 98%, 99% or 100% identical to amino acid sequence of SEQ ID NO: 485, and the beta chain of embodiment 2(c) comprises a VJD region comprising an amino acid sequence that is at least 90%, 95%, 96%, 97%, 98%, 99% or 100% identical to amino acid sequence of SEQ ID NO: 587;
(d) the alpha chain of embodiment 2(d) comprises a VJ region comprising an amino acid sequence that is at least 90%, 95%, 96%, 97%, 98%, 99% or 100% identical to amino acid sequence of SEQ ID NO: 488, and the beta chain of embodiment 2(d) comprises a VJD region comprising an amino acid sequence that is at least 90%, 95%, 96%, 97%, 98%, 99% or 100% identical to amino acid sequence of SEQ ID NO: 590;
(e) the alpha chain of embodiment 2(e) comprises a VJ region comprising an amino acid sequence that is at least 90%, 95%, 96%, 97%, 98%, 99% or 100% identical to amino acid sequence of SEQ ID NO: 491, and the beta chain of embodiment 2(e) comprises a VJD region comprising an amino acid sequence that is at least 90%, 95%, 96%, 97%, 98%, 99% or 100% identical to amino acid sequence of SEQ ID NO: 593;
(f) the alpha chain of embodiment 2(f) comprises a VJ region comprising an amino acid sequence that is at least 90%, 95%, 96%, 97%, 98%, 99% or 100% identical to amino acid sequence of SEQ ID NO: 494, and the beta chain of embodiment 2(f) comprises a VJD region comprising an amino acid sequence that is at least 90%, 95%, 96%, 97%, 98%, 99% or 100% identical to amino acid sequence of SEQ ID NO: 596;
(g) the alpha chain of embodiment 2(g) comprises a VJ region comprising an amino acid sequence that is at least 90%, 95%, 96%, 97%, 98%, 99% or 100% identical to amino acid sequence of SEQ ID NO: 497, and the beta chain of embodiment 2(g) comprises a VJD region comprising an amino acid sequence that is at least 90%, 95%, 96%, 97%, 98%, 99% or 100% identical to amino acid sequence of SEQ ID NO: 599;
(h) the alpha chain of embodiment 2(h) comprises a VJ region comprising an amino acid sequence that is at least 90%, 95%, 96%, 97%, 98%, 99% or 100% identical to amino acid sequence of SEQ ID NO: 500, and the beta chain of embodiment 2(h) comprises a VJD region comprising an amino acid sequence that is at least 90%, 95%, 96%, 97%, 98%, 99% or 100% identical to amino acid sequence of SEQ ID NO: 602;
(i) the alpha chain of embodiment 2(i) comprises a VJ region comprising an amino acid sequence that is at least 90%, 95%, 96%, 97%, 98%, 99% or 100% identical to amino acid sequence of SEQ ID NO: 503, and the beta chain of embodiment 2(i) comprises a VJD region comprising an amino acid sequence that is at least 90%, 95%, 96%, 97%, 98%, 99% or 100% identical to amino acid sequence of SEQ ID NO: 605;
(j) the alpha chain of embodiment 2(j) comprises a VJ region comprising an amino acid sequence that is at least 90%, 95%, 96%, 97%, 98%, 99% or 100% identical to amino acid sequence of SEQ ID NO: 506, and the beta chain of embodiment 2(j) comprises a VJD region comprising an amino acid sequence that is at least 90%, 95%, 96%, 97%, 98%, 99% or 100% identical to amino acid sequence of SEQ ID NO: 608;
(k) the alpha chain of embodiment 2(k) comprises a VJ region comprising an amino acid sequence that is at least 90%, 95%, 96%, 97%, 98%, 99% or 100% identical to amino acid sequence of SEQ ID NO: 509, and the beta chain of embodiment 2(k) comprises a VJD region comprising an amino acid sequence that is at least 90%, 95%, 96%, 97%, 98%, 99% or 100% identical to amino acid sequence of SEQ ID NO: 611;
(l) the alpha chain of embodiment 2(l) comprises a VJ region comprising an amino acid sequence that is at least 90%, 95%, 96%, 97%, 98%, 99% or 100% identical to amino acid sequence of SEQ ID NO: 512, and the beta chain of embodiment 2(l) comprises a VJD region comprising an amino acid sequence that is at least 90%, 95%, 96%, 97%, 98%, 99% or 100% identical to amino acid sequence of SEQ ID NO: 614;
(m) the alpha chain of embodiment 2(m) comprises a VJ region comprising an amino acid sequence that is at least 90%, 95%, 96%, 97%, 98%, 99% or 100% identical to amino acid sequence of SEQ ID NO: 515, and the beta chain of embodiment 2(m) comprises a VJD region comprising an amino acid sequence that is at least 90%, 95%, 96%, 97%, 98%, 99% or 100% identical to amino acid sequence of SEQ ID NO: 617;
(n) the alpha chain of embodiment 2(n) comprises a VJ region comprising an amino acid sequence that is at least 90%, 95%, 96%, 97%, 98%, 99% or 100% identical to amino acid sequence of SEQ ID NO: 518, and the beta chain of embodiment 2(n) comprises a VJD region comprising an amino acid sequence that is at least 90%, 95%, 96%, 97%, 98%, 99% or 100% identical to amino acid sequence of SEQ ID NO: 620;
(o) the alpha chain of embodiment 2(o) comprises a VJ region comprising an amino acid sequence that is at least 90%, 95%, 96%, 97%, 98%, 99% or 100% identical to amino acid sequence of SEQ ID NO: 521, and the beta chain of embodiment 2(o) comprises a VJD region comprising an amino acid sequence that is at least 90%, 95%, 96%, 97%, 98%, 99% or 100% identical to amino acid sequence of SEQ ID NO: 623;
(p) the alpha chain of embodiment 2(p) comprises a VJ region comprising an amino acid sequence that is at least 90%, 95%, 96%, 97%, 98%, 99% or 100% identical to amino acid sequence of SEQ ID NO: 524, and the beta chain of embodiment 2(p) comprises a VJD region comprising an amino acid sequence that is at least 90%, 95%, 96%, 97%, 98%, 99% or 100% identical to amino acid sequence of SEQ ID NO: 626;
(q) the alpha chain of embodiment 2(q) comprises a VJ region comprising an amino acid sequence that is at least 90%, 95%, 96%, 97%, 98%, 99% or 100% identical to amino acid sequence of SEQ ID NO: 527, and the beta chain of embodiment 2(q) comprises a VJD region comprising an amino acid sequence that is at least 90%, 95%, 96%, 97%, 98%, 99% or 100% identical to amino acid sequence of SEQ ID NO: 629;
(r) the alpha chain of embodiment 2(r) comprises a VJ region comprising an amino acid sequence that is at least 90%, 95%, 96%, 97%, 98%, 99% or 100% identical to amino acid sequence of SEQ ID NO: 530, and the beta chain of embodiment 2(r) comprises a VJD region comprising an amino acid sequence that is at least 90%, 95%, 96%, 97%, 98%, 99% or 100% identical to amino acid sequence of SEQ ID NO: 632;
(s) the alpha chain of embodiment 2(s) comprises a VJ region comprising an amino acid sequence that is at least 90%, 95%, 96%, 97%, 98%, 99% or 100% identical to amino acid sequence of SEQ ID NO: 533, and the beta chain of embodiment 2(s) comprises a VJD region comprising an amino acid sequence that is at least 90%, 95%, 96%, 97%, 98%, 99% or 100% identical to amino acid sequence of SEQ ID NO: 635;
(t) the alpha chain of embodiment 2(t) comprises a VJ region comprising an amino acid sequence that is at least 90%, 95%, 96%, 97%, 98%, 99% or 100% identical to amino acid sequence of SEQ ID NO: 536, and the beta chain of embodiment 2(t) comprises a VJD region comprising an amino acid sequence that is at least 90%, 95%, 96%, 97%, 98%, 99% or 100% identical to amino acid sequence of SEQ ID NO: 638;
(u) the alpha chain of embodiment 2(u) comprises a VJ region comprising an amino acid sequence that is at least 90%, 95%, 96%, 97%, 98%, 99% or 100% identical to amino acid sequence of SEQ ID NO: 539, and the beta chain of embodiment 2(u) comprises a VJD region comprising an amino acid sequence that is at least 90%, 95%, 96%, 97%, 98%, 99% or 100% identical to amino acid sequence of SEQ ID NO: 641;
(v) the alpha chain of embodiment 2(v) comprises a VJ region comprising an amino acid sequence that is at least 90%, 95%, 96%, 97%, 98%, 99% or 100% identical to amino acid sequence of SEQ ID NO: 542, and the beta chain of embodiment 2(v) comprises a VJD region comprising an amino acid sequence that is at least 90%, 95%, 96%, 97%, 98%, 99% or 100% identical to amino acid sequence of SEQ ID NO: 644;
(w) the alpha chain of embodiment 2(w) comprises a VJ region comprising an amino acid sequence that is at least 90%, 95%, 96%, 97%, 98%, 99% or 100% identical to amino acid sequence of SEQ ID NO: 545, and the beta chain of embodiment 2(w) comprises a VJD region comprising an amino acid sequence that is at least 90%, 95%, 96%, 97%, 98%, 99% or 100% identical to amino acid sequence of SEQ ID NO: 647;
(x) the alpha chain of embodiment 2(x) comprises a VJ region comprising an amino acid sequence that is at least 90%, 95%, 96%, 97%, 98%, 99% or 100% identical to amino acid sequence of SEQ ID NO: 548, and the beta chain of embodiment 2(x) comprises a VJD region comprising an amino acid sequence that is at least 90%, 95%, 96%, 97%, 98%, 99% or 100% identical to amino acid sequence of SEQ ID NO: 650;
(y) the alpha chain of embodiment 2(y) comprises a VJ region comprising an amino acid sequence that is at least 90%, 95%, 96%, 97%, 98%, 99% or 100% identical to amino acid sequence of SEQ ID NO: 551, and the beta chain of embodiment 2(y) comprises a VJD region comprising an amino acid sequence that is at least 90%, 95%, 96%, 97%, 98%, 99% or 100% identical to amino acid sequence of SEQ ID NO: 653;
(z) the alpha chain of embodiment 2(z) comprises a VJ region comprising an amino acid sequence that is at least 90%, 95%, 96%, 97%, 98%, 99% or 100% identical to amino acid sequence of SEQ ID NO: 554, and the beta chain of embodiment 2(z) comprises a VJD region comprising an amino acid sequence that is at least 90%, 95%, 96%, 97%, 98%, 99% or 100% identical to amino acid sequence of SEQ ID NO: 656;
(aa) the alpha chain of embodiment 2(aa) comprises a VJ region comprising an amino acid sequence that is at least 90%, 95%, 96%, 97%, 98%, 99% or 100% identical to amino acid sequence of SEQ ID NO: 557, and the beta chain of embodiment 2(aa) comprises a VJD region comprising an amino acid sequence that is at least 90%, 95%, 96%, 97%, 98%, 99% or 100% identical to amino acid sequence of SEQ ID NO: 659;
(bb) the alpha chain of embodiment 2(bb) comprises a VJ region comprising an amino acid sequence that is at least 90%, 95%, 96%, 97%, 98%, 99% or 100% identical to amino acid sequence of SEQ ID NO: 560, and the beta chain of embodiment 2(bb) comprises a VJD region comprising an amino acid sequence that is at least 90%, 95%, 96%, 97%, 98%, 99% or 100% identical to amino acid sequence of SEQ ID NO: 662;
(cc) the alpha chain of embodiment 2(cc) comprises a VJ region comprising an amino acid sequence that is at least 90%, 95%, 96%, 97%, 98%, 99% or 100% identical to amino acid sequence of SEQ ID NO: 563, and the beta chain of embodiment 2(cc) comprises a VJD region comprising an amino acid sequence that is at least 90%, 95%, 96%, 97%, 98%, 99% or 100% identical to amino acid sequence of SEQ ID NO: 665;
(dd) the alpha chain of embodiment 2(dd) comprises a VJ region comprising an amino acid sequence that is at least 90%, 95%, 96%, 97%, 98%, 99% or 100% identical to amino acid sequence of SEQ ID NO: 566, and the beta chain of embodiment 2(dd) comprises a VJD region comprising an amino acid sequence that is at least 90%, 95%, 96%, 97%, 98%, 99% or 100% identical to amino acid sequence of SEQ ID NO: 668;
(ee) the alpha chain of embodiment 2(ee) comprises a VJ region comprising an amino acid sequence that is at least 90%, 95%, 96%, 97%, 98%, 99% or 100% identical to amino acid sequence of SEQ ID NO: 569, and the beta chain of embodiment 2(ee) comprises a VJD region comprising an amino acid sequence that is at least 90%, 95%, 96%, 97%, 98%, 99% or 100% identical to amino acid sequence of SEQ ID NO: 671;
(ff) the alpha chain of embodiment 2(ff) comprises a VJ region comprising an amino acid sequence that is at least 90%, 95%, 96%, 97%, 98%, 99% or 100% identical to amino acid sequence of SEQ ID NO: 572, and the beta chain of embodiment 2(ff) comprises a VJD region comprising an amino acid sequence that is at least 90%, 95%, 96%, 97%, 98%, 99% or 100% identical to amino acid sequence of SEQ ID NO: 674;
(gg) the alpha chain of embodiment 2(gg) comprises a VJ region comprising an amino acid sequence that is at least 90%, 95%, 96%, 97%, 98%, 99% or 100% identical to amino acid sequence of SEQ ID NO: 575, and the beta chain of embodiment 2(gg) comprises a VJD region comprising an amino acid sequence that is at least 90%, 95%, 96%, 97%, 98%, 99% or 100% identical to amino acid sequence of SEQ ID NO: 677; or
(hh) the alpha chain of embodiment 2(hh) comprises a VJ region comprising an amino acid sequence that is at least 90%, 95%, 96%, 97%, 98%, 99% or 100% identical to amino acid sequence of SEQ ID NO: 578, and the beta chain of embodiment 2(hh) comprises a VJD region comprising an amino acid sequence that is at least 90%, 95%, 96%, 97%, 98%, 99% or 100% identical to amino acid sequence of SEQ ID NO: 680.

Embodiment 4. The TCR of embodiment 1 or 2, wherein:
(a) the alpha chain of embodiment 2(a) comprises an amino acid sequence that is at least 90%, 95%, 96%, 97%, 98%, 99% or 100% identical to the amino acid sequence of SEQ ID NO: 477, and the beta chain of embodiment 2(a) comprises an amino acid sequence that is at least 90%, 95%, 96%, 97%, 98%, 99% or 100% identical to amino acid sequence of SEQ ID NO: 579;
(b) the alpha chain of embodiment 2(b) comprises an amino acid sequence that is at least 90%, 95%, 96%, 97%, 98%, 99% or 100% identical to amino acid sequence of SEQ ID NO: 480, and the beta chain of embodiment 2(b) comprises an amino acid sequence that is at least 90%, 95%, 96%, 97%, 98%, 99% or 100% identical to amino acid sequence of SEQ ID NO: 582;
(c) the alpha chain of embodiment 2(c) comprises an amino acid sequence that is at least 90%, 95%, 96%, 97%, 98%, 99% or 100% identical to amino acid sequence of SEQ ID NO: 483, and the beta chain of embodiment 2(c) comprises an amino acid sequence that is at least 90%, 95%, 96%, 97%, 98%, 99% or 100% identical to amino acid sequence of SEQ ID NO: 585;
(d) the alpha chain of embodiment 2(d) comprises an amino acid sequence that is at least 90%, 95%, 96%, 97%, 98%, 99% or 100% identical to amino acid sequence of SEQ ID NO: 486, and the beta chain of embodiment 2(d) comprises an amino acid sequence that is at least 90%, 95%, 96%, 97%, 98%, 99% or 100% identical to amino acid sequence of SEQ ID NO: 588;
(e) the alpha chain of embodiment 2(e) comprises an amino acid sequence that is at least 90%, 95%, 96%, 97%, 98%, 99% or 100% identical to amino acid sequence of SEQ ID NO: 489, and the beta chain of embodiment 2(e) comprises an amino acid sequence that is at least 90%, 95%, 96%, 97%, 98%, 99% or 100% identical to amino acid sequence of SEQ ID NO: 591;
(f) the alpha chain of embodiment 2(f) comprises an amino acid sequence that is at least 90%, 95%, 96%, 97%, 98%, 99% or 100% identical to amino acid sequence of SEQ ID NO: 492, and the beta chain of embodiment 2(f) comprises an amino acid sequence that is at least 90%, 95%, 96%, 97%, 98%, 99% or 100% identical to amino acid sequence of SEQ ID NO: 594;
(g) the alpha chain of embodiment 2(g) comprises an amino acid sequence that is at least 90%, 95%, 96%, 97%, 98%, 99% or 100% identical to amino acid sequence of SEQ ID NO: 495, and the beta chain of embodiment 2(g) comprises an amino acid sequence that is at least 90%, 95%, 96%, 97%, 98%, 99% or 100% identical to amino acid sequence of SEQ ID NO: 597;
(h) the alpha chain of embodiment 2(h) comprises an amino acid sequence that is at least 90%, 95%, 96%, 97%, 98%, 99% or 100% identical to amino acid sequence of SEQ ID NO: 498, and the beta chain of embodiment 2(h) comprises an amino acid sequence that is at least 90%, 95%, 96%, 97%, 98%, 99% or 100% identical to amino acid sequence of SEQ ID NO: 600;
(i) the alpha chain of embodiment 2(i) comprises an amino acid sequence that is at least 90%, 95%, 96%, 97%, 98%, 99% or 100% identical to amino acid sequence of SEQ ID NO: 501, and the beta chain of embodiment 2(i) comprises an amino acid sequence that is at least 90%, 95%, 96%, 97%, 98%, 99% or 100% identical to amino acid sequence of SEQ ID NO: 603;
(j) the alpha chain of embodiment 2(j) comprises an amino acid sequence that is at least 90%, 95%, 96%, 97%, 98%, 99% or 100% identical to amino acid sequence of SEQ ID NO: 504, and the beta chain of embodiment 2(j) comprises an amino acid sequence that is at least 90%, 95%, 96%, 97%, 98%, 99% or 100% identical to amino acid sequence of SEQ ID NO: 606;
(k) the alpha chain of embodiment 2(k) comprises an amino acid sequence that is at least 90%, 95%, 96%, 97%, 98%, 99% or 100% identical to amino acid sequence of SEQ ID NO: 507, and the beta chain of embodiment 2(k) comprises an amino acid sequence that is at least 90%, 95%, 96%, 97%, 98%, 99% or 100% identical to amino acid sequence of SEQ ID NO: 609;
(l) the alpha chain of embodiment 2(l) comprises an amino acid sequence that is at least 90%, 95%, 96%, 97%, 98%, 99% or 100% identical to amino acid sequence of SEQ ID NO: 510, and the beta chain of embodiment 2(l) comprises an amino acid sequence that is at least 90%, 95%, 96%, 97%, 98%, 99% or 100% identical to amino acid sequence of SEQ ID NO: 612;
(m) the alpha chain of embodiment 2(m) comprises an amino acid sequence that is at least 90%, 95%, 96%, 97%, 98%, 99% or 100% identical to amino acid sequence of SEQ ID NO: 513, and the beta chain of embodiment 2(m) comprises an amino acid sequence that is at least 90%, 95%, 96%, 97%, 98%, 99% or 100% identical to amino acid sequence of SEQ ID NO: 615;
(n) the alpha chain of embodiment 2(n) comprises an amino acid sequence that is at least 90%, 95%, 96%, 97%, 98%, 99% or 100% identical to amino acid sequence of SEQ ID NO: 516, and the beta chain of embodiment 2(n) comprises an amino acid sequence that is at least 90%, 95%, 96%, 97%, 98%, 99% or 100% identical to amino acid sequence of SEQ ID NO: 618;
(o) the alpha chain of embodiment 2(o) comprises an amino acid sequence that is at least 90%, 95%, 96%, 97%, 98%, 99% or 100% identical to amino acid sequence of SEQ ID NO: 519, and the beta chain of embodiment 2(o) comprises an amino acid sequence that is at least 90%, 95%, 96%, 97%, 98%, 99% or 100% identical to amino acid sequence of SEQ ID NO: 621;
(p) the alpha chain of embodiment 2(p) comprises an amino acid sequence that is at least 90%, 95%, 96%, 97%, 98%, 99% or 100% identical to amino acid sequence of SEQ ID NO: 522, and the beta chain of embodiment 2(p) comprises an amino acid sequence that is at least 90%, 95%, 96%, 97%, 98%, 99% or 100% identical to amino acid sequence of SEQ ID NO: 624;
(q) the alpha chain of embodiment 2(q) comprises an amino acid sequence that is at least 90%, 95%, 96%, 97%, 98%, 99% or 100% identical to amino acid sequence of SEQ ID NO: 525, and the beta chain of embodiment 2(q) comprises an amino acid sequence that is at least 90%, 95%, 96%, 97%, 98%, 99% or 100% identical to amino acid sequence of SEQ ID NO: 627;
(r) the alpha chain of embodiment 2(r) comprises an amino acid sequence that is at least 90%, 95%, 96%, 97%, 98%, 99% or 100% identical to amino acid sequence of SEQ ID NO: 528, and the beta chain of embodiment 2(r) comprises an amino acid sequence that is at least 90%, 95%, 96%, 97%, 98%, 99% or 100% identical to amino acid sequence of SEQ ID NO: 630;
(s) the alpha chain of embodiment 2(s) comprises an amino acid sequence that is at least 90%, 95%, 96%, 97%, 98%, 99% or 100% identical to amino acid sequence of SEQ ID NO: 531, and the beta chain of embodiment 2(s) comprises an amino acid sequence that is at least 90%, 95%, 96%, 97%, 98%, 99% or 100% identical to amino acid sequence of SEQ ID NO: 633;
(t) the alpha chain of embodiment 2(t) comprises an amino acid sequence that is at least 90%, 95%, 96%, 97%, 98%, 99% or 100% identical to amino acid sequence of SEQ ID NO: 534, and the beta chain of embodiment 2(t) comprises an amino acid sequence that is at least 90%, 95%, 96%, 97%, 98%, 99% or 100% identical to amino acid sequence of SEQ ID NO: 636;
(u) the alpha chain of embodiment 2(u) comprises an amino acid sequence that is at least 90%, 95%, 96%, 97%, 98%, 99% or 100% identical to amino acid sequence of SEQ ID NO: 537, and the beta chain of embodiment 2(u) comprises an amino acid sequence that is at least 90%, 95%, 96%, 97%, 98%, 99% or 100% identical to amino acid sequence of SEQ ID NO: 639;
(v) the alpha chain of embodiment 2(v) comprises an amino acid sequence that is at least 90%, 95%, 96%, 97%, 98%, 99% or 100% identical to amino acid sequence of SEQ ID NO: 540, and the beta chain of embodiment 2(v) comprises an amino acid sequence that is at least 90%, 95%, 96%, 97%, 98%, 99% or 100% identical to amino acid sequence of SEQ ID NO: 642;
(w) the alpha chain of embodiment 2(w) comprises an amino acid sequence that is at least 90%, 95%, 96%, 97%, 98%, 99% or 100% identical to amino acid sequence of SEQ ID NO: 543, and the beta chain of embodiment 2(w) comprises an amino acid sequence that is at least 90%, 95%, 96%, 97%, 98%, 99% or 100% identical to amino acid sequence of SEQ ID NO: 645;
(x) the alpha chain of embodiment 2(x) comprises an amino acid sequence that is at least 90%, 95%, 96%, 97%, 98%, 99% or 100% identical to amino acid sequence of SEQ ID NO: 546, and the beta chain of embodiment 2(x) comprises an amino acid sequence that is at least 90%, 95%, 96%, 97%, 98%, 99% or 100% identical to amino acid sequence of SEQ ID NO: 648;
(y) the alpha chain of embodiment 2(y) comprises an amino acid sequence that is at least 90%, 95%, 96%, 97%, 98%, 99% or 100% identical to amino acid sequence of SEQ ID NO: 549, and the beta chain of embodiment 2(y) comprises an amino acid sequence that is at least 90%, 95%, 96%, 97%, 98%, 99% or 100% identical to amino acid sequence of SEQ ID NO: 651;
(z) the alpha chain of embodiment 2(z) comprises an amino acid sequence that is at least 90%, 95%, 96%, 97%, 98%, 99% or 100% identical to amino acid sequence of SEQ ID NO: 552, and the beta chain of embodiment 2(z) comprises an amino acid sequence that is at least 90%, 95%, 96%, 97%, 98%, 99% or 100% identical to amino acid sequence of SEQ ID NO: 654;
(aa) the alpha chain of embodiment 2(aa) comprises an amino acid sequence that is at least 90%, 95%, 96%, 97%, 98%, 99% or 100% identical to amino acid sequence of SEQ ID NO: 555, and the beta chain of embodiment 2(aa) comprises an amino acid sequence that is at least 90%, 95%, 96%, 97%, 98%, 99% or 100% identical to amino acid sequence of SEQ ID NO: 657;
(bb) the alpha chain of embodiment 2(bb) comprises an amino acid sequence that is at least 90%, 95%, 96%, 97%, 98%, 99% or 100% identical to amino acid sequence of SEQ ID NO: 558, and the beta chain of embodiment 2(bb) comprises an amino acid sequence that is at least 90%, 95%, 96%, 97%, 98%, 99% or 100% identical to amino acid sequence of SEQ ID NO: 660;
(cc) the alpha chain of embodiment 2(cc) comprises an amino acid sequence that is at least 90%, 95%, 96%, 97%, 98%, 99% or 100% identical to amino acid sequence of SEQ ID NO: 561, and the beta chain of embodiment 2(cc) comprises an amino acid sequence that is at least 90%, 95%, 96%, 97%, 98%, 99% or 100% identical to amino acid sequence of SEQ ID NO: 663;
(dd) the alpha chain of embodiment 2(dd) comprises an amino acid sequence that is at least 90%, 95%, 96%, 97%, 98%, 99% or 100% identical to amino acid sequence of SEQ ID NO: 564, and the beta chain of embodiment 2(dd) comprises an amino acid sequence that is at least 90%, 95%, 96%, 97%, 98%, 99% or 100% identical to amino acid sequence of SEQ ID NO: 666;
(ee) the alpha chain of embodiment 2(ee) comprises an amino acid sequence that is at least 90%, 95%, 96%, 97%, 98%, 99% or 100% identical to amino acid sequence of SEQ ID NO: 567, and the beta chain of embodiment 2(ee) comprises an amino acid sequence that is at least 90%, 95%, 96%, 97%, 98%, 99% or 100% identical to amino acid sequence of SEQ ID NO: 669;
(ff) the alpha chain of embodiment 2(ff) comprises an amino acid sequence that is at least 90%, 95%, 96%, 97%, 98%, 99% or 100% identical to amino acid sequence of SEQ ID NO: 570, and the beta chain of embodiment 2(ff) comprises an amino acid sequence that is at least 90%, 95%, 96%, 97%, 98%, 99% or 100% identical to amino acid sequence of SEQ ID NO: 672;
(gg) the alpha chain of embodiment 2(gg) comprises an amino acid sequence that is at least 90%, 95%, 96%, 97%, 98%, 99% or 100% identical to amino acid sequence of SEQ ID NO: 573, and the beta chain of embodiment 2(gg) comprises an amino acid sequence that is at least 90%, 95%, 96%, 97%, 98%, 99% or 100% identical to amino acid sequence of SEQ ID NO: 675; or
(hh) the alpha chain of embodiment 2(hh) comprises an amino acid sequence that is at least 90%, 95%, 96%, 97%, 98%, 99% or 100% identical to amino acid sequence of SEQ ID NO: 576, and the beta chain of embodiment 2(hh) comprises an amino acid sequence that is at least 90%, 95%, 96%, 97%, 98%, 99% or 100% identical to amino acid sequence of SEQ ID NO: 678.

Embodiment 5. A nucleic acid molecule encoding the TCR of any one of embodiments 1-4.

Embodiment 6. A vector comprising the nucleic acid molecule of embodiment 5.

Embodiment 7. A cell transformed to express the nucleic acid molecule of embodiment 5.

Embodiment 8. A cell comprising the vector of embodiment 6.

Embodiment 9. The cell of embodiment 7 or 8, wherein the cell is a CD8+ T cell.

Embodiment 10. A pharmaceutical composition comprising the TCR of any one of embodiment 1-4, the nucleic acid molecule of embodiment 5, the vector of embodiment 6, or the cell of any one of embodiments 7-9.

What is claimed:

1. A T-cell receptor (TCR) comprising an alpha chain and a beta chain, wherein:
(a) the alpha chain comprises a complementarity determining region 1 (CDR1) comprising the amino acid sequence of SEQ ID NO: 3, a complementarity determining region 2 (CDR2) comprising the amino acid sequence of SEQ ID NO: 5, and a complementarity determining region 3 (CDR3) comprising the amino acid sequence of SEQ ID NO: 7, and the beta chain comprises a CDR1 comprising the amino acid sequence of SEQ ID NO: 241, a CDR2 comprising the amino acid sequence of SEQ ID NO: 243, and a CDR3 comprising the amino acid sequence of SEQ ID NO: 245;
(b) the alpha chain comprises a CDR1 comprising the amino acid sequence of SEQ ID NO: 10, a CDR2 comprising the amino acid sequence of SEQ ID NO: 12, and a CDR3 comprising the amino acid sequence of SEQ ID NO: 14, and the beta chain comprises a CDR1 comprising the amino acid sequence of SEQ ID NO: 248, a CDR2 comprising the amino acid sequence of SEQ ID NO: 250, and a CDR3 comprising the amino acid sequence of SEQ ID NO: 252;
(c) the alpha chain comprises a CDR1 comprising the amino acid sequence of SEQ ID NO: 17, a CDR2 comprising the amino acid sequence of SEQ ID NO: 19, and a CDR3 comprising the amino acid sequence of SEQ ID NO: 21, and the beta chain comprises a CDR1 comprising the amino acid sequence of SEQ ID NO: 255, a CDR2 comprising the amino acid sequence of SEQ ID NO: 257, and a CDR3 comprising the amino acid sequence of SEQ ID NO: 259;
(d) the alpha chain comprises a CDR1 comprising the amino acid sequence of SEQ ID NO: 52, a CDR2 comprising the amino acid sequence of SEQ ID NO: 54, and a CDR3 comprising the amino acid sequence of SEQ ID NO: 56, and the beta chain comprises a CDR1 comprising the amino acid sequence of SEQ ID NO: 290, a CDR2 comprising the amino acid sequence of SEQ ID NO: 292, and a CDR3 comprising the amino acid sequence of SEQ ID NO: 294;
(e) the alpha chain comprises a CDR1 comprising the amino acid sequence of SEQ ID NO: 87, a CDR2 comprising the amino acid sequence of SEQ ID NO: 89, and a CDR3 comprising the amino acid sequence of SEQ ID NO: 91, and the beta chain comprises a CDR1 comprising the amino acid sequence of SEQ ID NO: 325, a CDR2 comprising the amino acid sequence of SEQ ID NO: 327, and a CDR3 comprising the amino acid sequence of SEQ ID NO: 329;
(f) the alpha chain comprises a CDR1 comprising the amino acid sequence of SEQ ID NO: 94, a CDR2 comprising the amino acid sequence of SEQ ID NO: 96, and a CDR3 comprising the amino acid sequence of SEQ ID NO: 98, and the beta chain comprises a CDR1 comprising the amino acid sequence of SEQ ID NO: 332, a CDR2 comprising the amino acid sequence of SEQ ID NO: 334, and a CDR3 comprising the amino acid sequence of SEQ ID NO: 336;
(g) the alpha chain comprises a CDR1 comprising the amino acid sequence of SEQ ID NO: 150, a CDR2 comprising the amino acid sequence of SEQ ID NO: 152, and a CDR3 comprising the amino acid sequence of SEQ ID NO: 154, and the beta chain comprises a CDR1 comprising the amino acid sequence of SEQ ID NO: 388, a CDR2 comprising the amino acid sequence of SEQ ID NO: 390, and a CDR3 comprising the amino acid sequence of SEQ ID NO: 392;
(h) the alpha chain comprises a CDR1 comprising the amino acid sequence of SEQ ID NO: 171, a CDR2 comprising the amino acid sequence of SEQ ID NO: 173, and a CDR3 comprising the amino acid sequence of SEQ ID NO: 175, and the beta chain comprises a CDR1 comprising the amino acid sequence of SEQ ID NO: 409, a CDR2 comprising the amino acid sequence of SEQ ID NO: 411, and a CDR3 comprising the amino acid sequence of SEQ ID NO: 413;
(i) the alpha chain comprises a CDR1 comprising the amino acid sequence of SEQ ID NO: 185, a CDR2 comprising the amino acid sequence of SEQ ID NO: 187, and a CDR3 comprising the amino acid sequence of SEQ ID NO: 189, and the beta chain comprises a CDR1 comprising the amino acid sequence of SEQ ID NO: 423, a CDR2 comprising the amino acid sequence of SEQ ID NO: 425, and a CDR3 comprising the amino acid sequence of SEQ ID NO: 427;
(j) the alpha chain comprises a CDR1 comprising the amino acid sequence of SEQ ID NO: 192, a CDR2 comprising the amino acid sequence of SEQ ID NO: 194, and a CDR3 comprising the amino acid sequence of SEQ ID NO: 196, and the beta chain comprises a CDR1 comprising the amino acid sequence of SEQ ID NO: 430, a CDR2 comprising the amino acid sequence of SEQ ID NO: 432, and a CDR3 comprising the amino acid sequence of SEQ ID NO: 434;
(k) the alpha chain comprises a CDR1 comprising the amino acid sequence of SEQ ID NO: 206, a CDR2 comprising the amino acid sequence of SEQ ID NO: 208, and a CDR3 comprising the amino acid sequence of SEQ ID NO: 210, and the beta chain comprises a CDR1 comprising the amino acid sequence of SEQ ID NO: 444, a CDR2 comprising the amino acid sequence of SEQ ID NO: 446, and a CDR3 comprising the amino acid sequence of SEQ ID NO: 448;
(l) the alpha chain comprises a CDR1 comprising the amino acid sequence of SEQ ID NO: 213, a CDR2 comprising the amino acid sequence of SEQ ID NO: 215, and a CDR3 comprising the amino acid sequence of SEQ ID NO: 217, and the beta chain comprises a CDR1 comprising the amino acid sequence of SEQ ID NO: 451, a CDR2 comprising the amino acid sequence of SEQ ID NO: 453, and a CDR3 comprising the amino acid sequence of SEQ ID NO: 455.

2. The TCR of claim 1, wherein:
(a) the alpha chain of claim 1(a) comprises a variable and joining (VJ) region comprising an amino acid sequence that is at least 90%, 95%, 96%, 97%, 98%, 99%, or 100% identical to the amino acid sequence of SEQ ID NO: 479, and the beta chain of claim 1(a) comprises a variable, diversity and joining (VDJ) region comprising an amino acid sequence that is at least 90%, 95%, 96%, 97%, 98%, 99%, or 100% identical to the amino acid sequence of SEQ ID NO: 581;
(b) the alpha chain of claim 1(b) comprises a VJ region comprising an amino acid sequence that is at least 90%, 95%, 96%, 97%, 98%, 99%, or 100% identical to the amino acid sequence of SEQ ID NO: 482, and the beta chain of claim 1(b) comprises a VJD region comprising an amino acid sequence that is at least 90%, 95%, 96%, 97%, 98%, 99%, or 100% identical to the amino acid sequence of SEQ ID NO: 584;
(c) the alpha chain of claim 1(c) comprises a VJ region comprising an amino acid sequence that is at least 90%, 95%, 96%, 97%, 98%, 99%, or 100% identical to the amino acid sequence of SEQ ID NO: 485, and the beta chain of claim 1(c) comprises a VJD region comprising an amino acid sequence that is at least 90%, 95%, 96%, 97%, 98%, 99%, or 100% identical to the amino acid sequence of SEQ ID NO: 587;
(d) the alpha chain of claim 1(d) comprises a VJ region comprising an amino acid sequence that is at least 90%, 95%, 96%, 97%, 98%, 99%, or 100% identical to the amino acid sequence of SEQ ID NO: 500, and the beta chain of claim 1(d) comprises a VJD region comprising an amino acid sequence that is at least 90%, 95%, 96%, 97%, 98%, 99%, or 100% identical to the amino acid sequence of SEQ ID NO: 602;
(e) the alpha chain of claim 1(e) comprises a VJ region comprising an amino acid sequence that is at least 90%, 95%, 96%, 97%, 98%, 99%, or 100% identical to the amino acid sequence of SEQ ID NO: 515, and the beta chain of claim 1(e) comprises a VJD region comprising an amino acid sequence that is at least 90%, 95%, 96%, 97%, 98%, 99%, or 100% identical to the amino acid sequence of SEQ ID NO: 617;
(f) the alpha chain of claim 1(f) comprises a VJ region comprising an amino acid sequence that is at least 90%, 95%, 96%, 97%, 98%, 99%, or 100% identical to the amino acid sequence of SEQ ID NO: 518, and the beta chain of claim 1(f) comprises a VJD region comprising an amino acid sequence that is at least 90%, 95%, 96%, 97%, 98%, 99%, or 100% identical to the amino acid sequence of SEQ ID NO: 620;
(g) the alpha chain of claim 1(g) comprises a VJ region comprising an amino acid sequence that is at least 90%, 95%, 96%, 97%, 98%, 99%, or 100% identical to the amino acid sequence of SEQ ID NO: 542, and the beta chain of claim 1(g) comprises a VJD region comprising an amino acid sequence that is at least 90%, 95%, 96%, 97%, 98%, 99%, or 100% identical to the amino acid sequence of SEQ ID NO: 644;
(h) the alpha chain of claim 1(h) comprises a VJ region comprising an amino acid sequence that is at least 90%, 95%, 96%, 97%, 98%, 99%, or 100% identical to the amino acid sequence of SEQ ID NO: 551, and the beta chain of claim 1(h) comprises a VJD region comprising an amino acid sequence that is at least 90%, 95%, 96%, 97%, 98%, 99%, or 100% identical to the amino acid sequence of SEQ ID NO: 653;
(i) the alpha chain of claim 1(i) comprises a VJ region comprising an amino acid sequence that is at least 90%, 95%, 96%, 97%, 98%, 99%, or 100% identical to the amino acid sequence of SEQ ID NO: 557, and the beta chain of claim 1(i) comprises a VJD region comprising an amino acid sequence that is at least 90%, 95%, 96%, 97%, 98%, 99%, or 100% identical to the amino acid sequence of SEQ ID NO: 659;
(j) the alpha chain of claim 1(j) comprises a VJ region comprising an amino acid sequence that is at least 90%, 95%, 96%, 97%, 98%, 99%, or 100% identical to the amino acid sequence of SEQ ID NO: 560, and the beta chain of claim 1(j) comprises a VJD region comprising an amino acid sequence that is at least 90%, 95%, 96%, 97%, 98%, 99%, or 100% identical to the amino acid sequence of SEQ ID NO: 662;
(k) the alpha chain of claim 1(k) comprises a VJ region comprising an amino acid sequence that is at least 90%, 95%, 96%, 97%, 98%, 99%, or 100% identical to the amino acid sequence of SEQ ID NO: 566, and the beta chain of claim 1(k) comprises a VJD region comprising an amino acid sequence that is at least 90%, 95%, 96%, 97%, 98%, 99%, or 100% identical to the amino acid sequence of SEQ ID NO: 668;
(l) the alpha chain of claim 1(l) comprises a VJ region comprising an amino acid sequence that is at least 90%, 95%, 96%, 97%, 98%, 99%, or 100% identical to the amino acid sequence of SEQ ID NO: 569, and the beta chain of claim 1(l) comprises a VJD region comprising an amino acid sequence that is at least 90%, 95%, 96%, 97%, 98%, 99%, or 100% identical to the amino acid sequence of SEQ ID NO: 671.

3. The TCR of claim 1, wherein:
(a) the alpha chain of claim 1(a) comprises an amino acid sequence that is at least 90%, 95%, 96%, 97%, 98%, 99%, or 100% identical to the amino acid sequence of SEQ ID NO: 477, and the beta chain of claim 1(a) comprises an amino acid sequence that is at least 90%, 95%, 96%, 97%, 98%, 99%, or 100% identical to the amino acid sequence of SEQ ID NO: 579;
(b) the alpha chain of claim 1(b) comprises an amino acid sequence that is at least 90%, 95%, 96%, 97%, 98%, 99%, or 100% identical to the amino acid sequence of SEQ ID NO: 480, and the beta chain of claim 1(b) comprises an amino acid sequence that is at least 90%, 95%, 96%, 97%, 98%, 99%, or 100% identical to the amino acid sequence of SEQ ID NO: 582;
(c) the alpha chain of claim 1(c) comprises an amino acid sequence that is at least 90%, 95%, 96%, 97%, 98%, 99%, or 100% identical to the amino acid sequence of SEQ ID NO: 483, and the beta chain of claim 1(c) comprises an amino acid sequence that is at least 90%, 95%, 96%, 97%, 98%, 99%, or 100% identical to the amino acid sequence of SEQ ID NO: 585;
(d) the alpha chain of claim 1(d) comprises an amino acid sequence that is at least 90%, 95%, 96%, 97%, 98%, 99%, or 100% identical to the amino acid sequence of SEQ ID NO: 498, and the beta chain of claim 1(d) comprises an amino acid sequence that is at least 90%, 95%, 96%, 97%, 98%, 99%, or 100% identical to the amino acid sequence of SEQ ID NO: 600;
(e) the alpha chain of claim 1(e) comprises an amino acid sequence that is at least 90%, 95%, 96%, 97%, 98%, 99%, or 100% identical to the amino acid sequence of SEQ ID NO: 513, and the beta chain of claim 1(e) comprises an amino acid sequence that is at least 90%, 95%, 96%, 97%, 98%, 99%, or 100% identical to the amino acid sequence of SEQ ID NO: 615;
(f) the alpha chain of claim 1(f) comprises an amino acid sequence that is at least 90%, 95%, 96%, 97%, 98%, 99%, or 100% identical to the amino acid sequence of SEQ ID NO: 516, and the beta chain of claim 1(f) comprises an amino acid sequence that is at least 90%, 95%, 96%, 97%, 98%, 99%, or 100% identical to the amino acid sequence of SEQ ID NO: 618;
(g) the alpha chain of claim 1(g) comprises an amino acid sequence that is at least 90%, 95%, 96%, 97%, 98%, 99%, or 100% identical to the amino acid sequence of SEQ ID NO: 540, and the beta chain of claim 1(g) comprises an amino acid sequence that is at least 90%, 95%, 96%, 97%, 98%, 99%, or 100% identical to the amino acid sequence of SEQ ID NO: 642;
(h) the alpha chain of claim 1(h) comprises an amino acid sequence that is at least 90%, 95%, 96%, 97%, 98%, 99%, or 100% identical to the amino acid sequence of SEQ ID NO: 549, and the beta chain of claim 1(h) comprises an amino acid sequence that is at least 90%, 95%, 96%, 97%, 98%, 99%, or 100% identical to the amino acid sequence of SEQ ID NO: 651;
(i) the alpha chain of claim 1(i) comprises an amino acid sequence that is at least 90%, 95%, 96%, 97%, 98%, 99%, or 100% identical to the amino acid sequence of SEQ ID NO: 555, and the beta chain of claim 1(i) comprises an amino acid sequence that is at least 90%, 95%, 96%, 97%, 98%, 99%, or 100% identical to the amino acid sequence of SEQ ID NO: 657;
(j) the alpha chain of claim 1(j) comprises an amino acid sequence that is at least 90%, 95%, 96%, 97%, 98%, 99%, or 100% identical to the amino acid sequence of SEQ ID NO: 558, and the beta chain of claim 1(j) comprises an amino acid sequence that is at least 90%, 95%, 96%, 97%, 98%, 99%, or 100% identical to the amino acid sequence of SEQ ID NO: 660;
(k) the alpha chain of claim 1(k) comprises an amino acid sequence that is at least 90%, 95%, 96%, 97%, 98%, 99%, or 100% identical to the amino acid sequence of SEQ ID NO: 564, and the beta chain of claim 1(k) comprises an amino acid sequence that is at least 90%, 95%, 96%, 97%, 98%, 99%, or 100% identical to the amino acid sequence of SEQ ID NO: 666;
(l) the alpha chain of claim 1(l) comprises an amino acid sequence that is at least 90%, 95%, 96%, 97%, 98%, 99%, or 100% identical to the amino acid sequence of SEQ ID NO: 567, and the beta chain of claim 1(l) comprises an amino acid sequence that is at least 90%, 95%, 96%, 97%, 98%, 99%, or 100% identical to the amino acid sequence of SEQ ID NO: 669.

4. A nucleic acid molecule encoding the TCR of claim 1.

5. A nucleic acid molecule encoding the TCR of claim 2.

6. A nucleic acid molecule encoding the TCR of claim 3.

7. A vector comprising the nucleic acid molecule of claim 4.

8. A vector comprising the nucleic acid molecule of claim 5.

9. A vector comprising the nucleic acid molecule of claim 6.

10. A cell transformed to express the nucleic acid molecule of claim 4.

11. A cell transformed to express the nucleic acid molecule of claim 5.

12. A cell transformed to express the nucleic acid molecule of claim 6.

13. A cell comprising the vector of claim 7.

14. A cell comprising the vector of claim 8.

15. A cell comprising the vector of claim 9.

16. The cell of claim 10, wherein the cell is a CD8+ T cell.

17. The cell of claim 11, wherein the cell is a CD8+ T cell.

18. The cell of claim 12, wherein the cell is a CD8+ T cell.

19. A pharmaceutical composition comprising the TCR of claim 1.

20. A pharmaceutical composition comprising the TCR of claim 2.

21. A pharmaceutical composition comprising the TCR of claim 3.


SEQUENCE LISTING (706 sequences)

SEQ ID NO: 1 - DNA, 955 bp, synthetic construct
  misc_feature 1..955: note = Description of Artificial Sequence: Synthetic polynucleotide
  source 1..955: mol_type = other DNA
ggggcaggatgtgattctaattggttggaacatcttttgaaatcgtgtttctgtagagaaagaaaaactaccatatttggatagccctggccaactttcaaggctcctaaatctgagttttcagtgaactggacagaaaaaaaaaatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgcccgatcttataacaccgacaagctcatctttgggactgggaccagattacaagtctttccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 2 - DNA, 15 bp, synthetic construct
  misc_feature 1..15: note = Description of Artificial Sequence: Synthetic oligonucleotide
  source 1..15: mol_type = other DNA
accacttcagacaga

SEQ ID NO: 3 - protein, 5 aa, synthetic construct
  REGION 1..5: note = Description of Artificial Sequence: Synthetic peptide
TTSDR

SEQ ID NO: 4 - DNA, 15 bp, synthetic construct
  misc_feature 1..15: note = Description of Artificial Sequence: Synthetic oligonucleotide
  source 1..15: mol_type = other DNA
accacttcagacaga

SEQ ID NO: 5 - protein, 7 aa, synthetic construct
  REGION 1..7: note = Description of Artificial Sequence: Synthetic peptide
LLSNGAV

SEQ ID NO: 6 - DNA, 33 bp, synthetic construct
  misc_feature 1..33: note = Description of Artificial Sequence: Synthetic oligonucleotide
  source 1..33: mol_type = other DNA
gccgcccgatcttataacaccgacaagctcatc

SEQ ID NO: 7 - protein, 13 aa, synthetic construct
  REGION 1..13: note = Description of Artificial Sequence: Synthetic peptide
CAARSYNTDKLIF

SEQ ID NO: 8 - DNA, 1069 bp, synthetic construct
  misc_feature 1..1069: note = Description of Artificial Sequence: Synthetic polynucleotide
  source 1..1069: mol_type = other DNA
tggggagtcttgctcctcacagagctttgaggagctggatcaaaattgtgctccacagagagaagataccggtgtcgggaagcaccagtgccctgaggaagggccatttccaaaagccctgtgctgacacagggttgctggttcctcttcaagagcccactctctggggtggggccatatctccagcagaggtgggctggaaaggacccccccaatcccgcccgccgtgagcttagctggagccatggcctctgcacccatctcgatgcttgcgatgctcttcacattgagtgggctgagagctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacatggggggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 9 - DNA, 18 bp, synthetic construct
  misc_feature 1..18: note = Description of Artificial Sequence: Synthetic oligonucleotide
  source 1..18: mol_type = other DNA
gtctctggaaacccttat

SEQ ID NO: 10 - protein, 6 aa, synthetic construct
  REGION 1..6: note = Description of Artificial Sequence: Synthetic peptide
VSGNPY

SEQ ID NO: 11 - DNA, 24 bp, synthetic construct
  misc_feature 1..24: note = Description of Artificial Sequence: Synthetic oligonucleotide
  source 1..24: mol_type = other DNA
tacatcacaggggataacctggtt

SEQ ID NO: 12 - protein, 8 aa, synthetic construct
  REGION 1..8: note = Description of Artificial Sequence: Synthetic peptide
YITGDNLV

SEQ ID NO: 13 - DNA, 39 bp, synthetic construct
  misc_feature 1..39: note = Description of Artificial Sequence: Synthetic oligonucleotide
  source 1..39: mol_type = other DNA
gctgtgagagacatggggggttctgcaaggcaactgacc

SEQ ID NO: 14 - protein, 15 aa, synthetic construct
  REGION 1..15: note = Description of Artificial Sequence: Synthetic peptide
CAVRDMGGSARQLTF

SEQ ID NO: 15 - DNA, 920 bp, synthetic construct
  misc_feature 1..920: note = Description of Artificial Sequence: Synthetic polynucleotide
  source 1..920: mol_type = other DNA
gagcctgagtgacagctgctggtgtgggccctggcagttgctgctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtggggacgttttctggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 16 - DNA, 21 bp, synthetic construct
  misc_feature 1..21: note = Description of Artificial Sequence: Synthetic oligonucleotide
  source 1..21: mol_type = other DNA
aacattgctacaaatgattat

SEQ ID NO: 17 - protein, 7 aa, synthetic construct
  REGION 1..7: note = Description of Artificial Sequence: Synthetic peptide
NIATNDY

SEQ ID NO: 18 - DNA, 15 bp, synthetic construct
  misc_feature 1..15: note = Description of Artificial Sequence: Synthetic oligonucleotide
  source 1..15: mol_type = other DNA
ggatacaagacaaaa

SEQ ID NO: 19 - protein, 5 aa, synthetic construct
  REGION 1..5: note = Description of Artificial Sequence: Synthetic peptide
GYKTK

SEQ ID NO: 20 - DNA, 39 bp, synthetic construct
  misc_feature 1..39: note = Description of Artificial Sequence: Synthetic oligonucleotide
  source 1..39: mol_type = other DNA
ctcgtggggacgttttctggttctgcaaggcaactgacc

SEQ ID NO: 21 - protein, 15 aa, synthetic construct
  REGION 1..15: note = Description of Artificial Sequence: Synthetic peptide
CLVGTFSGSARQLTF

SEQ ID NO: 22 - DNA, 1088 bp, synthetic construct
  misc_feature 1..1088: note = Description of Artificial Sequence: Synthetic polynucleotide
  source 1..1088: mol_type = other DNA
ggcataataaatgtctacgcctcatgccactaggtggcaatgtgggtgttatactgaaaagatcacagatggttcactttgcaagtaaaactgtaaatgttcttaagtgtgcatttctgctgcttctgatgggctgaaaatcccctttgatttctaaagtaaatgtagagacgttttaaaaataaaggactcctttgtccaagatatattccgaaatcctccaacagagacctgtgtgagcttctgctgcagtaataatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagcacctctatgtattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 23 - DNA, 15 bp, synthetic construct
  misc_feature 1..15: note = Description of Artificial Sequence: Synthetic oligonucleotide
  source 1..15: mol_type = other DNA
gtaggaataagtgcc

SEQ ID NO: 24 - protein, 5 aa, synthetic construct
  REGION 1..5: note = Description of Artificial Sequence: Synthetic peptide
VGISA

SEQ ID NO: 25 - DNA, 15 bp, synthetic construct
  misc_feature 1..15: note = Description of Artificial Sequence: Synthetic oligonucleotide
  source 1..15: mol_type = other DNA
ctgagctcagggaag

SEQ ID NO: 26 - protein, 5 aa, synthetic construct
  REGION 1..5: note = Description of Artificial Sequence: Synthetic peptide
LSSGK

SEQ ID NO: 27 - DNA, 48 bp, synthetic construct
  misc_feature 1..48: note = Description of Artificial Sequence: Synthetic oligonucleotide
  source 1..48: mol_type = other DNA
gctgtcagcacctctatgtattcaggaggaggtgctgacggactcacc

SEQ ID NO: 28 - protein, 18 aa, synthetic construct
  REGION 1..18: note = Description of Artificial Sequence: Synthetic peptide
CAVSTSMYSGGGADGLTF

SEQ ID NO: 29 - DNA, 971 bp, synthetic construct
  misc_feature 1..971: note = Description of Artificial Sequence: Synthetic polynucleotide
  source 1..971: mol_type = other DNA
ggcttagtggtgaattaggggtgttaaaaagagcatcatttttttgaactggtaaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaagggagggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 30 - DNA, 18 bp, synthetic construct
  misc_feature 1..18: note = Description of Artificial Sequence: Synthetic oligonucleotide
  source 1..18: mol_type = other DNA
gaccgaggttcccagtcc

SEQ ID NO: 31 - protein, 6 aa, synthetic construct
  REGION 1..6: note = Description of Artificial Sequence: Synthetic peptide
DRGSQS

SEQ ID NO: 32 - DNA, 18 bp, synthetic construct
  misc_feature 1..18: note = Description of Artificial Sequence: Synthetic oligonucleotide
  source 1..18: mol_type = other DNA
atatactccaatggtgac

SEQ ID NO: 33 - protein, 6 aa, synthetic construct
  REGION 1..6: note = Description of Artificial Sequence: Synthetic peptide
IYSNGD

SEQ ID NO: 34 - DNA, 27 bp, synthetic construct
  misc_feature 1..27: note = Description of Artificial Sequence: Synthetic oligonucleotide
  source 1..27: mol_type = other DNA
gccgtgaagggagggtatgcactcaac

SEQ ID NO: 35 - protein, 11 aa, synthetic construct
  REGION 1..11: note = Description of Artificial Sequence: Synthetic peptide
CAVKGGYALNF

SEQ ID NO: 36 - DNA, 931 bp, synthetic construct
  misc_feature 1..931: note = Description of Artificial Sequence: Synthetic polynucleotide
  source 1..931: mol_type = other DNA
ggcccatttcctgttctgaagcagctacggcaccagtgcagctgatactcaaggttcagatcagaagaggaggcttctcaccctgcagcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttatagtccccccctccccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 37 - DNA, 21 bp, synthetic construct
  misc_feature 1..21: note = Description of Artificial Sequence: Synthetic oligonucleotide
  source 1..21: mol_type = other DNA
accagtgagagtgattattat

SEQ ID NO: 38 - protein, 7 aa, synthetic construct
  REGION 1..7: note = Description of Artificial Sequence: Synthetic peptide
TSESDYY

SEQ ID NO: 39 - DNA, 24 bp, synthetic construct
  misc_feature 1..24: note = Description of Artificial Sequence: Synthetic oligonucleotide
  source 1..24: mol_type = other DNA
caagaagcttataagcaacagaat

SEQ ID NO: 40 - protein, 8 aa, synthetic construct
  REGION 1..8: note = Description of Artificial Sequence: Synthetic peptide
QEAYKQQN

SEQ ID NO: 41 - DNA, 36 bp, synthetic construct
  misc_feature 1..36: note = Description of Artificial Sequence: Synthetic oligonucleotide
  source 1..36: mol_type = other DNA
gcttatagtccccccctccccggtaaccagttctat

SEQ ID NO: 42 - protein, 14 aa, synthetic construct
  REGION 1..14: note = Description of Artificial Sequence: Synthetic peptide
CAYSPPLPGNQFYF

SEQ ID NO: 43 - DNA, 925 bp, synthetic construct
  misc_feature 1..925: note = Description of Artificial Sequence: Synthetic polynucleotide
  source 1..925: mol_type = other DNA
gcttttttgaaatcgtgtttctgtagagaaagaaaaactaccatatttggatagccctggccaactttcaaggctcctaaatctgagttttcagtgaactggacagaaaaaaaaaatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccggtgaacctgatagcaactatcagttaatctggggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 44 - DNA, 15 bp, synthetic construct
  misc_feature 1..15: note = Description of Artificial Sequence: Synthetic oligonucleotide
  source 1..15: mol_type = other DNA
accacttcagacaga

SEQ ID NO: 45 - protein, 5 aa, synthetic construct
  REGION 1..5: note = Description of Artificial Sequence: Synthetic peptide
TTSDR

SEQ ID NO: 46 - DNA, 21 bp, synthetic construct
  misc_feature 1..21: note = Description of Artificial Sequence: Synthetic oligonucleotide
  source 1..21: mol_type = other DNA
ttgctatcaaatggagcagtg

SEQ ID NO: 47 - protein, 7 aa, synthetic construct
  REGION 1..7: note = Description of Artificial Sequence: Synthetic peptide
LLSNGAV

SEQ ID NO: 48 - DNA, 33 bp, synthetic construct
  misc_feature 1..33: note = Description of Artificial Sequence: Synthetic oligonucleotide
  source 1..33: mol_type = other DNA
gccggtgaacctgatagcaactatcagttaatc

SEQ ID NO: 49 - protein, 13 aa, synthetic construct
  REGION 1..13: note = Description of Artificial Sequence: Synthetic peptide
CAGEPDSNYQLIW

SEQ ID NO: 50 - DNA, 1034 bp, synthetic construct
  misc_feature 1..1034: note = Description of Artificial Sequence: Synthetic polynucleotide
  source 1..1034: mol_type = other DNA
agagagagagagagagagaggaggaggaggaggaggaggaaggatgaggggaggggcttagtggtgaattaggggtgttaaaaagagcatcatttttttgaactggtaaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtggggggggccgggggttaccagaaagttacctttggaactggaacaaagctccaagtcatcccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 51 - DNA, 18 bp, synthetic construct
  misc_feature 1..18: note = Description of Artificial Sequence: Synthetic oligonucleotide
  source 1..18: mol_type = other DNA
gaccgaggttcccagtcc

SEQ ID NO: 52 - protein, 6 aa, synthetic construct
  REGION 1..6: note = Description of Artificial Sequence: Synthetic peptide
DRGSQS

SEQ ID NO: 53 - DNA, 18 bp, synthetic construct
  misc_feature 1..18: note = Description of Artificial Sequence: Synthetic oligonucleotide
  source 1..18: mol_type = other DNA
atatactccaatggtgac

SEQ ID NO: 54 - protein, 6 aa, synthetic construct
  REGION 1..6: note = Description of Artificial Sequence: Synthetic peptide
IYSNGD

SEQ ID NO: 55 - DNA, 36 bp, synthetic construct
  misc_feature 1..36: note = Description of Artificial Sequence: Synthetic oligonucleotide
  source 1..36: mol_type = other DNA
gccgtggggggggccgggggttaccagaaagttacc

SEQ ID NO: 56 - protein, 14 aa, synthetic construct
  REGION 1..14: note = Description of Artificial Sequence: Synthetic peptide
CAVGGAGGYQKVTF

SEQ ID NO: 57 - DNA, 953 bp, synthetic construct
  misc_feature 1..953: note = Description of Artificial Sequence: Synthetic polynucleotide
  source 1..953: mol_type = other DNA
gtcctccctttgtatttctactgggttttgcatccggactgatcttccttccctcacccacatgaagtgtctaccttctgcagactacagtggctcaggaaccggggatgcagtgccaggctcatggtatcctgcagcagatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgagagatcctctttctggtggctacaataagctgatttttggagcagggaccaggctggctgtacacccatatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 58 - DNA, 18 bp, synthetic construct
  misc_feature 1..18: note = Description of Artificial Sequence: Synthetic oligonucleotide
  source 1..18: mol_type = other DNA
acatctgggttttatggg

SEQ ID NO: 59 - protein, 6 aa, synthetic construct
  REGION 1..6: note = Description of Artificial Sequence: Synthetic peptide
TSGFYG

SEQ ID NO: 60 - DNA, 18 bp, synthetic construct
  misc_feature 1..18: note = Description of Artificial Sequence: Synthetic oligonucleotide
  source 1..18: mol_type = other DNA
aatgctctggatggtttg

SEQ ID NO: 61 - protein, 6 aa, synthetic construct
  REGION 1..6: note = Description of Artificial Sequence: Synthetic peptide
NALDGL

SEQ ID NO: 62 - DNA, 42 bp, synthetic construct
  misc_feature 1..42: note = Description of Artificial Sequence: Synthetic oligonucleotide
  source 1..42: mol_type = other DNA
gctgtgagagatcctctttctggtggctacaataagctgatt

SEQ ID NO: 63 - protein, 16 aa, synthetic construct
  REGION 1..16: note = Description of Artificial Sequence: Synthetic peptide
CAVRDPLSGGYNKLIF

SEQ ID NO: 64 - DNA, 941 bp, synthetic construct
  misc_feature 1..941: note = Description of Artificial Sequence: Synthetic polynucleotide
  source 1..941: mol_type = other DNA
gagcactcaaattgaaacctgcctgatgtgggatgtgctgtggctgctgctttgttgcttgggacctcctctgacctaggatcagacacagagtctgagttctggggcctggaacctcaatgtgcacttgaacaatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgtattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 65 - DNA, 21 bp, synthetic construct
  misc_feature 1..21: note = Description of Artificial Sequence: Synthetic oligonucleotide
  source 1..21: mol_type = other DNA
acaatcagtggaactgattac

SEQ ID NO: 66 - protein, 7 aa, synthetic construct
  REGION 1..7: note = Description of Artificial Sequence: Synthetic peptide
TISGTDY

SEQ ID NO: 67 - DNA, 15 bp, synthetic construct
  misc_feature 1..15: note = Description of Artificial Sequence: Synthetic oligonucleotide
  source 1..15: mol_type = other DNA
ggtcttacaagcaat

SEQ ID NO: 68 - protein, 5 aa, synthetic construct
  REGION 1..5: note = Description of Artificial Sequence: Synthetic peptide
GLTSN

SEQ ID NO: 69 - DNA, 36 bp, synthetic construct
  misc_feature 1..36: note = Description of Artificial Sequence: Synthetic oligonucleotide
  source 1..36: mol_type = other DNA
atcctgtattcaggaggaggtgctgacggactcacc

SEQ ID NO: 70 - protein, 14 aa, synthetic construct
  REGION 1..14: note = Description of Artificial Sequence: Synthetic peptide
CILYSGGGADGLTF

SEQ ID NO: 71 - DNA, 1487 bp, synthetic construct
  misc_feature 1..1487: note = Description of Artificial Sequence: Synthetic polynucleotide
  source 1..1487: mol_type = other DNA
ttcctgtcttgagaagagaaacacttatggggaaattgaatactttgtcttttttttttttttttttgagacggagtctcactttgtcgcccaggctggagtgcagtggcgcgatctctgctcactgcaaactccgcctcccgggttcccgccattctcctgcctcagcctctcgagtagctgggactacaggcgcccgccacagcgcccagctaatttttttgtattttttggtagagacggggtttcaccgtgttagccaggatggtctcgatctcctgacctcgtgatccgcccacctcggcctcccaaagcgctgggattaaaggcgtgagccaccgcgcccggcctactttgtcttatgttattcccatttgccgtctctgttccttatacattatgctttttcaactttaccagaatcacttggattaaaacccgtggatttctcagtaggaaatgttcatgtgaagacacttctgtagtaacagaacctacagctgctcctgtagaaggaagttgaaagtcatcccttcaagaaaggggctcctccccttgtaattctactgggttttgcatccagactgagtttccttccctcacccacatgaagtgtctaccttctgcagactccaatggctcaggaactgggaatgcagtgccaggctcgtggtatcctgcagcagatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctggcctcttcggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 72 - DNA, 18 bp, synthetic construct
  misc_feature 1..18: note = Description of Artificial Sequence: Synthetic oligonucleotide
  source 1..18: mol_type = other DNA
acatctgggttcaacggg

SEQ ID NO: 73 - protein, 6 aa, synthetic construct
  REGION 1..6: note = Description of Artificial Sequence: Synthetic peptide
TSGFNG

SEQ ID NO: 74 - DNA, 18 bp, synthetic construct
  misc_feature 1..18: note = Description of Artificial Sequence: Synthetic oligonucleotide
  source 1..18: mol_type = other DNA
aatgttctggatggtttg

SEQ ID NO: 75 - protein, 6 aa, synthetic construct
  REGION 1..6: note = Description of Artificial Sequence: Synthetic peptide
NVLDGL

SEQ ID NO: 76 - DNA, 30 bp, synthetic construct
  misc_feature 1..30: note = Description of Artificial Sequence: Synthetic oligonucleotide
  source 1..30: mol_type = other DNA
gctggcctcttcggaaacacacctcttgtc

SEQ ID NO: 77 - protein, 12 aa, synthetic construct
  REGION 1..12: note = Description of Artificial Sequence: Synthetic peptide
CAGLFGNTPLVF

SEQ ID NO: 78 - DNA, 917 bp, synthetic construct
  misc_feature 1..917: note = Description of Artificial Sequence: Synthetic polynucleotide
  source 1..917: mol_type = other DNA
tggggctgttctgaagcagctacggcaccagtgcagctgatactcaaggttcagatcagaagaggaggcttctcaccctgcagcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttattttaacaccgacaagctcatctttgggactgggaccagattacaagtctttccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 79 - DNA, 21 bp, synthetic construct
  misc_feature 1..21: note = Description of Artificial Sequence: Synthetic oligonucleotide
  source 1..21: mol_type = other DNA
accagtgagagtgattattat

SEQ ID NO: 80 - protein, 7 aa, synthetic construct
  REGION 1..7: note = Description of Artificial Sequence: Synthetic peptide
TSESDYY

SEQ ID NO: 81 - DNA, 24 bp, synthetic construct
  misc_feature 1..24: note = Description of Artificial Sequence: Synthetic oligonucleotide
  source 1..24: mol_type = other DNA
caagaagcttataagcaacagaat

SEQ ID NO: 82 - protein, 8 aa, synthetic construct
  REGION 1..8: note = Description of Artificial Sequence: Synthetic peptide
QEAYKQQN

SEQ ID NO: 83 - DNA, 27 bp, synthetic construct
  misc_feature 1..27: note = Description of Artificial Sequence: Synthetic oligonucleotide
  source 1..27: mol_type = other DNA
gcttattttaacaccgacaagctcatc

SEQ ID NO: 84 - protein, 11 aa, synthetic construct
  REGION 1..11: note = Description of Artificial Sequence: Synthetic peptide
CAYFNTDKLIF

SEQ ID NO: 85 - DNA, 1035 bp, synthetic construct
  misc_feature 1..1035: note = Description of Artificial Sequence: Synthetic polynucleotide
  source 1..1035: mol_type = other DNA
ggcacacacacacatgaataagagcaagacagagggagagatgagggaggagcttaatgatggagcagaggtgttaaaaagaacatcctttttctaattggtaggacagatttcttttatgattcctacagcagaaaaatgagaaacgtttgttattatttttttttcgtgtttaaggtttgaatcctcagtgaaccagggcagaaaagaatgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatggggggccaaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 86 - DNA, 18 bp, synthetic construct
  misc_feature 1..18: note = Description of Artificial Sequence: Synthetic oligonucleotide
  source 1..18: mol_type = other DNA
aacagtgcttttcaatac

SEQ ID NO: 87 - protein, 6 aa, synthetic construct
  REGION 1..6: note = Description of Artificial Sequence: Synthetic peptide
NSAFQY

SEQ ID NO: 88 - DNA, 18 bp, synthetic construct
  misc_feature 1..18: note = Description of Artificial Sequence: Synthetic oligonucleotide
  source 1..18: mol_type = other DNA
acatactccagtggtaac

SEQ ID NO: 89 - protein, 6 aa, synthetic construct
  REGION 1..6: note = Description of Artificial Sequence: Synthetic peptide
TYSSGN

SEQ ID NO: 90 - DNA, 36 bp, synthetic construct
  misc_feature 1..36: note = Description of Artificial Sequence: Synthetic oligonucleotide
  source 1..36: mol_type = other DNA
gcaatggggggccaaggaggtgctgacggactcacc

SEQ ID NO: 91 - protein, 14 aa, synthetic construct
  REGION 1..14: note = Description of Artificial Sequence: Synthetic peptide
CAMGGQGGADGLTF

SEQ ID NO: 92 - DNA, 898 bp, synthetic construct
  misc_feature 1..898: note = Description of Artificial Sequence: Synthetic polynucleotide
  source 1..898: mol_type = other DNA
gggagtgtcactctaagcccaagagagtttcttgaagcaaaaaaaaaaaaaaacccattcaggaaataattctttgctgataaggatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggccccaagagggaatatggaaacaagctggtctttggcgcaggaaccattctgagagtcaagtcctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 93 - DNA, 15 bp, synthetic construct
  misc_feature 1..15: note = Description of Artificial Sequence: Synthetic oligonucleotide
  source 1..15: mol_type = other DNA
agcatatttaacacc

SEQ ID NO: 94 - protein, 5 aa, synthetic construct
  REGION 1..5: note = Description of Artificial Sequence: Synthetic peptide
SIFNT

SEQ ID NO: 95 - DNA, 21 bp, synthetic construct
  misc_feature 1..21: note = Description of Artificial Sequence: Synthetic oligonucleotide
  source 1..21: mol_type = other DNA
ttatataaggctggtgaattg

SEQ ID NO: 96 - protein, 7 aa, synthetic construct
  REGION 1..7: note = Description of Artificial Sequence: Synthetic peptide
LYKAGEL

SEQ ID NO: 97 - DNA, 36 bp, synthetic construct
  misc_feature 1..36: note = Description of Artificial Sequence: Synthetic oligonucleotide
  source 1..36: mol_type = other DNA
gctggccccaagagggaatatggaaacaagctggtc

SEQ ID NO: 98 - protein, 14 aa, synthetic construct
  REGION 1..14: note = Description of Artificial Sequence: Synthetic peptide
CAGPKREYGNKLVF

SEQ ID NO: 99 - DNA, 943 bp, synthetic construct
  misc_feature 1..943: note = Description of Artificial Sequence: Synthetic polynucleotide
  source 1..943: mol_type = other DNA
ggggacaactgtagcaacccttctaaaggttgtagattctggctgatgatgtcactgacacaaaggaaaaaatgcaaaacaggtagtcttaaataagcattctggtgagaccaactgcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggaggataacaataacaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 100 - DNA, 18 bp, synthetic construct
  misc_feature 1..18: note = Description of Artificial Sequence: Synthetic oligonucleotide
  source 1..18: mol_type = other DNA
gtgtccaatgcttacaac

SEQ ID NO: 101 - protein, 6 aa, synthetic construct
  REGION 1..6: note = Description of Artificial Sequence: Synthetic peptide
VSNAYN

SEQ ID NO: 102 - DNA, 12 bp, synthetic construct
  misc_feature 1..12: note = Description of Artificial Sequence: Synthetic oligonucleotide
  source 1..12: mol_type = other DNA
ggctcaaagcct

SEQ ID NO: 103 - protein, 4 aa, synthetic construct
  REGION 1..4: note = Description of Artificial Sequence: Synthetic peptide
GSKP

SEQ ID NO: 104 - DNA, 33 bp, synthetic construct
  misc_feature 1..33: note = Description of Artificial Sequence: Synthetic oligonucleotide
  source 1..33: mol_type = other DNA
gctgtggaggataacaataacaatgacatgcgc

SEQ ID NO: 105 - protein, 13 aa, synthetic construct
  REGION 1..13: note = Description of Artificial Sequence: Synthetic peptide
CAVEDNNNNDMRF

SEQ ID NO: 106 - DNA, 1277 bp, synthetic construct
  misc_feature 1..1277: note = Description of Artificial Sequence: Synthetic polynucleotide
  source 1..1277: mol_type = other DNA
tcaaattgactagttttgactttgccttatgttcccatttgttttctctgttctttacatgttcgatgttcaccataatcacttggattaaaatgtgtggattagtttttggagatagggacctcaccatgttgcttaggctggtctccagttcctggcctcaagggattcttctacctcagcgtcttgagtagctgggattacaggcataagccactgtgcccagcttaaaacctgtggatttatcagtagaaaatgttcatgtaaagatactcctgtaagagaaaccatagctgctccagtggaaggaagcttaaactcatcccttcaagaaagaagctcctccctttgtatttctactgggttttgcatccggactgatcttccttccctcacccacatgaagtgtctaccttctgcagactacagtggctcaggaaccggggatgcagtgccaggctcatggtatcctgcagcagatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgaataacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 107 - DNA, 18 bp, synthetic construct
  misc_feature 1..18: note = Description of Artificial Sequence: Synthetic oligonucleotide
  source 1..18: mol_type = other DNA
acatctgggttttatggg

SEQ ID NO: 108 - protein, 6 aa, synthetic construct
  REGION 1..6: note = Description of Artificial Sequence: Synthetic peptide
TSGFYG

SEQ ID NO: 109 - DNA, 18 bp, synthetic construct
  misc_feature 1..18: note = Description of Artificial Sequence: Synthetic oligonucleotide
  source 1..18: mol_type = other DNA
aatgctctggatggtttg

SEQ ID NO: 110 - protein, 6 aa, synthetic construct
  REGION 1..6: note = Description of Artificial Sequence: Synthetic peptide
NALDGL

SEQ ID NO: 111 - DNA, 27 bp, synthetic construct
  misc_feature 1..27: note = Description of Artificial Sequence: Synthetic oligonucleotide
  source 1..27: mol_type = other DNA
gctgtgaataacaatgccagactcatg

SEQ ID NO: 112 - protein, 11 aa, synthetic construct
  REGION 1..11: note = Description of Artificial Sequence: Synthetic peptide
CAVNNNARLMF

SEQ ID NO: 113 - DNA, 1479 bp, synthetic construct
  misc_feature 1..1479: note = Description of Artificial Sequence: Synthetic polynucleotide
  source 1..1479: mol_type = other DNA
tgtcttgagaagagaaacacttatggggaaattgaatacttttttttttttttttttttgagacggagtctcactttgtcgcccaggctggagtgcagtggcgcgatctctgctcactgcaaactccgcctcccgggttcccgccattctcctgcctcagcctctcgagtagctgggactacaggcgcccgccacagcgcccagctaatttttttgtattttttggtagagacggggtttcaccgtgttagccaggatggtctcgatctcctgacctcgtgatccgcccacctcggcctcccaaagcgctgggattaaaggcgtgagccaccgcgcccggcctactttgtcttatgttattcccatttgccgtctctgttccttatacattatgctttttcaactttaccagaatcacttggattaaaacccgtggatttctcagtaggaaatgttcatgtgaagacacttctgtagtaacagaacctacagctgctcctgtagaaggaagttgaaagtcatcccttcaagaaaggggctcctccccttgtaattctactgggttttgcatccagactgagtttccttccctcacccacatgaagtgtctaccttctgcagactccaatggctcaggaactgggaatgcagtgccaggctcgtggtatcctgcagcagatgtggggagttttccttctttatgtttccatgaagatgggaggcactacaggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagatccgggcaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 114 - DNA, 18 bp, synthetic construct
  misc_feature 1..18: note = Description of Artificial Sequence: Synthetic oligonucleotide
  source 1..18: mol_type = other DNA
acatctgggttcaacggg

SEQ ID NO: 115 - protein, 6 aa, synthetic construct
  REGION 1..6: note = Description of Artificial Sequence: Synthetic peptide
TSGFNG

SEQ ID NO: 116 - DNA, 18 bp, synthetic construct
  misc_feature 1..18: note = Description of Artificial Sequence: Synthetic oligonucleotide
  source 1..18: mol_type = other DNA
aatgttctggatggtttg

SEQ ID NO: 117 - protein, 6 aa, synthetic construct
  REGION 1..6: note = Description of Artificial Sequence: Synthetic peptide
NVLDGL

SEQ ID NO: 118 - DNA, 30 bp, synthetic construct
  misc_feature 1..30: note = Description of Artificial Sequence: Synthetic oligonucleotide
  source 1..30: mol_type = other DNA
gctgtgagagatccgggcaatgacatgcgc

SEQ ID NO: 119 - protein, 12 aa, synthetic construct
  REGION 1..12: note = Description of Artificial Sequence: Synthetic peptide
CAVRDPGNDMRF

SEQ ID NO: 120 - DNA, 938 bp, synthetic construct
  misc_feature 1..938: note = Description of Artificial Sequence: Synthetic polynucleotide
  source 1..938: mol_type = other DNA
gggacccacctatcatagcatttcctgccctgaaggagaattctcaccaagcacagaggagaacccatcagagcaggagacttttcactctgcaggggagcgctgtcagcatgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcgggggagaaaccagtggctctaggttgacctttggggaaggaacacagctcacagtgaatcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 121 - DNA, 21 bp, synthetic construct
  misc_feature 1..21: note = Description of Artificial Sequence: Synthetic oligonucleotide
  source 1..21: mol_type = other DNA
accagtgagaataattattat

SEQ ID NO: 122 - protein, 7 aa, synthetic construct
  REGION 1..7: note = Description of Artificial Sequence: Synthetic peptide
TSENNYY

SEQ ID NO: 123 - DNA, 24 bp, synthetic construct
  misc_feature 1..24: note = Description of Artificial Sequence: Synthetic oligonucleotide
  source 1..24: mol_type = other DNA
caagaagcttataagcaacagaat

SEQ ID NO: 124 - protein, 8 aa, synthetic construct
  REGION 1..8: note = Description of Artificial Sequence: Synthetic peptide
QEAYKQQN

SEQ ID NO: 125 - DNA, 36 bp, synthetic construct
  misc_feature 1..36: note = Description of Artificial Sequence: Synthetic oligonucleotide
  source 1..36: mol_type = other DNA
gctttcgggggagaaaccagtggctctaggttgacc

SEQ ID NO: 126 - protein, 14 aa, synthetic construct
  REGION 1..14: note = Description of Artificial Sequence: Synthetic peptide
CAFGGETSGSRLTF

SEQ ID NO: 127 - DNA, 905 bp, synthetic construct
  misc_feature 1..905: note = Description of Artificial Sequence: Synthetic polynucleotide
  source 1..905: mol_type = other DNA
agggatgtttttcttatatggggagttgctgctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgacaaaggaagcaactatcagttaatctggggcgctgggaccaagctaattataaagccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 128 - DNA, 21 bp, synthetic construct
  misc_feature 1..21: note = Description of Artificial Sequence: Synthetic oligonucleotide
  source 1..21: mol_type = other DNA
aacattgctacaaatgattat

SEQ ID NO: 129 - protein, 7 aa, synthetic construct
  REGION 1..7: note = Description of Artificial Sequence: Synthetic peptide
NIATNDY

SEQ ID NO: 130 - DNA, 15 bp, synthetic construct
  misc_feature 1..15: note = Description of Artificial Sequence: Synthetic oligonucleotide
  source 1..15: mol_type = other DNA
ggatacaagacaaaa

SEQ ID NO: 131 - protein, 5 aa, synthetic construct
  REGION 1..5: note = Description of Artificial Sequence: Synthetic peptide
GYKTK

SEQ ID NO: 132 - DNA, 36 bp, synthetic construct
  misc_feature 1..36: note = Description of Artificial Sequence: Synthetic oligonucleotide
  source 1..36: mol_type = other DNA
ctcgtgggtgacaaaggaagcaactatcagttaatc

SEQ ID NO: 133 - protein, 14 aa, synthetic construct
  REGION 1..14: note = Description of Artificial Sequence: Synthetic peptide
CLVGDKGSNYQLIW

SEQ ID NO: 134 - DNA, 1063 bp, synthetic construct
  misc_feature 1..1063: note = Description of Artificial Sequence: Synthetic polynucleotide
  source 1..1063: mol_type = other DNA
gaagttcagtagttagggatgtggccacaagatggcagtgctcctctgctgaggcagaatacagggttcactttagtgtgtcctgaatgaataggtttatggtagcagcagagcctttttcttattggttggctacacagtgtgagaaacccctatggctgccagaggagagaagagacaacctgatgatagaagtaactcttataactggaggttgcaggtcaatgactgatcttaattgggaagaacaaggatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagttatctccctgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 135 - DNA, 18 bp, synthetic construct
  misc_feature 1..18: note = Description of Artificial Sequence: Synthetic oligonucleotide
  source 1..18: mol_type = other DNA
gacagtgcctcaaactac

SEQ ID NO: 136 - protein, 6 aa, synthetic construct
  REGION 1..6: note = Description of Artificial Sequence: Synthetic peptide
DSASNY

SEQ ID NO: 137 - DNA, 21 bp, synthetic construct
  misc_feature 1..21: note = Description of Artificial Sequence: Synthetic oligonucleotide
  source 1..21: mol_type = other DNA
attcgttcaaatgtgggcgaa

SEQ ID NO: 138 - protein, 7 aa, synthetic construct
  REGION 1..7: note = Description of Artificial Sequence: Synthetic peptide
IRSNVGE

SEQ ID NO: 139 - DNA, 27 bp, synthetic construct
  misc_feature 1..27: note = Description of Artificial Sequence: Synthetic oligonucleotide
  source 1..27: mol_type = other DNA
gcagcaagttatctccctgacatgcgc

SEQ ID NO: 140 - protein, 11 aa, synthetic construct
  REGION 1..11: note = Description of Artificial Sequence: Synthetic peptide
CAASYLPDMRF

SEQ ID NO: 141 - DNA, 1036 bp, synthetic construct
  misc_feature 1..1036: note = Description of Artificial Sequence: Synthetic polynucleotide
  source 1..1036: mol_type = other DNA
gtctagcatcttccatctctacaggaagtatgtgacatgacacagtcaaactcatcatatatctgttaactatacttcctgtaaaaggcagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagctcaaagctgcaggcaacaagctaacttttggaggaggaaccagggtgctagttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 142 - DNA, 21 bp, synthetic construct
  misc_feature 1..21: note = Description of Artificial Sequence: Synthetic oligonucleotide
  source 1..21: mol_type = other DNA
acccgtgatactacttattac

SEQ ID NO: 143 - protein, 7 aa, synthetic construct
  REGION 1..7: note = Description of Artificial Sequence: Synthetic peptide
TRDTTYY

SEQ ID NO: 144 - DNA, 24 bp, synthetic construct
  misc_feature 1..24: note = Description of Artificial Sequence: Synthetic oligonucleotide
  source 1..24: mol_type = other DNA
cggaactcttttgatgagcaaaat

SEQ ID NO: 145 - protein, 8 aa, synthetic construct
  REGION 1..8: note = Description of Artificial Sequence: Synthetic peptide
RNSFDEQN

SEQ ID NO: 146 - DNA, 39 bp, synthetic construct
  misc_feature 1..39: note = Description of Artificial Sequence: Synthetic oligonucleotide
  source 1..39: mol_type = other DNA
gctctgagtgagctcaaagctgcaggcaacaagctaact

SEQ ID NO: 147 - protein, 15 aa, synthetic construct
  REGION 1..15: note = Description of Artificial Sequence: Synthetic peptide
CALSELKAAGNKLTF

SEQ ID NO: 148 - DNA, 939 bp, synthetic construct
  misc_feature 1..939: note = Description of Artificial Sequence: Synthetic polynucleotide
  source 1..939: mol_type = other DNA
ggctgtactggggcagcctgagtgacagctgctggtgtgggccctggcagttgctgctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgacaaggtttatggaggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 149 - DNA, 21 bp, synthetic construct
  misc_feature 1..21: note = Description of Artificial Sequence: Synthetic oligonucleotide
  source 1..21: mol_type = other DNA
aacattgctacaaatgattat

SEQ ID NO: 150 - protein, 7 aa, synthetic construct
  REGION 1..7: note = Description of Artificial Sequence: Synthetic peptide
NIATNDY

SEQ ID NO: 151 - DNA, 15 bp, synthetic construct
  misc_feature 1..15: note = Description of Artificial Sequence: Synthetic oligonucleotide
  source 1..15: mol_type = other DNA
ggatacaagacaaaa

SEQ ID NO: 152 - protein, 5 aa, synthetic construct
  REGION 1..5: note = Description of Artificial Sequence: Synthetic peptide
GYKTK

SEQ ID NO: 153 - DNA, 45 bp, synthetic construct
  misc_feature 1..45: note = Description of Artificial Sequence: Synthetic oligonucleotide
  source 1..45: mol_type = other DNA
ctcgtgggtgacaaggtttatggaggaagccaaggaaatctcatc

SEQ ID NO: 154 - protein, 17 aa, synthetic construct
  REGION 1..17: note = Description of Artificial Sequence: Synthetic peptide
CLVGDKVYGGSQGNLIF

SEQ ID NO: 155 - DNA, 1074 bp, synthetic construct
  misc_feature 1..1074: note = Description of Artificial Sequence: Synthetic polynucleotide
  source 1..1074: mol_type = other DNA
ggggagttggttgatccaaaagaaagtctgcattgggtgagcttctcagccagtacatagagaagtgtgagatgacacacactagtttgctataccaggaaacctctgtttcctgttctagggcagcagtagcagcagcacatggcccagtaattcttctctcaccatgccaggttcacttcacagtacagagtcctgaaaataaagaagaaaatttttttttatctagaaaaagaaccaaacatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtatttctgtgcaatgagagagggccaggatagcagctataaattgatcttcgggagtgggaccagactgctggtcaggcctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 156 - DNA, 21 bp, synthetic construct
  misc_feature 1..21: note = Description of Artificial Sequence: Synthetic oligonucleotide
  source 1..21: mol_type = other DNA
accagtgatccaagttatggt

SEQ ID NO: 157 - protein, 7 aa, synthetic construct
  REGION 1..7: note = Description of Artificial Sequence: Synthetic peptide
TSDPSYG

SEQ ID NO: 158 - DNA, 24 bp, synthetic construct
  misc_feature 1..24: note = Description of Artificial Sequence: Synthetic oligonucleotide
  source 1..24: mol_type = other DNA
caggggtcttatgaccagcaaaat

SEQ ID NO: 159 - protein, 8 aa, synthetic construct
  REGION 1..8: note = Description of Artificial Sequence: Synthetic peptide
QGSYDQQN

SEQ ID NO: 160 - DNA, 39 bp, synthetic construct
  misc_feature 1..39: note = Description of Artificial Sequence: Synthetic oligonucleotide
  source 1..39: mol_type = other DNA
gcaatgagagagggccaggatagcagctataaattgatc

SEQ ID NO: 161 - protein, 15 aa, synthetic construct
  REGION 1..15: note = Description of Artificial Sequence: Synthetic peptide
CAMREGQDSSYKLIF

SEQ ID NO: 162 - DNA, 964 bp, synthetic construct
  misc_feature 1..964: note = Description of Artificial Sequence: Synthetic polynucleotide
  source 1..964: mol_type = other DNA
tctggggttcatatgtaaaatgaagggtctgtggaaggacatgaataaagcacaggaggttgaagtcagatttgcagctttctaggcaggagacaagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaaggatttatggaggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 163 - DNA, 18 bp, synthetic construct
  misc_feature 1..18: note = Description of Artificial Sequence: Synthetic oligonucleotide
  source 1..18: mol_type = other DNA
aacagcatgtttgattat

SEQ ID NO: 164 - protein, 6 aa, synthetic construct
  REGION 1..6: note = Description of Artificial Sequence: Synthetic peptide
NSMFDY

SEQ ID NO: 165 - DNA, 21 bp, synthetic construct
  misc_feature 1..21: note = Description of Artificial Sequence: Synthetic oligonucleotide
  source 1..21: mol_type = other DNA
ataagttccattaaggataaa

SEQ ID NO: 166 - protein, 7 aa, synthetic construct
  REGION 1..7: note = Description of Artificial Sequence: Synthetic peptide
ISSIKDK

SEQ ID NO: 167 - DNA, 39 bp, synthetic construct
  misc_feature 1..39: note = Description of Artificial Sequence: Synthetic oligonucleotide
  source 1..39: mol_type = other DNA
gcagcaaggatttatggaggaagccaaggaaatctcatc

SEQ ID NO: 168 - protein, 15 aa, synthetic construct
  REGION 1..15: note = Description of Artificial Sequence: Synthetic peptide
CAARIYGGSQGNLIF

SEQ ID NO: 169 - DNA, 1059 bp, synthetic construct
  misc_feature 1..1059: note = Description of Artificial Sequence: Synthetic polynucleotide
  source 1..1059: mol_type = other DNA
gaaagtctgcattgggtgagcttctcagccagtacatagagaagtgtgagatgacacacactagtttgctataccaggaaacctctgtttcctgttctagggcagcagtagcagcagcacatggcccagtaattcttctctcaccatgccaggttcacttcacagtacagagtcctgaaaataaagaagaaaatttttttttatctagaaaaagaaccaaacatgtcactttctagcctgctgaaggtggtcacagcttcactgtggctaggacctggcattgcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagagggcgaaccttctggttctgcaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 170 - DNA, 21 bp, synthetic construct
  misc_feature 1..21: note = Description of Artificial Sequence: Synthetic oligonucleotide
  source 1..21: mol_type = other DNA
accagtgatccaagttatggt

SEQ ID NO: 171 - protein, 7 aa, synthetic construct
  REGION 1..7: note = Description of Artificial Sequence: Synthetic peptide
TSDPSYG

SEQ ID NO: 172 - DNA, 24 bp, synthetic construct
  misc_feature 1..24: note = Description of Artificial Sequence: Synthetic oligonucleotide
  source 1..24: mol_type = other DNA
caggggtcttatgaccagcaaaat

SEQ ID NO: 173 - protein, 8 aa, synthetic construct
  REGION 1..8: note = Description of Artificial Sequence: Synthetic peptide
QGSYDQQN

SEQ ID NO: 174 - DNA, 45 bp, synthetic construct
  misc_feature 1..45: note = Description of Artificial Sequence: Synthetic oligonucleotide
  source 1..45: mol_type = other DNA
gcaatgagagagggcgaaccttctggttctgcaaggcaactgacc

SEQ ID NO: 175 - protein, 17 aa, synthetic construct
  REGION 1..17: note = Description of Artificial Sequence: Synthetic peptide
CAMREGEPSGSARQLTF

SEQ ID NO: 176 - DNA, 962 bp, synthetic construct
  misc_feature 1..962: note = Description of Artificial Sequence: Synthetic polynucleotide
  source 1..962: mol_type = other DNA
tggggttcatatgtaaaatgaagggtctgtggaaggacatgaataaagcacaggaggttgaagtcagatttgcagctttctaggcaggagacaagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagttaatgctggtggtactagctatggaaagctgacatttggacaagggaccatcttgactgtccatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 177 - DNA, 18 bp, synthetic construct
  misc_feature 1..18: note = Description of Artificial Sequence: Synthetic oligonucleotide
  source 1..18: mol_type = other DNA
aacagcatgtttgattat

SEQ ID NO: 178 - protein, 6 aa, synthetic construct
  REGION 1..6: note = Description of Artificial Sequence: Synthetic peptide
NSMFDY

SEQ ID NO: 179 - DNA, 21 bp, synthetic construct
  misc_feature 1..21: note = Description of Artificial Sequence: Synthetic oligonucleotide
  source 1..21: mol_type = other DNA
ataagttccattaaggataaa

SEQ ID NO: 180 - protein, 7 aa, synthetic construct
  REGION 1..7: note = Description of Artificial Sequence: Synthetic peptide
ISSIKDK

SEQ ID NO: 181 - DNA, 39 bp, synthetic construct
  misc_feature 1..39: note = Description of Artificial Sequence: Synthetic oligonucleotide
  source 1..39: mol_type = other DNA
gcagttaatgctggtggtactagctatggaaagctgaca

SEQ ID NO: 182 - protein, 15 aa, synthetic construct
  REGION 1..15: note = Description of Artificial Sequence: Synthetic peptide
CAVNAGGTSYGKLTF

SEQ ID NO: 183 - DNA, 1202 bp, synthetic construct
  misc_feature 1..1202: note = Description of Artificial Sequence: Synthetic polynucleotide
  source 1..1202: mol_type = other DNA
gacatttctcaaatgagaagcaaacagttcacttccttggatccgtggtttcgcctgtggccttcagggggcgacgttgcactaaggaggcatctgtgttcattgccgaccatcctcatccactgagcctcctccctgcagctggctgatgtagctcactggtgtctgtgtagatagggagctgtgatgagaacaagaggtcagaacacatccaggctccttaagagaaagcctttctttaaccatttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgagggagccctggttctgctgaggtgcaactactcatcgtctgttccaccatatctcttctggtatgtgcaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgagtgagaagttttctggtggctacaataagctgatttttggagcagggaccaggctggctgtacacccatatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 184 - DNA, 18 bp, synthetic construct
  misc_feature 1..18: note = Description of Artificial Sequence: Synthetic oligonucleotide
  source 1..18: mol_type = other DNA
tcgtctgttccaccatat

SEQ ID NO: 185 - protein, 6 aa, synthetic construct
  REGION 1..6: note = Description of Artificial Sequence: Synthetic peptide
SSVPPY

SEQ ID NO: 186 - DNA, 24 bp, synthetic construct
  misc_feature 1..24: note = Description of Artificial Sequence: Synthetic oligonucleotide
  source 1..24: mol_type = other DNA
tacacaacaggggccaccctggtt

SEQ ID NO: 187 - protein, 8 aa, synthetic construct
  REGION 1..8: note = Description of Artificial Sequence: Synthetic peptide
YTTGATLV

SEQ ID NO: 188 - DNA, 42 bp, synthetic construct
  misc_feature 1..42: note = Description of Artificial Sequence: Synthetic oligonucleotide
  source 1..42: mol_type = other DNA
gctgtgagtgagaagttttctggtggctacaataagctgatt

SEQ ID NO: 189 - protein, 16 aa, synthetic construct
  REGION 1..16: note = Description of Artificial Sequence: Synthetic peptide
CAVSEKFSGGYNKLIF

SEQ ID NO: 190 - DNA, 971 bp, synthetic construct
  misc_feature 1..971: note = Description of Artificial Sequence: Synthetic polynucleotide
  source 1..971: mol_type = other DNA
tgggggttaactatacttcctgtaaaaggcagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcaaaagatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 191 - DNA, 21 bp, synthetic construct
  misc_feature 1..21: note = Description of Artificial Sequence: Synthetic oligonucleotide
  source 1..21: mol_type = other DNA
acccgtgatactacttattac

SEQ ID NO: 192 - protein, 7 aa, synthetic construct
  REGION 1..7: note = Description of Artificial Sequence: Synthetic peptide
TRDTTYY

SEQ ID NO: 193 - DNA, 24 bp, synthetic construct
  misc_feature 1..24: note = Description of Artificial Sequence: Synthetic oligonucleotide
  source 1..24: mol_type = other DNA
cggaactcttttgatgagcaaaat

SEQ ID NO: 194 - protein, 8 aa, synthetic construct
  REGION 1..8: note = Description of Artificial Sequence: Synthetic peptide
RNSFDEQN

SEQ ID NO: 195 - DNA, 33 bp, synthetic construct
  misc_feature 1..33: note = Description of Artificial Sequence: Synthetic oligonucleotide
  source 1..33: mol_type = other DNA
gctctgagtgaggcaaaagatgacaagatcatc

SEQ ID NO: 196 - protein, 13 aa, synthetic construct
  REGION 1..13: note = Description of Artificial Sequence: Synthetic peptide
CALSEAKDDKIIF

SEQ ID NO: 197 - DNA, 1008 bp, synthetic construct
  misc_feature 1..1008: note = Description of Artificial Sequence: Synthetic polynucleotide
  source 1..1008: mol_type = other DNA
ctcctatatcggtttttcttatatgggagaatctccatggcagcgttttcttatatggggttcatatgtaaaatgaagggtctgtggaaggacatgaataaagcacaggaggttgaagtcagatttgcagctttctaggcaggagacaagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgccatttataaccagggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 198 - DNA, 18 bp, synthetic construct
  misc_feature 1..18: note = Description of Artificial Sequence: Synthetic oligonucleotide
  source 1..18: mol_type = other DNA
aacagcatgtttgattat

SEQ ID NO: 199 - protein, 6 aa, synthetic construct
  REGION 1..6: note = Description of Artificial Sequence: Synthetic peptide
NSMFDY

SEQ ID NO: 200 - DNA, 21 bp, synthetic construct
  misc_feature 1..21: note = Description of Artificial Sequence: Synthetic oligonucleotide
  source 1..21: mol_type = other DNA
ataagttccattaaggataaa

SEQ ID NO: 201 - protein, 7 aa, synthetic construct
  REGION 1..7: note = Description of Artificial Sequence: Synthetic peptide
ISSIKDK

SEQ ID NO: 202 - DNA, 30 bp, synthetic construct
  misc_feature 1..30: note = Description of Artificial Sequence: Synthetic oligonucleotide
  source 1..30: mol_type = other DNA
gccatttataaccagggaggaaagcttatc

SEQ ID NO: 203 - protein, 12 aa, synthetic construct
  REGION 1..12: note = Description of Artificial Sequence: Synthetic peptide
CAIYNQGGKLIF

SEQ ID NO: 204 - DNA, 890 bp, synthetic construct
  misc_feature 1..890: note = Description of Artificial Sequence: Synthetic polynucleotide
  source 1..890: mol_type = other DNA
tacattctcaatttcttatatggggactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgatgaccgactacaagctcagctttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 205 - DNA, 18 bp, synthetic construct
  misc_feature 1..18: note = Description of Artificial Sequence: Synthetic oligonucleotide
  source 1..18: mol_type = other DNA
gccacaggatacccttcc

SEQ ID NO: 206 - protein, 6 aa, synthetic construct
  REGION 1..6: note = Description of Artificial Sequence: Synthetic peptide
ATGYPS

SEQ ID NO: 207 - DNA, 21 bp, synthetic construct
  misc_feature 1..21: note = Description of Artificial Sequence: Synthetic oligonucleotide
  source 1..21: mol_type = other DNA
gccacgaaggctgatgacaag

SEQ ID NO: 208 - protein, 7 aa, synthetic construct
  REGION 1..7: note = Description of Artificial Sequence: Synthetic peptide
ATKADDK

SEQ ID NO: 209 - DNA, 27 bp, synthetic construct
  misc_feature 1..27: note = Description of Artificial Sequence: Synthetic oligonucleotide
  source 1..27: mol_type = other DNA
gctctgatgaccgactacaagctcagc

SEQ ID NO: 210 - protein, 11 aa, synthetic construct
  REGION 1..11: note = Description of Artificial Sequence: Synthetic peptide
CALMTDYKLSF

SEQ ID NO: 211 - DNA, 987 bp, synthetic construct
  misc_feature 1..987: note = Description of Artificial Sequence: Synthetic polynucleotide
  source 1..987: mol_type = other DNA
gggagtggtgaattaggggtgttaaaaagagcatcatttttttgaactggtaaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaggagtgtaggggtttctggtggctacaataagctgatttttggagcagggaccaggctggctgtacacccatatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 212 - DNA, 18 bp, synthetic construct
  misc_feature 1..18: note = Description of Artificial Sequence: Synthetic oligonucleotide
  source 1..18: mol_type = other DNA
gaccgaggttcccagtcc

SEQ ID NO: 213 - protein, 6 aa, synthetic construct
  REGION 1..6: note = Description of Artificial Sequence: Synthetic peptide
DRGSQS

SEQ ID NO: 214 - DNA, 18 bp, synthetic construct
  misc_feature 1..18: note = Description of Artificial Sequence: Synthetic oligonucleotide
  source 1..18: mol_type = other DNA
atatactccaatggtgac

SEQ ID NO: 215 - protein, 6 aa, synthetic construct
  REGION 1..6: note = Description of Artificial Sequence: Synthetic peptide
IYSNGD

SEQ ID NO: 216 - DNA, 45 bp, synthetic construct
  misc_feature 1..45: note = Description of Artificial Sequence: Synthetic oligonucleotide
  source 1..45: mol_type = other DNA
gccgtgaggagtgtaggggtttctggtggctacaataagctgatt

SEQ ID NO: 217 - protein, 17 aa, synthetic construct
  REGION 1..17: note = Description of Artificial Sequence: Synthetic peptide
CAVRSVGVSGGYNKLIF

SEQ ID NO: 218 - DNA, 911 bp, synthetic construct
  misc_feature 1..911: note = Description of Artificial Sequence: Synthetic polynucleotide
  source 1..911: mol_type = other DNA
gggtgacagctgctggtgtgggccctggcagttgctgctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgttctactctctagcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 219 - DNA, 21 bp, synthetic construct
  misc_feature 1..21: note = Description of Artificial Sequence: Synthetic oligonucleotide
  source 1..21: mol_type = other DNA
aacattgctacaaatgattat

SEQ ID NO: 220 - protein, 7 aa, synthetic construct
  REGION 1..7: note = Description of Artificial Sequence: Synthetic peptide
NIATNDY

SEQ ID NO: 221 - DNA, 15 bp, synthetic construct
  misc_feature 1..15: note = Description of Artificial Sequence: Synthetic oligonucleotide
  source 1..15: mol_type = other DNA
ggatacaagacaaaa

SEQ ID NO: 222 - protein, 5 aa, synthetic construct
  REGION 1..5: note = Description of Artificial Sequence: Synthetic peptide
GYKTK

SEQ ID NO: 223 - DNA, 36 bp, synthetic construct
  misc_feature 1..36: note = Description of Artificial Sequence: Synthetic oligonucleotide
  source 1..36: mol_type = other DNA
ctcgttctactctctagcaacacaggcaaactaatc

SEQ ID NO: 224 - protein, 14 aa, synthetic construct
  REGION 1..14: note = Description of Artificial Sequence: Synthetic peptide
CLVLLSSNTGKLIF

SEQ ID NO: 225 - DNA, 923 bp, synthetic construct
  misc_feature 1..923: note = Description of Artificial Sequence: Synthetic polynucleotide
  source 1..923: mol_type = other DNA
tggggctgttctgaagcagctacggcaccagtgcagctgatactcaaggttcagatcagaagaggaggcttctcaccctgcagcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgcgaataacaatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 226 - DNA, 21 bp, synthetic construct
  misc_feature 1..21: note = Description of Artificial Sequence: Synthetic oligonucleotide
  source 1..21: mol_type = other DNA
accagtgagagtgattattat

SEQ ID NO: 227 - protein, 7 aa, synthetic construct
  REGION 1..7: note = Description of Artificial Sequence: Synthetic peptide
TSESDYY

SEQ ID NO: 228 - DNA, 24 bp, synthetic construct
  misc_feature 1..24: note = Description of Artificial Sequence: Synthetic oligonucleotide
  source 1..24: mol_type = other DNA
caagaagcttataagcaacagaat

SEQ ID NO: 229 - protein, 8 aa, synthetic construct
  REGION 1..8: note = Description of Artificial Sequence: Synthetic peptide
QEAYKQQN

SEQ ID NO: 230 - DNA, 33 bp, synthetic construct
  misc_feature 1..33: note = Description of Artificial Sequence: Synthetic oligonucleotide
  source 1..33: mol_type = other DNA
gcttataggagcgcgaataacaatgacatgcgc

SEQ ID NO: 231 - protein, 13 aa, synthetic construct
  REGION 1..13: note = Description of Artificial Sequence: Synthetic peptide
CAYRSANNNDMRF

SEQ ID NO: 232 - DNA, 1054 bp, synthetic construct
  misc_feature 1..1054: note = Description of Artificial Sequence: Synthetic polynucleotide
  source 1..1054: mol_type = other DNA
gggggtgtgtgtgtgtgcttgagagagagagaaggagagaaagagagagaatgggaagggcgataagaggaggggttagtgatataccaggggttgtgaaaataacctcttttttctaattggtaggacagattctttttatgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggttttcccaggaggaagctacatacctacatttggaagaggaaccagccttattgttcatccgtatatccagaagcctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 233 - DNA, 18 bp, synthetic construct
  misc_feature 1..18: note = Description of Artificial Sequence: Synthetic oligonucleotide
  source 1..18: mol_type = other DNA
aacagtgcttctcagtct

SEQ ID NO: 234 - protein, 6 aa, synthetic construct
  REGION 1..6: note = Description of Artificial Sequence: Synthetic peptide
NSASQS

SEQ ID NO: 235 - DNA, 18 bp, synthetic construct
  misc_feature 1..18: note = Description of Artificial Sequence: Synthetic oligonucleotide
  source 1..18: mol_type = other DNA
gtatactccagtggtaat

SEQ ID NO: 236 - protein, 6 aa, synthetic construct
  REGION 1..6: note = Description of Artificial Sequence: Synthetic peptide
VYSSGN

SEQ ID NO: 237 - DNA, 33 bp, synthetic construct
  misc_feature 1..33: note = Description of Artificial Sequence: Synthetic oligonucleotide
  source 1..33: mol_type = other DNA
gtggttttcccaggaggaagctacatacctaca

SEQ ID NO: 238 - protein, 13 aa, synthetic construct
  REGION 1..13: note = Description of Artificial Sequence: Synthetic peptide
CVVFPGGSYIPTF

SEQ ID NO: 239 - DNA, 1262 bp, synthetic construct
  misc_feature 1..1262: note = Description of Artificial Sequence: Synthetic polynucleotide
  source 1..1262: mol_type = other DNA
agaaagcagaatgggtgatgtgatgtgcaatgccacagaagcactgcagccaggagaggtgacagctaatggggatgtttggagtctttgagtgaaccaaacacatcccagagtaattgtaatttatttcagtcaatcttctgtacagacttagcattcacctttggaggaaggtcctttgagcagggacagagatggtgatgtcactgacagtcccccttttactctgggtgagaggtctagaatcctcagctcctgtattcgtgcccacaagggcctcatctaggtgaaggctccacctgccccaccctgccatggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggtttggccctcactgaggggggctggtacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 240 - DNA, 15 bp, synthetic construct
  misc_feature 1..15: note = Description of Artificial Sequence: Synthetic oligonucleotide
  source 1..15: mol_type = other DNA
ttaggccacaatact

SEQ ID NO: 241 - protein, 5 aa, synthetic construct
  REGION 1..5: note = Description of Artificial Sequence: Synthetic peptide
LGHNT

SEQ ID NO: 242 - DNA, 18 bp, synthetic construct
  misc_feature 1..18: note = Description of Artificial Sequence: Synthetic oligonucleotide
  source 1..18: mol_type = other DNA
ttccgcaaccgggctcct

SEQ ID NO: 243 - protein, 6 aa, synthetic construct
  REGION 1..6: note = Description of Artificial Sequence: Synthetic peptide
FRNRAP

SEQ ID NO: 244 - DNA, 45 bp, synthetic construct
  misc_feature 1..45: note = Description of Artificial Sequence: Synthetic oligonucleotide
  source 1..45: mol_type = other DNA
gctagtggtttggccctcactgaggggggctggtacgagcagtac

SEQ ID NO: 245 - protein, 15 aa, synthetic construct
  REGION 1..15: note = Description of Artificial Sequence: Synthetic peptide
ASGLALTEGGWYEQY

SEQ ID NO: 246 - DNA, 1068 bp, synthetic construct
  misc_feature 1..1068: note = Description of Artificial Sequence: Synthetic polynucleotide
  source 1..1068: mol_type = other DNA
gggccagagacaccagtaattctgccagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcctccgatgggacggggacaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 247 - DNA, 15 bp, synthetic construct
  misc_feature 1..15: note = Description of Artificial Sequence: Synthetic oligonucleotide
  source 1..15: mol_type = other DNA
tctaatcacttatac

SEQ ID NO: 248 - protein, 5 aa, synthetic construct
  REGION 1..5: note = Description of Artificial Sequence: Synthetic peptide
SNHLY

SEQ ID NO: 249 - DNA, 18 bp, synthetic construct
  misc_feature 1..18: note = Description of Artificial Sequence: Synthetic oligonucleotide
  source 1..18: mol_type = other DNA
ttttataataatgaaatc

SEQ ID NO: 250 - protein, 6 aa, synthetic construct
  REGION 1..6: note = Description of Artificial Sequence: Synthetic peptide
FYNNEI

SEQ ID NO: 251 - DNA, 36 bp, synthetic construct
  misc_feature 1..36: note = Description of Artificial Sequence: Synthetic oligonucleotide
  source 1..36: mol_type = other DNA
gccagcctccgatgggacggggacaatgagcagttc

SEQ ID NO: 252 - protein, 12 aa, synthetic construct
  REGION 1..12: note = Description of Artificial Sequence: Synthetic peptide
ASLRWDGDNEQF

SEQ ID NO: 253 - DNA, 1089 bp, synthetic construct
  misc_feature 1..1089: note = Description of Artificial Sequence: Synthetic polynucleotide
  source 1..1089: mol_type = other DNA
gaatttgaccatctggggaaggggcgtggcctctcctgacaggaaggctctggggcccaggcagggagaatgaagtctcagaatgacccccttgagagtactgttcccctatcaccgatgcacagacccagaagacccctccatcctgtagcacctgccatgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggacagggccctgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 254 - DNA, 15 bp, synthetic construct
  misc_feature 1..15: note = Description of Artificial Sequence: Synthetic oligonucleotide
  source 1..15: mol_type = other DNA
atgaaccataactcc

SEQ ID NO: 255 - protein, 5 aa, synthetic construct
  REGION 1..5: note = Description of Artificial Sequence: Synthetic peptide
MNHNS

SEQ ID NO: 256 - DNA, 18 bp, synthetic construct
  misc_feature 1..18: note = Description of Artificial Sequence: Synthetic oligonucleotide
  source 1..18: mol_type = other DNA
tcagcttctgagggtacc

SEQ ID NO: 257 - protein, 6 aa, synthetic construct
  REGION 1..6: note = Description of Artificial Sequence: Synthetic peptide
SASEGT

SEQ ID NO: 258 - DNA, 36 bp, synthetic construct
  misc_feature 1..36: note = Description of Artificial Sequence: Synthetic oligonucleotide
  source 1..36: mol_type = other DNA
gccagcagggacagggccctgaacactgaagctttc

SEQ ID NO: 259 - protein, 12 aa, synthetic construct
  REGION 1..12: note = Description of Artificial Sequence: Synthetic peptide
ASRDRALNTEAF

SEQ ID NO: 260 - DNA, 1258 bp, synthetic construct
  misc_feature 1..1258: note = Description of Artificial Sequence: Synthetic polynucleotide
  source 1..1258: mol_type = other DNA
gaagaagtctcatctgtcagtgagttgacaagaaacagagcaaaacgactcctccaatgttgacgagcctgcccctgggatttggaaacttcataacagaaaaaaccaatatagacaaaggattttaaacaggattaagctgttcactggtgcatttattttggatttgaccatctggggaatgggtgtggcctctcctggcctctccctccctggggcccaggcagggagaatgtctcagaatgacttccttgagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagaaacggcgggacactaatctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 261 - DNA, 15 bp, synthetic construct
  misc_feature 1..15: note = Description of Artificial Sequence: Synthetic oligonucleotide
  source 1..15: mol_type = other DNA
atgaaccatgaatac

SEQ ID NO: 262 - protein, 5 aa, synthetic construct
  REGION 1..5: note = Description of Artificial Sequence: Synthetic peptide
MNHEY

SEQ ID NO: 263 - DNA, 18 bp, synthetic construct
  misc_feature 1..18: note = Description of Artificial Sequence: Synthetic oligonucleotide
  source 1..18: mol_type = other DNA
tcagttggtgctggtatc

SEQ ID NO: 264 - protein, 6 aa, synthetic construct
  REGION 1..6: note = Description of Artificial Sequence: Synthetic peptide
SVGAGI

SEQ ID NO: 265 - DNA, 39 bp, synthetic construct
  misc_feature 1..39: note = Description of Artificial Sequence: Synthetic oligonucleotide
  source 1..39: mol_type = other DNA
gccagcagaaacggcgggacactaatctacgagcagtac

SEQ ID NO: 266 - protein, 13 aa, synthetic construct
  REGION 1..13: note = Description of Artificial Sequence: Synthetic peptide
ASRNGGTLIYEQY

SEQ ID NO: 267 - DNA, 1262 bp, synthetic construct
  misc_feature 1..1262: note = Description of Artificial Sequence: Synthetic polynucleotide
  source 1..1262: mol_type = other DNA
tgggggcaatgcccaaaaccccagctctcagaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcaggcagcggacagaacttgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 268 - DNA, 15 bp, synthetic construct
  misc_feature 1..15: note = Description of Artificial Sequence: Synthetic oligonucleotide
  source 1..15: mol_type = other DNA
tctgggcataggagt

SEQ ID NO: 269 - protein, 5 aa, synthetic construct
  REGION 1..5: note = Description of Artificial Sequence: Synthetic peptide
SGHRS

SEQ ID NO: 270 - DNA, 18 bp, synthetic construct
  misc_feature 1..18: note = Description of Artificial Sequence: Synthetic oligonucleotide
  source 1..18: mol_type = other DNA
tacttcagtgagacacag

SEQ ID NO: 271 - protein, 6 aa, synthetic construct
  REGION 1..6: note = Description of Artificial Sequence: Synthetic peptide
YFSETQ

SEQ ID NO: 272 - DNA, 33 bp, synthetic construct
  misc_feature 1..33: note = Description of Artificial Sequence: Synthetic oligonucleotide
  source 1..33: mol_type = other DNA
gccagcaggcagcggacagaacttgaagctttc

SEQ ID NO: 273 - protein, 11 aa, synthetic construct
  REGION 1..11: note = Description of Artificial Sequence: Synthetic peptide
ASRQRTELEAF

SEQ ID NO: 274 - DNA, 1073 bp, synthetic construct
  misc_feature 1..1073: note = Description of Artificial Sequence: Synthetic polynucleotide
  source 1..1073: mol_type = other DNA
ggccagagacaccagtaattctgccagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagcccctgacaggggtacctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 275 - DNA, 15 bp, synthetic construct
  misc_feature 1..15: note = Description of Artificial Sequence: Synthetic oligonucleotide
  source 1..15: mol_type = other DNA
tctaatcacttatac

SEQ ID NO: 276 - protein, 5 aa, synthetic construct
  REGION 1..5: note = Description of Artificial Sequence: Synthetic peptide
SNHLY

SEQ ID NO: 277 - DNA, 18 bp, synthetic construct
  misc_feature 1..18: note = Description of Artificial Sequence: Synthetic oligonucleotide
  source 1..18: mol_type = other DNA
ttttataataatgaaatc

SEQ ID NO: 278 - protein, 6 aa, synthetic construct
  REGION 1..6: note = Description of Artificial Sequence: Synthetic peptide
FYNNEI

SEQ ID NO: 279 - DNA, 42 bp, synthetic construct
  misc_feature 1..42: note = Description of Artificial Sequence: Synthetic oligonucleotide
  source 1..42: mol_type = other DNA
gccagcagtgaagcccctgacaggggtacctacgagcagtac

SEQ ID NO: 280 - protein, 14 aa, synthetic construct
  REGION 1..14: note = Description of Artificial Sequence: Synthetic peptide
ASSEAPDRGTYEQY

SEQ ID NO: 281 - DNA, 1011 bp, synthetic construct
  misc_feature 1..1011: note = Description of Artificial Sequence: Synthetic polynucleotide
  source 1..1011: mol_type = other DNA
ggggactgagagctcaacttcaatttgcccacagcagggctgggagacacaagatcctgccctggagctgaaatgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctacgttgtctccagatccaagacagagaatttccccctcactctggagtcagctacccgctcccagacatctgtgtatttctgcgccagcagtgagttgggcaggggtttctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 282 - DNA, 15 bp, synthetic construct
  misc_feature 1..15: note = Description of Artificial Sequence: Synthetic oligonucleotide
  source 1..15: mol_type = other DNA
tggagccacagctat

SEQ ID NO: 283 - protein, 5 aa, synthetic construct
  REGION 1..5: note = Description of Artificial Sequence: Synthetic peptide
WSHSY

SEQ ID NO: 284 - DNA, 18 bp, synthetic construct
  misc_feature 1..18: note = Description of Artificial Sequence: Synthetic oligonucleotide
  source 1..18: mol_type = other DNA
tcagcagctgctgatatt

SEQ ID NO: 285 - protein, 6 aa, synthetic construct
  REGION 1..6: note = Description of Artificial Sequence: Synthetic peptide
SAAADI

SEQ ID NO: 286 - DNA, 39 bp, synthetic construct
  misc_feature 1..39: note = Description of Artificial Sequence: Synthetic oligonucleotide
  source 1..39: mol_type = other DNA
gccagcagtgagttgggcaggggtttctacgagcagtac

SEQ ID NO: 287 - protein, 13 aa, synthetic construct
  REGION 1..13: note = Description of Artificial Sequence: Synthetic peptide
ASSELGRGFYEQY

SEQ ID NO: 288 - DNA, 1191 bp, synthetic construct
  misc_feature 1..1191: note = Description of Artificial Sequence: Synthetic polynucleotide
  source 1..1191: mol_type = other DNA
gatggaaagcaagagccctgggtggagctgaaggtgctcagctgggtttgtcagaagtctcatctgtcactgttcactggtgcatttattttggatttgaccatctggggaatgggtgtggcctctcctggcctctccctccctggggcccaggcagggagaatgtctcagaatgacttccttgagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagccatctgggggcgggagggccgcacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 289 - DNA, 15 bp, synthetic construct
  misc_feature 1..15: note = Description of Artificial Sequence: Synthetic oligonucleotide
  source 1..15: mol_type = other DNA
atgaaccatgaatac

SEQ ID NO: 290 - protein, 5 aa, synthetic construct
  REGION 1..5: note = Description of Artificial Sequence: Synthetic peptide
MNHEY

SEQ ID NO: 291 - DNA, 18 bp, synthetic construct
  misc_feature 1..18: note = Description of Artificial Sequence: Synthetic oligonucleotide
  source 1..18: mol_type = other DNA
tcagttggtgctggtatc

SEQ ID NO: 292 - protein, 6 aa, synthetic construct
  REGION 1..6: note = Description of Artificial Sequence: Synthetic peptide
SVGAGI

SEQ ID NO: 293 - DNA, 42 bp, synthetic construct
  misc_feature 1..42: note = Description of Artificial Sequence: Synthetic oligonucleotide
  source 1..42: mol_type = other DNA
gccagcagccatctgggggcgggagggccgcacgagcagtac

SEQ ID NO: 294 - protein, 14 aa, synthetic construct
  REGION 1..14: note = Description of Artificial Sequence: Synthetic peptide
ASSHLGAGGPHEQY

SEQ ID NO: 295 - DNA, 1397 bp, synthetic construct
  misc_feature 1..1397: note = Description of Artificial Sequence: Synthetic polynucleotide
  source 1..1397: mol_type = other DNA
ggtgactgggggaatggaggaggctggagcatgaatggggatggcactggggaccctgacttgcaggaaatgcaatgagctcaccactttgtgccctatgttaggggcggtgttggtgcatcctagagtaaatgtccagcagacaaaggagcaagaagcggctgtgggatgacaagataaactcagagatacagcatgagacctccgggtccagacagctctggagcccaaggcgatgagccatgcattgatgttgttaaaaaggagctgataaatatttaaagcagcacccaactgtgttctaatagaaatgctgtgatcctgaggtcctggggattgagagaggaagtgatgtcactgtgggaactgccctgtggagacaaggacatccctcatcctctgctgctgctcacagtgacactgatctggtaaagccctcatcctgtcctgaccctgccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagccatactgacggctcctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 296 - DNA, 15 bp, synthetic construct
  misc_feature 1..15: note = Description of Artificial Sequence: Synthetic oligonucleotide
  source 1..15: mol_type = other DNA
tcgggtcatgtatcc

SEQ ID NO: 297 - protein, 5 aa, synthetic construct
  REGION 1..5: note = Description of Artificial Sequence: Synthetic peptide
SGHVS

SEQ ID NO: 298 - DNA, 18 bp, synthetic construct
  misc_feature 1..18: note = Description of Artificial Sequence: Synthetic oligonucleotide
  source 1..18: mol_type = other DNA
ttcaattatgaagcccaa

SEQ ID NO: 299 - protein, 6 aa, synthetic construct
  REGION 1..6: note = Description of Artificial Sequence: Synthetic peptide
FNYEAQ

SEQ ID NO: 300 - DNA, 36 bp, synthetic construct
  misc_feature 1..36: note = Description of Artificial Sequence: Synthetic oligonucleotide
  source 1..36: mol_type = other DNA
gccagcagccatactgacggctcctacgagcagtac

SEQ ID NO: 301 - protein, 12 aa, synthetic construct
  REGION 1..12: note = Description of Artificial Sequence: Synthetic peptide
ASSHTDGSYEQY

SEQ ID NO: 302 - DNA, 1168 bp, synthetic construct
  misc_feature 1..1168: note = Description of Artificial Sequence: Synthetic polynucleotide
  source 1..1168: mol_type = other DNA
tggggacagatgcaatgcagagttatgggaggtgcgaatgactctgctctctgtcctgtctcctcatctgcaaaattaggaagcctgtcttgattatctccaggaacctcccacctcttcattccagcctctgacaaactctgcacattaggccaggagaagcccccgagccaagtctcttttctcattctcttccaacaagtgcttggagctccaagaaggccccctttgcactatgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcataggggcatttgctggtcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 303 - DNA, 15 bp, synthetic construct
  misc_feature 1..15: note = Description of Artificial Sequence: Synthetic oligonucleotide
  source 1..15: mol_type = other DNA
ttgaaccacgatgcc

SEQ ID NO: 304 - protein, 5 aa, synthetic construct
  REGION 1..5: note = Description of Artificial Sequence: Synthetic peptide
LNHDA

SEQ ID NO: 305 - DNA, 18 bp, synthetic construct
  misc_feature 1..18: note = Description of Artificial Sequence: Synthetic oligonucleotide
  source 1..18: mol_type = other DNA
tcacagatagtaaatgac

SEQ ID NO: 306 - protein, 6 aa, synthetic construct
  REGION 1..6: note = Description of Artificial Sequence: Synthetic peptide
SQIVND

SEQ ID NO: 307 - DNA, 39 bp, synthetic construct
  misc_feature 1..39: note = Description of Artificial Sequence: Synthetic oligonucleotide
  source 1..39: mol_type = other DNA
gccagtagcataggggcatttgctggtcagccccagcat

SEQ ID NO: 308 - protein, 13 aa, synthetic construct
  REGION 1..13: note = Description of Artificial Sequence: Synthetic peptide
ASSIGAFAGQPQH

SEQ ID NO: 309 - DNA, 1061 bp, synthetic construct
  misc_feature 1..1061: note = Description of Artificial Sequence: Synthetic polynucleotide
  source 1..1061: mol_type = other DNA
tgggggtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagcgcctacccccgggccggacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 310 - DNA, 15 bp, synthetic construct
  misc_feature 1..15: note = Description of Artificial Sequence: Synthetic oligonucleotide
  source 1..15: mol_type = other DNA
tctgaacacaaccgc

SEQ ID NO: 311 - protein, 5 aa, synthetic construct
  REGION 1..5: note = Description of Artificial Sequence: Synthetic peptide
SEHNR

SEQ ID NO: 312 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 313 - protein, 6 aa, synthetic construct
  REGION 1..6: note = Description of Artificial Sequence: Synthetic peptide
FQNEAQ

SEQ ID NO: 314 - DNA, 48 bp, synthetic construct
  misc_feature 1..48: note = Description of Artificial Sequence: Synthetic oligonucleotide
  source 1..48: mol_type = other DNA
gccagcagcttagcgcctacccccgggccggacaccggggagctgttt

SEQ ID NO: 315 - protein, 16 aa, synthetic construct
  REGION 1..16: note = Description of Artificial Sequence: Synthetic peptide
ASSLAPTPGPDTGELF

SEQ ID NO: 316 - DNA, 1081 bp, synthetic construct
  misc_feature 1..1081: note = Description of Artificial Sequence: Synthetic polynucleotide
  source 1..1081: mol_type = other DNA
ggcgtttgtacacttcagccctctgagctgaagtgggagtggtttctctcctgaaaatgtttctgaggcccaaatagctgaagaggtggagacgttacagaaaccacctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttagggaatatctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 317 - DNA, 15 bp, synthetic construct
  misc_feature 1..15: note = Description of Artificial Sequence: Synthetic oligonucleotide
  source 1..15: mol_type = other DNA
atgaaccatgagtat

SEQ ID NO: 318 - protein, 5 aa, synthetic construct
  REGION 1..5: note = Description of Artificial Sequence: Synthetic peptide
MNHEY

SEQ ID NO: 319 - DNA, 18 bp, synthetic construct
  misc_feature 1..18: note = Description of Artificial Sequence: Synthetic oligonucleotide
  source 1..18: mol_type = other DNA
tcaatgaatgttgaggtg

SEQ ID NO: 320 - protein, 6 aa, synthetic construct
  REGION 1..6: note = Description of Artificial Sequence: Synthetic peptide
SMNVEV

SEQ ID NO: 321 - DNA, 33 bp, synthetic construct
  misc_feature 1..33: note = Description of Artificial Sequence: Synthetic oligonucleotide
  source 1..33: mol_type = other DNA
gccagcagtttagggaatatctacgagcagtac

SEQ ID NO: 322 - protein, 11 aa, synthetic construct
  REGION 1..11: note = Description of Artificial Sequence: Synthetic peptide
ASSLGNIYEQY

SEQ ID NO: 323 - DNA, 1055 bp, synthetic construct
  misc_feature 1..1055: note = Description of Artificial Sequence: Synthetic polynucleotide
  source 1..1055: mol_type = other DNA
gagagaggaagtgatgtcactgtgggtactgttctgtgtcaggacaaggacgtccctcctcctctgctcctgctcacagtgaccctgatctggtaaagctcccatcctgccctgactctgtcatgggcaccaggctcctctgctgggcagccctgtgcctcctgggggcagatcacacaggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttagggagcggtgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 324 - DNA, 15 bp, synthetic construct
  misc_feature 1..15: note = Description of Artificial Sequence: Synthetic oligonucleotide
  source 1..15: mol_type = other DNA
tcaggtcatactgcc

SEQ ID NO: 325 - protein, 5 aa, synthetic construct
  REGION 1..5: note = Description of Artificial Sequence: Synthetic peptide
SGHTA

SEQ ID NO: 326 - DNA, 18 bp, synthetic construct
  misc_feature 1..18: note = Description of Artificial Sequence: Synthetic oligonucleotide
  source 1..18: mol_type = other DNA
ttccaaggcacgggtgcg

SEQ ID NO: 327 - protein, 6 aa, synthetic construct
  REGION 1..6: note = Description of Artificial Sequence: Synthetic peptide
FQGTGA

SEQ ID NO: 328 - DNA, 30 bp, synthetic construct
  misc_feature 1..30: note = Description of Artificial Sequence: Synthetic oligonucleotide
  source 1..30: mol_type = other DNA
gccagcagcttagggagcggtgagcagttc

SEQ ID NO: 329 - protein, 10 aa, synthetic construct
  REGION 1..10: note = Description of Artificial Sequence: Synthetic peptide
ASSLGSGEQF

SEQ ID NO: 330 - DNA, 1233 bp, synthetic construct
  misc_feature 1..1233: note = Description of Artificial Sequence: Synthetic polynucleotide
  source 1..1233: mol_type = other DNA
tgggggtcttgggtgcttgcttccccttccttctgccttggataacagcaggaggccctcatcagttatggacccttgatcttggacttcccagtctccagatctacaacccagctctgctttggatctgatcagatggactaaatcttggggactctgcaccactggccactgaggaaagggaagagaatgttgcctgggacaggaaaatatagaaaatgaaggcccagaactcactcggctcttccccaggaagaccaagccctgaatcaggtgcagtgccgcctggcccactgtgccatgggacccaggctcctcttctgggcactgctttgtctcctcggaacaggcccagtggaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagcttacagacatcctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 331 - DNA, 15 bp, synthetic construct
  misc_feature 1..15: note = Description of Artificial Sequence: Synthetic oligonucleotide
  source 1..15: mol_type = other DNA
tctgggcacaccagt

SEQ ID NO: 332 - protein, 5 aa, synthetic construct
  REGION 1..5: note = Description of Artificial Sequence: Synthetic peptide
SGHTS

SEQ ID NO: 333 - DNA, 18 bp, synthetic construct
  misc_feature 1..18: note = Description of Artificial Sequence: Synthetic oligonucleotide
  source 1..18: mol_type = other DNA
tatgacgagggtgaagag

SEQ ID NO: 334 - protein, 6 aa, synthetic construct
  REGION 1..6: note = Description of Artificial Sequence: Synthetic peptide
YDEGEE

SEQ ID NO: 335 - DNA, 33 bp, synthetic construct
  misc_feature 1..33: note = Description of Artificial Sequence: Synthetic oligonucleotide
  source 1..33: mol_type = other DNA
gccagcagcttacagacatcctacgagcagtac

SEQ ID NO: 336 - protein, 11 aa, synthetic construct
  REGION 1..11: note = Description of Artificial Sequence: Synthetic peptide
ASSLQTSYEQY

SEQ ID NO: 337 - DNA, 1120 bp, synthetic construct
  misc_feature 1..1120: note = Description of Artificial Sequence: Synthetic polynucleotide
  source 1..1120: mol_type = other DNA
gggctcagcctgagttggctgtgttgcgtttgtacacttcagccctctgagctgaagtgggagtggtttctctcctgaaaatgtttctgaggcccaaatagctgaagaggtggagacgttacagaaaccacctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcgtgggggaccggcaaaagagcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 338 - DNA, 15 bp, synthetic construct
  misc_feature 1..15: note = Description of Artificial Sequence: Synthetic oligonucleotide
  source 1..15: mol_type = other DNA
atgaaccatgagtat

SEQ ID NO: 339 - protein, 5 aa, synthetic construct
  REGION 1..5: note = Description of Artificial Sequence: Synthetic peptide
MNHEY

SEQ ID NO: 340 - DNA, 18 bp, synthetic construct
  misc_feature 1..18: note = Description of Artificial Sequence: Synthetic oligonucleotide
  source 1..18: mol_type = other DNA
tcaatgaatgttgaggtg

SEQ ID NO: 341 - protein, 6 aa, synthetic construct
  REGION 1..6: note = Description of Artificial Sequence: Synthetic peptide
SMNVEV

SEQ ID NO: 342 - DNA, 48 bp, synthetic construct
  misc_feature 1..48: note = Description of Artificial Sequence: Synthetic oligonucleotide
  source 1..48: mol_type = other DNA
gccagcagtttatcgtgggggaccggcaaaagagcagatacgcagtat

SEQ ID NO: 343 - protein, 16 aa, synthetic construct
  REGION 1..16: note = Description of Artificial Sequence: Synthetic peptide
ASSLSWGTGKRADTQY

SEQ ID NO: 344 - DNA, 1321 bp, synthetic construct
  misc_feature 1..1321: note = Description of Artificial Sequence: Synthetic polynucleotide
  source 1..1321: mol_type = other DNA
cgaaggcggagggtggaatgcgggcagcagccccctggagggctgagtggggaaaacaaaatggacctcacagaagctgtgtgtgtggaaacccacttctgacttatcacttgtcatgaattctatgcttcatggtgttacaccgtttattgtttctgatgagtgacagtaattattttctttcttgctggtacataataaagtggtgcacatcagagttgctgccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagttggcggcgggagcaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 345 - DNA, 15 bp, synthetic construct
  misc_feature 1..15: note = Description of Artificial Sequence: Synthetic oligonucleotide
  source 1..15: mol_type = other DNA
tctgaacacaaccgc

SEQ ID NO: 346 - protein, 5 aa, synthetic construct
  REGION 1..5: note = Description of Artificial Sequence: Synthetic peptide
SEHNR

SEQ ID NO: 347 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 348 - protein, 6 aa, synthetic construct
  REGION 1..6: note = Description of Artificial Sequence: Synthetic peptide
FQNEAQ

SEQ ID NO: 349 - DNA, 39 bp, synthetic construct
  misc_feature 1..39: note = Description of Artificial Sequence: Synthetic oligonucleotide
  source 1..39: mol_type = other DNA
gccagcagcttagttggcggcgggagcaatgagcagttc

SEQ ID NO: 350 - protein, 13 aa, synthetic construct
  REGION 1..13: note = Description of Artificial Sequence: Synthetic peptide
ASSLVGGGSNEQF

SEQ ID NO: 351 - DNA, 1487 bp, synthetic construct
  misc_feature 1..1487: note = Description of Artificial Sequence: Synthetic polynucleotide
  source 1..1487: mol_type = other DNA
gacctcccactcctacccagaccgtggatgggcaggaaatgcaggaacagagccagaaacaggagatctccaaggaaggttgacagtcagcactgggatcgtctgtgtaaagtgctgctgaagcagccaggtggcatgtccagccgacaatgcgaaaggaaaaagtgagaagacttcccgaaggcggagggtggaatgcgggcagcagccccctggagggctgagtggggaaaacaaaatggacctcacagaagctgtgtgtgtggaaacccacttctgacttatcacttgtcatgaattctatgcttcatggtgttacaccgtttattgtttctgatgagtgacagtaattattttctttcttgctggtacataataaagtggtgcacatcagagttgctgccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagtcggaggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 352 - DNA, 15 bp, synthetic construct
  misc_feature 1..15: note = Description of Artificial Sequence: Synthetic oligonucleotide
  source 1..15: mol_type = other DNA
tctgaacacaaccgc

SEQ ID NO: 353 - protein, 5 aa, synthetic construct
  REGION 1..5: note = Description of Artificial Sequence: Synthetic peptide
SEHNR

SEQ ID NO: 354 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 355 - protein, 6 aa, synthetic construct
  REGION 1..6: note = Description of Artificial Sequence: Synthetic peptide
FQNEAQ

SEQ ID NO: 356 - DNA, 33 bp, synthetic construct
  misc_feature 1..33: note = Description of Artificial Sequence: Synthetic oligonucleotide
  source 1..33: mol_type = other DNA
gccagcagcttagtcggaggcactgaagctttc

SEQ ID NO: 357 - protein, 11 aa, synthetic construct
  REGION 1..11: note = Description of Artificial Sequence: Synthetic peptide
ASSLVGGTEAF

SEQ ID NO: 358 - DNA, 1094 bp, synthetic construct
  misc_feature 1..1094: note = Description of Artificial Sequence: Synthetic polynucleotide
  source 1..1094: mol_type = other DNA
gggccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccgatcggaatctcgggcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 359 - DNA, 15 bp, synthetic construct
  misc_feature 1..15: note = Description of Artificial Sequence: Synthetic oligonucleotide
  source 1..15: mol_type = other DNA
tctgaacacaaccgc

SEQ ID NO: 360 - protein, 5 aa, synthetic construct
  REGION 1..5: note = Description of Artificial Sequence: Synthetic peptide
SEHNR

SEQ ID NO: 361 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 362 - protein, 6 aa, synthetic construct
  REGION 1..6: note = Description of Artificial Sequence: Synthetic peptide
FQNEAQ

SEQ ID NO: 363 - DNA, 33 bp, synthetic construct
  misc_feature 1..33: note = Description of Artificial Sequence: Synthetic oligonucleotide
  source 1..33: mol_type = other DNA
gccagcagccccgatcggaatctcgggcagtac

SEQ ID NO: 364 - protein, 11 aa, synthetic construct
  REGION 1..11: note = Description of Artificial Sequence: Synthetic peptide
ASSPDRNLGQY

SEQ ID NO: 365 - DNA, 1067 bp, synthetic construct
  misc_feature 1..1067: note = Description of Artificial Sequence: Synthetic polynucleotide
  source 1..1067: mol_type = other DNA
gggacattggctaatatgctgatgtcactggaggccacatcttacagggccaagagacagatttgctttcctttttctcatgcttgtaagctccttcatctggaaatgtgatttacctgggtcctgccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgaatccggtatgcccaacaatcgattcttagctgaaaggactggagggacgtattctactctgaaggtgcagcctgcagaactggaggattctggagtttatttctgtgccagcagccaagtccaggcttttaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 366 - DNA, 15 bp, synthetic construct
  misc_feature 1..15: note = Description of Artificial Sequence: Synthetic oligonucleotide
  source 1..15: mol_type = other DNA
tctggacatgataat

SEQ ID NO: 367 - protein, 5 aa, synthetic construct
  REGION 1..5: note = Description of Artificial Sequence: Synthetic peptide
SGHDN

SEQ ID NO: 368 - DNA, 18 bp, synthetic construct
  misc_feature 1..18: note = Description of Artificial Sequence: Synthetic oligonucleotide
  source 1..18: mol_type = other DNA
tttgtgaaagagtctaaa

SEQ ID NO: 369 - protein, 6 aa, synthetic construct
  REGION 1..6: note = Description of Artificial Sequence: Synthetic peptide
FVKESK

SEQ ID NO: 370 - DNA, 36 bp, synthetic construct
  misc_feature 1..36: note = Description of Artificial Sequence: Synthetic oligonucleotide
  source 1..36: mol_type = other DNA
gccagcagccaagtccaggcttttaatgagcagttc

SEQ ID NO: 371 - protein, 12 aa, synthetic construct
  REGION 1..12: note = Description of Artificial Sequence: Synthetic peptide
ASSQVQAFNEQF

SEQ ID NO: 372 - DNA, 1222 bp, synthetic construct
  misc_feature 1..1222: note = Description of Artificial Sequence: Synthetic polynucleotide
  source 1..1222: mol_type = other DNA
tctcttttatgttgccatccaagtaagacatgacttgcccctccttgccttctgccatgattgtgaggcctccccagccatgtggaactaggaaaagtgaatcaaaaccaagggacatgctgagacaactggagaaatttgaatgtgaagcatttgtggaggcaatgatgtcactgtgggaactgccatgagaggacagggacgtccctcctcctctgcttttgctcacagtgaccctgattgggcaaagctcccatccttccctgaccctgccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagtcgaggggagccaggctctggggccaacgtcctgactttcggggccggcagcaggctgaccgtgctggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 373 - DNA, 15 bp, synthetic construct
  misc_feature 1..15: note = Description of Artificial Sequence: Synthetic oligonucleotide
  source 1..15: mol_type = other DNA
tctggccatgctacc

SEQ ID NO: 374 - protein, 5 aa, synthetic construct
  REGION 1..5: note = Description of Artificial Sequence: Synthetic peptide
SGHAT

SEQ ID NO: 375 - DNA, 18 bp, synthetic construct
  misc_feature 1..18: note = Description of Artificial Sequence: Synthetic oligonucleotide
  source 1..18: mol_type = other DNA
tttcagaataacggtgta

SEQ ID NO: 376 - protein, 6 aa, synthetic construct
  REGION 1..6: note = Description of Artificial Sequence: Synthetic peptide
FQNNGV

SEQ ID NO: 377 - DNA, 45 bp, synthetic construct
  misc_feature 1..45: note = Description of Artificial Sequence: Synthetic oligonucleotide
  source 1..45: mol_type = other DNA
gccagcagtcgaggggagccaggctctggggccaacgtcctgact

SEQ ID NO: 378 - protein, 15 aa, synthetic construct
  REGION 1..15: note = Description of Artificial Sequence: Synthetic peptide
ASSRGEPGSGANVLT

SEQ ID NO: 379 - DNA, 1203 bp, synthetic construct
  misc_feature 1..1203: note = Description of Artificial Sequence: Synthetic polynucleotide
  source 1..1203: mol_type = other DNA
ggggcagtgtaggcagaggaggaactgtatcaccacagaaacttctgccttcacacatccctccagctaggcaggacaggtagagagtccagtgtcctggagcactagacctaaggaaggctgcatggggaggacaaaggacagtgacatcacaggatacccctcccatcaggaaaatcaaggcccagaactcactcggctcttccccaggagaaccaagccctgaatcagatgcagtgcttcctgtccctctgtgccatgggccccgggctcctctgctgggcactgctttgtctcctgggagcaggcttagtggacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctccgggacagacccctctggggccaacgtcctgactttcggggccggcagcaggctgaccgtgctggaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 380 - DNA, 15 bp, synthetic construct
  misc_feature 1..15: note = Description of Artificial Sequence: Synthetic oligonucleotide
  source 1..15: mol_type = other DNA
tctgggcatgacact

SEQ ID NO: 381 - protein, 5 aa, synthetic construct
  REGION 1..5: note = Description of Artificial Sequence: Synthetic peptide
SGHDT

SEQ ID NO: 382 - DNA, 18 bp, synthetic construct
  misc_feature 1..18: note = Description of Artificial Sequence: Synthetic oligonucleotide
  source 1..18: mol_type = other DNA
tattatgaggaggaagag

SEQ ID NO: 383 - protein, 6 aa, synthetic construct
  REGION 1..6: note = Description of Artificial Sequence: Synthetic peptide
YYEEEE

SEQ ID NO: 384 - DNA, 45 bp, synthetic construct
  misc_feature 1..45: note = Description of Artificial Sequence: Synthetic oligonucleotide
  source 1..45: mol_type = other DNA
gccagcagctccgggacagacccctctggggccaacgtcctgact

SEQ ID NO: 385 - protein, 15 aa, synthetic construct
  REGION 1..15: note = Description of Artificial Sequence: Synthetic peptide
ASSSGTDPSGANVLT

SEQ ID NO: 386 - DNA, 1087 bp, synthetic construct
  misc_feature 1..1087: note = Description of Artificial Sequence: Synthetic polynucleotide
  source 1..1087: mol_type = other DNA
ggggtttgtacacttcagccctctgagctgaagtgggagtggtttctctcctgaaaatgtttctgaggcccaaatagctgaagaggtggagacgttacagaaaccacctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtaccggacagaatataggcggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 387 - DNA, 15 bp, synthetic construct
  misc_feature 1..15: note = Description of Artificial Sequence: Synthetic oligonucleotide
  source 1..15: mol_type = other DNA
atgaaccatgagtat

SEQ ID NO: 388 - protein, 5 aa, synthetic construct
  REGION 1..5: note = Description of Artificial Sequence: Synthetic peptide
MNHEY

SEQ ID NO: 389 - DNA, 18 bp, synthetic construct
  misc_feature 1..18: note = Description of Artificial Sequence: Synthetic oligonucleotide
  source 1..18: mol_type = other DNA
tcaatgaatgttgaggtg

SEQ ID NO: 390 - protein, 6 aa, synthetic construct
  REGION 1..6: note = Description of Artificial Sequence: Synthetic peptide
SMNVEV

SEQ ID NO: 391 - DNA, 39 bp, synthetic construct
  misc_feature 1..39: note = Description of Artificial Sequence: Synthetic oligonucleotide
  source 1..39: mol_type = other DNA
gccagcagtaccggacagaatataggcggggagctgttt

SEQ ID NO: 392 - protein, 13 aa, synthetic construct
  REGION 1..13: note = Description of Artificial Sequence: Synthetic peptide
ASSTGQNIGGELF

SEQ ID NO: 393 - DNA, 1232 bp, synthetic construct
  misc_feature 1..1232: note = Description of Artificial Sequence: Synthetic polynucleotide
  source 1..1232: mol_type = other DNA
tgggagaaacaggagatctccaaggaaggttgacagtcagcactgggatcgtctgtgtaaagtgctgctgaagcagccaggtggcatgtccagccgacaatgcgaaaggaaaaatggtgcacatcagagttgctgccatcttagacttaactcatcagtatcaggtgatcctgaggctcagtgatgtcactgtgggaactgctctgtggcgacaaggacgtccctcatcctctgctcctgctcacagtgaccctgatctggtaaagctcccatcctgccctgaccctgccatgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcacacatgaaaagacagggtggaaatcacccctccactttgggaatgggaccaggctcactgtgacagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 394 - DNA, 15 bp, synthetic construct
  misc_feature 1..15: note = Description of Artificial Sequence: Synthetic oligonucleotide
  source 1..15: mol_type = other DNA
tctgaacacaaccgc

SEQ ID NO: 395 - protein, 5 aa, synthetic construct
  REGION 1..5: note = Description of Artificial Sequence: Synthetic peptide
SEHNR

SEQ ID NO: 396 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 397 - protein, 6 aa, synthetic construct
  REGION 1..6: note = Description of Artificial Sequence: Synthetic peptide
FQNEAQ

SEQ ID NO: 398 - DNA, 45 bp, synthetic construct
  misc_feature 1..45: note = Description of Artificial Sequence: Synthetic oligonucleotide
  source 1..45: mol_type = other DNA
gccagcagcacacatgaaaagacagggtggaaatcacccctccac

SEQ ID NO: 399 - protein, 15 aa, synthetic construct
  REGION 1..15: note = Description of Artificial Sequence: Synthetic peptide
ASSTHEKTGWKSPLH

SEQ ID NO: 400 - DNA, 1051 bp, synthetic construct
  misc_feature 1..1051: note = Description of Artificial Sequence: Synthetic polynucleotide
  source 1..1051: mol_type = other DNA
ggggccagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtacgcactctgacaggaacttgaacactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 401 - DNA, 15 bp, synthetic construct
  misc_feature 1..15: note = Description of Artificial Sequence: Synthetic oligonucleotide
  source 1..15: mol_type = other DNA
tctaatcacttatac

SEQ ID NO: 402 - protein, 5 aa, synthetic construct
  REGION 1..5: note = Description of Artificial Sequence: Synthetic peptide
SNHLY

SEQ ID NO: 403 - DNA, 18 bp, synthetic construct
  misc_feature 1..18: note = Description of Artificial Sequence: Synthetic oligonucleotide
  source 1..18: mol_type = other DNA
ttttataataatgaaatc

SEQ ID NO: 404 - protein, 6 aa, synthetic construct
  REGION 1..6: note = Description of Artificial Sequence: Synthetic peptide
FYNNEI

SEQ ID NO: 405 - DNA, 45 bp, synthetic construct
  misc_feature 1..45: note = Description of Artificial Sequence: Synthetic oligonucleotide
  source 1..45: mol_type = other DNA
gccagcagtacgcactctgacaggaacttgaacactgaagctttc

SEQ ID NO: 406 - protein, 15 aa, synthetic construct
  REGION 1..15: note = Description of Artificial Sequence: Synthetic peptide
ASSTHSDRNLNTEAF

SEQ ID NO: 407 - DNA, 1040 bp, synthetic construct
  misc_feature 1..1040: note = Description of Artificial Sequence: Synthetic polynucleotide
  source 1..1040: mol_type = other DNA
tgggagaccttgcctgtggggccatgggagctcaaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagcgtacaggctacgggccatggctacaccttcggttcggggaccaggttaaccgttgtagaggacctgaacaaggtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 408 - DNA, 15 bp, synthetic construct
  misc_feature 1..15: note = Description of Artificial Sequence: Synthetic oligonucleotide
  source 1..15: mol_type = other DNA
tctaatcacttatac

SEQ ID NO: 409 - protein, 5 aa, synthetic construct
  REGION 1..5: note = Description of Artificial Sequence: Synthetic peptide
SNHLY

SEQ ID NO: 410 - DNA, 18 bp, synthetic construct
  misc_feature 1..18: note = Description of Artificial Sequence: Synthetic oligonucleotide
  source 1..18: mol_type = other DNA
ttttataataatgaaatc

SEQ ID NO: 411 - protein, 6 aa, synthetic construct
  REGION 1..6: note = Description of Artificial Sequence: Synthetic peptide
FYNNEI

SEQ ID NO: 412 - DNA, 36 bp, synthetic construct
  misc_feature 1..36: note = Description of Artificial Sequence: Synthetic oligonucleotide
  source 1..36: mol_type = other DNA
gccagcagcgtacaggctacgggccatggctacacc

SEQ ID NO: 413 - protein, 12 aa, synthetic construct
  REGION 1..12: note = Description of Artificial Sequence: Synthetic peptide
ASSVQATGHGYT

SEQ ID NO: 414 - DNA, 1045 bp, synthetic construct
  misc_feature 1..1045: note = Description of Artificial Sequence: Synthetic polynucleotide
  source 1..1045: mol_type = other DNA
tggggtcactgtgggaactgccctgtggagacaaggacggcccttatcctctgcttctgttcacagtgacactgatctggtaaagcccccatcctggcctgaccctgccatgggcaccaggctcctctgctgggtggtcctgggtttcctagggacagatcacacaggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcaccccctctggctataactcttgggagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 415 - DNA, 15 bp, synthetic construct
  misc_feature 1..15: note = Description of Artificial Sequence: Synthetic oligonucleotide
  source 1..15: mol_type = other DNA
tcgggtcatgtatcc

SEQ ID NO: 416 - protein, 5 aa, synthetic construct
  REGION 1..5: note = Description of Artificial Sequence: Synthetic peptide
SGHVS

SEQ ID NO: 417 - DNA, 18 bp, synthetic construct
  misc_feature 1..18: note = Description of Artificial Sequence: Synthetic oligonucleotide
  source 1..18: mol_type = other DNA
ttccagaatgaagctcaa

SEQ ID NO: 418 - protein, 6 aa, synthetic construct
  REGION 1..6: note = Description of Artificial Sequence: Synthetic peptide
FQNEAQ

SEQ ID NO: 419 - DNA, 39 bp, synthetic construct
  misc_feature 1..39: note = Description of Artificial Sequence: Synthetic oligonucleotide
  source 1..39: mol_type = other DNA
gccagcaccccctctggctataactcttgggagcagttc

SEQ ID NO: 420 - protein, 13 aa, synthetic construct
  REGION 1..13: note = Description of Artificial Sequence: Synthetic peptide
ASTPSGYNSWEQF

SEQ ID NO: 421 - DNA, 1082 bp, synthetic construct
  misc_feature 1..1082: note = Description of Artificial Sequence: Synthetic polynucleotide
  source 1..1082: mol_type = other DNA
gtgttgcgtttgtacacttcagccctctgagctgaagtgggagtggtttctctcctgaaaatgtttctgaggcccaaatagctgaagaggtggagacgttacagaaaccacctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccaccgcccccaggggtagcaatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 422 - DNA, 15 bp, synthetic construct
  misc_feature 1..15: note = Description of Artificial Sequence: Synthetic oligonucleotide
  source 1..15: mol_type = other DNA
atgaaccatgagtat

SEQ ID NO: 423 - protein, 5 aa, synthetic construct
  REGION 1..5: note = Description of Artificial Sequence: Synthetic peptide
MNHEY

SEQ ID NO: 424 - DNA, 18 bp, synthetic construct
  misc_feature 1..18: note = Description of Artificial Sequence: Synthetic oligonucleotide
  source 1..18: mol_type = other DNA
tcaatgaatgttgaggtg

SEQ ID NO: 425 - protein, 6 aa, synthetic construct
  REGION 1..6: note = Description of Artificial Sequence: Synthetic peptide
SMNVEV

SEQ ID NO: 426 - DNA, 36 bp, synthetic construct
  misc_feature 1..36: note = Description of Artificial Sequence: Synthetic oligonucleotide
  source 1..36: mol_type = other DNA
gccaccgcccccaggggtagcaatcagccccagcat

SEQ ID NO: 427 - protein, 12 aa, synthetic construct
  REGION 1..12: note = Description of Artificial Sequence: Synthetic peptide
ATAPRGSNQPQH

SEQ ID NO: 428 - DNA, 1021 bp, synthetic construct
  misc_feature 1..1021: note = Description of Artificial Sequence: Synthetic polynucleotide
  source 1..1021: mol_type = other DNA
gggggggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagggcctggggcgggagctcctacaatgagcagttcttcgggccagggacacggctcaccgtgctagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 429 - DNA, 18 bp, synthetic construct
  misc_feature 1..18: note = Description of Artificial Sequence: Synthetic oligonucleotide
  source 1..18: mol_type = other DNA
gactttcaggccacaact

SEQ ID NO: 430 - protein, 6 aa, synthetic construct
  REGION 1..6: note = Description of Artificial Sequence: Synthetic peptide
DFQATT

SEQ ID NO: 431 - DNA, 21 bp, synthetic construct
  misc_feature 1..21: note = Description of Artificial Sequence: Synthetic oligonucleotide
  source 1..21: mol_type = other DNA
tccaatgagggctccaaggcc

SEQ ID NO: 432 - protein, 7 aa, synthetic construct
  REGION 1..7: note = Description of Artificial Sequence: Synthetic peptide
SNEGSKA

SEQ ID NO: 433 - DNA, 36 bp, synthetic construct
  misc_feature 1..36: note = Description of Artificial Sequence: Synthetic oligonucleotide
  source 1..36: mol_type = other DNA
agggcctggggcgggagctcctacaatgagcagttc

SEQ ID NO: 434 - protein, 12 aa, synthetic construct
  REGION 1..12: note = Description of Artificial Sequence: Synthetic peptide
RAWGGSSYNEQF

SEQ ID NO: 435 - DNA, 1017 bp, synthetic construct
  misc_feature 1..1017: note = Description of Artificial Sequence: Synthetic polynucleotide
  source 1..1017: mol_type = other DNA
tggggagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaattggacagggttttatgaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 436 - DNA, 18 bp, synthetic construct
  misc_feature 1..18: note = Description of Artificial Sequence: Synthetic oligonucleotide
  source 1..18: mol_type = other DNA
gactttcaggccacaact

SEQ ID NO: 437 - protein, 6 aa, synthetic construct
  REGION 1..6: note = Description of Artificial Sequence: Synthetic peptide
DFQATT

SEQ ID NO: 438 - DNA, 21 bp, synthetic construct
  misc_feature 1..21: note = Description of Artificial Sequence: Synthetic oligonucleotide
  source 1..21: mol_type = other DNA
tccaatgagggctccaaggcc

SEQ ID NO: 439 - protein, 7 aa, synthetic construct
  REGION 1..7: note = Description of Artificial Sequence: Synthetic peptide
SNEGSKA

SEQ ID NO: 440 - DNA, 39 bp, synthetic construct
  misc_feature 1..39: note = Description of Artificial Sequence: Synthetic oligonucleotide
  source 1..39: mol_type = other DNA
agtgctagaattggacagggttttatgaatgagcagttc

SEQ ID NO: 441 - protein, 13 aa, synthetic construct
  REGION 1..13: note = Description of Artificial Sequence: Synthetic peptide
SARIGQGFMNEQF

SEQ ID NO: 442 - DNA, 1060 bp, synthetic construct
  misc_feature 1..1060: note = Description of Artificial Sequence: Synthetic polynucleotide
  source 1..1060: mol_type = other DNA
agtcatgggcaaagattaccaccagggggcagactagggcatccttgggattctgtgatcagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcctgggtggcgtggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 443 - DNA, 18 bp, synthetic construct
  misc_feature 1..18: note = Description of Artificial Sequence: Synthetic oligonucleotide
  source 1..18: mol_type = other DNA
gactttcaggccacaact

SEQ ID NO: 444 - protein, 6 aa, synthetic construct
  REGION 1..6: note = Description of Artificial Sequence: Synthetic peptide
DFQATT

SEQ ID NO: 445 - DNA, 21 bp, synthetic construct
  misc_feature 1..21: note = Description of Artificial Sequence: Synthetic oligonucleotide
  source 1..21: mol_type = other DNA
tccaatgagggctccaaggcc

SEQ ID NO: 446 - protein, 7 aa, synthetic construct
  REGION 1..7: note = Description of Artificial Sequence: Synthetic peptide
SNEGSKA

SEQ ID NO: 447 - DNA, 33 bp, synthetic construct
  misc_feature 1..33: note = Description of Artificial Sequence: Synthetic oligonucleotide
  source 1..33: mol_type = other DNA
agtgctcctgggtggcgtggcactgaagctttc

SEQ ID NO: 448 - protein, 11 aa, synthetic construct
  REGION 1..11: note = Description of Artificial Sequence: Synthetic peptide
SAPGWRGTEAF

SEQ ID NO: 449 - DNA, 1038 bp, synthetic construct
  misc_feature 1..1038: note = Description of Artificial Sequence: Synthetic polynucleotide
  source 1..1038: mol_type = other DNA
gggactggcgcagcacctctcagcggcagtggaaaccacagcctagtcctctcaccactgcagaccagaatcctgccctgggccttgcctggtctgcctcactctgccatgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagatggacttgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 450 - DNA, 15 bp, synthetic construct
  misc_feature 1..15: note = Description of Artificial Sequence: Synthetic oligonucleotide
  source 1..15: mol_type = other DNA
ctgggccatgatact

SEQ ID NO: 451 - protein, 5 aa, synthetic construct
  REGION 1..5: note = Description of Artificial Sequence: Synthetic peptide
LGHDT

SEQ ID NO: 452 - DNA, 18 bp, synthetic construct
  misc_feature 1..18: note = Description of Artificial Sequence: Synthetic oligonucleotide
  source 1..18: mol_type = other DNA
tacaataataaggagctc

SEQ ID NO: 453 - protein, 6 aa, synthetic construct
  REGION 1..6: note = Description of Artificial Sequence: Synthetic peptide
YNNKEL

SEQ ID NO: 454 - DNA, 30 bp, synthetic construct
  misc_feature 1..30: note = Description of Artificial Sequence: Synthetic oligonucleotide
  source 1..30: mol_type = other DNA
gccagcagccaagatggacttgagcagtac

SEQ ID NO: 455 - protein, 10 aa, synthetic construct
  REGION 1..10: note = Description of Artificial Sequence: Synthetic peptide
ASSQDGLEQY

SEQ ID NO: 456 - DNA, 1068 bp, synthetic construct
  misc_feature 1..1068: note = Description of Artificial Sequence: Synthetic polynucleotide
  source 1..1068: mol_type = other DNA
gacctcactggcgcagcacctctcagcggcagtggaaaccacagcctagtcctctcaccactgcagaccagaatcctgccctgggccttgcctggtctgcctcactctgccatgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagaaggatctcaggaccacactagcgggagggccaccgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 457 - DNA, 15 bp, synthetic construct
  misc_feature 1..15: note = Description of Artificial Sequence: Synthetic oligonucleotide
  source 1..15: mol_type = other DNA
ctgggccatgatact

SEQ ID NO: 458 - protein, 5 aa, synthetic construct
  REGION 1..5: note = Description of Artificial Sequence: Synthetic peptide
LGHDT

SEQ ID NO: 459 - DNA, 18 bp, synthetic construct
  misc_feature 1..18: note = Description of Artificial Sequence: Synthetic oligonucleotide
  source 1..18: mol_type = other DNA
tacaataataaggagctc

SEQ ID NO: 460 - protein, 6 aa, synthetic construct
  REGION 1..6: note = Description of Artificial Sequence: Synthetic peptide
YNNKEL

SEQ ID NO: 461 - DNA, 57 bp, synthetic construct
  misc_feature 1..57: note = Description of Artificial Sequence: Synthetic oligonucleotide
  source 1..57: mol_type = other DNA
gccagcagccaagaaggatctcaggaccacactagcgggagggccaccgagcagtac

SEQ ID NO: 462 - protein, 19 aa, synthetic construct
  REGION 1..19: note = Description of Artificial Sequence: Synthetic peptide
ASSQEGSQDHTSGRATEQY

SEQ ID NO: 463 - DNA, 1057 bp, synthetic construct
  misc_feature 1..1057: note = Description of Artificial Sequence: Synthetic polynucleotide
  source 1..1057: mol_type = other DNA
ggggctctccctccctggggcccaggcagggagaatgtctcagaatgacttccttgagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagcctgacagaaggaagaagcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 464 - DNA, 15 bp, synthetic construct
  misc_feature 1..15: note = Description of Artificial Sequence: Synthetic oligonucleotide
  source 1..15: mol_type = other DNA
atgaaccatgaatac

SEQ ID NO: 465 - protein, 5 aa, synthetic construct
  REGION 1..5: note = Description of Artificial Sequence: Synthetic peptide
MNHEY

SEQ ID NO: 466 - DNA, 18 bp, synthetic construct
  misc_feature 1..18: note = Description of Artificial Sequence: Synthetic oligonucleotide
  source 1..18: mol_type = other DNA
tcagttggtgctggtatc

SEQ ID NO: 467 - protein, 6 aa, synthetic construct
  REGION 1..6: note = Description of Artificial Sequence: Synthetic peptide
SVGAGI

SEQ ID NO: 468 - DNA, 36 bp, synthetic construct
  misc_feature 1..36: note = Description of Artificial Sequence: Synthetic oligonucleotide
  source 1..36: mol_type = other DNA
gccagcagcctgacagaaggaagaagcgagcagtac

SEQ ID NO: 469 - protein, 12 aa, synthetic construct
  REGION 1..12: note = Description of Artificial Sequence: Synthetic peptide
ASSLTEGRSEQY

SEQ ID NO: 470 - DNA, 1004 bp, synthetic construct
  misc_feature 1..1004: note = Description of Artificial Sequence: Synthetic polynucleotide
  source 1..1004: mol_type = other DNA
ggggagtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctgggggtttctctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 471 - DNA, 18 bp, synthetic construct
  misc_feature 1..18: note = Description of Artificial Sequence: Synthetic oligonucleotide
  source 1..18: mol_type = other DNA
gactttcaggccacaact

SEQ ID NO: 472 - protein, 6 aa, synthetic construct
  REGION 1..6: note = Description of Artificial Sequence: Synthetic peptide
DFQATT

SEQ ID NO: 473 - DNA, 21 bp, synthetic construct
  misc_feature 1..21: note = Description of Artificial Sequence: Synthetic oligonucleotide
  source 1..21: mol_type = other DNA
tccaatgagggctccaaggcc

SEQ ID NO: 474 - protein, 7 aa, synthetic construct
  REGION 1..7: note = Description of Artificial Sequence: Synthetic peptide
SNEGSKA

SEQ ID NO: 475 - DNA, 33 bp, synthetic construct
  misc_feature 1..33: note = Description of Artificial Sequence: Synthetic oligonucleotide
  source 1..33: mol_type = other DNA
agtgctgggggtttctctggaaacaccatatat

SEQ ID NO: 476 - protein, 11 aa, synthetic construct
  REGION 1..11: note = Description of Artificial Sequence: Synthetic peptide
SAGGFSGNTIY

SEQ ID NO: 477 - protein, 270 aa, synthetic construct
  REGION 1..270: note = Description of Artificial Sequence: Synthetic polypeptide
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAARSYNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 478 - DNA, 334 bp, synthetic construct
  misc_feature 1..334: note = Description of Artificial Sequence: Synthetic polynucleotide
  source 1..334: mol_type = other DNA
gagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgcccgatcttataacaccgacaagctcatctttgggactgggaccagattacaagtctttccaa

SEQ ID NO: 479 - protein, 111 aa, synthetic construct
  REGION 1..111: note = Description of Artificial Sequence: Synthetic polypeptide
ELKVEQNPLFLSMQEGKNYTIYCNYSTTSDRLYWYRQDPGKSLESLFVLLSNGAVKQEGRLMASLDTKARLSTLHITAAVHDLSATYFCAARSYNTDKLIFGTGTRLQVFP

SEQ ID NO: 480 - protein, 275 aa, synthetic construct
  REGION 1..275: note = Description of Artificial Sequence: Synthetic polypeptide
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMGGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 481 - DNA, 346 bp, synthetic construct
  misc_feature 1..346: note = Description of Artificial Sequence: Synthetic polynucleotide
  source 1..346: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacatggggggttctgcaaggcaactgacctttggatctgggacacaattgactgttttacctg

SEQ ID NO: 482 - protein, 115 aa, synthetic construct
  REGION 1..115: note = Description of Artificial Sequence: Synthetic polypeptide
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDMGGSARQLTFGSGTQLTVLP

SEQ ID NO: 483 - protein, 271 aa, synthetic construct
  REGION 1..271: note = Description of Artificial Sequence: Synthetic polypeptide
NMRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGTFSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 484 - DNA, 337 bp, synthetic construct
  misc_feature 1..337: note = Description of Artificial Sequence: Synthetic polynucleotide
  source 1..337: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtggggacgttttctggttctgcaaggcaactgacctttggatctgggacacaattgactgttttacctg

SEQ ID NO: 485 - protein, 112 aa, synthetic construct
  REGION 1..112: note = Description of Artificial Sequence: Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVGTFSGSARQLTFGSGTQLTVLP

SEQ ID NO: 486 - protein, 277 aa, synthetic construct
  REGION 1..277: note = Description of Artificial Sequence: Synthetic polypeptide
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVSTSMYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 487 - DNA, 343 bp, synthetic construct
  misc_feature 1..343: note = Description of Artificial Sequence: Synthetic polynucleotide
  source 1..343: mol_type = other DNA
aaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagcacctctatgtattcaggaggaggtgctgacggactcacctttggcaaagggactcatctaatcatccagccct

SEQ ID NO: 488 - protein, 114 aa, synthetic construct
  REGION 1..114: note = Description of Artificial Sequence: Synthetic polypeptide
KNEVEQSPQNLTAQEGEFITINCSYSVGISALHWLQQHPGGGIVSLFMLSSGKKKHGRLIATINIQEKHSSLHITASHPRDSAVYICAVSTSMYSGGGADGLTFGKGTHLIIQP

SEQ ID NO: 489 - protein, 272 aa, synthetic construct
  REGION 1..272: note = Description of Artificial Sequence: Synthetic polypeptide
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVKGGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 490 - DNA, 328 bp, synthetic construct
  misc_feature 1..328: note = Description of Artificial Sequence: Synthetic polynucleotide
  source 1..328: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaagggagggtatgcactcaacttcggcaaaggcacctcgctgttggtcacacccc

SEQ ID NO: 491 - protein, 109 aa, synthetic construct
  REGION 1..109: note = Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVKGGYALNFGKGTSLLVTP

SEQ ID NO: 492 - protein, 276 aa, synthetic construct
  REGION 1..276: note = Description of Artificial Sequence: Synthetic polypeptide
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYSPPLPGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 493 - DNA, 346 bp, synthetic construct
  misc_feature 1..346: note = Description of Artificial Sequence: Synthetic polynucleotide
  source 1..346: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttatagtccccccctccccggtaaccagttctattttgggacagggacaagtttgacggtcattccaa

SEQ ID NO: 494 - protein, 115 aa, synthetic construct
  REGION 1..115: note = Description of Artificial Sequence: Synthetic polypeptide
AQTVTQSQPEMSVQEAETVTLSCTYDTSESDYYLFWYKQPPSRQMILVIRQEAYKQQNATENRFSVNFQKAAKSFSLKISDSQLGDAAMYFCAYSPPLPGNQFYFGTGTSLTVIP

SEQ ID NO: 495 - protein, 270 aa, synthetic construct
  REGION 1..270: note = Description of Artificial Sequence: Synthetic polypeptide
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAGEPDSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 496 - DNA, 334 bp, synthetic construct
  misc_feature 1..334: note = Description of Artificial Sequence: Synthetic polynucleotide
  source 1..334: mol_type = other DNA
gagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccggtgaacctgatagcaactatcagttaatctggggcgctgggaccaagctaattataaagccag

SEQ ID NO: 497 - protein, 111 aa, synthetic construct
  REGION 1..111: note = Description of Artificial Sequence: Synthetic polypeptide
ELKVEQNPLFLSMQEGKNYTIYCNYSTTSDRLYWYRQDPGKSLESLFVLLSNGAVKQEGRLMASLDTKARLSTLHITAAVHDLSATYFCAGEPDSNYQLIWGAGTKLIIKP

SEQ ID NO: 498 - protein, 275 aa, synthetic construct
  REGION 1..275: note = Description of Artificial Sequence: Synthetic polypeptide
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVGGAGGYQKVTFGTGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 499 - DNA, 337 bp, synthetic construct
  misc_feature 1..337: note = Description of Artificial Sequence: Synthetic polynucleotide
  source 1..337: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtggggggggccgggggttaccagaaagttacctttggaactggaacaaagctccaagtcatcccaa

SEQ ID NO: 500 - protein, 112 aa, synthetic construct
  REGION 1..112: note = Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVGGAGGYQKVTFGTGTKLQVIP

SEQ ID NO: 501 - protein, 282 aa, synthetic construct
  REGION 1..282: note = Description of Artificial Sequence: Synthetic polypeptide
MQCQAHGILQQMWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVRDPLSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 502 - DNA, 340 bp, synthetic construct
  misc_feature 1..340: note = Description of Artificial Sequence: Synthetic polynucleotide
  source 1..340: mol_type = other DNA
ggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgagagatcctctttctggtggctacaataagctgatttttggagcagggaccaggctggctgtacacccat

SEQ ID NO: 503 - protein, 113 aa, synthetic construct
  REGION 1..113: note = Description of Artificial Sequence: Synthetic polypeptide
GQSLEQPSEVTAVEGAIVQINCTYQTSGFYGLSWYQQHDGGAPTFLSYNALDGLEETGRFSSFLSRSDSYGYLLLQELQMKDSASYFCAVRDPLSGGYNKLIFGAGTRLAVHP

SEQ ID NO: 504 - protein, 269 aa, synthetic construct
  REGION 1..269: note = Description of Artificial Sequence: Synthetic polypeptide
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 505 - DNA, 334 bp, synthetic construct
  misc_feature 1..334: note = Description of Artificial Sequence: Synthetic polynucleotide
  source 1..334: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgtattcaggaggaggtgctgacggactcacctttggcaaagggactcatctaatcatccagccct

SEQ ID NO: 506 - protein, 111 aa, synthetic construct
  REGION 1..111: note = Description of Artificial Sequence: Synthetic polypeptide
DAKTTQPNSMESNEEEPVHLPCNHSTISGTDYIHWYRQLPSQGPEYVIHGLTSNVNNRMASLAIAEDRKSSTLILHRATLRDAAVYYCILYSGGGADGLTFGKGTHLIIQP

SEQ ID NO: 507 - protein, 284 aa, synthetic construct
  REGION 1..284: note = Description of Artificial Sequence: Synthetic polypeptide
MAQELGMQCQARGILQQMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAGLFGNTPLVFGKGTRLSVIANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 508 - DNA, 328 bp, synthetic construct
  misc_feature 1..328: note = Description of Artificial Sequence: Synthetic polynucleotide
  source 1..328: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctggcctcttcggaaacacacctcttgtctttggaaagggcacaagactttctgtgattgcaa

SEQ ID NO: 509 - protein, 109 aa, synthetic construct
  REGION 1..109: note = Description of Artificial Sequence: Synthetic polypeptide
GQNIDQPTEMTATEGAIVQINCTYQTSGFNGLFWYQQHAGEAPTFLSYNVLDGLEEKGRFSSFLSRSKGYSYLLLKELQMKDSASYLCAGLFGNTPLVFGKGTRLSVIA

SEQ ID NO: 510 - protein, 273 aa, synthetic construct
  REGION 1..273: note = Description of Artificial Sequence: Synthetic polypeptide
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYFNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 511 - DNA, 337 bp, synthetic construct
  misc_feature 1..337: note = Description of Artificial Sequence: Synthetic polynucleotide
  source 1..337: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttattttaacaccgacaagctcatctttgggactgggaccagattacaagtctttccaa

SEQ ID NO: 512 - protein, 112 aa, synthetic construct
  REGION 1..112: note = Description of Artificial Sequence: Synthetic polypeptide
AQTVTQSQPEMSVQEAETVTLSCTYDTSESDYYLFWYKQPPSRQMILVIRQEAYKQQNATENRFSVNFQKAAKSFSLKISDSQLGDAAMYFCAYFNTDKLIFGTGTRLQVFP

SEQ ID NO: 513 - protein, 275 aa, synthetic construct
  REGION 1..275: note = Description of Artificial Sequence: Synthetic polypeptide
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGGQGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 514 - DNA, 337 bp, synthetic construct
  misc_feature 1..337: note = Description of Artificial Sequence: Synthetic polynucleotide
  source 1..337: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatggggggccaaggaggtgctgacggactcacctttggcaaagggactcatctaatcatccagccct

SEQ ID NO: 515 - protein, 112 aa, synthetic construct
  REGION 1..112: note = Description of Artificial Sequence: Synthetic polypeptide
QKEVEQDPGPLSVPEGAIVSLNCTYSNSAFQYFMWYRQYSRKGPELLMYTYSSGNKEDGRFTAQVDKSSKYISLFIRDSQPSDSATYLCAMGGQGGADGLTFGKGTHLIIQP

SEQ ID NO: 516 - protein, 271 aa, synthetic construct
  REGION 1..271: note = Description of Artificial Sequence: Synthetic polypeptide
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPKREYGNKLVFGAGTILRVKSYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 517 - DNA, 337 bp, synthetic construct
  misc_feature 1..337: note = Description of Artificial Sequence: Synthetic polynucleotide
  source 1..337: mol_type = other DNA
ggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggccccaagagggaatatggaaacaagctggtctttggcgcaggaaccattctgagagtcaagtcct

SEQ ID NO: 518 - protein, 112 aa, synthetic construct
  REGION 1..112: note = Description of Artificial Sequence: Synthetic polypeptide
GQQLNQSPQSMFIQEGEDVSMNCTSSSIFNTWLWYKQDPGEGPVLLIALYKAGELTSNGRLTAQFGITRKDSFLNISASIPSDVGIYFCAGPKREYGNKLVFGAGTILRVKS

SEQ ID NO: 519 - protein, 272 aa, synthetic construct
  REGION 1..272: note = Description of Artificial Sequence: Synthetic polypeptide
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVEDNNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 520 - DNA, 319 bp, synthetic construct
  misc_feature 1..319: note = Description of Artificial Sequence: Synthetic polynucleotide
  source 1..319: mol_type = other DNA
aaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggaggataacaataacaatgacatgcgctttggagcagggaccagactgacagtaaaaccaa

SEQ ID NO: 521 - protein, 106 aa, synthetic construct
  REGION 1..106: note = Description of Artificial Sequence: Synthetic polypeptide
KDQVFQPSTVASSEGAVVEIFCNHSVSNAYNFFWYLHFPGCAPRLLVKGSKPSQQGRYNMTYERFSSSLLILQVREADAAVYYCAVEDNNNNDMRFGAGTRLTVKP

SEQ ID NO: 522 - protein, 277 aa, synthetic construct
  REGION 1..277: note = Description of Artificial Sequence: Synthetic polypeptide
MQCQAHGILQQMWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVNNNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 523 - DNA, 325 bp, synthetic construct
  misc_feature 1..325: note = Description of Artificial Sequence: Synthetic polynucleotide
  source 1..325: mol_type = other DNA
ggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtgaataacaatgccagactcatgtttggagatggaactcagctggtggtgaagccca

SEQ ID NO: 524 - protein, 108 aa, synthetic construct
  REGION 1..108: note = Description of Artificial Sequence: Synthetic polypeptide
GQSLEQPSEVTAVEGAIVQINCTYQTSGFYGLSWYQQHDGGAPTFLSYNALDGLEETGRFSSFLSRSDSYGYLLLQELQMKDSASYFCAVNNNARLMFGDGTQLVVKP

SEQ ID NO: 525 - protein, 284 aa, synthetic construct
  REGION 1..284: note = Description of Artificial Sequence: Synthetic polypeptide
MAQELGMQCQARGILQQMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRDPGNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 526 - DNA, 328 bp, synthetic construct
  misc_feature 1..328: note = Description of Artificial Sequence: Synthetic polynucleotide
  source 1..328: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagatccgggcaatgacatgcgctttggagcagggaccagactgacagtaaaaccaa

SEQ ID NO: 527 - protein, 109 aa, synthetic construct
  REGION 1..109: note = Description of Artificial Sequence: Synthetic polypeptide
GQNIDQPTEMTATEGAIVQINCTYQTSGFNGLFWYQQHAGEAPTFLSYNVLDGLEEKGRFSSFLSRSKGYSYLLLKELQMKDSASYLCAVRDPGNDMRFGAGTRLTVKP

SEQ ID NO: 528 - protein, 276 aa, synthetic construct
  REGION 1..276: note = Description of Artificial Sequence: Synthetic polypeptide
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFGGETSGSRLTFGEGTQLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 529 - DNA, 346 bp, synthetic construct
  misc_feature 1..346: note = Description of Artificial Sequence: Synthetic polynucleotide
  source 1..346: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcgggggagaaaccagtggctctaggttgacctttggggaaggaacacagctcacagtgaatcctg

SEQ ID NO: 530 - protein, 115 aa, synthetic construct
  REGION 1..115: note = Description of Artificial Sequence: Synthetic polypeptide
AQTVTQSQPEMSVQEAETVTLSCTYDTSENNYYLFWYKQPPSRQMILVIRQEAYKQQNATENRFSVNFQKAAKSFSLKISDSQLGDTAMYFCAFGGETSGSRLTFGEGTQLTVNP

SEQ ID NO: 531 - protein, 269 aa, synthetic construct
  REGION 1..269: note = Description of Artificial Sequence: Synthetic polypeptide
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDKGSNYQLIWGAGTKLIIKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 532 - DNA, 334 bp, synthetic construct
  misc_feature 1..334: note = Description of Artificial Sequence: Synthetic polynucleotide
  source 1..334: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgacaaaggaagcaactatcagttaatctggggcgctgggaccaagctaattataaagccag

SEQ ID NO: 533 - protein, 111 aa, synthetic construct
  REGION 1..111: note = Description of Artificial Sequence: Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVGDKGSNYQLIWGAGTKLIIKP

SEQ ID NO: 534 - protein, 270 aa, synthetic construct
  REGION 1..270: note = Description of Artificial Sequence: Synthetic polypeptide
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASYLPDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 535 - DNA, 331 bp, synthetic construct
  misc_feature 1..331: note = Description of Artificial Sequence: Synthetic polynucleotide
  source 1..331: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagttatctccctgacatgcgctttggagcagggaccagactgacagtaaaaccaa

SEQ ID NO: 536 - protein, 110 aa, synthetic construct
  REGION 1..110: note = Description of Artificial Sequence: Synthetic polypeptide
GENVEQHPSTLSVQEGDSAVIKCTYSDSASNYFPWYKQELGKRPQLIIDIRSNVGEKKDQRIAVTLNKTAKHFSLHITETQPEDSAVYFCAASYLPDMRFGAGTRLTVKP

SEQ ID NO: 537 - protein, 279 aa, synthetic construct
  REGION 1..279: note = Description of Artificial Sequence: Synthetic polypeptide
MNMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSELKAAGNKLTFGGGTRVLVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 538 - DNA, 349 bp, synthetic construct
  misc_feature 1..349: note = Description of Artificial Sequence: Synthetic polynucleotide
  source 1..349: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagctcaaagctgcaggcaacaagctaacttttggaggaggaaccagggtgctagttaaaccaa

SEQ ID NO: 539 - protein, 116 aa, synthetic construct
  REGION 1..116: note = Description of Artificial Sequence: Synthetic polypeptide
AQKVTQAQTEISVVEKEDVTLDCVYETRDTTYYLFWYKQPPSGELVFLIRRNSFDEQNEISGRYSWNFQKSTSSFNFTITASQVVDSAVYFCALSELKAAGNKLTFGGGTRVLVKP

SEQ ID NO: 540 - protein, 272 aa, synthetic construct
  REGION 1..272: note = Description of Artificial Sequence: Synthetic polypeptide
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDKVYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 541 - DNA, 343 bp, synthetic construct
  misc_feature 1..343: note = Description of Artificial Sequence: Synthetic polynucleotide
  source 1..343: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtgacaaggtttatggaggaagccaaggaaatctcatctttggaaaaggcactaaactctctgttaaaccaa

SEQ ID NO: 542 - protein, 114 aa, synthetic construct
  REGION 1..114: note = Description of Artificial Sequence: Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVGDKVYGGSQGNLIFGKGTKLSVKP

SEQ ID NO: 543 - protein, 277 aa, synthetic construct
  REGION 1..277: note = Description of Artificial Sequence: Synthetic polypeptide
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGQDSSYKLIFGSGTRLLVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 544 - DNA, 349 bp, synthetic construct
  misc_feature 1..349: note = Description of Artificial Sequence: Synthetic polynucleotide
  source 1..349: mol_type = other DNA
gcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtatttctgtgcaatgagagagggccaggatagcagctataaattgatcttcgggagtgggaccagactgctggtcaggcctg

SEQ ID NO: 545 - protein, 116 aa, synthetic construct
  REGION 1..116: note = Description of Artificial Sequence: Synthetic polypeptide
AQKITQTQPGMFVQEKEAVTLDCTYDTSDPSYGLFWYKQPSSGEMIFLIYQGSYDQQNATEGRYSLNFQKARKSANLVISASQLGDSAMYFCAMREGQDSSYKLIFGSGTRLLVRP

SEQ ID NO: 546 - protein, 281 aa, synthetic construct
  REGION 1..281: note = Description of Artificial Sequence: Synthetic polypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARIYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 547 - DNA, 343 bp, synthetic construct
  misc_feature 1..343: note = Description of Artificial Sequence: Synthetic polynucleotide
  source 1..343: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaaggatttatggaggaagccaaggaaatctcatctttggaaaaggcactaaactctctgttaaaccaa

SEQ ID NO: 548 - protein, 114 aa, synthetic construct
  REGION 1..114: note = Description of Artificial Sequence: Synthetic polypeptide
DQQVKQNSPSLSVQEGRISILNCDYTNSMFDYFLWYKKYPAEGPTFLISISSIKDKNEDGRFTVFLNKSAKHLSLHIVPSQPGDSAVYFCAARIYGGSQGNLIFGKGTKLSVKP

SEQ ID NO: 549 - protein, 279 aa, synthetic construct
  REGION 1..279: note = Description of Artificial Sequence: Synthetic polypeptide
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMREGEPSGSARQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 550 - DNA, 355 bp, synthetic construct
  misc_feature 1..355: note = Description of Artificial Sequence: Synthetic polynucleotide
  source 1..355: mol_type = other DNA
gcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtacttctgtgcaatgagagagggcgaaccttctggttctgcaaggcaactgacctttggatctgggacacaattgactgttttacctg

SEQ ID NO: 551 - protein, 118 aa, synthetic construct
  REGION 1..118: note = Description of Artificial Sequence: Synthetic polypeptide
AQKITQTQPGMFVQEKEAVTLDCTYDTSDPSYGLFWYKQPSSGEMIFLIYQGSYDQQNATEGRYSLNFQKARKSANLVISASQLGDSAMYFCAMREGEPSGSARQLTFGSGTQLTVLP

SEQ ID NO: 552 - protein, 281 aa, synthetic construct
  REGION 1..281: note = Description of Artificial Sequence: Synthetic polypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAVNAGGTSYGKLTFGQGTILTVHPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 553 - DNA, 343 bp, synthetic construct
  misc_feature 1..343: note = Description of Artificial Sequence: Synthetic polynucleotide
  source 1..343: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagttaatgctggtggtactagctatggaaagctgacatttggacaagggaccatcttgactgtccatccaa

SEQ ID NO: 554 - protein, 114 aa, synthetic construct
  REGION 1..114: note = Description of Artificial Sequence: Synthetic polypeptide
DQQVKQNSPSLSVQEGRISILNCDYTNSMFDYFLWYKKYPAEGPTFLISISSIKDKNEDGRFTVFLNKSAKHLSLHIVPSQPGDSAVYFCAVNAGGTSYGKLTFGQGTILTVHP

SEQ ID NO: 555 - protein, 276 aa, synthetic construct
  REGION 1..276: note = Description of Artificial Sequence: Synthetic polypeptide
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSEKFSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 556 - DNA, 349 bp, synthetic construct
  misc_feature 1..349: note = Description of Artificial Sequence: Synthetic polynucleotide
  source 1..349: mol_type = other DNA
gcccagtcggtgacccagcttggcagccacgtctctgtctctgagggagccctggttctgctgaggtgcaactactcatcgtctgttccaccatatctcttctggtatgtgcaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgagtgagaagttttctggtggctacaataagctgatttttggagcagggaccaggctggctgtacacccat

SEQ ID NO: 557 - protein, 116 aa, synthetic construct
  REGION 1..116: note = Description of Artificial Sequence: Synthetic polypeptide
AQSVTQLGSHVSVSEGALVLLRCNYSSSVPPYLFWYVQYPNQGLQLLLKYTTGATLVKGINGFEAEFKKSETSFHLTKPSAHMSDAAEYFCAVSEKFSGGYNKLIFGAGTRLAVHP

SEQ ID NO: 558 - protein, 277 aa, synthetic construct
  REGION 1..277: note = Description of Artificial Sequence: Synthetic polypeptide
MNMLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEAKDDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 559 - DNA, 343 bp, synthetic construct
  misc_feature 1..343: note = Description of Artificial Sequence: Synthetic polynucleotide
  source 1..343: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcaaaagatgacaagatcatctttggaaaagggacacgacttcatattctcccca

SEQ ID NO: 560 - protein, 114 aa, synthetic construct
  REGION 1..114: note = Description of Artificial Sequence: Synthetic polypeptide
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEAKDDKIIFGKGTRLHILP

SEQ ID NO: 561 - protein, 278 aa, synthetic construct
  REGION 1..278: note = Description of Artificial Sequence: Synthetic polypeptide
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAIYNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 562 - DNA, 334 bp, synthetic construct
  misc_feature 1..334: note = Description of Artificial Sequence: Synthetic polynucleotide
  source 1..334: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgccatttataaccagggaggaaagcttatcttcggacagggaacggagttatctgtgaaaccca

SEQ ID NO: 563 - protein, 111 aa, synthetic construct
  REGION 1..111: note = Description of Artificial Sequence: Synthetic polypeptide
DQQVKQNSPSLSVQEGRISILNCDYTNSMFDYFLWYKKYPAEGPTFLISISSIKDKNEDGRFTVFLNKSAKHLSLHIVPSQPGDSAVYFCAIYNQGGKLIFGQGTELSVKP

SEQ ID NO: 564 - protein, 270 aa, synthetic construct
  REGION 1..270: note = Description of Artificial Sequence: Synthetic polypeptide
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALMTDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 565 - DNA, 331 bp, synthetic construct
  misc_feature 1..331: note = Description of Artificial Sequence: Synthetic polynucleotide
  source 1..331: mol_type = other DNA
ggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgatgaccgactacaagctcagctttggagccggaaccacagtaactgtaagagcaa

SEQ ID NO: 566 - protein, 110 aa, synthetic construct
  REGION 1..110: note = Description of Artificial Sequence: Synthetic polypeptide
GNSVTQMEGPVTLSEEAFLTINCTYTATGYPSLFWYVQYPGEGLQLLLKATKADDKGSNKGFEATYRKETTSFHLEKGSVQVSDSAVYFCALMTDYKLSFGAGTTVTVRA

SEQ ID NO: 567 - protein, 278 aa, synthetic construct
  REGION 1..278: note = Description of Artificial Sequence: Synthetic polypeptide
MMKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVRSVGVSGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 568 - DNA, 346 bp, synthetic construct
  misc_feature 1..346: note = Description of Artificial Sequence: Synthetic polynucleotide
  source 1..346: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaggagtgtaggggtttctggtggctacaataagctgatttttggagcagggaccaggctggctgtacacccat

SEQ ID NO: 569 - protein, 115 aa, synthetic construct
  REGION 1..115: note = Description of Artificial Sequence: Synthetic polypeptide
QKEVEQNSGPLSVPEGAIASLNCTYSDRGSQSFFWYRQYSGKSPELIMSIYSNGDKEDGRFTAQLNKASQYVSLLIRDSQPSDSATYLCAVRSVGVSGGYNKLIFGAGTRLAVHP

SEQ ID NO: 570 - protein, 269 aa, synthetic construct
  REGION 1..269: note = Description of Artificial Sequence: Synthetic polypeptide
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVLLSSNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 571 - DNA, 334 bp, synthetic construct
  misc_feature 1..334: note = Description of Artificial Sequence: Synthetic polynucleotide
  source 1..334: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgttctactctctagcaacacaggcaaactaatctttgggcaagggacaactttacaagtaaaaccag

SEQ ID NO: 572 - protein, 111 aa, synthetic construct
  REGION 1..111: note = Description of Artificial Sequence: Synthetic polypeptide
LAKTTQPISMDSYEGQEVNITCSHNNIATNDYITWYQQFPSQGPRFIIQGYKTKVTNEVASLFIPADRKSSTLSLPRVSLSDTAVYYCLVLLSSNTGKLIFGQGTTLQVKP

SEQ ID NO: 573 - protein, 275 aa, synthetic construct
  REGION 1..275: note = Description of Artificial Sequence: Synthetic polypeptide
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSANNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 574 - DNA, 343 bp, synthetic construct
  misc_feature 1..343: note = Description of Artificial Sequence: Synthetic polynucleotide
  source 1..343: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgcgaataacaatgacatgcgctttggagcagggaccagactgacagtaaaaccaa

SEQ ID NO: 575 - protein, 114 aa, synthetic construct
  REGION 1..114: note = Description of Artificial Sequence: Synthetic polypeptide
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSANNNDMRFGAGTRLTVKP

SEQ ID NO: 576 - protein, 273 aa, synthetic construct
  REGION 1..273: note = Description of Artificial Sequence: Synthetic polypeptide
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVFPGGSYIPTFGRGTSLIVHPYIQKPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 577 - DNA, 331 bp, synthetic construct
  misc_feature 1..331: note = Description of Artificial Sequence: Synthetic polynucleotide
  source 1..331: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggttttcccaggaggaagctacatacctacatttggaagaggaaccagccttattgttcatccgt

SEQ ID NO: 578 - protein, 110 aa, synthetic construct
  REGION 1..110: note = Description of Artificial Sequence: Synthetic polypeptide
RKEVEQDPGPFNVPEGATVAFNCTYSNSASQSFFWYRQDCRKEPKLLMSVYSSGNEDGRFTAQLNRASQYISLLIRDSKLSDSATYLCVVFPGGSYIPTFGRGTSLIVHP

SEQ ID NO: 579 - protein, 315 aa, synthetic construct
  REGION 1..315: note = Description of Artificial Sequence: Synthetic polypeptide
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGLALTEGGWYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 580 - DNA, 352 bp, synthetic construct
  misc_feature 1..352: note = Description of Artificial Sequence: Synthetic polynucleotide
  source 1..352: mol_type = other DNA
gatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggtttggccctcactgaggggggctggtacgagcagtacttcgggccgggcaccaggctcacggtcacag

SEQ ID NO: 581 - protein, 117 aa, synthetic construct
  REGION 1..117: note = Description of Artificial Sequence: Synthetic polypeptide
DARVTQTPRHKVTEMGQEVTMRCQPILGHNTVFWYRQTMMQGLELLAYFRNRAPLDDSGMPKDRFSAEMPDATLATLKIQPSEPRDSAVYFCASGLALTEGGWYEQYFGPGTRLTVT

SEQ ID NO: 582 - protein, 312 aa, synthetic construct
  REGION 1..312: note = Description of Artificial Sequence: Synthetic polypeptide
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASLRWDGDNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 583 - DNA, 343 bp, synthetic construct
  misc_feature 1..343: note = Description of Artificial Sequence: Synthetic polynucleotide
  source 1..343: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcctccgatgggacggggacaatgagcagttcttcgggccagggacacggctcaccgtgctag

SEQ ID NO: 584 - protein, 114 aa, synthetic construct
  REGION 1..114: note = Description of Artificial Sequence: Synthetic polypeptide
EPEVTQTPSHQVTQMGQEVILRCVPISNHLYFYWYRQILGQKVEFLVSFYNNEISEKSEIFDDQFSVERPDGSNFTLKIRSTKLEDSAMYFCASLRWDGDNEQFFGPGTRLTVL

SEQ ID NO: 585 - protein, 339 aa, synthetic construct
  REGION 1..339: note = Description of Artificial Sequence: Synthetic polypeptide
MKSQNDPLESTVPLSPMHRPRRPLHPVAPAMSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRDRALNTEAFFGQGTRLTVVEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 586 - DNA, 340 bp, synthetic construct
  misc_feature 1..340: note = Description of Artificial Sequence: Synthetic polynucleotide
  source 1..340: mol_type = other DNA
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagggacagggccctgaacactgaagctttctttggacaaggcaccagactcacagttgtag

SEQ ID NO: 587 - protein, 113 aa, synthetic construct
  REGION 1..113: note = Description of Artificial Sequence: Synthetic polypeptide
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASRDRALNTEAFFGQGTRLTVV

SEQ ID NO: 588 - protein, 358 aa, synthetic construct
  REGION 1..358: note = Description of Artificial Sequence: Synthetic polypeptide
MGVASPGLSLPGAQAGRMSQNDFLESPAPLSSMHRYRRPLRHAASAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNGGTLIYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 589 - DNA, 343 bp, synthetic construct
  misc_feature 1..343: note = Description of Artificial Sequence: Synthetic polynucleotide
  source 1..343: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagaaacggcgggacactaatctacgagcagtacttcgggccgggcaccaggctcacggtcacag

SEQ ID NO: 590 - protein, 114 aa, synthetic construct
  REGION 1..114: note = Description of Artificial Sequence: Synthetic polypeptide
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRNGGTLIYEQYFGPGTRLTVT

SEQ ID NO: 591 - protein, 308 aa, synthetic construct
  REGION 1..308: note = Description of Artificial Sequence: Synthetic polypeptide
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASRQRTELEAFFGQGTRLTVVEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 592 - DNA, 337 bp, synthetic construct
  misc_feature 1..337: note = Description of Artificial Sequence: Synthetic polynucleotide
  source 1..337: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcaggcagcggacagaacttgaagctttctttggacaaggcaccagactcacagttgtag

SEQ ID NO: 593 - protein, 112 aa, synthetic construct
  REGION 1..112: note = Description of Artificial Sequence: Synthetic polypeptide
KAGVTQTPRYLIKTRGQQVTLSCSPISGHRSVSWYQQTPGQGLQFLFEYFSETQRNKGNFPGRFSGRQFSNSRSEMNVSTLELGDSALYLCASRQRTELEAFFGQGTRLTVV

SEQ ID NO: 594 - protein, 314 aa, synthetic construct
  REGION 1..314: note = Description of Artificial Sequence: Synthetic polypeptide
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAPDRGTYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 595 - DNA, 349 bp, synthetic construct
  misc_feature 1..349: note = Description of Artificial Sequence: Synthetic polynucleotide
  source 1..349: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagcccctgacaggggtacctacgagcagtacttcgggccgggcaccaggctcacggtcacag

SEQ ID NO: 596 - protein, 116 aa, synthetic construct
  REGION 1..116: note = Description of Artificial Sequence: Synthetic polypeptide
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSEAPDRGTYEQYFGPGTRLTVT

SEQ ID NO: 597 - protein, 312 aa, synthetic construct
  REGION 1..312: note = Description of Artificial Sequence: Synthetic polypeptide
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSELGRGFYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 598 - DNA, 343 bp, synthetic construct
  misc_feature 1..343: note = Description of Artificial Sequence: Synthetic polynucleotide
  source 1..343: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctacgttgtctccagatccaagacagagaatttccccctcactctggagtcagctacccgctcccagacatctgtgtatttctgcgccagcagtgagttgggcaggggtttctacgagcagtacttcgggccgggcaccaggctcacggtcacag

SEQ ID NO: 599 - protein, 114 aa, synthetic construct
  REGION 1..114: note = Description of Artificial Sequence: Synthetic polypeptide
DAGITQSPRYKITETGRQVTLMCHQTWSHSYMFWYRQDLGHGLRLIYYSAAADITDKGEVPDGYVVSRSKTENFPLTLESATRSQTSVYFCASSELGRGFYEQYFGPGTRLTVT

SEQ ID NO: 600 - protein, 359 aa, synthetic construct
  REGION 1..359: note = Description of Artificial Sequence: Synthetic polypeptide
MGVASPGLSLPGAQAGRMSQNDFLESPAPLSSMHRYRRPLRHAASAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSHLGAGGPHEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 601 - DNA, 346 bp, synthetic construct
  misc_feature 1..346: note = Description of Artificial Sequence: Synthetic polynucleotide
  source 1..346: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagccatctgggggcgggagggccgcacgagcagtacttcgggccgggcaccaggctcacggtcacag

SEQ ID NO: 602 - protein, 115 aa, synthetic construct
  REGION 1..115: note = Description of Artificial Sequence: Synthetic polypeptide
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSHLGAGGPHEQYFGPGTRLTVT

SEQ ID NO: 603 - protein, 312 aa, synthetic construct
  REGION 1..312: note = Description of Artificial Sequence: Synthetic polypeptide
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSHTDGSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 604 - DNA, 343 bp, synthetic construct
  misc_feature 1..343: note = Description of Artificial Sequence: Synthetic polynucleotide
  source 1..343: mol_type = other DNA
ggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagccatactgacggctcctacgagcagtacttcgggccgggcaccaggctcacggtcacag

SEQ ID NO: 605 - protein, 114 aa, synthetic construct
  REGION 1..114: note = Description of Artificial Sequence: Synthetic polypeptide
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSHTDGSYEQYFGPGTRLTVT

SEQ ID NO: 606 - protein, 209 aa, synthetic construct
  REGION 1..209: note = Description of Artificial Sequence: Synthetic polypeptide
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGAFAGQPQHFGDGTRLSILEDLNKVFPPEVAVFEPSEAEISHTQKATLVCLATGFYPDHVELSWWVNGKEVHSGVSTDPQPLKEQPALNDSRYCL

SEQ ID NO: 607 - DNA, 343 bp, synthetic construct
  misc_feature 1..343: note = Description of Artificial Sequence: Synthetic polynucleotide
  source 1..343: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcataggggcatttgctggtcagccccagcattttggtgatgggactcgactctccatcctag

SEQ ID NO: 608 - protein, 114 aa, synthetic construct
  REGION 1..114: note = Description of Artificial Sequence: Synthetic polypeptide
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIGAFAGQPQHFGDGTRLSIL

SEQ ID NO: 609 - protein, 316 aa, synthetic construct
  REGION 1..316: note = Description of Artificial Sequence: Synthetic polypeptide
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLAPTPGPDTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 610 - DNA, 355 bp, synthetic construct
  misc_feature 1..355: note = Description of Artificial Sequence: Synthetic polynucleotide
  source 1..355: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagcgcctacccccgggccggacaccggggagctgttttttggagaaggctctaggctgaccgtactgg

SEQ ID NO: 611 - protein, 118 aa, synthetic construct
  REGION 1..118: note = Description of Artificial Sequence: Synthetic polypeptide
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLAPTPGPDTGELFFGEGSRLTVL

SEQ ID NO: 612 - protein, 310 aa, synthetic construct
  REGION 1..310: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGNIYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 613 - DNA, 337 bp, synthetic construct
  misc_feature 1..337: note = Description of Artificial Sequence: Synthetic polynucleotide
  source 1..337: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttagggaatatctacgagcagtacttcgggccgggcaccaggctcacggtcacag

SEQ ID NO: 614 - protein, 112 aa, synthetic construct
  REGION 1..112: note = Description of Artificial Sequence: Synthetic polypeptide
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGNIYEQYFGPGTRLTVT

SEQ ID NO: 615 - protein, 310 aa, synthetic construct
  REGION 1..310: note = Description of Artificial Sequence: Synthetic polypeptide
MGTRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLGSGEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 616 - DNA, 337 bp, synthetic construct
  misc_feature 1..337: note = Description of Artificial Sequence: Synthetic polynucleotide
  source 1..337: mol_type = other DNA
ggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttagggagcggtgagcagttcttcgggccagggacacggctcaccgtgctag

SEQ ID NO: 617 - protein, 112 aa, synthetic construct
  REGION 1..112: note = Description of Artificial Sequence: Synthetic polypeptide
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSLGSGEQFFGPGTRLTVL

SEQ ID NO: 618 - protein, 310 aa, synthetic construct
  REGION 1..310: note = Description of Artificial Sequence: Synthetic polypeptide
MGPRLLFWALLCLLGTGPVEAGVTQSPTHLIKTRGQQATLRCSPISGHTSVYWYQQALGLGLQFLLWYDEGEERNRGNFPPRFSGRQFPNYSSELNVNALELEDSALYLCASSLQTSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 619 - DNA, 337 bp, synthetic construct
  misc_feature 1..337: note = Description of Artificial Sequence: Synthetic polynucleotide
  source 1..337: mol_type = other DNA
gaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagcttacagacatcctacgagcagtacttcgggccgggcaccaggctcacggtcacag

SEQ ID NO: 620 - protein, 112 aa, synthetic construct
  REGION 1..112: note = Description of Artificial Sequence: Synthetic polypeptide
EAGVTQSPTHLIKTRGQQATLRCSPISGHTSVYWYQQALGLGLQFLLWYDEGEERNRGNFPPRFSGRQFPNYSSELNVNALELEDSALYLCASSLQTSYEQYFGPGTRLTVT

SEQ ID NO: 621 - protein, 315 aa, synthetic construct
  REGION 1..315: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSWGTGKRADTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 622 - DNA, 352 bp, synthetic construct
  misc_feature 1..352: note = Description of Artificial Sequence: Synthetic polynucleotide
  source 1..352: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcgtgggggaccggcaaaagagcagatacgcagtattttggcccaggcacccggctgacagtgctcg

SEQ ID NO: 623 - protein, 117 aa, synthetic construct
  REGION 1..117: note = Description of Artificial Sequence: Synthetic polypeptide
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSWGTGKRADTQYFGPGTRLTVL

SEQ ID NO: 624 - protein, 313 aa, synthetic construct
  REGION 1..313: note = Description of Artificial Sequence: Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVGGGSNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 625 - DNA, 346 bp, synthetic construct
  misc_feature 1..346: note = Description of Artificial Sequence: Synthetic polynucleotide
  source 1..346: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagttggcggcgggagcaatgagcagttcttcgggccagggacacggctcaccgtgctag

SEQ ID NO: 626 - protein, 115 aa, synthetic construct
  REGION 1..115: note = Description of Artificial Sequence: Synthetic polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVGGGSNEQFFGPGTRLTVL

SEQ ID NO: 627 - protein, 309 aa, synthetic construct
  REGION 1..309: note = Description of Artificial Sequence: Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVGGTEAFFGQGTRLTVVEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 628 - DNA, 340 bp, synthetic construct
  misc_feature 1..340: note = Description of Artificial Sequence: Synthetic polynucleotide
  source 1..340: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagtcggaggcactgaagctttctttggacaaggcaccagactcacagttgtag

SEQ ID NO: 629 - protein, 113 aa, synthetic construct
  REGION 1..113: note = Description of Artificial Sequence: Synthetic polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVGGTEAFFGQGTRLTVV

SEQ ID NO: 630 - protein, 311 aa, synthetic construct
  REGION 1..311: note = Description of Artificial Sequence: Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPDRNLGQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 631 - DNA, 340 bp, synthetic construct
  misc_feature 1..340: note = Description of Artificial Sequence: Synthetic polynucleotide
  source 1..340: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccgatcggaatctcgggcagtacttcgggccgggcaccaggctcacggtcacag

SEQ ID NO: 632 - protein, 113 aa, synthetic construct
  REGION 1..113: note = Description of Artificial Sequence: Synthetic polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPDRNLGQYFGPGTRLTVT

SEQ ID NO: 633 - protein, 312 aa, synthetic construct
  REGION 1..312: note = Description of Artificial Sequence: Synthetic polypeptide
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQVQAFNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 634 - DNA, 343 bp, synthetic construct
  misc_feature 1..343: note = Description of Artificial Sequence: Synthetic polynucleotide
  source 1..343: mol_type = other DNA
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgaatccggtatgcccaacaatcgattcttagctgaaaggactggagggacgtattctactctgaaggtgcagcctgcagaactggaggattctggagtttatttctgtgccagcagccaagtccaggcttttaatgagcagttcttcgggccagggacacggctcaccgtgctag

SEQ ID NO: 635 - protein, 114 aa, synthetic construct
  REGION 1..114: note = Description of Artificial Sequence: Synthetic polypeptide
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQVQAFNEQFFGPGTRLTVL

SEQ ID NO: 636 - protein, 344 aa, synthetic construct
  REGION 1..344: note = Description of Artificial Sequence: Synthetic polypeptide
MRGQGRPSSSAFAHSDPDWAKLPSFPDPAMGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSRGEPGSGANVLTFGAGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 637 - DNA, 352 bp, synthetic construct
  misc_feature 1..352: note = Description of Artificial Sequence: Synthetic polynucleotide
  source 1..352: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagtcgaggggagccaggctctggggccaacgtcctgactttcggggccggcagcaggctgaccgtgctgg

SEQ ID NO: 638 - protein, 117 aa, synthetic construct
  REGION 1..117: note = Description of Artificial Sequence: Synthetic polypeptide
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSRGEPGSGANVLTFGAGSRLTVL

SEQ ID NO: 639 - protein, 323 aa, synthetic construct
  REGION 1..323: note = Description of Artificial Sequence: Synthetic polypeptide
MQCFLSLCAMGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSSGTDPSGANVLTFGAGSRLTVLEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 640 - DNA, 349 bp, synthetic construct
  misc_feature 1..349: note = Description of Artificial Sequence: Synthetic polynucleotide
  source 1..349: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctccgggacagacccctctggggccaacgtcctgactttcggggccggcagcaggctgaccgtgctgg

SEQ ID NO: 641 - protein, 116 aa, synthetic construct
  REGION 1..116: note = Description of Artificial Sequence: Synthetic polypeptide
DAGVTQSPTHLIKTRGQQVTLRCSPKSGHDTVSWYQQALGQGPQFIFQYYEEEERQRGNFPDRFSGHQFPNYSSELNVNALLLGDSALYLCASSSGTDPSGANVLTFGAGSRLTVL

SEQ ID NO: 642 - protein, 312 aa, synthetic construct
  REGION 1..312: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTGQNIGGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 643 - DNA, 343 bp, synthetic construct
  misc_feature 1..343: note = Description of Artificial Sequence: Synthetic polynucleotide
  source 1..343: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtaccggacagaatataggcggggagctgttttttggagaaggctctaggctgaccgtactgg

SEQ ID NO: 644 - protein, 114 aa, synthetic construct
  REGION 1..114: note = Description of Artificial Sequence: Synthetic polypeptide
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSTGQNIGGELFFGEGSRLTVL

SEQ ID NO: 645 - protein, 372 aa, synthetic construct
  REGION 1..372: note = Description of Artificial Sequence: Synthetic polypeptide
MVHIRVAAILDLTHQYQVILRLSDVTVGTALWRQGRPSSSAPAHSDPDLVKLPSCPDPAMGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSTHEKTGWKSPLHFGNGTRLTVTEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 646 - DNA, 352 bp, synthetic construct
  misc_feature 1..352: note = Description of Artificial Sequence: Synthetic polynucleotide
  source 1..352: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcacacatgaaaagacagggtggaaatcacccctccactttgggaatgggaccaggctcactgtgacag

SEQ ID NO: 647 - protein, 117 aa, synthetic construct
  REGION 1..117: note = Description of Artificial Sequence: Synthetic polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSTHEKTGWKSPLHFGNGTRLTVT

SEQ ID NO: 648 - protein, 313 aa, synthetic construct
  REGION 1..313: note = Description of Artificial Sequence: Synthetic polypeptide
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSTHSDRNLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 649 - DNA, 352 bp, synthetic construct
  misc_feature 1..352: note = Description of Artificial Sequence: Synthetic polynucleotide
  source 1..352: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtacgcactctgacaggaacttgaacactgaagctttctttggacaaggcaccagactcacagttgtag

SEQ ID NO: 650 - protein, 117 aa, synthetic construct
  REGION 1..117: note = Description of Artificial Sequence: Synthetic polypeptide
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSTHSDRNLNTEAFFGQGTRLTVV

SEQ ID NO: 651 - protein, 310 aa, synthetic construct
  REGION 1..310: note = Description of Artificial Sequence: Synthetic polypeptide
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSVQATGHGYTFGSGTRLTVVEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 652 - DNA, 343 bp, synthetic construct
  misc_feature 1..343: note = Description of Artificial Sequence: Synthetic polynucleotide
  source 1..343: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagcgtacaggctacgggccatggctacaccttcggttcggggaccaggttaaccgttgtag

SEQ ID NO: 653 - protein, 114 aa, synthetic construct
  REGION 1..114: note = Description of Artificial Sequence: Synthetic polypeptide
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSVQATGHGYTFGSGTRLTVV

SEQ ID NO: 654 - protein, 311 aa, synthetic construct
  REGION 1..311: note = Description of Artificial Sequence: Synthetic polypeptide
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASTPSGYNSWEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 655 - DNA, 346 bp, synthetic construct
  misc_feature 1..346: note = Description of Artificial Sequence: Synthetic polynucleotide
  source 1..346: mol_type = other DNA
ggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcaccccctctggctataactcttgggagcagttcttcgggccagggacacggctcaccgtgctag

SEQ ID NO: 656 - protein, 115 aa, synthetic construct
  REGION 1..115: note = Description of Artificial Sequence: Synthetic polypeptide
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASTPSGYNSWEQFFGPGTRLTVL

SEQ ID NO: 657 - protein, 309 aa, synthetic construct
  REGION 1..309: note = Description of Artificial Sequence: Synthetic polypeptide
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCATAPRGSNQPQHFGDGTRLSILEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 658 - DNA, 340 bp, synthetic construct
  misc_feature 1..340: note = Description of Artificial Sequence: Synthetic polynucleotide
  source 1..340: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccaccgcccccaggggtagcaatcagccccagcattttggtgatgggactcgactctccatcctag

SEQ ID NO: 659 - protein, 113 aa, synthetic construct
  REGION 1..113: note = Description of Artificial Sequence: Synthetic polypeptide
EAQVTQNPRYLITVTGKKLTVTCSQNMNHEYMSWYRQDPGLGLRQIYYSMNVEVTDKGDVPEGYKVSRKEKRNFPLILESPSPNQTSLYFCATAPRGSNQPQHFGDGTRLSIL

SEQ ID NO: 660 - protein, 326 aa, synthetic construct
  REGION 1..326: note = Description of Artificial Sequence: Synthetic polypeptide
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICRAWGGSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 661 - DNA, 349 bp, synthetic construct
  misc_feature 1..349: note = Description of Artificial Sequence: Synthetic polynucleotide
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagggcctggggcgggagctcctacaatgagcagttcttcgggccagggacacggctcaccgtgctag

SEQ ID NO: 662 - protein, 116 aa, synthetic construct
  REGION 1..116: note = Description of Artificial Sequence: Synthetic polypeptide
GAVVSQHPSWVICKSGTSVKIECRSLDFQATTMFWYRQFPKQSLMLMATSNEGSKATYEQGVEKDKFLINHASLTLSTLTVTSAHPEDSSFYICRAWGGSSYNEQFFGPGTRLTVL

SEQ ID NO: 663 - protein, 329 aa, synthetic construct
  REGION 1..329: note = Description of Artificial Sequence: Synthetic polypeptide
MEAVVTTLPREGGVRPSRKMLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARIGQGFMNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 664 - DNA, 352 bp, synthetic construct
  misc_feature 1..352: note = Description of Artificial Sequence: Synthetic polynucleotide
  source 1..352: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaattggacagggttttatgaatgagcagttcttcgggccagggacacggctcaccgtgctag

SEQ ID NO: 665 - protein, 117 aa, synthetic construct
  REGION 1..117: note = Description of Artificial Sequence: Synthetic polypeptide
GAVVSQHPSRVICKSGTSVKIECRSLDFQATTMFWYRQFPKQSLMLMATSNEGSKATYEQGVEKDKFLINHASLTLSTLTVTSAHPEDSSFYICSARIGQGFMNEQFFGPGTRLTVL

SEQ ID NO: 666 - protein, 325 aa, synthetic construct
  REGION 1..325: note = Description of Artificial Sequence: Synthetic polypeptide
MEAVVTTLPREGGVRPSRKMLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSAPGWRGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 667 - DNA, 346 bp, synthetic construct
  misc_feature 1..346: note = Description of Artificial Sequence: Synthetic polynucleotide
  source 1..346: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcctgggtggcgtggcactgaagctttctttggacaaggcaccagactcacagttgtag

SEQ ID NO: 668 - protein, 115 aa, synthetic construct
  REGION 1..115: note = Description of Artificial Sequence: Synthetic polypeptide
GAVVSQHPSRVICKSGTSVKIECRSLDFQATTMFWYRQFPKKSLMLMATSNEGSKATYEQGVEKDKFLINHASLTLSTLTVTSAHPEDSSFYICSAPGWRGTEAFFGQGTRLTVV

SEQ ID NO: 669 - protein, 309 aa, synthetic construct
  REGION 1..309: note = Description of Artificial Sequence: Synthetic polypeptide
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQDGLEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 670 - DNA, 334 bp, synthetic construct
  misc_feature 1..334: note = Description of Artificial Sequence: Synthetic polynucleotide
  source 1..334: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagatggacttgagcagtacttcgggccgggcaccaggctcacggtcacag

SEQ ID NO: 671 - protein, 111 aa, synthetic construct
  REGION 1..111: note = Description of Artificial Sequence: Synthetic polypeptide
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQDGLEQYFGPGTRLTVT

SEQ ID NO: 672 - protein, 318 aa, synthetic construct
  REGION 1..318: note = Description of Artificial Sequence: Synthetic polypeptide
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQEGSQDHTSGRATEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 673 - DNA, 361 bp, synthetic construct
  misc_feature 1..361: note = Description of Artificial Sequence: Synthetic polynucleotide
  source 1..361: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagaaggatctcaggaccacactagcgggagggccaccgagcagtacttcgggccgggcaccaggctcacggtcacag

SEQ ID NO: 674 - protein, 120 aa, synthetic construct
  REGION 1..120: note = Description of Artificial Sequence: Synthetic polypeptide
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQEGSQDHTSGRATEQYFGPGTRLTVT

SEQ ID NO: 675 - protein, 340 aa, synthetic construct
  REGION 1..340: note = Description of Artificial Sequence: Synthetic polypeptide
MSQNDFLESPAPLSSMHRYRRPLRHAASAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLTEGRSEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 676 - DNA, 340 bp, synthetic construct
  misc_feature 1..340: note = Description of Artificial Sequence: Synthetic polynucleotide
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagcctgacagaaggaagaagcgagcagtacttcgggccgggcaccaggctcacggtcacag

SEQ ID NO: 677 - protein, 113 aa, synthetic construct
  REGION 1..113: note = Description of Artificial Sequence: Synthetic polypeptide
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSLTEGRSEQYFGPGTRLTVT

SEQ ID NO: 678 - protein, 325 aa, synthetic construct
  REGION 1..325: note = Description of Artificial Sequence: Synthetic polypeptide
MEAVVTTLPREGGVRPSRKMLLLLLLLGPGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSAGGFSGNTIYFGEGSWLTVVEDLNKVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 679 - DNA, 346 bp, synthetic construct
  misc_feature 1..346: note = Description of Artificial Sequence: Synthetic polynucleotide
  source 1..346: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctgggggtttctctggaaacaccatatattttggagagggaagttggctcactgttgtag

SEQ ID NO: 680 - protein, 115 aa, synthetic construct
  REGION 1..115: note = Description of Artificial Sequence: Synthetic polypeptide
GAVVSQHPSRVICKSGTSVKIECRSLDFQATTMFWYRQFPKKSLMLMATSNEGSKATYEQGVEKDKFLINHASLTLSTLTVTSAHPEDSSFYICSAGGFSGNTIYFGEGSWLTVV

SEQ ID NO: 681 - protein, 9 aa, synthetic construct
  REGION 1..9: note = Description of Artificial Sequence: Synthetic peptide
VLNYGVCFC

SEQ ID NO: 682 - protein, 9 aa, synthetic construct
  REGION 1..9: note = Description of Artificial Sequence: Synthetic peptide
FCGDENILV

SEQ ID NO: 683 - protein, 95 aa, synthetic construct
  REGION 1..95: note = Description of Artificial Sequence: Synthetic polypeptide
MKDKQDEEQRTRRMMRTKMRMRRMRRTRRKMRRKMSPARPRTSCREACLQGWTEAAYEEAEDNCRRMMRTKAAYVLNYGVCFCAAYFCGDENILV

SEQ ID NO: 684 - protein, 9 aa, synthetic construct
  REGION 1..9: note = Description of Artificial Sequence: Synthetic peptide
RPRTSCREA

SEQ ID NO: 685 - protein, 9 aa, synthetic construct
  REGION 1..9: note = Description of Artificial Sequence: Synthetic peptide
SPARPRTSC

SEQ ID NO: 686 - protein, 9 aa, synthetic construct
  REGION 1..9: note = Description of Artificial Sequence: Synthetic peptide
RKMSPARPR

SEQ ID NO: 687 - protein, 9 aa, synthetic construct
  REGION 1..9: note = Description of Artificial Sequence: Synthetic peptide
MRRKMSPAR

SEQ ID NO: 688 - protein, 9 aa, synthetic construct
  REGION 1..9: note = Description of Artificial Sequence: Synthetic peptide
KMRRKMSPA

SEQ ID NO: 689 - protein, 9 aa, synthetic construct
  REGION 1..9: note = Description of Artificial Sequence: Synthetic peptide
TRRKMRRKM

SEQ ID NO: 690 - protein, 9 aa, synthetic construct
  REGION 1..9: note = Description of Artificial Sequence: Synthetic peptide
RTRRKMRRK

SEQ ID NO: 691 - protein, 9 aa, synthetic construct
  REGION 1..9: note = Description of Artificial Sequence: Synthetic peptide
RRTRRKMRR

SEQ ID NO: 692 - protein, 9 aa, synthetic construct
  REGION 1..9: note = Description of Artificial Sequence: Synthetic peptide
RMRRTRRKM

SEQ ID NO: 693 - protein, 9 aa, synthetic construct
  REGION 1..9: note = Description of Artificial Sequence: Synthetic peptide
RRMRRTRRK

SEQ ID NO: 694 - protein, 9 aa, synthetic construct
  REGION 1..9: note = Description of Artificial Sequence: Synthetic peptide
MRRMRRTRR

SEQ ID NO: 695 - protein, 9 aa, synthetic construct
  REGION 1..9: note = Description of Artificial Sequence: Synthetic peptide
MRMRRMRRT

SEQ ID NO: 696 - protein, 9 aa, synthetic construct
  REGION 1..9: note = Description of Artificial Sequence: Synthetic peptide
KMRMRRMRR

SEQ ID NO: 697 - protein, 9 aa, synthetic construct
  REGION 1..9: note = Description of Artificial Sequence: Synthetic peptide
MRTKMRMRR

SEQ ID NO: 698 - protein, 9 aa, synthetic construct
  REGION 1..9: note = Description of Artificial Sequence: Synthetic peptide
MMRTKMRMR

SEQ ID NO: 699 - protein, 9 aa, synthetic construct
  REGION 1..9: note = Description of Artificial Sequence: Synthetic peptide
RMMRTKMRM

SEQ ID NO: 700 - protein, 9 aa, synthetic construct
  REGION 1..9: note = Description of Artificial Sequence: Synthetic peptide
RRMMRTKMR

SEQ ID NO: 701 - protein, 9 aa, synthetic construct
  REGION 1..9: note = Description of Artificial Sequence: Synthetic peptide
RTRRMMRTK

SEQ ID NO: 702 - protein, 116 aa, synthetic construct
  REGION 1..116: note = Description of Artificial Sequence: Synthetic polypeptide
MACPGFLWALVISTCLEFSMAMKDKQDEEQRTRRMMRTKMRMRRMRRTRRKMRRKMSPARPRTSCREACLQGWTEAAYEEAEDNCRRMMRTKAAYVLNYGVCFCAAYFCGDENILV

SEQ ID NO: 703 - DNA, 20 bp, synthetic construct
  misc_feature 1..20: note = Description of Artificial Sequence: Synthetic oligonucleotide
  source 1..20: mol_type = other DNA
aatgatacggcgaccaccga

SEQ ID NO: 704 - DNA, 24 bp, synthetic construct
  misc_feature 1..24: note = Description of Artificial Sequence: Synthetic oligonucleotide
  source 1..24: mol_type = other DNA
caagcagaagacggcatacgagat

SEQ ID NO: 705 - skipped

SEQ ID NO: 706 - DNA, 34 bp, synthetic construct
  misc_feature 1..34: note = Description of Artificial Sequence: Synthetic oligonucleotide
  source 1..34: mol_type = other DNA
agatcggaagagcacacgtctgaactccagtcac